(19) United States
(12) Patent Application Publication
(10) Pub. No.: US 2021/0139906 A1
(43) Pub. Date: May 13, 2021

(54) COMPOUNDS AND METHODS FOR REDUCING SPDEF EXPRESSION

(71) Applicant: Ionis Pharmaceuticals, Inc., Carlsbad, CA (US)

(72) Inventors: Susan M. Freier, San Diego, CA (US); Huynh-Hoa Bui, San Diego, CA (US); Shuling Guo, Carlsbad, CA (US); Jeffrey R. Crosby, Carlsbad, CA (US)

(73) Assignee: Ionis Pharmaceuticals, Inc., Carlsbad, CA (US)

(21) Appl. No.: 17/147,215

(22) Filed: Jan. 12, 2021

Related U.S. Application Data
(63) Continuation of application No. PCT/US2020/059506, filed on Nov. 6, 2020.
(60) Provisional application No. 63/056,969, filed on Jul. 27, 2020.
(60) Provisional application No. 62/932,758, filed on Nov. 8, 2019.

(51) Int. Cl. C12N 15/113; C07H 21/04; A61K 9/00; A61K 47/02; A61P 11/00

(52) U.S. Cl. CPC C12N 15/113; C07H 21/04; A61K 9/0073; C12N 2310/351; A61P 11/00; C12N 2310/14; C12N 2310/3231; A61K 47/02

(57) ABSTRACT

Provided are compounds, methods, and pharmaceutical compositions for reducing the amount or activity of SPDEF RNA in a cell or subject, and in certain instances reducing the amount of SPDEF protein in a cell or subject. These compounds, methods, and pharmaceutical compositions are useful to ameliorate at least one symptom or hallmark of a disease or condition characterized by excessive mucus production or fibrosis, including cystic fibrosis, asthma, chronic obstructive pulmonary disease (COPD), pulmonary fibrosis, idiopathic pulmonary fibrosis (IPF), chronic bronchitis, rhinitis and ulcerative colitis.

### SEQUENCE LISTING

The present application is being filed along with a Sequence Listing in electronic format. The Sequence Listing is provided as a file entitled BIOL0366USC1SEQ_ST25.txt, created on Dec. 14, 2020, which is 569 kb in size. The information in the electronic format of the sequence listing is incorporated herein by reference in its entirety.

### FIELD

Provided are compounds, methods, and pharmaceutical compositions for ameliorating at least one symptom or hallmark of a disease or condition characterized by excessive mucus or fibrosis in a subject. In certain embodiments, there is excessive mucus in the nasal cavities (sinus), lung, gastrointestinal tract, or a combination thereof. Non-limiting examples of disease or conditions characterized by excessive mucus that may be treated with the compounds, methods, and pharmaceutical compositions disclosed herein are asthma, chronic obstructive pulmonary disease (COPD), chronic bronchitis, cystic fibrosis, and ulcerative colitis. Non-limiting examples of disease or conditions characterized by fibrosis that may be treated with the compounds, methods, and pharmaceutical compositions disclosed herein are pulmonary fibrosis and idiopathic pulmonary fibrosis (IPF).

### BACKGROUND

SAM Pointed Domain Containing ETS Transcription Factor (SPDEF) is a transcription factor that is critical for goblet cell differentiation in human lung tissue. SPDEF also regulates mucus production, inflammation, and airway responsiveness. SPDEF is expressed at low levels in the lung, but expression is increased when challenged with a virus or allergen. SPDEF expression is also increased in chronic lung disorders, such as cystic fibrosis, chronic bronchitis and asthma, relative to its expression in the lungs of subjects not diagnosed with such disorders. Chronic lung disorders are typically treated with bronchodilators, steroids and anti-inflammatory agents.

### SUMMARY OF THE INVENTION

Currently, there is a need for improved therapies and additional therapeutic options to treat disease or conditions characterized by excessive mucus or fibrosis. Often these disease or conditions result in dysfunction of the lungs and/or gastrointestinal tract. Non-limiting examples of such conditions include asthma, chronic obstructive pulmonary disease (COPD), cystic fibrosis, pulmonary fibrosis, idiopathic pulmonary fibrosis (IPF), and ulcerative colitis. It is therefore an object herein to provide compounds, methods, and pharmaceutical compositions for the treatment of such conditions.

Provided herein are compounds, methods and pharmaceutical compositions for reducing the amount or activity of SPDEF RNA in a cell or a subject. In general, compounds and pharmaceutical compositions comprise an oligomeric compound capable of reducing expression of SPDEF RNA. In certain embodiments, compounds, methods and pharmaceutical compositions reduce the amount or activity of SPDEF protein in a cell or a subject.

Provided herein are compounds, methods and pharmaceutical compositions for ameliorating at least one symptom or hallmark of a disease or condition characterized by excessive mucus or fibrosis in a subject. In certain embodiments, the disease or condition is cystic fibrosis. In certain embodiments, the disease or condition is a gastrointestinal condition, e.g., ulcerative colitis. In certain embodiments, the disease or condition is a pulmonary condition. Non-limiting examples of such pulmonary conditions are bronchitis, asthma, COPD, pulmonary fibrosis, idiopathic pulmonary fibrosis (IPF), pneumonia, emphysema, rhinitis, sinusitis, nasal polyposis, sinus polyposis, bronchiectasis, and sarcoidosis.

### DETAILED DESCRIPTION OF THE INVENTION

It is to be understood that both the foregoing general description and the following detailed description are exemplary and explanatory only and are not restrictive. Herein, the use of the singular includes the plural unless specifically stated otherwise. As used herein, the use of “or” means “and/or” unless stated otherwise. Furthermore, the use of the term “including” as well as other forms, such as “includes” and “included”, is not limiting. Also, terms such as “element” or “component” encompass both elements and components comprising one unit and elements and components that comprise more than one subunit, unless specifically stated otherwise.

The section headings used herein are for organizational purposes only and are not to be construed as limiting the subject matter described. All documents, or portions of documents, cited in this application, including, but not limited to, patents, patent applications, articles, books, and treatises, are hereby expressly incorporated-by-reference for the portions of the document discussed herein, as well as in their entirety.

### Definitions

Unless specific definitions are provided, the nomenclature used in connection with, and the procedures and techniques of, analytical chemistry, synthetic organic chemistry, and medicinal and pharmaceutical chemistry described herein are those well-known and commonly used in the art. Where permitted, all patents, applications, published applications and other publications and other data referred to throughout in the disclosure are incorporated by reference herein in their entirety.

Unless otherwise indicated, the following terms have the following meanings:

### Definitions

As used herein, “2′-deoxynucleoside” means a nucleoside comprising a 2′-H(H) deoxyribosyl sugar moiety. In certain embodiments, a 2′-deoxynucleoside is a 2′-β-D-deoxynucleoside and comprises a 2′-β-D-deoxyribosyl sugar moiety, which has the β-D configuration as found in naturally occurring deoxyribonucleic acids (DNA). In certain embodiments, a 2′-deoxynucleoside or nucleoside comprising an unmodified 2′-deoxyribosyl sugar moiety may comprise a modified nucleobase or may comprise an RNA nucleobase (uracil).

As used herein, “2′-MOE” or “2′-MOE sugar moiety” means a 2′-OCH₂CH₂OCH₃ group in place of the 2′-OH group of a ribosyl sugar moiety. “MOE” means methoxyethyl.

As used herein, “2′-MOE nucleoside” means a nucleoside comprising a 2′-MOE sugar moiety.

As used herein, “2′-OMe” or “2′-O-methyl sugar moiety” means a 2′-OCH₃ group in place of the 2′-OH group of a ribosyl sugar moiety.

As used herein, “2′-OMe nucleoside” means a nucleoside comprising a 2′-OMe sugar moiety.

As used herein, “2′-substituted nucleoside” means a nucleoside comprising a 2′-substituted sugar moiety. As used herein, “2′-substituted” in reference to a sugar moiety means a sugar moiety comprising at least one 2′-substituent group other than H or OH.

As used herein, “5-methyl cytosine” means a cytosine modified with a methyl group attached to the 5 position. A 5-methyl cytosine is a modified nucleobase.

As used herein, “administering” means providing a pharmaceutical agent to a subject.

As used herein, “antisense activity” means any detectable and/or measurable change attributable to the hybridization of an antisense compound to its target nucleic acid. In certain embodiments, antisense activity is a decrease in the amount or expression of a target nucleic acid or protein encoded by such target nucleic acid compared to target nucleic acid levels or target protein levels in the absence of the antisense compound.

As used herein, “antisense compound” means an oligomeric compound capable of achieving at least one antisense activity.

As used herein, “ameliorate” in reference to a treatment means improvement in at least one symptom relative to the same symptom in the absence of the treatment. In certain embodiments, amelioration is the reduction in the severity or frequency of a symptom or the delayed onset or slowing of progression in the severity or frequency of a symptom.

As used herein, “bicyclic nucleoside” or “BNA” means a nucleoside comprising a bicyclic sugar moiety.

As used herein, “bicyclic sugar” or “bicyclic sugar moiety” means a modified sugar moiety comprising two rings, wherein the second ring is formed via a bridge connecting two of the atoms in the first ring thereby forming a bicyclic structure. In certain embodiments, the first ring of the bicyclic sugar moiety is a furanosyl moiety. In certain embodiments, the bicyclic sugar moiety does not comprise a furanosyl moiety.

As used herein, “cleavable moiety” means a bond or group of atoms that is cleaved under physiological conditions, for example, inside a cell or a subject.

As used herein, “complementary” in reference to an oligonucleotide means that at least 70% of the nucleobases of the oligonucleotide or one or more regions thereof and the nucleobases of another nucleic acid or one or more regions thereof are capable of hydrogen bonding with one another when the nucleobase sequence of the oligonucleotide and the other nucleic acid are aligned in opposing directions. As used herein, “complementary nucleobases” means nucleobases that are capable of forming hydrogen bonds with one another. Complementary nucleobase pairs include adenine (A) with thymine (T), adenine (A) with uracil (U), cytosine (C) with guanine (G), and 5-methyl cytosine (mC) with guanine (G). Complementary oligonucleotides and/or nucleic acids need not have nucleobase complementarity at each nucleoside. Rather, some mismatches are tolerated. As used herein, “fully complementary” or “100% complementary” in reference to an oligonucleotide, or portion thereof, means that oligonucleotide, or portion thereof, is complementary to another oligonucleotide or nucleic acid at each nucleobase of the oligonucleotide.

As used herein, “conjugate group” means a group of atoms that is directly or indirectly attached to an oligonucleotide. Conjugate groups include a conjugate moiety and a conjugate linker that attaches the conjugate moiety to the oligonucleotide.

As used herein, “conjugate linker” means a single bond or a group of atoms comprising at least one bond that connects a conjugate moiety to an oligonucleotide.

As used herein, “conjugate moiety” means a group of atoms that is attached to an oligonucleotide via a conjugate linker.

As used herein, “contiguous” in the context of an oligonucleotide refers to nucleosides, nucleobases, sugar moieties, or internucleoside linkages that are immediately adjacent to each other. For example, “contiguous nucleobases” means nucleobases that are immediately adjacent to each other in a sequence.

As used herein, “constrained ethyl” or “cEt” or “cEt modified sugar” means a β-D ribosyl bicyclic sugar moiety wherein the second ring of the bicyclic sugar is formed via a bridge connecting the 4′-carbon and the 2′-carbon of the β-D ribosyl sugar moiety, wherein the bridge has the formula 4′-CH(CH₃)—O-2′, and wherein the methyl group of the bridge is in the S configuration.

As used herein, “cEt nucleoside” means a nucleoside comprising cEt modified sugar moiety.

As used herein, “chirally enriched population” means a plurality of molecules of identical molecular formula, wherein the number or percentage of molecules within the population that contain a particular stereochemical configuration at a particular chiral center is greater than the number or percentage of molecules expected to contain the same particular stereochemical configuration at the same particular chiral center within the population if the particular chiral center were stereorandom. Chirally enriched populations of molecules having multiple chiral centers within each molecule may contain one or more stereorandom chiral centers. In certain embodiments, the molecules are modified oligonucleotides. In certain embodiments, the molecules are compounds comprising modified oligonucleotides.

As used herein, “double-stranded” refers to a region of hybridized nucleic acid(s). In certain embodiments, such double-strand results from hybridization of an oligonucleotide (or portion thereof) to a target region of a transcript. In certain embodiments, a double-strand results from hybridization of two oligonucleotides (or portions thereof) to one another. In certain embodiments, the hybridized regions are portions (including the entirety) of two separate molecules (e.g., no covalent bond connects the two complementary strands together). In certain embodiments, the hybridized regions are portions of the same molecule that have hybridized (e.g., a hairpin structure).

As used herein, “duplex” means a structure formed by two separate nucleic acid molecules at least a portion of which are complementary and that are hybridized to one another, but are not covalently bonded to one another.

As used herein, “gapmer” means a modified oligonucleotide comprising an internal region having a plurality of nucleosides that support RNase H cleavage positioned between external regions having one or more nucleosides, wherein the nucleosides comprising the internal region are chemically distinct from the nucleoside or nucleosides comprising the external regions. The internal region may be referred to as the “gap” and the external regions may be referred to as the “wings.” Unless otherwise indicated, “gapmer” refers to a sugar motif Unless otherwise indicated, the sugar moiety of each nucleoside of the gap is a 2′-β-D-deoxyribosyl sugar moiety. Thus, the term “cEt gapmer” indicates a gapmer having a gap comprising 2′-β-D-deoxynucleosides and wings comprising a cEt nucleoside. Unless otherwise indicated, a cEt gapmer may comprise one or more modified internucleoside linkages and/or modified nucleobases and such modifications do not necessarily follow the gapmer pattern of the sugar modifications.

As used herein, “hotspot region” is a range of nucleobases on a target nucleic acid that is amenable to oligomeric compound-mediated reduction of the amount or activity of the target nucleic acid.

As used herein, “hybridization” means the pairing or annealing of complementary oligonucleotides and/or nucleic acids. While not limited to a particular mechanism, the most common mechanism of hybridization involves hydrogen bonding, which may be Watson-Crick, Hoogsteen or reversed Hoogsteen hydrogen bonding, between complementary nucleobases.

As used herein, “internucleoside linkage” means the covalent linkage between contiguous nucleosides in an oligonucleotide. As used herein “modified internucleoside linkage” means any internucleoside linkage other than a phosphodiester internucleoside linkage. “Phosphorothioate internucleoside linkage” is a modified internucleoside linkage in which one of the non-bridging oxygen atoms of a phosphodiester internucleoside linkage is replaced with a sulfur atom.

As used herein, “inverted nucleoside” means a nucleotide having a 3′ to 3′ and/or 5′ to 5′ internucleoside linkage, as shown herein.

As used herein, “inverted sugar moiety” means the sugar moiety of an inverted nucleoside or an abasic sugar moiety having a 3′ to 3′ and/or 5′ to 5′ internucleoside linkage.

As used herein, “linker-nucleoside” means a nucleoside that links, either directly or indirectly, an oligonucleotide to a conjugate moiety. Linker-nucleosides are located within the conjugate linker of an oligomeric compound. Linker-nucleosides are not considered part of the oligonucleotide portion of an oligomeric compound even if they are contiguous with the oligonucleotide.

“Lipid nanoparticle” or “LNP” is a vesicle comprising a lipid layer encapsulating a pharmaceutically active molecule, such as a nucleic acid molecule, e.g., an RNAi or a plasmid from which an RNAi is transcribed. LNPs are described in, for example, U.S. Pat. Nos. 6,858,225, 6,815,432, 8,158,601, and 8,058,069, the entire contents of which are hereby incorporated herein by reference.

As used herein, “non-bicyclic modified sugar moiety” means a modified sugar moiety that comprises a modification, such as a substituent, that does not form a bridge between two atoms of the sugar to form a second ring.

As used herein, “mismatch” or “non-complementary” means a nucleobase of a first oligonucleotide that is not complementary with the corresponding nucleobase of a second oligonucleotide or target nucleic acid when the first and second oligonucleotide are aligned.

As used herein, “motif” means the pattern of unmodified and/or modified sugar moieties, nucleobases, and/or internucleoside linkages, in an oligonucleotide.

As used herein, “nucleobase” means an unmodified nucleobase or a modified nucleobase. As used herein an “unmodified nucleobase” is adenine (A), thymine (T), cytosine (C), uracil (U), or guanine (G). As used herein, a “modified nucleobase” is a group of atoms other than unmodified A, T, C, U, or G capable of pairing with at least one unmodified nucleobase. A “5-methyl cytosine” is a modified nucleobase. A universal base is a modified nucleobase that can pair with any one of the five unmodified nucleobases. As used herein, “nucleobase sequence” means the order of contiguous nucleobases in a nucleic acid or oligonucleotide independent of any sugar or internucleoside linkage modification.

As used herein, “nucleoside” means a compound comprising a nucleobase and a sugar moiety. The nucleobase and sugar moiety are each, independently, unmodified or modified. As used herein, “modified nucleoside” means a nucleoside comprising a modified nucleobase and/or a modified sugar moiety. Modified nucleosides include abasic nucleosides, which lack a nucleobase. “Linked nucleosides” are nucleosides that are connected in a contiguous sequence (i.e., no additional nucleosides are presented between those that are linked).

As used herein, “overhang” refers to unpaired nucleotides at either or both ends of a duplex formed by hybridization of an antisense RNAi oligonucleotide and a sense RNAi oligonucleotide.

As used herein, “oligomeric compound” means an oligonucleotide and optionally one or more additional features, such as a conjugate group or terminal group. An oligomeric compound may be paired with a second oligomeric compound that is complementary to the first oligomeric compound or may be unpaired. A “singled-stranded oligomeric compound” is an unpaired oligomeric compound. The term “oligomeric duplex” means a duplex formed by two oligomeric compounds having complementary nucleobase sequences. Each oligomeric compound of an oligomeric duplex may be referred to as a “duplexed oligomeric compound.”

As used herein, “oligonucleotide” means a strand of linked nucleosides connected via internucleoside linkages, wherein each nucleoside and internucleoside linkage may be modified or unmodified. Unless otherwise indicated, oligonucleotides consist of 8-50 linked nucleosides. As used herein, “modified oligonucleotide” means an oligonucleotide, wherein at least one nucleoside or internucleoside linkage is modified. As used herein, “unmodified oligonucleotide” means an oligonucleotide that does not comprise any nucleoside modifications or internucleoside modifications.

As used herein, “pharmaceutically acceptable carrier or diluent” means any substance suitable for use in administering to a subject. Certain such carriers enable pharmaceutical compositions to be formulated as, for example, tablets, pills, dragees, capsules, liquids, gels, syrups, slurries, suspension and lozenges for the oral ingestion by a subject. In certain embodiments, a pharmaceutically acceptable carrier or diluent is sterile water, sterile saline, sterile buffer solution or sterile artificial cerebrospinal fluid.

As used herein “pharmaceutically acceptable salts” means physiologically and pharmaceutically acceptable salts of compounds. Pharmaceutically acceptable salts retain the desired biological activity of the parent compound and do not impart undesired toxicological effects thereto.

As used herein “pharmaceutical composition” means a mixture of substances suitable for administering to a subject. For example, a pharmaceutical composition may comprise an oligomeric compound and a sterile aqueous solution. In certain embodiments, a pharmaceutical composition shows activity in a free uptake assay in certain cell lines.

As used herein “prodrug” means a therapeutic agent in a form outside the body that is converted to a different form within a subject or cells thereof. Typically, conversion of a prodrug within the subject is facilitated by the action of an enzymes (e.g., endogenous or viral enzyme) or chemicals present in cells or tissues and/or by physiologic conditions.

As used herein, “reducing or inhibiting the amount or activity” refers to a reduction or blockade of the transcriptional expression or activity relative to the transcriptional expression or activity in an untreated or control sample and does not necessarily indicate a total elimination of transcriptional expression or activity.

As used herein, “RNA” means an RNA transcript and includes pre-mRNA and mature mRNA unless otherwise specified.

As used herein, “RNAi compound” means an antisense compound that acts, at least in part, through RISC or Ago2 to modulate a target nucleic acid and/or protein encoded by a target nucleic acid. RNAi compounds include, but are not limited to double-stranded siRNA, single-stranded RNA (ssRNA), and microRNA, including microRNA mimics. In certain embodiments, an RNAi compound modulates the amount, activity, and/or splicing of a target nucleic acid. The term RNAi compound excludes antisense compounds that act through RNase H.

As used herein, “RNAi oligonucleotide” means an antisense RNAi oligonucleotide or a sense RNAi oligonucleotide.

As used herein, “antisense RNAi oligonucleotide” means an oligonucleotide comprising a region that is complementary to a target sequence, and which includes at least one chemical modification suitable for RNAi.

As used herein, “sense RNAi oligonucleotide” means an oligonucleotide comprising a region that is complementary to a region of an antisense RNAi oligonucleotide, and which is capable of forming a duplex with such antisense RNAi oligonucleotide. A duplex formed by an antisense RNAi oligonucleotide and a sense RNAi oligonucleotide is referred to as a double-stranded RNAi compound (dsRNAi) or a short interfering RNA (siRNA).

As used herein, “antisense RNase H oligonucleotide” means an oligonucleotide comprising a region that is complementary to a target sequence, and which includes at least one chemical modification suitable for RNase H-mediated nucleic acid reduction.

As used herein, “self-complementary” in reference to an oligonucleotide means an oligonucleotide that at least partially hybridizes to itself.

As used herein, “stabilized phosphate group” means a 5′-phosphate analog that is metabolically more stable than a 5′-phosphate as naturally occurs on DNA or RNA.

As used herein, “standard cell assay” means the assay described in Example 1 and reasonable variations thereof.

As used herein, “stereorandom” in the context of a population of molecules of identical molecular formula means a chiral center having a random stereochemical configuration. For example, in a population of molecules comprising a stereorandom chiral center, the number of molecules having the (S) configuration of the stereorandom chiral center may be but is not necessarily the same as the number of molecules having the (R) configuration of the stereorandom chiral center. The stereochemical configuration of a chiral center is considered random when it is the result of a synthetic method that is not designed to control the stereochemical configuration. In certain embodiments, a stereorandom chiral center is a stereorandom phosphorothioate internucleoside linkage.

As used herein, “subject” means a human or non-human animal. In certain embodiments, the subject is a human.

As used herein, “sugar moiety” means an unmodified sugar moiety or a modified sugar moiety. As used herein, “unmodified sugar moiety” means a 2′-OH(H) ribosyl moiety, as found in RNA (an “unmodified RNA sugar moiety”), or a 2′-H(H) deoxyribosyl moiety, as found in DNA (an “unmodified DNA sugar moiety”). Unmodified sugar moieties have one hydrogen at each of the 1′, 3′, and 4′ positions, an oxygen at the 3′ position, and two hydrogens at the 5′ position. As used herein, “modified sugar moiety” or “modified sugar” means a modified furanosyl sugar moiety or a sugar surrogate.

As used herein, “sugar surrogate” means a modified sugar moiety having other than a furanosyl moiety that can link a nucleobase to another group, such as an internucleoside linkage, conjugate group, or terminal group in an oligonucleotide. Modified nucleosides comprising sugar surrogates can be incorporated into one or more positions within an oligonucleotide and such oligonucleotides are capable of hybridizing to complementary oligomeric compounds or nucleic acids.

As used herein, “symptom or hallmark” means any physical feature or test result that indicates the existence or extent of a disease or disorder. In certain embodiments, a symptom is apparent to a subject or to a medical professional examining or testing said subject. In certain embodiments, a hallmark is apparent upon invasive diagnostic testing, including, but not limited to, post-mortem tests.

As used herein, “target nucleic acid” and “target RNA” mean a nucleic acid that an antisense compound is designed to affect. In certain embodiments, the target RNA is a SPDEF RNA, and the nucleic acid is a SPDEF nucleic acid.

As used herein, “target region” means a portion of a target nucleic acid to which an oligomeric compound is designed to hybridize.

As used herein, “terminal group” means a chemical group or group of atoms that is covalently linked to a terminus of an oligonucleotide.

As used herein, “therapeutically effective amount” means an amount of a pharmaceutical agent that provides a therapeutic benefit to a subject. For example, a therapeutically effective amount improves a symptom or hallmark of a disease.

### Certain Embodiments

The present disclosure provides the following non-limiting numbered embodiments:

Embodiment 1. An oligomeric compound comprising a modified oligonucleotide consisting of 12 to 50, 12 to 45, 12 to 40, 12 to 35, 12 to 30, 12 to 25, or 12 to 20 linked nucleosides, wherein the nucleobase sequence of the modified oligonucleotide is at least 90% complementary to an equal length portion of an SPDEF nucleic acid, and wherein the modified oligonucleotide comprises at least one modification selected from a modified sugar moiety and a modified internucleoside linkage.
Embodiment 2. An oligomeric compound comprising a modified oligonucleotide consisting of 12 to 50, 12 to 45, 12 to 40, 12 to 35, 12 to 30, 12 to 25, or 12 to 20 linked nucleosides and having a nucleobase sequence comprising at least 8, 9, 10, 11, 12, 13, 14, 15, 16, 17, 18, 19, or 20 contiguous nucleobases complementary to an equal length portion of the nucleobase sequence of any of SEQ ID NOS: 1-5.
Embodiment 3. An oligomeric compound comprising a modified oligonucleotide consisting of 12 to 50, 12 to 45, 12 to 40, 12 to 35, 12 to 30, 12 to 25, or 12 to 20 linked nucleosides and having a nucleobase sequence comprising at least 8, at least 9, at least 10, at least 11, at least 12, at least 13, at least 14, at least 15, at least 16, at least 17, at least 18, at least 19, or at least 20 contiguous nucleobases complementary to: an equal length portion of nucleobases 3521-3554 of SEQ ID NO: 2; an equal length portion of nucleobases 3684-3702 of SEQ ID NO: 2; an equal length portion of nucleobases 3785-3821 of SEQ ID NO: 2; an equal length portion of nucleobases 6356-6377 of SEQ ID NO: 2; an equal length portion of nucleobases 8809-8826 of SEQ ID NO: 2; an equal length portion of nucleobases 9800-9817 of SEQ ID NO: 2; an equal length portion of nucleobases 14212-14231 of SEQ ID NO: 2; an equal length portion of nucleobases 15385-15408 of SEQ ID NO: 2; an equal length portion of nucleobases 17289-17307 of SEQ ID NO: 2; or an equal length portion of nucleobases 17490-17509 of SEQ ID NO: 2.
Embodiment 4. The oligomeric compound of embodiment 3, wherein the modified oligonucleotide comprises at least 8, at least 9, at least 10, at least 11, at least 12, at least 13, at least 14, at least 15, at least 16, at least 17, at least 18, at least 19, or at least 20 contiguous nucleobases of a sequence selected from: SEQ ID NOS: 1053, 1129, 2166, 2167, 2168, 2169, 2170, 2171, 2172, 2173, 2174, 2175, 2176, 2242, and 2247; SEQ ID NOS: 1777, 1852, 1928, and 2004; SEQ ID NOS: 1282, 1358, 1434, 2177, 2178, 2179, 2180, 2181, 2182, 2183, 2184, 2185, and 2186; SEQ ID NOS: 678, 2198, 2199, 2200, 2244, and 2248; SEQ ID NOS: 683, 1715, and 2245; SEQ ID NOS: 761, 2229, and 2230; SEQ ID NOS: 1606, 1682, 2255, 2275, and 2280; SEQ ID NOS: 999, 1075, 2262, 2263, 2264, 2265, 2266, 2267, and 2268; SEQ ID NOS: 163, 1980, 2056, and 2277; or SEQ ID NOS: 1831, 1907, 1983, 2059, and 2282.
Embodiment 5. The oligomeric compound of any one of embodiments 1-4, wherein the modified oligonucleotide has a nucleobase sequence that is at least 80%, 85%, 90%, 95%, or 100% complementary to an equal length portion of a nucleobase sequence selected from SEQ ID NOS: 1-5 when measured across the entire nucleobase sequence of the modified oligonucleotide.
Embodiment 6. The oligomeric compound of any one of embodiments 1-5, wherein at least one modified nucleoside comprises a modified sugar moiety.
Embodiment 7. The oligomeric compound of embodiment 6, wherein the modified sugar moiety comprises a bicyclic sugar moiety.
Embodiment 8. The oligomeric compound of embodiment 7, wherein the bicyclic sugar moiety comprises a 2′-4′ bridge selected from —O—CH2-; and —O—CH(CH3)-.
Embodiment 9. The oligomeric compound of embodiment 6, wherein the modified sugar moiety comprises a non-bicyclic modified sugar moiety.
Embodiment 10. The oligomeric compound of embodiment 9, wherein the non-bicyclic modified sugar moiety comprises a 2′-MOE sugar moiety or 2′-OMe sugar moiety.
Embodiment 11. The oligomeric compound of any one of embodiments 1-5, wherein at least one modified nucleoside comprises a sugar surrogate.
Embodiment 12. The oligomeric compound of embodiment 11, wherein the sugar surrogate is selected from morpholino and PNA.
Embodiment 13. The oligomeric compound of any of embodiments 1-12, wherein the modified oligonucleotide has a sugar motif comprising: a 5′-region consisting of 1-5 linked 5′-region nucleosides; a central region consisting of 6-10 linked central region nucleosides; and a 3′-region consisting of 1-5 linked 3′-region nucleosides; wherein each of the 5′-region nucleosides and each of the 3′-region nucleosides comprises a modified sugar moiety and each of the central region nucleosides comprises an unmodified 2′-deoxyribosyl sugar moiety.
Embodiment 14. The oligomeric compound of any one of embodiments 1-13, wherein the modified oligonucleotide comprises at least one modified internucleoside linkage.
Embodiment 15. The oligomeric compound of embodiment 14, wherein the modified internucleoside linkage is a phosphorothioate internucleoside linkage.
Embodiment 16. The oligomeric compound of embodiment 14, wherein each internucleoside linkage of the modified oligonucleotide is a modified internucleoside linkage.
Embodiment 17. The oligomeric compound of any one of embodiments 1-13, wherein each internucleoside linkage of the modified oligonucleotide is a phosphorothioate internucleoside linkage.
Embodiment 18. The oligomeric compound of any one of embodiments 1-13, wherein the modified oligonucleotide comprises at least one phosphodiester internucleoside linkage.
Embodiment 19. The oligomeric compound of embodiment 14, wherein each internucleoside linkage is independently selected from a phosphodiester internucleoside linkage or a phosphorothioate internucleoside linkage.
Embodiment 20. The oligomeric compound of any of embodiments 1-19, wherein the modified oligonucleotide comprises at least one modified nucleobase.
Embodiment 21. The oligomeric compound of embodiment 20, wherein the modified nucleobase is a 5-methyl cytosine.
Embodiment 22. The oligomeric compound of any of embodiments 1-21, wherein the modified oligonucleotide consists of 12-30, 12-22, 12-20, 14-20, 15-25, 16-20, 18-22 or 18-20 linked nucleosides.
Embodiment 23. The oligomeric compound of any of embodiments 1-22, wherein the modified oligonucleotide consists of 16 linked nucleosides.
Embodiment 24. The oligomeric compound of embodiment 23, wherein each of nucleosides 1-3 and 14-16 (from 5′ to 3′) comprise a cEt modification and each of nucleosides 4-13 are 2′-deoxynucleosides.
Embodiment 25. The oligomeric compound of embodiment 23, wherein each of nucleosides 1-2 and 15-16 (from 5′ to 3′) comprise a cEt modification and each of nucleosides 3-14 are 2′-deoxynucleosides.
Embodiment 26. The oligomeric compound of any of embodiments 1-25, consisting of the modified oligonucleotide.
Embodiment 27. The oligomeric compound of any of embodiments 1-25, comprising a conjugate group comprising a conjugate moiety and a conjugate linker.
Embodiment 28. The oligomeric compound of embodiment 27, wherein the conjugate group comprises a GalNAc cluster comprising 1-3 GalNAc ligands.
Embodiment 29. The oligomeric compound of embodiments 27 or 28, wherein the conjugate linker consists of a single bond.
Embodiment 30. The oligomeric compound of embodiment 27, wherein the conjugate linker is cleavable.
Embodiment 31. The oligomeric compound of embodiment 30, wherein the conjugate linker comprises 1-3 linker-nucleosides.
Embodiment 32. The oligomeric compound of any of embodiments 27-31, wherein the conjugate group is attached to the modified oligonucleotide at the 5′-end of the modified oligonucleotide.
Embodiment 33. The oligomeric compound of any of embodiments 27-31, wherein the conjugate group is attached to the modified oligonucleotide at the 3′-end of the modified oligonucleotide.
Embodiment 34. The oligomeric compound of any of embodiments 1-33 comprising a terminal group.
Embodiment 35. The oligomeric compound of any of embodiments 1-34 wherein the oligomeric compound is a single-stranded oligomeric compound.
Embodiment 36. The oligomeric compound of any of embodiments 1-30 or 32-35, wherein the oligomeric compound does not comprise linker-nucleosides.
Embodiment 37. An oligomeric duplex comprising an oligomeric compound of any of embodiments 1-34 or 36.
Embodiment 38. An antisense compound comprising or consisting of an oligomeric compound of any of embodiments 1-36 or an oligomeric duplex of embodiment 37.
Embodiment 39. A modified oligonucleotide according to the following chemical structure:

[chemical structure]

Embodiment 40. A modified oligonucleotide according to the following chemical structure:

[chemical structure]

Embodiment 41. A modified oligonucleotide according to the following chemical structure:

[chemical structure]

or a salt thereof.

Embodiment 42. A modified oligonucleotide according to the following chemical structure:

[chemical structure]

or a salt thereof.

Embodiment 43. The modified oligonucleotide of embodiment 41 or 42, which is a sodium salt.
Embodiment 44. A modified oligonucleotide according to the following chemical structure:

[chemical structure]

Embodiment 45. A modified oligonucleotide according to the following chemical notation:
mCks Aks Aks Tds Ads Ads Gds mCds Ads Ads Gds Tds mCds Tks Gks Gks; wherein
   A=an adenine nucleobase
   mC=a 5′-methyl cytosine nucleobase
   G=a guanine nucleobase
   T=a thymine nucleobase
   k=a cEt modified sugar
   d=a 2′-deoxyribose sugar, and
   s=a phosphorothioate internucleoside linkage.
      Embodiment 46. A modified oligonucleotide according to the following chemical notation:
Aks mCks Tds Tds Gds Tds Ads Ads mCds Ads Gds Tes Ges Ges Tks Tk; wherein
   A=an adenine nucleobase
   mC=a 5′-methyl cytosine nucleobase
   G=a guanine nucleobase
   T=a thymine nucleobase
   k=a cEt modified sugar
   d=a 2′-deoxyribose sugar, and
   s=a phosphorothioate internucleoside linkage.
      Embodiment 47. A pharmaceutical composition comprising the oligomeric compound of any of embodiments 1-36, the oligomeric duplex of embodiment 37, the antisense compound of embodiment 38, or the modified oligonucleotides of any one of embodiments 39-46; and a pharmaceutically acceptable carrier or diluent.
      Embodiment 48. The pharmaceutical composition of embodiment 47, wherein the pharmaceutically acceptable carrier or diluent comprises phosphate buffered saline.
      Embodiment 49. The pharmaceutical composition of embodiment 48, consisting essentially of the oligomeric compound, antisense compound or oligomeric duplex, and phosphate buffered saline.
      Embodiment 50. A method comprising administering to a subject the oligomeric compound of any of embodiments 1-36, the oligomeric duplex of embodiment 37, the antisense compound of embodiment 38, the modified oligonucleotides of any one of embodiments 39-46, or the pharmaceutical composition of any of embodiments 47-49.
      Embodiment 51. A method of treating a pulmonary condition comprising administering to a subject having or at risk for developing the pulmonary condition a therapeutically effective amount of the oligomeric compound of any of embodiments 1-36, the oligomeric duplex of embodiment 37, the antisense compound of embodiment 38, the modified oligonucleotides of any one of embodiments 39-46, or the pharmaceutical composition according to any of embodiments 47-49, thereby treating the pulmonary condition.
      Embodiment 52. A method of reducing SPDEF RNA or SPDEF protein in a lung of a subject having or at risk for developing a pulmonary condition comprising administering a therapeutically effective amount of the oligomeric compound of any of embodiments 1-36, the oligomeric duplex of embodiment 37, the antisense compound of embodiment 38, the modified oligonucleotides of any one of embodiments 39-46, or the pharmaceutical composition according to any of embodiments 47-49, thereby reducing SPDEF RNA or SPDEF protein in the lung.
      Embodiment 53. The method of embodiment 51 or 52, wherein the pulmonary condition is selected from bronchitis, asthma, chronic obstructive pulmonary disease, pulmonary fibrosis, idiopathic pulmonary fibrosis (IPF), pneumonia, emphysema, rhinitis, sinusitis, nasal polyposis, sinus polyposis, bronchiectasis, and sarcoidosis.
      Embodiment 54. The method of embodiment 51 or 52, wherein the pulmonary condition is chronic bronchitis.
      Embodiment 55. The method of embodiment 51 or 52, wherein the pulmonary condition is severe asthma.
      Embodiment 56. The method of any one of embodiments 51-55, wherein the administering comprises administering via nebulizer or inhaler.
      Embodiment 57. The method of any one of embodiments 51-56, wherein at least one symptom or hallmark of the pulmonary condition is ameliorated.
      Embodiment 58. The method of embodiment 57, wherein the symptom or hallmark is selected from shortness of breath, chest pain, coughing, wheezing, fatigue, and sleep disruption.
      Embodiment 59. The method of any of embodiments 51-58, wherein the method prevents or slows disease progression.
      Embodiment 60. A method of reducing mucus production in the lungs of a subject, the method comprising administering the oligomeric compound of any of embodiments 1-36, the oligomeric duplex of embodiment 37, the antisense compound of embodiment 38, the modified oligonucleotides of any one of embodiments 39-46; or the pharmaceutical composition of any one of embodiments 47-49.
      Embodiment 61. The method of any one of embodiments 50-60, wherein administering comprises oral delivery or nasal delivery.
      Embodiment 62. The method of any one of embodiments 50-61, wherein administering comprises aerosolized delivery.
      Embodiment 63. Use of the oligomeric compound of any of embodiments 1-36, the oligomeric duplex of embodiment 37, the antisense compound of embodiment 38, the modified oligonucleotides of any one of embodiments 39-46; or the pharmaceutical composition of any one of embodiments 47-49 for the treatment of a pulmonary condition.
      Embodiment 64. The use of embodiment 60, wherein the pulmonary condition is selected from bronchitis, asthma, chronic obstructive pulmonary disease, pneumonia, emphysema, rhinitis, sinusitis, nasal polyposis, sinus polyposis, bronchiectasis, and sarcoidosis.
      Embodiment 65. The use of embodiment 63, wherein the pulmonary condition is chronic bronchitis.
      Embodiment 66. The use of embodiment 63, wherein the pulmonary condition is severe asthma.
      Embodiment 67. A method of reducing mucus production in the gastrointestinal tract of a subject, the method comprising administering the oligomeric compound of any of embodiments 1-36, the oligomeric duplex of embodiment 37, the antisense compound of embodiment 38, the modified oligonucleotides of any one of embodiments 39-46; or the pharmaceutical composition of any one of embodiments 47-49.
      Embodiment 68. A method of treating a gastrointestinal condition comprising administering to a subject having or at risk for developing the gastrointestinal condition a therapeutically effective amount of the pharmaceutical composition according to any of embodiments 47-49, thereby treating the gastrointestinal condition.
      Embodiment 69. A method of reducing SPDEF RNA or SPDEF protein in the gastrointestinal tract of a subject having or at risk for developing a gastrointestinal condition, the method comprising administering a therapeutically effective amount of the pharmaceutical composition according to any of embodiments 47-49, thereby reducing SPDEF RNA or SPDEF protein in the gastrointestinal tract.
      Embodiment 70. The method of embodiment 68 or 69, wherein the gastrointestinal condition is ulcerative colitis.
      Embodiment 71. A method of reducing inflammation in a subject in need thereof, wherein the method comprises administering a therapeutically effective amount of the oligomeric compound of any of claims 1-36, the oligomeric duplex of claim 37, the antisense compound of claim 38, or the modified oligonucleotides of any one of claims 39-46, or the pharmaceutical composition of any one of claim 47-49.
      Embodiment 72. The method of claim 71, wherein administering reduces inflammation in a lung of the subject.
      Embodiment 73. The method of claim 71, wherein administering reduces inflammation in the gastrointestinal tract of the subject.
      Embodiment 74. A system for treating a pulmonary condition comprising: a nebulizer or an inhaler; the oligomeric compound of any one of embodiments 1-36, the oligomeric duplex of embodiment 37, the antisense compound of embodiment 38, or the modified oligonucleotide of any one of embodiments 39-46; and a pharmaceutically acceptable carrier or diluent.
      Embodiment 75. An oligomeric compound comprising a modified oligonucleotide consisting of 12 to 30 linked nucleosides, wherein the nucleobase sequence of the modified oligonucleotide comprises at least 12, 13, 14, 15, 16, 17, 18, 19, 20, 21, 22 or 23 nucleobases of any of SEQ ID NOS: 2324-2510; wherein the modified oligonucleotide comprises at least one modification selected from a modified sugar and a modified internucleoside linkage.
      Embodiment 76. An oligomeric compound comprising a modified oligonucleotide consisting of 12 to 30 linked nucleosides wherein the nucleobase sequence of the modified oligonucleotide is complementary to at least 8, at least 9, at least 10, at least 11, at least 12, at least 13, at least 14, at least 15, at least 16, at least 17, at least 18, at least 19, at least 20, or at least 21 contiguous nucleobases of:

an equal length portion of nucleobases 19600-19642 of SEQ ID NO: 2; or

an equal length portion of nucleobases 19640-19672 of SEQ ID NO: 2.

Embodiment 77. An oligomeric compound comprising a modified oligonucleotide consisting of 12 to 30 linked nucleosides and having a nucleobase sequence comprising at least 8, at least 9, at least 10, at least 11, at least 12, at least 13, at least 14, at least 15, at least 16, at least 17, at least 18, at least 19, at least 20, at least 21, at least 22 or at least 23 contiguous nucleobases of:

SEQ ID NOS: 2670, 2582, and 2677; or

SEQ ID NOS: 2609, 2606, and 2578.

Embodiment 78. The oligomeric compound of any of embodiments 75-77, wherein the oligomeric compound comprises an antisense RNAi oligonucleotide comprising a targeting region comprising at least 15 contiguous nucleobases, wherein the targeting region is at least 90% complementary to an equal-length portion of a SPDEF RNA.
Embodiment 79. The oligomeric compound of embodiment 78, wherein the targeting region of the antisense RNAi oligonucleotide is at least 95% complementary or is 100% complementary to the equal length portion of a SPDEF RNA.
Embodiment 80. The oligomeric compound of any of embodiments 78 or 79, wherein the targeting region of the antisense RNAi oligonucleotide comprises at least 19, 20, 21, or 25 contiguous nucleobases.
Embodiment 81. The oligomeric compound of any of embodiments 78-80, wherein the SPDEF RNA has the nucleobase sequence of any of SEQ ID NOs: 1-6.
Embodiment 82. The oligomeric compound of any of embodiments 78-81 wherein at least one nucleoside of the antisense RNAi oligonucleotide comprises a modified sugar moiety selected from: 2′-F, 2′-OMe, 2′-NMA, LNA, and cEt; or a sugar surrogate selected from GNA, and UNA.
Embodiment 83. The oligomeric compound of any of embodiments 78-82, wherein each nucleoside of the antisense RNAi oligonucleotide comprises a modified sugar moiety or a sugar surrogate.
Embodiment 84. The oligomeric compound of any of embodiments 78-83 wherein at least 80%, at least 90%, or 100% of the nucleosides of the antisense RNAi oligonucleotide comprises a modified sugar moiety selected from 2′-F and 2′-OMe.
Embodiment 85. The oligomeric compound of any of embodiments 78-84, comprising a stabilized phosphate group attached to the 5′ position of the 5′-most nucleoside of the antisense RNAi oligonucleotide.
Embodiment 86. The oligomeric compound of embodiment 85, wherein the stabilized phosphate group comprises a cyclopropyl phosphonate or an (E)-vinyl phosphonate.
Embodiment 87. The oligomeric compound of any of embodiments 78-86, consisting of the RNAi antisense oligonucleotide.
Embodiment 88. The oligomeric compound of any of embodiments 78-87, comprising a conjugate group comprising a conjugate moiety and a conjugate linker.
Embodiment 89. The oligomeric compound of embodiment 88, wherein the conjugate linker consists of a single bond.
Embodiment 90. The oligomeric compound of embodiment 88, wherein the conjugate linker is cleavable.
Embodiment 91. The oligomeric compound of embodiment 88, wherein the conjugate linker comprises 1-3 linker-nucleosides.
Embodiment 92. The oligomeric compound of any of embodiments 88-91, wherein the conjugate group is attached to the 5′-end of the antisense RNAi oligonucleotide.
Embodiment 93. The oligomeric compound of any of embodiments 88-91, wherein the conjugate group is attached to the 3′-end of the antisense RNAi oligonucleotide.
Embodiment 94. The oligomeric compound of any of embodiments 78-93, comprising a terminal group.
Embodiment 95. The oligomeric compound of any of embodiments 75-90, 92 and 93, wherein the oligomeric compound does not comprise linker-nucleosides.
Embodiment 96. An oligomeric duplex comprising the oligomeric compound of any one of embodiments 75-95.
Embodiment 97. The oligomeric duplex of embodiment 96, wherein the oligomeric complex is an RNAi compound.
Embodiment 98. The oligomeric duplex of embodiment 96 or 97, comprising a sense RNAi oligonucleotide consisting of 17 to 30 linked nucleosides, wherein the nucleobase sequence of the sense RNAi oligonucleotide comprises an antisense-hybridizing region comprising least 15 contiguous nucleobases wherein the antisense-hybridizing region is at least 90% complementary to an equal length portion of the antisense RNAi oligonucleotide.
Embodiment 99. The oligomeric duplex of embodiment 98, wherein the sense RNAi oligonucleotide consists of 18-25, 20-25, or 21-23 linked nucleosides.
Embodiment 100. The oligomeric duplex of embodiment 98, wherein the sense RNAi oligonucleotide consists of 21 or 23 linked nucleosides.
Embodiment 101. The oligomeric duplex of any of embodiments 98-100, wherein 1-4 3′-most nucleosides of the antisense or the sense RNAi oligonucleotide are overhanging nucleosides.
Embodiment 102. The oligomeric duplex of any of embodiments 98-101, wherein 1-4 5′-most nucleosides of the antisense or sense RNAi oligonucleotide are overhanging nucleosides.
Embodiment 103. The oligomeric duplex of any of embodiments 98-102, wherein the duplex is blunt ended at the 3′-end of the antisense RNAi oligonucleotide.
Embodiment 104. The oligomeric duplex of any of embodiments 98-103, wherein the duplex is blunt ended at the 5′-end of the antisense RNAi oligonucleotide.
Embodiment 105. The oligomeric duplex of any of embodiments 98-104, wherein at least one nucleoside of the sense RNAi oligonucleotide comprises a modified sugar moiety selected from: 2′-F, 2′-OMe, LNA, cEt, or a sugar surrogate selected from GNA, and UNA.
Embodiment 106. The oligomeric duplex of embodiment 105, wherein each nucleoside of the sense RNAi oligonucleotide comprises a modified sugar moiety or a sugar surrogate.
Embodiment 107. The oligomeric duplex of embodiment 105, wherein at least 80%, at least 90%, or 100% of the nucleosides of the sense RNAi oligonucleotide comprises a modified sugar moiety selected from 2′-F and 2′-OMe.
Embodiment 108. The oligomeric duplex of any of embodiments 98-107, wherein at least one nucleoside of the sense RNAi oligonucleotide comprises a modified nucleobase.
Embodiment 109. The oligomeric duplex of any of embodiments 98-108, wherein at least one internucleoside linkage of the sense RNAi oligonucleotide is a modified internucleoside linkage.
Embodiment 110. The oligomeric duplex of embodiment 109, wherein at least one internucleoside linkage of the sense RNAi oligonucleotide is a phosphorothioate internucleoside linkage.
Embodiment 111. The oligomeric duplex of any of embodiments 98-110, wherein the compound comprises 1-5 abasic sugar moieties attached to one or both ends of the antisense or sense RNA oligonucleotide.
Embodiment 112. The oligomeric duplex of embodiment 111, wherein the antisense RNAi oligonucleotide has a nucleobase sequence comprising the nucleobase sequence of any of SEQ ID NOs: 2324-2510; wherein the sense RNAi oligonucleotide has a nucleobase sequence comprising the corresponding complementary nucleobase sequence of any of SEQ ID NOs: 2511-2697; and wherein the nucleobase sequence of the sense RNAi oligonucleotide is 100% complementary to the nucleobase sequence of the antisense RNAi oligonucleotide.
Embodiment 113. The oligomeric duplex of any of embodiments 98-102, consisting of the antisense RNAi oligonucleotide and the sense RNAi oligonucleotide.
Embodiment 114. The oligomeric duplex of any of embodiments 98-113, wherein the second oligomeric compound comprises a conjugate group comprising a conjugate moiety and a conjugate linker.
Embodiment 115. The oligomeric duplex of embodiment 114, wherein the conjugate linker consists of a single bond.
Embodiment 116. The oligomeric duplex of embodiment 115, wherein the conjugate linker is cleavable.
Embodiment 117. The oligomeric duplex of embodiment 115 or 116, wherein the conjugate linker comprises 1-3 linker-nucleosides.
Embodiment 118. The oligomeric duplex of any of embodiments 114-117, wherein the conjugate group is attached to the 5′-end of the sense RNAi oligonucleotide.
Embodiment 119. The oligomeric duplex of any of embodiments 114-117, wherein the conjugate group is attached to the 3′-end of the sense RNAi oligonucleotide.
Embodiment 120. The oligomeric duplex of any of embodiments 114-119, wherein the conjugate group is attached via the 2′ position of a ribosyl sugar moiety at an internal position of the sense RNAi oligonucleotide.
Embodiment 121. The oligomeric duplex of any one of embodiment 98-120, wherein the second oligomeric compound comprises a terminal group.
Embodiment 122. A pharmaceutical composition comprising the oligomeric compound of any one of embodiments 75-95 or the oligomeric duplex of any one of embodiments 96-121; and a pharmaceutically acceptable carrier or diluent.
Embodiment 123. The pharmaceutical composition of embodiment 122, wherein the pharmaceutically acceptable diluent is water, sterile saline, or PBS.
Embodiment 124. The pharmaceutical composition of embodiment 123, wherein the pharmaceutical composition consists essentially of the oligomeric duplex and sterile saline.
Embodiment 125. A method comprising administering to a subject a pharmaceutical composition of any of embodiments 122-124.
Embodiment 126. A method of treating a disease associated with SPDEF comprising administering to a subject having or at risk for developing a disease associated with SPDEF a therapeutically effective amount of the oligomeric compound of any one of embodiments 75-95, the oligomeric duplex of any one of embodiments 93-118, or the pharmaceutical composition of any one of embodiments 122-124, thereby treating the disease associated with SPDEF.
Embodiment 127. The method of embodiment 126, wherein the disease associated with SPDEF is selected from bronchitis, asthma, chronic obstructive pulmonary disease, pulmonary fibrosis, idiopathic pulmonary fibrosis, pneumonia, emphysema, rhinitis, sinusitis, nasal polyposis, sinus polyposis, bronchiectasis, and sarcoidosis.
Embodiment 128. The method of any of embodiments 126 or 127, wherein at least one symptom or hallmark of the disease associated with SPDEF is ameliorated.
Embodiment 129. The method of embodiment 128, wherein the symptom or hallmark is shortness of breath, chest pain, coughing, wheezing, fatigue, and sleep disruption.
Embodiment 130. The method of embodiment 126, wherein the disease associated with SPDEF is ulcerative colitis.
Embodiment 131. Use of the oligomeric compound of any one of embodiments 78-98, the oligomeric duplex of any one of embodiments 96-121, or the pharmaceutical composition of any one of embodiments 122-124 for the treatment of a pulmonary condition.
Embodiment 132. The use of embodiment 131, wherein the pulmonary condition is selected from bronchitis, asthma, chronic obstructive pulmonary disease, pneumonia, emphysema, rhinitis, sinusitis, nasal polyposis, sinus polyposis, bronchiectasis, and sarcoidosis.
Embodiment 133. The use of embodiment 131, wherein the pulmonary condition is chronic bronchitis.
Embodiment 134. The use of embodiment 131, wherein the pulmonary condition is severe asthma.
Embodiment 135. A method of reducing mucus production in the gastrointestinal tract of a subject, the method comprising administering the oligomeric compound of any one of embodiments 75-95, the oligomeric duplex of any one of embodiments 96-121, or the pharmaceutical composition of any one of embodiments 122-124.
Embodiment 136. A method of treating a gastrointestinal condition comprising administering to a subject having or at risk for developing the gastrointestinal condition a therapeutically effective amount of the oligomeric compound of any one of embodiments 75-95, the oligomeric duplex of any one of embodiments 96-121, or the pharmaceutical composition of any one of embodiments 122-124, thereby treating the gastrointestinal condition.
Embodiment 137. A method of reducing SPDEF RNA or SPDEF protein in the gastrointestinal tract of a subject having or at risk for developing a gastrointestinal condition, the method comprising administering a therapeutically effective amount of the oligomeric compound of any one of embodiments 75-95, the oligomeric duplex of any one of embodiments 96-121, or the pharmaceutical composition of any one of embodiments 122-124, thereby reducing SPDEF RNA or SPDEF protein in the gastrointestinal tract.
Embodiment 138. The method of embodiment 136 or 137, wherein the gastrointestinal condition is ulcerative colitis.
Embodiment 139. A method of reducing inflammation in a subject in need thereof, the method comprising administering to the subject the oligomeric compound of any one of embodiments 1-36 and 75-95; the oligomeric duplex of any one of embodiments 37 and 96-121; the antisense compound of embodiment 38; the modified oligonucleotide of any one of embodiments 39-46; or the pharmaceutical composition of any one of embodiments 47-49 and 122-124, thereby reducing inflammation in the subject.
Embodiment 140. A method of reducing inflammation in a lung of a subject in need thereof, the method comprising administering to the subject the oligomeric compound of any one of embodiments 1-36 and 75-95; the oligomeric duplex of any one of embodiments 37 and 96-121; the antisense compound of embodiment 38; the modified oligonucleotide of any one of embodiments 39-46; or the pharmaceutical composition of any one of embodiments 47-49 and 122-124, thereby reducing inflammation in the lung of the subject.
Embodiment 141. A method of reducing inflammation in the gastrointestinal tract of a subject in need thereof, the method comprising administering to the subject the oligomeric compound of any one of embodiments 1-36 and 75-95; the oligomeric duplex of any one of embodiments 37 and 96-121; the antisense compound of embodiment 38; the modified oligonucleotide of any one of embodiments 39-46; or the pharmaceutical composition of any one of embodiments 47-49 and 122-124, thereby reducing inflammation of the gastrointestinal tract of the subject.

### Certain Compounds

Certain embodiments provide compounds targeted to a SPDEF nucleic acid. In certain embodiments, the SPDEF nucleic acid has the sequence set forth in RefSeq or GENBANK Accession No. GENBANK Accession No. NM_012391.2 (SEQ ID NO: 1), the complement of GENBANK Accession No. NC_000006.12 truncated from nucleotides 34536001 to 34558000 (SEQ ID NO: 2), GENBANK Accession No. NM_001252294.1 (SEQ ID NO: 3), GENBANK Accession No. XM_005248988.3 (SEQ ID NO: 4), or GENBANK Accession No. XM_006715048.1 (SEQ ID NO: 5), each of which is incorporated by reference in its entirety. In certain embodiments, the compound is an antisense compound or oligomeric compound. In certain embodiments, the compound is single-stranded.

Certain embodiments provide a compound comprising a modified oligonucleotide consisting of 8 to 80 linked nucleosides and having a nucleobase sequence comprising at least 8 contiguous nucleobases of any of the nucleobase sequences of SEQ ID NOs: 15-2284. In certain embodiments, the compound is an antisense compound or oligomeric compound. In certain embodiments, the compound is single-stranded. In certain embodiments, the modified oligonucleotide consists of 10 to 30 linked nucleosides.

Certain embodiments provide a compound comprising a modified oligonucleotide consisting of 9 to 80 linked nucleosides and having a nucleobase sequence comprising at least 9 contiguous nucleobases of any of the nucleobase sequences of SEQ ID NOs: 15-2284. In certain embodiments, the compound is an antisense compound or oligomeric compound. In certain embodiments, the compound is single-stranded. In certain embodiments, the modified oligonucleotide consists of 10 to 30 linked nucleosides.

Certain embodiments provide a compound comprising a modified oligonucleotide consisting of 10 to 80 linked nucleosides and having a nucleobase sequence comprising at least 10 contiguous nucleobases of any of the nucleobase sequences of SEQ ID NOs: 15-2284. In certain embodiments, the compound is an antisense compound or oligomeric compound. In certain embodiments, the compound is single-stranded. In certain embodiments, the modified oligonucleotide consists of 10 to 30 linked nucleosides.

Certain embodiments provide a compound comprising a modified oligonucleotide consisting of 11 to 80 linked nucleosides and having a nucleobase sequence comprising at least 11 contiguous nucleobases of any of the nucleobase sequences of SEQ ID NOs: 15-2284. In certain embodiments, the compound is an antisense compound or oligomeric compound. In certain embodiments, the compound is single-stranded. In certain embodiments, the modified oligonucleotide consists of 11 to 30 linked nucleosides.

Certain embodiments provide a compound comprising a modified oligonucleotide consisting of 12 to 80 linked nucleosides and having a nucleobase sequence comprising at least 12 contiguous nucleobases of any of the nucleobase sequences of SEQ ID NOs: 15-2284. In certain embodiments, the compound is an antisense compound or oligomeric compound. In certain embodiments, the compound is single-stranded. In certain embodiments, the modified oligonucleotide consists of 12 to 30 linked nucleosides.

Certain embodiments provide a compound comprising a modified oligonucleotide consisting of 16 to 80 linked nucleosides and having a nucleobase sequence comprising the nucleobase sequence of any one of SEQ ID NOs: 15-2284. In certain embodiments, the compound is an antisense compound or oligomeric compound. In certain embodiments, the compound is single-stranded. In certain embodiments, the modified oligonucleotide consists of 16 to 30 linked nucleosides.

Certain embodiments provide a compound comprising a modified oligonucleotide having a nucleobase sequence consisting of the nucleobase sequence of any one of SEQ ID NOs: 15-2284. In certain embodiments, the compound is an antisense compound or oligomeric compound. In certain embodiments, the compound is single-stranded.

In certain embodiments, a compound comprises a modified oligonucleotide consisting of 8 to 80 linked nucleosides and having at least an 8, 9, 10, 11, 12, 13, 14, 15, or 16 contiguous nucleobase portion complementary to an equal length portion within nucleotides 3531-3546, 9377-9392 9801-9816, 9802-9817, or 17492-17507 of SEQ ID NO: 2. In certain embodiments, the modified oligonucleotide consists of 10 to 30 linked nucleosides. In certain embodiments, the modified oligonucleotide consists of 16 to 30 linked nucleosides.

In certain embodiments, a compound comprises a modified oligonucleotide consisting of 8 to 80 linked nucleosides wherein the modified oligonucleotide is complementary within nucleotides 3531-3546, 9377-9392 9801-9816, 9802-9817, or 17492-17507 of SEQ ID NO: 2. In certain embodiments, the modified oligonucleotide consists of 10 to 30 linked nucleosides. In certain embodiments, the modified oligonucleotide consists of 16 to 30 linked nucleosides.

In certain embodiments, a compound comprises a modified oligonucleotide consisting of 8 to 80 linked nucleosides and having a nucleobase sequence comprising at least an 8, 9, 10, 11, 12, 13, 14, 15, or 16 contiguous nucleobase portion of the nucleobase sequence of any one of SEQ ID NOs: 1129, 1444, 761, 1983, or 2230. In certain embodiments, the modified oligonucleotide consists of 10 to 30 linked nucleosides.

In certain embodiments, the modified oligonucleotide consists of 16 to 30 linked nucleosides.

In certain embodiments, a compound comprises a modified oligonucleotide consisting of 16 to 80 linked nucleosides and having a nucleobase sequence comprising the nucleobase sequence of any one of SEQ ID NOs: 1129, 1444, 761, 1983, or 2230. In certain embodiments, the modified oligonucleotide consists of 16 to 30 linked nucleosides.

In certain embodiments, a compound comprises a modified oligonucleotide consisting of 16 linked nucleosides and having a nucleobase sequence consisting of any one of SEQ ID NOs: 1129, 1444, 761, 1983, or 2230.

In certain embodiments, any of the foregoing modified oligonucleotides has at least one modified internucleoside linkage, at least one modified sugar, and/or at least one modified nucleobase.

In certain embodiments, at least one nucleoside of any of the foregoing modified oligonucleotides comprises a modified sugar. In certain embodiments, the modified sugar comprises a 2′-O-methoxyethyl group. In certain embodiments, the modified sugar is a bicyclic sugar, such as a 4′-CH(CH₃)—O-2′ group, a 4′-CH₂—O-2′ group, or a 4′-(CH₂)2-O-2′ group.

In certain embodiments, at least one internucleoside linkage of the modified oligonucleotide comprises a modified internucleoside linkage, such as a phosphorothioate internucleoside linkage.

In certain embodiments, at least one nucleobase of any of the foregoing modified oligonucleotides is a modified nucleobase, such as 5-methylcytosine.

In certain embodiments, any of the foregoing modified oligonucleotides has:
a gap segment consisting of linked 2′-deoxynucleosides;
a 5′ wing segment consisting of linked nucleosides; and
a 3′ wing segment consisting of linked nucleosides;

wherein the gap segment is positioned between the 5′ wing segment and the 3′ wing segment and wherein each nucleoside of each wing segment comprises a modified sugar. In certain embodiments, the modified oligonucleotide consists of 16 to 80 linked nucleosides and has a nucleobase sequence comprising the nucleobase sequence recited in any one of SEQ ID NOs: 1129, 1444, 761, 1983, or 2230. In certain embodiments, the modified oligonucleotide consists of 16 to 30 linked nucleosides and has a nucleobase sequence comprising the nucleobase sequence recited in any one of SEQ ID NOs: 1129, 1444, 761, 1983, or 2230. In certain embodiments, the modified oligonucleotide consists of 16 linked nucleosides and has a nucleobase sequence consisting of the nucleobase sequence recited in any one of SEQ ID NOs: 1129, 1444, 761, 1983, or 2230.

In certain embodiments, a compound comprises or consists of a modified oligonucleotide consisting of 16 to 80 linked nucleobases and having a nucleobase sequence comprising the nucleobase sequence of any one of SEQ ID NOs: 15-2284, wherein the modified oligonucleotide has:
a gap segment consisting of linked 2′-deoxynucleosides;
a 5′ wing segment consisting of linked nucleosides; and
a 3′ wing segment consisting of linked nucleosides;

wherein the gap segment is positioned between the 5′ wing segment and the 3′ wing segment and wherein each nucleoside of each wing segment comprises a modified sugar. In certain embodiments, the modified oligonucleotide consists of 16 to 30 linked nucleosides. In certain embodiments, the modified oligonucleotide consists of 16 linked nucleosides.

In certain embodiments, a compound comprises or consists of a modified oligonucleotide consisting of 16 to 80 linked nucleobases and having a nucleobase sequence comprising the nucleobase sequence recited in any one of SEQ ID NOs: 1129, 1444, 761, 1983, or 2230, wherein the modified oligonucleotide has:
a gap segment consisting of linked 2′-deoxynucleosides;
a 5′ wing segment consisting of linked nucleosides; and
a 3′ wing segment consisting of linked nucleosides;

wherein the gap segment is positioned between the 5′ wing segment and the 3′ wing segment and wherein each nucleoside of each wing segment comprises a modified sugar. In certain embodiments, the modified oligonucleotide consists of 16 to 30 linked nucleosides. In certain embodiments, the modified oligonucleotide consists of 16 linked nucleosides.

In certain embodiments, a compound comprises or consists of a modified oligonucleotide consisting of 16 to 80 linked nucleobases and having a nucleobase sequence comprising the nucleobase sequence recited in any one of SEQ ID NOs: 1129, 1444, or 761, wherein the modified oligonucleotide has:

a gap segment consisting often linked 2′-deoxynucleosides;

a 5′ wing segment consisting of three linked nucleosides; and

a 3′ wing segment consisting of three linked nucleosides;

wherein the gap segment is positioned between the 5′ wing segment and the 3′ wing segment; wherein each nucleoside of each wing segment comprises a cEt nucleoside; wherein each internucleoside linkage is a phosphorothioate linkage; and wherein each cytosine is a 5-methylcytosine. In certain embodiments, the modified oligonucleotide consists of 16 to 30 linked nucleosides. In certain embodiments, the modified oligonucleotide consists of 16 linked nucleosides.

In certain embodiments, a compound comprises or consists of a modified oligonucleotide consisting of 16 to 80 linked nucleobases and having a nucleobase sequence comprising the nucleobase sequence recited in any one of SEQ ID NOs: 1983 or 2230, wherein the modified oligonucleotide comprises:

a gap segment consisting of nine linked 2′-deoxynucleosides;

a 5′ wing segment consisting of two linked nucleosides; and

a 3′ wing segment consisting of five linked nucleosides;

wherein the gap segment is positioned between the 5′ wing segment and the 3′ wing segment; wherein each nucleoside of the 5′ wing segment comprises a cEt nucleoside; wherein the 3′ wing segment comprises a 2′-O-methoxyethyl nucleoside, a 2′-O-methoxyethyl nucleoside, a 2′-O-methoxyethyl nucleoside, a cEt nucleoside, and a cEt nucleoside in the 5′ to 3′ direction; wherein each internucleoside linkage is a phosphorothioate linkage; and wherein each cytosine is a 5-methylcytosine. In certain embodiments, the modified oligonucleotide consists of 16 to 30 linked nucleosides. In certain embodiments, the modified oligonucleotide consists of 16 linked nucleosides. Certain methods

Certain embodiments provided herein relate to methods of inhibiting SPDEF expression, which can be useful for treating, preventing, or ameliorating a disease associated with SPDEF in a subject, by administration of a compound that targets a SPDEF nucleic acid. In certain embodiments, the compound can be a SPDEF specific inhibitor. In certain embodiments, the compound can be an antisense compound, oligomeric compound, or oligonucleotide targeted to a SPDEF nucleic acid.

Examples of diseases associated with SPDEF treatable, preventable, and/or ameliorable with the compounds and methods provided herein include bronchitis, asthma, COPD, pulmonary fibrosis, idiopathic pulmonary fibrosis (IPF), pneumonia, emphysema, rhinitis, sinusitis, nasal polyposis, sinus polyposis, bronchiectasis, or sarcoidosis.

In certain embodiments, methods comprise administering a compound comprising a SPDEF specific inhibitor to a subject. In certain embodiments, the subject has a disease associated with SPDEF. In certain embodiments, the subject has bronchitis, asthma, COPD, pulmonary fibrosis, idiopathic pulmonary fibrosis (IPF), pneumonia, emphysema, rhinitis, sinusitis, nasal polyposis, sinus polyposis, bronchiectasis, or sarcoidosis. In certain embodiments, the disease is asthma. In certain embodiments, the disease is IPF. In certain embodiments, the disease comprises inflammation. In certain embodiments, the disease comprises inflammation in a lung of the subject. In certain embodiments, the disease comprises inflammation in the gastrointestinal tract of the subject. In certain embodiments, the compound comprises an antisense compound targeted to a SPDEF nucleic acid. In certain embodiments, the compound comprises an oligonucleotide targeted to a SPDEF nucleic acid. In certain embodiments, the compound comprises a modified oligonucleotide consisting of 8 to 80 linked nucleosides and having a nucleobase sequence comprising at least 8 contiguous nucleobases of any of the nucleobase sequences of SEQ ID NOs: 15-2284. In certain embodiments, the compound comprises a modified oligonucleotide consisting of 16 to 80 linked nucleosides and having a nucleobase sequence comprising the nucleobase sequence of any one of SEQ ID NOs: 15-2284. In certain embodiments, the compound comprises a modified oligonucleotide consisting of the nucleobase sequence of any one of SEQ ID NOs: 15-2284. In certain embodiments, the compound comprises a modified oligonucleotide of 16 to 80 linked nucleosides in length and having a nucleobase sequence comprising any one of SEQ ID NOs: 1129, 1444, 761, 1983, or 2230. In certain embodiments, the compound comprises a modified oligonucleotide having a nucleobase sequence consisting of any one of SEQ ID NOs: 1129, 1444, 761, 1983, or 2230. In any of the foregoing embodiments, the modified oligonucleotide can consist of 16 to 30 linked nucleosides. In certain embodiments, the compound is ION 833561, 833741, 833748, 936142, or 936158. In any of the foregoing embodiments, the compound can be an antisense compound or oligomeric compound. In certain embodiments, administering the compound reduces mucus production. In certain embodiments, administering the compound reduces lung fibrosis. In certain embodiments, administering the compound improves lung function.

In certain embodiments, methods of treating or ameliorating a disease associated with SPDEF comprise administering to the subject a compound comprising a SPDEF specific inhibitor, thereby treating or ameliorating the disease. In certain embodiments, the disease is bronchitis, asthma, COPD, pulmonary fibrosis, idiopathic pulmonary fibrosis (IPF), pneumonia, emphysema, rhinitis, sinusitis, nasal polyposis, sinus polyposis, bronchiectasis, or sarcoidosis. In certain embodiments, the disease is asthma. In certain embodiments, the disease is IPF. In certain embodiments, the disease comprises inflammation. In certain embodiments, the disease comprises inflammation in a lung of the subject. In certain embodiments, the disease comprises inflammation in the gastrointestinal tract of the subject. In certain embodiments, the compound comprises an antisense compound targeted to a SPDEF nucleic acid. In certain embodiments, the compound comprises an oligonucleotide targeted to a SPDEF nucleic acid. In certain embodiments, the compound comprises a modified oligonucleotide consisting of 8 to 80 linked nucleosides and having a nucleobase sequence comprising at least 8 contiguous nucleobases of any of the nucleobase sequences of SEQ ID NOs: 15-2284. In certain embodiments, the compound comprises a modified oligonucleotide consisting of 16 to 80 linked nucleosides and having a nucleobase sequence comprising the nucleobase sequence of any one of SEQ ID NOs: 15-2284. In certain embodiments, the compound comprises a modified oligonucleotide consisting of the nucleobase sequence of any one of SEQ ID NOs: 15-2284. In certain embodiments, the compound comprises a modified oligonucleotide of 16 to 80 linked nucleosides in length and having a nucleobase sequence comprising any one of SEQ ID NOs: 1129, 1444, 761, 1983, or 2230. In certain embodiments, the compound comprises a modified oligonucleotide having a nucleobase sequence consisting of any one of SEQ ID NOs: 1129, 1444, 761, 1983, or 2230. In any of the foregoing embodiments, the modified oligonucleotide can consist of 16 to 30 linked nucleosides. In certain embodiments, the compound is ION 833561, 833741, 833748, 936142, or 936158. In any of the foregoing embodiments, the compound can be an antisense compound or oligomeric compound. In certain embodiments, administering the compound reduces mucus production. In certain embodiments, administering the compound reduces lung fibrosis. In certain embodiments, administering the compound improves lung function.

In certain embodiments, methods of inhibiting expression of SPDEF in a subject having, or at risk of having, a disease associated with SPDEF comprise administering to the subject a compound comprising a SPDEF specific inhibitor, thereby inhibiting expression of SPDEF in the subject. In certain embodiments, administering the compound inhibits expression of SPDEF in the lung. In certain embodiments, the subject has, or is at risk of having bronchitis, asthma, COPD, pulmonary fibrosis, idiopathic pulmonary fibrosis (IPF), pneumonia, emphysema, rhinitis, sinusitis, nasal polyposis, sinus polyposis, bronchiectasis, or sarcoidosis. In certain embodiments, the disease is asthma. In certain embodiments, the disease is IPF. In certain embodiments, the disease comprises inflammation. In certain embodiments, the disease comprises inflammation in a lung of the subject. In certain embodiments, the disease comprises inflammation in the gastrointestinal tract of the subject. In certain embodiments, the compound comprises an antisense compound targeted to a SPDEF nucleic acid. In certain embodiments, the compound comprises an oligonucleotide targeted to a SPDEF nucleic acid. In certain embodiments, the compound comprises a modified oligonucleotide consisting of 8 to 80 linked nucleosides and having a nucleobase sequence comprising at least 8 contiguous nucleobases of any of the nucleobase sequences of SEQ ID NOs: 15-2284. In certain embodiments, the compound comprises a modified oligonucleotide consisting of 16 to 80 linked nucleosides and having a nucleobase sequence comprising the nucleobase sequence of any one of SEQ ID NOs: 15-2284. In certain embodiments, the compound comprises a modified oligonucleotide consisting of the nucleobase sequence of any one of SEQ ID NOs: 15-2284. In certain embodiments, the compound comprises a modified oligonucleotide of 16 to 80 linked nucleosides in length and having a nucleobase sequence comprising any one of SEQ ID NOs: 1129, 1444, 761, 1983, or 2230. In certain embodiments, the compound comprises a modified oligonucleotide having a nucleobase sequence consisting of any one of SEQ ID NOs: 1129, 1444, 761, 1983, or 2230. In any of the foregoing embodiments, the modified oligonucleotide can consist of 16 to 30 linked nucleosides. In certain embodiments, the compound is ION 833561, 833741, 833748, 936142, or 936158. In any of the foregoing embodiments, the compound can be single-stranded. In any of the foregoing embodiments, the compound can be an antisense compound or oligomeric compound. In certain embodiments, the compound is administered to the subject parenterally. In certain embodiments, administering the compound reduces mucus production. In certain embodiments, administering the compound reduces lung fibrosis. In certain embodiments, administering the compound improves lung function. In certain embodiments, the subject is identified as having or at risk of having a disease associated with SPDEF.

In certain embodiments, methods of inhibiting expression of SPDEF in a cell comprise contacting the cell with a compound comprising a SPDEF specific inhibitor, thereby inhibiting expression of SPDEF in the cell. In certain embodiments, the cell is a lung cell. In certain embodiments, the cell is in the lung of a subject who has, or is at risk of having bronchitis, asthma, COPD, pulmonary fibrosis, idiopathic pulmonary fibrosis (IPF), pneumonia, emphysema, rhinitis, sinusitis, nasal polyposis, sinus polyposis, bronchiectasis, or sarcoidosis. In certain embodiments, the cell is in the lung of a subject who has asthma. In certain embodiments, the cell is in the lung of a subject who has IPF. In certain embodiments, the disease comprises inflammation. In certain embodiments, the disease comprises inflammation in a lung of the subject. In certain embodiments, the disease comprises inflammation in the gastrointestinal tract of the subject. In certain embodiments, the compound comprises an antisense compound targeted to a SPDEF nucleic acid. In certain embodiments, the compound comprises an oligonucleotide targeted to a SPDEF nucleic acid. In certain embodiments, the compound comprises a modified oligonucleotide consisting of 8 to 80 linked nucleosides and having a nucleobase sequence comprising at least 8 contiguous nucleobases of any of the nucleobase sequences of SEQ ID NOs: 15-2284. In certain embodiments, the compound comprises a modified oligonucleotide consisting of 16 to 80 linked nucleosides and having a nucleobase sequence comprising the nucleobase sequence of any one of SEQ ID NOs: 15-2284. In certain embodiments, the compound comprises a modified oligonucleotide consisting of the nucleobase sequence of any one of SEQ ID NOs: 15-2284. In certain embodiments, the compound comprises a modified oligonucleotide of 16 to 80 linked nucleosides in length and having a nucleobase sequence comprising any one of SEQ ID NOs: 1129, 1444, 761, 1983, or 2230. In certain embodiments, the compound comprises a modified oligonucleotide having a nucleobase sequence consisting of any one of SEQ ID NOs: 1129, 1444, 761, 1983, or 2230. In any of the foregoing embodiments, the modified oligonucleotide can consist of 16 to 30 linked nucleosides. In certain embodiments, the compound is ION 833561, 833741, 833748, 936142, or 936158. In any of the foregoing embodiments, the compound can be single-stranded. In any of the foregoing embodiments, the compound can be an antisense compound or oligomeric compound.

Certain embodiments are drawn to a compound comprising a SPDEF specific inhibitor for use in treating a disease associated with SPDEF. In certain embodiments, the disease is bronchitis, asthma, COPD, pulmonary fibrosis, idiopathic pulmonary fibrosis (IPF), pneumonia, emphysema, rhinitis, sinusitis, nasal polyposis, sinus polyposis, bronchiectasis, or sarcoidosis. In certain embodiments, the disease is asthma. In certain embodiments, the disease is IPF. In certain embodiments, the disease comprises inflammation. In certain embodiments, the disease comprises inflammation in a lung of the subject. In certain embodiments, the disease comprises inflammation in the gastrointestinal tract of the subject. In certain embodiments, the compound comprises an antisense compound targeted to a SPDEF nucleic acid. In certain embodiments, the compound comprises an oligonucleotide targeted to a SPDEF nucleic acid. In certain embodiments, the compound comprises a modified oligonucleotide consisting of 8 to 80 linked nucleosides and having a nucleobase sequence comprising at least 8 contiguous nucleobases of any of the nucleobase sequences of SEQ ID NOs: 15-2284. In certain embodiments, the compound comprises a modified oligonucleotide consisting of 16 to 80 linked nucleosides and having a nucleobase sequence comprising the nucleobase sequence of any one of SEQ ID NOs: 15-2284. In certain embodiments, the compound comprises a modified oligonucleotide consisting of the nucleobase sequence of any one of SEQ ID NOs: 15-2284. In certain embodiments, the compound comprises a modified oligonucleotide of 16 to 80 linked nucleosides in length and having a nucleobase sequence comprising any one of SEQ ID NOs: 1129, 1444, 761, 1983, or 2230. In certain embodiments, the compound comprises a modified oligonucleotide having a nucleobase sequence consisting of any one of SEQ ID NOs: 1129, 1444, 761, 1983, or 2230. In any of the foregoing embodiments, the modified oligonucleotide can consist of 16 to 30 linked nucleosides. In certain embodiments, the compound is ION 833561, 833741, 833748, 936142, or 936158. In any of the foregoing embodiments, the compound can be single-stranded. In any of the foregoing embodiments, the compound can be an antisense compound or oligomeric compound.

Certain embodiments are drawn to use of a compound comprising a SPDEF specific inhibitor for the manufacture or preparation of a medicament for treating a disease associated with SPDEF. In certain embodiments, the disease is bronchitis, asthma, COPD, pulmonary fibrosis, idiopathic pulmonary fibrosis (IPF), pneumonia, emphysema, rhinitis, sinusitis, nasal polyposis, sinus polyposis, bronchiectasis, or sarcoidosis. In certain embodiments, the disease is asthma. In certain embodiments, the disease is IPF. In certain embodiments, the disease comprises inflammation. In certain embodiments, the disease comprises inflammation in a lung of the subject. In certain embodiments, the disease comprises inflammation in the gastrointestinal tract of the subject. In certain embodiments, the compound comprises an antisense compound targeted to a SPDEF nucleic acid. In certain embodiments, the compound comprises an oligonucleotide targeted to a SPDEF nucleic acid. In certain embodiments, the compound comprises a modified oligonucleotide consisting of 8 to 80 linked nucleosides and having a nucleobase sequence comprising at least 8 contiguous nucleobases of any of the nucleobase sequences of SEQ ID NOs: 15-2284. In certain embodiments, the compound comprises a modified oligonucleotide consisting of 16 to 80 linked nucleosides and having a nucleobase sequence comprising the nucleobase sequence of any one of SEQ ID NOs: 15-2284. In certain embodiments, the compound comprises a modified oligonucleotide consisting of the nucleobase sequence of any one of SEQ ID NOs: 15-2284. In certain embodiments, the compound comprises a modified oligonucleotide of 16 to 80 linked nucleosides in length and having a nucleobase sequence comprising any one of SEQ ID NOs: 1129, 1444, 761, 1983, or 2230. In certain embodiments, the compound comprises a modified oligonucleotide having a nucleobase sequence consisting of any one of SEQ ID NOs: 1129, 1444, 761, 1983, or 2230. In any of the foregoing embodiments, the modified oligonucleotide can consist of 16 to 30 linked nucleosides. In certain embodiments, the compound is ION 833561, 833741, 833748, 936142, or 936158. In any of the foregoing embodiments, the compound can be single-stranded. In any of the foregoing embodiments, the compound can be an antisense compound or oligomeric compound.

In any of the foregoing methods or uses, the compound can be targeted to a SPDEF nucleic acid. In certain embodiments, the compound comprises or consists of a modified oligonucleotide, for example a modified oligonucleotide consisting of 8 to 80 linked nucleosides, 10 to 30 linked nucleosides, 12 to 30 linked nucleosides, or 16 linked nucleosides. In certain embodiments, the modified oligonucleotide is at least 80%, 85%, 90%, 95% or 100% complementary to any of the nucleobase sequences recited in SEQ ID NOs: 1-5. In certain embodiments, the modified oligonucleotide comprises at least one modified internucleoside linkage, at least one modified sugar and/or at least one modified nucleobase. In certain embodiments, the modified internucleoside linkage is a phosphorothioate internucleoside linkage, the modified sugar is a bicyclic sugar or a 2′-O-methoxyethyl, and the modified nucleobase is a 5-methylcytosine. In certain embodiments, the modified oligonucleotide comprises a gap segment consisting of linked 2′-deoxynucleosides; a 5′ wing segment consisting of linked nucleosides; and a 3′ wing segment consisting of linked nucleosides, wherein the gap segment is positioned immediately adjacent to and between the 5′ wing segment and the 3′ wing segment and wherein each nucleoside of each wing segment comprises a modified sugar.

In any of the foregoing methods or uses, the compound can comprise or consist of a modified oligonucleotide consisting of 16 to 80 linked nucleosides and having a nucleobase sequence comprising the nucleobase sequence of any one of SEQ ID NOs: 15-2284, wherein the modified oligonucleotide comprises:
a gap segment consisting of linked 2′-deoxynucleosides;
a 5′ wing segment consisting of linked nucleosides; and
a 3′ wing segment consisting of linked nucleosides;

wherein the gap segment is positioned between the 5′ wing segment and the 3′ wing segment and wherein each nucleoside of each wing segment comprises a modified sugar. In certain embodiments, the modified oligonucleotide consists of 16 to 30 linked nucleosides. In certain embodiments, the modified oligonucleotide consists of 16 linked nucleosides.

In any of the foregoing methods or uses, the compound can comprise or consist of a modified oligonucleotide consisting of 16 to 80 linked nucleobases and having a nucleobase sequence comprising the nucleobase sequence recited in any one of SEQ ID NOs: 1129, 1444, 761, 1983, or 2230, wherein the modified oligonucleotide comprises:
a gap segment consisting of linked 2′-deoxynucleosides;
a 5′ wing segment consisting of linked nucleosides; and
a 3′ wing segment consisting of linked nucleosides;

wherein the gap segment is positioned between the 5′ wing segment and the 3′ wing segment and wherein each nucleoside of each wing segment comprises a modified sugar. In certain embodiments, the modified oligonucleotide consists of 16 to 30 linked nucleosides. In certain embodiments, the modified oligonucleotide consists of 16 linked nucleosides.

In any of the foregoing methods or uses, the compound can comprise or consist of a modified oligonucleotide consisting of 16 to 80 linked nucleobases and having a nucleobase sequence comprising the nucleobase sequence recited in any one of SEQ ID NOs: 1129, 1444, or 761, wherein the modified oligonucleotide comprises:

a gap segment consisting often linked 2′-deoxynucleosides;

a 5′ wing segment consisting of three linked nucleosides; and

a 3′ wing segment consisting of three linked nucleosides;

wherein the gap segment is positioned between the 5′ wing segment and the 3′ wing segment; wherein each nucleoside of each wing segment comprises a cEt nucleoside; wherein each internucleoside linkage is a phosphorothioate linkage; and wherein each cytosine is a 5-methylcytosine. In certain embodiments, the modified oligonucleotide consists of 16 to 30 linked nucleosides. In certain embodiments, the modified oligonucleotide consists of 16 linked nucleosides.

In any of the foregoing methods or uses, the compound can comprise or consist of a modified oligonucleotide consisting of 16 to 80 linked nucleobases and having a nucleobase sequence comprising the nucleobase sequence recited in any one of SEQ ID NOs: 1983 or 2230, wherein the modified oligonucleotide comprises:

a gap segment consisting of nine linked 2′-deoxynucleosides;

a 5′ wing segment consisting of two linked nucleosides; and

a 3′ wing segment consisting of five linked nucleosides;

wherein the gap segment is positioned between the 5′ wing segment and the 3′ wing segment; wherein each nucleoside of the 5′ wing segment comprises a cEt nucleoside; wherein the 3′ wing segment comprises a 2′-O-methoxyethyl nucleoside, a 2′-O-methoxyethyl nucleoside, a 2′-O-methoxyethyl nucleoside, a cEt nucleoside, and a cEt nucleoside in the 5′ to 3′ direction; wherein each internucleoside linkage is a phosphorothioate linkage; and wherein each cytosine is a 5-methylcytosine. In certain embodiments, the modified oligonucleotide consists of 16 to 30 linked nucleosides. In certain embodiments, the modified oligonucleotide consists of 16 linked nucleosides.

I. Certain Oligonucleotides

In certain embodiments, provided herein are oligomeric compounds comprising oligonucleotides, which consist of linked nucleosides. Oligonucleotides may be unmodified oligonucleotides (RNA or DNA) or may be modified oligonucleotides. Modified oligonucleotides comprise at least one modification relative to unmodified RNA or DNA. That is, modified oligonucleotides comprise at least one modified nucleoside (comprising a modified sugar moiety and/or a modified nucleobase) and/or at least one modified internucleoside linkage.

A. Certain Modified Nucleosides

Modified nucleosides comprise a modified sugar moiety or a modified nucleobase or both a modified sugar moiety and a modified nucleobase.

1. Certain Sugar Moieties

In certain embodiments, modified sugar moieties are non-bicyclic modified sugar moieties. In certain embodiments, modified sugar moieties are bicyclic or tricyclic sugar moieties. In certain embodiments, modified sugar moieties are sugar surrogates. Such sugar surrogates may comprise one or more substitutions corresponding to those of other types of modified sugar moieties.

In certain embodiments, modified sugar moieties are non-bicyclic modified sugar moieties comprising a furanosyl ring with one or more substituent groups none of which bridges two atoms of the furanosyl ring to form a bicyclic structure. Such non-bridging substituents may be at any position of the furanosyl, including but not limited to substituents at the 2′, 4′, and/or 5′ positions. In certain embodiments one or more non-bridging substituent of non-bicyclic modified sugar moieties is branched. Examples of 2′-substituent groups suitable for non-bicyclic modified sugar moieties include but are not limited to: 2′-F, 2′—OCH₃ (“OMe” or “O-methyl”), and 2′-O(CH₂)₂OCH₃ (“MOE”). In certain embodiments, 2′-substituent groups are selected from among: halo, allyl, amino, azido, SH, CN, OCN, CF₃, OCF₃, O—C₁-C₁₀ alkoxy, O—C₁-C₁₀ substituted alkoxy, O—C₁-C₁₀ alkyl, O—C₁-C₁₀ substituted alkyl, S-alkyl, N(Rₘ)-alkyl, O-alkenyl, S-alkenyl, N(Rₘ)-alkenyl, O-alkynyl, S-alkynyl, N(Rₘ)-alkynyl, O-alkylenyl-O-alkyl, alkynyl, alkaryl, aralkyl, O-alkaryl, O-aralkyl, O(CH₂)₂SCH₃, O(CH₂)₂ON(Rₘ)(Rₙ) or OCH₂C(═O)—N(Rₘ)(R), where each Rₘ and Rₙ is, independently, H, an amino protecting group, or substituted or unsubstituted C₁-C₁₀ alkyl, and the 2′-substituent groups described in Cook et al., U.S. Pat. No. 6,531,584; Cook et al., U.S. Pat. No. 5,859,221; and Cook et al., U.S. Pat. No. 6,005,087. Certain embodiments of these 2′-substituent groups can be further substituted with one or more substituent groups independently selected from among: hydroxyl, amino, alkoxy, carboxy, benzyl, phenyl, nitro (NO₂), thiol, thioalkoxy, thioalkyl, halogen, alkyl, aryl, alkenyl and alkynyl. Examples of 4′-substituent groups suitable for non-bicyclic modified sugar moieties include but are not limited to alkoxy (e.g., methoxy), alkyl, and those described in Manoharan et al., WO 2015/106128. Examples of 5′-substituent groups suitable for non-bicyclic modified sugar moieties include but are not limited to: 5-methyl (R or S), 5′-vinyl, and 5′-methoxy. In certain embodiments, non-bicyclic modified sugar moieties comprise more than one non-bridging sugar substituent, for example, 2′-F-5′-methyl sugar moieties and the modified sugar moieties and modified nucleosides described in Migawa et al., WO 2008/101157 and Rajeev et al., US2013/0203836.).

In certain embodiments, a 2′-substituted non-bicyclic modified nucleoside comprises a sugar moiety comprising a non-bridging 2′-substituent group selected from: F, NH₂, N₃, OCF₃, OCH₃, O(CH₂)₃NH₂, CH₂CH═CH₂, OCH₂CH═CH₂, OCH₂CH₂OCH₃, O(CH₂)₂SCH₃, O(CH₂)₂ON(Rₘ)(Rₙ), O(CH₂)₂O(CH₂)₂N(CH₃)₂, and N-substituted acetamide (OCH₂C(═O)—N(Rₘ)(R)), where each Rₘ and Rₙ is, independently, H, an amino protecting group, or substituted or unsubstituted C₁-C₁₀ alkyl.

In certain embodiments, a 2′-substituted nucleoside non-bicyclic modified nucleoside comprises a sugar moiety comprising a non-bridging 2′-substituent group selected from: F, OCF₃, OCH₃, OCH₂CH₂OCH₃, O(CH₂)₂SCH₃, O(CH₂)₂ON(CH₃)₂, O(CH₂)₂O(CH₂)₂N(CH₃)₂, and OCH₂C(═O)—N(H)CH₃ (“NMA”).

In certain embodiments, a 2′-substituted non-bicyclic modified nucleoside comprises a sugar moiety comprising a non-bridging 2′-substituent group selected from: F, OCH₃, and OCH₂CH₂OCH₃.

Certain modified sugar moieties comprise a substituent that bridges two atoms of the furanosyl ring to form a second ring, resulting in a bicyclic sugar moiety. In certain such embodiments, the bicyclic sugar moiety comprises a bridge between the 4′ and the 2′ furanose ring atoms. Examples of such 4′ to 2′ bridging sugar substituents include but are not limited to: 4′-CH₂-2′, 4′-(CH₂)₂-2′, 4′-(CH₂)₃-2′, 4′-CH₂—O-2′ (“LNA”), 4′-CH₂—S-2′, 4′-(CH₂)₂—O-2′ (“ENA”), 4′-CH(CH₃)—O-2′ (referred to as “constrained ethyl” or “cEt”), 4′-CH₂—O—CH₂-2′, 4′-CH₂—N(R)-2′, 4′-CH(CH₂OCH₃)—O-2′ (“constrained MOE” or “cMOE”) and analogs thereof (see, e.g., Seth et al., U.S. Pat. No. 7,399,845, Bhat et al., U.S. Pat. No. 7,569,686, Swayze et al., U.S. Pat. No. 7,741,457, and Swayze et al., U.S. Pat. No. 8,022,193), 4′-C(CH₃)(CH₃)—O-2′ and analogs thereof (see, e.g., Seth et al., U.S. Pat. No. 8,278,283), 4′—CH₂—N(OCH₃)-2′ and analogs thereof (see, e.g., Prakash et al., U.S. Pat. No. 8,278,425), 4′-CH₂—O—N(CH₃)-2′ (see, e.g., Allerson et al., U.S. Pat. No. 7,696,345 and Allerson et al., U.S. Pat. No. 8,124,745), 4′-CH₂—C(H)(CH₃)-2′ (see, e.g., Zhou, et al., J. Org. Chem., 2009, 74, 118-134), 4′-CH₂—C(═CH₂)-2′ and analogs thereof (see e.g., Seth et al., U.S. Pat. No. 8,278,426), 4′-C(RₐR_{b})—N(R)—O-2′, 4′-C(RₐR_{b})—O—N(R)-2′, 4′-CH₂—O—N(R)-2′, and 4′-CH₂—N(R)—O- 2′, wherein each R, Rₐ, and R_{b} is, independently, H, a protecting group, or C₁-C₁₂ alkyl (see, e.g. Imanishi et al., U.S. Pat. No. 7,427,672).

In certain embodiments, such 4′ to 2′ bridges independently comprise from 1 to 4 linked groups independently selected from: —[C(Rₐ)(R_{b})]ₙ—, —[C(Rₐ)(R_{b})]ₙ—O—, —C(Rₐ)═C(R_{b})—, —C(Rₐ)═N—, —C(═NRₐ)—, —C(═O)—, —C(═S)—, —O—, —Si(Rₐ)₂—, —S(═O)ₓ—, and —N(Rₐ)—; wherein: x is 0, 1, or 2; n is 1, 2, 3, or 4; each Rₐ and R_{b} is, independently selected from: H, a protecting group, hydroxyl, C₁-C₁₂ alkyl, substituted C₁-C₁₂ alkyl, C₂-C₁₂ alkenyl, substituted C₂-C₁₂ alkenyl, C₂-C₁₂ alkynyl, substituted C₂-C₁₂ alkynyl, C₅-C₂₀ aryl, substituted C₅-C₂₀ aryl, heterocycle radical, substituted heterocycle radical, heteroaryl, substituted heteroaryl, C₅-C₇ alicyclic radical, substituted C₅-C₇ alicyclic radical, halogen, OJ₁, NJ₁J₂, SJ₁, N₃, COOJ₁, acyl (C(═O)—H), substituted acyl, CN, sulfonyl (S(═O)₂-J₁), and sulfoxyl (S(═O)-J₁); and each J₁ and J₂ is, independently selected from: H, C₁-C₁₂ alkyl, substituted C₁-C₁₂ alkyl, C₂-C₁₂ alkenyl, substituted C₂-C₁₂ alkenyl, C₂-C₁₂ alkynyl, substituted C₂-C₁₂ alkynyl, C₅-C₂₀ aryl, substituted C₅-C₂₀ aryl, acyl (C(═O)—H), substituted acyl, a heterocycle radical, a substituted heterocycle radical, C₁-C₁₂ aminoalkyl, substituted C₁-C₁₂ aminoalkyl, and a protecting group.

Additional bicyclic sugar moieties are known in the art, see, for example: Freier et al., Nucleic Acids Research, 1997, 25(22), 4429-4443, Albaek et al., J. Org. Chem., 2006, 71, 7731-7740, Singh et al., Chem. Commun., 1998, 4, 455-456; Koshkin et al., Tetrahedron, 1998, 54, 3607-3630; Kumar et al., Bioorg. Med. Chem. Lett., 1998, 8, 2219-2222; Singh et al., J. Org. Chem., 1998, 63, 10035-10039; Srivastava et al., J. Am. Chem. Soc., 2007, 129, 8362-8379; Wengel et al., U.S. Pat. No. 7,053,207; Imanishi et al., U.S. Pat. No. 6,268,490; Imanishi et al. U.S. Pat. No. 6,770,748; Imanishi et al., U.S. RE44,779; Wengel et al., U.S. Pat. No. 6,794,499; Wengel et al., U.S. Pat. No. 6,670,461; Wengel et al., U.S. Pat. No. 7,034,133; Wengel et al., U.S. Pat. No. 8,080,644; Wengel et al., U.S. Pat. No. 8,034,909; Wengel et al., U.S. Pat. No. 8,153,365; Wengel et al., U.S. Pat. No. 7,572,582; and Ramasamy et al., U.S. Pat. No. 6,525,191; Torsten et al., WO 2004/106356; Wengel et al., WO 1999/014226; Seth et al., WO 2007/134181; Seth et al., U.S. Pat. No. 7,547,684; Seth et al., U.S. Pat. No. 7,666,854; Seth et al., U.S. Pat. No. 8,088,746; Seth et al., U.S. Pat. No. 7,750,131; Seth et al., U.S. Pat. No. 8,030,467; Seth et al., U.S. Pat. No. 8,268,980; Seth et al., U.S. Pat. No. 8,546,556; Seth et al., U.S. Pat. No. 8,530,640; Migawa et al., U.S. Pat. No. 9,012,421; Seth et al., U.S. Pat. No. 8,501,805; and U.S. Patent Publication Nos. Allerson et al., US2008/0039618 and Migawa et al., US2015/0191727.

In certain embodiments, bicyclic sugar moieties and nucleosides incorporating such bicyclic sugar moieties are further defined by isomeric configuration. For example, an LNA nucleoside (described herein) may be in the α-L configuration or in the β-D configuration.

[chemical structure]

α-L-methyleneoxy (4′-CH₂—O-2′) or α-L-LNA bicyclic nucleosides have been incorporated into oligonucleotides that showed antisense activity (Frieden et al., Nucleic Acids Research, 2003, 21, 6365-6372). Herein, general descriptions of bicyclic nucleosides include both isomeric configurations. When the positions of specific bicyclic nucleosides (e.g., LNA or cEt) are identified in exemplified embodiments herein, they are in the β-D configuration, unless otherwise specified.

In certain embodiments, modified sugar moieties comprise one or more non-bridging sugar substituent and one or more bridging sugar substituent (e.g., 5′-substituted and 4′-2′ bridged sugars).

In certain embodiments, modified sugar moieties are sugar surrogates. In certain such embodiments, the oxygen atom of the sugar moiety is replaced, e.g., with a sulfur, carbon or nitrogen atom. In certain such embodiments, such modified sugar moieties also comprise bridging and/or non-bridging substituents as described herein. For example, certain sugar surrogates comprise a 4′-sulfur atom and a substitution at the 2′-position (see, e.g., Bhat et al., U.S. Pat. No. 7,875,733 and Bhat et al., U.S. Pat. No. 7,939,677) and/or the 5′ position.

In certain embodiments, sugar surrogates comprise rings having other than 5 atoms. For example, in certain embodiments, a sugar surrogate comprises a six-membered tetrahydropyran (“THP”). Such tetrahydropyrans may be further modified or substituted. Nucleosides comprising such modified tetrahydropyrans include but are not limited to hexitol nucleic acid (“HNA”), anitol nucleic acid (“ANA”), manitol nucleic acid (“MNA”) (see, e.g., Leumann, C J. Bioorg. & Med. Chem. 2002, 10, 841-854), fluoro HNA:

[chemical structure]

(“F-HNA”, see e.g. Swayze et al., U.S. Pat. No. 8,088,904; Swayze et al., U.S. Pat. No. 8,440,803; Swayze et al., U.S. Pat. No. 8,796,437; and Swayze et al., U.S. Pat. No. 9,005,906; F-HNA can also be referred to as a F-THP or 3′-fluoro tetrahydropyran), and nucleosides comprising additional modified THP compounds having the formula:

[chemical structure]

wherein, independently, for each of said modified THP nucleoside: Bx is a nucleobase moiety; T₃ and T₄ are each, independently, an internucleoside linking group linking the modified THP nucleoside to the remainder of an oligonucleotide or one of T₃ and T₄ is an internucleoside linking group linking the modified THP nucleoside to the remainder of an oligonucleotide and the other of T₃ and T₄ is H, a hydroxyl protecting group, a linked conjugate group, or a 5′ or 3′-terminal group; q₁, q₂, q₃, q₄, q₅, q₆ and q₇ are each, independently, H, C₁-C₆ alkyl, substituted C₁-C₆ alkyl, C₂-C₆ alkenyl, substituted C₂-C₆ alkenyl, C₂-C₆ alkynyl, or substituted C₂-C₆ alkynyl; and each of R₁ and R₂ is independently selected from among: hydrogen, halogen, substituted or unsubstituted alkoxy, NJ₁J₂, SJ₁, N₃, OC(═X)J₁, OC(═X)NJ₁J₂, NJ₃C(═X)NJ₁J₂, and CN, wherein X is O, S or NJ₁, and each J₁, J₂, and J₃ is, independently, H or C₁-C₆ alkyl.

In certain embodiments, modified THP nucleosides are provided wherein q₁, q₂, q₃, q₄, q₅, q₆ and q₇ are each H. In certain embodiments, at least one of q₁, q₂, q₃, q₄, q₅, q₆ and q₇ is other than H. In certain embodiments, at least one of q₁, q₂, q₃, q₄, q₅, q₆ and q₇ is methyl. In certain embodiments, modified THP nucleosides are provided wherein one of R₁ and R₂ is F. In certain embodiments, R₁ is F and R₂ is H, in certain embodiments, R₁ is methoxy and R₂ is H, and in certain embodiments, R₁ is methoxyethoxy and R₂ is H.

In certain embodiments, sugar surrogates comprise rings having more than 5 atoms and more than one heteroatom. For example, nucleosides comprising morpholino sugar moieties and their use in oligonucleotides have been reported (see, e.g., Braasch et al., Biochemistry, 2002, 41, 4503-4510 and Summerton et al., U.S. Pat. No. 5,698,685; Summerton et al., U.S. Pat. No. 5,166,315; Summerton et al., U.S. Pat. No. 5,185,444; and Summerton et al., U.S. Pat. No. 5,034,506). As used here, the term “morpholino” means a sugar surrogate having the following structure:

[chemical structure]

In certain embodiments, morpholinos may be modified, for example by adding or altering various substituent groups from the above morpholino structure. Such sugar surrogates are referred to herein as “modified morpholinos.”

In certain embodiments, sugar surrogates comprise acyclic moieties. Examples of nucleosides and oligonucleotides comprising such acyclic sugar surrogates include but are not limited to: peptide nucleic acid (“PNA”), acyclic butyl nucleic acid (see, e.g., Kumar et al., Org. Biomol. Chem., 2013, 11, 5853-5865), and nucleosides and oligonucleotides described in Manoharan et al., WO2011/133876.

Many other bicyclic and tricyclic sugar and sugar surrogate ring systems are known in the art that can be used in modified nucleosides.

2. Certain Modified Nucleobases

In certain embodiments, modified oligonucleotides comprise one or more nucleoside comprising an unmodified nucleobase. In certain embodiments, modified oligonucleotides comprise one or more nucleoside comprising a modified nucleobase. In certain embodiments, modified oligonucleotides comprise one or more nucleoside that does not comprise a nucleobase, referred to as an abasic nucleoside.

In certain embodiments, modified nucleobases are selected from: 5-substituted pyrimidines, 6-azapyrimidines, alkyl or alkynyl substituted pyrimidines, alkyl substituted purines, and N-2, N-6 and 0-6 substituted purines. In certain embodiments, modified nucleobases are selected from: 2-aminopropyladenine, 5-hydroxymethyl cytosine, xanthine, hypoxanthine, 2-aminoadenine, 6-N-methylguanine, 6-N-methyladenine, 2-propyladenine, 2-thiouracil, 2-thiothymine and 2-thiocytosine, 5-propynyl (—C═C—CH₃) uracil, 5-propynylcytosine, 6-azouracil, 6-azocytosine, 6-azothymine, 5-ribosyluracil (pseudouracil), 4-thiouracil, 8-halo, 8-amino, 8-thiol, 8-thioalkyl, 8-hydroxyl, 8-aza and other 8-substituted purines, 5-halo, particularly 5-bromo, 5-trifluoromethyl, 5-halouracil, and 5-halocytosine, 7-methylguanine, 7-methyladenine, 2-F-adenine, 2-aminoadenine, 7-deazaguanine, 7-deazaadenine, 3-deazaguanine, 3-deazaadenine, 6-N-benzoyladenine, 2-N-isobutyrylguanine, 4-N-benzoylcytosine, 4-N-benzoyluracil, 5-methyl 4-N-benzoylcytosine, 5-methyl 4-N-benzoyluracil, universal bases, hydrophobic bases, promiscuous bases, size-expanded bases, and fluorinated bases. Further modified nucleobases include tricyclic pyrimidines, such as 1,3-diazaphenoxazine-2-one, 1,3-diazaphenothiazine-2-one and 9-(2-aminoethoxy)-1,3-diazaphenoxazine-2-one (G-clamp). Modified nucleobases may also include those in which the purine or pyrimidine base is replaced with other heterocycles, for example 7-deaza-adenine, 7-deazaguanosine, 2-aminopyridine and 2-pyridone. Further nucleobases include those disclosed in Merigan et al., U.S. Pat. No. 3,687,808, those disclosed in The Concise Encyclopedia Of Polymer Science And Engineering, Kroschwitz, J. I., Ed., John Wiley & Sons, 1990, 858-859; Englisch et al., Angewandte Chemie, International Edition, 1991, 30, 613; Sanghvi, Y. S., Chapter 15, Antisense Research and Applications, Crooke, S. T. and Lebleu, B., Eds., CRC Press, 1993, 273-288; and those disclosed in Chapters 6 and 15, Antisense Drug Technology, Crooke S. T., Ed., CRC Press, 2008, 163-166 and 442-443.

Publications that teach the preparation of certain of the above noted modified nucleobases as well as other modified nucleobases include without limitation, Manoharan et al., US2003/0158403; Manoharan et al., US2003/0175906; Dinh et al., U.S. Pat. No. 4,845,205; Spielvogel et al., U.S. Pat. No. 5,130,302; Rogers et al., U.S. Pat. No. 5,134,066; Bischofberger et al., U.S. Pat. No. 5,175,273; Urdea et al., U.S. Pat. No. 5,367,066; Benner et al., U.S. Pat. No. 5,432,272; Matteucci et al., U.S. Pat. No. 5,434,257; Gmeiner et al., U.S. Pat. No. 5,457,187; Cook et al., U.S. Pat. No. 5,459,255; Froehler et al., U.S. Pat. No. 5,484,908; Matteucci et al., U.S. Pat. No. 5,502,177; Hawkins et al., U.S. Pat. No. 5,525,711; Haralambidis et al., U.S. Pat. No. 5,552,540; Cook et al., U.S. Pat. No. 5,587,469; Froehler et al., U.S. Pat. No. 5,594,121; Switzer et al., U.S. Pat. No. 5,596,091; Cook et al., U.S. Pat. No. 5,614,617; Froehler et al., U.S. Pat. No. 5,645,985; Cook et al., U.S. Pat. No. 5,681,941; Cook et al., U.S. Pat. No. 5,811,534; Cook et al., U.S. Pat. No. 5,750,692; Cook et al., U.S. Pat. No. 5,948,903; Cook et al., U.S. Pat. No. 5,587,470; Cook et al., U.S. Pat. No. 5,457,191; Matteucci et al., U.S. Pat. No. 5,763,588; Froehler et al., U.S. Pat. No. 5,830,653; Cook et al., U.S. Pat. No. 5,808,027; Cook et al., 6,166,199; and Matteucci et al., U.S. Pat. No. 6,005,096.

3. Certain Modified Internucleoside Linkages

In certain embodiments, nucleosides of modified oligonucleotides may be linked together using any internucleoside linkage. The two main classes of internucleoside linking groups are defined by the presence or absence of a phosphorus atom. Representative phosphorus-containing internucleoside linkages include but are not limited to phosphates, which contain a phosphodiester bond (“P═O”) (also referred to as unmodified or naturally occurring linkages), phosphotriesters, methylphosphonates, phosphoramidates, and phosphorothioates (“P═S”), and phosphorodithioates (“HS—P═S”). Representative non-phosphorus containing internucleoside linking groups include but are not limited to methylenemethylimino (—CH₂—N(CH₃)—O—CH₂—), thiodiester, thionocarbamate (—O—C(═O)(NH)—S—); siloxane (—O—SiH₂—O—); and N,N′-dimethylhydrazine (—CH₂—N(CH₃)—N(CH₃)—). Modified internucleoside linkages, compared to naturally occurring phosphate linkages, can be used to alter, typically increase, nuclease resistance of the oligonucleotide. In certain embodiments, internucleoside linkages having a chiral atom can be prepared as a racemic mixture, or as separate enantiomers. Methods of preparation of phosphorous-containing and non-phosphorous-containing internucleoside linkages are well known to those skilled in the art.

Representative internucleoside linkages having a chiral center include but are not limited to alkylphosphonates and phosphorothioates. Modified oligonucleotides comprising internucleoside linkages having a chiral center can be prepared as populations of modified oligonucleotides comprising stereorandom internucleoside linkages, or as populations of modified oligonucleotides comprising phosphorothioate linkages in particular stereochemical configurations. In certain embodiments, populations of modified oligonucleotides comprise phosphorothioate internucleoside linkages wherein all of the phosphorothioate internucleoside linkages are stereorandom. Such modified oligonucleotides can be generated using synthetic methods that result in random selection of the stereochemical configuration of each phosphorothioate linkage. Nonetheless, as is well understood by those of skill in the art, each individual phosphorothioate of each individual oligonucleotide molecule has a defined stereoconfiguration. In certain embodiments, populations of modified oligonucleotides are enriched for modified oligonucleotides comprising one or more particular phosphorothioate internucleoside linkages in a particular, independently selected stereochemical configuration. In certain embodiments, the particular configuration of the particular phosphorothioate linkage is present in at least 65% of the molecules in the population. In certain embodiments, the particular configuration of the particular phosphorothioate linkage is present in at least 70% of the molecules in the population. In certain embodiments, the particular configuration of the particular phosphorothioate linkage is present in at least 80% of the molecules in the population. In certain embodiments, the particular configuration of the particular phosphorothioate linkage is present in at least 90% of the molecules in the population. In certain embodiments, the particular configuration of the particular phosphorothioate linkage is present in at least 99% of the molecules in the population. Such chirally enriched populations of modified oligonucleotides can be generated using synthetic methods known in the art, e.g., methods described in Oka et al., JACS 125, 8307 (2003), Wan et al. Nuc. Acid. Res. 42, 13456 (2014), and WO 2017/015555. In certain embodiments, a population of modified oligonucleotides is enriched for modified oligonucleotides having at least one indicated phosphorothioate in the (Sp) configuration. In certain embodiments, a population of modified oligonucleotides is enriched for modified oligonucleotides having at least one phosphorothioate in the (Rp) configuration. In certain embodiments, modified oligonucleotides comprising (Rp) and/or (Sp) phosphorothioates comprise one or more of the following formulas, respectively, wherein “B” indicates a nucleobase:

[chemical structure]

Unless otherwise indicated, chiral internucleoside linkages of modified oligonucleotides described herein can be stereorandom or in a particular stereochemical configuration.

Neutral internucleoside linkages include, without limitation, phosphotriesters, methylphosphonates, MMI (3′-CH₂—N(CH₃)—O-5′), amide-3 (3′-CH₂—C(═O)—N(H)-5′), amide-4 (3′-CH₂—N(H)—C(═O)-5′), formacetal (3′-O—CH₂—O-5′), methoxypropyl, and thioformacetal (3′-S—CH₂—O-5′). Further neutral internucleoside linkages include nonionic linkages comprising siloxane (dialkylsiloxane), carboxylate ester, carboxamide, sulfide, sulfonate ester and amides (See for example: Carbohydrate Modifications in Antisense Research; Y. S. Sanghvi and P. D. Cook, Eds., ACS Symposium Series 580; Chapters 3 and 4, 40-65). Further neutral internucleoside linkages include nonionic linkages comprising mixed N, O, S and CH₂ component parts.

B. Certain Motifs

In certain embodiments, modified oligonucleotides comprise one or more modified nucleosides comprising a modified sugar moiety. In certain embodiments, modified oligonucleotides comprise one or more modified nucleosides comprising a modified nucleobase. In certain embodiments, modified oligonucleotides comprise one or more modified internucleoside linkage. In such embodiments, the modified, unmodified, and differently modified sugar moieties, nucleobases, and/or internucleoside linkages of a modified oligonucleotide define a pattern or motif. In certain embodiments, the patterns of sugar moieties, nucleobases, and internucleoside linkages are each independent of one another. Thus, a modified oligonucleotide may be described by its sugar motif, nucleobase motif and/or internucleoside linkage motif (as used herein, nucleobase motif describes the modifications to the nucleobases independent of the sequence of nucleobases).

1. Certain Sugar Motifs

In certain embodiments, oligonucleotides comprise one or more type of modified sugar and/or unmodified sugar moiety arranged along the oligonucleotide or region thereof in a defined pattern or sugar motif. In certain instances, such sugar motifs include but are not limited to any of the sugar modifications discussed herein.

### Uniformly Modified Oligonucleotides

In certain embodiments, modified oligonucleotides comprise or consist of a region having a fully modified sugar motif. In such embodiments, each nucleoside of the fully modified region of the modified oligonucleotide comprises a modified sugar moiety. In certain embodiments, each nucleoside of the entire modified oligonucleotide comprises a modified sugar moiety. In certain embodiments, modified oligonucleotides comprise or consist of a region having a fully modified sugar motif, wherein each nucleoside within the fully modified region comprises the same modified sugar moiety, referred to herein as a uniformly modified sugar motif. In certain embodiments, a fully modified oligonucleotide is a uniformly modified oligonucleotide.

### Gapmer Oligonucleotides

In certain embodiments, modified oligonucleotides comprise or consist of a region having a gapmer motif, which is defined by two external regions or “wings” and a central or internal region or “gap.” The three regions of a gapmer motif (the 5′-wing, the gap, and the 3′-wing) form a contiguous sequence of nucleosides wherein at least some of the sugar moieties of the nucleosides of each of the wings differ from at least some of the sugar moieties of the nucleosides of the gap. Specifically, at least the sugar moieties of the nucleosides of each wing that are closest to the gap (the 3′-most nucleoside of the 5′-wing and the 5′-most nucleoside of the 3′-wing) differ from the sugar moiety of the neighboring gap nucleosides, thus defining the boundary between the wings and the gap (i.e., the wing/gap junction). In certain embodiments, the sugar moieties within the gap are the same as one another. In certain embodiments, the gap includes one or more nucleoside having a sugar moiety that differs from the sugar moiety of one or more other nucleosides of the gap. In certain embodiments, the sugar motifs of the two wings are the same as one another (symmetric gapmer). In certain embodiments, the sugar motif of the 5′-wing differs from the sugar motif of the 3′-wing (asymmetric gapmer).

In certain embodiments, the wings of a gapmer comprise 1-5 nucleosides. In certain embodiments, each nucleoside of each wing of a gapmer is a modified nucleoside. In certain embodiments, at least one nucleoside of each wing of a gapmer is a modified nucleoside. In certain embodiments, at least two nucleosides of each wing of a gapmer are modified nucleosides. In certain embodiments, at least three nucleosides of each wing of a gapmer are modified nucleosides. In certain embodiments, at least four nucleosides of each wing of a gapmer are modified nucleosides.

In certain embodiments, the gap of a gapmer comprises 7-12 nucleosides. In certain embodiments, each nucleoside of the gap of a gapmer is an unmodified 2′-deoxynucleoside. In certain embodiments, at least one nucleoside of the gap of a gapmer is a modified nucleoside.

In certain embodiments, the gapmer is a deoxy gapmer. In certain embodiments, the nucleosides on the gap side of each wing/gap junction are unmodified 2′-deoxynucleosides and the nucleosides on the wing sides of each wing/gap junction are modified nucleosides. In certain embodiments, each nucleoside of the gap is an unmodified 2′-deoxynucleoside. In certain embodiments, each nucleoside of each wing of a gapmer is a modified nucleoside.

In certain embodiments, modified oligonucleotides comprise or consist of a region having a fully modified sugar motif. In such embodiments, each nucleoside of the fully modified region of the modified oligonucleotide comprises a modified sugar moiety. In certain embodiments, each nucleoside of the entire modified oligonucleotide comprises a modified sugar moiety. In certain embodiments, modified oligonucleotides comprise or consist of a region having a fully modified sugar motif, wherein each nucleoside within the fully modified region comprises the same modified sugar moiety, referred to herein as a uniformly modified sugar motif. In certain embodiments, a fully modified oligonucleotide is a uniformly modified oligonucleotide. In certain embodiments, each nucleoside of a uniformly modified comprises the same 2′-modification.

Herein, the lengths (number of nucleosides) of the three regions of a gapmer may be provided using the notation [#of nucleosides in the 5′-wing]-[#of nucleosides in the gap]-[#of nucleosides in the 3′-wing]. Thus, a 3-10-3 gapmer consists of 3 linked nucleosides in each wing and 10 linked nucleosides in the gap. Where such nomenclature is followed by a specific modification, that modification is the modification in each sugar moiety of each wing and the gap nucleosides comprise unmodified deoxynucleosides sugars. Thus, a 3-10-3 cEt gapmer consists of 3 linked cEt nucleosides in the 5′-wing, 10 linked deoxynucleosides in the gap, and 3 linked cEt nucleosides in the 3′-wing. Similarly, a 2-12-2 cEt gapmer consists of 2 linked cEt nucleosides in the 5′-wing, 12 linked deoxynucleosides in the gap, and 2 linked cEt nucleosides in the 3′-wing.

In certain embodiments, modified oligonucleotides are 3-10-3 BNA gapmers. In certain embodiments, modified oligonucleotides are 3-10-3 cEt gapmers. In certain embodiments, modified oligonucleotides are 3-10-3 LNA gapmers. In certain embodiments, modified oligonucleotides are 2-12-2 BNA gapmers. In certain embodiments, modified oligonucleotides are 2-12-2 cEt gapmers. In certain embodiments, modified oligonucleotides are 2-12-2 LNA gapmers.

### Antisense RNAi Oligonucleotides

In certain embodiments, the sugar moiety of at least one nucleoside of an antisense RNAi oligonucleotide is a modified sugar moiety.

In certain such embodiments, at least one nucleoside of the antisense RNAi oligonucleotide comprises a 2′-OMe modified sugar moiety. In certain embodiments, at least 2 nucleosides comprise 2′-OMe modified sugar moieties. In certain embodiments, at least 5 nucleosides comprise 2′-OMe modified sugar moieties. In certain embodiments, at least 8 nucleosides comprise 2′-OMe modified sugar moieties. In certain embodiments, at least 10 nucleosides comprise 2′-OMe modified sugar moieties. In certain embodiments, at least 12 nucleosides comprise 2′-OMe modified sugar moieties. In certain embodiments, at least 14 nucleosides comprise 2′-OMe modified sugar moieties. In certain embodiments, at least 15 nucleosides comprise 2′-OMe modified sugar moieties. In certain embodiments, at least 17 nucleosides comprise 2′-OMe modified sugar moieties. In certain such embodiments, at least 18 nucleosides comprise 2′-OMe modified sugar moieties. In certain such embodiments, at least 20 nucleosides comprise 2′-OMe modified sugar moieties. In certain embodiments, at least 21 nucleosides comprise 2′-OMe modified sugar moieties. In certain such embodiments, the remainder of the nucleosides are 2′-F modified.

In certain embodiments, at least one nucleoside of the antisense RNAi oligonucleotide comprises a 2′-F modified sugar moiety. In certain embodiments, at least 2 nucleosides comprise 2′-F modified sugar moieties. In certain embodiments, at least 3 nucleosides comprise 2′-F modified sugar moieties. In certain embodiments, at least 4 nucleosides comprise 2′-F modified sugar moieties. In certain embodiments, one, but not more than one nucleoside comprises a 2′-F modified sugar. In certain embodiments, 1 or 2 nucleosides comprise 2′-F modified sugar moieties. In certain embodiments, 1-3 nucleosides comprise 2′-F modified sugar moieties. In certain embodiments, at least 1-4 nucleosides comprise 2′-F modified sugar moieties. In certain embodiments, antisense RNAi oligonucleotides have a block of 2-4 contiguous 2′-F modified nucleosides. In certain embodiments, 4 nucleosides of an antisense RNAi oligonucleotide are 2′-F modified nucleosides and 3 of those 2′-F modified nucleosides are contiguous. In certain such embodiments, the remainder of the nucleosides are 2′-OMe modified.

In certain embodiments, at least one nucleoside of the antisense RNAi oligonucleotide comprises a 2′-OMe modified sugar moiety and at least one nucleoside comprises a 2′-F modified sugar moiety. In certain embodiments, at least 1, 2, 3, 4, 5, 6, 7, 8, 9 or 10 nucleosides comprises a 2′-OMe modified sugar moiety and at least 1, 2, 3, 4, 5, 6, 7, 8, 9 or 10 nucleosides comprises a 2′-F modified sugar moiety. In certain embodiments, the antisense RNAi oligonucleotide comprises a sugar motif of fyf or yfy, wherein each “f” represents a 2′-F modified sugar moiety and each “y” represents a 2′-OMe modified sugar moiety. In certain embodiments, the antisense RNAi oligonucleotide has a sugar motif of yfyfyfyfyfyfyfyfyfyfyyy, wherein each “f” represents a 2′-F modified sugar moiety and each “y” represents a 2′-OMe modified sugar moiety.

### Sense RNAi Oligonucleotides

In certain embodiments, the sugar moiety of at least one nucleoside of a sense RNAi oligonucleotides is a modified sugar moiety.

In certain such embodiments, at least one nucleoside of the sense RNAi oligonucleotide comprises a 2′-OMe modified sugar moiety. In certain embodiments, at least 2 nucleosides comprise 2′-OMe modified sugar moieties. In certain embodiments, at least 5 nucleosides comprise 2′-OMe modified sugar moieties. In certain embodiments, at least 8 nucleosides comprise 2′-OMe modified sugar moieties. In certain embodiments, at least 10 nucleosides comprise 2′-OMe modified sugar moieties. In certain embodiments, at least 12 nucleosides comprise 2′-OMe modified sugar moieties. In certain embodiments, at least 14 nucleosides comprise 2′-OMe modified sugar moieties. In certain embodiments, at least 15 nucleosides comprise 2′-OMe modified sugar moieties. In certain embodiments, at least 17 nucleosides comprise 2′-OMe modified sugar moieties. In certain such embodiments, at least 18 nucleosides comprise 2′-OMe modified sugar moieties. In certain such embodiments, at least 20 nucleosides comprise 2′-OMe modified sugar moieties. In certain such embodiments, at least 21 nucleosides comprise 2′-OMe modified sugar moieties.

In certain embodiments, at least one nucleoside of the sense RNAi oligonucleotide comprises a 2′-F modified sugar moiety. In certain embodiments, at least 2 nucleosides comprise 2′-F modified sugar moieties. In certain embodiments, at least 3 nucleosides comprise 2′-F modified sugar moieties. In certain embodiments, at least 4 nucleosides comprise 2′-F modified sugar moieties. In certain embodiments, one, but not more than nucleoside comprises a 2′-F modified sugar moiety. In certain embodiments, 1 or 2 nucleosides comprise 2′-F modified sugar moieties. In certain embodiments, 1-3 nucleosides comprise 2′-F modified sugar moieties. In certain embodiments, at least 1-4 nucleosides comprise 2′-F modified sugar moieties. In certain embodiments, sense RNAi oligonucleotides have a block of 2-4 contiguous 2′-F modified nucleosides. In certain embodiments, 4 nucleosides of a sense RNAi oligonucleotide are 2′-F modified nucleosides and 3 of those 2′-F modified nucleosides are contiguous. In certain such embodiments the remainder of the nucleosides are 2′OMe modified.

In certain embodiments, at least one nucleoside of the sense RNAi oligonucleotide comprises a 2′-OMe modified sugar moiety and at least one nucleoside comprises a 2′-F modified sugar moiety. In certain embodiments, at least 1, 2, 3, 4, 5, 6, 7, 8, 9 or 10 nucleosides comprises a 2′-OMe modified sugar moiety and at least 1, 2, 3, 4, 5, 6, 7, 8, 9 or 10 nucleosides comprises a 2′-F modified sugar moiety. In certain embodiments, the sense RNAi oligonucleotide comprises a sugar motif of fyf or yfy, wherein each “f” represents a 2′-F modified sugar moiety and each “y” represents a 2′-OMe modified sugar moiety. In certain embodiments, the sense RNAi oligonucleotide has a sugar motif of fyfyfyfyfyfyfyfyfyf, wherein each “f” represents a 2′-F modified sugar moiety and each “y” represents a 2′-OMe modified sugar moiety.

2. Certain Nucleobase Motifs

In certain embodiments, oligonucleotides comprise modified and/or unmodified nucleobases arranged along the oligonucleotide or region thereof in a defined pattern or motif. In certain embodiments, each nucleobase is modified. In certain embodiments, none of the nucleobases are modified. In certain embodiments, each purine or each pyrimidine is modified. In certain embodiments, each adenine is modified. In certain embodiments, each guanine is modified. In certain embodiments, each thymine is modified. In certain embodiments, each uracil is modified. In certain embodiments, each cytosine is modified. In certain embodiments, some or all of the cytosine nucleobases in a modified oligonucleotide are 5-methyl cytosines. In certain embodiments, all of the cytosine nucleobases are 5-methyl cytosines and all of the other nucleobases of the modified oligonucleotide are unmodified nucleobases.

In certain embodiments, modified oligonucleotides comprise a block of modified nucleobases. In certain such embodiments, the block is at the 3′-end of the oligonucleotide. In certain embodiments the block is within 3 nucleosides of the 3′-end of the oligonucleotide. In certain embodiments, the block is at the 5′-end of the oligonucleotide. In certain embodiments the block is within 3 nucleosides of the 5′-end of the oligonucleotide.

### Gapmer Oligonucleotides

In certain embodiments, oligonucleotides having a gapmer motif comprise a nucleoside comprising a modified nucleobase. In certain such embodiments, one nucleoside comprising a modified nucleobase is in the central gap of an oligonucleotide having a gapmer motif. In certain such embodiments, the sugar moiety of said nucleoside is a 2′-deoxyribosyl moiety. In certain embodiments, the modified nucleobase is selected from: a 2-thiopyrimidine and a 5-propynepyrimidine.

### Antisense RNAi Oligonucleotides

In certain embodiments, one nucleoside of an antisense RNAi oligonucleotide is a UNA. In certain embodiments, one nucleoside of an antisense RNAi oligonucleotide is a GNA. In certain embodiments, 1-4 nucleosides of an antisense RNAi oligonucleotide is/are DNA. In certain such embodiments, the 1-4 DNA nucleosides are at one or both ends of the antisense RNAi oligonucleotide.

### Sense RNAi Oligonucleotides

In certain embodiments, one nucleoside of a sense RNAi oligonucleotide is a UNA. In certain embodiments, one nucleoside of a sense RNAi oligonucleotide is a GNA. In certain embodiments, 1-4 nucleosides of a sense RNAi oligonucleotide is/are DNA. In certain such embodiments, the 1-4 DNA nucleosides are at one or both ends of the sense RNAi oligonucleotide.

3. Certain Internucleoside Linkage Motifs

In certain embodiments, oligonucleotides comprise modified and/or unmodified internucleoside linkages arranged along the oligonucleotide or region thereof in a defined pattern or motif. In certain embodiments, each internucleoside linking group is a phosphodiester internucleoside linkage (P═O). In certain embodiments, each internucleoside linking group of a modified oligonucleotide is a phosphorothioate internucleoside linkage (P═S). In certain embodiments, each internucleoside linkage of a modified oligonucleotide is independently selected from a phosphorothioate internucleoside linkage and phosphodiester internucleoside linkage. In certain embodiments, each phosphorothioate internucleoside linkage is independently selected from a stereorandom phosphorothioate a (Sp) phosphorothioate, and a (Rp) phosphorothioate.

### Gapmer Oligonucleotides

In certain embodiments, the sugar motif of a modified oligonucleotide is a gapmer and the internucleoside linkages within the gap are all modified. In certain such embodiments, some or all of the internucleoside linkages in the wings are unmodified phosphodiester internucleoside linkages. In certain embodiments, the terminal internucleoside linkages are modified. In certain embodiments, the sugar motif of a modified oligonucleotide is a gapmer, and the internucleoside linkage motif comprises at least one phosphodiester internucleoside linkage in at least one wing, wherein the at least one phosphodiester linkage is not a terminal internucleoside linkage, and the remaining internucleoside linkages are phosphorothioate internucleoside linkages. In certain such embodiments, all of the phosphorothioate linkages are stereorandom. In certain embodiments, all of the phosphorothioate linkages in the wings are (Sp) phosphorothioates, and the gap comprises at least one Sp, Sp, Rp motif. In certain embodiments, populations of modified oligonucleotides are enriched for modified oligonucleotides comprising such internucleoside linkage motifs.

### Antisense RNAi Oligonucleotides

In certain embodiments, at least one linkage of the antisense RNAi oligonucleotide is a modified linkage. In certain embodiments, the 5′-most linkage (i.e., linking the first nucleoside from the 5′-end to the second nucleoside from the 5′-end) is modified. In certain embodiments, the two 5′-most linkages are modified. In certain embodiments, the first one or 2 linkages from the 3′-end are modified. In certain such embodiments, the modified linkage is a phosphorothioate linkage. In certain embodiments, the remaining linkages are all unmodified phosphodiester linkages. In certain embodiments, antisense RNAi oligonucleotides have an internucleoside linkage motif of ssooooooooooooooooooss, wherein each “s” represents a phosphorothioate internucleoside linkage and each “o” represents a phosphate internucleoside linkage. In certain embodiments, at least one linkage of the antisense RNAi oligonucleotide is an inverted linkage.

### Sense RNAi Oligonucleotides

In certain embodiments, at least one linkage of the sense RNAi oligonucleotides is a modified linkage. In certain embodiments, the 5′-most linkage (i.e., linking the first nucleoside from the 5′-end to the second nucleoside from the 5′-end) is modified. In certain embodiments, the two 5′-most linkages are modified. In certain embodiments, the first one or 2 linkages from the 3′-end are modified. In certain such embodiments, the modified linkage is a phosphorothioate linkage. In certain embodiments, the remaining linkages are all unmodified phosphodiester linkages. In certain embodiments, sense RNAi oligonucleotides have an internucleoside linkage motif of ssooooooooooooooooss, wherein each “s” represents a phosphorothioate internucleoside linkage and each “o” represents a phosphate internucleoside linkage. In certain embodiments, at least one linkage of the sense RNAi oligonucleotides is an inverted linkage.

C. Certain Lengths

It is possible to increase or decrease the length of an oligonucleotide without eliminating activity. For example, in Woolf et al. (Proc. Natl. Acad. Sci. USA 89:7305-7309, 1992), a series of oligonucleotides 13-25 nucleobases in length were tested for their ability to induce cleavage of a target RNA in an oocyte injection model. Oligonucleotides 25 nucleobases in length with 8 or 11 mismatch bases near the ends of the oligonucleotides were able to direct specific cleavage of the target RNA, albeit to a lesser extent than the oligonucleotides that contained no mismatches. Similarly, target specific cleavage was achieved using 13 nucleobase oligonucleotides, including those with 1 or 3 mismatches.

In certain embodiments, oligonucleotides (including modified oligonucleotides) can have any of a variety of ranges of lengths. In certain embodiments, oligonucleotides consist of X to Y linked nucleosides, where X represents the fewest number of nucleosides in the range and Y represents the largest number nucleosides in the range. In certain such embodiments, X and Y are each independently selected from 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, and 50; provided that X≤Y. For example, in certain embodiments, oligonucleotides consist of 12 to 13, 12 to 14, 12 to 15, 12 to 16, 12 to 17, 12 to 18, 12 to 19, 12 to 20, 12 to 21, 12 to 22, 12 to 23, 12 to 24, 12 to 25, 12 to 26, 12 to 27, 12 to 28, 12 to 29, 12 to 30, 13 to 14, 13 to 15, 13 to 16, 13 to 17, 13 to 18, 13 to 19, 13 to 20, 13 to 21, 13 to 22, 13 to 23, 13 to 24, 13 to 25, 13 to 26, 13 to 27, 13 to 28, 13 to 29, 13 to 30, 14 to 15, 14 to 16, 14 to 17, 14 to 18, 14 to 19, 14 to 20, 14 to 21, 14 to 22, 14 to 23, 14 to 24, 14 to 25, 14 to 26, 14 to 27, 14 to 28, 14 to 29, 14 to 30, 15 to 16, 15 to 17, 15 to 18, 15 to 19, 15 to 20, 15 to 21, 15 to 22, 15 to 23, 15 to 24, 15 to 25, 15 to 26, 15 to 27, 15 to 28, 15 to 29, 15 to 30, 16 to 17, 16 to 18, 16 to 19, 16 to 20, 16 to 21, 16 to 22, 16 to 23, 16 to 24, 16 to 25, 16 to 26, 16 to 27, 16 to 28, 16 to 29, 16 to 30, 17 to 18, 17 to 19, 17 to 20, 17 to 21, 17 to 22, 17 to 23, 17 to 24, 17 to 25, 17 to 26, 17 to 27, 17 to 28, 17 to 29, 17 to 30, 18 to 19, 18 to 20, 18 to 21, 18 to 22, 18 to 23, 18 to 24, 18 to 25, 18 to 26, 18 to 27, 18 to 28, 18 to 29, 18 to 30, 19 to 20, 19 to 21, 19 to 22, 19 to 23, 19 to 24, 19 to 25, 19 to 26, 19 to 29, 19 to 28, 19 to 29, 19 to 30, 20 to 21, 20 to 22, 20 to 23, 20 to 24, 20 to 25, 20 to 26, 20 to 27, 20 to 28, 20 to 29, 20 to 30, 21 to 22, 21 to 23, 21 to 24, 21 to 25, 21 to 26, 21 to 27, 21 to 28, 21 to 29, 21 to 30, 22 to 23, 22 to 24, 22 to 25, 22 to 26, 22 to 27, 22 to 28, 22 to 29, 22 to 30, 23 to 24, 23 to 25, 23 to 26, 23 to 27, 23 to 28, 23 to 29, 23 to 30, 24 to 25, 24 to 26, 24 to 27, 24 to 28, 24 to 29, 24 to 30, 25 to 26, 25 to 27, 25 to 28, 25 to 29, 25 to 30, 26 to 27, 26 to 28, 26 to 29, 26 to 30, 27 to 28, 27 to 29, 27 to 30, 28 to 29, 28 to 30, or 29 to 30 linked nucleosides.

### Antisense RNAi Oligonucleotides

In certain embodiments, antisense RNAi oligonucleotides consist of 17-30 linked nucleosides. In certain embodiments, antisense RNAi oligonucleotides consist of 17-25 linked nucleosides. In certain embodiments, antisense RNAi oligonucleotides consist of 17-23 linked nucleosides. In certain embodiments, antisense RNAi oligonucleotides consist of 17-21 linked nucleosides. In certain embodiments, antisense RNAi oligonucleotides consist of 18-30 linked nucleosides. In certain embodiments, antisense RNAi oligonucleotides consist of 20-30 linked nucleosides. In certain embodiments, antisense RNAi oligonucleotides consist of 21-30 linked nucleosides. In certain embodiments, antisense RNAi oligonucleotides consist of 23-30 linked nucleosides. In certain embodiments, antisense RNAi oligonucleotides consist of 18-25 linked nucleosides. In certain embodiments, antisense RNAi oligonucleotides consist of 20-22 linked nucleosides. In certain embodiments, antisense RNAi oligonucleotides consist of 21-23 linked nucleosides. In certain embodiments, antisense RNAi oligonucleotides consist of 23-24 linked nucleosides. In certain embodiments, antisense RNAi oligonucleotides consist of 20 linked nucleosides. In certain embodiments, antisense RNAi oligonucleotides consist of 21 linked nucleosides. In certain embodiments, antisense RNAi oligonucleotides consist of 22 linked nucleosides. In certain embodiments, antisense RNAi oligonucleotides consist of 23 linked nucleosides.

### Sense RNAi Oligonucleotides

In certain embodiments, sense RNAi oligonucleotides consist of 17-30 linked nucleosides. In certain embodiments, sense RNAi oligonucleotides consist of 17-25 linked nucleosides. In certain embodiments, sense RNAi oligonucleotides consist of 17-23 linked nucleosides. In certain embodiments, sense RNAi oligonucleotides consist of 17-21 linked nucleosides. In certain embodiments, sense RNAi oligonucleotides consist of 18-30 linked nucleosides. In certain embodiments, sense RNAi oligonucleotides consist of 20-30 linked nucleosides. In certain embodiments, sense RNAi oligonucleotides consist of 21-30 linked nucleosides. In certain embodiments, sense RNAi oligonucleotides consist of 23-30 linked nucleosides. In certain embodiments, sense RNAi oligonucleotides consist of 18-25 linked nucleosides. In certain embodiments, sense RNAi oligonucleotides consist of 20-22 linked nucleosides. In certain embodiments, sense RNAi oligonucleotides consist of 21-23 linked nucleosides. In certain embodiments, sense RNAi oligonucleotides consist of 23-24 linked nucleosides. In certain embodiments, sense RNAi oligonucleotides consist of 20 linked nucleosides. In certain embodiments, sense RNAi oligonucleotides consist of 21 linked nucleosides. In certain embodiments, sense RNAi oligonucleotides consist of 22 linked nucleosides. In certain embodiments, sense RNAi oligonucleotides consist of 23 linked nucleosides.

D. Certain Modified Oligonucleotides

In certain embodiments, the above modifications (sugar, nucleobase, internucleoside linkage) are incorporated into a modified oligonucleotide. In certain embodiments, modified oligonucleotides are characterized by their modification motifs and overall lengths. In certain embodiments, such parameters are each independent of one another. Thus, unless otherwise indicated, each internucleoside linkage of an oligonucleotide having a gapmer sugar motif may be modified or unmodified and may or may not follow the gapmer modification pattern of the sugar modifications. For example, the internucleoside linkages within the wing regions of a sugar gapmer may be the same or different from one another and may be the same or different from the internucleoside linkages of the gap region of the sugar motif. Likewise, such sugar gapmer oligonucleotides may comprise one or more modified nucleobase independent of the gapmer pattern of the sugar modifications. Unless otherwise indicated, all modifications are independent of nucleobase sequence.

E. Certain Populations of Modified Oligonucleotides

Populations of modified oligonucleotides in which all of the modified oligonucleotides of the population have the same molecular formula can be stereorandom populations or chirally enriched populations. All of the chiral centers of all of the modified oligonucleotides are stereorandom in a stereorandom population. In a chirally enriched population, at least one particular chiral center is not stereorandom in the modified oligonucleotides of the population. In certain embodiments, the modified oligonucleotides of a chirally enriched population are enriched for β-D ribosyl sugar moieties, and all of the phosphorothioate internucleoside linkages are stereorandom. In certain embodiments, the modified oligonucleotides of a chirally enriched population are enriched for both β-D ribosyl sugar moieties and at least one, particular phosphorothioate internucleoside linkage in a particular stereochemical configuration.

F. Nucleobase Sequence

In certain embodiments, oligonucleotides (unmodified or modified oligonucleotides) are further described by their nucleobase sequence. In certain embodiments oligonucleotides have a nucleobase sequence that is complementary to a second oligonucleotide or an identified reference nucleic acid, such as a target nucleic acid. In certain such embodiments, a region of an oligonucleotide has a nucleobase sequence that is complementary to a second oligonucleotide or an identified reference nucleic acid, such as a target nucleic acid. In certain embodiments, the nucleobase sequence of a region or entire length of an oligonucleotide is at least 50%, at least 60%, at least 70%, at least 80%, at least 85%, at least 90%, at least 95%, or 100% complementary to the second oligonucleotide or nucleic acid, such as a target nucleic acid.

II. Certain Oligomeric Compounds

In certain embodiments, provided herein are oligomeric compounds, which consist of an oligonucleotide (modified or unmodified) and optionally one or more conjugate groups and/or terminal groups. Conjugate groups consist of one or more conjugate moiety and a conjugate linker which links the conjugate moiety to the oligonucleotide. Conjugate groups may be attached to either or both ends of an oligonucleotide and/or at any internal position. In certain embodiments, conjugate groups are attached to the 2′-position of a nucleoside of a modified oligonucleotide. In certain embodiments, conjugate groups that are attached to either or both ends of an oligonucleotide are terminal groups. In certain such embodiments, conjugate groups or terminal groups are attached at the 3′ and/or 5′-end of oligonucleotides. In certain such embodiments, conjugate groups (or terminal groups) are attached at the 3′-end of oligonucleotides. In certain embodiments, conjugate groups are attached near the 3′-end of oligonucleotides. In certain embodiments, conjugate groups (or terminal groups) are attached at the 5′-end of oligonucleotides. In certain embodiments, conjugate groups are attached near the 5′-end of oligonucleotides.

Examples of terminal groups include but are not limited to conjugate groups, capping groups, phosphate moieties, protecting groups, modified or unmodified nucleosides, and two or more nucleosides that are independently modified or unmodified.

A. Certain RNAi Compounds

RNAi compounds comprise an antisense RNAi oligonucleotide and optionally a sense RNAi oligonucleotide. RNAi compounds may also comprise terminal groups and/or conjugate groups which may be attached to the antisense RNAi oligonucleotide or the sense RNAi oligonucleotide (when present).

Duplexes

RNAi compounds comprising an antisense RNAi oligonucleotide and a sense RNAi oligonucleotide form a duplex, because the sense RNAi oligonucleotide comprises an antisense-hybridizing region that is complementary to the antisense RNAi oligonucleotide. In certain embodiments, each nucleobase of the antisense RNAi oligonucleotide and the sense RNAi oligonucleotide are complementary to one another. In certain embodiments, the two RNAi oligonucleotides have at least one mismatch relative to one another.

In certain embodiments, the antisense hybridizing region constitutes the entire length of the sense RNAi oligonucleotide and the antisense RNAi oligonucleotide. In certain embodiments, one or both of the antisense RNAi oligonucleotide and the sense RNAi oligonucleotide comprise additional nucleosides at one or both ends that do not hybridize (overhanging nucleosides). In certain embodiments, overhanging nucleosides are DNA. In certain embodiments, overhanging nucleosides are linked to each other (where there is more than one) and to the first non-overhanging nucleoside with phosphorothioate linkages.

B. Certain Conjugate Groups

In certain embodiments, oligonucleotides are covalently attached to one or more conjugate groups. In certain embodiments, conjugate groups modify one or more properties of the attached oligonucleotide, including but not limited to pharmacodynamics, pharmacokinetics, stability, binding, absorption, tissue distribution, cellular distribution, cellular uptake, charge and clearance. In certain embodiments, conjugate groups impart a new property on the attached oligonucleotide, e.g., fluorophores or reporter groups that enable detection of the oligonucleotide. Certain conjugate groups and conjugate moieties have been described previously, for example: cholesterol moiety (Letsinger et al., Proc. Natl. Acad. Sci. USA, 1989, 86, 6553-6556), cholic acid (Manoharan et al., Bioorg. Med. Chem. Lett., 1994, 4, 1053-1060), a thioether, e.g., hexyl-S-tritylthiol (Manoharan et al., Ann. N. Y. Acad. Sci., 1992, 660, 306-309; Manoharan et al., Bioorg. Med. Chem. Lett., 1993, 3, 2765-2770), a thiocholesterol (Oberhauser et al., Nucl. Acids Res., 1992, 20, 533-538), an aliphatic chain, e.g., do-decan-diol or undecyl residues (Saison-Behmoaras et al., EMBO J., 1991, 10, 1111-1118; Kabanov et al., FEBS Lett., 1990, 259, 327-330; Svinarchuk et al., Biochimie, 1993, 75, 49-54), a phospholipid, e.g., di-hexadecyl-rac-glycerol or triethyl-ammonium 1,2-di-O-hexadecyl-rac-glycero-3-H-phosphonate (Manoharan et al., Tetrahedron Lett., 1995, 36, 3651-3654; Shea et al., Nucl. Acids Res., 1990, 18, 3777-3783), a polyamine or a polyethylene glycol chain (Manoharan et al., Nucleosides & Nucleotides, 1995, 14, 969-973), or adamantane acetic acid a palmityl moiety (Mishra et al., Biochim. Biophys. Acta, 1995, 1264, 229-237), an octadecylamine or hexylamino-carbonyl-oxycholesterol moiety (Crooke et al., J Pharmacol. Exp. Ther., 1996, 277, 923-937), a tocopherol group (Nishina et al., Molecular Therapy Nucleic Acids, 2015, 4, e220; and Nishina et al., Molecular Therapy, 2008, 16, 734-740), or a GalNAc cluster (e.g., WO2014/179620).

1. Conjugate Moieties

Conjugate moieties include, without limitation, intercalators, reporter molecules, polyamines, polyamides, peptides, carbohydrates, vitamin moieties, polyethylene glycols, thioethers, polyethers, cholesterols, thiocholesterols, cholic acid moieties, folate, lipids, phospholipids, biotin, phenazine, phenanthridine, anthraquinone, adamantane, acridine, fluoresceins, rhodamines, coumarins, fluorophores, and dyes.

In certain embodiments, a conjugate moiety comprises an active drug substance, for example, aspirin, warfarin, phenylbutazone, ibuprofen, suprofen, fen-bufen, ketoprofen, (S)-(+)-pranoprofen, carprofen, dansylsarcosine, 2,3,5-triiodobenzoic acid, fingolimod, flufenamic acid, folinic acid, a benzothiadiazide, chlorothiazide, a diazepine, indo-methicin, a barbiturate, a cephalosporin, a sulfa drug, an antidiabetic, an antibacterial or an antibiotic.

2. Conjugate Linkers

Conjugate moieties are attached to oligonucleotides through conjugate linkers. In certain oligomeric compounds, the conjugate linker is a single chemical bond (i.e., the conjugate moiety is attached directly to an oligonucleotide through a single bond). In certain embodiments, the conjugate linker comprises a chain structure, such as a hydrocarbyl chain, or an oligomer of repeating units such as ethylene glycol, nucleosides, or amino acid units.

In certain embodiments, a conjugate linker comprises one or more groups selected from alkyl, amino, oxo, amide, disulfide, polyethylene glycol, ether, thioether, and hydroxylamino. In certain such embodiments, the conjugate linker comprises groups selected from alkyl, amino, oxo, amide and ether groups. In certain embodiments, the conjugate linker comprises groups selected from alkyl and amide groups. In certain embodiments, the conjugate linker comprises groups selected from alkyl and ether groups. In certain embodiments, the conjugate linker comprises at least one phosphorus moiety. In certain embodiments, the conjugate linker comprises at least one phosphate group. In certain embodiments, the conjugate linker includes at least one neutral linking group.

In certain embodiments, conjugate linkers, including the conjugate linkers described above, are bifunctional linking moieties, e.g., those known in the art to be useful for attaching conjugate groups to parent compounds, such as the oligonucleotides provided herein. In general, a bifunctional linking moiety comprises at least two functional groups. One of the functional groups is selected to bind to a particular site on a parent compound and the other is selected to bind to a conjugate group. Examples of functional groups used in a bifunctional linking moiety include but are not limited to electrophiles for reacting with nucleophilic groups and nucleophiles for reacting with electrophilic groups. In certain embodiments, bifunctional linking moieties comprise one or more groups selected from amino, hydroxyl, carboxylic acid, thiol, alkyl, alkenyl, and alkynyl.

In certain embodiments, a conjugate linker comprises pyrrolidine.

Examples of conjugate linkers include but are not limited to pyrrolidine, 8-amino-3,6-dioxaoctanoic acid (ADO), succinimidyl 4-(N-maleimidomethyl) cyclohexane-1-carboxylate (SMCC) and 6-aminohexanoic acid (AHEX or AHA). Other conjugate linkers include but are not limited to substituted or unsubstituted C₁-C₁₀ alkyl, substituted or unsubstituted C₂-C₁₀ alkenyl or substituted or unsubstituted C₂-C₁₀ alkynyl, wherein a nonlimiting list of preferred substituent groups includes hydroxyl, amino, alkoxy, carboxy, benzyl, phenyl, nitro, thiol, thioalkoxy, halogen, alkyl, aryl, alkenyl and alkynyl.

In certain embodiments, conjugate linkers comprise 1-10 linker-nucleosides. In certain embodiments, conjugate linkers comprise 2-5 linker-nucleosides. In certain embodiments, conjugate linkers comprise exactly 3 linker-nucleosides. In certain embodiments, conjugate linkers comprise the TCA motif. In certain embodiments, such linker-nucleosides are modified nucleosides. In certain embodiments such linker-nucleosides comprise a modified sugar moiety. In certain embodiments, linker-nucleosides are unmodified. In certain embodiments, linker-nucleosides comprise an optionally protected heterocyclic base selected from a purine, substituted purine, pyrimidine or substituted pyrimidine. In certain embodiments, a cleavable moiety is a nucleoside selected from uracil, thymine, cytosine, 4-N-benzoylcytosine, 5-methyl cytosine, 4-N-benzoyl-5-methyl cytosine, adenine, 6-N-benzoyladenine, guanine and 2-N-isobutyrylguanine. It is typically desirable for linker-nucleosides to be cleaved from the oligomeric compound after it reaches a target tissue. Accordingly, linker-nucleosides are typically linked to one another and to the remainder of the oligomeric compound through cleavable bonds. In certain embodiments, such cleavable bonds are phosphodiester bonds.

Herein, linker-nucleosides are not considered to be part of the oligonucleotide. Accordingly, in embodiments in which an oligomeric compound comprises an oligonucleotide consisting of a specified number or range of linked nucleosides and/or a specified percent complementarity to a reference nucleic acid and the oligomeric compound also comprises a conjugate group comprising a conjugate linker comprising linker-nucleosides, those linker-nucleosides are not counted toward the length of the oligonucleotide and are not used in determining the percent complementarity of the oligonucleotide for the reference nucleic acid. For example, an oligomeric compound may comprise (1) a modified oligonucleotide consisting of 8-30 nucleosides and (2) a conjugate group comprising 1-10 linker-nucleosides that are contiguous with the nucleosides of the modified oligonucleotide. The total number of contiguous linked nucleosides in such an oligomeric compound is more than 30. Alternatively, an oligomeric compound may comprise a modified oligonucleotide consisting of 8-30 nucleosides and no conjugate group. The total number of contiguous linked nucleosides in such an oligomeric compound is no more than 30. Unless otherwise indicated conjugate linkers comprise no more than 10 linker-nucleosides. In certain embodiments, conjugate linkers comprise no more than 5 linker-nucleosides. In certain embodiments, conjugate linkers comprise no more than 3 linker-nucleosides. In certain embodiments, conjugate linkers comprise no more than 2 linker-nucleosides. In certain embodiments, conjugate linkers comprise no more than 1 linker-nucleoside.

In certain embodiments, it is desirable for a conjugate group to be cleaved from the oligonucleotide. For example, in certain circumstances oligomeric compounds comprising a particular conjugate moiety are better taken up by a particular cell type, but once the oligomeric compound has been taken up, it is desirable that the conjugate group be cleaved to release the unconjugated or parent oligonucleotide. Thus, certain conjugate linkers may comprise one or more cleavable moieties. In certain embodiments, a cleavable moiety is a cleavable bond. In certain embodiments, a cleavable moiety is a group of atoms comprising at least one cleavable bond. In certain embodiments, a cleavable moiety comprises a group of atoms having one, two, three, four, or more than four cleavable bonds. In certain embodiments, a cleavable moiety is selectively cleaved inside a cell or subcellular compartment, such as a lysosome. In certain embodiments, a cleavable moiety is selectively cleaved by endogenous enzymes, such as nucleases.

In certain embodiments, a cleavable bond is selected from among: an amide, an ester, an ether, one or both esters of a phosphodiester, a phosphate ester, a carbamate, or a disulfide. In certain embodiments, a cleavable bond is one or both of the esters of a phosphodiester. In certain embodiments, a cleavable moiety comprises a phosphate or phosphodiester. In certain embodiments, the cleavable moiety is a phosphate linkage between an oligonucleotide and a conjugate moiety or conjugate group.

In certain embodiments, a cleavable moiety comprises or consists of one or more linker-nucleosides. In certain such embodiments, the one or more linker-nucleosides are linked to one another and/or to the remainder of the oligomeric compound through cleavable bonds. In certain embodiments, such cleavable bonds are unmodified phosphodiester bonds. In certain embodiments, a cleavable moiety is 2′-deoxynucleoside that is attached to either the 3′ or 5′-terminal nucleoside of an oligonucleotide by a phosphate internucleoside linkage and covalently attached to the remainder of the conjugate linker or conjugate moiety by a phosphate or phosphorothioate linkage. In certain such embodiments, the cleavable moiety is 2′-deoxyadenosine.

C. Certain Terminal Groups

In certain embodiments, oligomeric compounds comprise one or more terminal groups. In certain such embodiments, oligomeric compounds comprise a stabilized 5′-phophate. Stabilized 5′-phosphates include, but are not limited to 5′-phosphanates, including, but not limited to 5′-vinylphosphonates. In certain embodiments, terminal groups comprise one or more abasic nucleosides and/or inverted nucleosides. In certain embodiments, terminal groups comprise one or more 2′-linked nucleosides. In certain such embodiments, the 2′-linked nucleoside is an abasic nucleoside.

In certain embodiments, oligomeric compounds comprise one or more terminal groups. In certain such embodiments, modified oligonucleotides comprise a phosphorus-containing group at the 5′-end of the modified oligonucleotide. In certain embodiments, the phosphorus-containing group is at the 5′-end of the antisense RNAi oligonucleotide and/or the sense RNAi oligonucleotide. In certain embodiments, the terminal group is a phosphate stabilized phosphate group. The 5′-end phosphorus-containing group can be 5′-end phosphate (5′-P), 5′-end phosphorothioate (5′-PS), 5′-end phosphorodithioate (5′-PS₂), 5′-end vinylphosphonate (5′-VP), 5′-end methylphosphonate (MePhos) or 5′-deoxy-5′-C-malonyl. When the 5′-end phosphorus-containing group is 5′-end vinylphosphonate, the 5′VP can be either 5′-E-VP isomer (i.e., trans-vinylphosphonate), 5′-Z-VP isomer (i.e., cis-vinylphosphonate), or mixtures thereof. Although such phosphate group can be attached to any modified oligonucleotide, it has particularly been shown that attachment of such a group to an antisense RNAi oligonucleotide improves activity of certain RNAi agents. See, e.g., Prakash et al., Nucleic Acids Res., 43(6):2993-3011, 2015; Elkayam, et al., Nucleic Acids Res., 45(6):3528-3536, 2017; Parmar, et al. ChemBioChem, 17(11)985-989; 2016; Harastzi, et al., Nucleic Acids Res., 45(13):7581-7592, 2017. In certain embodiments, the phosphate stabilizing group is 5′-cyclopropyl phosphonate. See e.g., WO/2018/027106.

In certain embodiments, terminal groups comprise one or more abasic nucleosides and/or inverted nucleosides. In certain embodiments, terminal groups comprise one or more 2′-linked nucleosides. In certain such embodiments, the 2′-linked nucleoside is an abasic nucleoside.

D. Certain Specific RNAi Motifs

RNAi agents can be described by motif or by specific features.

In certain embodiments, the RNAi agents described herein comprise:

(a) a sense RNAi oligonucleotide having:
(i) a length of 21 nucleotides;
(ii) a conjugate attached to the 3′-end; and
(iii) 2′-F modifications at positions 1, 3, 5, 7, 9 to 11, 13, 17, 19, and 21, and 2′-OMe modifications at positions 2, 4, 6, 8, 12, 14 to 16, 18, and 20 (counting from the 5′ end);

and

(b) an antisense RNAi oligonucleotide having:
(i) a length of 23 nucleotides;
(ii) 2′-OMe modifications at positions 1, 3, 5, 9, 11 to 13, 15, 17, 19, 21, and 23, and 2′F modifications at positions 2, 4, 6 to 8, 10, 14, 16, 18, 20, and 22 (counting from the 5′ end); and
(iii) phosphorothioate internucleoside linkages between nucleoside positions 21 and 22, and between nucleoside positions 22 and 23 (counting from the 5′ end);
wherein the two nucleotides at the 3′end of the antisense RNAi oligonucleotide are overhanging nucleosides, and the end of the RNAi agent duplex constituting the 5′-end of the antisense RNAi oligonucleotide and the 3′-end of the sense RNAi oligonucleotide is blunt (i.e., neither oligonucleotide has overhang nucleoside at that end and instead the hybridizing region of the sense RNAi oligonucleotide includes the 3′-most nucleoside of the sense RNAi oligonucleotide and that nucleoside hybridizes with the 5′-most nucleoside of the antisense oligonucleotide).

In certain embodiments, the RNAi agents described herein comprise:

(a) a sense RNAi oligonucleotide having:
(i) a length of 21 nucleotides;
(ii) a conjugate attached to the 3′-end;
(iii) 2′-F modifications at positions 1, 3, 5, 7, 9 to 11, 13, 17, 19, and 21, and 2′-OMe modifications at positions 2, 4, 6, 8, 12, 14, 16, 18, and 20 (counting from the 5′ end); and
(iv) phosphorothioate internucleoside linkages between nucleoside positions 1 and 2, and between nucleoside positions 2 and 3 (counting from the 5′ end);

and

(b) an antisense RNAi oligonucleotide having:
(i) a length of 23 nucleotides;
(ii) 2′-OMe modifications at positions 1, 3, 5, 7, 9, 11 to 13, 15, 17, 19, and 21 to 23, and 2′F modifications at positions 2, 4, 6, 8, 10, 14, 16, 18, and 20 (counting from the 5′ end); and
(iii) phosphorothioate internucleoside linkages between nucleoside positions 1 and 2, between nucleoside positions 2 and 3, between nucleoside positions 21 and 22, and between nucleoside positions 22 and 23 (counting from the 5′ end);
wherein the RNAi duplex includes a two nucleotide overhang at the 3′end of the antisense RNAi oligonucleotide, and a blunt end at the 5′-end of the antisense RNAi oligonucleotide.

In certain embodiments, the RNAi agents described herein comprise:

(a) a sense RNAi oligonucleotide having:
(i) a length of 21 nucleotides;
(ii) a conjugate attached to the 3′-end;
(iii) 2′-OMe modifications at positions 1 to 6, 8, 10, and 12 to 21, and 2′-F modifications at positions 7 and 9, and a deoxynucleotide at position 11 (counting from the 5′ end); and
(iv) phosphorothioate internucleoside linkages between nucleoside positions 1 and 2, and between nucleoside positions 2 and 3 (counting from the 5′ end);

and

(b) an antisense RNAi oligonucleotide having:
(i) a length of 23 nucleotides;
(ii) 2′-OMe modifications at positions 1, 3, 7, 9, 11, 13, 15, 17, and 19 to 23, and 2′F modifications at positions 2, 4 to 6, 8, 10, 12, 14, 16, and 18 (counting from the 5′ end); and
(iii) phosphorothioate internucleoside linkages between nucleoside positions 1 and 2, between nucleoside positions 2 and 3, between nucleoside positions 21 and 22, and between nucleoside positions 22 and 23 (counting from the 5′ end);
wherein the RNAi duplex has a two nucleotide overhang at the 3′end of the antisense RNAi oligonucleotide, and a blunt end at the 5′-end of the antisense RNAi oligonucleotide.

In certain embodiments, the RNAi agents described herein comprise:

(a) a sense RNAi oligonucleotide having:
(i) a length of 21 nucleotides;
(ii) a conjugate attached to the 3′-end;
(iii) 2′-OMe modifications at positions 1 to 6, 8, and 12 to 21, and 2′-F modifications at positions 7, and 9 to 11; and
(iv) phosphorothioate internucleoside linkages between nucleoside positions 1 and 2, and between nucleoside positions 2 and 3 (counting from the 5′ end);

and

(b) an antisense RNAi oligonucleotide having:
(i) a length of 23 nucleotides;
(ii) 2′-OMe modifications at positions 1, 3 to 5, 7, 8, 10 to 13, 15, and 17 to 23, and 2′F modifications at positions 2, 6, 9, 14, and 16 (counting from the 5′ end); and
(iii) phosphorothioate internucleoside linkages between nucleoside positions 1 and 2, between nucleoside positions 2 and 3, between nucleoside positions 21 and 22, and between nucleoside positions 22 and 23 (counting from the 5′ end);
wherein the RNAi duplex has a two nucleotide overhang at the 3′end of the antisense RNAi oligonucleotide, and a blunt end at the 5′-end of the antisense RNAi oligonucleotide.

In certain embodiments, the RNAi agents described herein comprise:

(a) a sense RNAi oligonucleotide having:
(i) a length of 21 nucleotides;
(ii) a conjugate attached to the 3′-end;
(iii) 2′-OMe modifications at positions 1 to 6, 8, and 12 to 21, and 2′-F modifications at positions 7, and 9 to 11; and
(iv) phosphorothioate internucleoside linkages between nucleoside positions 1 and 2, and between nucleoside positions 2 and 3 (counting from the 5′ end);

and

(b) an antisense RNAi oligonucleotide having:
(i) a length of 23 nucleotides;
(ii) 2′-OMe modifications at positions 1, 3 to 5, 7, 10 to 13, 15, and 17 to 23, and 2′F modifications at positions 2, 6, 8, 9, 14, and 16 (counting from the 5′ end); and
(iii) phosphorothioate internucleoside linkages between nucleoside positions 1 and 2, between nucleoside positions 2 and 3, between nucleoside positions 21 and 22, and between nucleoside positions 22 and 23 (counting from the 5′ end);
wherein the RNAi duplex has a two nucleotide overhang at the 3′end of the antisense RNAi oligonucleotide, and a blunt end at the 5′-end of the antisense RNAi oligonucleotide.

In certain embodiments, the RNAi agents described herein comprise:

(a) a sense RNAi oligonucleotide having:
(i) a length of 19 nucleotides;
(ii) a conjugate attached to the 3′-end;
(iii) 2′-OMe modifications at positions 1 to 4, 6, and 10 to 19, and 2′-F modifications at positions 5, and 7 to 9; and
(iv) phosphorothioate internucleoside linkages between nucleoside positions 1 and 2, and between nucleoside positions 2 and 3 (counting from the 5′ end);

and

(b) an antisense RNAi oligonucleotide having:
(i) a length of 21 nucleotides;
(ii) 2′-OMe modifications at positions 1, 3 to 5, 7, 10 to 13, 15, and 17 to 21, and 2′F modifications at positions 2, 6, 8, 9, 14, and 16 (counting from the 5′ end); and
(iii) phosphorothioate internucleoside linkages between nucleoside positions 1 and 2, between nucleoside positions 2 and 3, between nucleoside positions 19 and 20, and between nucleoside positions 20 and 21 (counting from the 5′ end);
wherein the RNAi duplex has a two nucleotide overhang at the 3′end of the antisense RNAi oligonucleotide, and a blunt end at the 5′-end of the antisense RNAi oligonucleotide.

In certain embodiments, the RNAi agents described herein comprise:

(a) a sense RNAi oligonucleotide having:
(i) a length of 21 nucleotides;
(ii) a conjugate attached at position 6 (counting from the 5′ end);
(iii) 2′-F modifications at positions 7 and 9 to 11, and 2′-OMe modifications at positions 1 to 5, 8, and 12 to 21 (counting from the 5′ end); and
(iv) phosphorothioate internucleoside linkages between nucleoside positions 1 and 2, between nucleoside positions 2 and 3, between nucleoside positions 19 and 20, and between nucleoside positions 20 and 21 (counting from the 5′ end);

and

(b) an antisense RNAi oligonucleotide having:
(i) a length of 23 nucleotides;
(ii) 2′-OMe modifications at positions 1, 3 to 5, 7, 10 to 13, 15, and 17 to 23, and 2′F modifications at positions 2, 6, 8, 9, 14, and 16 (counting from the 5′ end);
(iii) phosphorothioate internucleoside linkages between nucleoside positions 1 and 2, between nucleoside positions 2 and 3, between nucleoside positions 21 and 22, and between nucleoside positions 22 and 23 (counting from the 5′ end); and
(iv) a stabilized phosphate group attached to the 5′ position of the 5′-most nucleoside;
wherein the RNAi duplex includes a two nucleotide overhang at the 3′end of the antisense RNAi oligonucleotide, and a blunt end at the 5′-end of the antisense RNAi oligonucleotide.

In certain embodiments, the RNAi agents described herein comprise:

(a) a sense RNAi oligonucleotide having:
(i) a length of 21 nucleotides;
(ii) a conjugate attached to the 3′-end;
(iii) 2′-F modifications at positions 7 and 9 to 11, and 2′-OMe modifications at positions 1 to 6, 8, and 12 to 21 (counting from the 5′ end);
(iv) phosphorothioate internucleoside linkages between nucleoside positions 1 and 2 and between nucleoside positions 2 and 3 (counting from the 5′ end);

and

(b) an antisense RNAi oligonucleotide having:
(i) a length of 23 nucleotides;
(ii) 2′-OMe modifications at positions 1, 3 to 5, 7 to 13, 15, and 17 to 23 an (S)-GNA modification at position 6, and 2′F modifications at positions 2, 14, and 16 (counting from the 5′ end); and
(iii) phosphorothioate internucleoside linkages between nucleoside positions 1 and 2, between nucleoside positions 2 and 3, between nucleoside positions 21 and 22, and between nucleoside positions 22 and 23 (counting from the 5′ end);
wherein the RNAi duplex includes a two nucleotide overhang at the 3′end of the antisense RNAi oligonucleotide, and a blunt end at the 5′-end of the antisense RNAi oligonucleotide.

In certain embodiments, the RNAi agents described herein comprise:

(a) a sense RNAi oligonucleotide having:
(i) a length of 21 nucleotides;
(ii) a conjugate attached to the 3′-end;
(iii) 2′-F modifications at positions 7 and 9 to 11, and 2′-OMe modifications at positions 1 to 6, 8, and 12 to 21 (counting from the 5′ end);
(iv) phosphorothioate internucleoside linkages between nucleoside positions 1 and 2 and between nucleoside positions 2 and 3 (counting from the 5′ end);

and

(b) an antisense RNAi oligonucleotide having:
(i) a length of 23 nucleotides;
(ii) 2′-OMe modifications at positions 1, 3 to 6, 8 to 13, 15, and 17 to 23 an (S)-GNA modification at position 7, and 2′F modifications at positions 2, 14, and 16 (counting from the 5′ end); and
(iii) phosphorothioate internucleoside linkages between nucleoside positions 1 and 2, between nucleoside positions 2 and 3, between nucleoside positions 21 and 22, and between nucleoside positions 22 and 23 (counting from the 5′ end);
wherein the RNAi duplex includes a two nucleotide overhang at the 3′end of the antisense RNAi oligonucleotide, and a blunt end at the 5′-end of the antisense RNAi oligonucleotide.

In certain embodiments, the RNAi agents described herein comprise:

(a) a sense RNAi oligonucleotide having:
(i) a length of 21 nucleotides;
(ii) a conjugate attached at position 6 (counting from the 5′ end); and
(iii) 2′-F modifications at positions 7 and 9 to 11, and 2′-OMe modifications at positions 1 to 5, 8, and 12 to 21 (counting from the 5′ end);
(iv) phosphorothioate internucleoside linkages between nucleoside positions 1 and 2, between nucleoside positions 2 and 3, between nucleoside positions 19 and 20, and between nucleoside positions 20 and 21 (counting from the 5′ end);

and

(b) an antisense RNAi oligonucleotide having:
(i) a length of 23 nucleotides;
(ii) 2′-OMe modifications at positions 1, 3 to 5, 7 to 13, 15, and 17 to 23 an (S)-GNA modification at position 6, and 2′F modifications at positions 2, 14, and 16 (counting from the 5′ end);
(iii) phosphorothioate internucleoside linkages between nucleoside positions 1 and 2, between nucleoside positions 2 and 3, between nucleoside positions 21 and 22, and between nucleoside positions 22 and 23 (counting from the 5′ end); and
(iv) a stabilized phosphate group attached to the 5′ position of the 5′-most nucleoside;
wherein the RNAi duplex includes a two nucleotide overhang at the 3′end of the antisense RNAi oligonucleotide, and a blunt end at the 5′-end of the antisense RNAi oligonucleotide.

In certain embodiments, the RNAi agents described herein comprise:

(a) a sense RNAi oligonucleotide having:
(i) a length of 21 nucleotides;
(ii) a conjugate attached at position 6 (counting from the 5′ end);
(iii) 2′-F modifications at positions 7 and 9 to 11, and 2′-OMe modifications at positions 1 to 5, 8, and 12 to 21 (counting from the 5′ end); and
(iv) phosphorothioate internucleoside linkages between nucleoside positions 1 and 2, between nucleoside positions 2 and 3, between nucleoside positions 19 and 20, and between nucleoside positions 20 and 21 (counting from the 5′ end);

and

(b) an antisense RNAi oligonucleotide having:
(i) a length of 23 nucleotides;
(ii) 2′-OMe modifications at positions 1, 3 to 6, 8 to 13, 15, and 17 to 23 an (S)-GNA modification at position 7, and 2′F modifications at positions 2, 14, and 16 (counting from the 5′ end);
(iii) phosphorothioate internucleoside linkages between nucleoside positions 1 and 2, between nucleoside positions 2 and 3, between nucleoside positions 21 and 22, and between nucleoside positions 22 and 23 (counting from the 5′ end); and
(iv) a stabilized phosphate group attached to the 5′ position of the 5′-most nucleoside;
wherein the two nucleotides at the 3′end of the antisense RNAi oligonucleotide are overhanging nucleosides, and the end of the RNAi agent duplex constituting the 5′-end of the antisense RNAi oligonucleotide and the 3′-end of the sense RNAi oligonucleotide is blunt (i.e., neither oligonucleotide has overhang nucleoside at that end and instead the hybridizing region of the sense RNAi oligonucleotide includes the 3′-most nucleoside of the sense RNAi oligonucleotide and that nucleoside hybridizes with the 5′-most nucleoside of the antisense oligonucleotide).

In certain embodiments, the RNAi agents described herein comprise:

(a) a sense RNAi oligonucleotide having:
(i) a length of 21 nucleotides;
(ii) a conjugate attached to the 5′-end;
(iii) 2′-OMe modifications at positions 1 to 8, and 12 to 21, and 2′-F modifications at positions 9 to 11; and
(iv) inverted abasic sugar moieties attached to both the 5′-most and 3′-most nucleosides;

and

(b) an antisense RNAi oligonucleotide having:
(i) a length of 21 nucleotides;
(ii) 2′-OMe modifications at positions 1, 3, 5, 7, 9, 11, 13, 15, 17, 19, and 21, and 2′F modifications at positions 2, 4, 6, 8, 10, 12, 14, 16, 18, and 20 (counting from the 5′ end); and
(iii) phosphorothioate internucleoside linkages between nucleoside positions 1 and 2, between nucleoside positions 2 and 3, between nucleoside positions 3 and 4, and between nucleoside positions 20 and 21 (counting from the 5′ end).

In certain embodiments, the RNAi agents described herein comprise:

(a) a sense RNAi oligonucleotide having:
(i) a length of 21 nucleotides;
(ii) a conjugate attached to the 5′-end;
(iii) 2′-OMe modifications at positions 1 to 8, and 12 to 21, and 2′-F modifications at positions 9 to 11;
(iv) a phosphorothioate internucleoside linkage between nucleoside positions 1 and 2 (counting from the 5′ end); and
(v) an inverted abasic sugar moiety attached to the 3′-most nucleoside;

and

(b) an antisense RNAi oligonucleotide having:
(i) a length of 21 nucleotides;
(ii) 2′-OMe modifications at positions 1, 3, 5, 7, 9, 11, 13, 15, 17, 19, and 21, and 2′F modifications at positions 2, 4, 6, 8, 10, 12, 14, 16, 18, and 20 (counting from the 5′ end); and
(iii) phosphorothioate internucleoside linkages between nucleoside positions 1 and 2, between nucleoside positions 2 and 3, between nucleoside positions 3 and 4, and between nucleoside positions 20 and 21 (counting from the 5′ end).

In certain embodiments, the RNAi agents described herein comprise:

(a) a sense RNAi oligonucleotide having:
(i) a length of 19 nucleotides;
(ii) a conjugate attached to the 5′-end;
(iii) 2′-OMe modifications at positions 2, 4, 6, 8, 10, 12, 14, 16, 18, and 20, and 2′-F modifications at positions 1, 3, 5, 7, 9, 11, 13, 15, 17, 19, and 21; and
(iv) phosphorothioate internucleoside linkages between nucleoside positions 17 and 18, and between nucleoside positions 18 and 19 (counting from the 5′ end);

and

(b) an antisense RNAi oligonucleotide having:
(i) a length of 19 nucleotides;
(ii) 2′-OMe modifications at positions 1, 3, 5, 7, 9, 11, 13, 15, 17, 19, and 21, and 2′F modifications at positions 2, 4, 6, 8, 10, 12, 14, 16, 18, and 20 (counting from the 5′ end); and
(iii) phosphorothioate internucleoside linkages between nucleoside positions 1 and 2, between nucleoside positions 2 and 3, between nucleoside positions 17 and 18, and between nucleoside positions 18 and 19 (counting from the 5′ end).

In any of the above embodiments, the conjugate at the 3′-end of the sense RNAi oligonucleotide may comprise a targeting moiety. In certain such embodiments, the targeting moiety targets a neurotransmitter receptor. In certain embodiments, the cell targeting moiety targets a neurotransmitter transporter. In certain embodiments, the cell targeting moiety targets a GABA transporter. See e.g., WO 2011/131693, WO 2014/064257.

In certain embodiments, the RNAi agent comprises a 21 nucleotide sense RNAi oligonucleotide and a 23 nucleotide antisense RNAi oligonucleotide, wherein the sense RNAi oligonucleotide contains at least one motif of three contiguous 2′-F modified nucleosides at positions 9, 10, 11 from the 5′-end; the antisense RNAi oligonucleotide contains at least one motif of three 2′-O-methyl modifications on three consecutive nucleotides at positions 11, 12, 13 from the 5′ end, wherein one end of the RNAi agent is blunt, while the other end comprises a 2 nucleotide overhang. Preferably, the 2 nucleotide overhang is at the 3′-end of the antisense RNAi oligonucleotide.

In certain embodiments, when the 2 nucleotide overhang is at the 3′-end of the antisense RNAi oligonucleotide, there may be two phosphorothioate internucleoside linkages between the terminal three nucleotides, wherein two of the three nucleotides are the overhang nucleotides, and the third nucleotide is a paired nucleotide next to the overhang nucleotide. In certain embodiments, the RNAi agent additionally has two phosphorothioate internucleoside linkages between the terminal three nucleotides at both the 5′-end of the sense RNAi oligonucleotide and at the 5′-end of the antisense RNAi oligonucleotide. In certain embodiments, every nucleotide in the sense RNAi oligonucleotide and the antisense RNAi oligonucleotide of the RNAi agent is a modified nucleotide. In certain embodiments, each nucleotide is independently modified with a 2′-O-methyl or 3′-fluoro, e.g. in an alternating motif Optionally, the RNAi agent comprises a conjugate.

In certain embodiments, every nucleotide in the sense RNAi oligonucleotide and antisense RNAi oligonucleotide of the RNAi agent, including the nucleotides that are part of the motifs, may be modified. Each nucleotide may be modified with the same or different modification, which can include one or more alteration of one or both of the non-linking phosphate oxygens; alteration of a constituent of the ribose sugar, e.g., of the 2′ hydroxyl on the ribose sugar; wholesale replacement of the phosphate moiety with “dephospho” linkers; modification or replacement of a naturally occurring base; and replacement or modification of the ribose-phosphate backbone.

In certain embodiments, each nucleoside of the sense RNAi oligonucleotide and antisense RNAi oligonucleotide is independently modified with LNA, cEt, UNA, HNA, CeNA, 2′-MOE, 2′-OMe, 2′-O-allyl, 2′-C-allyl, 2′-deoxy, 2′-hydroxyl, or 2′-fluoro. The RNAi agent can contain more than one modification. In one embodiment, each nucleoside of the sense RNAi oligonucleotide and antisense RNAi oligonucleotide is independently modified with 2′-O-methyl or 2′-F. In certain embodiments, the modification is a 2′-NMA modification.

The term “alternating motif” as used herein refers to a motif having one or more modifications, each modification occurring on alternating nucleotides of one RNAi oligonucleotide. The alternating nucleotide may refer to one per every other nucleotide or one per every three nucleotides, or a similar pattern. For example, if A, B and C each represent one type of modification to the nucleotide, the alternating motif can be “ABABABABABAB . . . ,” “AABBAABBAABB . . . ,” “AABAABAABAAB . . . ,” “AAABAAABAAAB . . . ,” “AAABBBAAABBB . . . ,” or “ABCABCABCABC . . . ,” etc.

The type of modifications contained in the alternating motif may be the same or different. For example, if A, B, C, D each represent one type of modification on the nucleotide, the alternating pattern, i.e., modifications on every other nucleotide, may be the same, but each of the sense RNAi oligonucleotide or antisense RNAi oligonucleotide can be selected from several possibilities of modifications within the alternating motif such as “ABABAB . . . ”, “ACACAC . . . ” “BDBDBD . . . ” or “CDCDCD . . . ,” etc.

In certain embodiments, the modification pattern for the alternating motif on the sense RNAi oligonucleotide relative to the modification pattern for the alternating motif on the antisense RNAi oligonucleotide is shifted. The shift may be such that the group of modified nucleotides of the sense RNAi oligonucleotide corresponds to a group of differently modified nucleotides of the antisense RNAi oligonucleotide and vice versa. For example, the sense RNAi oligonucleotide when paired with the antisense RNAi oligonucleotide in the RNAi duplex, the alternating motif in the sense RNAi oligonucleotide may start with “ABABAB” from 5′-3′ of the RNAi oligonucleotide and the alternating motif in the antisense RNAi oligonucleotide may start with “BABABA” from 5′-3′ of the RNAi oligonucleotide within the duplex region. As another example, the alternating motif in the sense RNAi oligonucleotide may start with “AABBAABB” from 5′-3′ of the RNAi oligonucleotide and the alternating motif in the antisense RNAi oligonucleotide may start with “BBAABBAA” from 5′-3′ of the RNAi oligonucleotide within the duplex region, so that there is a complete or partial shift of the modification 10 patterns between the sense RNAi oligonucleotide and the antisense RNAi oligonucleotide.

In certain embodiments, the RNAi agent comprising the pattern of the alternating motif of 2′-O-methyl modification and 2′-F modification on the sense RNAi oligonucleotide initially has a shift relative to the pattern of the alternating motif of 2′-O-methyl modification and 2′-F modification on the antisense RNAi oligonucleotide initially, i.e., the 2′-O-methyl modified nucleotide on the sense RNAi oligonucleotide base pairs with a 2′-F modified nucleotides on the antisense RNAi oligonucleotide and vice versa. The 1 position of the sense RNAi oligonucleotide may start with the 2′-F modification, and the 1 position of the antisense RNAi oligonucleotide may start with a 2′-O-methyl modification.

The introduction of one or more motifs of three identical modifications on three consecutive nucleotides to the sense RNAi oligonucleotide and/or antisense RNAi oligonucleotide interrupts the initial modification pattern present in the sense RNAi oligonucleotide and/or antisense RNAi oligonucleotide. This interruption of the modification pattern of the sense and/or antisense RNAi oligonucleotide by introducing one or more motifs of three identical modifications on three consecutive nucleotides to the sense and/or antisense RNAi oligonucleotide surprisingly enhances the gene silencing activity to the target gene. In one embodiment, when the motif of three identical modifications on three consecutive 25 nucleotides is introduced to any of the RNAi oligonucleotide s, the modification of the nucleotide next to the motif is a different modification than the modification of the motif. For example, the portion of the sequence containing the motif is “ . . . NaYYYNb,” where “Y” represents the modification of the motif of three identical modifications on three consecutive nucleotide, and “Na” and “Nb” represent a modification to the nucleotide next to the motif “YYY” that is different than the modification of Y, and where Na and Nb can be the same or different modifications. Alternatively, Na and/or Nb may be present or absent when there is a wing modification present.

In certain embodiments, the sense RNAi oligonucleotide may be represented by formula (I):

5′nₚ-Nₐ—(X X X)i-N_{b}—Y Y Y—N_{b}—(Z Z Z)ᵣNₐ-n_{q}3′ (I)

wherein:

i and j are each independently 0 or 1;

p and q are each independently 0-6;

each Nₐ independently represents 0-25 linked nucleosides comprising at least two differently modified nucleosides;

each N_{b} independently represents 0-10 linked nucleosides;

each nₚ and n_{q} independently represent an overhanging nucleoside;

wherein N_{b} and Y do not have the same modification; and

XXX, YYY and ZZZ each independently represent modified nucleosides where each X nucleoside has the same modification; each Y nucleoside has the same modification; and each Z nucleoside has the same modification. In certain embodiments, each Y comprises a 2′-F modification.

In certain embodiments, the Nₐ and N_{b} comprise modifications of alternating patterns.

In certain embodiments, the YYY motif occurs at or near the cleavage site of the target nucleic acid. For example, when the RNAi agent has a duplex region of 17-23 nucleotides in length, the YYY motif can occur at or near the vicinity of the cleavage site (e.g., can occur at positions 6, 7, 8; 7, 8, 9; 8, 9, 10; 9, 10, 11; 10, 11, 12; or 11, 12, 13) of the sense RNAi oligonucleotide, the count starting from the 1′ nucleotide from the 5′-end; or optionally, the count starting at the 1′ paired nucleotide within the duplex region, from the 5′-end.

In certain embodiments, the antisense RNAi oligonucleotide of the RNAi may be represented by the formula:

5′n_{q}-Nₐ′—(Z′Z′Z′)ₖ—N_{b}′—Y′Y′Y′—N_{b}′—(X′X′X′)ₗ—N′ₐ-nₚ 3′ (II)

wherein:

k and l are each independently 0 or 1;

p′ and q′ are each independently 0-6;

each Nₐ′ independently represents 0-25 linked nucleotides comprising at least two differently modified nucleotides;

each N_{b}′ independently represents 0-10 linked nucleotides;

each nₚ′ and n′ independently represent an overhanging nucleoside;

wherein N_{b}′ and Y′ do not have the same modification; and

X′X′X′, Y′Y′Y′ and Z′Z′Z′ each independently represent modified nucleosides where each X′ nucleoside has the same modification; each Y′ nucleoside has the same modification; and each Z′ nucleoside has the same modification. In certain embodiments, each Y′ comprises a 2′-F modification. In certain embodiments, each Y′ comprises a 2′-OMe modification.

In certain embodiments, the Nₐ′ and/or N_{b}′ comprise modifications of alternating patterns.

In certain embodiments, the Y′Y′Y′ motif occurs at or near the cleavage site of the target nucleic acid. For example, when the RNAi agent has a duplex region of 17-23 nucleotides in length, the Y′Y′Y′ motif can occur at positions 9, 10, 11; 10, 11, 12; 11, 12, 13; 12, 13, 14; or 13, 14, 15 of the antisense RNAi oligonucleotide, with the count starting from the 1′ nucleotide from the 5′-end; or, optionally, the count starting at the 1′ paired nucleotide within the duplex region, from the 5′-end. Preferably, the Y′Y′Y′ motif occurs at positions 11, 12, 13.

In certain embodiments, k is 1 and 1 is 0, or k is 0 and 1 is 1, or both k and l are 1.

The antisense RNAi oligonucleotide can therefore be represented by the following formulas:

5′n_{q}′—Nₐ′—Z′Z′Z′—N_{b}′—Y′Y′Y′—Nₐ′-nₚ′3′ (IIb);

5′n_{q}′—Nₐ′—Y′Y′Y′—N_{b}′—X′X′X′-nₚ′3′ (IIc); or

5′n_{q}′—Nₐ′—Z′Z′Z′—N_{b}′—Y′Y′Y′—N_{b}′—X′X′X′—Nₐ′-nₚ′3′ (IId).

When the antisense RNAi oligonucleotide is represented by formula IIb, N_{b}′ represents 0-10, 0-7, 0-5, 0-4, 0-2, or 0 linked nucleosides. Each Nₐ′ independently represents 2-20, 2-15, or 2-10 linked nucleosides.

When the antisense RNAi oligonucleotide is represented by formula IIc, N_{b}′ represents 0-10, 0-7, 0-5, 0-4, 0-2, or 0 linked nucleosides. Each Nₐ′ independently represents 2-20, 2-15, or 2-10 linked nucleosides.

When the antisense RNAi oligonucleotide is represented by formula IId, N_{b}′ represents 0-10, 0-7, 0-5, 0-4, 0-2, or 0 linked nucleosides. Each Nₐ′ independently represents 2-20, 2-15, or 2-10 linked nucleosides. Preferably, N_{b}′ is 0, 1, 2, 3, 4, 5, or 6.

In certain embodiments, k is 0 and 1 is 0 and the antisense RNAi oligonucleotide may be represented by the formula:

5′nₚ′—Nₐ′—Y′Y′Y′—Nₐ′-n_{q}′3′ (Ia).

When the antisense RNAi oligonucleotide is represented by formula IIa, each Nₐ′ independently represents 2-20, 2-15, or 2-10 linked nucleosides.

Each X′, Y′, and Z′ may be the same or different from each other.

Each nucleotide of the sense RNAi oligonucleotide and antisense RNAi oligonucleotide may be independently modified with LNA, UNA, cEt, HNA, CeNA, 2′-methoxyethyl, 2′-O-methyl, 2′-O-allyl, 2′-C-allyl, 2′-hydroxyl, or 2′-fluoro. For example, each nucleotide of the sense RNAi oligonucleotide and antisense RNAi oligonucleotide is independently modified with, 2′-O-methyl or 2′-fluoro. Each X, Y, Z, X′, Y′, and Z′, in particular, may represent a 2′-O-methyl modification or 2′-fluoro modification. In certain embodiments, the modification is a 2′-NMA modification.

In certain embodiments, the sense RNAi oligonucleotide of the RNAi agent may contain YYY motif occurring at 9, 10, and 11 positions of the RNAi oligonucleotide when the duplex region is 21 nucleotides, the count starting from the 1′ nucleotide from the 5′-end, or optionally, the count starting at the 1′ paired nucleotide within the duplex region, from the 5′-end; and Y represents 2′-F modification. The sense RNAi oligonucleotide may additionally contain XXX motif or ZZZ motifs as wing modifications at the opposite end of the duplex region; and XXX and ZZZ each independently represents a 2′-O-methyl modification or 2′-fluoro modification.

In certain embodiments, the antisense RNAi oligonucleotide may contain Y′Y′Y′ motif occurring at positions 11, 12, 13 of the RNAi oligonucleotide, the count starting from the 1^{st} nucleotide from the 5′-end, or optionally, the count starting at the 1′ paired nucleotide within the duplex region, from the 5′-end; and Y′ represents 2′-O-methyl modification. The antisense RNAi oligonucleotide may additionally contain X′X′X′ motif or Z′Z′Z′ motif as wing modifications at the opposite end of the duplex region; and X′X′X′ or Z′Z′Z′ each independently represents a 2′-O-methyl modification or 2′-fluoro modification.

The sense RNAi oligonucleotide represented by any one of the above formulas Ia, Ib, Ic, and Id forms a duplex with an antisense RNAi oligonucleotide being represented by any one of the formulas IIa, IIb, IIc, and IId, respectively.

Accordingly, the RNAi agents described herein may comprise a sense RNAi oligonucleotide and an antisense RNAi oligonucleotide, each RNAi oligonucleotide having 14 to 30 nucleotides, the RNAi duplex represented by formula (III):

Sense:
5′ nₚ-Nₐ-(XXX)ᵢ-N_{b}-YYY-N_{b}-(ZZZ)ⱼ-Nₐ-n_{q} 3′
Antisense:
3′ nₚ′-Nₐ′-(X′X′X′)ₖ-N_{b}′-Y′Y′Y′-N_{b}′-(Z′Z′Z′)ₗ-Nₐ′-
n_{q}′ 5′

wherein:

i, j, k, and l are each independently 0 or 1;

p, p′, q, and q′ are each independently 0-6;

each Nₐ and Nₐ′ independently represents 0-25 linked nucleosides, each sequence comprising at least two differently modified nucleotides;

each N_{b} and N_{b}′ independently represents 0-10 linked nucleosides;

wherein each nₚ′, nₚ, n_{q}′ and n_{q}, each of which may or may not be present, independently represents an overhang nucleotide; and

XXX, YYY, X′X′X′, Y′Y′Y′, and Z′Z′Z′ each independently represent one motif of three identical modifications on three consecutive nucleotides.

In certain embodiments, i is 0 and j is 0; or i is 1 and j is 0; or i is 0 and j is 1; or both i and j are 0; or both i and j are 1. In another embodiment, k is 0 and 1 is 0; or k is 1 and 1 is 0, or k is 0 and 1 is 1; or both k and 1 are 0; or both k and l are 1.

Exemplary combinations of the sense RNAi oligonucleotide and antisense RNAi oligonucleotide forming a RNAi duplex include the formulas below:

5′nₚ-Nₐ—Y Y Y—Nₐ-n_{q} 3′

3′ np′-Nₐ′—Y′Y′Y′—Nₐ′n_{q}′ 5′ (IIIa)

5′nₚ-Nₐ—Y Y Y—N_{b}—Z Z Z—Nₐ-n_{q}3′

3′nₚ′—Nₐ′—Y′Y′Y′—Nb′—Z′Z′Z′—Nₐ′n_{q}'5′ (IIIb)

5′np-Nₐ—X X X—N_{b}—Y Y Y—Nₐ-n_{q} 3′

3′ np′-Nₐ′—X′X′X′—N_{b}′—Y′Y′Y′—Nₐ′-n_{q}′ 5′ (IIIc)

5′np-Nₐ—X X X—N_{b}—Y Y Y—N_{b}—Z Z Z—Nₐ-n_{q}3′

3′ np′-Nₐ′—X′X′X′—N_{b}′—Y′Y′Y′—N_{b}′—Z′Z′Z′—Nₐ-n_{q}'5′ (IIId)

When the RNAi agent is represented with formula IIIa, each Nₐ independently represents 2-20, 2-15, or 2-10 linked nucleosides.

When the RNAi agent is represented with formula IIIb, each N_{b} independently represents 1-10, 1-7, 1-5, or 1-4 linked nucleosides. Each Nₐ independently represents 2-20, 2-15, or 2-10 linked nucleosides.

When the RNAi agent is represented with formula IIIc, each N_{b}, N_{b}′ independently represents 0-10, 0-7, 0-10, 0-7, 0-5, 0-4, 0-2, or 0 linked nucleosides. Each Nₐ independently represents 2-20, 2-15, or 2-10 linked nucleosides.

When the RNAi agent is represented with formula IIId, each N_{b}, N_{b}′ independently represents 0-10, 0-7, 0-10, 0-7, 0-5, 0-4, 0-2, or 0 linked nucleosides. Each Nₐ, Nₐ′ independently 2-20, 2-15, or 2-10 linked nucleosides. Each Nₐ, Nₐ′, N_{b}, N_{b}′ independently comprises modifications of alternating pattern.

Each of X, Y, and Z in formulas III, IIIa, IIIb, IIIc, and IIId may be the same or different from each other.

When the RNAi agent is represented by formula III, IIIa, IIIb, IIIc, and/or IIId, at least one of the Y nucleotides may form a base pair with one of the Y′ nucleotides. Alternatively, at least two of the Y nucleotides may form base pairs with the corresponding Y′ nucleotides; or all three of the Y nucleotides may form base pairs with the corresponding Y′ nucleotides.

When the RNAi agent is represented by formula IIIb or IIId, at least one of the Z nucleotides may form a base pair with one of the Z′ nucleotides. Alternatively, at least two of the Z nucleotides may form base pairs with the corresponding Z′ nucleotides; or all three of the Z nucleotides may form base pairs with the corresponding Z′ nucleotides.

When the RNAi agent is represented by formula IIIc or IIId, at least one of the X nucleotides may form a base pair with one of the X′ nucleotides. Alternatively, at least two of the X nucleotides may form base pairs with the corresponding X′ nucleotides; or all three of the X nucleotides may form base pairs with the corresponding X′ nucleotides.

In certain embodiments, the modification of the Y nucleotide is different than the modification on the Y′ nucleotide, the modification on the Z nucleotide is different than the modification on the Z′ nucleotide, and/or the modification on the X nucleotide is different than the modification on the X′ nucleotide.

In certain embodiments, when the RNAi agent is represented by the formula IIId, the Nₐ modifications are 2′-O-methyl or 2′-fluoro modifications. In another embodiment, when the RNAi agent is represented by formula IIId, the Nₐ modifications are 2′-O-methyl or 2′-fluoro modifications and nₚ′>0 and at least one nₚ′ is linked to a neighboring nucleotide via phosphorothioate linkage. In other embodiments, when the RNAi agent is represented by formula IIId, the Nₐ modifications are 2′-O-methyl or 2′-fluoro modifications, nₚ′>0 and at least one nₚ′ is linked to a neighboring nucleotide via phosphorothioate linkage, and the sense RNAi oligonucleotide is conjugated to one or more cell targeting group attached through a bivalent or trivalent branched linker. In certain embodiments, when the RNAi agent is represented by formula IIId, the Nₐ modifications are 2′-O-methyl or 2′-fluoro modifications, nₚ′>0 and at least one nₚ′ is linked to a neighboring nucleotide via phosphorothioate linkage, the sense RNAi oligonucleotide comprises at least one phosphorothioate linkage and the sense RNAi oligonucleotide is conjugated to one or more cell targeting group attached through a bivalent or trivalent branched linker.

In certain embodiments, when the RNAi agent is represented by the formula IIIa, the Nₐ modifications are 2′-O-methyl or 2′-fluoro modifications and nₚ′>0 and at least one nₚ′ is linked to a neighboring nucleotide via phosphorothioate linkage, the sense RNAi oligonucleotide comprises at least one phosphorothioate linkage and the sense RNAi oligonucleotide is conjugated to one or more cell targeting group attached through a bivalent or trivalent branched linker.

In certain embodiments, the modification is a 2′-NMA modification.

In certain embodiments, the antisense strand may comprise a stabilized phosphate group attached to the 5′ position of the 5′-most nucleoside. In certain embodiments, the stabilized phosphate group comprises an (E)-vinyl phosphonate. In certain embodiments, the stabilized phosphate group comprises a cyclopropyl phosphonate.

In certain embodiments, the antisense strand may comprise a seed-pairing destabilizing modification. In certain embodiments, the seed-pairing destabilizing modification is located at position 6 (counting from the 5′ end). In certain embodiments, the seed-pairing destabilizing modification is located at position 7 (counting from the 5′ end). In certain embodiments, the seed-pairing destabilizing modification is a GNA sugar surrogate. In certain embodiments, the seed-pairing destabilizing modification is an (S)-GNA. In certain embodiments, the seed-pairing destabilizing modification is a UNA. In certain embodiments, the seed-pairing destabilizing modification is a morpholino.

In certain embodiments, the sense strand may comprise an inverted abasic sugar moiety attached to the 5′-most nucleoside. In certain embodiments, the sense strand may comprise an inverted abasic sugar moiety attached to the 3′-most nucleoside. In certain embodiments, the sense strand may comprise inverted abasic sugar moieties attached to both the 5′-most and 3′-most nucleosides.

In certain embodiments, the sense strand may comprise a conjugate attached at position 6 (counting from the 5′ end). In certain embodiments, the conjugate is attached at the 2′ position of the nucleoside. In certain embodiments the conjugate is a C₁₆ lipid conjugate. In certain embodiments, the modified nucleoside at position 6 of the sense strand has a 2′-O-hexadecyl modified sugar moiety.

III. Oligomeric Duplexes

In certain embodiments, oligomeric compounds described herein comprise an oligonucleotide, having a nucleobase sequence complementary to that of a target nucleic acid. In certain embodiments, an oligomeric compound is paired with a second oligomeric compound to form an oligomeric duplex. Such oligomeric duplexes comprise a first oligomeric compound having a region complementary to a target nucleic acid and a second oligomeric compound having a region complementary to the first oligomeric compound. In certain embodiments, the first oligomeric compound of an oligomeric duplex comprises or consists of (1) a modified or unmodified oligonucleotide and optionally a conjugate group and (2) a second modified or unmodified oligonucleotide and optionally a conjugate group. Either or both oligomeric compounds of an oligomeric duplex may comprise a conjugate group. The oligonucleotides of each oligomeric compound of an oligomeric duplex may include non-complementary overhanging nucleosides.

IV. Antisense Activity

In certain embodiments, oligomeric compounds and oligomeric duplexes are capable of hybridizing to a target nucleic acid, resulting in at least one antisense activity; such oligomeric compounds and oligomeric duplexes are antisense compounds. In certain embodiments, antisense compounds have antisense activity when they reduce or inhibit the amount or activity of a target nucleic acid by 25% or more in a standard cell assay. In certain embodiments, antisense compounds selectively affect one or more target nucleic acid. Such antisense compounds comprise a nucleobase sequence that hybridizes to one or more target nucleic acid, resulting in one or more desired antisense activity and does not hybridize to one or more non-target nucleic acid or does not hybridize to one or more non-target nucleic acid in such a way that results in significant undesired antisense activity.

In certain antisense activities, hybridization of an antisense compound to a target nucleic acid results in recruitment of a protein that cleaves the target nucleic acid. For example, certain antisense compounds result in RNase H mediated cleavage of the target nucleic acid. RNase H is a cellular endonuclease that cleaves the RNA strand of an RNA:DNA duplex. The DNA in such an RNA:DNA duplex need not be unmodified DNA. In certain embodiments, described herein are antisense compounds that are sufficiently “DNA-like” to elicit RNase H activity. In certain embodiments, one or more non-DNA-like nucleoside in the gap of a gapmer is tolerated.

In certain antisense activities, an antisense compound or a portion of an antisense compound is loaded into an RNA-induced silencing complex (RISC), ultimately resulting in cleavage of the target nucleic acid. For example, certain antisense compounds result in cleavage of the target nucleic acid by Argonaute. Antisense compounds that are loaded into RISC are RNAi compounds. RNAi compounds may be double-stranded (siRNA) or single-stranded (ssRNA).

In certain embodiments, hybridization of an antisense compound to a target nucleic acid does not result in recruitment of a protein that cleaves that target nucleic acid. In certain embodiments, hybridization of the antisense compound to the target nucleic acid results in alteration of splicing of the target nucleic acid. In certain embodiments, hybridization of an antisense compound to a target nucleic acid results in inhibition of a binding interaction between the target nucleic acid and a protein or other nucleic acid. In certain embodiments, hybridization of an antisense compound to a target nucleic acid results in alteration of translation of the target nucleic acid.

Antisense activities may be observed directly or indirectly. In certain embodiments, observation or detection of an antisense activity involves observation or detection of a change in an amount of a target nucleic acid or protein encoded by such target nucleic acid, a change in the ratio of splice variants of a nucleic acid or protein and/or a phenotypic change in a cell or subject.

V. Certain Target Nucleic Acids

In certain embodiments, oligomeric compounds comprise or consist of an oligonucleotide comprising a region that is complementary to a target nucleic acid. In certain embodiments, the target nucleic acid is an endogenous RNA molecule. In certain embodiments, the target nucleic acid encodes a protein. In certain such embodiments, the target nucleic acid is selected from: a mature mRNA and a pre-mRNA, including intronic, exonic and untranslated regions. In certain embodiments, the target RNA is a mature mRNA. In certain embodiments, the target nucleic acid is a pre-mRNA. In certain such embodiments, the target region is entirely within an intron. In certain embodiments, the target region spans an intron/exon junction. In certain embodiments, the target region is at least 50% within an intron. In certain embodiments, the target nucleic acid is the RNA transcriptional product of a retrogene. In certain embodiments, the target nucleic acid is a non-coding RNA. In certain such embodiments, the target non-coding RNA is selected from: a long non-coding RNA, a short non-coding RNA, an intronic RNA molecule.

A. Complementarity/Mismatches to the Target Nucleic Acid and Duplex Complementarity

### Gapmer Oligonucleotides

It is possible to introduce mismatch bases without eliminating activity. For example, Gautschi et al (J. Natl. Cancer Inst. 93:463-471, March 2001) demonstrated the ability of an oligonucleotide having 100% complementarity to the bcl-2 mRNA and having 3 mismatches to the bcl-xL mRNA to reduce the expression of both bcl-2 and bcl-xL in vitro and in vivo. Furthermore, this oligonucleotide demonstrated potent anti-tumor activity in vivo. Maher and Dolnick (Nuc. Acid. Res. 16:3341-3358, 1988) tested a series of tandem 14 nucleobase oligonucleotides, and a 28 and 42 nucleobase oligonucleotides comprised of the sequence of two or three of the tandem oligonucleotides, respectively, for their ability to arrest translation of human DHFR in a rabbit reticulocyte assay. Each of the three 14 nucleobase oligonucleotides alone was able to inhibit translation, albeit at a more modest level than the 28 or 42 nucleobase oligonucleotides.

In certain embodiments, oligonucleotides are complementary to the target nucleic acid over the entire length of the oligonucleotide. In certain embodiments, oligonucleotides are 99%, 95%, 90%, 85%, or 80% complementary to the target nucleic acid. In certain embodiments, oligonucleotides are at least 80% complementary to the target nucleic acid over the entire length of the oligonucleotide and comprise a region that is 100% or fully complementary to a target nucleic acid. In certain embodiments, the region of full complementarity is from 6 to 20, 10 to 18, or 18 to 20 nucleobases in length.

In certain embodiments, oligonucleotides comprise one or more mismatched nucleobases relative to the target nucleic acid. In certain embodiments, antisense activity against the target is reduced by such mismatch, but activity against a non-target is reduced by a greater amount. Thus, in certain embodiments selectivity of the oligonucleotide is improved. In certain embodiments, the mismatch is specifically positioned within an oligonucleotide having a gapmer motif. In certain embodiments, the mismatch is at position 1, 2, 3, 4, 5, 6, 7, or 8 from the 5′-end of the gap region. In certain embodiments, the mismatch is at position 9, 8, 7, 6, 5, 4, 3, 2, 1 from the 3′-end of the gap region. In certain embodiments, the mismatch is at position 1, 2, 3, or 4 from the 5′-end of the wing region. In certain embodiments, the mismatch is at position 4, 3, 2, or 1 from the 3′-end of the wing region.

### Antisense RNAi Oligonucleotides

In certain embodiments, antisense RNAi oligonucleotides comprise one or more mismatched nucleobases relative to the target nucleic acid. In certain embodiments, RNAi activity against the target is reduced by such mismatch, but activity against a non-target is reduced by a greater amount. Thus, in certain embodiments selectivity of the antisense RNAi oligonucleotides is improved.

In certain embodiments, antisense RNAi oligonucleotides comprise a targeting region complementary to the target nucleic acid. In certain embodiments, the targeting region comprises or consists of at least 8, at least 9, at least 10, at least 11, at least 12, at least 13, at least 14, at least 15, at least 16, at least 17, at least 18, at least 19, at least 20, at least 21, at least 22, at least 23, at least 25 or at least 25 contiguous nucleotides. In certain embodiments, the targeting region constitutes 70%, 80%, 85%, 90%, 95% of the nucleosides of the antisense RNAi oligonucleotide. In certain embodiments, the targeting region constitutes all of the nucleosides of the antisense RNAi oligonucleotide. In certain embodiments, the targeting region of the antisense RNAi oligonucleotide is at least 99%, 95%, 90%, 85%, or 80% complementary to the target nucleic acid. In certain embodiments, the targeting region of the antisense RNAi oligonucleotide is 100% complementary to the target nucleic acid.

### Sense RNAi Oligonucleotides

In certain embodiments, RNAi agents comprise a sense RNAi oligonucleotide. In such embodiments, sense RNAi oligonucleotides comprise an antisense hybridizing region complementary to the antisense RNAi oligonucleotide. In certain embodiments, the antisense hybridizing region comprises or consists of at least 8, at least 9, at least 10, at least 11, at least 12, at least 13, at least 14, at least 15, at least 16, at least 17, at least 18, at least 19, at least 20, at least 21, at least 22, at least 23, at least 25 or at least 25 contiguous nucleotides. In certain embodiments, the antisense hybridizing region constitutes 70%, 80%, 85%, 90%, 95% of the nucleosides of the sense RNAi oligonucleotide. In certain embodiments, the antisense hybridizing region constitutes all of the nucleosides of the sense RNAi oligonucleotide. In certain embodiments, the antisense hybridizing region of the sense RNAi oligonucleotide is at least 99%, 95%, 90%, 85%, or 80% complementary to the antisense RNAi oligonucleotide. In certain embodiments, the antisense hybridizing region of the sense RNAi oligonucleotide is 100% complementary to the antisense RNAi oligonucleotide.

The hybridizing region of a sense RNAi oligonucleotide hybridizes with the antisense RNAi oligonucleotide to form a duplex region. In certain embodiments, such duplex region consists of 7 hybridized pairs of nucleosides (one of each pair being on the antisense RNAi oligonucleotide and the other of each pair bien on the sense RNAi oligonucleotide). In certain embodiments, a duplex region comprises least 8, at least 9, at least 10, at least 11, at least 12, at least 13, at least 14, at least 15, at least 16, at least 17, at least 18, at least 19, at least 20, at least 21, at least 22, at least 23, at least 25 or at least 25 hybridized pairs. In certain embodiments, each nucleoside of antisense RNAi oligonucleotide is paired in the duplex region (i.e., the antisense RNAi oligonucleotide has no overhanging nucleosides). In certain embodiments, the antisense RNAi oligonucleotide includes unpaired nucleosides at the 3′-end and/or the 5′end (overhanging nucleosides). In certain embodiments, each nucleoside of sense RNAi oligonucleotide is paired in the duplex region (i.e., the sense RNAi oligonucleotide has no overhanging nucleosides). In certain embodiments, the sense RNAi oligonucleotide includes unpaired nucleosides at the 3′-end and/or the 5′end (overhanging nucleosides). In certain embodiments, duplexes formed by the antisense RNAi oligonucleotide and the sense RNAi oligonucleotide do not include any overhangs at one or both ends. Such ends without overhangs are referred to as blunt. In certain embodiments wherein the antisense RNAi oligonucleotide has overhanging nucleosides, one or more of those overhanging nucleosides are complementary to the target nucleic acid. In certain embodiments wherein the antisense RNAi oligonucleotide has overhanging nucleosides, one or more of those overhanging nucleosides are not complementary to the target nucleic acid.

B. SPDEF

In certain embodiments, oligomeric compounds comprise or consist of an oligonucleotide comprising a region that is complementary to a SPDEF nucleic acid. In certain embodiments, the SPDEF nucleic acid has the sequence set forth in SEQ ID NO: 1 (GENBANK Accession No. NM_012391.2). In certain embodiments, the SPDEF nucleic acid has the sequence set forth in SEQ ID NO: 2 (the complement of GENBANK Accession No. NC_000006.12 truncated from nucleotides 34536001 to 34558000). In certain embodiments, the SPDEF nucleic acid has the sequence set forth in SEQ ID NO: 3 (GENBANK Accession No. NM_001252294.1). In certain embodiments, the SPDEF nucleic acid has the sequence set forth in SEQ ID NO: 4 (GENBANK Accession No. XM_005248988.3). In certain embodiments, the SPDEF nucleic acid has the sequence set forth in SEQ ID NO: 5 (GENBANK Accession No. XM_006715048.1).

In certain embodiments an oligomeric compound complementary to any one of SEQ ID NOS: 1-5 is capable of reducing an SPDEF RNA in a cell. In certain embodiments an oligomeric compound complementary to any one of SEQ ID NOS: 1-5 is capable of reducing an SPDEF protein in a cell. In certain embodiments, the cell is in vitro. In certain embodiments, the cell is in a subject. In certain embodiments, the oligomeric compound consists of a modified oligonucleotide.

In certain embodiments, an oligomeric compound complementary to any one of SEQ ID NOS: 1-5 is capable of ameliorating one or more symptoms or hallmarks of a pulmonary condition when it is introduced to a cell in a subject. In certain embodiments, the one or more symptoms or hallmarks are selected from shortness of breath, chest pain, coughing, wheezing, fatigue, sleep disruption, bronchospasm, and combinations thereof. In certain embodiments, the pulmonary condition is selected from bronchitis, asthma, COPD, pneumonia, emphysema, rhinitis, sinusitis, nasal polyposis, sinus polyposis, bronchiectasis, and sarcoidosis. In certain embodiments, the pulmonary condition is chronic bronchitis. Chronic bronchitis may be characterized by a cough productive of sputum for over three months' duration for two consecutive years. In certain embodiments, the pulmonary condition is a result of an allergic reaction. In certain embodiments, the pulmonary condition is a result of a viral infection. For example, the pulmonary condition may be a common cold, croup, bronchitis or pneumonia caused by an adenovirus infection. In certain embodiments, the pulmonary condition is severe asthma. In certain embodiments, the pulmonary condition is Type 2 asthma, also referred to as Th2 asthma.

In certain embodiments, an oligomeric compound complementary to any one of SEQ ID NOS: 1-5 is capable of ameliorating one or more symptoms or hallmarks of a gastrointestinal condition when it is introduced to a cell in a subject. In certain embodiments, the gastrointestinal condition is characterized by mucus in the stool of the subject. In certain embodiments, the gastrointestinal condition is ulcerative colitis.

In certain embodiments, an oligomeric compound complementary to any one of SEQ ID NOS: 1-5 is capable of reducing a detectable amount of an SPDEF RNA in the lung of a subject when the oligomeric compound is administered to the subject. In some instances, the oligomeric compound is administered via an inhaler or nebulizer. The detectable amount of the SPDEF RNA may be reduced by at least 10%, at least 20%, at least 30%, at least 40%, at least 50%, at least 60%, at least 70%, at least 80%, or at least 90%. In certain embodiments, an oligomeric compound complementary to any one of SEQ ID NOS: 1-5 is capable of reducing a detectable amount of an SPDEF protein in the lung of the subject when the oligomeric compound is administered to the subject. The detectable amount of the SPDEF protein may be reduced by at least 10%, at least 20%, at least 30%, at least 40%, at least 50%, at least 60%, at least 70%, at least 80%, or at least 90%.

VI. Certain Compounds

1. Compound No. 833561

In certain embodiments, the oligomeric compound is Compound No. 833561. In certain embodiments, Compound No. 833561 is characterized as an oligomeric compound consisting of a modified oligonucleotide, wherein the modified oligonucleotide is a 3-10-3 cEt gapmer, having a sequence of (from 5′ to 3′) CAATAAGCAAGTCTGG (SEQ ID NO: 1129), wherein each of nucleosides 1-3 and 14-16 (from 5′ to 3′) comprise a cEt modification and each of nucleosides 4-13 are 2′-deoxynucleosides, wherein the internucleoside linkages between all nucleosides are phosphorothioate linkages, and wherein each cytosine is a 5-methyl cytosine.

In certain embodiments, Compound 833561 is characterized by the following chemical notation: mCks Aks Aks Tds Ads Ads Gds mCds Ads Ads Gds Tds mCds Tks Gks Gk; wherein

A=an adenine nucleobase

mC=a 5-methyl cytosine nucleobase

G=a guanine nucleobase

T=a thymine nucleobase

k=a cEt modified sugar

d=a 2′-deoxyribose sugar, and

s=a phosphorothioate internucleoside linkage.

In certain embodiments, Compound No. 833561 is represented by the following chemical structure:

[chemical structure]

In certain embodiments, Compound No. 833561 is in the form of an anion or a salt thereof. For example, the oligomeric compound may be in the form of a sodium salt. In certain embodiments, the oligomeric compound is in anionic form in a solution.

In certain embodiments, Compound No. 833561 is represented by the following chemical structure:

[chemical structure]

In certain embodiments, Compound No. 833561 is represented by the following chemical structure:

[chemical structure]

### Compound No. 936142

In certain embodiments, the oligomeric compound is Compound No. 936142. In certain embodiments, Compound No. 936142 is characterized as an oligomeric compound consisting of a modified oligonucleotide, wherein the modified oligonucleotide is a 2-9-5 mixed-wing cEt/MOE gapmer, having a sequence of ACTTGTAACAGTGGTT (from 5′ to 3′) (SEQ ID NO: 1983), wherein each of nucleosides 1-2 and 15-16 (from 5′ to 3′) comprise a cEt modification, each of nucleosides 12-14 is a 2′-MOE nucleoside, and each of nucleosides 3-11 is a 2′-deoxynucleoside, wherein the internucleoside linkages between all nucleosides are phosphorothioate linkages, and wherein each cytosine is a 5-methyl cytosine.

In certain embodiments, Compound No. 936142 is characterized by the following chemical notation: Aks mCks Tds Tds Gds Tds Ads Ads mCds Ads Gds Tes Ges Ges Tks Tk; wherein

A=an adenine nucleobase

mC=a 5-methyl cytosine nucleobase

G=a guanine nucleobase

T=a thymine nucleobase

k=a cEt modified sugar

d=a 2′-deoxyribose sugar, and

s=a phosphorothioate internucleoside linkage.

In certain embodiments, Compound No. 936142 is represented by the following chemical structure:

[chemical structure]

In certain embodiments, Compound No. 936142 is in the form of an anion or a salt thereof. For example, the oligomeric compound may be in the form of a sodium salt. In certain embodiments, the oligomeric compound is in anionic form in a solution.

In certain embodiments, Compound No. 936142 is characterized by the following chemical structure:

[chemical structure]

VII. Certain Pharmaceutical Compositions & Delivery Systems

In certain embodiments, described herein are pharmaceutical compositions comprising one or more oligomeric compounds. In certain embodiments, the one or more oligomeric compounds each consists of a modified oligonucleotide. In certain embodiments, the pharmaceutical composition comprises a pharmaceutically acceptable diluent or carrier. In certain embodiments, a pharmaceutical composition comprises or consists of a sterile saline solution and one or more oligomeric compound. In certain embodiments, the sterile saline is pharmaceutical grade saline. In certain embodiments, a pharmaceutical composition comprises or consists of one or more oligomeric compound and sterile water. In certain embodiments, the sterile water is pharmaceutical grade water. In certain embodiments, a pharmaceutical composition comprises or consists of one or more oligomeric compound and phosphate-buffered saline (PBS). In certain embodiments, the sterile PBS is pharmaceutical grade PBS. In certain embodiments, a pharmaceutical composition comprises or consists of one or more oligomeric compound and artificial cerebrospinal fluid. In certain embodiments, the artificial cerebrospinal fluid is pharmaceutical grade.

In certain embodiments, pharmaceutical compositions comprise one or more oligomeric compound and one or more excipients. In certain embodiments, excipients are selected from water, salt solutions, alcohol, polyethylene glycols, gelatin, lactose, amylase, magnesium stearate, talc, silicic acid, viscous paraffin, hydroxymethylcellulose and polyvinylpyrrolidone.

In certain embodiments, oligomeric compounds may be admixed with pharmaceutically acceptable active and/or inert substances for the preparation of pharmaceutical compositions or formulations. Compositions and methods for the formulation of pharmaceutical compositions depend on a number of criteria, including, but not limited to, route of administration, extent of disease, or dose to be administered.

In certain embodiments, pharmaceutical compositions comprising an oligomeric compound encompass any pharmaceutically acceptable salts of the oligomeric compound, esters of the oligomeric compound, or salts of such esters. In certain embodiments, pharmaceutical compositions comprising oligomeric compounds comprising one or more oligonucleotide, upon administration to a subject, including a human, are capable of providing (directly or indirectly) the biologically active metabolite or residue thereof. Accordingly, for example, the disclosure is also drawn to pharmaceutically acceptable salts of oligomeric compounds, prodrugs, pharmaceutically acceptable salts of such prodrugs, and other bioequivalents. Suitable pharmaceutically acceptable salts include, but are not limited to, sodium and potassium salts. In certain embodiments, prodrugs comprise one or more conjugate group attached to an oligonucleotide, wherein the conjugate group is cleaved by endogenous nucleases within the body.

Lipid moieties have been used in nucleic acid therapies in a variety of methods. In certain such methods, the nucleic acid, such as an oligomeric compound, is introduced into preformed liposomes or lipoplexes made of mixtures of cationic lipids and neutral lipids. In certain methods, DNA complexes with mono- or poly-cationic lipids are formed without the presence of a neutral lipid. In certain embodiments, a lipid moiety is selected to increase distribution of a pharmaceutical agent to a particular cell or tissue. In certain embodiments, a lipid moiety is selected to increase distribution of a pharmaceutical agent to fat tissue. In certain embodiments, a lipid moiety is selected to increase distribution of a pharmaceutical agent to muscle tissue.

In certain embodiments, pharmaceutical compositions comprise a delivery system. Examples of delivery systems include, but are not limited to, liposomes and emulsions. Certain delivery systems are useful for preparing certain pharmaceutical compositions including those comprising hydrophobic compounds. In certain embodiments, certain organic solvents such as dimethylsulfoxide are used.

In certain embodiments, pharmaceutical compositions comprise one or more tissue-specific delivery molecules designed to deliver the one or more pharmaceutical agents of the present invention to specific tissues or cell types. For example, in certain embodiments, pharmaceutical compositions include liposomes coated with a tissue-specific antibody.

In certain embodiments, pharmaceutical compositions comprise a co-solvent system. Certain of such co-solvent systems comprise, for example, benzyl alcohol, a nonpolar surfactant, a water-miscible organic polymer, and an aqueous phase. In certain embodiments, such co-solvent systems are used for hydrophobic compounds. A non-limiting example of such a co-solvent system is the VPD co-solvent system, which is a solution of absolute ethanol comprising 3% w/v benzyl alcohol, 8% w/v of the nonpolar surfactant Polysorbate 80™ and 65% w/v polyethylene glycol 300. The proportions of such co-solvent systems may be varied considerably without significantly altering their solubility and toxicity characteristics. Furthermore, the identity of co-solvent components may be varied: for example, other surfactants may be used instead of Polysorbate 80™; the fraction size of polyethylene glycol may be varied; other biocompatible polymers may replace polyethylene glycol, e.g., polyvinyl pyrrolidone; and other sugars or polysaccharides may substitute for dextrose.

In certain embodiments, pharmaceutical compositions are prepared for oral administration. In certain embodiments, pharmaceutical compositions are prepared for buccal administration. In certain embodiments, a pharmaceutical composition is prepared for administration by injection (e.g., intravenous, subcutaneous, intramuscular, intrathecal (IT), intracerebroventricular (ICV), etc.). In certain of such embodiments, a pharmaceutical composition comprises a carrier and is formulated in aqueous solution, such as water or physiologically compatible buffers such as Hanks's solution, Ringer's solution, or physiological saline buffer. In certain embodiments, other ingredients are included (e.g., ingredients that aid in solubility or serve as preservatives). In certain embodiments, injectable suspensions are prepared using appropriate liquid carriers, suspending agents and the like. Certain pharmaceutical compositions for injection are presented in unit dosage form, e.g., in ampoules or in multi-dose containers. Certain pharmaceutical compositions for injection are suspensions, solutions or emulsions in oily or aqueous vehicles, and may contain formulatory agents such as suspending, stabilizing and/or dispersing agents. Certain solvents suitable for use in pharmaceutical compositions for injection include, but are not limited to, lipophilic solvents and fatty oils, such as sesame oil, synthetic fatty acid esters, such as ethyl oleate or triglycerides, and liposomes.

Certain embodiments provide pharmaceutical compositions suitable for aerosolization and/or dispersal by a nebulizer or inhaler. In certain embodiments, the pharmaceutical composition is a solid comprising particles of compounds that are of respirable size. A solid particulate composition can optionally contain a dispersant which serves to facilitate the formation of an aerosol, e.g., lactose. Solid pharmaceutical compositions comprising an oligonucleotide can also be aerosolized using any solid particulate medicament aerosol generator known in the art, e.g., a dry powder inhaler. In certain embodiments, the powder employed in the inhaler consists of the compound comprising the active compound or of a powder blend comprising the active compound, a suitable powder diluent, and an optional surfactant. In certain embodiments, the pharmaceutical composition is a liquid. In certain such embodiments, the liquid is administered as an aerosol that is produced by any suitable means, such as with a nebulizer or inhaler. See, e.g., U.S. Pat. No. 4,501,729. In certain embodiments, the nebulizer is a device for producing a spray of liquid. Nebulizers are devices that transform solutions or suspensions into an aerosol mist and are well known in the art. Suitable nebulizers include jet nebulizers, ultrasonic nebulizers, electronic mesh nebulizers, and vibrating mesh nebulizers. In certain embodiments, the nebulizer is activated manually by squeezing a flexible bottle that contains the pharmaceutical composition. In certain embodiments, the aerosol is produced by a metered dose inhaler, which typically contains a suspension or solution formulation of the active compound in a liquefied propellant. Pharmaceutical compositions suitable for aerosolization can comprise propellants, surfactants, co-solvents, dispersants, preservatives, and/or other additives or excipients.

A compound described herein complementary to an SPDEF nucleic acid can be utilized in pharmaceutical compositions by combining the compound with a suitable pharmaceutically acceptable diluent or carrier and/or additional components such that the pharmaceutical composition is suitable for aerosolization by a nebulizer or inhaler. In certain embodiments, a pharmaceutically acceptable diluent is phosphate buffered saline. Accordingly, in one embodiment, employed in the methods described herein is a pharmaceutical composition comprising a compound complementary to an SPDEF nucleic acid and a pharmaceutically acceptable diluent. In certain embodiments, the pharmaceutically acceptable diluent is phosphate buffered saline. In certain embodiments, the compound comprises or consists of a modified oligonucleotide provided herein.

Pharmaceutical compositions comprising compounds provided herein encompass any pharmaceutically acceptable salts, esters, or salts of such esters, or any other oligonucleotide which, upon administration to an animal, including a human, is capable of providing (directly or indirectly) the biologically active metabolite or residue thereof. In certain embodiments, the compounds are antisense compounds or oligomeric compounds. In certain embodiments, the compound comprises or consists of a modified oligonucleotide. Accordingly, for example, the disclosure is also drawn to pharmaceutically acceptable salts of compounds, prodrugs, pharmaceutically acceptable salts of such prodrugs, and other bioequivalents. Suitable pharmaceutically acceptable salts include, but are not limited to, sodium and potassium salts. A prodrug can include the incorporation of additional nucleosides at one or both ends of a compound which are cleaved by endogenous nucleases within the body, to form the active compound.

Under certain conditions, certain compounds disclosed herein are shown in the form of a free acid. Although such compounds may be drawn or described in protonated (free acid) form, aqueous solutions of such compounds may exist in equilibrium among an ionized (anion) form, and in association with a cation (salt form). For example, a phosphate linkage of an oligonucleotide in aqueous solution exists in equilibrium among free acid, anion, and salt forms. Unless otherwise indicated, compounds described herein are intended to include all such forms. Moreover, oligonucleotides have several such linkages, each of which is in equilibrium. Thus, oligonucleotides in solution exist in an ensemble of forms at multiple positions, all at equilibrium. The term “oligonucleotide” is intended to include all such forms. Drawn structures necessarily depict a single form. Nevertheless, unless otherwise indicated, such drawings are likewise intended to include corresponding forms. Herein, a structure depicting the free acid of a compound followed by the term or salts thereof expressly includes all such forms that may be fully or partially protonated/de-protonated/in association with a cation. In certain instances, one or more specific cation is identified.

In certain embodiments, oligomeric compounds disclosed herein are in a form of a sodium salt. In certain embodiments, oligomeric compounds disclosed herein are in a form of a potassium salt. In certain embodiments, oligomeric compounds disclosed herein are in aqueous solution with sodium. In certain embodiments, oligomeric compounds are in aqueous solution with potassium. In certain embodiments, oligomeric compounds are in PBS. In certain embodiments, oligomeric compounds are in water. In certain such embodiments, the pH of the solution is adjusted with NaOH and/or HCl to achieve a desired pH.

VIII. Certain Hotspot Regions

1. Nucleobases 3521-3554 of SEQ ID NO: 2

In certain embodiments, nucleobases 3521-3554 of SEQ ID NO: 2 comprise a hotspot region. In certain embodiments, modified oligonucleotides are complementary to a portion of nucleobases 3521-3554 of SEQ ID NO: 2. In certain embodiments, the modified oligonucleotides are 16 to 20 nucleobases in length. In certain embodiments, the modified oligonucleotides are gapmers. In certain embodiments, the modified oligonucleotides comprise a 2′-MOE nucleoside, a 2′-OMe nucleoside, a cEt nucleoside, or a combination thereof. In certain embodiments, the internucleoside linkages of the modified nucleotides are phosphorothioate internucleoside linkages or phosphodiester linkages, or a combination thereof.

The nucleobase sequences of SEQ ID NOs: 1053, 1129, 2166, 2167, 2168, 2169, 2170, 2171, 2172, 2173, 2174, 2175, 2176, 2242, and 2247 are complementary to nucleobases 3521-3554 of SEQ ID NO: 2.

The nucleobase sequences of Compound Nos: 833560, 833561, 936068, 936108, 936146, 936178, 936218, 936256, 936288, 936290, 936291, 936292, 936293, 936294, 936297, 936298, 936299, 936300, and 936301 are complementary to nucleobases 3521-3554 of SEQ ID NO: 2.

In certain embodiments, modified oligonucleotides complementary to a portion of nucleobases 3521-3554 of SEQ ID NO: 2 achieve at least 27% reduction of SPDEF RNA in a standard cell assay. In certain embodiments, modified oligonucleotides complementary to a portion of nucleobases 3521-3554 of SEQ ID NO: 2 achieve an average of 55% reduction of SPDEF RNA in a standard cell assay.

2. Nucleobases 3684-3702 of SEQ ID NO: 2

In certain embodiments, nucleobases 3684-3702 of SEQ ID NO: 2 comprise a hotspot region. In certain embodiments, modified oligonucleotides are complementary to a portion of nucleobases 3684-3702 of SEQ ID NO: 2. In certain embodiments, the modified oligonucleotides are 16 to 20 nucleobases in length. In certain embodiments, the modified oligonucleotides are gapmers. In certain embodiments, the modified oligonucleotides comprise a 2′-MOE nucleoside, a 2′-OMe nucleoside, a cEt nucleoside, or a combination thereof. In certain embodiments, the internucleoside linkages of the modified nucleotides are phosphorothioate internucleoside linkages or phosphodiester linkages, or a combination thereof.

The nucleobase sequences of SEQ ID NOs: 1777, 1852, 1928, and 2004 are complementary to nucleobases 3684-3702 of SEQ ID NO: 2.

The nucleobase sequences of Compound NOs: 854213, 854214, 854215, 854216, 936069, 936109, 936147, 936179, 936219, and 936257 are complementary to nucleobases 3684-3702 of SEQ ID NO: 2.

In certain embodiments, modified oligonucleotides complementary to a portion of nucleobases 3684-3702 of SEQ ID NO: 2 achieve at least 45% reduction of SPDEF RNA in a standard cell assay. In certain embodiments, modified oligonucleotides complementary to a portion of nucleobases 3684-3702 of SEQ ID NO: 2 achieve an average of 57% reduction of SPDEF RNA in a standard cell assay.

3. Nucleobases 3785-3821 of SEQ ID NO: 2

In certain embodiments, nucleobases 3785-3821 of SEQ ID NO: 2 comprise a hotspot region. In certain embodiments, modified oligonucleotides are complementary to a portion of nucleobases 3785-3821 of SEQ ID NO: 2. In certain embodiments, the modified oligonucleotides are 16 to 20 nucleobases in length. In certain embodiments, the modified oligonucleotides are gapmers. In certain embodiments, the modified oligonucleotides comprise a 2′-MOE nucleoside, a 2′-OMe nucleoside, a cEt nucleoside, or a combination thereof. In certain embodiments, the internucleoside linkages of the modified nucleotides are phosphorothioate internucleoside linkages or phosphodiester linkages, or a combination thereof.

The nucleobase sequences of SEQ ID NOs: 1282, 1358, 1434, 2177, 2178, 2179, 2180, 2181, 2182, 2183, 2184, 2185, and 2186 are complementary to nucleobases 3785-3821 of SEQ ID NO: 2.

The nucleobase sequences of Compound Nos: 833579, 833580, 833581, 936070, 936110, 936148, 936180, 936220, 936258, 936310, 936311, 936312, 936313, 936314, 936315, 936316, 936317, 936318, and 936325 are complementary to nucleobases 3785-3821 of SEQ ID NO: 2.

In certain embodiments, modified oligonucleotides complementary to a portion of nucleobases 3785-3821 of SEQ ID NO: 2 achieve at least 37% reduction of SPDEF RNA in a standard cell assay. In certain embodiments, modified oligonucleotides complementary to a portion of nucleobases 3785-3821 of SEQ ID NO: 2 achieve an average of 60% reduction of SPDEF RNA in a standard cell assay.

4. Nucleobases 6356-6377 of SEQ ID NO: 2

In certain embodiments, nucleobases 6356-6377 of SEQ ID NO: 2 comprise a hotspot region. In certain embodiments, modified oligonucleotides are complementary to a portion of nucleobases 6356-6377 of SEQ ID NO: 2. In certain embodiments, the modified oligonucleotides are 16 to 20 nucleobases in length. In certain embodiments, the modified oligonucleotides are gapmers. In certain embodiments, the modified oligonucleotides comprise a 2′-MOE nucleoside, a 2′-OMe nucleoside, a cEt nucleoside, or a combination thereof. In certain embodiments, the internucleoside linkages of the modified nucleotides are phosphorothioate internucleoside linkages or phosphodiester linkages, or a combination thereof.

The nucleobase sequences of SEQ ID NOs: 678, 2198, 2199, 2200, 2244, and 2248 are complementary to nucleobases 6356-6377 of SEQ ID NO: 2.

The nucleobase sequences of Compound Nos: 833635, 936079, 936119, 936154, 936189, 936229, 936264, 936347, 936348, and 936349 are complementary to nucleobases 6356-6377 of SEQ ID NO: 2.

In certain embodiments, modified oligonucleotides complementary to a portion of nucleobases 6356-6377 of SEQ ID NO: 2 achieve at least 38% reduction of SPDEF RNA in a standard cell assay. In certain embodiments, modified oligonucleotides complementary to a portion of nucleobases 6356-6377 of SEQ ID NO: 2 achieve an average of 53% reduction of SPDEF RNA in a standard cell assay.

5. Nucleobases 8809-8826 of SEQ ID NO: 2

In certain embodiments, nucleobases 8809-8826 of SEQ ID NO: 2 comprise a hotspot region. In certain embodiments, modified oligonucleotides are complementary to a portion of nucleobases 8809-8826 of SEQ ID NO: 2. In certain embodiments, the modified oligonucleotides are 16 to 20 nucleobases in length. In certain embodiments, the modified oligonucleotides are gapmers. In certain embodiments, the modified oligonucleotides comprise a 2′-MOE nucleoside, a 2′-OMe nucleoside, a cEt nucleoside, or a combination thereof. In certain embodiments, the internucleoside linkages of the modified nucleotides are phosphorothioate internucleoside linkages or phosphodiester linkages, or a combination thereof.

The nucleobase sequences of SEQ ID NOs: 683, 1715, and 2245 are complementary to nucleobases 8809-8826 of SEQ ID NO: 2.

The nucleobase sequences of Compound Nos: 833715, 854302, 936081, 936082, 936121, 936191, 936192, and 936231 are complementary to nucleobases 8809-8826 of SEQ ID NO: 2.

In certain embodiments, modified oligonucleotides complementary to a portion of nucleobases 8809-8826 of SEQ ID NO: 2 achieve at least 52% reduction of SPDEF RNA in a standard cell assay. In certain embodiments, modified oligonucleotides complementary to a portion of nucleobases 8809-8826 of SEQ ID NO: 2 achieve an average of 66% reduction of SPDEF RNA in a standard cell assay.

6. Nucleobases 9800-9817 of SEQ ID NO: 2

In certain embodiments, nucleobases 9800-9817 of SEQ ID NO: 2 comprise a hotspot region. In certain embodiments, modified oligonucleotides are complementary to a portion of nucleobases 9800-9817 of SEQ ID NO: 2. In certain embodiments, the modified oligonucleotides are 16 to 20 nucleobases in length. In certain embodiments, the modified oligonucleotides are gapmers. In certain embodiments, the modified oligonucleotides comprise a 2′-MOE nucleoside, a 2′-OMe nucleoside, a cEt nucleoside, or a combination thereof. In certain embodiments, the internucleoside linkages of the modified nucleotides are phosphorothioate internucleoside linkages or phosphodiester linkages, or a combination thereof.

The nucleobase sequences of SEQ ID NOs: 761, 2229, and 2230 are complementary to nucleobases 9800-9817 of SEQ ID NO: 2.

The nucleobase sequences of Compound Nos: 833748, 936084, 936123, 936158, 936194, 936233, 936268, 936409, and 936410 are complementary to nucleobases 9800-9817 of SEQ ID NO: 2.

In certain embodiments, modified oligonucleotides complementary to a portion of nucleobases 9800-9817 of SEQ ID NO: 2 achieve at least 51% reduction of SPDEF RNA in a standard cell assay. In certain embodiments, modified oligonucleotides complementary to a portion of nucleobases 9800-9817 of SEQ ID NO: 2 achieve an average of 58% reduction of SPDEF RNA in a standard cell assay.

7. Nucleobases 14212-14231 of SEQ ID NO: 2

In certain embodiments, nucleobases 14212-14231 of SEQ ID NO: 2 comprise a hotspot region. In certain embodiments, modified oligonucleotides are complementary to a portion of nucleobases 14212-14231 of SEQ ID NO: 2. In certain embodiments, the modified oligonucleotides are 16 to 20 nucleobases in length. In certain embodiments, the modified oligonucleotides are gapmers. In certain embodiments, the modified oligonucleotides comprise a 2′-MOE nucleoside, a 2′-OMe nucleoside, a cEt nucleoside, or a combination thereof. In certain embodiments, the internucleoside linkages of the modified nucleotides are phosphorothioate internucleoside linkages or phosphodiester linkages, or a combination thereof.

The nucleobase sequences of SEQ ID NOs: 1606, 1682, 2255, 2275, and 2280 are complementary to nucleobases 14212-14231 of SEQ ID NO: 2.

The nucleobase sequences of Compound Nos: 833886, 833887, 936096, 936097, 936135, 936136, 936169, 936206, 936207, 936245, 936246, 936279, and 936442 are complementary to nucleobases 14212-14231 of SEQ ID NO: 2.

In certain embodiments, modified oligonucleotides complementary to a portion of nucleobases 14212-14231 of SEQ ID NO: 2 achieve at least 45% reduction of SPDEF RNA in a standard cell assay. In certain embodiments, modified oligonucleotides complementary to a portion of nucleobases 14212-14231 of SEQ ID NO: 2 achieve an average of 59% reduction of SPDEF RNA in a standard cell assay.

8. Nucleobases 15385-15408 of SEQ ID NO: 2

In certain embodiments, nucleobases 15385-15408 of SEQ ID NO: 2 comprise a hotspot region. In certain embodiments, modified oligonucleotides are complementary to a portion of nucleobases 15385-15408 of SEQ ID NO: 2. In certain embodiments, the modified oligonucleotides are 16 to 20 nucleobases in length. In certain embodiments, the modified oligonucleotides are gapmers. In certain embodiments, the modified oligonucleotides comprise a 2′-MOE nucleoside, a 2′-OMe nucleoside, a cEt nucleoside, or a combination thereof. In certain embodiments, the internucleoside linkages of the modified nucleotides are phosphorothioate internucleoside linkages or phosphodiester linkages, or a combination thereof.

The nucleobase sequences of SEQ ID NOs: 999, 1075, 2262, 2263, 2264, 2265, 2266, 2267, and 2268 are complementary to nucleobases 15385-15408 of SEQ ID NO: 2.

The nucleobase sequences of Compound Nos: 833910, 833911, 936098, 936137, 936170, 936208, 936247, 936280, 936452, 936453, 936454, 936455, 936456, 936457, and 936458 are complementary to nucleobases 15385-15408 of SEQ ID NO: 2.

In certain embodiments, modified oligonucleotides complementary to a portion of nucleobases 15385-15408 of SEQ ID NO: 2 achieve at least 44% reduction of SPDEF RNA in a standard cell assay. In certain embodiments, modified oligonucleotides complementary to a portion of nucleobases 15385-15408 of SEQ ID NO: 2 achieve an average of 59% reduction of SPDEF RNA in a standard cell assay.

9. Nucleobases 17289-17307 of SEQ ID NO: 2

In certain embodiments, nucleobases 17289-17307 of SEQ ID NO: 2 comprise a hotspot region. In certain embodiments, modified oligonucleotides are complementary to a portion of nucleobases 17289-17307 of SEQ ID NO: 2. In certain embodiments, the modified oligonucleotides are 16 to 20 nucleobases in length. In certain embodiments, the modified oligonucleotides are gapmers. In certain embodiments, the modified oligonucleotides comprise a 2′-MOE nucleoside, a 2′-OMe nucleoside, a cEt nucleoside, or a combination thereof. In certain embodiments, the internucleoside linkages of the modified nucleotides are phosphorothioate internucleoside linkages or phosphodiester linkages, or a combination thereof.

The nucleobase sequences of SEQ ID NOs: 163, 1980, 2056, and 2277 are complementary to nucleobases 17289-17307 of SEQ ID NO: 2.

The nucleobase sequences of Compound Nos: 802094, 854526, 854527, 936100, 936101, 936139, 936210, 936211, and 936249 are complementary to nucleobases 17289-17307 of SEQ ID NO: 2.

In certain embodiments, modified oligonucleotides complementary to a portion of nucleobases 17289-17307 of SEQ ID NO: 2 achieve at least 43% reduction of SPDEF RNA in a standard cell assay. In certain embodiments, modified oligonucleotides complementary to a portion of nucleobases 17289-17307 of SEQ ID NO: 2 achieve an average of 60% reduction of SPDEF RNA in a standard cell assay.

10. Nucleobases 17490-17509 of SEQ ID NO: 2

In certain embodiments, nucleobases 17490-17509 of SEQ ID NO: 2 comprise a hotspot region. In certain embodiments, modified oligonucleotides are complementary to a portion of nucleobases 17490-17509 of SEQ ID NO: 2. In certain embodiments, the modified oligonucleotides are 16 to 20 nucleobases in length. In certain embodiments, the modified oligonucleotides are gapmers. In certain embodiments, the modified oligonucleotides comprise a 2′-MOE nucleoside, a 2′-OMe nucleoside, a cEt nucleoside, or a combination thereof. In certain embodiments, the internucleoside linkages of the modified nucleotides are phosphorothioate internucleoside linkages or phosphodiester linkages, or a combination thereof.

The nucleobase sequences of SEQ ID NOs: 1831, 1907, 1983, 2059, and 2282 are complementary to nucleobases 17490-17509 of SEQ ID NO: 2.

The nucleobase sequences of Compound Nos: 854542, 854543, 854544, 854545, 936104, 936142, 936174, 936214, 936252, and 936284 are complementary to nucleobases 17490-17509 of SEQ ID NO: 2.

In certain embodiments, modified oligonucleotides complementary to a portion of nucleobases 17490-17509 of SEQ ID NO: 2 achieve at least 39% reduction of SPDEF RNA in a standard cell assay. In certain embodiments, modified oligonucleotides complementary to a portion of nucleobases 17490-17509 of SEQ ID NO: 2 achieve an average of 63% reduction of SPDEF RNA in a standard cell assay.

11. Nucleobases 19600-19642 of SEQ ID NO: 2

In certain embodiments, nucleobases 19600-19642 of SEQ ID NO: 2 comprise a hotspot region. In certain embodiments, oligomeric compounds or oligomeric duplexes comprise modified oligonucleotides that are complementary within nucleobases 19600-19642 of SEQ ID NO: 2. In certain embodiments, modified oligonucleotides are 23 nucleobases in length. In certain embodiments, modified oligonucleotides are antisense RNAi oligonucleotides. In certain embodiments, the antisense RNAi oligonucleotide has a sugar motif (from 5′ to 3′) of: yfyfyfyfyfyfyfyfyfyfyyy; wherein “y” represents a 2′-O-methylribosyl sugar, and the “f” represents a 2′-fluororibosyl sugar; and a linkage motif (from 5′ to 3′) of: ssooooooooooooooooooss; wherein ‘o’ represents a phosphodiester internucleoside linkage and 's′ represents a phosphorothioate internucleoside linkage.

The nucleobase sequences of SEQ ID NOs: 2670, 2582, and 2677 are complementary within nucleobases 19600-19642 of SEQ ID NO: 2.

RNAi compounds 1537312, 1527655, and 1537332 comprise an antisense RNAi oligonucleotide that is complementary within nucleobases 19600-19642 of SEQ ID NO: 2.

In certain embodiments, modified oligonucleotides complementary within nucleobases 19600-19642 of SEQ ID NO: 2 achieve at least 59% reduction of SPDEF RNA in a standard cell assay. In certain embodiments, modified oligonucleotides complementary within nucleobases 19600-19642 of SEQ ID NO: 2 achieve an average of 67% reduction of SPDEF RNA in a standard cell assay.

12. Nucleobases 19640-19672 of SEQ ID NO: 2

In certain embodiments, nucleobases 19640-19672 of SEQ ID NO: 2 comprise a hotspot region. In certain embodiments, oligomeric compounds or oligomeric duplexes comprise modified oligonucleotides that are complementary within nucleobases 19640-19672 of SEQ ID NO: 2. In certain embodiments, modified oligonucleotides are 23 nucleobases in length. In certain embodiments, modified oligonucleotides are antisense RNAi oligonucleotides. In certain embodiments, the antisense RNAi oligonucleotide has a sugar motif (from 5′ to 3′) of: yfyfyfyfyfyfyfyfyfyfyyy; wherein “y” represents a 2′-O-methylribosyl sugar, and the “f” represents a 2′-fluororibosyl sugar; and a linkage motif (from 5′ to 3′) of: ssooooooooooooooooooss; wherein ‘o’ represents a phosphodiester internucleoside linkage and 's′ represents a phosphorothioate internucleoside linkage.

The nucleobase sequences of SEQ ID NOs: 2609, 2606, and 2578 are complementary within nucleobases 19640-19672 of SEQ ID NO: 2.

RNAi compounds 1528397, 1528231, and 1527651 comprise an antisense RNAi oligonucleotide that is complementary within nucleobases 19640-19672 of SEQ ID NO: 2.

In certain embodiments, modified oligonucleotides complementary within nucleobases 19640-19672 of SEQ ID NO: 2 achieve at least 33% reduction of SPDEF RNA in a standard cell assay. In certain embodiments, modified oligonucleotides complementary within nucleobases 19640-19672 of SEQ ID NO: 2 achieve an average of 59% reduction of SPDEF RNA in a standard cell assay.

### Nonlimiting Disclosure and Incorporation by Reference

Each of the literature and patent publications listed herein is incorporated by reference in its entirety.

While certain compounds, compositions and methods described herein have been described with specificity in accordance with certain embodiments, the following examples serve only to illustrate the compounds described herein and are not intended to limit the same. Each of the references, GenBank accession numbers, and the like recited in the present application is incorporated herein by reference in its entirety.

Although the sequence listing accompanying this filing identifies each sequence as either “RNA” or “DNA” as required, in reality, those sequences may be modified with any combination of chemical modifications. One of skill in the art will readily appreciate that such designation as “RNA” or “DNA” to describe modified oligonucleotides is, in certain instances, arbitrary. For example, an oligonucleotide comprising a nucleoside comprising a 2′-OH sugar moiety and a thymine base could be described as a DNA having a modified sugar (2′-OH in place of one 2′-H of DNA) or as an RNA having a modified base (thymine (methylated uracil) in place of a uracil of RNA). Accordingly, nucleic acid sequences provided herein, including, but not limited to those in the sequence listing, are intended to encompass nucleic acids containing any combination of natural or modified RNA and/or DNA, including, but not limited to such nucleic acids having modified nucleobases. By way of further example and without limitation, an oligomeric compound having the nucleobase sequence “ATCGATCG” encompasses any oligomeric compounds having such nucleobase sequence, whether modified or unmodified, including, but not limited to, such compounds comprising RNA bases, such as those having sequence “AUCGAUCG” and those having some DNA bases and some RNA bases such as “AUCGATCG” and oligomeric compounds having other modified nucleobases, such as “AT^{m}CGAUCG,” wherein mC indicates a cytosine base comprising a methyl group at the 5-position.

Certain compounds described herein (e.g., modified oligonucleotides) have one or more asymmetric center and thus give rise to enantiomers, diastereomers, and other stereoisomeric configurations that may be defined, in terms of absolute stereochemistry, as (R) or (S), as a or such as for sugar anomers, or as (D) or (L), such as for amino acids, etc. Compounds provided herein that are drawn or described as having certain stereoisomeric configurations include only the indicated compounds. Compounds provided herein that are drawn or described with undefined stereochemistry include all such possible isomers, including their stereorandom and optically pure forms, unless specified otherwise. Likewise, tautomeric forms of the compounds herein are also included unless otherwise indicated. Unless otherwise indicated, compounds described herein are intended to include corresponding salt forms.

The compounds described herein include variations in which one or more atoms are replaced with a non-radioactive isotope or radioactive isotope of the indicated element. For example, compounds herein that comprise hydrogen atoms encompass all possible deuterium substitutions for each of the ¹H hydrogen atoms. Isotopic substitutions encompassed by the compounds herein include but are not limited to: ²H or ³H in place of ¹H, ¹³C or ¹⁴C in place of ¹²C, ¹⁵N in place of ¹⁴N, ¹⁷O or ¹⁸O in place of ¹⁶O, and ³³S, ³⁴S, ³⁵S, or ³⁶S in place of ³²S. In certain embodiments, non-radioactive isotopic substitutions may impart new properties on the oligomeric compound that are beneficial for use as a therapeutic or research tool. In certain embodiments, radioactive isotopic substitutions may make the compound suitable for research or diagnostic purposes such as imaging.

### EXAMPLES

The following examples illustrate certain embodiments of the present disclosure and are not limiting. Moreover, where specific embodiments are provided, the inventors have contemplated generic application of those specific embodiments. For example, disclosure of an oligonucleotide having a particular motif provides reasonable support for additional oligonucleotides having the same or similar motif And, for example, where a particular high-affinity modification appears at a particular position, other high-affinity modifications at the same position are considered suitable, unless otherwise indicated.

### Example 1: Effect of 3-10-3 cEt Gapmer Modified Oligonucleotides on Human SPDEF RNA In Vitro, Single Dose

Modified oligonucleotides complementary to human SPDEF nucleic acid were tested for their effect on SPDEF RNA levels in vitro.

The newly designed modified oligonucleotides in the tables below were designed as 3-10-3 cEt gapmers. The gapmers are 16 nucleosides in length, wherein the central gap segment comprises of ten 2′-deoxynucleosides and is flanked by wing segments on the 5′ direction and the 3′ direction comprising three nucleosides each. Each nucleoside in the 5′ wing segment and each nucleoside in the 3′ wing segment has a cEt sugar modification. The internucleoside linkages throughout each gapmer are phosphorothioate (P═S) linkages. All cytosine residues throughout each gapmer are 5-methylcytosines.

“Start site” indicates the 5′-most nucleoside to which the modified oligonucleotide is complementary in the human gene sequence. “Stop site” indicates the 3′-most nucleoside to which the modified oligonucleotide is complementary in the human gene sequence. Each modified oligonucleotide listed in the Tables below is 100% complementary to SEQ ID NO: 1 (GENBANK Accession No. NM_012391.2), SEQ ID NO: 2 (the complement of GENBANK Accession No. NC_000006.12 truncated from nucleotides 34536001 to 34558000), SEQ ID NO: 3 (GENBANK Accession No. NM_001252294.1), SEQ ID NO: 4 (GENBANK Accession No. XM_005248988.3) or SEQ ID NO: 5 (GENBANK Accession No. XM 006715048.1). ‘N/A’ indicates that the modified oligonucleotide is not 100% complementary to that particular gene sequence.

Cultured VCaP cells at a density of 20,000 cells per well were treated with 4 μM modified oligonucleotide by electroporation. After a treatment period of approximately 24 hours, total RNA was isolated from the cells and SPDEF RNA levels were measured by quantitative real-time RTPCR. Human SPDEF primer probe set RTS35007 (forward sequence CGCTTCATTAGGTGGCTCAA, designated herein as SEQ ID NO: 6; reverse sequence GCTCAGCTTGTCGTAGTTCA, designated herein as SEQ ID NO: 7; probe sequence AATTGAGGACTCAGCCCAGGTGG, designated herein as SEQ ID NO: 8) was used to measure RNA levels. SPDEF RNA levels were normalized to total RNA content, as measured by RIBOGREEN®. Reduction of SPDEF RNA is presented in the tables below as percent SPDEF RNA levels relative to untreated control (UTC) cells. Each table represents results from an individual assay plate. The modified oligonucleotides marked with an asterisk (*) indicate that the modified oligonucleotide is complementary to the amplicon region of the primer probe set. Additional assays may be used to measure the potency and efficacy of the modified oligonucleotides complementary to the amplicon region.

TABLE 1
Reduction of SPDEF RNA by 4 μM 3-10-3 cEt gapmers with a
phosphorothioate backbone

| | SEQ | SEQ | SEQ | SEQ | | | |
|---|---|---|---|---|---|---|---|
| | ID | ID | ID | ID | | | |
| | NO: 1 | NO: 1 | NO: 2 | NO: 2 | | SPDEF | |
| Compound | Start | Stop | Start | Stop | | (% | SEQ |
| Number | Site | Site | Site | Site | Sequence (5′ to 3′) | UTC) | ID NO |
| 652519* | 1353 | 1368 | 19638 | 19653 | ACTGGCGGATGGAGCG | 123 | 15 |
| 652522 | 1362 | 1377 | 19647 | 19662 | TCTTGTAATACTGGCG | 57 | 16 |
| 652635 | N/A | N/A | 8818 | 8833 | ACATGTCTGGATTAAG | 44 | 17 |
| 801677 | 12 | 27 | 1679 | 1694 | AGTCAGACAGCCGCGA | 68 | 18 |
| 801682 | 46 | 61 | 1713 | 1728 | GGACACGGCAGAGTGC | 75 | 19 |
| 801688 | 75 | 90 | 1742 | 1757 | CTTGGAGGACTGGGTC | 96 | 20 |
| 801694 | 124 | 139 | 1791 | 1806 | CACCGTGGCAAGGCCC | 68 | 21 |
| 801700 | 167 | 182 | 1834 | 1849 | CCTGTAGGGAGTCCCC | 62 | 22 |
| 801706 | 278 | 293 | 1945 | 1960 | GCCAGCGGAACCAGGG | 56 | 23 |
| 801712 | 390 | 405 | 2057 | 2072 | CTGTTAGCTGCCTGGT | 66 | 24 |
| 801718 | 417 | 432 | 13528 | 13543 | CGCTGCTGTTTGGGCT | 95 | 25 |
| 801724 | 450 | 465 | 13561 | 13576 | CGCTGCTCAGACCCGG | 66 | 26 |
| 801730 | 500 | 515 | 13611 | 13626 | GCCTGTCCGCGACACC | 45 | 27 |
| 801736 | 542 | 557 | 13653 | 13668 | CCGTCTCTCGAGACCC | 55 | 28 |
| 801742 | 562 | 577 | 13673 | 13688 | GGTGGACTGGGACTCC | 81 | 29 |
| 801748 | 599 | 614 | 13710 | 13725 | GAGGTAGAAGGCGGAC | 78 | 30 |
| 801754 | 624 | 639 | 13735 | 13750 | CAGGGTACAGCATGTC | 58 | 31 |
| 801760 | 678 | 693 | 13789 | 13804 | GTGGCTCCTCCCGACT | 61 | 32 |
| 801766 | 712 | 727 | 13823 | 13838 | CTGTCAATGACCGGGC | 37 | 33 |
| 801772 | 755 | 770 | 13866 | 13881 | CAGCCCGCCGGGCACC | 62 | 34 |
| 801778 | 779 | 794 | 13890 | 13905 | CTCCAGCGAGTGCTCC | 105 | 35 |
| 801784 | 807 | 822 | 13918 | 13933 | CTTCGCCCACCACCAT | 90 | 36 |
| 801790 | 828 | 843 | 13939 | 13954 | CCGTCTCGATGTCCTT | 51 | 37 |
| 801795 | 854 | 869 | 13965 | 13980 | TGCGGTGATGTTGAGC | 93 | 38 |
| 801801 | 873 | 888 | 16822 | 16837 | GGCTCCAGTCCATGGG | 87 | 39 |
| 801807 | 926 | 941 | 16875 | 16890 | GGGCAGCCGGTATTGG | 112 | 40 |
| 801813 | 988 | 1003 | 16937 | 16952 | TGCTCCTCCGACATGG | 69 | 41 |
| 801819 | 1052 | 1067 | 17001 | 17016 | TGACTTCCAGATGTCC | 101 | 42 |
| 801823 | 1081 | 1096 | 18451 | 18466 | GGTGAAGTCCGCTCTT | 83 | 43 |
| 801829 | 1100 | 1115 | 18470 | 18485 | ACAGTAGTGAATCGCC | 70 | 44 |
| 801835 | 1130 | 1145 | 18618 | 18633 | GTCGGTCCAGCTCTCC | 88 | 45 |
| 801841 | 1151 | 1166 | 18639 | 18654 | GCATGATGAGTCCACC | 92 | 46 |
| 801847 | 1170 | 1185 | 18658 | 18673 | GGTGGATGGGCTGCCC | 54 | 47 |
| 801853 | 1202 | 1217 | 18690 | 18705 | CTTGAGTAGCAACTCC | 51 | 48 |
| 801856* | 1231 | 1246 | 18719 | 18734 | CACCTAATGAAGCGGC | 45 | 49 |
| 801862* | 1274 | 1289 | 19559 | 19574 | GGCTGAGTCCTCAATT | 53 | 50 |
| 801868* | 1311 | 1326 | 19596 | 19611 | GACGGTTCTTGCGGAT | 12 | 51 |
| 801874* | 1340 | 1355 | 19625 | 19640 | GCGGCTCAGCTTGTCG | 29 | 52 |
| 801879 | 1370 | 1385 | 19655 | 19670 | GATGCCCTTCTTGTAA | 63 | 53 |
| 801885 | 1393 | 1408 | 19678 | 19693 | TGGGAGATGTCTGGCT | 98 | 54 |
| 801891 | 1420 | 1435 | 19705 | 19720 | GGGTGCACGAACTGGT | 57 | 55 |
| 801897 | 1577 | 1592 | 19862 | 19877 | GGCAGTTGGTTGCCCC | 61 | 56 |
| 801903 | 1616 | 1631 | 19901 | 19916 | CAGGGTCCCGAAGGCC | 85 | 57 |
| 801909 | 1746 | 1761 | 20031 | 20046 | GTCGAGTCACTGCCCT | 36 | 58 |
| 801915 | 1810 | 1825 | 20095 | 20110 | GTGTGGTGCAGAATGG | 94 | 59 |
| 801927 | N/A | N/A | 5131 | 5146 | CAGAGACACATCCCCC | 53 | 60 |
| 801933 | N/A | N/A | 2358 | 2373 | GCACGGCGGCCTCCCC | 44 | 61 |
| 801939 | N/A | N/A | 2825 | 2840 | GGGCACCCAGTCGCCC | 81 | 62 |
| 801945 | N/A | N/A | 3317 | 3332 | GGGTCCTTGGCTCTGG | 67 | 63 |
| 801951 | N/A | N/A | 3825 | 3840 | GTCCCCCTGTCAGACT | 65 | 64 |
| 801957 | N/A | N/A | 4235 | 4250 | GGGCGAGAGAGTGGAG | 86 | 65 |
| 801963 | N/A | N/A | 4916 | 4931 | ATCCTGGTGGTGCGCC | 86 | 66 |
| 801969 | N/A | N/A | 5446 | 5461 | TGCGGCCCCTCCAGAC | 70 | 67 |
| 801975 | N/A | N/A | 5818 | 5833 | TGAAGGGCCGGCCACA | 65 | 68 |
| 801981 | N/A | N/A | 6181 | 6196 | CAGTGCCTCCCCGCCT | 52 | 69 |
| 801987 | N/A | N/A | 6549 | 6564 | GGTGAGTCCCTGGTCC | 75 | 70 |
| 801993 | N/A | N/A | 7033 | 7048 | GCACTACTTCCAGCGC | 89 | 71 |
| 801999 | N/A | N/A | 7406 | 7421 | TCTCCGGGCTTTCCCC | 43 | 72 |
| 802005 | N/A | N/A | 7920 | 7935 | GGGCTACCCAGGCCTC | 90 | 73 |
| 802011 | N/A | N/A | 8293 | 8308 | ATGTATCCTCACCCCT | 63 | 74 |
| 802022 | N/A | N/A | 9208 | 9223 | CCCCAGCGAGCCCTCC | 61 | 75 |
| 802028 | N/A | N/A | 9790 | 9805 | GCGGACAGTGAGGCTC | 55 | 76 |
| 802034 | N/A | N/A | 10241 | 10256 | GACTCCTGGCTCGGGC | 74 | 77 |
| 802040 | N/A | N/A | 10829 | 10844 | CCCTTGTGGCCCTCCT | 60 | 78 |
| 802045 | N/A | N/A | 11422 | 11437 | TCCCCCTGGATAGCAT | 56 | 79 |
| 802051 | N/A | N/A | 12032 | 12047 | CGTCAAGCCAGAGGCA | 43 | 80 |
| 802057 | N/A | N/A | 12815 | 12830 | TGGTACCCACCTCCCC | 65 | 81 |
| 802063 | N/A | N/A | 13512 | 13527 | GGCGGCTGTGTCTACG | 80 | 82 |
| 802069 | N/A | N/A | 14448 | 14463 | GCTCATGGGCAGCAAT | 65 | 83 |
| 802075 | N/A | N/A | 15727 | 15742 | CTGCAATGCCAGGGCC | 51 | 84 |
| 802081 | N/A | N/A | 16172 | 16187 | GCCCTTGGCTAGGTCC | 61 | 85 |
| 802087 | N/A | N/A | 16666 | 16681 | GGGCCCCTGTGGAAGT | 88 | 86 |
| 802093 | N/A | N/A | 17204 | 17219 | GGCCTTGACCAGGGCT | 77 | 87 |
| 802099 | N/A | N/A | 18204 | 18219 | GGCTTGCATGCAACCC | 62 | 88 |
| 802105 | N/A | N/A | 18596 | 18611 | GTCGAGGCTGGGTGGC | 109 | 89 |
| 802111* | N/A | N/A | 19067 | 19082 | ATTCTCAGGCAGTTCG | 52 | 90 |
| 802117* | N/A | N/A | 19522 | 19537 | GGGCCCCGAGAGAGCC | 105 | 91 |

TABLE 2
Reduction of SPDEF RNA by 4 μM 3-10-3 cEt gapmers with a
phosphorothioate backbone

| | SEQ | SEQ | SEQ | SEQ | | | |
|---|---|---|---|---|---|---|---|
| | ID | ID | ID | ID | | | |
| | NO: 1 | NO: 1 | NO: 2 | NO: 2 | | SPDEF | |
| Compound | Start | Stop | Start | Stop | | (% | SEQ |
| Number | Site | Site | Site | Site | Sequence (5′ to 3′) | UTC) | ID NO |
| 652365 | 33 | 48 | 1700 | 1715 | TGCAGGAATGTGCTGG | 81 | 92 |
| 652522 | 1362 | 1377 | 19647 | 19662 | TCTTGTAATACTGGCG | 73 | 16 |
| 652649 | N/A | N/A | 10940 | 10955 | CCCGTCCACATCCCCA | 68 | 93 |
| 791840 | 1056 | 1071 | N/A | N/A | CCGCTGACTTCCAGAT | 84 | 94 |
| 791899 | 1355 | 1370 | 19640 | 19655 | ATACTGGCGGATGGAG | 103 | 95 |
| 801683 | 48 | 63 | 1715 | 1730 | GTGGACACGGCAGAGT | 59 | 96 |
| 801689 | 77 | 92 | 1744 | 1759 | GGCTTGGAGGACTGGG | 78 | 97 |
| 801695 | 126 | 141 | 1793 | 1808 | GGCACCGTGGCAAGGC | 105 | 98 |
| 801701 | 171 | 186 | 1838 | 1853 | CGTGCCTGTAGGGAGT | 89 | 99 |
| 801707 | 280 | 295 | 1947 | 1962 | GGGCCAGCGGAACCAG | 94 | 100 |
| 801713 | 398 | 413 | N/A | N/A | GGCTGTGTCTGTTAGC | 92 | 101 |
| 801719 | 420 | 435 | 13531 | 13546 | TGCCGCTGCTGTTTGG | 79 | 102 |
| 801725 | 453 | 468 | 13564 | 13579 | ATACGCTGCTCAGACC | 82 | 103 |
| 801731 | 502 | 517 | 13613 | 13628 | AAGCCTGTCCGCGACA | 65 | 104 |
| 801737 | 545 | 560 | 13656 | 13671 | GTCCCGTCTCTCGAGA | 76 | 105 |
| 801743 | 569 | 584 | 13680 | 13695 | CGTGGCGGGTGGACTG | 88 | 106 |
| 801749 | 602 | 617 | 13713 | 13728 | GGAGAGGTAGAAGGCG | 86 | 107 |
| 801755 | 627 | 642 | 13738 | 13753 | CCTCAGGGTACAGCAT | 91 | 108 |
| 801761 | 684 | 699 | 13795 | 13810 | CCTCAGGTGGCTCCTC | 87 | 109 |
| 801767 | 714 | 729 | 13825 | 13840 | GGCTGTCAATGACCGG | 70 | 110 |
| 801773 | 758 | 773 | 13869 | 13884 | GGTCAGCCCGCCGGGC | 76 | 111 |
| 801779 | 782 | 797 | 13893 | 13908 | CTGCTCCAGCGAGTGC | 81 | 112 |
| 801785 | 812 | 827 | 13923 | 13938 | GAGCACTTCGCCCACC | 60 | 113 |
| 801791 | 830 | 845 | 13941 | 13956 | GGCCGTCTCGATGTCC | 123 | 114 |
| 801796 | 857 | 872 | N/A | N/A | ATCTGCGGTGATGTTG | 92 | 115 |
| 801802 | 907 | 922 | 16856 | 16871 | TCTGTCCACAGGAGCC | 72 | 116 |
| 801808 | 965 | 980 | 16914 | 16929 | CTCCTTGCCCGCCAGC | 58 | 117 |
| 801814 | 991 | 1006 | 16940 | 16955 | AACTGCTCCTCCGACA | 87 | 118 |
| 801824 | 1083 | 1098 | 18453 | 18468 | CAGGTGAAGTCCGCTC | 79 | 119 |
| 801830 | 1103 | 1118 | N/A | N/A | GGCACAGTAGTGAATC | 107 | 120 |
| 801836 | 1134 | 1149 | 18622 | 18637 | CGCTGTCGGTCCAGCT | 94 | 121 |
| 801842 | 1154 | 1169 | 18642 | 18657 | GGAGCATGATGAGTCC | 95 | 122 |
| 801848 | 1175 | 1190 | 18663 | 18678 | CCACAGGTGGATGGGC | 99 | 123 |
| 801854 | 1204 | 1219 | 18692 | 18707 | GGCTTGAGTAGCAACT | 68 | 124 |
| 801857* | 1233 | 1248 | 18721 | 18736 | GCCACCTAATGAAGCG | 56 | 125 |
| 801863* | 1294 | 1309 | 19579 | 19594 | CCCCACAGCCGGGCCA | 85 | 126 |
| 801869* | 1313 | 1328 | 19598 | 19613 | GGGACGGTTCTTGCGG | 12 | 127 |
| 801875* | 1341 | 1356 | 19626 | 19641 | AGCGGCTCAGCTTGTC | 24 | 128 |
| 801880 | 1373 | 1388 | 19658 | 19673 | GATGATGCCCTTCTTG | 96 | 129 |
| 801886 | 1397 | 1412 | 19682 | 19697 | GCGCTGGGAGATGTCT | 93 | 130 |
| 801892 | 1455 | 1470 | 19740 | 19755 | GGCGGGTTTCAGGCCC | 86 | 131 |
| 801898 | 1592 | 1607 | 19877 | 19892 | CCCATATCCCCCTGGG | 85 | 132 |
| 801904 | 1620 | 1635 | 19905 | 19920 | GCCCCAGGGTCCCGAA | 62 | 133 |
| 801910 | 1753 | 1768 | 20038 | 20053 | GGCCTTTGTCGAGTCA | 76 | 134 |
| 801916 | 1836 | 1851 | 20121 | 20136 | GCAGATGTCTCCCTGC | 78 | 135 |
| 801928 | N/A | N/A | 5143 | 5158 | GTGAAGTGTCAGCAGA | 62 | 136 |
| 801934 | N/A | N/A | 2444 | 2459 | AGCTGGGTTGGCAGCA | 86 | 137 |
| 801940 | N/A | N/A | 2904 | 2919 | CGCACGCGCACATGCA | 86 | 138 |
| 801946 | N/A | N/A | 3374 | 3389 | CCGAGAATGCCCCCCA | 50 | 139 |
| 801952 | N/A | N/A | 3891 | 3906 | CCCGCCCACGGTCCCA | 86 | 140 |
| 801958 | N/A | N/A | 4485 | 4500 | AGTGACTCAGCCCCCT | 50 | 141 |
| 801964 | N/A | N/A | 4993 | 5008 | TGGAGCCCCGGGCTGG | 80 | 142 |
| 801970 | N/A | N/A | 5503 | 5518 | GTCTCCCGAGAGGTGT | 86 | 143 |
| 801976 | N/A | N/A | 5887 | 5902 | GCCCGGGTCACATGGC | 100 | 144 |
| 801982 | N/A | N/A | 6230 | 6245 | GTCCTGCACCTCACCA | 62 | 145 |
| 801988 | N/A | N/A | 6595 | 6610 | GGTGCAGGTGACACCC | 100 | 146 |
| 801994 | N/A | N/A | 7124 | 7139 | TCCCACGGGCAGCAGG | 82 | 147 |
| 802000 | N/A | N/A | 7615 | 7630 | GACCACCCCGCTGCCC | 95 | 148 |
| 802006 | N/A | N/A | 8000 | 8015 | GGCCAGGTCTTGGCCA | 95 | 149 |
| 802012 | N/A | N/A | 8343 | 8358 | GGTCCCGGCTCTCAGG | 93 | 150 |
| 802017 | N/A | N/A | 8892 | 8907 | GTCCCACGGGCTGCCG | 79 | 151 |
| 802023 | N/A | N/A | 9290 | 9305 | TGCCCCTGTGCTGTGG | 65 | 152 |
| 802029 | N/A | N/A | 9877 | 9892 | AGTGCCACGCCCAGGC | 48 | 153 |
| 802035 | N/A | N/A | 10323 | 10338 | GGCCCAGGTCTTATTC | 73 | 154 |
| 802046 | N/A | N/A | 11472 | 11487 | GGCCACAGCTAGCCCA | 83 | 155 |
| 802052 | N/A | N/A | 12207 | 12222 | GGGTGCCTGATTCTCC | 62 | 156 |
| 802058 | N/A | N/A | 12920 | 12935 | TCCCACAGGGCTATCT | 111 | 157 |
| 802064 | N/A | N/A | 13970 | 13985 | TCACCTGCGGTGATGT | 84 | 158 |
| 802070 | N/A | N/A | 14586 | 14601 | GATATGGTGCGGCACG | 93 | 159 |
| 802076 | N/A | N/A | 15785 | 15800 | GGCCTGAGGGATGCAT | 77 | 160 |
| 802082 | N/A | N/A | 16248 | 16263 | ACATGTGTTGAATAAG | 81 | 161 |
| 802088 | N/A | N/A | 16735 | 16750 | TGGGAACCTGTGGCCT | 79 | 162 |
| 802094 | N/A | N/A | 17292 | 17307 | CACGGTTGTCCCCAGC | 38 | 163 |
| 802100 | N/A | N/A | 18279 | 18294 | GGGAGGCAAGCTGGTT | 93 | 164 |
| 802106* | N/A | N/A | 18759 | 18774 | CGCCCCTTGGGCACCC | 78 | 165 |
| 802112* | N/A | N/A | 19068 | 19083 | TATTCTCAGGCAGTTC | 48 | 166 |
| 802118 | N/A | N/A | 20192 | 20207 | GGGACATGTCAGTTCT | 87 | 167 |

TABLE 3
Reduction of SPDEF RNA by 4 μM 3-10-3 cEt gapmers with a
phosphorothioate backbone

| | SEQ | SEQ | SEQ | SEQ | | | |
|---|---|---|---|---|---|---|---|
| | ID | ID | ID | ID | | | |
| | NO: 1 | NO: 1 | NO: 2 | NO: 2 | | | |
| Compound | Start | Stop | Start | Stop | | SPDEF (% | SEQ |
| Number | Site | Site | Site | Site | Sequence (5′ to 3′) | UTC) | ID NO |
| 652481 | 1063 | 1078 | N/A | N/A | ATCCAGGCCGCTGACT | 94 | 168 |
| 652522 | 1362 | 1377 | 19647 | 19662 | TCTTGTAATACTGGCG | 51 | 16 |
| 791875* | 1220 | 1235 | 18708 | 18723 | GCGGCCATAGCTGTGG | 87 | 169 |
| 791900 | 1357 | 1372 | 19642 | 19657 | TAATACTGGCGGATGG | 89 | 170 |
| 801673 | 2 | 17 | 1669 | 1684 | CCGCGAGATGAAGAGT | 103 | 171 |
| 801678 | 36 | 51 | 1703 | 1718 | GAGTGCAGGAATGTGC | 62 | 172 |
| 801684 | 52 | 67 | 1719 | 1734 | CAGTGTGGACACGGCA | 58 | 173 |
| 801690 | 82 | 97 | 1749 | 1764 | CAGCAGGCTTGGAGGA | 48 | 174 |
| 801696 | 130 | 145 | 1797 | 1812 | TGCTGGCACCGTGGCA | 121 | 175 |
| 801702 | 233 | 248 | 1900 | 1915 | TCAGGTTGGCCACTGG | 93 | 176 |
| 801708 | 337 | 352 | 2004 | 2019 | GCCGGGAAGAGGTGTG | 72 | 177 |
| 801714 | 401 | 416 | N/A | N/A | GGCGGCTGTGTCTGTT | 120 | 178 |
| 801720 | 422 | 437 | 13533 | 13548 | CATGCCGCTGCTGTTT | 83 | 179 |
| 801726 | 456 | 471 | 13567 | 13582 | GGGATACGCTGCTCAG | 61 | 180 |
| 801732 | 506 | 521 | 13617 | 13632 | CTCCAAGCCTGTCCGC | 71 | 181 |
| 801738 | 550 | 565 | 13661 | 13676 | CTCCAGTCCCGTCTCT | 70 | 182 |
| 801744 | 584 | 599 | 13695 | 13710 | CAGGCCCTGCTCGGGC | 100 | 183 |
| 801750 | 606 | 621 | 13717 | 13732 | AGTAGGAGAGGTAGAA | 80 | 184 |
| 801756 | 629 | 644 | 13740 | 13755 | GTCCTCAGGGTACAGC | 56 | 185 |
| 801762 | 693 | 708 | 13804 | 13819 | GCTCAGGCTCCTCAGG | 80 | 186 |
| 801768 | 719 | 734 | 13830 | 13845 | GGCTTGGCTGTCAATG | 75 | 187 |
| 801774 | 761 | 776 | 13872 | 13887 | CAAGGTCAGCCCGCCG | 64 | 188 |
| 801780 | 784 | 799 | 13895 | 13910 | ACCTGCTCCAGCGAGT | 62 | 189 |
| 801786 | 815 | 830 | 13926 | 13941 | CTTGAGCACTTCGCCC | 61 | 190 |
| 801792 | 833 | 848 | 13944 | 13959 | GCAGGCCGTCTCGATG | 102 | 191 |
| 801797 | 860 | 875 | N/A | N/A | GGGATCTGCGGTGATG | 85 | 192 |
| 801803 | 916 | 931 | 16865 | 16880 | TATTGGTGCTCTGTCC | 49 | 193 |
| 801809 | 967 | 982 | 16916 | 16931 | AGCTCCTTGCCCGCCA | 80 | 194 |
| 801815 | 995 | 1010 | 16944 | 16959 | GCGGAACTGCTCCTCC | 73 | 195 |
| 801825 | 1087 | 1102 | 18457 | 18472 | GCCCCAGGTGAAGTCC | 95 | 196 |
| 801831 | 1106 | 1121 | N/A | N/A | CGAGGCACAGTAGTGA | 92 | 197 |
| 801837 | 1138 | 1153 | 18626 | 18641 | ACCTCGCTGTCGGTCC | 73 | 198 |
| 801843 | 1156 | 1171 | 18644 | 18659 | CCGGAGCATGATGAGT | 90 | 199 |
| 801849 | 1177 | 1192 | 18665 | 18680 | TGCCACAGGTGGATGG | 73 | 200 |
| 801858* | 1238 | 1253 | 18726 | 18741 | GTTGAGCCACCTAATG | 27 | 201 |
| 801864* | 1296 | 1311 | 19581 | 19596 | TGCCCCACAGCCGGGC | 73 | 202 |
| 801870* | 1315 | 1330 | 19600 | 19615 | GCGGGACGGTTCTTGC | 19 | 203 |
| 801876* | 1343 | 1358 | 19628 | 19643 | GGAGCGGCTCAGCTTG | 55 | 204 |
| 801881 | 1376 | 1391 | 19661 | 19676 | CCGGATGATGCCCTTC | 54 | 205 |
| 801887 | 1409 | 1424 | 19694 | 19709 | CTGGTAGACGAGGCGC | 88 | 206 |
| 801893 | 1518 | 1533 | 19803 | 19818 | TGCCCGTTTTCCCCCA | 63 | 207 |
| 801899 | 1597 | 1612 | 19882 | 19897 | GAGGACCCATATCCCC | 63 | 208 |
| 801905 | 1634 | 1649 | 19919 | 19934 | GAGGAAGCACCCCTGC | 76 | 209 |
| 801911 | 1755 | 1770 | 20040 | 20055 | GTGGCCTTTGTCGAGT | 58 | 210 |
| 801917 | 1838 | 1853 | 20123 | 20138 | GTGCAGATGTCTCCCT | 48 | 211 |
| 801929 | N/A | N/A | 5145 | 5160 | CTGTGAAGTGTCAGCA | 50 | 212 |
| 801935 | N/A | N/A | 2490 | 2505 | GCCCACTGGTGGCCTG | 102 | 213 |
| 801941 | N/A | N/A | 2997 | 3012 | GCTGGGCGGCCCCAGC | 96 | 214 |
| 801947 | N/A | N/A | 3430 | 3445 | GGGTCCCCTACGCAGT | 67 | 215 |
| 801953 | N/A | N/A | 3978 | 3993 | CCTCCGTGAAGCCTGC | 51 | 216 |
| 801959 | N/A | N/A | 4606 | 4621 | GCCACTCGCTTGGCTG | 80 | 217 |
| 801965 | N/A | N/A | 5080 | 5095 | GGAGCTAGGTCCCAGC | 30 | 218 |
| 801971 | N/A | N/A | 5624 | 5639 | GCCCCTTGGCCGATCC | 64 | 219 |
| 801977 | N/A | N/A | 5965 | 5980 | TGCCCCCGTCAGGCCT | 54 | 220 |
| 801983 | N/A | N/A | 6293 | 6308 | GATGTCTGGAGGCTCT | 45 | 221 |
| 801989 | N/A | N/A | 6686 | 6701 | AGGCCCACCGCAGCCC | 82 | 222 |
| 801995 | N/A | N/A | 7184 | 7199 | GGGCACTGGAAGCCAA | 64 | 223 |
| 802001 | N/A | N/A | 7671 | 7686 | CCCTTCCTTACGGCCC | 54 | 224 |
| 802007 | N/A | N/A | 8060 | 8075 | CCATCCATCCAAGTCC | 64 | 225 |
| 802013 | N/A | N/A | 8392 | 8407 | AGGACCCAGGTCGCTG | 62 | 226 |
| 802018 | N/A | N/A | 8950 | 8965 | GC CATGTCCAGGGTCC | 58 | 227 |
| 802024 | N/A | N/A | 9368 | 9383 | CAGCAGGGTCCGGACC | 96 | 228 |
| 802030 | N/A | N/A | 9974 | 9989 | GGTGTGCCCAACCTGC | 45 | 229 |
| 802036 | N/A | N/A | 10580 | 10595 | CGCTCTGTCGAGTGCA | 65 | 230 |
| 802041 | N/A | N/A | 10994 | 11009 | ACCCCCCCCCGCAGCC | 79 | 231 |
| 802047 | N/A | N/A | 11564 | 11579 | GACCCGCGCAGCCTCC | 59 | 232 |
| 802053 | N/A | N/A | 12363 | 12378 | AGACAGGCTCAGTGCA | 45 | 233 |
| 802059 | N/A | N/A | 12957 | 12972 | CCCTCCCACACGCCGG | 71 | 234 |
| 802065 | N/A | N/A | 14038 | 14053 | CCGACCCACCCCAGCG | 59 | 235 |
| 802071 | N/A | N/A | 14618 | 14633 | GTGGAGGACACAGAGA | 70 | 236 |
| 802077 | N/A | N/A | 15878 | 15893 | GTCGGCCTGGCATGGG | 78 | 237 |
| 802083 | N/A | N/A | 16311 | 16326 | GGGCACTCCATCCCCT | 92 | 238 |
| 802089 | N/A | N/A | 16795 | 16810 | AGGCATCCCCTCAGCT | 65 | 239 |
| 802095 | N/A | N/A | 17525 | 17540 | TTCATAGACTTTCCCT | 38 | 240 |
| 802101 | N/A | N/A | 18385 | 18400 | GCCCCGAGGGTGGAGG | 90 | 241 |
| 802107* | N/A | N/A | 18818 | 18833 | CCCCCATGCACCGTGC | 83 | 242 |
| 802113* | N/A | N/A | 19156 | 19171 | CAGCAGTGCCCACGGC | 66 | 243 |

TABLE 4
Reduction of SPDEF RNA by 4 μM 3-10-3 cEt gapmers
with a phosphorothioate backbone

| | SEQ | SEQ | SEQ | SEQ | | | |
|---|---|---|---|---|---|---|---|
| | ID | ID | ID | ID | | | |
| | NO: 1 | NO: 1 | NO: 2 | NO: 2 | | | |
| Compound | Start | Stop | Start | Stop | | SPDEF (% | SEQ |
| Number | Site | Site | Site | Site | Sequence (5′ to 3′) | UTC) | ID NO |
| 652522 | 1362 | 1377 | 19647 | 19662 | TCTTGTAATACTGGCG | 62 | 16 |
| 791876* | 1223 | 1238 | 18711 | 18726 | GAAGCGGCCATAGCTG | 79 | 244 |
| 791902 | 1360 | 1375 | 19645 | 19660 | TTGTAATACTGGCGGA | 59 | 245 |
| 801674 | 5 | 20 | 1672 | 1687 | CAGCCGCGAGATGAAG | 80 | 246 |
| 801679 | 39 | 54 | 1706 | 1721 | GCAGAGTGCAGGAATG | 85 | 247 |
| 801685 | 54 | 69 | 1721 | 1736 | GGCAGTGTGGACACGG | 68 | 248 |
| 801691 | 115 | 130 | 1782 | 1797 | AAGGCCCAACCTGAGG | 87 | 249 |
| 801697 | 132 | 147 | 1799 | 1814 | CCTGCTGGCACCGTGG | 83 | 250 |
| 801703 | 235 | 250 | 1902 | 1917 | ACTCAGGTTGGCCACT | 65 | 251 |
| 801709 | 357 | 372 | 2024 | 2039 | CTGCAGTGCCAACTTC | 59 | 252 |
| 801715 | 406 | 421 | 13517 | 13532 | GGGCTGGCGGCTGTGT | 91 | 253 |
| 801721 | 425 | 440 | 13536 | 13551 | GCCCATGCCGCTGCTG | 60 | 254 |
| 801727 | 492 | 507 | 13603 | 13618 | GCGACACCGTGTCGGG | 48 | 255 |
| 801733 | 515 | 530 | 13626 | 13641 | TGCCGCCTTCTCCAAG | 70 | 256 |
| 801739 | 552 | 567 | 13663 | 13678 | GACTCCAGTCCCGTCT | 113 | 257 |
| 801745 | 590 | 605 | 13701 | 13716 | GGCGGACAGGCCCTGC | 104 | 258 |
| 801751 | 611 | 626 | 13722 | 13737 | GTCAAAGTAGGAGAGG | 57 | 259 |
| 801757 | 669 | 684 | 13780 | 13795 | CCCGACTGCTGGCCCC | 47 | 260 |
| 801763 | 702 | 717 | 13813 | 13828 | CCGGGCACTGCTCAGG | 68 | 261 |
| 801769 | 737 | 752 | 13848 | 13863 | GTCCAGGCTGCCCGCT | 94 | 262 |
| 801775 | 763 | 778 | 13874 | 13889 | TCCAAGGTCAGCCCGC | 57 | 263 |
| 801781 | 792 | 807 | 13903 | 13918 | TGGACTGCACCTGCTC | 50 | 264 |
| 801787 | 817 | 832 | 13928 | 13943 | TCCTTGAGCACTTCGC | 61 | 265 |
| 801793 | 836 | 851 | 13947 | 13962 | CTTGCAGGCCGTCTCG | 73 | 266 |
| 801798 | 863 | 878 | N/A | N/A | CATGGGATCTGCGGTG | 72 | 267 |
| 801804 | 918 | 933 | 16867 | 16882 | GGTATTGGTGCTCTGT | 49 | 268 |
| 801810 | 973 | 988 | 16922 | 16937 | GCGCACAGCTCCTTGC | 89 | 269 |
| 801816 | 999 | 1014 | 16948 | 16963 | GCTGGCGGAACTGCTC | 80 | 270 |
| 801820 | 1065 | 1080 | N/A | N/A | TCATCCAGGCCGCTGA | 54 | 271 |
| 801826 | 1092 | 1107 | 18462 | 18477 | GAATCGCCCCAGGTGA | 74 | 272 |
| 801832 | 1109 | 1124 | N/A | N/A | GGTCGAGGCACAGTAG | 95 | 273 |
| 801838 | 1142 | 1157 | 18630 | 18645 | GTCCACCTCGCTGTCG | 64 | 274 |
| 801844 | 1158 | 1173 | 18646 | 18661 | GCCCGGAGCATGATGA | 113 | 275 |
| 801850 | 1181 | 1196 | 18669 | 18684 | GAACTGCCACAGGTGG | 49 | 276 |
| 801859* | 1242 | 1257 | 18730 | 18745 | CCTTGTTGAGCCACCT | 22 | 277 |
| 801865* | 1301 | 1316 | 19586 | 19601 | GCGGATGCCCCACAGC | 23 | 278 |
| 801871* | 1319 | 1334 | 19604 | 19619 | CATGGCGGGACGGTTC | 17 | 279 |
| 801877* | 1346 | 1361 | 19631 | 19646 | GATGGAGCGGCTCAGC | 61 | 280 |
| 801882 | 1381 | 1396 | 19666 | 19681 | GGCTTCCGGATGATGC | 74 | 281 |
| 801888 | 1411 | 1426 | 19696 | 19711 | AACTGGTAGACGAGGC | 57 | 282 |
| 801894 | 1520 | 1535 | 19805 | 19820 | ACTGCCCGTTTTCCCC | 42 | 283 |
| 801900 | 1601 | 1616 | 19886 | 19901 | CCCAGAGGACCCATAT | 75 | 284 |
| 801906 | 1684 | 1699 | 19969 | 19984 | GGGAGCAGCCCTGTCT | 69 | 285 |
| 801912 | 1758 | 1773 | 20043 | 20058 | CCTGTGGCCTTTGTCG | 63 | 286 |
| 801918 | 1881 | 1896 | 20166 | 20181 | TATTATCCATTCCCGG | 55 | 287 |
| 801924 | N/A | N/A | 18603 | 18618 | CTCACTGGTCGAGGCT | 98 | 288 |
| 801930 | N/A | N/A | 2124 | 2139 | TCGCCCACCCCCCAGC | 38 | 289 |
| 801936 | N/A | N/A | 2550 | 2565 | GTCCCGAACTGGACCC | 72 | 290 |
| 801942 | N/A | N/A | 3082 | 3097 | CCAGCCCTGGCCGAGG | 61 | 291 |
| 801948 | N/A | N/A | 3502 | 3517 | TGGATACCCCCACGGG | 51 | 292 |
| 801954 | N/A | N/A | 4074 | 4089 | CCGGCCCCCGCACCCG | 67 | 293 |
| 801960 | N/A | N/A | 4665 | 4680 | GCCCAGGGCAACTCGG | 78 | 294 |
| 801966 | N/A | N/A | 5156 | 5171 | CCGGTTCCCACCTGTG | 56 | 295 |
| 801972 | N/A | N/A | 5662 | 5677 | ACACGGATGTCACCGG | 49 | 296 |
| 801978 | N/A | N/A | 6010 | 6025 | GCCCTGGTTGAGCCCA | 52 | 297 |
| 801984 | N/A | N/A | 6338 | 6353 | GCTGACACTTTTGGCA | 74 | 298 |
| 801990 | N/A | N/A | 6834 | 6849 | GCTGGCAGACCCGGCA | 87 | 299 |
| 801996 | N/A | N/A | 7249 | 7264 | GGGTGAGGCTTTGTGG | 77 | 300 |
| 802002 | N/A | N/A | 7743 | 7758 | GGGAGGACCTTGCTGC | 68 | 301 |
| 802008 | N/A | N/A | 8098 | 8113 | CCCATGTGGCCTACTG | 58 | 302 |
| 802014 | N/A | N/A | 8485 | 8500 | CCACACCCCAACTGGC | 75 | 303 |
| 802019 | N/A | N/A | 8996 | 9011 | GGCCACTGCTCCGTAG | 74 | 304 |
| 802025 | N/A | N/A | 9512 | 9527 | TCCCAGTGGCTGGTGC | 76 | 305 |
| 802031 | N/A | N/A | 10041 | 10056 | CTCCGTCCCCAAGGCA | 50 | 306 |
| 802037 | N/A | N/A | 10621 | 10636 | GCAGCACAGGCCTTAC | 56 | 307 |
| 802042 | N/A | N/A | 11105 | 11120 | TGCTGTGGGCCCACAT | 86 | 308 |
| 802048 | N/A | N/A | 11628 | 11643 | CCCTACTGGGACAGCA | 48 | 309 |
| 802054 | N/A | N/A | 12447 | 12462 | GACTGGAGAGGTGCGC | 73 | 310 |
| 802060 | N/A | N/A | 13158 | 13173 | TGCGCCATTTGGCGGA | 84 | 311 |
| 802066 | N/A | N/A | 14186 | 14201 | GGGACCCTAGGCTGGC | 68 | 312 |
| 802072 | N/A | N/A | 15430 | 15445 | CTCAATTCCCCCGTCC | 51 | 313 |
| 802078 | N/A | N/A | 16014 | 16029 | GGCTGCCCCCACTTAA | 71 | 314 |
| 802084 | N/A | N/A | 16412 | 16427 | ACCCCTCAAGGAACCA | 54 | 315 |
| 802090 | N/A | N/A | 17021 | 17036 | CAGCCATGCCACATCC | 106 | 316 |
| 802096 | N/A | N/A | 17659 | 17674 | GGCCACTGTGGACACG | 81 | 317 |
| 802102 | N/A | N/A | 18428 | 18443 | GGCCGCTGCAGGGCAA | 67 | 318 |
| 802108* | N/A | N/A | 18897 | 18912 | CAGGGCTGTCCCATGA | 96 | 319 |
| 802114* | N/A | N/A | 19282 | 19297 | AACCTCCCGGTACAGG | 70 | 320 |

TABLE 5
Reduction of SPDEF RNA by 4 μM 3-10-3 cEt gapmers
with a phosphorothioate backbone

| | SEQ | SEQ | SEQ | SEQ | | | |
|---|---|---|---|---|---|---|---|
| | ID | ID | ID | ID | | | |
| | NO: 1 | NO: 1 | NO: 2 | NO: 2 | | | |
| Compound | Start | Stop | Start | Stop | | SPDEF (% | SEQ |
| Number | Site | Site | Site | Site | Sequence (5′ to 3′) | UTC) | ID NO |
| 652504* | 1225 | 1240 | 18713 | 18728 | ATGAAGCGGCCATAGC | 113 | 321 |
| 652522 | 1362 | 1377 | 19647 | 19662 | TCTTGTAATACTGGCG | 60 | 16 |
| 791799 | 838 | 853 | 13949 | 13964 | AGCTTGCAGGCCGTCT | 82 | 322 |
| 791904 | 1363 | 1378 | 19648 | 19663 | TTCTTGTAATACTGGC | 53 | 323 |
| 801675 | 7 | 22 | 1674 | 1689 | GACAGCCGCGAGATGA | 74 | 324 |
| 801680 | 41 | 56 | 1708 | 1723 | CGGCAGAGTGCAGGAA | 63 | 325 |
| 801686 | 70 | 85 | 1737 | 1752 | AGGACTGGGTCTGTGG | 65 | 326 |
| 801692 | 118 | 133 | 1785 | 1800 | GGCAAGGCCCAACCTG | 85 | 327 |
| 801698 | 134 | 149 | 1801 | 1816 | TGCCTGCTGGCACCGT | 70 | 328 |
| 801704 | 237 | 252 | 1904 | 1919 | GCACTCAGGTTGGCCA | 89 | 329 |
| 801710 | 359 | 374 | 2026 | 2041 | TGCTGCAGTGCCAACT | 87 | 330 |
| 801716 | 410 | 425 | 13521 | 13536 | GTTTGGGCTGGCGGCT | 89 | 331 |
| 801722 | 443 | 458 | 13554 | 13569 | CAGACCCGGGCTGGCG | 63 | 332 |
| 801728 | 494 | 509 | 13605 | 13620 | CCGCGACACCGTGTCG | 58 | 333 |
| 801734 | 521 | 536 | 13632 | 13647 | CCCCGCTGCCGCCTTC | 61 | 334 |
| 801740 | 555 | 570 | 13666 | 13681 | TGGGACTCCAGTCCCG | 86 | 335 |
| 801746 | 593 | 608 | 13704 | 13719 | GAAGGCGGACAGGCCC | 96 | 336 |
| 801752 | 616 | 631 | 13727 | 13742 | AGCATGTCAAAGTAGG | 51 | 337 |
| 801758 | 671 | 686 | 13782 | 13797 | CTCCCGACTGCTGGCC | 78 | 338 |
| 801764 | 706 | 721 | 13817 | 13832 | ATGACCGGGCACTGCT | 61 | 339 |
| 801770 | 748 | 763 | 13859 | 13874 | CCGGGCACCAAGTCCA | 94 | 340 |
| 801776 | 776 | 791 | 13887 | 13902 | CAGCGAGTGCTCCTCC | 64 | 341 |
| 801782 | 797 | 812 | 13908 | 13923 | CACCATGGACTGCACC | 60 | 342 |
| 801788 | 819 | 834 | 13930 | 13945 | TGTCCTTGAGCACTTC | 58 | 343 |
| 801799 | 865 | 880 | N/A | N/A | TCCATGGGATCTGCGG | 80 | 344 |
| 801805 | 921 | 936 | 16870 | 16885 | GCCGGTATTGGTGCTC | 103 | 345 |
| 801811 | 983 | 998 | 16932 | 16947 | CTCCGACATGGCGCAC | 72 | 346 |
| 801817 | 1001 | 1016 | 16950 | 16965 | GCGCTGGCGGAACTGC | 82 | 347 |
| 801821 | 1075 | 1090 | 18445 | 18460 | GTCCGCTCTTTCATCC | 66 | 348 |
| 801827 | 1094 | 1109 | 18464 | 18479 | GTGAATCGCCCCAGGT | 66 | 349 |
| 801833 | 1113 | 1128 | N/A | N/A | CACTGGTCGAGGCACA | 102 | 350 |
| 801839 | 1145 | 1160 | 18633 | 18648 | TGAGTCCACCTCGCTG | 77 | 351 |
| 801845 | 1163 | 1178 | 18651 | 18666 | GGGCTGCCCGGAGCAT | 83 | 352 |
| 801851 | 1195 | 1210 | 18683 | 18698 | AGCAACTCCTTGAGGA | 58 | 353 |
| 801860* | 1257 | 1272 | N/A | N/A | TGAAGATGCCCTTCTC | 45 | 354 |
| 801866* | 1304 | 1319 | 19589 | 19604 | CTTGCGGATGCCCCAC | 42 | 355 |
| 801872* | 1322 | 1337 | 19607 | 19622 | GTTCATGGCGGGACGG | 16 | 356 |
| 801878* | 1348 | 1363 | 19633 | 19648 | CGGATGGAGCGGCTCA | 107 | 357 |
| 801883 | 1383 | 1398 | 19668 | 19683 | CTGGCTTCCGGATGAT | 81 | 358 |
| 801889 | 1416 | 1431 | 19701 | 19716 | GCACGAACTGGTAGAC | 55 | 359 |
| 801895 | 1524 | 1539 | 19809 | 19824 | GCAGACTGCCCGTTTT | 49 | 360 |
| 801901 | 1610 | 1625 | 19895 | 19910 | CCCGAAGGCCCCAGAG | 68 | 361 |
| 801907 | 1732 | 1747 | 20017 | 20032 | CTTCTGTAGGCTCTGC | 36 | 362 |
| 801913 | 1760 | 1775 | 20045 | 20060 | TGCCTGTGGCCTTTGT | 68 | 363 |
| 801919 | 1894 | 1909 | 20179 | 20194 | TCTCTAGTATCTTTAT | 51 | 364 |
| 801925 | N/A | N/A | 5755 | 5770 | CTCCCAGCTTGCCACA | 70 | 365 |
| 801931 | N/A | N/A | 2207 | 2222 | GGGATCCAGGTCACAG | 59 | 366 |
| 801937 | N/A | N/A | 2627 | 2642 | AGCGGTGACCCCAGCC | 57 | 367 |
| 801943 | N/A | N/A | 3146 | 3161 | GAGCAGCTGGTGATGG | 86 | 368 |
| 801949 | N/A | N/A | 3627 | 3642 | GTGCAGCCCTATTCCC | 106 | 369 |
| 801955 | N/A | N/A | 4125 | 4140 | TGCCCTCTAGGAGGAA | 78 | 370 |
| 801961 | N/A | N/A | 4778 | 4793 | CCCAACCCCGGCTGCT | 66 | 371 |
| 801967 | N/A | N/A | 5341 | 5356 | GCGCCCTGATCCTCAG | 83 | 372 |
| 801973 | N/A | N/A | 5729 | 5744 | CGTGAGGTTTCCTGGG | 51 | 373 |
| 801979 | N/A | N/A | 6060 | 6075 | CCGCTCAACCTTCAGG | 75 | 374 |
| 801985 | N/A | N/A | 6374 | 6389 | GGGCTCCCTTGTAAGC | 62 | 375 |
| 801991 | N/A | N/A | 6904 | 6919 | GGCACCTGTCCATGCG | 64 | 376 |
| 801997 | N/A | N/A | 7297 | 7312 | GCTAGTGGGCCCAGGA | 71 | 377 |
| 802003 | N/A | N/A | 7795 | 7810 | TCTTGCCCTGCTGTTC | 68 | 378 |
| 802009 | N/A | N/A | 8160 | 8175 | CCCCCAGCCGGCCTCA | 56 | 379 |
| 802015 | N/A | N/A | 8563 | 8578 | TGCCACTACCCTGCCT | 80 | 380 |
| 802020 | N/A | N/A | 9054 | 9069 | GAGGTGCCCACAGTCA | 61 | 381 |
| 802026 | N/A | N/A | 9650 | 9665 | CTGACTGGGCTCCTTG | 61 | 382 |
| 802032 | N/A | N/A | 10157 | 10172 | CCCCACCAAGCCTCGG | 35 | 383 |
| 802038 | N/A | N/A | 10724 | 10739 | GGCAGGTGGCAGCTTT | 53 | 384 |
| 802043 | N/A | N/A | 11249 | 11264 | CCCATTCAAGGGCTCC | 38 | 385 |
| 802049 | N/A | N/A | 11777 | 11792 | GGAGACTCCGCAGTCT | 66 | 386 |
| 802055 | N/A | N/A | 12531 | 12546 | CCCCACGGGCCGCCCC | 49 | 387 |
| 802061 | N/A | N/A | 13352 | 13367 | GGTTGGGCAGACAGGC | 51 | 388 |
| 802067 | N/A | N/A | 14279 | 14294 | GTGGCGGGAGCAGAGT | 77 | 389 |
| 802073 | N/A | N/A | 15564 | 15579 | GCCCTAGGAGGTCCCC | 81 | 390 |
| 802079 | N/A | N/A | 16052 | 16067 | GGTCCAGCCAGTGTCC | 75 | 391 |
| 802085 | N/A | N/A | 16489 | 16504 | CCTCAGCCCTAGAGGG | 98 | 392 |
| 802091 | N/A | N/A | 17089 | 17104 | GCCCTAGCAGAGGGCA | 99 | 393 |
| 802097 | N/A | N/A | 17999 | 18014 | GGCTGACACGCAGCCA | 92 | 394 |
| 802103 | N/A | N/A | 18514 | 18529 | CCCACCCGAGCCCCCG | 48 | 395 |
| 802109* | N/A | N/A | 18973 | 18988 | CTGTGCAGTACTAAAA | 61 | 396 |
| 802115* | N/A | N/A | 19319 | 19334 | GGCCCCAGTGAATGGC | 70 | 397 |

TABLE 3
Reduction of SPDEF RNA by 4 μM 3-10-3 cEt
gapmers with a phosphorothioate backbone

| | SEQ ID | SEQ ID | SEQ ID | SEQ ID | | | |
|---|---|---|---|---|---|---|---|
| | NO: 1 | NO: 1 | NO: 2 | NO: 2 | | | |
| Compound | Start | Stop | Start | Stop | | SPDEF | SEQ |
| Number | Site | Site | Site | Site | Sequence (5′ to 3′) | (% UTC) | ID NO |
| 652518* | 1350 | 1365 | 19635 | 19650 | GGCGGATGGAGCGGCT | 121 | 398 |
| 652522 | 1362 | 1377 | 19647 | 19662 | TCTTGTAATACTGGCG | 54 | 16 |
| 791907 | 1367 | 1382 | 19652 | 19667 | GCcCTTCTTGTAATAC | 93 | 399 |
| 801676 | 10 | 25 | 1677 | 1692 | TCAGACAGCCGCGAGA | 73 | 400 |
| 801681 | 43 | 58 | 1710 | 1725 | CACGGCAGAGTGCAGG | 81 | 401 |
| 801687 | 72 | 87 | 1739 | 1754 | GGAGGACTGGGTCTGT | 85 | 402 |
| 801693 | 120 | 135 | 1787 | 1802 | GTGGCAAGGCCCAACC | 82 | 403 |
| 801699 | 164 | 179 | 1831 | 1846 | GTAGGGAGTCCCCTAC | 76 | 404 |
| 801705 | 241 | 256 | 1908 | 1923 | GGCAGCACTCAGGTTG | 83 | 405 |
| 801711 | 388 | 403 | 2055 | 2070 | GTTAGCTGCCTGGTGC | 78 | 406 |
| 801717 | 413 | 428 | 13524 | 13539 | GCTGTTTGGGCTGGCG | 86 | 407 |
| 801723 | 448 | 463 | 13559 | 13574 | CTGCTCAGACCCGGGC | 60 | 408 |
| 801729 | 496 | 511 | 13607 | 13622 | GTCCGCGACACCGTGT | 69 | 409 |
| 801735 | 536 | 551 | 13647 | 13662 | CTCGAGACCCACTGCC | 71 | 410 |
| 801741 | 557 | 572 | 13668 | 13683 | ACTGGGACTCCAGTCC | 56 | 411 |
| 801747 | 595 | 610 | 13706 | 13721 | TAGAAGGCGGACAGGC | 79 | 412 |
| 801753 | 622 | 637 | 13733 | 13748 | GGGTACAGCATGTCAA | 92 | 413 |
| 801759 | 676 | 691 | 13787 | 13802 | GGCTCCTCCCGACTGC | 59 | 414 |
| 801765 | 710 | 725 | 13821 | 13836 | GTCAATGACCGGGCAC | 62 | 415 |
| 801771 | 751 | 766 | 13862 | 13877 | CCGCCGGGCACCAAGT | 90 | 416 |
| 801777 | 777 | 792 | 13888 | 13903 | CCAGCGAGTGCTCCTC | 53 | 417 |
| 801783 | 799 | 814 | 13910 | 13925 | ACCACCATGGACTGCA | 65 | 418 |
| 801789 | 824 | 839 | 13935 | 13950 | CTCGATGTCCTTGAGC | 84 | 419 |
| 801794 | 846 | 861 | 13957 | 13972 | TGTTGAGCAGCTTGCA | 87 | 420 |
| 801800 | 871 | 886 | 16820 | 16835 | CTCCAGTCCATGGGAT | 92 | 421 |
| 801806 | 923 | 938 | 16872 | 16887 | CAGCCGGTATTGGTGC | 83 | 422 |
| 801812 | 985 | 1000 | 16934 | 16949 | TCCTCCGACATGGCGC | 75 | 423 |
| 801818 | 1028 | 1043 | 16977 | 16992 | GTGCAGCACATCCCCA | 85 | 424 |
| 801822 | 1078 | 1093 | 18448 | 18463 | GAAGTCCGCTCTTTCA | 80 | 425 |
| 801828 | 1098 | 1113 | 18468 | 18483 | AGTAGTGAATCGCCCC | 51 | 426 |
| 801834 | 1116 | 1131 | 18604 | 18619 | CCTCACTGGTCGAGGC | 113 | 427 |
| 801840 | 1147 | 1162 | 18635 | 18650 | GATGAGTCCACCTCGC | 64 | 428 |
| 801846 | 1167 | 1182 | 18655 | 18670 | GGATGGGCTGCCCGGA | 64 | 429 |
| 801852 | 1199 | 1214 | 18687 | 18702 | GAGTAGCAACTCCTTG | 61 | 430 |
| 801855* | 1229 | 1244 | 18717 | 18732 | CCTAATGAAGCGGCCA | 91 | 431 |
| 801861* | 1261 | 1276 | N/A | N/A | ATTTTGAAGATGCCCT | 66 | 432 |
| 801867* | 1306 | 1321 | 19591 | 19606 | TTCTTGCGGATGCCCC | 27 | 433 |
| 801873* | 1326 | 1341 | 19611 | 19626 | CGTAGTTCATGGCGGG | 25 | 434 |
| 801884 | 1386 | 1401 | 19671 | 19686 | TGTCTGGCTTCCGGAT | 80 | 435 |
| 801890 | 1419 | 1434 | 19704 | 19719 | GGTGCACGAACTGGTA | 87 | 436 |
| 801896 | 1526 | 1541 | 19811 | 19826 | GAGCAGACTGCCCGTT | 58 | 437 |
| 801902 | 1614 | 1629 | 19899 | 19914 | GGGTCCCGAAGGCCCC | 72 | 438 |
| 801908 | 1735 | 1750 | 20020 | 20035 | GCCCTTCTGTAGGCTC | 76 | 439 |
| 801914 | 1766 | 1781 | 20051 | 20066 | CTGGACTGCCTGTGGC | 47 | 440 |
| 801920 | 1896 | 1911 | 20181 | 20196 | GTTCTCTAGTATCTTT | 50 | 441 |
| 801926 | N/A | N/A | 5128 | 5143 | AGACACATCCCCCTTT | 92 | 442 |
| 801932 | N/A | N/A | 2307 | 2322 | CCAGGCCTTGCCGGGC | 80 | 443 |
| 801938 | N/A | N/A | 2686 | 2701 | AGACCAGGACCCAAGG | 55 | 444 |
| 801944 | N/A | N/A | 3240 | 3255 | GGCCTGCCCGTCTGGT | 95 | 445 |
| 801950 | N/A | N/A | 3697 | 3712 | GTGGGTTCTCCCGGTT | 26 | 446 |
| 801956 | N/A | N/A | 4167 | 4182 | TCTAGCCCAGTCCAGG | 74 | 447 |
| 801962 | N/A | N/A | 4877 | 4892 | GTCCCATCCGACCCCC | 57 | 448 |
| 801968 | N/A | N/A | 5400 | 5415 | CCACACACCTGGTTGT | 87 | 449 |
| 801974 | N/A | N/A | 5773 | 5788 | GCCCCGCATACGCCGT | 42 | 450 |
| 801980 | N/A | N/A | 6143 | 6158 | GCCCAGACAAACCTGG | 113 | 451 |
| 801986 | N/A | N/A | 6483 | 6498 | TGTTAGCCCTGGCACT | 72 | 452 |
| 801992 | N/A | N/A | 6977 | 6992 | TGCCGGGCCCTCCCAG | 62 | 453 |
| 801998 | N/A | N/A | 7364 | 7379 | GGCCAACTGTCCCCCT | 66 | 454 |
| 802004 | N/A | N/A | 7871 | 7886 | GCCGCAGTAGCATGTC | 73 | 455 |
| 802010 | N/A | N/A | 8252 | 8267 | GCCCGCCCAGAGCCCA | 57 | 456 |
| 802016 | N/A | N/A | 8665 | 8680 | CACCTTGGGCCCCTTC | 89 | 457 |
| 802021 | N/A | N/A | 9118 | 9133 | CAGTGATGGTCCACCC | 44 | 458 |
| 802027 | N/A | N/A | 9698 | 9713 | GGTGCATGCTCTGGCC | 58 | 459 |
| 802033 | N/A | N/A | 10233 | 10248 | GCTCGGGCTCCTTCAC | 69 | 460 |
| 802039 | N/A | N/A | 10792 | 10807 | GGTAGGACAGGAGGCA | 69 | 461 |
| 802044 | N/A | N/A | 11346 | 11361 | TGCCCAACCTTCCCAG | 69 | 462 |
| 802050 | N/A | N/A | 11870 | 11885 | GCCGTCTGGGCCAGCA | 70 | 463 |
| 802056 | N/A | N/A | 12715 | 12730 | CGGCCACCCGGAGGCA | 90 | 464 |
| 802062 | N/A | N/A | 13451 | 13466 | GGGCCGCTAAGCTGGT | 65 | 465 |
| 802068 | N/A | N/A | 14411 | 14426 | GGCCTCATGCGGATGG | 76 | 466 |
| 802074 | N/A | N/A | 15643 | 15658 | ACTCAGCAGCCCCGCC | 64 | 467 |
| 802080 | N/A | N/A | 16100 | 16115 | GATAGGCTGGTGGGCA | 82 | 468 |
| 802086 | N/A | N/A | 16557 | 16572 | GCCCGCCTCACCCAGG | 62 | 469 |
| 802092 | N/A | N/A | 17167 | 17182 | GTGCACCAGGATCCAG | 71 | 470 |
| 802098 | N/A | N/A | 18131 | 18146 | GTCTCTGACAGGGTCC | 26 | 471 |
| 802104 | N/A | N/A | 18556 | 18571 | ATGGGAGGCCAGTCCC | 88 | 472 |
| 802110* | N/A | N/A | 19066 | 19081 | TTCTCAGGCAGTTCGG | 47 | 473 |
| 802116* | N/A | N/A | 19418 | 19433 | CCCCCTGCTCGGGTGG | 87 | 474 |

TABLE 7
Reduction of SPDEF RNA by 4 μM 3-10-3 cEt
gapmers with a phosphorothioate backbone

| | SEQ ID | SEQ ID | SEQ ID | SEQ ID | | | |
|---|---|---|---|---|---|---|---|
| | NO: 1 | NO: 1 | NO: 2 | NO: 2 | | | |
| Compound | Start | Stop | Start | Stop | | SPDEF | SEQ |
| Number | Site | Site | Site | Site | Sequence (5′ to 3′) | (% UTC) | ID NO |
| 791817 | 977 | 992 | 16926 | 16941 | CATGGCGCACAGCTCC | 77 | 475 |
| 801766 | 712 | 727 | 13823 | 13838 | CTGTCAATGACCGGGC | 49 | 33 |
| 802094 | N/A | N/A | 17292 | 17307 | CACGGTTGTCCCCAGC | 33 | 163 |
| 832823 | 1 | 16 | 1668 | 1683 | CGCGAGATGAAGAGTT | 86 | 476 |
| 832839 | 74 | 89 | 1741 | 1756 | TTGGAGGACTGGGTCT | 126 | 477 |
| 832855 | 163 | 178 | 1830 | 1845 | TAGGGAGTCCCCTACC | 91 | 478 |
| 832871 | 355 | 370 | 2022 | 2037 | GCAGTGCCAACTTCAG | 54 | 479 |
| 832887 | 409 | 424 | 13520 | 13535 | TTTGGGCTGGCGGCTG | 104 | 480 |
| 832903 | 449 | 464 | 13560 | 13575 | GCTGCTCAGACCCGGG | 79 | 481 |
| 832919 | 537 | 552 | 13648 | 13663 | TCTCGAGACCCACTGC | 90 | 482 |
| 832935 | 560 | 575 | 13671 | 13686 | TGGACTGGGACTCCAG | 96 | 483 |
| 832951 | 613 | 628 | 13724 | 13739 | ATGTCAAAGTAGGAGA | 82 | 484 |
| 832967 | 679 | 694 | 13790 | 13805 | GGTGGCTCCTCCCGAC | 54 | 485 |
| 832983 | 757 | 772 | 13868 | 13883 | GTCAGCCCGCCGGGCA | 91 | 486 |
| 832999 | 809 | 824 | 13920 | 13935 | CACTTCGCCCACCACC | 62 | 487 |
| 833015 | 832 | 847 | 13943 | 13958 | CAGGCCGTCTCGATGT | 66 | 488 |
| 833030 | 868 | 883 | 16817 | 16832 | CAGTCCATGGGATCTG | 87 | 489 |
| 833060 | 1029 | 1044 | 16978 | 16993 | CGTGCAGCACATCCCC | 60 | 490 |
| 833072 | 1079 | 1094 | 18449 | 18464 | TGAAGTCCGCTCTTTC | 81 | 491 |
| 833088 | 1104 | 1119 | N/A | N/A | AGGCACAGTAGTGAAT | 111 | 492 |
| 833104 | 1135 | 1150 | 18623 | 18638 | TCGCTGTCGGTCCAGC | 111 | 493 |
| 833120 | 1161 | 1176 | 18649 | 18664 | GCTGCCCGGAGCATGA | 72 | 494 |
| 833132* | 1224 | 1239 | 18712 | 18727 | TGAAGCGGCCATAGCT | 105 | 495 |
| 833146* | 1273 | 1288 | 19558 | 19573 | GCTGAGTCCTCAATTT | 73 | 496 |
| 833153 | 1389 | 1404 | 19674 | 19689 | AGATGTCTGGCTTCCG | 60 | 497 |
| 833169 | 1570 | 1585 | 19855 | 19870 | GGTTGCCCCTCCCTGA | 63 | 498 |
| 833185 | 1736 | 1751 | 20021 | 20036 | TGCCCTTCTGTAGGCT | 91 | 499 |
| 833201 | 1882 | 1897 | 20167 | 20182 | TTATTATCCATTCCCG | 40 | 500 |
| 833217 | N/A | N/A | 4997 | 5012 | GCGCTGGAGCCCCGGG | 101 | 501 |
| 833233 | N/A | N/A | 5024 | 5039 | TGGGCCTTGCCCCGCA | 92 | 502 |
| 833249 | N/A | N/A | 5081 | 5096 | AGGAGCTAGGTCCCAG | 67 | 503 |
| 833265 | N/A | N/A | 5162 | 5177 | TCAGGACCGGTTCCCA | 54 | 504 |
| 833281 | N/A | N/A | 5232 | 5247 | CTGACAGGCTAAGAAC | 87 | 505 |
| 833297 | N/A | N/A | 5292 | 5307 | TGTTAGGACAAAGTGA | 90 | 506 |
| 833313 | N/A | N/A | 5351 | 5366 | AAACATTCCTGCGCCC | 93 | 507 |
| 833329 | N/A | N/A | 5394 | 5409 | ACCTGGTTGTTGGTCT | 91 | 508 |
| 833345 | N/A | N/A | 5460 | 5475 | ATCTGCCGTGTTTCTG | 80 | 509 |
| 833361 | N/A | N/A | 5502 | 5517 | TCTCCCGAGAGGTGTG | 102 | 510 |
| 833377 | N/A | N/A | 5529 | 5544 | GGACAGCGATGTGAGA | 93 | 511 |
| 833393 | N/A | N/A | 5614 | 5629 | CGATCCTCTTGGCCTC | 89 | 512 |
| 833409 | N/A | N/A | 5631 | 5646 | GCCTGAGGCCCCTTGG | 97 | 513 |
| 833425 | N/A | N/A | 5666 | 5681 | GAACACACGGATGTCA | 101 | 514 |
| 833457 | N/A | N/A | 4854 | 4869 | TCACACTAAGGTCCCT | 72 | 515 |
| 833473 | N/A | N/A | 4879 | 4894 | CGGTCCCATCCGACCC | 95 | 516 |
| 833489 | N/A | N/A | 4909 | 4924 | TGGTGCGCCGTCATAA | 50 | 517 |
| 833505 | N/A | N/A | 18272 | 18287 | AAGCTGGTTACAAGAA | 94 | 518 |
| 833521 | N/A | N/A | 2230 | 2245 | GGGCAAGGAATTCTGA | 102 | 519 |
| 833537 | N/A | N/A | 2871 | 2886 | TACTTCCGCGCACACA | 111 | 520 |
| 833553 | N/A | N/A | 3376 | 3391 | CTCCGAGAATGCCCCC | 56 | 521 |
| 833569 | N/A | N/A | 3705 | 3720 | GGTAAAAAGTGGGTTC | 62 | 522 |
| 833585 | N/A | N/A | 3902 | 3917 | AGGAAAAGTGACCCGC | 113 | 523 |
| 833601 | N/A | N/A | 4435 | 4450 | GTCAAGAGTATGTCTT | 51 | 524 |
| 833617 | N/A | N/A | 5830 | 5845 | CGGTACACTCCTTGAA | 90 | 525 |
| 833633 | N/A | N/A | 6279 | 6294 | CTGAAAGACTCAGCCC | 75 | 526 |
| 833649 | N/A | N/A | 6705 | 6720 | CCGCAGCCTGGAGGTA | 86 | 527 |
| 833665 | N/A | N/A | 6985 | 7000 | AACTGCTTTGCCGGGC | 58 | 528 |
| 833681 | N/A | N/A | 7624 | 7639 | CGCTGGACAGACCCACC | 99 | 529 |
| 833697 | N/A | N/A | 8263 | 8278 | ACCCAATGCCAGCCCG | 66 | 530 |
| 833713 | N/A | N/A | 8589 | 8604 | AAGGAGAGATTTAGTG | 94 | 531 |
| 833729 | N/A | N/A | 9170 | 9185 | TCCTAGGCTCGCCTCA | 74 | 532 |
| 833745 | N/A | N/A | 9593 | 9608 | CCCCACTGTTCATATC | 108 | 533 |
| 833761 | N/A | N/A | 10154 | 10169 | CACCAAGCCTCGGTCC | 82 | 534 |
| 833776 | N/A | N/A | 11026 | 11041 | GCCCTACCCGCTAGGT | 75 | 535 |
| 833792 | N/A | N/A | 11478 | 11493 | CGGTAGGGCCACAGCT | 78 | 536 |
| 833808 | N/A | N/A | 11919 | 11934 | TCCTTTCTCGAGGGTT | 71 | 537 |
| 833824 | N/A | N/A | 12481 | 12496 | CAATAGCAGAGTGCAC | 70 | 538 |
| 833840 | N/A | N/A | 12888 | 12903 | CTCAACACTCTCAAGG | 87 | 539 |
| 833856 | N/A | N/A | 13154 | 13169 | CCATTTGGCGGATGAG | 86 | 540 |
| 833872 | N/A | N/A | 13447 | 13462 | CGCTAAGCTGGTTATG | 104 | 541 |
| 833888 | N/A | N/A | 14251 | 14266 | AGCGAAGTCCAAGAGG | 98 | 542 |
| 833904 | N/A | N/A | 14672 | 14687 | GGATTGATGAGCAAAA | 47 | 543 |
| 833920 | N/A | N/A | 15669 | 15684 | GGCGACAGCAGGACAG | 106 | 544 |
| 833936 | N/A | N/A | 16159 | 16174 | TCCTAGATGTCCCCCT | 63 | 545 |
| 833952 | N/A | N/A | 16629 | 16644 | GGCGAGAGGAAGGAAC | 91 | 546 |
| 833968 | N/A | N/A | 17599 | 17614 | TAATACTCTGCTACTA | 96 | 547 |
| 833984 | N/A | N/A | 18212 | 18227 | CCGTAAAGGGCTTGCA | 77 | 548 |
| 834000* | N/A | N/A | 19020 | 19035 | AATATGAGATGGTGGA | 84 | 549 |
| 834016* | N/A | N/A | 19358 | 19373 | CGGTGAGGTTAAAGAG | 100 | 550 |

TABLE 8
Reduction of SPDEF RNA by 4 μM 3-10-3 cEt
gapmers with a phosphorothioate backbone

| | SEQ ID | SEQ ID | SEQ ID | SEQ ID | | | |
|---|---|---|---|---|---|---|---|
| | NO: 1 | NO: 1 | NO: 2 | NO: 2 | | | |
| Compound | Start | Stop | Start | Stop | | SPDEF | SEQ |
| Number | Site | Site | Site | Site | Sequence (5′ to 3′) | (% UTC) | ID NO |
| 652464 | 978 | 993 | 16927 | 16942 | ACATGGCGCACAGCTC | 70 | 551 |
| 801766 | 712 | 727 | 13823 | 13838 | CTGTCAATGACCGGGC | 64 | 33 |
| 802094 | N/A | N/A | 17292 | 17307 | CACGGTTGTCCCCAGC | 34 | 163 |
| 832824 | 6 | 21 | 1673 | 1688 | ACAGCCGCGAGATGAA | 71 | 552 |
| 832840 | 114 | 129 | 1781 | 1796 | AGGCCCAACCTGAGGG | 108 | 553 |
| 832856 | 165 | 180 | 1832 | 1847 | TGTAGGGAGTCCCCTA | 77 | 554 |
| 832872 | 356 | 371 | 2023 | 2038 | TGCAGTGCCAACTTCA | 67 | 555 |
| 832888 | 411 | 426 | 13522 | 13537 | TGTTTGGGCTGGCGGC | 110 | 556 |
| 832904 | 451 | 466 | 13562 | 13577 | ACGCTGCTCAGACCCG | 46 | 557 |
| 832920 | 538 | 553 | 13649 | 13664 | CTCTCGAGACCCACTG | 75 | 558 |
| 832936 | 561 | 576 | 13672 | 13687 | GTGGACTGGGACTCCA | 95 | 559 |
| 832952 | 614 | 629 | 13725 | 13740 | CATGTCAAAGTAGGAG | 93 | 560 |
| 832968 | 704 | 719 | 13815 | 13830 | GACCGGGCACTGCTCA | 65 | 561 |
| 832984 | 759 | 774 | 13870 | 13885 | AGGTCAGCCCGCCGGG | 79 | 562 |
| 833000 | 810 | 825 | 13921 | 13936 | GCACTTCGCCCACCAC | 54 | 563 |
| 833016 | 834 | 849 | 13945 | 13960 | TGCAGGCCGTCTCGAT | 87 | 564 |
| 833031 | 869 | 884 | 16818 | 16833 | CCAGTCCATGGGATCT | 67 | 565 |
| 833061 | 1051 | 1066 | 17000 | 17015 | GACTTCCAGATGTCCA | 106 | 566 |
| 833073 | 1080 | 1095 | 18450 | 18465 | GTGAAGTCCGCTCTTT | 103 | 567 |
| 833089 | 1105 | 1120 | N/A | N/A | GAGGCACAGTAGTGAA | 116 | 568 |
| 833105 | 1136 | 1151 | 18624 | 18639 | CTCGCTGTCGGTCCAG | 81 | 569 |
| 833121 | 1162 | 1177 | 18650 | 18665 | GGCTGCCCGGAGCATG | 88 | 570 |
| 833133* | 1227 | 1242 | 18715 | 18730 | TAATGAAGCGGCCATA | 101 | 571 |
| 833147* | 1314 | 1329 | 19599 | 19614 | CGGGACGGTTCTTGCG | 4 | 572 |
| 833154 | 1391 | 1406 | 19676 | 19691 | GGAGATGTCTGGCTTC | 86 | 573 |
| 833170 | 1574 | 1589 | 19859 | 19874 | AGTTGGTTGCCCCTCC | 63 | 574 |
| 833186 | 1737 | 1752 | 20022 | 20037 | CTGCCCTTCTGTAGGC | 93 | 575 |
| 833202 | 1897 | 1912 | 20182 | 20197 | AGTTCTCTAGTATCTT | 40 | 576 |
| 833218 | N/A | N/A | 5009 | 5024 | AACCAGTCTCAGGCGC | 74 | 577 |
| 833234 | N/A | N/A | 5025 | 5040 | CTGGGCCTTGCCCCGC | 117 | 578 |
| 833250 | N/A | N/A | 5082 | 5097 | GAGGAGCTAGGTCCCA | 90 | 579 |
| 833266 | N/A | N/A | 5163 | 5178 | ATCAGGACCGGTTCCC | 51 | 580 |
| 833282 | N/A | N/A | 5235 | 5250 | GTCCTGACAGGCTAAG | 83 | 581 |
| 833298 | N/A | N/A | 5293 | 5308 | GTGTTAGGACAAAGTG | 78 | 582 |
| 833314 | N/A | N/A | 5352 | 5367 | AAAACATTCCTGCGCC | 82 | 583 |
| 833330 | N/A | N/A | 5395 | 5410 | CACCTGGTTGTTGGTC | 103 | 584 |
| 833346 | N/A | N/A | 5461 | 5476 | CATCTGCCGTGTTTCT | 66 | 585 |
| 833362 | N/A | N/A | 5504 | 5519 | TGTCTCCCGAGAGGTG | 88 | 586 |
| 833378 | N/A | N/A | 5530 | 5545 | AGGACAGCGATGTGAG | 87 | 587 |
| 833394 | N/A | N/A | 5615 | 5630 | CCGATCCTCTTGGCCT | 121 | 588 |
| 833410 | N/A | N/A | 5650 | 5665 | CCGGAGCTCTGCTGCT | 107 | 589 |
| 833426 | N/A | N/A | 5667 | 5682 | AGAACACACGGATGTC | 85 | 590 |
| 833458 | N/A | N/A | 4855 | 4870 | GTCACACTAAGGTCCC | 81 | 591 |
| 833474 | N/A | N/A | 4880 | 4895 | CCGGTCCCATCCGACC | 93 | 592 |
| 833490 | N/A | N/A | 4910 | 4925 | GTGGTGCGCCGTCATA | 49 | 593 |
| 833506 | N/A | N/A | 18273 | 18288 | CAAGCTGGTTACAAGA | 73 | 594 |
| 833522 | N/A | N/A | 2274 | 2289 | ATGTAGAGTTGGCCCA | 69 | 595 |
| 833538 | N/A | N/A | 2873 | 2888 | CATACTTCCGCGCACA | 115 | 596 |
| 833554 | N/A | N/A | 3421 | 3436 | ACGCAGTGAGACCACC | 65 | 597 |
| 833570 | N/A | N/A | 3709 | 3724 | CAGTGGTAAAAAGTGG | 76 | 598 |
| 833586 | N/A | N/A | 3916 | 3931 | TTGCAAGTACAGTGAG | 62 | 599 |
| 833602 | N/A | N/A | 4447 | 4462 | GTTAAATGGGCTGTCA | 87 | 600 |
| 833618 | N/A | N/A | 5833 | 5848 | CGCCGGTACACTCCTT | 61 | 601 |
| 833634 | N/A | N/A | 6355 | 6370 | GGCATACTCCATTTAC | 66 | 602 |
| 833650 | N/A | N/A | 6715 | 6730 | GCACAGGTGCCCGCAG | 90 | 603 |
| 833666 | N/A | N/A | 7034 | 7049 | GGCACTACTTCCAGCG | 87 | 604 |
| 833682 | N/A | N/A | 7627 | 7642 | GCCCGCTGGACAGACC | 87 | 605 |
| 833698 | N/A | N/A | 8275 | 8290 | CTCAATCCTGAGACCC | 61 | 606 |
| 833714 | N/A | N/A | 8673 | 8688 | GGATTAGCCACCTTGG | 64 | 607 |
| 833730 | N/A | N/A | 9193 | 9208 | CCTAATAGCTCCCTCC | 86 | 608 |
| 833746 | N/A | N/A | 9630 | 9645 | TCTAAAGTCTGTCCCC | 105 | 609 |
| 833762 | N/A | N/A | 10172 | 10187 | GCCAAGGAATCTACTC | 51 | 610 |
| 833777 | N/A | N/A | 11084 | 11099 | ACTCAGGCAGTGCCAA | 65 | 611 |
| 833793 | N/A | N/A | 11486 | 11501 | CTCCACTTCGGTAGGG | 85 | 612 |
| 833809 | N/A | N/A | 11942 | 11957 | TGTTAAGGGCAAGTTA | 82 | 613 |
| 833825 | N/A | N/A | 12483 | 12498 | ACCAATAGCAGAGTGC | 54 | 614 |
| 833841 | N/A | N/A | 12940 | 12955 | GAGTAGGCCAGCCCTT | 73 | 615 |
| 833857 | N/A | N/A | 13156 | 13171 | CGCCATTTGGCGGATG | 83 | 616 |
| 833873 | N/A | N/A | 13450 | 13465 | GGCCGCTAAGCTGGTT | 102 | 617 |
| 833889 | N/A | N/A | 14256 | 14271 | GGGAGAGCGAAGTCCA | 91 | 618 |
| 833905 | N/A | N/A | 14674 | 14689 | ATGGATTGATGAGCAA | 62 | 619 |
| 833921 | N/A | N/A | 15674 | 15689 | GGAGAGGCGACAGCAG | 89 | 620 |
| 833937 | N/A | N/A | 16166 | 16181 | GGCTAGGTCCTAGATG | 94 | 621 |
| 833953 | N/A | N/A | 16696 | 16711 | GGGATAGGTCAGCCCC | 73 | 622 |
| 833969 | N/A | N/A | 17602 | 17617 | GTGTAATACTCTGCTA | 79 | 623 |
| 833985 | N/A | N/A | 18214 | 18229 | GGCCGTAAAGGGCTTG | 87 | 624 |
| 834001* | N/A | N/A | 19061 | 19076 | AGGCAGTTCGGCCTGT | 109 | 625 |
| 834017* | N/A | N/A | 19373 | 19388 | CCCAAGGTGTAGTTGC | 89 | 626 |

TABLE 9
Reduction of SPDEF RNA by 4 μM 3-10-3 cEt
gapmers with a phosphorothioate backbone

| | SEQ ID | SEQ ID | SEQ ID | SEQ ID | | | |
|---|---|---|---|---|---|---|---|
| | NO: 1 | NO: 1 | NO: 2 | NO: 2 | | | |
| Compound | Start | Stop | Start | Stop | | SPDEF | SEQ |
| Number | Site | Site | Site | Site | Sequence (5′ to 3′) | (% UTC) | ID NO |
| 652495 | 1164 | 1179 | 18652 | 18667 | TGGGCTGCCCGGAGCA | 71 | 627 |
| 791897* | 1352 | 1367 | 19637 | 19652 | CTGGCGGATGGAGCGG | 84 | 628 |
| 801766 | 712 | 727 | 13823 | 13838 | CTGTCAATGACCGGGC | 52 | 33 |
| 802094 | N/A | N/A | 17292 | 17307 | CACGGTTGTCCCCAGC | 28 | 163 |
| 832825 | 8 | 23 | 1675 | 1690 | AGACAGCCGCGAGATG | 100 | 629 |
| 832841 | 116 | 131 | 1783 | 1798 | CAAGGCCCAACCTGAG | 82 | 630 |
| 832857 | 166 | 181 | 1833 | 1848 | CTGTAGGGAGTCCCCT | 86 | 631 |
| 832873 | 358 | 373 | 2025 | 2040 | GCTGCAGTGCCAACTT | 64 | 632 |
| 832889 | 412 | 427 | 13523 | 13538 | CTGTTTGGGCTGGCGG | 102 | 633 |
| 832905 | 452 | 467 | 13563 | 13578 | TACGCTGCTCAGACCC | 40 | 634 |
| 832921 | 539 | 554 | 13650 | 13665 | TCTCTCGAGACCCACT | 57 | 635 |
| 832937 | 568 | 583 | 13679 | 13694 | GTGGCGGGTGGACTGG | 75 | 636 |
| 832953 | 615 | 630 | 13726 | 13741 | GCATGTCAAAGTAGGA | 67 | 637 |
| 832969 | 705 | 720 | 13816 | 13831 | TGACCGGGCACTGCTC | 68 | 638 |
| 832985 | 760 | 775 | 13871 | 13886 | AAGGTCAGCCCGCCGG | 118 | 639 |
| 833001 | 811 | 826 | 13922 | 13937 | AGCACTTCGCCCACCA | 57 | 640 |
| 833017 | 835 | 850 | 13946 | 13961 | TTGCAGGCCGTCTCGA | 99 | 641 |
| 833032 | 870 | 885 | 16819 | 16834 | TCCAGTCCATGGGATC | 101 | 642 |
| 833046 | 979 | 994 | 16928 | 16943 | GACATGGCGCACAGCT | 76 | 643 |
| 833062 | 1053 | 1068 | 17002 | 17017 | CTGACTTCCAGATGTC | 125 | 644 |
| 833074 | 1082 | 1097 | 18452 | 18467 | AGGTGAAGTCCGCTCT | 95 | 645 |
| 833090 | 1107 | 1122 | N/A | N/A | TCGAGGCACAGTAGTG | 102 | 646 |
| 833106 | 1137 | 1152 | 18625 | 18640 | CCTCGCTGTCGGTCCA | 122 | 647 |
| 833134* | 1228 | 1243 | 18716 | 18731 | CTAATGAAGCGGCCAT | 77 | 648 |
| 833155 | 1392 | 1407 | 19677 | 19692 | GGGAGATGTCTGGCTT | 109 | 649 |
| 833171 | 1578 | 1593 | 19863 | 19878 | GGGCAGTTGGTTGCCC | 73 | 650 |
| 833187 | 1738 | 1753 | 20023 | 20038 | ACTGCCCTTCTGTAGG | 80 | 651 |
| 833203 | 1898 | 1913 | 20183 | 20198 | CAGTTCTCTAGTATCT | 64 | 652 |
| 833219 | N/A | N/A | 5010 | 5025 | CAACCAGTCTCAGGCG | 97 | 653 |
| 833235 | N/A | N/A | 5047 | 5062 | GCTACCCCAGGAGCAG | 82 | 654 |
| 833251 | N/A | N/A | 5083 | 5098 | GGAGGAGCTAGGTCCC | 120 | 655 |
| 833267 | N/A | N/A | 5164 | 5179 | TATCAGGACCGGTTCC | 83 | 656 |
| 833283 | N/A | N/A | 5236 | 5251 | TGTCCTGACAGGCTAA | 75 | 657 |
| 833299 | N/A | N/A | 5294 | 5309 | GGTGTTAGGACAAAGT | 80 | 658 |
| 833315 | N/A | N/A | 5353 | 5368 | AAAAACATTCCTGCGC | 89 | 659 |
| 833331 | N/A | N/A | 5396 | 5411 | ACACCTGGTTGTTGGT | 120 | 660 |
| 833347 | N/A | N/A | 5462 | 5477 | CCATCTGCCGTGTTTC | 86 | 661 |
| 833363 | N/A | N/A | 5505 | 5520 | CTGTCTCCCGAGAGGT | 81 | 662 |
| 833379 | N/A | N/A | 5531 | 5546 | CAGGACAGCGATGTGA | 83 | 663 |
| 833395 | N/A | N/A | 5616 | 5631 | GCCGATCCTCTTGGCC | 101 | 664 |
| 833411 | N/A | N/A | 5651 | 5666 | ACCGGAGCTCTGCTGC | 90 | 665 |
| 833427 | N/A | N/A | 5668 | 5683 | GAGAACACACGGATGT | 74 | 666 |
| 833459 | N/A | N/A | 4856 | 4871 | AGTCACACTAAGGTCC | 77 | 667 |
| 833475 | N/A | N/A | 4881 | 4896 | CCCGGTCCCATCCGAC | 89 | 668 |
| 833491 | N/A | N/A | 4911 | 4926 | GGTGGTGCGCCGTCAT | 49 | 669 |
| 833507 | N/A | N/A | 18274 | 18289 | GCAAGCTGGTTACAAG | 92 | 670 |
| 833523 | N/A | N/A | 2278 | 2293 | CAGCATGTAGAGTTGG | 95 | 671 |
| 833539 | N/A | N/A | 2876 | 2891 | ACACATACTTCCGCGC | 130 | 672 |
| 833555 | N/A | N/A | 3425 | 3440 | CCCTACGCAGTGAGAC | 71 | 673 |
| 833571 | N/A | N/A | 3718 | 3733 | CAACGACCTCAGTGGT | 63 | 674 |
| 833587 | N/A | N/A | 3925 | 3940 | GACAAGGTGTTGCAAG | 85 | 675 |
| 833603 | N/A | N/A | 4449 | 4464 | CTGTTAAATGGGCTGT | 60 | 676 |
| 833619 | N/A | N/A | 5836 | 5851 | AATCGCCGGTACACTC | 94 | 677 |
| 833635 | N/A | N/A | 6361 | 6376 | AGCAAAGGCATACTCC | 34 | 678 |
| 833651 | N/A | N/A | 6716 | 6731 | AGCACAGGTGCCCGCA | 74 | 679 |
| 833667 | N/A | N/A | 7041 | 7056 | CCTCACTGGCACTACT | 98 | 680 |
| 833683 | N/A | N/A | 7640 | 7655 | GAGCACCACTTCTGCC | 47 | 681 |
| 833699 | N/A | N/A | 8298 | 8313 | GGTAAATGTATCCTCA | 53 | 682 |
| 833715 | N/A | N/A | 8810 | 8825 | GGATTAAGGCTCAGCG | 48 | 683 |
| 833731 | N/A | N/A | 9276 | 9291 | GGGCACAACATGGCTA | 88 | 684 |
| 833747 | N/A | N/A | 9796 | 9811 | ATAGATGCGGACAGTG | 83 | 685 |
| 833763 | N/A | N/A | 10184 | 10199 | CTATACCTAAATGCCA | 106 | 686 |
| 833778 | N/A | N/A | 11087 | 11102 | TTTACTCAGGCAGTGC | 60 | 687 |
| 833794 | N/A | N/A | 11552 | 11567 | CTCCGTATGCAGCTGG | 76 | 688 |
| 833810 | N/A | N/A | 11949 | 11964 | ATAAACCTGTTAAGGG | 110 | 689 |
| 833826 | N/A | N/A | 12524 | 12539 | GGCCGCCCCGGCTTGG | 103 | 690 |
| 833842 | N/A | N/A | 12966 | 12981 | GGGTAGAAACCCTCCC | 113 | 691 |
| 833858 | N/A | N/A | 13227 | 13242 | ATGTACTGTGCTTAAA | 90 | 692 |
| 833874 | N/A | N/A | 13504 | 13519 | TGTCTACGGAAATGAA | 98 | 693 |
| 833890 | N/A | N/A | 14313 | 14328 | TAGCAAATGTTGTGGG | 95 | 694 |
| 833906 | N/A | N/A | 14694 | 14709 | TGCTATCCTAGCATCT | 93 | 695 |
| 833922 | N/A | N/A | 15678 | 15693 | AGCTGGAGAGGCGACA | 84 | 696 |
| 833938 | N/A | N/A | 16277 | 16292 | GGGCTAGACGCACAGG | 71 | 697 |
| 833954 | N/A | N/A | 16740 | 16755 | ACCCATGGGAACCTGT | 88 | 698 |
| 833970 | N/A | N/A | 17607 | 17622 | GAGCAGTGTAATACTC | 97 | 699 |
| 833986 | N/A | N/A | 18384 | 18399 | CCCCGAGGGTGGAGGA | 110 | 700 |
| 834002* | N/A | N/A | 19065 | 19080 | TCTCAGGCAGTTCGGC | 84 | 701 |
| 834018* | N/A | N/A | 19439 | 19454 | AGGGACCCCGTGCAGA | 125 | 702 |

TABLE 10
Reduction of SPDEF RNA by 4 μM 3-10-3 cEt
gapmers with a phosphorothioate backbone

| | SEQ ID | SEQ ID | SEQ ID | SEQ ID | | | |
|---|---|---|---|---|---|---|---|
| | NO: 1 | NO: 1 | NO: 2 | NO: 2 | | | |
| Compound | Start | Stop | Start | Stop | | SPDEF | SEQ |
| Number | Site | Site | Site | Site | Sequence (5′ to 3′) | (% UTC) | ID NO |
| 652444 | 837 | 852 | 13948 | 13963 | GCTTGCAGGCCGTCTC | 122 | 703 |
| 652478 | 1054 | 1069 | N/A | N/A | GCTGACTTCCAGATGT | 98 | 704 |
| 791870 | 1165 | 1180 | 18653 | 18668 | ATGGGCTGCCCGGAGC | 65 | 705 |
| 791898 | 1354 | 1369 | 19639 | 19654 | TACTGGCGGATGGAGC | 127 | 706 |
| 801766 | 712 | 727 | 13823 | 13838 | CTGTCAATGACCGGGC | 46 | 33 |
| 802094 | N/A | N/A | 17292 | 17307 | CACGGTTGTCCCCAGC | 29 | 163 |
| 832826 | 9 | 24 | 1676 | 1691 | CAGACAGCCGCGAGAT | 84 | 707 |
| 832842 | 117 | 132 | 1784 | 1799 | GCAAGGCCCAACCTGA | 91 | 708 |
| 832858 | 168 | 183 | 1835 | 1850 | GCCTGTAGGGAGTCCC | 110 | 709 |
| 832874 | 389 | 404 | 2056 | 2071 | TGTTAGCTGCCTGGTG | 113 | 710 |
| 832890 | 414 | 429 | 13525 | 13540 | TGCTGTTTGGGCTGGC | 86 | 711 |
| 832906 | 454 | 469 | 13565 | 13580 | GATACGCTGCTCAGAC | 61 | 712 |
| 832922 | 540 | 555 | 13651 | 13666 | GTCTCTCGAGACCCAC | 65 | 713 |
| 832938 | 583 | 598 | 13694 | 13709 | AGGCCCTGCTCGGGCG | 92 | 714 |
| 832954 | 620 | 635 | 13731 | 13746 | GTACAGCATGTCAAAG | 96 | 715 |
| 832970 | 707 | 722 | 13818 | 13833 | AATGACCGGGCACTGC | 67 | 716 |
| 832986 | 762 | 777 | 13873 | 13888 | CCAAGGTCAGCCCGCC | 72 | 717 |
| 833002 | 813 | 828 | 13924 | 13939 | TGAGCACTTCGCCCAC | 93 | 718 |
| 833033 | 888 | 903 | 16837 | 16852 | TCTGCACATTGCTGGG | 59 | 719 |
| 833047 | 980 | 995 | 16929 | 16944 | CGACATGGCGCACAGC | 66 | 720 |
| 833075 | 1084 | 1099 | 18454 | 18469 | CCAGGTGAAGTCCGCT | 83 | 721 |
| 833091 | 1108 | 1123 | N/A | N/A | GTCGAGGCACAGTAGT | 89 | 722 |
| 833107 | 1139 | 1154 | 18627 | 18642 | CACCTCGCTGTCGGTC | 89 | 723 |
| 833135* | 1230 | 1245 | 18718 | 18733 | ACCTAATGAAGCGGCC | 90 | 724 |
| 833156 | 1410 | 1425 | 19695 | 19710 | ACTGGTAGACGAGGCG | 66 | 725 |
| 833172 | 1594 | 1609 | 19879 | 19894 | GACCCATATCCCCCTG | 86 | 726 |
| 833188 | 1747 | 1762 | 20032 | 20047 | TGTCGAGTCACTGCCC | 48 | 727 |
| 833204 | 1899 | 1914 | 20184 | 20199 | TCAGTTCTCTAGTATC | 50 | 728 |
| 833220 | N/A | N/A | 5011 | 5026 | GCAACCAGTCTCAGGC | 66 | 729 |
| 833236 | N/A | N/A | 5048 | 5063 | TGCTACCCCAGGAGCA | 105 | 730 |
| 833252 | N/A | N/A | 5084 | 5099 | AGGAGGAGCTAGGTCC | 93 | 731 |
| 833268 | N/A | N/A | 5165 | 5180 | TTATCAGGACCGGTTC | 89 | 732 |
| 833284 | N/A | N/A | 5237 | 5252 | CTGTCCTGACAGGCTA | 70 | 733 |
| 833300 | N/A | N/A | 5295 | 5310 | GGGTGTTAGGACAAAG | 94 | 734 |
| 833316 | N/A | N/A | 5371 | 5386 | CTTGATGGGCTGAAGG | 117 | 735 |
| 833332 | N/A | N/A | 5397 | 5412 | CACACCTGGTTGTTGG | 113 | 736 |
| 833348 | N/A | N/A | 5463 | 5478 | TCCATCTGCCGTGTTT | 76 | 737 |
| 833364 | N/A | N/A | 5506 | 5521 | ACTGTCTCCCGAGAGG | 97 | 738 |
| 833380 | N/A | N/A | 5532 | 5547 | GCAGGACAGCGATGTG | 78 | 739 |
| 833396 | N/A | N/A | 5617 | 5632 | GGCCGATCCTCTTGGC | 101 | 740 |
| 833412 | N/A | N/A | 5652 | 5667 | CACCGGAGCTCTGCTG | 75 | 741 |
| 833428 | N/A | N/A | 5674 | 5689 | GGGCATGAGAACACAC | 89 | 742 |
| 833460 | N/A | N/A | 4857 | 4872 | GAGTCACACTAAGGTC | 73 | 743 |
| 833476 | N/A | N/A | 4882 | 4897 | TCCCGGTCCCATCCGA | 112 | 744 |
| 833492 | N/A | N/A | 4912 | 4927 | TGGTGGTGCGCCGTCA | 59 | 745 |
| 833508 | N/A | N/A | 18275 | 18290 | GGCAAGCTGGTTACAA | 104 | 746 |
| 833524 | N/A | N/A | 2359 | 2374 | TGCACGGCGGCCTCCC | 63 | 747 |
| 833540 | N/A | N/A | 2910 | 2925 | GCAACACGCACGCGCA | 120 | 748 |
| 833556 | N/A | N/A | 3445 | 3460 | CTCAAAGGCGAGGGTG | 98 | 749 |
| 833572 | N/A | N/A | 3722 | 3737 | CTCACAACGACCTCAG | 38 | 750 |
| 833588 | N/A | N/A | 3941 | 3956 | CTAACCTTGTTTCACA | 94 | 751 |
| 833604 | N/A | N/A | 4535 | 4550 | GAAAAGGTTTGATCCC | 94 | 752 |
| 833620 | N/A | N/A | 5839 | 5854 | CCAAATCGCCGGTACA | 89 | 753 |
| 833636 | N/A | N/A | 6393 | 6408 | ACGCAGAGGTGGACAC | 118 | 754 |
| 833652 | N/A | N/A | 6748 | 6763 | CCCCACAGCAGTTGCC | 101 | 755 |
| 833668 | N/A | N/A | 7072 | 7087 | CTGCATGGGCAGCCTG | 119 | 756 |
| 833684 | N/A | N/A | 7668 | 7683 | TTCCTTACGGCCCTCC | 85 | 757 |
| 833700 | N/A | N/A | 8356 | 8371 | CCATATCCTGCTTGGT | 76 | 758 |
| 833716 | N/A | N/A | 8812 | 8827 | CTGGATTAAGGCTCAG | 80 | 759 |
| 833732 | N/A | N/A | 9299 | 9314 | TTTCATACCTGCCCCT | 77 | 760 |
| 833748 | N/A | N/A | 9801 | 9816 | GCTTTATAGATGCGGA | 46 | 761 |
| 833764 | N/A | N/A | 10186 | 10201 | CCCTATACCTAAATGC | 94 | 762 |
| 833779 | N/A | N/A | 11089 | 11104 | TATTTACTCAGGCAGT | 90 | 763 |
| 833795 | N/A | N/A | 11560 | 11575 | CGCGCAGCCTCCGTAT | 89 | 764 |
| 833811 | N/A | N/A | 11951 | 11966 | AGATAAACCTGTTAAG | 111 | 765 |
| 833827 | N/A | N/A | 12555 | 12570 | ACACAAGCAGTCAGAG | 104 | 766 |
| 833843 | N/A | N/A | 12983 | 12998 | ACGAGAGGAACAAGGC | 68 | 767 |
| 833859 | N/A | N/A | 13232 | 13247 | CACACATGTACTGTGC | 117 | 768 |
| 833875 | N/A | N/A | 13506 | 13521 | TGTGTCTACGGAAATG | 106 | 769 |
| 833891 | N/A | N/A | 14395 | 14410 | TGCCATCTGAGCCAAG | 85 | 770 |
| 833907 | N/A | N/A | 14707 | 14722 | GTTATATTCAAGGTGC | 43 | 771 |
| 833923 | N/A | N/A | 15701 | 15716 | GGACATGGGTCAGGAC | 50 | 772 |
| 833939 | N/A | N/A | 16280 | 16295 | AAAGGGCTAGACGCAC | 41 | 773 |
| 833955 | N/A | N/A | 16770 | 16785 | CCTGAGAGCACCACCC | 126 | 774 |
| 833971 | N/A | N/A | 17611 | 17626 | TGCAGAGCAGTGTAAT | 104 | 775 |
| 833987 | N/A | N/A | 18472 | 18487 | CCACAGTAGTGAATCG | 91 | 776 |
| 834003* | N/A | N/A | 19118 | 19133 | CCCCATTACAGGTGTC | 68 | 777 |
| 834019* | N/A | N/A | 19442 | 19457 | CCAAGGGACCCCGTGC | 92 | 778 |

TABLE 11
Reduction of SPDEF RNA by 4 μM 3-10-3 cEt
gapmers with a phosphorothioate backbone

| | SEQ ID | SEQ ID | SEQ ID | SEQ ID | | | |
|---|---|---|---|---|---|---|---|
| | NO: 1 | NO: 1 | NO: 2 | NO: 2 | | | |
| Compound | Start | Stop | Start | Stop | | SPDEF | SEQ |
| Number | Site | Site | Site | Site | Sequence (5′ to 3′) | (% UTC) | ID NO |
| 652520 | 1356 | 1371 | 19641 | 19656 | AATACTGGCGGATGGA | 118 | 779 |
| 801766 | 712 | 727 | 13823 | 13838 | CTGTCAATGACCGGGC | 83 | 33 |
| 802094 | N/A | N/A | 17292 | 17307 | CACGGTTGTCCCCAGC | 54 | 163 |
| 832827 | 11 | 26 | 1678 | 1693 | GTCAGACAGCCGCGAG | 95 | 780 |
| 832843 | 119 | 134 | 1786 | 1801 | TGGCAAGGCCCAACCT | 105 | 781 |
| 832859 | 169 | 184 | 1836 | 1851 | TGCCTGTAGGGAGTCC | 133 | 782 |
| 832875 | 391 | 406 | N/A | N/A | TCTGTTAGCTGCCTGG | 101 | 783 |
| 832891 | 418 | 433 | 13529 | 13544 | CCGCTGCTGTTTGGGC | 117 | 784 |
| 832907 | 455 | 470 | 13566 | 13581 | GGATACGCTGCTCAGA | 92 | 785 |
| 832923 | 541 | 556 | 13652 | 13667 | CGTCTCTCGAGACCCA | 86 | 786 |
| 832939 | 585 | 600 | 13696 | 13711 | ACAGGCCCTGCTCGGG | 143 | 787 |
| 832955 | 625 | 640 | 13736 | 13751 | TCAGGGTACAGCATGT | 127 | 788 |
| 832971 | 708 | 723 | 13819 | 13834 | CAATGACCGGGCACTG | 125 | 789 |
| 832987 | 764 | 779 | 13875 | 13890 | CTCCAAGGTCAGCCCG | 99 | 790 |
| 833003 | 814 | 829 | 13925 | 13940 | TTGAGCACTTCGCCCA | 98 | 791 |
| 833018 | 839 | 854 | 13950 | 13965 | CAGCTTGCAGGCCGTC | 96 | 792 |
| 833034 | 915 | 930 | 16864 | 16879 | ATTGGTGCTCTGTCCA | 79 | 793 |
| 833048 | 981 | 996 | 16930 | 16945 | CCGACATGGCGCACAG | 100 | 794 |
| 833063 | 1055 | 1070 | N/A | N/A | CGCTGACTTCCAGATG | 117 | 795 |
| 833076 | 1086 | 1101 | 18456 | 18471 | CCCCAGGTGAAGTCCG | 130 | 796 |
| 833092 | 1110 | 1125 | N/A | N/A | TGGTCGAGGCACAGTA | 128 | 797 |
| 833108 | 1140 | 1155 | 18628 | 18643 | CCACCTCGCTGTCGGT | 95 | 798 |
| 833122 | 1166 | 1181 | 18654 | 18669 | GATGGGCTGCCCGGAG | 109 | 799 |
| 833136* | 1232 | 1247 | 18720 | 18735 | CCACCTAATGAAGCGG | 111 | 800 |
| 833157 | 1412 | 1427 | 19697 | 19712 | GAACTGGTAGACGAGG | 100 | 801 |
| 833173 | 1595 | 1610 | 19880 | 19895 | GGACCCATATCCCCCT | 119 | 802 |
| 833189 | 1748 | 1763 | 20033 | 20048 | TTGTCGAGTCACTGCC | 82 | 803 |
| 833221 | N/A | N/A | 5012 | 5027 | CGCAACCAGTCTCAGG | 83 | 804 |
| 833237 | N/A | N/A | 5049 | 5064 | TTGCTACCCCAGGAGC | 110 | 805 |
| 833253 | N/A | N/A | 5126 | 5141 | ACACATCCCCCTTTTG | 130 | 806 |
| 833269 | N/A | N/A | 5166 | 5181 | GTTATCAGGACCGGTT | 100 | 807 |
| 833285 | N/A | N/A | 5240 | 5255 | GAACTGTCCTGACAGG | 93 | 808 |
| 833301 | N/A | N/A | 5321 | 5336 | TGGAGCACACCTCCAG | 126 | 809 |
| 833317 | N/A | N/A | 5372 | 5387 | TCTTGATGGGCTGAAG | 109 | 810 |
| 833333 | N/A | N/A | 5398 | 5413 | ACACACCTGGTTGTTG | 90 | 811 |
| 833349 | N/A | N/A | 5466 | 5481 | GTCTCCATCTGCCGTG | 127 | 812 |
| 833365 | N/A | N/A | 5507 | 5522 | CACTGTCTCCCGAGAG | 114 | 813 |
| 833381 | N/A | N/A | 5533 | 5548 | GGCAGGACAGCGATGT | 127 | 814 |
| 833397 | N/A | N/A | 5618 | 5633 | TGGCCGATCCTCTTGG | 130 | 815 |
| 833413 | N/A | N/A | 5653 | 5668 | TCACCGGAGCTCTGCT | 77 | 816 |
| 833429 | N/A | N/A | 5675 | 5690 | AGGGCATGAGAACACA | 98 | 817 |
| 833445 | N/A | N/A | 4825 | 4840 | GGATGAGCCTCTCCCT | 123 | 818 |
| 833461 | N/A | N/A | 4858 | 4873 | AGAGTCACACTAAGGT | 102 | 819 |
| 833477 | N/A | N/A | 4883 | 4898 | GTCCCGGTCCCATCCG | 104 | 820 |
| 833493 | N/A | N/A | 4913 | 4928 | CTGGTGGTGCGCCGTC | 83 | 821 |
| 833509 | N/A | N/A | 18276 | 18291 | AGGCAAGCTGGTTACA | 127 | 822 |
| 833525 | N/A | N/A | 2366 | 2381 | CTGCATCTGCACGGCG | 86 | 823 |
| 833541 | N/A | N/A | 2912 | 2927 | ATGCAACACGCACGCG | 107 | 824 |
| 833557 | N/A | N/A | 3464 | 3479 | GTACATGCACTGTCAG | 95 | 825 |
| 833573 | N/A | N/A | 3726 | 3741 | TATACTCACAACGACC | 140 | 826 |
| 833589 | N/A | N/A | 3955 | 3970 | GGCAATAGCCTTGTCT | 92 | 827 |
| 833605 | N/A | N/A | 4612 | 4627 | TGACAGGCCACTCGCT | 93 | 828 |
| 833621 | N/A | N/A | 5841 | 5856 | CCCCAAATCGCCGGTA | 103 | 829 |
| 833637 | N/A | N/A | 6413 | 6428 | CTTAAAGAAGGATGGT | 107 | 830 |
| 833653 | N/A | N/A | 6808 | 6823 | CCTAAGGTTGCCCCTG | 88 | 831 |
| 833669 | N/A | N/A | 7107 | 7122 | ACACATTGCATCAGTG | 99 | 832 |
| 833685 | N/A | N/A | 7686 | 7701 | AGAGAAGTGCCAGACC | 91 | 833 |
| 833701 | N/A | N/A | 8389 | 8404 | ACCCAGGTCGCTGTGC | 118 | 834 |
| 833717 | N/A | N/A | 8828 | 8843 | GGATTAAGCCACATGT | 88 | 835 |
| 833733 | N/A | N/A | 9304 | 9319 | TGGCATTTCATACCTG | 72 | 836 |
| 833749 | N/A | N/A | 9855 | 9870 | CCACATCACCCGCTTT | 90 | 837 |
| 833765 | N/A | N/A | 10214 | 10229 | CTATACCCCACATTCC | 115 | 838 |
| 833780 | N/A | N/A | 11329 | 11344 | GTTACATGGCAGCCCT | 72 | 839 |
| 833796 | N/A | N/A | 11568 | 11583 | GACTGACCCGCGCAGC | 122 | 840 |
| 833812 | N/A | N/A | 11985 | 12000 | ATACAGAGAACCAGTT | 120 | 841 |
| 833828 | N/A | N/A | 12560 | 12575 | CCTCAACACAAGCAGT | 129 | 842 |
| 833844 | N/A | N/A | 12986 | 13001 | AAGACGAGAGGAACAA | 108 | 843 |
| 833860 | N/A | N/A | 13288 | 13303 | ATAGATCGCTCCCTCA | 81 | 844 |
| 833876 | N/A | N/A | 13996 | 14011 | TACGGAAGCAGGCACA | 136 | 845 |
| 833892 | N/A | N/A | 14403 | 14418 | GCGGATGGTGCCATCT | 95 | 846 |
| 833908 | N/A | N/A | 14715 | 14730 | GAGCATCAGTTATATT | 104 | 847 |
| 833924 | N/A | N/A | 15740 | 15755 | ACAGAGTTCAGTGCTG | 128 | 848 |
| 833940 | N/A | N/A | 16288 | 16303 | CACGGAATAAAGGGCT | 104 | 849 |
| 833956 | N/A | N/A | 17078 | 17093 | GGGCAACCTCCTAGCC | 137 | 850 |
| 833972 | N/A | N/A | 17624 | 17639 | ACATACTGTGGTGTGC | 111 | 851 |
| 833988 | N/A | N/A | 18477 | 18492 | GCTCACCACAGTAGTG | 112 | 852 |
| 834004* | N/A | N/A | 19135 | 19150 | TGGCAAGAGCATCCCT | 95 | 853 |
| 834020* | N/A | N/A | 19444 | 19459 | AACCAAGGGACCCCGT | 112 | 854 |

TABLE 12
Reduction of SPDEF RNA by 4 μM 3-10-3 cEt
gapmers with a phosphorothioate backbone

| | SEQ ID | SEQ ID | SEQ ID | SEQ ID | | | |
|---|---|---|---|---|---|---|---|
| | NO: 1 | NO: 1 | NO: 2 | NO: 2 | | | |
| Compound | Start | Stop | Start | Stop | | SPDEF | SEQ |
| Number | Site | Site | Site | Site | Sequence (5′ to 3′) | (% UTC) | ID NO |
| 791901 | 1358 | 1373 | 19643 | 19658 | GTAATACTGGCGGATG | 133 | 855 |
| 801766 | 712 | 727 | 13823 | 13838 | CTGTCAATGACCGGGC | 51 | 33 |
| 802094 | N/A | N/A | 17292 | 17307 | CACGGTTGTCCCCAGC | 32 | 163 |
| 832828 | 13 | 28 | 1680 | 1695 | AAGTCAGACAGCCGCG | 99 | 856 |
| 832844 | 121 | 136 | 1788 | 1803 | CGTGGCAAGGCCCAAC | 95 | 857 |
| 832860 | 170 | 185 | 1837 | 1852 | GTGCCTGTAGGGAGTC | 92 | 858 |
| 832876 | 392 | 407 | N/A | N/A | GTCTGTTAGCTGCCTG | 132 | 859 |
| 832892 | 419 | 434 | 13530 | 13545 | GCCGCTGCTGTTTGGG | 79 | 860 |
| 832908 | 493 | 508 | 13604 | 13619 | CGCGACACCGTGTCGG | 97 | 861 |
| 832924 | 543 | 558 | 13654 | 13669 | CCCGTCTCTCGAGACC | 74 | 862 |
| 832940 | 586 | 601 | 13697 | 13712 | GACAGGCCCTGCTCGG | 123 | 863 |
| 832956 | 626 | 641 | 13737 | 13752 | CTCAGGGTACAGCATG | 104 | 864 |
| 832972 | 709 | 724 | 13820 | 13835 | TCAATGACCGGGCACT | 121 | 865 |
| 832988 | 773 | 788 | 13884 | 13899 | CGAGTGCTCCTCCAAG | 46 | 866 |
| 833004 | 816 | 831 | 13927 | 13942 | CCTTGAGCACTTCGCC | 73 | 867 |
| 833019 | 840 | 855 | 13951 | 13966 | GCAGCTTGCAGGCCGT | 89 | 868 |
| 833035 | 917 | 932 | 16866 | 16881 | GTATTGGTGCTCTGTC | 78 | 869 |
| 833049 | 982 | 997 | 16931 | 16946 | TCCGACATGGCGCACA | 85 | 870 |
| 833064 | 1059 | 1074 | N/A | N/A | AGGCCGCTGACTTCCA | 123 | 871 |
| 833077 | 1088 | 1103 | 18458 | 18473 | CGCCCCAGGTGAAGTC | 117 | 872 |
| 833093 | 1111 | 1126 | N/A | N/A | CTGGTCGAGGCACAGT | 112 | 873 |
| 833109 | 1143 | 1158 | 18631 | 18646 | AGTCCACCTCGCTGTC | 75 | 874 |
| 833123 | 1169 | 1184 | 18657 | 18672 | GTGGATGGGCTGCCCG | 49 | 875 |
| 833137* | 1234 | 1249 | 18722 | 18737 | AGCCACCTAATGAAGC | 101 | 876 |
| 833158 | 1417 | 1432 | 19702 | 19717 | TGCACGAACTGGTAGA | 80 | 877 |
| 833174 | 1596 | 1611 | 19881 | 19896 | AGGACCCATATCCCCC | 75 | 878 |
| 833190 | 1749 | 1764 | 20034 | 20049 | TTTGTCGAGTCACTGC | 62 | 879 |
| 833222 | N/A | N/A | 5013 | 5028 | CCGCAACCAGTCTCAG | 95 | 880 |
| 833238 | N/A | N/A | 5050 | 5065 | CTTGCTACCCCAGGAG | 93 | 881 |
| 833254 | N/A | N/A | 5129 | 5144 | GAGACACATCCCCCTT | 83 | 882 |
| 833270 | N/A | N/A | 5167 | 5182 | GGTTATCAGGACCGGT | 55 | 883 |
| 833286 | N/A | N/A | 5241 | 5256 | AGAACTGTCCTGACAG | 85 | 884 |
| 833302 | N/A | N/A | 5322 | 5337 | CTGGAGCACACCCTCA | 124 | 885 |
| 833318 | N/A | N/A | 5373 | 5388 | ATCTTGATGGGCTGAA | 111 | 886 |
| 833334 | N/A | N/A | 5399 | 5414 | CACACACCTGGTTGTT | 134 | 887 |
| 833350 | N/A | N/A | 5467 | 5482 | TGTCTCCATCTGCCGT | 57 | 888 |
| 833366 | N/A | N/A | 5508 | 5523 | TCACTGTCTCCCGAGA | 57 | 889 |
| 833382 | N/A | N/A | 5534 | 5549 | AGGCAGGACAGCGATG | 103 | 890 |
| 833398 | N/A | N/A | 5619 | 5634 | TTGGCCGATCCTCTTG | 132 | 891 |
| 833414 | N/A | N/A | 5654 | 5669 | GTCACCGGAGCTCTGC | 62 | 892 |
| 833430 | N/A | N/A | 5676 | 5691 | GAGGGCATGAGAACAC | 105 | 893 |
| 833446 | N/A | N/A | 4826 | 4841 | AGGATGAGCCTCTCCC | 124 | 894 |
| 833462 | N/A | N/A | 4859 | 4874 | CAGAGTCACACTAAGG | 75 | 895 |
| 833478 | N/A | N/A | 4884 | 4899 | GGTCCCGGTCCCATCC | 77 | 896 |
| 833494 | N/A | N/A | 4914 | 4929 | CCTGGTGGTGCGCCGT | 82 | 897 |
| 833510 | N/A | N/A | 18277 | 18292 | GAGGCAAGCTGGTTAC | 112 | 898 |
| 833526 | N/A | N/A | 2427 | 2442 | CAGCAAGCCGCTTGTG | 103 | 899 |
| 833542 | N/A | N/A | 2914 | 2929 | ACATGCAACACGCACG | 94 | 900 |
| 833558 | N/A | N/A | 3469 | 3484 | TGCTTGTACATGCACT | 64 | 901 |
| 833574 | N/A | N/A | 3729 | 3744 | CTTTATACTCACAACG | 77 | 902 |
| 833590 | N/A | N/A | 3960 | 3975 | TAACAGGCAATAGCCT | 111 | 903 |
| 833606 | N/A | N/A | 4628 | 4643 | GAAGAGTTGTTCCACC | 74 | 904 |
| 833622 | N/A | N/A | 5892 | 5907 | ATGCAGCCCGGGTCAC | 125 | 905 |
| 833638 | N/A | N/A | 6457 | 6472 | TACGATCCATGACCCT | 69 | 906 |
| 833654 | N/A | N/A | 6831 | 6846 | GGCAGACCCGGCATCT | 99 | 907 |
| 833670 | N/A | N/A | 7109 | 7124 | GAACACATTGCATCAG | 71 | 908 |
| 833686 | N/A | N/A | 7690 | 7705 | CCCAAGAGAAGTGCCA | 94 | 909 |
| 833702 | N/A | N/A | 8396 | 8411 | GCTAAGGACCCAGGTC | 68 | 910 |
| 833718 | N/A | N/A | 8835 | 8850 | CTCTTCTGGATTAAGC | 108 | 911 |
| 833734 | N/A | N/A | 9323 | 9338 | ATCCAAGCTCTAATGA | 113 | 912 |
| 833750 | N/A | N/A | 9881 | 9896 | CACCAGTGCCACGCCC | 79 | 913 |
| 833766 | N/A | N/A | 10234 | 10249 | GGCTCGGGCTCCTTCA | 65 | 914 |
| 833781 | N/A | N/A | 11336 | 11351 | TCCCAGTGTTACATGG | 80 | 915 |
| 833797 | N/A | N/A | 11571 | 11586 | TGAGACTGACCCGCGC | 92 | 916 |
| 833813 | N/A | N/A | 12005 | 12020 | ACAGATATACGCTCCT | 42 | 917 |
| 833829 | N/A | N/A | 12562 | 12577 | GGCCTCAACACAAGCA | 92 | 918 |
| 833845 | N/A | N/A | 13011 | 13026 | GGCTATCATCTTCACC | 66 | 919 |
| 833861 | N/A | N/A | 13291 | 13306 | GAAATAGATCGCTCCC | 73 | 920 |
| 833877 | N/A | N/A | 13999 | 14014 | TCTTACGGAAGCAGGC | 90 | 921 |
| 833893 | N/A | N/A | 14409 | 14424 | CCTCATGCGGATGGTG | 102 | 922 |
| 833909 | N/A | N/A | 15375 | 15390 | CAGAGAGGTAGCTCAT | 75 | 923 |
| 833925 | N/A | N/A | 15775 | 15790 | ATGCATGAAGACCCCT | 103 | 924 |
| 833941 | N/A | N/A | 16291 | 16306 | CTCCACGGAATAAAGG | 125 | 925 |
| 833957 | N/A | N/A | 17083 | 17098 | GCAGAGGGCAACCTCC | 97 | 926 |
| 833973 | N/A | N/A | 17628 | 17643 | CAAGACATACTGTGGT | 49 | 927 |
| 833989 | N/A | N/A | 18497 | 18512 | CTCCACCCTGCCGCTG | 96 | 928 |
| 834005* | N/A | N/A | 19149 | 19164 | GCCCACGGCTCACTTG | 123 | 929 |
| 834021* | N/A | N/A | 19447 | 19462 | GCGAACCAAGGGACCC | 97 | 930 |

TABLE 13
Reduction of SPDEF RNA by 4 μM 3-10-3 cEt
gapmers with a phosphorothioate backbone

| | SEQ ID | SEQ ID | SEQ ID | SEQ ID | | | |
|---|---|---|---|---|---|---|---|
| | NO: 1 | NO: 1 | NO: 2 | NO: 2 | | | |
| Compound | Start | Stop | Start | Stop | | SPDEF | SEQ |
| Number | Site | Site | Site | Site | Sequence (5′ to 3′) | (% UTC) | ID NO |
| 652480 | 1060 | 1075 | N/A | N/A | CAGGCCGCTGACTTCC | 90 | 931 |
| 652521 | 1359 | 1374 | 19644 | 19659 | TGTAATACTGGCGGAT | 72 | 932 |
| 801766 | 712 | 727 | 13823 | 13838 | CTGTCAATGACCGGGC | 61 | 33 |
| 802094 | N/A | N/A | 17292 | 17307 | CACGGTTGTCCCCAGC | 25 | 163 |
| 832829 | 14 | 29 | 1681 | 1696 | GAAGTCAGACAGCCGC | 56 | 933 |
| 832845 | 122 | 137 | 1789 | 1804 | CCGTGGCAAGGCCCAA | 85 | 934 |
| 832861 | 196 | 211 | 1863 | 1878 | GTGGCCCTCTGAGGTC | 86 | 935 |
| 832877 | 394 | 409 | N/A | N/A | GTGTCTGTTAGCTGCC | 97 | 936 |
| 832893 | 421 | 436 | 13532 | 13547 | ATGCCGCTGCTGTTTG | 67 | 937 |
| 832909 | 495 | 510 | 13606 | 13621 | TCCGCGACACCGTGTC | 61 | 938 |
| 832925 | 544 | 559 | 13655 | 13670 | TCCCGTCTCTCGAGAC | 93 | 939 |
| 832941 | 587 | 602 | 13698 | 13713 | GGACAGGCCCTGCTCG | 97 | 940 |
| 832957 | 628 | 643 | 13739 | 13754 | TCCTCAGGGTACAGCA | 87 | 941 |
| 832973 | 711 | 726 | 13822 | 13837 | TGTCAATGACCGGGCA | 58 | 942 |
| 832989 | 774 | 789 | 13885 | 13900 | GCGAGTGCTCCTCCAA | 48 | 943 |
| 833005 | 818 | 833 | 13929 | 13944 | GTCCTTGAGCACTTCG | 82 | 944 |
| 833020 | 841 | 856 | 13952 | 13967 | AGCAGCTTGCAGGCCG | 71 | 945 |
| 833036 | 919 | 934 | 16868 | 16883 | CGGTATTGGTGCTCTG | 88 | 946 |
| 833050 | 984 | 999 | 16933 | 16948 | CCTCCGACATGGCGCA | 113 | 947 |
| 833078 | 1089 | 1104 | 18459 | 18474 | TCGCCCCAGGTGAAGT | 88 | 948 |
| 833094 | 1112 | 1127 | N/A | N/A | ACTGGTCGAGGCACAG | 103 | 949 |
| 833110 | 1146 | 1161 | 18634 | 18649 | ATGAGTCCACCTCGCT | 74 | 950 |
| 833124 | 1196 | 1211 | 18684 | 18699 | TAGCAACTCCTTGAGG | 62 | 951 |
| 833138* | 1235 | 1250 | 18723 | 18738 | GAGCCACCTAATGAAG | 57 | 952 |
| 833159 | 1418 | 1433 | 19703 | 19718 | GTGCACGAACTGGTAG | 104 | 953 |
| 833175 | 1598 | 1613 | 19883 | 19898 | AGAGGACCCATATCCC | 68 | 954 |
| 833191 | 1750 | 1765 | 20035 | 20050 | CTTTGTCGAGTCACTG | 68 | 955 |
| 833223 | N/A | N/A | 5014 | 5029 | CCCGCAACCAGTCTCA | 71 | 956 |
| 833239 | N/A | N/A | 5051 | 5066 | ACTTGCTACCCCAGGA | 55 | 957 |
| 833255 | N/A | N/A | 5130 | 5145 | AGAGACACATCCCCCT | 62 | 958 |
| 833271 | N/A | N/A | 5183 | 5198 | GAGTGGGTTATTAAGG | 83 | 959 |
| 833287 | N/A | N/A | 5275 | 5290 | GGACTCCAACATCACA | 60 | 960 |
| 833303 | N/A | N/A | 5340 | 5355 | CGCCCTGATCCTCAGG | 85 | 961 |
| 833319 | N/A | N/A | 5374 | 5389 | AATCTTGATGGGCTGA | 88 | 962 |
| 833335 | N/A | N/A | 5450 | 5465 | TTTCTGCGGCCCCTCC | 55 | 963 |
| 833351 | N/A | N/A | 5477 | 5492 | TGGTGACTGCTGTCTC | 74 | 964 |
| 833367 | N/A | N/A | 5509 | 5524 | TTCACTGTCTCCCGAG | 85 | 965 |
| 833383 | N/A | N/A | 5535 | 5550 | CAGGCAGGACAGCGAT | 85 | 966 |
| 833399 | N/A | N/A | 5620 | 5635 | CTTGGCCGATCCTCTT | 79 | 967 |
| 833415 | N/A | N/A | 5655 | 5670 | TGTCACCGGAGCTCTG | 78 | 968 |
| 833431 | N/A | N/A | 5677 | 5692 | TGAGGGCATGAGAACA | 100 | 969 |
| 833447 | N/A | N/A | 4827 | 4842 | AAGGATGAGCCTCTCC | 102 | 970 |
| 833463 | N/A | N/A | 4860 | 4875 | GCAGAGTCACACTAAG | 54 | 971 |
| 833479 | N/A | N/A | 4885 | 4900 | AGGTCCCGGTCCCATC | 56 | 972 |
| 833495 | N/A | N/A | 4915 | 4930 | TCCTGGTGGTGCGCCG | 81 | 973 |
| 833511 | N/A | N/A | 18278 | 18293 | GGAGGCAAGCTGGTTA | 85 | 974 |
| 833527 | N/A | N/A | 2512 | 2527 | CATCAAGCTCCAGCAA | 80 | 975 |
| 833543 | N/A | N/A | 2916 | 2931 | GCACATGCAACACGCA | 108 | 976 |
| 833559 | N/A | N/A | 3503 | 3518 | CTGGATACCCCCACGG | 74 | 977 |
| 833575 | N/A | N/A | 3746 | 3761 | GGTTTCAGGGCTATTC | 34 | 978 |
| 833591 | N/A | N/A | 3962 | 3977 | TTTAACAGGCAATAGC | 92 | 979 |
| 833607 | N/A | N/A | 4646 | 4661 | GTGCAAAGTTTGCTTT | 55 | 980 |
| 833623 | N/A | N/A | 5896 | 5911 | CATGATGCAGCCCGGG | 90 | 981 |
| 833639 | N/A | N/A | 6463 | 6478 | GGATTTTACGATCCAT | 97 | 982 |
| 833655 | N/A | N/A | 6832 | 6847 | TGGCAGACCCGGCATC | 86 | 983 |
| 833671 | N/A | N/A | 7111 | 7126 | AGGAACACATTGCATC | 99 | 984 |
| 833687 | N/A | N/A | 7701 | 7716 | AACTAGCTGGACCCAA | 71 | 985 |
| 833703 | N/A | N/A | 8425 | 8440 | CCGGGAATGGAGTCAC | 95 | 986 |
| 833719 | N/A | N/A | 8846 | 8861 | CTCGAGTTGATCTCTT | 60 | 987 |
| 833735 | N/A | N/A | 9346 | 9361 | AGGGATTGACATAGTG | 65 | 988 |
| 833751 | N/A | N/A | 9912 | 9927 | GAGAACGGCACTGTGA | 110 | 989 |
| 833767 | N/A | N/A | 10272 | 10287 | AGAGAGGTAAATCCCC | 48 | 990 |
| 833782 | N/A | N/A | 11392 | 11407 | AGCCTAGGTAGAATTT | 88 | 991 |
| 833798 | N/A | N/A | 11640 | 11655 | ACATTTATGGTGCCCT | 57 | 992 |
| 833814 | N/A | N/A | 12009 | 12024 | ACCAACAGATATACGC | 53 | 993 |
| 833830 | N/A | N/A | 12624 | 12639 | CCCTTAGCAACTCAGC | 57 | 994 |
| 833846 | N/A | N/A | 13020 | 13035 | CCTAAAGGTGGCTATC | 69 | 995 |
| 833862 | N/A | N/A | 13294 | 13309 | CTAGAAATAGATCGCT | 57 | 996 |
| 833878 | N/A | N/A | 14002 | 14017 | CCATCTTACGGAAGCA | 72 | 997 |
| 833894 | N/A | N/A | 14526 | 14541 | AGGTAGGGATGTGAGC | 73 | 998 |
| 833910 | N/A | N/A | 15387 | 15402 | TGCTTTTCGGCCCAGA | 33 | 999 |
| 833926 | N/A | N/A | 15779 | 15794 | AGGGATGCATGAAGAC | 98 | 1000 |
| 833942 | N/A | N/A | 16370 | 16385 | TTAGAACCCCACCATT | 82 | 1001 |
| 833958 | N/A | N/A | 17085 | 17100 | TAGCAGAGGGCAACCT | 78 | 1002 |
| 833974 | N/A | N/A | 17638 | 17653 | GGGCAATACCCAAGAC | 54 | 1003 |
| 833990 | N/A | N/A | 18538 | 18553 | CTTCATTGGCAGCCAC | 90 | 1004 |
| 834006* | N/A | N/A | 19183 | 19198 | ACCTAATGCAAAGTCC | 75 | 1005 |
| 834022* | N/A | N/A | 19473 | 19488 | ACGCAGACCACCAGGT | 122 | 1006 |

TABLE 14
Reduction of SPDEF RNA by 4 μM 3-10-3 cEt
gapmers with a phosphorothioate backbone

| | SEQ ID | SEQ ID | SEQ ID | SEQ ID | | | |
|---|---|---|---|---|---|---|---|
| | NO: 1 | NO: 1 | NO: 2 | NO: 2 | | | |
| Compound | Start | Stop | Start | Stop | | SPDEF | SEQ |
| Number | Site | Site | Site | Site | Sequence (5′ to 3′) | (% UTC) | ID NO |
| 791842 | 1061 | 1076 | N/A | N/A | CCAGGCCGCTGACTTC | 92 | 1007 |
| 791903 | 1361 | 1376 | 19646 | 19661 | CTTGTAATACTGGCGG | 87 | 1008 |
| 801766 | 712 | 727 | 13823 | 13838 | CTGTCAATGACCGGGC | 56 | 33 |
| 802095 | N/A | N/A | 17525 | 17540 | TTCATAGACTTTCCCT | 51 | 240 |
| 832830 | 44 | 59 | 1711 | 1726 | ACACGGCAGAGTGCAG | 103 | 1009 |
| 832846 | 123 | 138 | 1790 | 1805 | ACCGTGGCAAGGCCCA | 72 | 1010 |
| 832862 | 197 | 212 | 1864 | 1879 | GGTGGCCCTCTGAGGT | 113 | 1011 |
| 832878 | 397 | 412 | N/A | N/A | GCTGTGTCTGTTAGCT | 97 | 1012 |
| 832894 | 423 | 438 | 13534 | 13549 | CCATGCCGCTGCTGTT | 74 | 1013 |
| 832910 | 497 | 512 | 13608 | 13623 | TGTCCGCGACACCGTG | 86 | 1014 |
| 832926 | 546 | 561 | 13657 | 13672 | AGTCCCGTCTCTCGAG | 90 | 1015 |
| 832942 | 589 | 604 | 13700 | 13715 | GCGGACAGGCCCTGCT | 107 | 1016 |
| 832958 | 630 | 645 | 13741 | 13756 | TGTCCTCAGGGTACAG | 103 | 1017 |
| 832974 | 713 | 728 | 13824 | 13839 | GCTGTCAATGACCGGG | 67 | 1018 |
| 832990 | 775 | 790 | 13886 | 13901 | AGCGAGTGCTCCTCCA | 69 | 1019 |
| 833006 | 820 | 835 | 13931 | 13946 | ATGTCCTTGAGCACTT | 77 | 1020 |
| 833021 | 843 | 858 | 13954 | 13969 | TGAGCAGCTTGCAGGC | 70 | 1021 |
| 833037 | 920 | 935 | 16869 | 16884 | CCGGTATTGGTGCTCT | 104 | 1022 |
| 833051 | 986 | 1001 | 16935 | 16950 | CTCCTCCGACATGGCG | 100 | 1023 |
| 833079 | 1090 | 1105 | 18460 | 18475 | ATCGCCCCAGGTGAAG | 96 | 1024 |
| 833095 | 1114 | 1129 | N/A | N/A | TCACTGGTCGAGGCAC | 97 | 1025 |
| 833111 | 1148 | 1163 | 18636 | 18651 | TGATGAGTCCACCTCG | 80 | 1026 |
| 833125 | 1197 | 1212 | 18685 | 18700 | GTAGCAACTCCTTGAG | 69 | 1027 |
| 833139* | 1236 | 1251 | 18724 | 18739 | TGAGCCACCTAATGAA | 61 | 1028 |
| 833160 | 1453 | 1468 | 19738 | 19753 | CGGGTTTCAGGCCCTG | 85 | 1029 |
| 833176 | 1599 | 1614 | 19884 | 19899 | CAGAGGACCCATATCC | 82 | 1030 |
| 833192 | 1751 | 1766 | 20036 | 20051 | CCTTTGTCGAGTCACT | 68 | 1031 |
| 833224 | N/A | N/A | 5015 | 5030 | CCCCGCAACCAGTCTC | 86 | 1032 |
| 833240 | N/A | N/A | 5052 | 5067 | AACTTGCTACCCCAGG | 47 | 1033 |
| 833256 | N/A | N/A | 5144 | 5159 | TGTGAAGTGTCAGCAG | 89 | 1034 |
| 833272 | N/A | N/A | 5184 | 5199 | GGAGTGGGTTATTAAG | 90 | 1035 |
| 833288 | N/A | N/A | 5276 | 5291 | AGGACTCCAACATCAC | 62 | 1036 |
| 833304 | N/A | N/A | 5342 | 5357 | TGCGCCCTGATCCTCA | 75 | 1037 |
| 833320 | N/A | N/A | 5375 | 5390 | AAATCTTGATGGGCTG | 105 | 1038 |
| 833336 | N/A | N/A | 5451 | 5466 | GTTTCTGCGGCCCCTC | 54 | 1039 |
| 833352 | N/A | N/A | 5489 | 5504 | GTGTGTCCACAGTGGT | 65 | 1040 |
| 833368 | N/A | N/A | 5515 | 5530 | GAAGGCTTCACTGTCT | 86 | 1041 |
| 833384 | N/A | N/A | 5546 | 5561 | TTTGAAGTCACCAGGC | 88 | 1042 |
| 833400 | N/A | N/A | 5621 | 5636 | CCTTGGCCGATCCTCT | 78 | 1043 |
| 833416 | N/A | N/A | 5656 | 5671 | ATGTCACCGGAGCTCT | 58 | 1044 |
| 833432 | N/A | N/A | 5678 | 5693 | TTGAGGGCATGAGAAC | 112 | 1045 |
| 833448 | N/A | N/A | 4828 | 4843 | GAAGGATGAGCCTCTC | 82 | 1046 |
| 833464 | N/A | N/A | 4861 | 4876 | AGCAGAGTCACACTAA | 87 | 1047 |
| 833480 | N/A | N/A | 4886 | 4901 | GAGGTCCCGGTCCCAT | 74 | 1048 |
| 833496 | N/A | N/A | 4917 | 4932 | TATCCTGGTGGTGCGC | 81 | 1049 |
| 833512 | N/A | N/A | 18315 | 18330 | GGTTGCCCCTGTGGCT | 99 | 1050 |
| 833528 | N/A | N/A | 2530 | 2545 | TTAGACTTAGCCCTGA | 75 | 1051 |
| 833544 | N/A | N/A | 2918 | 2933 | ATGCACATGCAACACG | 94 | 1052 |
| 833560 | N/A | N/A | 3526 | 3541 | AGCAAGTCTGGTAGTT | 64 | 1053 |
| 833576 | N/A | N/A | 3752 | 3767 | TCTAACGGTTTCAGGG | 79 | 1054 |
| 833592 | N/A | N/A | 3965 | 3980 | TGCTTTAACAGGCAAT | 89 | 1055 |
| 833608 | N/A | N/A | 4660 | 4675 | GGGCAACTCGGCTTGT | 70 | 1056 |
| 833624 | N/A | N/A | 5904 | 5919 | CGCTTTGCCATGATGC | 92 | 1057 |
| 833640 | N/A | N/A | 6466 | 6481 | CCTGGATTTTACGATC | 79 | 1058 |
| 833656 | N/A | N/A | 6866 | 6881 | CAAGACTCGGCTCCAC | 66 | 1059 |
| 833672 | N/A | N/A | 7174 | 7189 | AGCCAAAGTGGAGCGC | 76 | 1060 |
| 833688 | N/A | N/A | 7703 | 7718 | CAAACTAGCTGGACCC | 80 | 1061 |
| 833704 | N/A | N/A | 8434 | 8449 | CCACAGTTTCCGGGAA | 88 | 1062 |
| 833720 | N/A | N/A | 8853 | 8868 | CCCAAACCTCGAGTTG | 67 | 1063 |
| 833736 | N/A | N/A | 9350 | 9365 | AACCAGGGATTGACAT | 91 | 1064 |
| 833752 | N/A | N/A | 9914 | 9929 | GAGAGAACGGCACTGT | 86 | 1065 |
| 833768 | N/A | N/A | 10303 | 10318 | CCCTACTTTGCTAATG | 101 | 1066 |
| 833783 | N/A | N/A | 11400 | 11415 | ACGAATGGAGCCTAGG | 72 | 1067 |
| 833799 | N/A | N/A | 11642 | 11657 | TGACATTTATGGTGCC | 59 | 1068 |
| 833815 | N/A | N/A | 12019 | 12034 | GCAGAAGATTACCAAC | 78 | 1069 |
| 833831 | N/A | N/A | 12632 | 12647 | ACCTAAAACCCTTAGC | 94 | 1070 |
| 833847 | N/A | N/A | 13026 | 13041 | CACCATCCTAAAGGTG | 83 | 1071 |
| 833863 | N/A | N/A | 13318 | 13333 | AGCGAGGTGGGAGTGG | 108 | 1072 |
| 833879 | N/A | N/A | 14004 | 14019 | ACCCATCTTACGGAAG | 72 | 1073 |
| 833895 | N/A | N/A | 14530 | 14545 | CAGCAGGTAGGGATGT | 96 | 1074 |
| 833911 | N/A | N/A | 15393 | 15408 | AGGAACTGCTTTTCGG | 46 | 1075 |
| 833927 | N/A | N/A | 15784 | 15799 | GCCTGAGGGATGCATG | 84 | 1076 |
| 833943 | N/A | N/A | 16372 | 16387 | TCTTAGAACCCCACCA | 100 | 1077 |
| 833959 | N/A | N/A | 17174 | 17189 | GTGAAGAGTGCACCAG | 74 | 1078 |
| 833975 | N/A | N/A | 17652 | 17667 | GTGGACACGGACAGGG | 64 | 1079 |
| 833991* | N/A | N/A | 18817 | 18832 | CCCCATGCACCGTGCC | 104 | 1080 |
| 834007* | N/A | N/A | 19254 | 19269 | CCTTAGTGGGTTCCCT | 94 | 1081 |
| 834023* | N/A | N/A | 19477 | 19492 | AAAGACGCAGACCACC | 98 | 1082 |

TABLE 15
Reduction of SPDEF RNA by 4 μM 3-10-3 cEt
gapmers with a phosphorothioate backbone

| | SEQ ID | SEQ ID | SEQ ID | SEQ ID | | | |
|---|---|---|---|---|---|---|---|
| | NO: 1 | NO: 1 | NO: 2 | NO: 2 | | | |
| Compound | Start | Stop | Start | Stop | | SPDEF | SEQ |
| Number | Site | Site | Site | Site | Sequence (5′ to 3′) | (% UTC) | ID NO |
| 791905 | 1364 | 1379 | 19649 | 19664 | CTTCTTGTAATACTGG | 85 | 1083 |
| 801766 | 712 | 727 | 13823 | 13838 | CTGTCAATGACCGGGC | 58 | 33 |
| 802095 | N/A | N/A | 17525 | 17540 | TTCATAGACTTTCCCT | 44 | 240 |
| 832831 | 45 | 60 | 1712 | 1727 | GACACGGCAGAGTGCA | 98 | 1084 |
| 832847 | 125 | 140 | 1792 | 1807 | GCACCGTGGCAAGGCC | 108 | 1085 |
| 832863 | 198 | 213 | 1865 | 1880 | GGGTGGCCCTCTGAGG | 99 | 1086 |
| 832879 | 399 | 414 | N/A | N/A | CGGCTGTGTCTGTTAG | 112 | 1087 |
| 832895 | 424 | 439 | 13535 | 13550 | CCCATGCCGCTGCTGT | 68 | 1088 |
| 832911 | 498 | 513 | 13609 | 13624 | CTGTCCGCGACACCGT | 56 | 1089 |
| 832927 | 547 | 562 | 13658 | 13673 | CAGTCCCGTCTCTCGA | 90 | 1090 |
| 832943 | 591 | 606 | 13702 | 13717 | AGGCGGACAGGCCCTG | 99 | 1091 |
| 832959 | 631 | 646 | 13742 | 13757 | CTGTCCTCAGGGTACA | 72 | 1092 |
| 832975 | 715 | 730 | 13826 | 13841 | TGGCTGTCAATGACCG | 89 | 1093 |
| 832991 | 778 | 793 | 13889 | 13904 | TCCAGCGAGTGCTCCT | 81 | 1094 |
| 833007 | 821 | 836 | 13932 | 13947 | GATGTCCTTGAGCACT | 101 | 1095 |
| 833022 | 844 | 859 | 13955 | 13970 | TTGAGCAGCTTGCAGG | 99 | 1096 |
| 833038 | 922 | 937 | 16871 | 16886 | AGCCGGTATTGGTGCT | 127 | 1097 |
| 833052 | 989 | 1004 | 16938 | 16953 | CTGCTCCTCCGACATG | 111 | 1098 |
| 833065 | 1062 | 1077 | N/A | N/A | TCCAGGCCGCTGACTT | 117 | 1099 |
| 833080 | 1091 | 1106 | 18461 | 18476 | AATCGCCCCAGGTGAA | 111 | 1100 |
| 833096 | 1115 | 1130 | N/A | N/A | CTCACTGGTCGAGGCA | 119 | 1101 |
| 833112 | 1149 | 1164 | 18637 | 18652 | ATGATGAGTCCACCTC | 79 | 1102 |
| 833126 | 1198 | 1213 | 18686 | 18701 | AGTAGCAACTCCTTGA | 76 | 1103 |
| 833140* | 1237 | 1252 | 18725 | 18740 | TTGAGCCACCTAATGA | 24* | 1104 |
| 833161 | 1454 | 1469 | 19739 | 19754 | GCGGGTTTCAGGCCCT | 114 | 1105 |
| 833177 | 1600 | 1615 | 19885 | 19900 | CCAGAGGACCCATATC | 109 | 1106 |
| 833193 | 1752 | 1767 | 20037 | 20052 | GCCTTTGTCGAGTCAC | 74 | 1107 |
| 833225 | N/A | N/A | 5016 | 5031 | GCCCCGCAACCAGTCT | 103 | 1108 |
| 833241 | N/A | N/A | 5053 | 5068 | CAACTTGCTACCCCAG | 60 | 1109 |
| 833257 | N/A | N/A | 5146 | 5161 | CCTGTGAAGTGTCAGC | 85 | 1110 |
| 833273 | N/A | N/A | 5203 | 5218 | AACAAGGTTGAGATGG | 75 | 1111 |
| 833289 | N/A | N/A | 5277 | 5292 | AAGGACTCCAACATCA | 100 | 1112 |
| 833305 | N/A | N/A | 5343 | 5358 | CTGCGCCCTGATCCTC | 75 | 1113 |
| 833321 | N/A | N/A | 5376 | 5391 | AAAATCTTGATGGGCT | 70 | 1114 |
| 833337 | N/A | N/A | 5452 | 5467 | TGTTTCTGCGGCCCCT | 76 | 1115 |
| 833353 | N/A | N/A | 5494 | 5509 | GAGGTGTGTGTCCACA | 64 | 1116 |
| 833369 | N/A | N/A | 5521 | 5536 | ATGTGAGAAGGCTTCA | 114 | 1117 |
| 833385 | N/A | N/A | 5577 | 5592 | GGGACTCATAAAGACA | 103 | 1118 |
| 833401 | N/A | N/A | 5622 | 5637 | CCCTTGGCCGATCCTC | 55 | 1119 |
| 833417 | N/A | N/A | 5657 | 5672 | GATGTCACCGGAGCTC | 78 | 1120 |
| 833433 | N/A | N/A | 5679 | 5694 | GTTGAGGGCATGAGAA | 143 | 1121 |
| 833449 | N/A | N/A | 4829 | 4844 | GGAAGGATGAGCCTCT | 120 | 1122 |
| 833465 | N/A | N/A | 4862 | 4877 | CAGCAGAGTCACACTA | 102 | 1123 |
| 833481 | N/A | N/A | 4887 | 4902 | GGAGGTCCCGGTCCCA | 96 | 1124 |
| 833497 | N/A | N/A | 4918 | 4933 | TTATCCTGGTGGTGCG | 114 | 1125 |
| 833513 | N/A | N/A | 18316 | 18331 | GGGTTGCCCCTGTGGC | 92 | 1126 |
| 833529 | N/A | N/A | 2533 | 2548 | GCCTTAGACTTAGCCC | 90 | 1127 |
| 833545 | N/A | N/A | 2983 | 2998 | GCTGATAGGTGAGGTG | 103 | 1128 |
| 833561 | N/A | N/A | 3531 | 3546 | CAATAAGCAAGTCTGG | 46 | 1129 |
| 833577 | N/A | N/A | 3754 | 3769 | GCTCTAACGGTTTCAG | 82 | 1130 |
| 833593 | N/A | N/A | 3974 | 3989 | CGTGAAGCCTGCTTTA | 117 | 1131 |
| 833609 | N/A | N/A | 4666 | 4681 | AGCCCAGGGCAACTCG | 56 | 1132 |
| 833625 | N/A | N/A | 5907 | 5922 | CCCCGCTTTGCCATGA | 106 | 1133 |
| 833641 | N/A | N/A | 6511 | 6526 | CTGTAGGCCAGGTCAT | 113 | 1134 |
| 833657 | N/A | N/A | 6868 | 6883 | CTCAAGACTCGGCTCC | 68 | 1135 |
| 833673 | N/A | N/A | 7298 | 7313 | AGCTAGTGGGCCCAGG | 92 | 1136 |
| 833689 | N/A | N/A | 7706 | 7721 | CTTCAAACTAGCTGGA | 93 | 1137 |
| 833705 | N/A | N/A | 8436 | 8451 | AACCACAGTTTCCGGG | 70 | 1138 |
| 833721 | N/A | N/A | 8873 | 8888 | CCTGAGCGATGCCTCC | 85 | 1139 |
| 833737 | N/A | N/A | 9354 | 9369 | CCAGAACCAGGGATTG | 74 | 1140 |
| 833753 | N/A | N/A | 9917 | 9932 | GAAGAGAGAACGGCAC | 80 | 1141 |
| 833769 | N/A | N/A | 10342 | 10357 | GGCACAAGCTACCTCA | 75 | 1142 |
| 833784 | N/A | N/A | 11406 | 11421 | AGCTTGACGAATGGAG | 108 | 1143 |
| 833800 | N/A | N/A | 11653 | 11668 | CTCTCTAACAGTGACA | 91 | 1144 |
| 833816 | N/A | N/A | 12371 | 12386 | ATACATCAAGACAGGC | 62 | 1145 |
| 833832 | N/A | N/A | 12636 | 12651 | GAACACCTAAAACCCT | 74 | 1146 |
| 833848 | N/A | N/A | 13055 | 13070 | GGATAGGAGTGGAAGT | 96 | 1147 |
| 833864 | N/A | N/A | 13324 | 13339 | AAAGACAGCGAGGTGG | 80 | 1148 |
| 833880 | N/A | N/A | 14026 | 14041 | AGCGACCTCAGCCTTG | 86 | 1149 |
| 833896 | N/A | N/A | 14567 | 14582 | GAGGAGTGTAAGTGCT | 83 | 1150 |
| 833912 | N/A | N/A | 15400 | 15415 | GCATATTAGGAACTGC | 77 | 1151 |
| 833928 | N/A | N/A | 15863 | 15878 | GCATTGGGAAACTTGG | 79 | 1152 |
| 833944 | N/A | N/A | 16469 | 16484 | TGACACTCTACCAGAA | 82 | 1153 |
| 833960 | N/A | N/A | 17297 | 17312 | GTATCCACGGTTGTCC | 78 | 1154 |
| 833976 | N/A | N/A | 17767 | 17782 | GAAACAGGGAAGTCGA | 78 | 1155 |
| 833992* | N/A | N/A | 18864 | 18879 | TCTAGGACAAAGGTGG | 102 | 1156 |
| 834008* | N/A | N/A | 19256 | 19271 | TACCTTAGTGGGTTCC | 88 | 1157 |
| 834024* | N/A | N/A | 19480 | 19495 | GAGAAAGACGCAGACC | 127 | 1158 |

TABLE 16
Reduction of SPDEF RNA by 4 μM 3-10-3 cEt
gapmers with a phosphorothioate backbone

| | SEQ ID | SEQ ID | SEQ ID | SEQ ID | | | |
|---|---|---|---|---|---|---|---|
| | NO: 1 | NO: 1 | NO: 2 | NO: 2 | | | |
| Compound | Start | Stop | Start | Stop | | SPDEF | SEQ |
| Number | Site | Site | Site | Site | Sequence (5′ to 3′) | (% UTC) | ID NO |
| 652523 | 1365 | 1380 | 19650 | 19665 | CCTTCTTGTAATACTG | 95 | 1159 |
| 801766 | 712 | 727 | 13823 | 13838 | CTGTCAATGACCGGGC | 63 | 33 |
| 802095 | N/A | N/A | 17525 | 17540 | TTCATAGACTTTCCCT | 50 | 240 |
| 832832 | 47 | 62 | 1714 | 1729 | TGGACACGGCAGAGTG | 73 | 1160 |
| 832848 | 127 | 142 | 1794 | 1809 | TGGCACCGTGGCAAGG | 83 | 1161 |
| 832864 | 212 | 227 | 1879 | 1894 | TGGCCACCCTCAAGGG | 100 | 1162 |
| 832880 | 400 | 415 | N/A | N/A | GCGGCTGTGTCTGTTA | 110 | 1163 |
| 832896 | 426 | 441 | 13537 | 13552 | TGCCCATGCCGCTGCT | 79 | 1164 |
| 832912 | 499 | 514 | 13610 | 13625 | CCTGTCCGCGACACCG | 89 | 1165 |
| 832928 | 548 | 563 | 13659 | 13674 | CCAGTCCCGTCTCTCG | 98 | 1166 |
| 832944 | 592 | 607 | 13703 | 13718 | AAGGCGGACAGGCCCT | 96 | 1167 |
| 832960 | 668 | 683 | 13779 | 13794 | CCGACTGCTGGCCCCA | 98 | 1168 |
| 832976 | 718 | 733 | 13829 | 13844 | GCTTGGCTGTCAATGA | 90 | 1169 |
| 832992 | 783 | 798 | 13894 | 13909 | CCTGCTCCAGCGAGTG | 109 | 1170 |
| 833008 | 822 | 837 | 13933 | 13948 | CGATGTCCTTGAGCAC | 72 | 1171 |
| 833023 | 845 | 860 | 13956 | 13971 | GTTGAGCAGCTTGCAG | 108 | 1172 |
| 833039 | 924 | 939 | 16873 | 16888 | GCAGCCGGTATTGGTG | 116 | 1173 |
| 833053 | 990 | 1005 | 16939 | 16954 | ACTGCTCCTCCGACAT | 113 | 1174 |
| 833066 | 1064 | 1079 | N/A | N/A | CATCCAGGCCGCTGAC | 111 | 1175 |
| 833081 | 1093 | 1108 | 18463 | 18478 | TGAATCGCCCCAGGTG | 85 | 1176 |
| 833097 | 1117 | 1132 | 18605 | 18620 | TCCTCACTGGTCGAGG | 92 | 1177 |
| 833113 | 1150 | 1165 | 18638 | 18653 | CATGATGAGTCCACCT | 132 | 1178 |
| 833127 | 1200 | 1215 | 18688 | 18703 | TGAGTAGCAACTCCTT | 117 | 1179 |
| 833141* | 1239 | 1254 | 18727 | 18742 | TGTTGAGCCACCTAAT | 76 | 1180 |
| 833162 | 1516 | 1531 | 19801 | 19816 | CCCGTTTTCCCCCATC | 87 | 1181 |
| 833178 | 1611 | 1626 | 19896 | 19911 | TCCCGAAGGCCCCAGA | 92 | 1182 |
| 833194 | 1754 | 1769 | 20039 | 20054 | TGGCCTTTGTCGAGTC | 86 | 1183 |
| 833210 | N/A | N/A | 18602 | 18617 | TCACTGGTCGAGGCTG | 141 | 1184 |
| 833226 | N/A | N/A | 5017 | 5032 | TGCCCCGCAACCAGTC | 89 | 1185 |
| 833242 | N/A | N/A | 5054 | 5069 | GCAACTTGCTACCCCA | 46 | 1186 |
| 833258 | N/A | N/A | 5147 | 5162 | ACCTGTGAAGTGTCAG | 87 | 1187 |
| 833274 | N/A | N/A | 5207 | 5222 | TTCAAACAAGGTTGAG | 95 | 1188 |
| 833290 | N/A | N/A | 5279 | 5294 | TGAAGGACTCCAACAT | 107 | 1189 |
| 833306 | N/A | N/A | 5344 | 5359 | CCTGCGCCCTGATCCT | 90 | 1190 |
| 833322 | N/A | N/A | 5377 | 5392 | TAAAATCTTGATGGGC | 98 | 1191 |
| 833338 | N/A | N/A | 5453 | 5468 | GTGTTTCTGCGGCCCC | 61 | 1192 |
| 833354 | N/A | N/A | 5495 | 5510 | AGAGGTGTGTGTCCAC | 67 | 1193 |
| 833370 | N/A | N/A | 5522 | 5537 | GATGTGAGAAGGCTTC | 69 | 1194 |
| 833386 | N/A | N/A | 5578 | 5593 | AGGGACTCATAAAGAC | 112 | 1195 |
| 833402 | N/A | N/A | 5623 | 5638 | CCCCTTGGCCGATCCT | 81 | 1196 |
| 833418 | N/A | N/A | 5658 | 5673 | GGATGTCACCGGAGCT | 76 | 1197 |
| 833434 | N/A | N/A | 5680 | 5695 | CGTTGAGGGCATGAGA | 133 | 1198 |
| 833450 | N/A | N/A | 4830 | 4845 | AGGAAGGATGAGCCTC | 99 | 1199 |
| 833466 | N/A | N/A | 4870 | 4885 | CCGACCCCCAGCAGAG | 109 | 1200 |
| 833482 | N/A | N/A | 4888 | 4903 | GGGAGGTCCCGGTCCC | 92 | 1201 |
| 833498 | N/A | N/A | 4919 | 4934 | TTTATCCTGGTGGTGC | 73 | 1202 |
| 833530 | N/A | N/A | 2548 | 2563 | CCCGAACTGGACCCGG | 103 | 1203 |
| 833546 | N/A | N/A | 3035 | 3050 | ACACAGGCTACGCGGG | 95 | 1204 |
| 833562 | N/A | N/A | 3584 | 3599 | TCGAATTCAGAGGGTC | 89 | 1205 |
| 833578 | N/A | N/A | 3783 | 3798 | GGCTGCAACAAGTCAT | 82 | 1206 |
| 833594 | N/A | N/A | 4028 | 4043 | TGGCAAATCCAACTCC | 105 | 1207 |
| 833610 | N/A | N/A | 4690 | 4705 | AGCTTGGCATTAAATG | 99 | 1208 |
| 833626 | N/A | N/A | 5913 | 5928 | GCACATCCCCGCTTTG | 90 | 1209 |
| 833642 | N/A | N/A | 6567 | 6582 | TCCATAGGAGAGACCC | 89 | 1210 |
| 833658 | N/A | N/A | 6870 | 6885 | AACTCAAGACTCGGCT | 72 | 1211 |
| 833674 | N/A | N/A | 7302 | 7317 | AGCCAGCTAGTGGGCC | 92 | 1212 |
| 833690 | N/A | N/A | 7870 | 7885 | CCGCAGTAGCATGTCT | 80 | 1213 |
| 833706 | N/A | N/A | 8439 | 8454 | CCGAACCACAGTTTCC | 68 | 1214 |
| 833722 | N/A | N/A | 8889 | 8904 | CCACGGGCTGCCGTCT | 83 | 1215 |
| 833738 | N/A | N/A | 9356 | 9371 | GACCAGAACCAGGGAT | 61 | 1216 |
| 833754 | N/A | N/A | 9919 | 9934 | AAGAAGAGAGAACGGC | 86 | 1217 |
| 833770 | N/A | N/A | 10619 | 10634 | AGCACAGGCCTTACTC | 117 | 1218 |
| 833785 | N/A | N/A | 11410 | 11425 | GCATAGCTTGACGAAT | 74 | 1219 |
| 833801 | N/A | N/A | 11705 | 11720 | TTAAAGGTAACTGGCC | 90 | 1220 |
| 833817 | N/A | N/A | 12373 | 12388 | GAATACATCAAGACAG | 79 | 1221 |
| 833833 | N/A | N/A | 12638 | 12653 | GGGAACACCTAAAACC | 96 | 1222 |
| 833849 | N/A | N/A | 13058 | 13073 | CAAGGATAGGAGTGGA | 38 | 1223 |
| 833865 | N/A | N/A | 13326 | 13341 | GGAAAGACAGCGAGGT | 81 | 1224 |
| 833881 | N/A | N/A | 14091 | 14106 | CCAAAGCTGCCCGAGG | 115 | 1225 |
| 833897 | N/A | N/A | 14571 | 14586 | GTCCGAGGAGTGTAAG | 95 | 1226 |
| 833913 | N/A | N/A | 15465 | 15480 | GCCCTACGAACACAGG | 90 | 1227 |
| 833929 | N/A | N/A | 15884 | 15899 | CCTGGAGTCGGCCTGG | 105 | 1228 |
| 833945 | N/A | N/A | 16499 | 16514 | TTCGAGGGAGCCTCAG | 89 | 1229 |
| 833961 | N/A | N/A | 17302 | 17317 | TCCTAGTATCCACGGT | 67 | 1230 |
| 833977 | N/A | N/A | 17996 | 18011 | TGACACGCAGCCATTA | 127 | 1231 |
| 833993* | N/A | N/A | 18874 | 18889 | ATATTTGGCATCTAGG | 120 | 1232 |
| 834009* | N/A | N/A | 19273 | 19288 | GTACAGGTGAGCCTGT | 96 | 1233 |
| 834025* | N/A | N/A | 19502 | 19517 | GTATGAGTGAGGTGGC | 115 | 1234 |

TABLE 17
Reduction of SPDEF RNA by 4 μM 3-10-3 cEt
gapmers with a phosphorothioate backbone

| | SEQ ID | SEQ ID | SEQ ID | SEQ ID | | | |
|---|---|---|---|---|---|---|---|
| | NO: 1 | NO: 1 | NO: 2 | NO: 2 | | | |
| Compound | Start | Stop | Start | Stop | | SPDEF | SEQ |
| Number | Site | Site | Site | Site | Sequence (5′ to 3′) | (% UTC) | ID NO |
| 652482 | 1066 | 1081 | 18436 | 18451 | TTCATCCAGGCCGCTG | 97 | 1235 |
| 791906 | 1366 | 1381 | 19651 | 19666 | CCCTTCTTGTAATACT | 87 | 1236 |
| 801766 | 712 | 727 | 13823 | 13838 | CTGTCAATGACCGGGC | 57 | 33 |
| 802095 | N/A | N/A | 17525 | 17540 | TTCATAGACTTTCCCT | 41 | 240 |
| 832833 | 49 | 64 | 1716 | 1731 | TGTGGACACGGCAGAG | 86 | 1237 |
| 832849 | 128 | 143 | 1795 | 1810 | CTGGCACCGTGGCAAG | 112 | 1238 |
| 832865 | 213 | 228 | 1880 | 1895 | CTGGCCACCCTCAAGG | 99 | 1239 |
| 832881 | 402 | 417 | N/A | N/A | TGGCGGCTGTGTCTGT | 15 | 1240 |
| 832897 | 427 | 442 | 13538 | 13553 | CTGCCCATGCCGCTGC | 77 | 1241 |
| 832913 | 501 | 516 | 13612 | 13627 | AGCCTGTCCGCGACAC | 70 | 1242 |
| 832929 | 549 | 564 | 13660 | 13675 | TCCAGTCCCGTCTCTC | 76 | 1243 |
| 832945 | 594 | 609 | 13705 | 13720 | AGAAGGCGGACAGGCC | 95 | 1244 |
| 832961 | 670 | 685 | 13781 | 13796 | TCCCGACTGCTGGCCC | 91 | 1245 |
| 832977 | 720 | 735 | 13831 | 13846 | GGGCTTGGCTGTCAAT | 83 | 1246 |
| 832993 | 785 | 800 | 13896 | 13911 | CACCTGCTCCAGCGAG | 74 | 1247 |
| 833009 | 823 | 838 | 13934 | 13949 | TCGATGTCCTTGAGCA | 77 | 1248 |
| 833024 | 855 | 870 | 13966 | 13981 | CTGCGGTGATGTTGAG | 100 | 1249 |
| 833040 | 925 | 940 | 16874 | 16889 | GGCAGCCGGTATTGGT | 94 | 1250 |
| 833054 | 992 | 1007 | 16941 | 16956 | GAACTGCTCCTCCGAC | 81 | 1251 |
| 833082 | 1095 | 1110 | 18465 | 18480 | AGTGAATCGCCCCAGG | 59 | 1252 |
| 833098 | 1118 | 1133 | 18606 | 18621 | CTCCTCACTGGTCGAG | 105 | 1253 |
| 833114 | 1152 | 1167 | 18640 | 18655 | AGCATGATGAGTCCAC | 75 | 1254 |
| 833128 | 1201 | 1216 | 18689 | 18704 | TTGAGTAGCAACTCCT | 73 | 1255 |
| 833142* | 1240 | 1255 | 18728 | 18743 | TTGTTGAGCCACCTAA | 98 | 1256 |
| 833163 | 1517 | 1532 | 19802 | 19817 | GCCCGTTTTCCCCCAT | 61 | 1257 |
| 833179 | 1612 | 1627 | 19897 | 19912 | GTCCCGAAGGCCCCAG | 88 | 1258 |
| 833195 | 1756 | 1771 | 20041 | 20056 | TGTGGCCTTTGTCGAG | 95 | 1259 |
| 833211 | N/A | N/A | 4922 | 4937 | GTCTTTATCCTGGTGG | 59 | 1260 |
| 833227 | N/A | N/A | 5018 | 5033 | TTGCCCCGCAACCAGT | 74 | 1261 |
| 833243 | N/A | N/A | 5055 | 5070 | AGCAACTTGCTACCCC | 48 | 1262 |
| 833259 | N/A | N/A | 5148 | 5163 | CACCTGTGAAGTGTCA | 100 | 1263 |
| 833275 | N/A | N/A | 5208 | 5223 | CTTCAAACAAGGTTGA | 80 | 1264 |
| 833291 | N/A | N/A | 5280 | 5295 | GTGAAGGACTCCAACA | 84 | 1265 |
| 833307 | N/A | N/A | 5345 | 5360 | TCCTGCGCCCTGATCC | 83 | 1266 |
| 833323 | N/A | N/A | 5384 | 5399 | TGGTCTGTAAAATCTT | 98 | 1267 |
| 833339 | N/A | N/A | 5454 | 5469 | CGTGTTTCTGCGGCCC | 69 | 1268 |
| 833355 | N/A | N/A | 5496 | 5511 | GAGAGGTGTGTGTCCA | 112 | 1269 |
| 833371 | N/A | N/A | 5523 | 5538 | CGATGTGAGAAGGCTT | 124 | 1270 |
| 833387 | N/A | N/A | 5579 | 5594 | CAGGGACTCATAAAGA | 118 | 1271 |
| 833403 | N/A | N/A | 5625 | 5640 | GGCCCCTTGGCCGATC | 80 | 1272 |
| 833419 | N/A | N/A | 5659 | 5674 | CGGATGTCACCGGAGC | 55 | 1273 |
| 833435 | N/A | N/A | 5716 | 5731 | GGGTCTCTTGCTCCCC | 101 | 1274 |
| 833451 | N/A | N/A | 4831 | 4846 | GAGGAAGGATGAGCCT | 100 | 1275 |
| 833467 | N/A | N/A | 4871 | 4886 | TCCGACCCCCAGCAGA | 74 | 1276 |
| 833483 | N/A | N/A | 4902 | 4917 | CCGTCATAATCCTGGG | 72 | 1277 |
| 833499 | N/A | N/A | 4920 | 4935 | CTTTATCCTGGTGGTG | 59 | 1278 |
| 833531 | N/A | N/A | 2631 | 2646 | GCTCAGCGGTGACCCC | 66 | 1279 |
| 833547 | N/A | N/A | 3047 | 3062 | CCATCATAAAGGACAC | 72 | 1280 |
| 833563 | N/A | N/A | 3589 | 3604 | CCCTATCGAATTCAGA | 115 | 1281 |
| 833579 | N/A | N/A | 3793 | 3808 | GTTATACTCAGGCTGC | 42 | 1282 |
| 833595 | N/A | N/A | 4040 | 4055 | CAGGAGACCGGCTGGC | 90 | 1283 |
| 833611 | N/A | N/A | 4755 | 4770 | GGGAGAGCAGAATCTG | 80 | 1284 |
| 833627 | N/A | N/A | 5915 | 5930 | CTGCACATCCCCGCTT | 113 | 1285 |
| 833643 | N/A | N/A | 6616 | 6631 | ACGAAGACCTCCACTT | 80 | 1286 |
| 833659 | N/A | N/A | 6874 | 6889 | CTCGAACTCAAGACTC | 61 | 1287 |
| 833675 | N/A | N/A | 7369 | 7384 | GTCCAGGCCAACTGTC | 73 | 1288 |
| 833691 | N/A | N/A | 7872 | 7887 | AGCCGCAGTAGCATGT | 86 | 1289 |
| 833707 | N/A | N/A | 8449 | 8464 | CTGACAGCTCCCGAAC | 81 | 1290 |
| 833723 | N/A | N/A | 8897 | 8912 | TGCCAGTCCCACGGGC | 94 | 1291 |
| 833739 | N/A | N/A | 9361 | 9376 | GTCCGGACCAGAACCA | 76 | 1292 |
| 833755 | N/A | N/A | 9969 | 9984 | GCCCAACCTGCAACTA | 77 | 1293 |
| 833771 | N/A | N/A | 10690 | 10705 | TGACACATCCTTGACA | 81 | 1294 |
| 833786 | N/A | N/A | 11412 | 11427 | TAGCATAGCTTGACGA | 82 | 1295 |
| 833802 | N/A | N/A | 11708 | 11723 | GCTTTAAAGGTAACTG | 68 | 1296 |
| 833818 | N/A | N/A | 12383 | 12398 | TGAGACTTAAGAATAC | 88 | 1297 |
| 833834 | N/A | N/A | 12708 | 12723 | CCGGAGGCAGTGCCAC | 90 | 1298 |
| 833850 | N/A | N/A | 13061 | 13076 | TGACAAGGATAGGAGT | 76 | 1299 |
| 833866 | N/A | N/A | 13344 | 13359 | AGACAGGCCTTCTGGC | 71 | 1300 |
| 833882 | N/A | N/A | 14111 | 14126 | GTGTAGAAGTGCCAGC | 56 | 1301 |
| 833898 | N/A | N/A | 14588 | 14603 | CAGATATGGTGCGGCA | 75 | 1302 |
| 833914 | N/A | N/A | 15470 | 15485 | TGTCAGCCCTACGAAC | 108 | 1303 |
| 833930 | N/A | N/A | 16005 | 16020 | CACTTAATAAGCCCAT | 85 | 1304 |
| 833946 | N/A | N/A | 16503 | 16518 | ACCTTTCGAGGGAGCC | 78 | 1305 |
| 833962 | N/A | N/A | 17419 | 17434 | TGGTACACTACTTTTC | 55 | 1306 |
| 833978 | N/A | N/A | 18014 | 18029 | ATTTAGACACTCAGGG | 69 | 1307 |
| 833994* | N/A | N/A | 18918 | 18933 | ACACAGATTGCACACA | 93 | 1308 |
| 834010* | N/A | N/A | 19275 | 19290 | CGGTACAGGTGAGCCT | 91 | 1309 |
| 834026* | N/A | N/A | 19510 | 19525 | AGCCAGTGGTATGAGT | 97 | 1310 |

TABLE 18
Reduction of SPDEF RNA by 4 μM 3-10-3 cEt
gapmers with a phosphorothioate backbone

| | SEQ | SEQ | SEQ | SEQ | | | |
|---|---|---|---|---|---|---|---|
| | ID | ID | ID | ID | | | |
| | NO: | NO: | NO: | NO: | | | |
| Com- | 1 | 1 | 2 | 2 | | SPDEF | SEQ |
| pound | Start | Stop | Start | Stop | Sequence | (% | ID |
| Number | Site | Site | Site | Site | (5′ to 3′) | UTC) | NO |
| 801766 | 712 | 727 | 13823 | 13838 | CTGTCAATGA | 56 | 33 |
| | | | | | CCGGGC | | |
| 802095 | N/A | N/A | 17525 | 17540 | TTCATAGACT | 47 | 240 |
| | | | | | TTCCCT | | |
| 832834 | 50 | 65 | 1717 | 1732 | GTGTGGACAC | 80 | 1311 |
| | | | | | GGCAGA | | |
| 832850 | 129 | 144 | 1796 | 1811 | GCTGGCACCG | 105 | 1312 |
| | | | | | TGGCAA | | |
| 832866 | 236 | 251 | 1903 | 1918 | CACTCAGGTT | 86 | 1313 |
| | | | | | GGCCAC | | |
| 832882 | 403 | 418 | N/A | N/A | CTGGCGGCTG | 97 | 1314 |
| | | | | | TGTCTG | | |
| 832898 | 442 | 457 | 13553 | 13568 | AGACCCGGGC | 87 | 1315 |
| | | | | | TGGCGC | | |
| 832914 | 503 | 518 | 13614 | 13629 | CAAGCCTGTC | 57 | 1316 |
| | | | | | CGCGAC | | |
| 832930 | 551 | 566 | 13662 | 13677 | ACTCCAGTCC | 107 | 1317 |
| | | | | | CGTCTC | | |
| 832946 | 596 | 611 | 13707 | 13722 | GTAGAAGGCG | 80 | 1318 |
| | | | | | GACAGG | | |
| 832962 | 672 | 687 | 13783 | 13798 | CCTCCCGACT | 80 | 1319 |
| | | | | | GCTGGC | | |
| 832978 | 750 | 765 | 13861 | 13876 | CGCCGGGCAC | 100 | 1320 |
| | | | | | CAAGTC | | |
| 832994 | 793 | 808 | 13904 | 13919 | ATGGACTGCA | 65 | 1321 |
| | | | | | CCTGCT | | |
| 833010 | 825 | 840 | 13936 | 13951 | TCTCGATGTC | 122 | 1322 |
| | | | | | CTTGAG | | |
| 833025 | 856 | 871 | N/A | N/A | TCTGCGGTGA | 69 | 1323 |
| | | | | | TGTTGA | | |
| 833041 | 942 | 957 | 16891 | 16906 | AGGCCTTGCC | 16 | 1324 |
| | | | | | CATGGG | | |
| 833055 | 993 | 1008 | 16942 | 16957 | GGAACTGCTC | 65 | 1325 |
| | | | | | CTCCGA | | |
| 833067 | 1067 | 1082 | 18437 | 18452 | TTTCATCCAG | 94 | 1326 |
| | | | | | GCCGCT | | |
| 833083 | 1096 | 1111 | 18466 | 18481 | TAGTGAATCG | 72 | 1327 |
| | | | | | CCCCAG | | |
| 833099 | 1119 | 1134 | 18607 | 18622 | TCTCCTCACT | 89 | 1328 |
| | | | | | GGTCGA | | |
| 833115 | 1153 | 1168 | 18641 | 18656 | GAGCATGATG | 84 | 1329 |
| | | | | | AGTCCA | | |
| 833129 | 1203 | 1218 | 18691 | 18706 | GCTTGAGTAG | 96 | 1330 |
| | | | | | CAACTC | | |
| 833143* | 1241 | 1256 | 18729 | 18744 | CTTGTTGAGC | 30 | 1331 |
| | | | | | CACCTA | | |
| 833148 | 1382 | 1397 | 19667 | 19682 | TGGCTTCCGG | 111 | 1332 |
| | | | | | ATGATG | | |
| 833164 | 1519 | 1534 | 19804 | 19819 | CTGCCCGTTT | 57 | 1333 |
| | | | | | TCCCCC | | |
| 833180 | 1613 | 1628 | 19898 | 19913 | GGTCCCGAAG | 88 | 1334 |
| | | | | | GCCCCA | | |
| 833196 | 1757 | 1772 | 20042 | 20057 | CTGTGGCCTT | 80 | 1335 |
| | | | | | TGTCGA | | |
| 833212 | N/A | N/A | 4924 | 4939 | GGGTCTTTAT | 68 | 1336 |
| | | | | | CCTGGT | | |
| 833228 | N/A | N/A | 5019 | 5034 | CTTGCCCCGC | 56 | 1337 |
| | | | | | AACCAG | | |
| 833244 | N/A | N/A | 5056 | 5071 | AAGCAACTTG | 67 | 1338 |
| | | | | | CTACCC | | |
| 833260 | N/A | N/A | 5157 | 5172 | ACCGGTTCCC | 90 | 1339 |
| | | | | | ACCTGT | | |
| 833276 | N/A | N/A | 5209 | 5224 | GCTTCAAACA | 47 | 1340 |
| | | | | | AGGTTG | | |
| 833292 | N/A | N/A | 5281 | 5296 | AGTGAAGGAC | 106 | 1341 |
| | | | | | TCCAAC | | |
| 833308 | N/A | N/A | 5346 | 5361 | TTCCTGCGCC | 70 | 1342 |
| | | | | | CTGATC | | |
| 833324 | N/A | N/A | 5389 | 5404 | GTTGTTGGTC | 115 | 1343 |
| | | | | | TGTAAA | | |
| 833340 | N/A | N/A | 5455 | 5470 | CCGTGTTTCT | 52 | 1344 |
| | | | | | GCGGCC | | |
| 833356 | N/A | N/A | 5497 | 5512 | CGAGAGGTGT | 79 | 1345 |
| | | | | | GTGTCC | | |
| 833372 | N/A | N/A | 5524 | 5539 | GCGATGTGAG | 83 | 1346 |
| | | | | | AAGGCT | | |
| 833388 | N/A | N/A | 5580 | 5595 | ACAGGGACTC | 124 | 1347 |
| | | | | | ATAAAG | | |
| 833404 | N/A | N/A | 5626 | 5641 | AGGCCCCTTG | 109 | 1348 |
| | | | | | GCCGAT | | |
| 833420 | N/A | N/A | 5660 | 5675 | ACGGATGTCA | 63 | 1349 |
| | | | | | CCGGAG | | |
| 833436 | N/A | N/A | 5717 | 5732 | TGGGTCTCTT | 91 | 1350 |
| | | | | | GCTCCC | | |
| 833452 | N/A | N/A | 4849 | 4864 | CTAAGGTCCC | 96 | 1351 |
| | | | | | TGGCTG | | |
| 833468 | N/A | N/A | 4872 | 4887 | ATCCGACCCC | 93 | 1352 |
| | | | | | CAGCAG | | |
| 833484 | N/A | N/A | 4903 | 4918 | GCCGTCATAA | 48 | 1353 |
| | | | | | TCCTGG | | |
| 833500 | N/A | N/A | 4921 | 4936 | TCTTTATCCT | 77 | 1354 |
| | | | | | GGTGGT | | |
| 833532 | N/A | N/A | 2724 | 2739 | CTTCGAGGTA | 92 | 1355 |
| | | | | | CTGCTA | | |
| 833548 | N/A | N/A | 3055 | 3070 | GCCCATGGCC | 52 | 1356 |
| | | | | | ATCATA | | |
| 833564 | N/A | N/A | 3596 | 3611 | AGAGAAGCCC | 70 | 1357 |
| | | | | | TATCGA | | |
| 833580 | N/A | N/A | 3795 | 3810 | GGGTTATACT | 39 | 1358 |
| | | | | | CAGGCT | | |
| 833596 | N/A | N/A | 4043 | 4058 | GCCCAGGAGA | 92 | 1359 |
| | | | | | CCGGCT | | |
| 833612 | N/A | N/A | 5763 | 5778 | CGCCGTACCT | 63 | 1360 |
| | | | | | CCCAGC | | |
| 833628 | N/A | N/A | 6036 | 6051 | GAAATTGCCA | 64 | 1361 |
| | | | | | TTCACG | | |
| 833644 | N/A | N/A | 6618 | 6633 | GAACGAAGAC | 65 | 1362 |
| | | | | | CTCCAC | | |
| 833660 | N/A | N/A | 6938 | 6953 | ACTTGACGGA | 79 | 1363 |
| | | | | | CAAGGG | | |
| 833676 | N/A | N/A | 7428 | 7443 | GACGAGGTGG | 100 | 1364 |
| | | | | | GTTTCT | | |
| 833692 | N/A | N/A | 7927 | 7942 | AAAAGCTGGG | 98 | 1365 |
| | | | | | CTACCC | | |
| 833708 | N/A | N/A | 8466 | 8481 | AGCAAAAGAT | 111 | 1366 |
| | | | | | GCCCTC | | |
| 833724 | N/A | N/A | 8951 | 8966 | TGCCATGTCC | 68 | 1367 |
| | | | | | AGGGTC | | |
| 833740 | N/A | N/A | 9375 | 9390 | TTATTAGCAG | 66 | 1368 |
| | | | | | CAGGGT | | |
| 833756 | N/A | N/A | 10000 | 10015 | GGCTTACTGG | 47 | 1369 |
| | | | | | TCAGGC | | |
| 833772 | N/A | N/A | 10694 | 10709 | ATAATGACAC | 63 | 1370 |
| | | | | | ATCCTT | | |
| 833787 | N/A | N/A | 11415 | 11430 | GGATAGCATA | 55 | 1371 |
| | | | | | GCTTGA | | |
| 833803 | N/A | N/A | 11770 | 11785 | CCGCAGTCTG | 84 | 1372 |
| | | | | | GTTTAA | | |
| 833819 | N/A | N/A | 12413 | 12428 | ACATTCTGGG | 74 | 1373 |
| | | | | | ATGGCA | | |
| 833835 | N/A | N/A | 12710 | 12725 | ACCCGGAGGC | 97 | 1374 |
| | | | | | AGTGCC | | |
| 833851 | N/A | N/A | 13063 | 13078 | TTTGACAAGG | 77 | 1375 |
| | | | | | ATAGGA | | |
| 833867 | N/A | N/A | 13358 | 13373 | CGACATGGTT | 102 | 1376 |
| | | | | | GGGCAG | | |
| 833883 | N/A | N/A | 14160 | 14175 | CACTAGAGGT | 101 | 1377 |
| | | | | | GGACAG | | |
| 833899 | N/A | N/A | 14592 | 14607 | TCAACAGATA | 65 | 1378 |
| | | | | | TGGTGC | | |
| 833915 | N/A | N/A | 15478 | 15493 | CACTATCATG | 59 | 1379 |
| | | | | | TCAGCC | | |
| 833931 | N/A | N/A | 16008 | 16023 | CCCCACTTAA | 125 | 1380 |
| | | | | | TAAGCC | | |
| 833947 | N/A | N/A | 16508 | 16523 | AAAGGACCTT | 110 | 1381 |
| | | | | | TCGAGG | | |
| 833963 | N/A | N/A | 17424 | 17439 | GTTCATGGTA | 69 | 1382 |
| | | | | | CACTAC | | |
| 833979 | N/A | N/A | 18018 | 18033 | GACAATTTAG | 60 | 1383 |
| | | | | | ACACTC | | |
| 833995* | N/A | N/A | 18920 | 18935 | GGACACAGAT | 99 | 1384 |
| | | | | | TGCACA | | |
| 834011* | N/A | N/A | 19278 | 19293 | TCCCGGTACA | 92 | 1385 |
| | | | | | GGTGAG | | |
| 834027* | N/A | N/A | 19527 | 19542 | CAGGAGGGCC | 124 | 1386 |
| | | | | | CCGAGA | | |

TABLE 19
Reduction of SPDEF RNA by 4 μM 3-10-3 cEt
gapmers with a phosphorothioate backbone

| | SEQ | SEQ | SEQ | SEQ | | | |
|---|---|---|---|---|---|---|---|
| | ID | ID | ID | ID | | | |
| | NO: | NO: | NO: | NO: | | | |
| Com- | 1 | 1 | 2 | 2 | | SPDEF | SEQ |
| pound | Start | Stop | Start | Stop | Sequence | (% | ID |
| Number | Site | Site | Site | Site | (5′ to 3′) | UTC) | NO |
| 801766 | 712 | 727 | 13823 | 13838 | CTGTCAATGA | 60 | 33 |
| | | | | | CCGGGC | | |
| 832835 | 51 | 66 | 1718 | 1733 | AGTGTGGACA | 71 | 1387 |
| | | | | | CGGCAG | | |
| 832851 | 131 | 146 | 1798 | 1813 | CTGCTGGCAC | 73 | 1388 |
| | | | | | CGTGGC | | |
| 832867 | 238 | 253 | 1905 | 1920 | AGCACTCAGG | 100 | 1389 |
| | | | | | TTGGCC | | |
| 832883 | 404 | 419 | 13515 | 13530 | GCTGGCGGCT | 96 | 1390 |
| | | | | | GTGTCT | | |
| 832899 | 444 | 459 | 13555 | 13570 | TCAGACCCGG | 87 | 1391 |
| | | | | | GCTGGC | | |
| 832915 | 504 | 519 | 13615 | 13630 | CCAAGCCTGT | 58 | 1392 |
| | | | | | CCGCGA | | |
| 832931 | 553 | 568 | 13664 | 13679 | GGACTCCAGT | 99 | 1393 |
| | | | | | CCCGTC | | |
| 832947 | 597 | 612 | 13708 | 13723 | GGTAGAAGGC | 76 | 1394 |
| | | | | | GGACAG | | |
| 832963 | 673 | 688 | 13784 | 13799 | TCCTCCCGAC | 79 | 1395 |
| | | | | | TGCTGG | | |
| 832979 | 752 | 767 | 13863 | 13878 | CCCGCCGGGC | 75 | 1396 |
| | | | | | ACCAAG | | |
| 832995 | 794 | 809 | 13905 | 13920 | CATGGACTGC | 77 | 1397 |
| | | | | | ACCTGC | | |
| 833011 | 826 | 841 | 13937 | 13952 | GTCTCGATGT | 79 | 1398 |
| | | | | | CCTTGA | | |
| 833026 | 858 | 873 | N/A | N/A | GATCTGCGGT | 93 | 1399 |
| | | | | | GATGTT | | |
| 833042 | 964 | 979 | 16913 | 16928 | TCCTTGCCCG | 80 | 1400 |
| | | | | | CCAGCT | | |
| 833056 | 994 | 1009 | 16943 | 16958 | CGGAACTGCT | 87 | 1401 |
| | | | | | CCTCCG | | |
| 833068 | 1068 | 1083 | 18438 | 18453 | CTTTCATCCA | 70 | 1402 |
| | | | | | GGCCGC | | |
| 833084 | 1097 | 1112 | 18467 | 18482 | GTAGTGAATC | 64 | 1403 |
| | | | | | GCCCCA | | |
| 833100 | 1129 | 1144 | 18617 | 18632 | TCGGTCCAGC | 108 | 1404 |
| | | | | | TCTCCT | | |
| 833116 | 1155 | 1170 | 18643 | 18658 | CGGAGCATGA | 116 | 1405 |
| | | | | | TGAGTC | | |
| 833130 | 1205 | 1220 | 18693 | 18708 | GGGCTTGAGT | 92 | 1406 |
| | | | | | AGCAAC | | |
| 833144* | 1243 | 1258 | 18731 | 18746 | TCCTTGTTGA | 10 | 1407 |
| | | | | | GCCACC | | |
| 833149 | 1384 | 1399 | 19669 | 19684 | TCTGGCTTCC | 74 | 1408 |
| | | | | | GGATGA | | |
| 833165 | 1521 | 1536 | 19806 | 19821 | GACTGCCCGT | 63 | 1409 |
| | | | | | TTTCCC | | |
| 833181 | 1615 | 1630 | 19900 | 19915 | AGGGTCCCGA | 86 | 1410 |
| | | | | | AGGCCC | | |
| 833197 | 1764 | 1779 | 20049 | 20064 | GGACTGCCTG | 69 | 1411 |
| | | | | | TGGCCT | | |
| 833213 | N/A | N/A | 4951 | 4966 | GTGCCAGAGC | 71 | 1412 |
| | | | | | TAGAGG | | |
| 833229 | N/A | N/A | 5020 | 5035 | CCTTGCCCCG | 93 | 1413 |
| | | | | | CAACCA | | |
| 833245 | N/A | N/A | 5057 | 5072 | AAAGCAACTT | 80 | 1414 |
| | | | | | GCTACC | | |
| 833261 | N/A | N/A | 5158 | 5173 | GACCGGTTCC | 89 | 1415 |
| | | | | | CACCTG | | |
| 833277 | N/A | N/A | 5210 | 5225 | TGCTTCAAAC | 49 | 1416 |
| | | | | | AAGGTT | | |
| 833293 | N/A | N/A | 5282 | 5297 | AAGTGAAGGA | 69 | 1417 |
| | | | | | CTCCAA | | |
| 833309 | N/A | N/A | 5347 | 5362 | ATTCCTGCGC | 57 | 1418 |
| | | | | | CCTGAT | | |
| 833325 | N/A | N/A | 5390 | 5405 | GGTTGTTGGT | 84 | 1419 |
| | | | | | CTGTAA | | |
| 833341 | N/A | N/A | 5456 | 5471 | GCCGTGTTTC | 77 | 1420 |
| | | | | | TGCGGC | | |
| 833357 | N/A | N/A | 5498 | 5513 | CCGAGAGGTG | 97 | 1421 |
| | | | | | TGTGTC | | |
| 833373 | N/A | N/A | 5525 | 5540 | AGCGATGTGA | 99 | 1422 |
| | | | | | GAAGGC | | |
| 833389 | N/A | N/A | 5581 | 5596 | AACAGGGACT | 123 | 1423 |
| | | | | | CATAAA | | |
| 833405 | N/A | N/A | 5627 | 5642 | GAGGCCCCTT | 95 | 1424 |
| | | | | | GGCCGA | | |
| 833421 | N/A | N/A | 5661 | 5676 | CACGGATGTC | 80 | 1425 |
| | | | | | ACCGGA | | |
| 833437 | N/A | N/A | 5728 | 5743 | GTGAGGTTTC | 85 | 1426 |
| | | | | | CTGGGT | | |
| 833453 | N/A | N/A | 4850 | 4865 | ACTAAGGTCC | 85 | 1427 |
| | | | | | CTGGCT | | |
| 833469 | N/A | N/A | 4873 | 4888 | CATCCGACCC | 112 | 1428 |
| | | | | | CCAGCA | | |
| 833485 | N/A | N/A | 4904 | 4919 | CGCCGTCATA | 74 | 1429 |
| | | | | | ATCCTG | | |
| 833501 | N/A | N/A | 18265 | 18280 | TTACAAGAAG | 112 | 1430 |
| | | | | | CTGCTT | | |
| 833533 | N/A | N/A | 2735 | 2750 | GAAATGAGCA | 76 | 1431 |
| | | | | | CCTTCG | | |
| 833549 | N/A | N/A | 3126 | 3141 | ATGCAGCTTT | 65 | 1432 |
| | | | | | ATTGGG | | |
| 833565 | N/A | N/A | 3611 | 3626 | TCAGACTTGG | 62 | 1433 |
| | | | | | TTGACA | | |
| 833581 | N/A | N/A | 3799 | 3814 | CCCCGGGTTA | 46 | 1434 |
| | | | | | TACTCA | | |
| 833597 | N/A | N/A | 4268 | 4283 | AAGAAGCGGA | 79 | 1435 |
| | | | | | AGGTGA | | |
| 833613 | N/A | N/A | 5768 | 5783 | GCATACGCCG | 76 | 1436 |
| | | | | | TACCTC | | |
| 833629 | N/A | N/A | 6067 | 6082 | TACAATTCCG | 83 | 1437 |
| | | | | | CTCAAC | | |
| 833645 | N/A | N/A | 6620 | 6635 | AAGAACGAAG | 66 | 1438 |
| | | | | | ACCTCC | | |
| 833661 | N/A | N/A | 6940 | 6955 | CCACTTGACG | 89 | 1439 |
| | | | | | GACAAG | | |
| 833677 | N/A | N/A | 7437 | 7452 | GCATGAGTAG | 81 | 1440 |
| | | | | | ACGAGG | | |
| 833693 | N/A | N/A | 8036 | 8051 | CCTTAAATGG | 97 | 1441 |
| | | | | | GCTGGA | | |
| 833709 | N/A | N/A | 8480 | 8495 | CCCCAACTGG | 91 | 1442 |
| | | | | | CATCAG | | |
| 833725 | N/A | N/A | 8986 | 9001 | CCGTAGGCCA | 96 | 1443 |
| | | | | | AGGGTC | | |
| 833741 | N/A | N/A | 9377 | 9392 | GCTTATTAGC | 52 | 1444 |
| | | | | | AGCAGG | | |
| 833757 | N/A | N/A | 10083 | 10098 | GGAAAGGTTC | 64 | 1445 |
| | | | | | GACTCT | | |
| 833773 | N/A | N/A | 10699 | 10714 | GGCATATAAT | 46 | 1446 |
| | | | | | GACACA | | |
| 833788 | N/A | N/A | 11417 | 11432 | CTGGATAGCA | 79 | 1447 |
| | | | | | TAGCTT | | |
| 833804 | N/A | N/A | 11847 | 11862 | CGCCACCTCG | 97 | 1448 |
| | | | | | GAGCTT | | |
| 833820 | N/A | N/A | 12431 | 12446 | AAGCACTGAA | 95 | 1449 |
| | | | | | ACCCCA | | |
| 833836 | N/A | N/A | 12722 | 12737 | GAGCATGCGG | 92 | 1450 |
| | | | | | CCACCC | | |
| 833852 | N/A | N/A | 13066 | 13081 | GCCTTTGACA | 75 | 1451 |
| | | | | | AGGATA | | |
| 833868 | N/A | N/A | 13360 | 13375 | CACGACATGG | 46 | 1452 |
| | | | | | TTGGGC | | |
| 833884 | N/A | N/A | 14162 | 14177 | GACACTAGAG | 95 | 1453 |
| | | | | | GTGGAC | | |
| 833900 | N/A | N/A | 14594 | 14609 | GATCAACAGA | 82 | 1454 |
| | | | | | TATGGT | | |
| 833916 | N/A | N/A | 15561 | 15576 | CTAGGAGGTC | 81 | 1455 |
| | | | | | CCCTCC | | |
| 833932 | N/A | N/A | 16063 | 16078 | GTGAACACCA | 50 | 1456 |
| | | | | | TGGTCC | | |
| 833948 | N/A | N/A | 16512 | 16527 | CTCCAAAGGA | 90 | 1457 |
| | | | | | CCTTTC | | |
| 833964 | N/A | N/A | 17522 | 17537 | ATAGACTTTC | 66 | 1458 |
| | | | | | CCTGGA | | |
| 833980 | N/A | N/A | 18118 | 18133 | TCCTATGAGT | 53 | 1459 |
| | | | | | TGGTCC | | |
| 833996* | N/A | N/A | 18956 | 18971 | TCCTAAGTGA | 66 | 1460 |
| | | | | | GACAGA | | |
| 834012* | N/A | N/A | 19286 | 19301 | CACAAACCTC | 107 | 1461 |
| | | | | | CCGGTA | | |
| 834028* | N/A | N/A | 19543 | 19558 | TTGAAGATGC | 95 | 1462 |
| | | | | | CTAGAG | | |

TABLE 20
Reduction of SPDEF RNA by 4 μM 3-10-3 cEt
gapmers with a phosphorothioate backbone

| | SEQ | SEQ | SEQ | SEQ | | | |
|---|---|---|---|---|---|---|---|
| | ID | ID | ID | ID | | | |
| | NO: | NO: | NO: | NO: | | | |
| Com- | 1 | 1 | 2 | 2 | | SPDEF | SEQ |
| pound | Start | Stop | Start | Stop | Sequence | (% | ID |
| Number | Site | Site | Site | Site | (5′ to 3′) | UTC) | NO |
| 652502* | 1219 | 1234 | 18707 | 18722 | CGGCCATAGC | 106 | 1463 |
| | | | | | TGTGGG | | |
| 801766 | 712 | 727 | 13823 | 13838 | CTGTCAATGA | 55 | 33 |
| | | | | | CCGGGC | | |
| 832836 | 53 | 68 | 1720 | 1735 | GCAGTGTGGA | 70 | 1464 |
| | | | | | CACGGC | | |
| 832852 | 160 | 175 | 1827 | 1842 | GGAGTCCCCT | 79 | 1465 |
| | | | | | ACCCCC | | |
| 832868 | 239 | 254 | 1906 | 1921 | CAGCACTCAG | 81 | 1466 |
| | | | | | GTTGGC | | |
| 832884 | 405 | 420 | 13516 | 13531 | GGCTGGCGGC | 80 | 1467 |
| | | | | | TGTGTC | | |
| 832900 | 445 | 460 | 13556 | 13571 | CTCAGACCCG | 65 | 1468 |
| | | | | | GGCTGG | | |
| 832916 | 505 | 520 | 13616 | 13631 | TCCAAGCCTG | 79 | 1469 |
| | | | | | TCCGCG | | |
| 832932 | 554 | 569 | 13665 | 13680 | GGGACTCCAG | 107 | 1470 |
| | | | | | TCCCGT | | |
| 832948 | 598 | 613 | 13709 | 13724 | AGGTAGAAGG | 87 | 1471 |
| | | | | | CGGACA | | |
| 832964 | 674 | 689 | 13785 | 13800 | CTCCTCCCGA | 84 | 1472 |
| | | | | | CTGCTG | | |
| 832980 | 753 | 768 | 13864 | 13879 | GCCCGCCGGG | 89 | 1473 |
| | | | | | CACCAA | | |
| 832996 | 795 | 810 | 13906 | 13921 | CCATGGACTG | 66 | 1474 |
| | | | | | CACCTG | | |
| 833012 | 827 | 842 | 13938 | 13953 | CGTCTCGATG | 64 | 1475 |
| | | | | | TCCTTG | | |
| 833027 | 859 | 874 | N/A | N/A | GGATCTGCGG | 83 | 1476 |
| | | | | | TGATGT | | |
| 833043 | 966 | 981 | 16915 | 16930 | GCTCCTTGCC | 75 | 1477 |
| | | | | | CGCCAG | | |
| 833057 | 996 | 1011 | 16945 | 16960 | GGCGGAACTG | 64 | 1478 |
| | | | | | CTCCTC | | |
| 833069 | 1074 | 1089 | 18444 | 18459 | TCCGCTCTTT | 72 | 1479 |
| | | | | | CATCCA | | |
| 833085 | 1099 | 1114 | 18469 | 18484 | CAGTAGTGAA | 63 | 1480 |
| | | | | | TCGCCC | | |
| 833101 | 1131 | 1146 | 18619 | 18634 | TGTCGGTCCA | 108 | 1481 |
| | | | | | GCTCTC | | |
| 833117 | 1157 | 1172 | 18645 | 18660 | CCCGGAGCAT | 38 | 1482 |
| | | | | | GATGAG | | |
| 833145* | 1244 | 1259 | 18732 | 18747 | CTCCTTGTTG | 14 | 1483 |
| | | | | | AGCCAC | | |
| 833150 | 1385 | 1400 | 19670 | 19685 | GTCTGGCTTC | 101 | 1484 |
| | | | | | CGGATG | | |
| 833166 | 1522 | 1537 | 19807 | 19822 | AGACTGCCCG | 79 | 1485 |
| | | | | | TTTTCC | | |
| 833182 | 1617 | 1632 | 19902 | 19917 | CCAGGGTCCC | 86 | 1486 |
| | | | | | GAAGGC | | |
| 833198 | 1765 | 1780 | 20050 | 20065 | TGGACTGCCT | 59 | 1487 |
| | | | | | GTGGCC | | |
| 833214 | N/A | N/A | 4952 | 4967 | TGTGCCAGAG | 81 | 1488 |
| | | | | | CTAGAG | | |
| 833230 | N/A | N/A | 5021 | 5036 | GCCTTGCCCC | 78 | 1489 |
| | | | | | GCAACC | | |
| 833246 | N/A | N/A | 5064 | 5079 | AGCCCTCAAA | 81 | 1490 |
| | | | | | GCAACT | | |
| 833262 | N/A | N/A | 5159 | 5174 | GGACCGGTTC | 68 | 1491 |
| | | | | | CCACCT | | |
| 833278 | N/A | N/A | 5211 | 5226 | TTGCTTCAAA | 65 | 1492 |
| | | | | | CAAGGT | | |
| 833294 | N/A | N/A | 5283 | 5298 | AAAGTGAAGG | 83 | 1493 |
| | | | | | ACTCCA | | |
| 833310 | N/A | N/A | 5348 | 5363 | CATTCCTGCG | 72 | 1494 |
| | | | | | CCCTGA | | |
| 833326 | N/A | N/A | 5391 | 5406 | TGGTTGTTGG | 98 | 1495 |
| | | | | | TCTGTA | | |
| 833342 | N/A | N/A | 5457 | 5472 | TGCCGTGTTT | 82 | 1496 |
| | | | | | CTGCGG | | |
| 833358 | N/A | N/A | 5499 | 5514 | CCCGAGAGGT | 102 | 1497 |
| | | | | | GTGTGT | | |
| 833374 | N/A | N/A | 5526 | 5541 | CAGCGATGTG | 104 | 1498 |
| | | | | | AGAAGG | | |
| 833390 | N/A | N/A | 5582 | 5597 | AAACAGGGAC | 94 | 1499 |
| | | | | | TCATAA | | |
| 833406 | N/A | N/A | 5628 | 5643 | TGAGGCCCCT | 82 | 1500 |
| | | | | | TGGCCG | | |
| 833422 | N/A | N/A | 5663 | 5678 | CACACGGAT | 85 | 1501 |
| | | | | | GTCACCG | | |
| 833438 | N/A | N/A | 5749 | 5764 | GCTTGCCACA | 63 | 1502 |
| | | | | | GGACAG | | |
| 833454 | N/A | N/A | 4851 | 4866 | CACTAAGGTC | 84 | 1503 |
| | | | | | CCTGGC | | |
| 833470 | N/A | N/A | 4874 | 4889 | CCATCCGACC | 86 | 1504 |
| | | | | | CCCAGC | | |
| 833486 | N/A | N/A | 4905 | 4920 | GCGCCGTCAT | 58 | 1505 |
| | | | | | AATCCT | | |
| 833502 | N/A | N/A | 18269 | 18284 | CTGGTTACAA | 65 | 1506 |
| | | | | | GAAGCT | | |
| 833518 | N/A | N/A | 2090 | 2105 | TGCCAGGGTA | 75 | 1507 |
| | | | | | CCCCCA | | |
| 833534 | N/A | N/A | 2737 | 2752 | TAGAAATGAG | 95 | 1508 |
| | | | | | CACCTT | | |
| 833550 | N/A | N/A | 3256 | 3271 | GCTGAACCAT | 89 | 1509 |
| | | | | | GGCCTG | | |
| 833566 | N/A | N/A | 3613 | 3628 | CCTCAGACTT | 84 | 1510 |
| | | | | | GGTTGA | | |
| 833582 | N/A | N/A | 3807 | 3822 | ATTAACTTCC | 80 | 1511 |
| | | | | | CCGGGT | | |
| 833598 | N/A | N/A | 4367 | 4382 | GTTGAGTGTA | 87 | 1512 |
| | | | | | CATGAG | | |
| 833614 | N/A | N/A | 5771 | 5786 | CCCGCATACG | 77 | 1513 |
| | | | | | CCGTAC | | |
| 833630 | N/A | N/A | 6069 | 6084 | ACTACAATTC | 70 | 1514 |
| | | | | | CGCTCA | | |
| 833646 | N/A | N/A | 6623 | 6638 | GGAAAGAAC | 86 | 1515 |
| | | | | | GAAGACC | | |
| 833662 | N/A | N/A | 6942 | 6957 | TCCCACTTG | 75 | 1516 |
| | | | | | ACGGACA | | |
| 833678 | N/A | N/A | 7439 | 7454 | CAGCATGAGT | 54 | 1517 |
| | | | | | AGACGA | | |
| 833694 | N/A | N/A | 8091 | 8106 | GGCCTACTGA | 83 | 1518 |
| | | | | | GCTGTC | | |
| 833710 | N/A | N/A | 8530 | 8545 | CTAGAAATGT | 92 | 1519 |
| | | | | | GCCCCT | | |
| 833726 | N/A | N/A | 8989 | 9004 | GCTCCGTAGG | 63 | 1520 |
| | | | | | CCAAGG | | |
| 833742 | N/A | N/A | 9391 | 9406 | GAAGGGATTC | 78 | 1521 |
| | | | | | ATGTGC | | |
| 833758 | N/A | N/A | 10086 | 10101 | GGAGGAAAGG | 60 | 1522 |
| | | | | | TTCGAC | | |
| 833774 | N/A | N/A | 10714 | 10729 | AGCTTTTGCC | 78 | 1523 |
| | | | | | AGGAAG | | |
| 833789 | N/A | N/A | 11419 | 11434 | CCCTGGATAG | 62 | 1524 |
| | | | | | CATAGC | | |
| 833805 | N/A | N/A | 11880 | 11895 | CTCCAAATGT | 63 | 1525 |
| | | | | | GCCGTC | | |
| 833821 | N/A | N/A | 12434 | 12449 | CGCAAGCACT | 81 | 1526 |
| | | | | | GAAACC | | |
| 833837 | N/A | N/A | 12737 | 12752 | CCCCGATGCC | 102 | 1527 |
| | | | | | TGGAGG | | |
| 833853 | N/A | N/A | 13092 | 13107 | GATATAGCAA | 62 | 1528 |
| | | | | | AGCTTG | | |
| 833869 | N/A | N/A | 13363 | 13378 | AGCCACGACA | 102 | 1529 |
| | | | | | TGGTTG | | |
| 833885 | N/A | N/A | 14208 | 14223 | TATCATCCAG | 71 | 1530 |
| | | | | | CACCTA | | |
| 833901 | N/A | N/A | 14599 | 14614 | AGCGAGATCA | 55 | 1531 |
| | | | | | ACAGAT | | |
| 833917 | N/A | N/A | 15563 | 15578 | CCCTAGGAGG | 81 | 1532 |
| | | | | | TCCCCT | | |
| 833933 | N/A | N/A | 16089 | 16104 | GGGCATGGTC | 72 | 1533 |
| | | | | | ACAATG | | |
| 833949 | N/A | N/A | 16570 | 16585 | GTGCATCTGT | 67 | 1534 |
| | | | | | ACTGCC | | |
| 833965 | N/A | N/A | 17533 | 17548 | CTCGAGTATT | 58 | 1535 |
| | | | | | CATAGA | | |
| 833981 | N/A | N/A | 18128 | 18143 | TCTGACAGGG | 68 | 1536 |
| | | | | | TCCTAT | | |
| 833997* | N/A | N/A | 18968 | 18983 | CAGTACTAAA | 68 | 1537 |
| | | | | | ACTCCT | | |
| 834013* | N/A | N/A | 19300 | 19315 | GAATACTCTG | 96 | 1538 |
| | | | | | GAGTCA | | |
| 834029 | N/A | N/A | 20191 | 20206 | GGACATGTCA | 89 | 1539 |
| | | | | | GTTCTC | | |

TABLE 21
Reduction of SPDEF RNA by 4 μM 3-10-3 cEt
gapmers with a phosphorothioate backbone

| | SEQ | SEQ | SEQ | SEQ | | | |
|---|---|---|---|---|---|---|---|
| | ID | ID | ID | ID | | | |
| | NO: | NO: | NO: | NO: | | | |
| Com- | 1 | 1 | 2 | 2 | | SPDEF | SEQ |
| pound | Start | Stop | Start | Stop | Sequence | (% | ID |
| Number | Site | Site | Site | Site | (5′ to 3′) | UTC) | NO |
| 652506* | 1245 | 1260 | 18733 | 18748 | TCTCCTTGTT | 22 | 1540 |
| | | | | | GAGCCA | | |
| 801766 | 712 | 727 | 13823 | 13838 | CTGTCAATGA | 71 | 33 |
| | | | | | CCGGGC | | |
| 832837 | 71 | 86 | 1738 | 1753 | GAGGACTGGG | 91 | 1541 |
| | | | | | TCTGTG | | |
| 832853 | 161 | 176 | 1828 | 1843 | GGGAGTCCCC | 106 | 1542 |
| | | | | | TACCCC | | |
| 832869 | 240 | 255 | 1907 | 1922 | GCAGCACTCA | 106 | 1543 |
| | | | | | GGTTGG | | |
| 832885 | 407 | 422 | 13518 | 13533 | TGGGCTGGCG | 78 | 1544 |
| | | | | | GCTGTG | | |
| 832901 | 446 | 461 | 13557 | 13572 | GCTCAGACCC | 90 | 1545 |
| | | | | | GGGCTG | | |
| 832917 | 507 | 522 | 13618 | 13633 | TCTCCAAGCC | 75 | 1546 |
| | | | | | TGTCCG | | |
| 832933 | 558 | 573 | 13669 | 13684 | GACTGGGACT | 86 | 1547 |
| | | | | | CCAGTC | | |
| 832949 | 601 | 616 | 13712 | 13727 | GAGAGGTAGA | 78 | 1548 |
| | | | | | AGGCGG | | |
| 832965 | 675 | 690 | 13786 | 13801 | GCTCCTCCCG | 63 | 1549 |
| | | | | | ACTGCT | | |
| 832981 | 754 | 769 | 13865 | 13880 | AGCCCGCCGG | 94 | 1550 |
| | | | | | GCACCA | | |
| 832997 | 798 | 813 | 13909 | 13924 | CCACCATGGA | 73 | 1551 |
| | | | | | CTGCAC | | |
| 833013 | 829 | 844 | 13940 | 13955 | GCCGTCTCGA | 47 | 1552 |
| | | | | | TGTCCT | | |
| 833028 | 862 | 877 | N/A | N/A | ATGGGATCTG | 84 | 1553 |
| | | | | | CGGTGA | | |
| 833044 | 968 | 983 | 16917 | 16932 | CAGCTCCTTG | 80 | 1554 |
| | | | | | CCCGCC | | |
| 833058 | 997 | 1012 | 16946 | 16961 | TGGCGGAACT | 102 | 1555 |
| | | | | | GCTCCT | | |
| 833070 | 1076 | 1091 | 18446 | 18461 | AGTCCGCTCT | 83 | 1556 |
| | | | | | TTCATC | | |
| 833086 | 1101 | 1116 | 18471 | 18486 | CACAGTAGTG | 85 | 1557 |
| | | | | | AATCGC | | |
| 833102 | 1132 | 1147 | 18620 | 18635 | CTGTCGGTCC | 82 | 1558 |
| | | | | | AGCTCT | | |
| 833118 | 1159 | 1174 | 18647 | 18662 | TGCCCGGAGC | 103 | 1559 |
| | | | | | ATGATG | | |
| 833131* | 1221 | 1236 | 18709 | 18724 | AGCGGCCATA | 90 | 1560 |
| | | | | | GCTGTG | | |
| 833151 | 1387 | 1402 | 19672 | 19687 | ATGTCTGGCT | 82 | 1561 |
| | | | | | TCCGGA | | |
| 833167 | 1523 | 1538 | 19808 | 19823 | CAGACTGCCC | 72 | 1562 |
| | | | | | GTTTTC | | |
| 833183 | 1618 | 1633 | 19903 | 19918 | CCCAGGGTCC | 106 | 1563 |
| | | | | | CGAAGG | | |
| 833199 | 1834 | 1849 | 20119 | 20134 | AGATGTCTCC | 67 | 1564 |
| | | | | | CTGCAC | | |
| 833215 | N/A | N/A | 4953 | 4968 | CTGTGCCAGA | 90 | 1565 |
| | | | | | GCTAGA | | |
| 833231 | N/A | N/A | 5022 | 5037 | GGCCTTGCCC | 99 | 1566 |
| | | | | | CGCAAC | | |
| 833247 | N/A | N/A | 5077 | 5092 | GCTAGGTCCC | 91 | 1567 |
| | | | | | AGCAGC | | |
| 833263 | N/A | N/A | 5160 | 5175 | AGGACCGGTT | 61 | 1568 |
| | | | | | CCCACC | | |
| 833279 | N/A | N/A | 5230 | 5245 | GACAGGCTAA | 87 | 1569 |
| | | | | | GAACAG | | |
| 833295 | N/A | N/A | 5284 | 5299 | CAAAGTGAAG | 68 | 1570 |
| | | | | | GACTCC | | |
| 833311 | N/A | N/A | 5349 | 5364 | ACATTCCTGC | 64 | 1571 |
| | | | | | GCCCTG | | |
| 833327 | N/A | N/A | 5392 | 5407 | CTGGTTGTTG | 89 | 1572 |
| | | | | | GTCTGT | | |
| 833343 | N/A | N/A | 5458 | 5473 | CTGCCGTGTT | 56 | 1573 |
| | | | | | TCTGCG | | |
| 833359 | N/A | N/A | 5500 | 5515 | TCCCGAGAGG | 74 | 1574 |
| | | | | | TGTGTG | | |
| 833375 | N/A | N/A | 5527 | 5542 | ACAGCGATGT | 88 | 1575 |
| | | | | | GAGAAG | | |
| 833391 | N/A | N/A | 5586 | 5601 | AGTGAAACAG | 84 | 1576 |
| | | | | | GGACTC | | |
| 833407 | N/A | N/A | 5629 | 5644 | CTGAGGCCCC | 79 | 1577 |
| | | | | | TTGGCC | | |
| 833423 | N/A | N/A | 5664 | 5679 | ACACACGGAT | 82 | 1578 |
| | | | | | GTCACC | | |
| 833455 | N/A | N/A | 4852 | 4867 | ACACTAAGGT | 75 | 1579 |
| | | | | | CCCTGG | | |
| 833471 | N/A | N/A | 4876 | 4891 | TCCCATCCGA | 95 | 1580 |
| | | | | | CCCCCA | | |
| 833487 | N/A | N/A | 4906 | 4921 | TGCGCCGTCA | 52 | 1581 |
| | | | | | TAATCC | | |
| 833503 | N/A | N/A | 18270 | 18285 | GCTGGTTACA | 108 | 1582 |
| | | | | | AGAAGC | | |
| 833519 | N/A | N/A | 2102 | 2117 | GGAAAGACCC | 117 | 1583 |
| | | | | | CATGCC | | |
| 833535 | N/A | N/A | 2760 | 2775 | CACCGCAGAA | 69 | 1584 |
| | | | | | ATCTGG | | |
| 833551 | N/A | N/A | 3373 | 3388 | CGAGAATGCC | 71 | 1585 |
| | | | | | CCCCAC | | |
| 833567 | N/A | N/A | 3647 | 3662 | ATCGACTGAG | 42 | 1586 |
| | | | | | CACCTA | | |
| 833583 | N/A | N/A | 3878 | 3893 | CCACATGGCG | 96 | 1587 |
| | | | | | GGACCT | | |
| 833599 | N/A | N/A | 4375 | 4390 | TATGATGGGT | 102 | 1588 |
| | | | | | TGAGTG | | |
| 833615 | N/A | N/A | 5819 | 5834 | TTGAAGGGCC | 87 | 1589 |
| | | | | | GGCCAC | | |
| 833631 | N/A | N/A | 6072 | 6087 | GCAACTACAA | 54 | 1590 |
| | | | | | TTCCGC | | |
| 833647 | N/A | N/A | 6673 | 6688 | CCCCAAGTGG | 68 | 1591 |
| | | | | | ACCATC | | |
| 833663 | N/A | N/A | 6958 | 6973 | GGGCAGCCAG | 97 | 1592 |
| | | | | | CATTAT | | |
| 833679 | N/A | N/A | 7542 | 7557 | CCCATTGTGG | 82 | 1593 |
| | | | | | CCATCT | | |
| 833695 | N/A | N/A | 8099 | 8114 | TCCCATGTGG | 88 | 1594 |
| | | | | | CCTACT | | |
| 833711 | N/A | N/A | 8583 | 8598 | AGATTTAGTG | 56 | 1595 |
| | | | | | CAGCTT | | |
| 833727 | N/A | N/A | 9117 | 9132 | AGTGATGGTC | 67 | 1596 |
| | | | | | CACCCA | | |
| 833743 | N/A | N/A | 9543 | 9558 | CAAGAATCTC | 95 | 1597 |
| | | | | | CCATGG | | |
| 833759 | N/A | N/A | 10102 | 10117 | GGTTAACTGT | 76 | 1598 |
| | | | | | GTGGTT | | |
| 833775 | N/A | N/A | 10842 | 10857 | GCAGAACTCG | 84 | 1599 |
| | | | | | CTTCCC | | |
| 833790 | N/A | N/A | 11466 | 11481 | AGCTAGCCCA | 84 | 1600 |
| | | | | | TTCAAT | | |
| 833806 | N/A | N/A | 11901 | 11916 | TTATAGTTTC | 86 | 1601 |
| | | | | | AAGCAG | | |
| 833822 | N/A | N/A | 12442 | 12457 | GAGAGGTGCG | 70 | 1602 |
| | | | | | CAAGCA | | |
| 833838 | N/A | N/A | 12820 | 12835 | GTGCATGGTA | 85 | 1603 |
| | | | | | CCCACC | | |
| 833854 | N/A | N/A | 13095 | 13110 | CCTGATATAG | 66 | 1604 |
| | | | | | CAAAGC | | |
| 833870 | N/A | N/A | 13366 | 13381 | GGCAGCCACG | 78 | 1605 |
| | | | | | ACATGG | | |
| 833886 | N/A | N/A | 14213 | 14228 | ATTCATATCA | 55 | 1606 |
| | | | | | TCCAGC | | |
| 833902 | N/A | N/A | 14623 | 14638 | TTCTAGTGGA | 62 | 1607 |
| | | | | | GGACAC | | |
| 833918 | N/A | N/A | 15611 | 15626 | CCATAATCAC | 72 | 1608 |
| | | | | | GCCTTC | | |
| 833934 | N/A | N/A | 16120 | 16135 | TCATAGGCCT | 100 | 1609 |
| | | | | | ATAGGT | | |
| 833950 | N/A | N/A | 16580 | 16595 | GGGTAACCTG | 80 | 1610 |
| | | | | | GTGCAT | | |
| 833966 | N/A | N/A | 17535 | 17550 | TGCTCGAGTA | 60 | 1611 |
| | | | | | TTCATA | | |
| 833982 | N/A | N/A | 18196 | 18211 | TGCAACCCCT | 76 | 1612 |
| | | | | | TGTTCA | | |
| 833998* | N/A | N/A | 18971 | 18986 | GTGCAGTACT | 108 | 1613 |
| | | | | | AAAACT | | |
| 834014* | N/A | N/A | 19302 | 19317 | GAGAATACTC | 83 | 1614 |
| | | | | | TGGAGT | | |
| 834030 | N/A | N/A | 20211 | 20226 | ATTCACTGCG | 82 | 1615 |
| | | | | | CAGACA | | |

TABLE 22
Reduction of SPDEF RNA by 4 μM 3-10-3 cEt
gapmers with a phosphorothioate backbone

| | SEQ | SEQ | SEQ | SEQ | | | |
|---|---|---|---|---|---|---|---|
| | ID | ID | ID | ID | | | |
| | NO: | NO: | NO: | NO: | | | |
| Com- | 1 | 1 | 2 | 2 | | SPDEF | SEQ |
| pound | Start | Stop | Start | Stop | Sequence | (% | ID |
| Number | Site | Site | Site | Site | (5′ to 3′) | UTC) | NO |
| 652503* | 1222 | 1237 | 18710 | 18725 | AAGCGGCCAT | 95 | 1616 |
| | | | | | AGCTGT | | |
| 652647 | N/A | N/A | 10906 | 10921 | CGTTAGGACA | 85 | 1617 |
| | | | | | GTCTCT | | |
| 791884* | 1246 | 1261 | 18734 | 18749 | TTCTCCTTGT | 26 | 1618 |
| | | | | | TGAGCC | | |
| 801766 | 712 | 727 | 13823 | 13838 | CTGTCAATGA | 82 | 33 |
| | | | | | CCGGGC | | |
| 832838 | 73 | 88 | 1740 | 1755 | TGGAGGACTG | 91 | 1619 |
| | | | | | GGTCTG | | |
| 832854 | 162 | 177 | 1829 | 1844 | AGGGAGTCCC | 92 | 1620 |
| | | | | | CTACCC | | |
| 832870 | 354 | 369 | 2021 | 2036 | CAGTGCCAA | 75 | 1621 |
| | | | | | CTTCAGG | | |
| 832886 | 408 | 423 | 13519 | 13534 | TTGGGCTGGC | 115 | 1622 |
| | | | | | GGCTGT | | |
| 832902 | 447 | 462 | 13558 | 13573 | TGCTCAGACC | 84 | 1623 |
| | | | | | CGGGCT | | |
| 832918 | 522 | 537 | 13633 | 13648 | CCCCCGCTGC | 78 | 1624 |
| | | | | | CGCCTT | | |
| 832934 | 559 | 574 | 13670 | 13685 | GGACTGGGAC | 90 | 1625 |
| | | | | | TCCAGT | | |
| 832950 | 612 | 627 | 13723 | 13738 | TGTCAAAGTA | 100 | 1626 |
| | | | | | GGAGAG | | |
| 832966 | 677 | 692 | 13788 | 13803 | TGGCTCCTCC | 70 | 1627 |
| | | | | | CGACTG | | |
| 832982 | 756 | 771 | 13867 | 13882 | TCAGCCCGCC | 106 | 1628 |
| | | | | | GGGCAC | | |
| 832998 | 808 | 823 | 13919 | 13934 | ACTTCGCCCA | 95 | 1629 |
| | | | | | CCACCA | | |
| 833014 | 831 | 846 | 13942 | 13957 | AGGCCGTCTC | 51 | 1630 |
| | | | | | GATGTC | | |
| 833029 | 864 | 879 | N/A | N/A | CCATGGGATC | 109 | 1631 |
| | | | | | TGCGGT | | |
| 833045 | 974 | 989 | 16923 | 16938 | GGCGCACAGC | 83 | 1632 |
| | | | | | TCCTTG | | |
| 833059 | 1000 | 1015 | 16949 | 16964 | CGCTGGCGGA | 105 | 1633 |
| | | | | | ACTGCT | | |
| 833071 | 1077 | 1092 | 18447 | 18462 | AAGTCCGCTC | 118 | 1634 |
| | | | | | TTTCAT | | |
| 833087 | 1102 | 1117 | N/A | N/A | GCACAGTAGT | 90 | 1635 |
| | | | | | GAATCG | | |
| 833103 | 1133 | 1148 | 18621 | 18636 | GCTGTCGGTC | 103 | 1636 |
| | | | | | CAGCTC | | |
| 833119 | 1160 | 1175 | 18648 | 18663 | CTGCCCGGAG | 98 | 1637 |
| | | | | | CATGAT | | |
| 833152 | 1388 | 1403 | 19673 | 19688 | GATGTCTGGC | 87 | 1638 |
| | | | | | TTCCGG | | |
| 833168 | 1525 | 1540 | 19810 | 19825 | AGCAGACTGC | 99 | 1639 |
| | | | | | CCGTTT | | |
| 833184 | 1619 | 1634 | 19904 | 19919 | CCCCAGGGT | 91 | 1640 |
| | | | | | CCCGAAG | | |
| 833200 | 1880 | 1895 | 20165 | 20180 | ATTATCCATT | 75 | 1641 |
| | | | | | CCCGGG | | |
| 833216 | N/A | N/A | 4961 | 4976 | CGTCTCCCCT | 80 | 1642 |
| | | | | | GTGCCA | | |
| 833232 | N/A | N/A | 5023 | 5038 | GGGCCTTGCC | 101 | 1643 |
| | | | | | CCGCAA | | |
| 833248 | N/A | N/A | 5079 | 5094 | GAGCTAGGTC | 74 | 1644 |
| | | | | | CCAGCA | | |
| 833264 | N/A | N/A | 5161 | 5176 | CAGGACCGGT | 83 | 1645 |
| | | | | | TCCCAC | | |
| 833280 | N/A | N/A | 5231 | 5246 | TGACAGGCT | 92 | 1646 |
| | | | | | AAGAACA | | |
| 833296 | N/A | N/A | 5291 | 5306 | GTTAGGACA | 80 | 1647 |
| | | | | | AAGTGAA | | |
| 833312 | N/A | N/A | 5350 | 5365 | AACATTCCT | 86 | 1648 |
| | | | | | GCGCCCT | | |
| 833328 | N/A | N/A | 5393 | 5408 | CCTGGTTGT | 73 | 1649 |
| | | | | | TGGTCTG | | |
| 833344 | N/A | N/A | 5459 | 5474 | TCTGCCGTG | 65 | 1650 |
| | | | | | TTTCTGC | | |
| 833360 | N/A | N/A | 5501 | 5516 | CTCCCGAGAG | 93 | 1651 |
| | | | | | GTGTGT | | |
| 833376 | N/A | N/A | 5528 | 5543 | GACAGCGATG | 86 | 1652 |
| | | | | | TGAGAA | | |
| 833392 | N/A | N/A | 5597 | 5612 | GCCTCTTCAG | 75 | 1653 |
| | | | | | CAGTGA | | |
| 833408 | N/A | N/A | 5630 | 5645 | CCTGAGGCCC | 89 | 1654 |
| | | | | | CTTGGC | | |
| 833424 | N/A | N/A | 5665 | 5680 | AACACACGGA | 77 | 1655 |
| | | | | | TGTCAC | | |
| 833456 | N/A | N/A | 4853 | 4868 | CACACTAAGG | 77 | 1656 |
| | | | | | TCCCTG | | |
| 833472 | N/A | N/A | 4878 | 4893 | GGTCCCATCC | 91 | 1657 |
| | | | | | GACCCC | | |
| 833488 | N/A | N/A | 4908 | 4923 | GGTGCGCCGT | 58 | 1658 |
| | | | | | CATAAT | | |
| 833504 | N/A | N/A | 18271 | 18286 | AGCTGGTTAC | 73 | 1659 |
| | | | | | AAGAAG | | |
| 833520 | N/A | N/A | 2158 | 2173 | GGCAAAGTGC | 79 | 1660 |
| | | | | | GCCCCC | | |
| 833536 | N/A | N/A | 2763 | 2778 | CTCCACCGCA | 56 | 1661 |
| | | | | | GAAATC | | |
| 833552 | N/A | N/A | 3375 | 3390 | TCCGAGAATG | 84 | 1662 |
| | | | | | CCCCCC | | |
| 833568 | N/A | N/A | 3651 | 3666 | GAGCATCGAC | 65 | 1663 |
| | | | | | TGAGCA | | |
| 833584 | N/A | N/A | 3900 | 3915 | GAAAAGTGAC | 95 | 1664 |
| | | | | | CCGCCC | | |
| 833600 | N/A | N/A | 4410 | 4425 | GTGGAGATTG | 85 | 1665 |
| | | | | | AGATGG | | |
| 833616 | N/A | N/A | 5821 | 5836 | CCTTGAAGGG | 89 | 1666 |
| | | | | | CCGGCC | | |
| 833632 | N/A | N/A | 6076 | 6091 | CATAGCAACT | 117 | 1667 |
| | | | | | ACAATT | | |
| 833648 | N/A | N/A | 6692 | 6707 | GTACAGAGGC | 91 | 1668 |
| | | | | | CCACCG | | |
| 833664 | N/A | N/A | 6982 | 6997 | TGCTTTGCCG | 79 | 1669 |
| | | | | | GGCCCT | | |
| 833680 | N/A | N/A | 7618 | 7633 | ACAGACCACC | 81 | 1670 |
| | | | | | CCGCTG | | |
| 833696 | N/A | N/A | 8220 | 8235 | CCCCATTGAG | 106 | 1671 |
| | | | | | AAGAGC | | |
| 833712 | N/A | N/A | 8587 | 8602 | GGAGAGATTT | 90 | 1672 |
| | | | | | AGTGCA | | |
| 833728 | N/A | N/A | 9146 | 9161 | ATGCAATTCA | 74 | 1673 |
| | | | | | GCCCAG | | |
| 833744 | N/A | N/A | 9573 | 9588 | CAGCACCCTT | 75 | 1674 |
| | | | | | TCATCA | | |
| 833760 | N/A | N/A | 10108 | 10123 | GTTAATGGTT | 98 | 1675 |
| | | | | | AACTGT | | |
| 833791 | N/A | N/A | 11470 | 11485 | CCACAGCTAG | 100 | 1676 |
| | | | | | CCCATT | | |
| 833807 | N/A | N/A | 11914 | 11929 | TCTCGAGGGT | 107 | 1677 |
| | | | | | TATTTA | | |
| 833823 | N/A | N/A | 12445 | 12460 | CTGGAGAGGT | 99 | 1678 |
| | | | | | GCGCAA | | |
| 833839 | N/A | N/A | 12886 | 12901 | CAACACTCTC | 113 | 1679 |
| | | | | | AAGGTG | | |
| 833855 | N/A | N/A | 13148 | 13163 | GGCGGATGAG | 71 | 1680 |
| | | | | | CAAACT | | |
| 833871 | N/A | N/A | 13445 | 13460 | CTAAGCTGGT | 79 | 1681 |
| | | | | | TATGGG | | |
| 833887 | N/A | N/A | 14215 | 14230 | GAATTCATAT | 55 | 1682 |
| | | | | | CATCCA | | |
| 833903 | N/A | N/A | 14635 | 14650 | GTGGAGTGTA | 73 | 1683 |
| | | | | | CATTCT | | |
| 833919 | N/A | N/A | 15654 | 15669 | GAGGACTAGA | 96 | 1684 |
| | | | | | GACTCA | | |
| 833935 | N/A | N/A | 16123 | 16138 | GCCTCATAGG | 80 | 1685 |
| | | | | | CCTATA | | |
| 833951 | N/A | N/A | 16598 | 16613 | TGAACTTGGT | 60 | 1686 |
| | | | | | TCAGGG | | |
| 833967 | N/A | N/A | 17542 | 17557 | GTAAATGTGC | 66 | 1687 |
| | | | | | TCGAGT | | |
| 833983 | N/A | N/A | 18201 | 18216 | TTGCATGCAA | 73 | 1688 |
| | | | | | CCCCTT | | |
| 833999* | N/A | N/A | 18998 | 19013 | GTGGATTTGG | 77 | 1689 |
| | | | | | AGCTCG | | |
| 834015* | N/A | N/A | 19306 | 19321 | GGCAGAGAAT | 82 | 1690 |
| | | | | | ACTCTG | | |
| 834031 | N/A | N/A | 20215 | 20230 | TGCCATTCAC | 108 | 1691 |
| | | | | | TGCGCA | | |

TABLE 23
Reduction of SPDEF RNA by 4 μM 3-10-3 cEt
gapmers with a phosphorothioate backbone

| | SEQ | SEQ | SEQ | SEQ | | | |
|---|---|---|---|---|---|---|---|
| | ID | ID | ID | ID | | | |
| | NO: | NO: | NO: | NO: | | | |
| Com- | 1 | 1 | 2 | 2 | | SPDEF | SEQ |
| pound | Start | Stop | Start | Stop | Sequence | (% | ID |
| Number | Site | Site | Site | Site | (5′ to 3′) | UTC) | NO |
| 801690 | 82 | 97 | 1749 | 1764 | CAGCAGGCTT | 71 | 174 |
| | | | | | GGAGGA | | |
| 802055 | N/A | N/A | 12531 | 12546 | CCCCACGGGC | 51 | 387 |
| | | | | | CGCCCC | | |
| 854164 | N/A | N/A | 2089 | 2104 | GCCAGGGTAC | 59 | 1692 |
| | | | | | CCCCAC | | |
| 854170 | N/A | N/A | 2129 | 2144 | TGCAGTCGCC | 61 | 1693 |
| | | | | | CACCCC | | |
| 854176 | N/A | N/A | 2135 | 2150 | CCTGCCTGCA | 60 | 1694 |
| | | | | | GTCGCC | | |
| 854182 | N/A | N/A | 2153 | 2168 | AGTGCGCCCC | 46 | 1695 |
| | | | | | CTCCAA | | |
| 854188 | N/A | N/A | 2160 | 2175 | CTGGCAAAGT | 78 | 1696 |
| | | | | | GCGCCC | | |
| 854194 | N/A | N/A | 2362 | 2377 | ATCTGCACGG | 67 | 1697 |
| | | | | | CGGCCT | | |
| 854200 | N/A | N/A | 3362 | 3377 | CCCACCATTT | 73 | 1698 |
| | | | | | GTCTGT | | |
| 854206 | N/A | N/A | 3380 | 3395 | GGAGCTCCGA | 68 | 1699 |
| | | | | | GAATGC | | |
| 854212 | N/A | N/A | 3680 | 3695 | TTGCACTTCC | 72 | 1700 |
| | | | | | TGCCAG | | |
| 854218 | N/A | N/A | 3689 | 3704 | TCCCGGTTTT | 85 | 1701 |
| | | | | | TGCACT | | |
| 854224 | N/A | N/A | 3695 | 3710 | GGGTTCTCCC | 58 | 1702 |
| | | | | | GGTTTT | | |
| 854230 | N/A | N/A | 3703 | 3718 | TAAAAAGTGG | 71 | 1703 |
| | | | | | GTTCTC | | |
| 854236 | N/A | N/A | 3720 | 3735 | CACAACGACC | 90 | 1704 |
| | | | | | TCAGTG | | |
| 854242 | N/A | N/A | 4486 | 4501 | CAGTGACTCA | 70 | 1705 |
| | | | | | GCCCCC | | |
| 854248 | N/A | N/A | 5764 | 5779 | ACGCCGTACC | 65 | 1706 |
| | | | | | TCCCAG | | |
| 854254 | N/A | N/A | 5772 | 5787 | CCCCGCATAC | 44 | 1707 |
| | | | | | GCCGTA | | |
| 854260 | N/A | N/A | 5808 | 5823 | GCCACAGTAC | 62 | 1708 |
| | | | | | CTTCCC | | |
| 854266 | N/A | N/A | 6298 | 6313 | GAGTTGATGT | 67 | 1709 |
| | | | | | CTGGAG | | |
| 854272 | N/A | N/A | 6304 | 6319 | TCCCTGGAGT | 77 | 1710 |
| | | | | | TGATGT | | |
| 854278 | N/A | N/A | 7384 | 7399 | TCTGGGCACA | 80 | 1711 |
| | | | | | AAACTG | | |
| 854284 | N/A | N/A | 7411 | 7426 | CTAGATCTCC | 58 | 1712 |
| | | | | | GGGCTT | | |
| 854290 | N/A | N/A | 7420 | 7435 | GGGTTTCTTC | 65 | 1713 |
| | | | | | TAGATC | | |
| 854296 | N/A | N/A | 7435 | 7450 | ATGAGTAGAC | 76 | 1714 |
| | | | | | GAGGTG | | |
| 854302 | N/A | N/A | 8811 | 8826 | TGGATTAAGG | 29 | 1715 |
| | | | | | CTCAGC | | |
| 854308 | N/A | N/A | 8820 | 8835 | CCACATGTCT | 82 | 1716 |
| | | | | | GGATTA | | |
| 854313 | N/A | N/A | 8831 | 8846 | TCTGGATTAA | 51 | 1717 |
| | | | | | GCCACA | | |
| 854319 | N/A | N/A | 8845 | 8860 | TCGAGTTGAT | 51 | 1718 |
| | | | | | CTCTTC | | |
| 854325 | N/A | N/A | 8852 | 8867 | CCAAACCTCG | 80 | 1719 |
| | | | | | AGTTGA | | |
| 854331 | N/A | N/A | 9119 | 9134 | TCAGTGATGG | 54 | 1720 |
| | | | | | TCCACC | | |
| 854337 | N/A | N/A | 9147 | 9162 | CATGCAATTC | 48 | 1721 |
| | | | | | AGCCCA | | |
| 854343 | N/A | N/A | 9847 | 9862 | CCCGCTTTCC | 50 | 1722 |
| | | | | | TACCCA | | |
| 854349 | N/A | N/A | 9853 | 9868 | ACATCACCCG | 89 | 1723 |
| | | | | | CTTTCC | | |
| 854355 | N/A | N/A | 9866 | 9881 | CAGGCTTTCA | 48 | 1724 |
| | | | | | CCCACA | | |
| 854361 | N/A | N/A | 9873 | 9888 | CCACGCCCAG | 85 | 1725 |
| | | | | | GCTTTC | | |
| 854367 | N/A | N/A | 9886 | 9901 | GTGAGCACCA | 87 | 1726 |
| | | | | | GTGCCA | | |
| 854373 | N/A | N/A | 9911 | 9926 | AGAACGGCAC | 69 | 1727 |
| | | | | | TGTGAG | | |
| 854379 | N/A | N/A | 9968 | 9983 | CCCAACCTGC | 96 | 1728 |
| | | | | | AACTAG | | |
| 854385 | N/A | N/A | 9979 | 9994 | GGGCTGGTGT | 84 | 1729 |
| | | | | | GCCCAA | | |
| 854391 | N/A | N/A | 10002 | 10017 | TTGGCTTACT | 72 | 1730 |
| | | | | | GGTCAG | | |
| 854397 | N/A | N/A | 10143 | 10158 | GGTCCTAGCT | 68 | 1731 |
| | | | | | CCAACA | | |
| 854403 | N/A | N/A | 10149 | 10164 | AGCCTCGGTC | 80 | 1732 |
| | | | | | CTAGCT | | |
| 854409 | N/A | N/A | 10165 | 10180 | AATCTACTCC | 72 | 1733 |
| | | | | | CCACCA | | |
| 854415 | N/A | N/A | 10171 | 10186 | CCAAGGAATC | 56 | 1734 |
| | | | | | TACTCC | | |
| 854421 | N/A | N/A | 10187 | 10202 | GCCCTATACC | 96 | 1735 |
| | | | | | TAAATG | | |
| 854427 | N/A | N/A | 10193 | 10208 | GACCTTGCCC | 70 | 1736 |
| | | | | | TATACC | | |
| 854433 | N/A | N/A | 11250 | 11265 | TCCCATTCAA | 71 | 1737 |
| | | | | | GGGCTC | | |
| 854439 | N/A | N/A | 11277 | 11292 | GAAGGGTGTT | 98 | 1738 |
| | | | | | CCCTTT | | |
| 854445 | N/A | N/A | 11600 | 11615 | GGCTCCCTGA | 70 | 1739 |
| | | | | | TCCATC | | |
| 854451 | N/A | N/A | 11632 | 11647 | GGTGCCCTAC | 61 | 1740 |
| | | | | | TGGGAC | | |
| 854457 | N/A | N/A | 11638 | 11653 | ATTTATGGTG | 68 | 1741 |
| | | | | | CCCTAC | | |
| 854463 | N/A | N/A | 11654 | 11669 | CCTCTCTAAC | 63 | 1742 |
| | | | | | AGTGAC | | |
| 854469 | N/A | N/A | 12002 | 12017 | GATATACGCT | 71 | 1743 |
| | | | | | CCTAAT | | |
| 854475 | N/A | N/A | 12010 | 12025 | TACCAACAGA | 87 | 1744 |
| | | | | | TATACG | | |
| 854481 | N/A | N/A | 12369 | 12384 | ACATCAAGAC | 57 | 1745 |
| | | | | | AGGCTC | | |
| 854487 | N/A | N/A | 12516 | 12531 | CGGCTTGGTT | 79 | 1746 |
| | | | | | TTGCCC | | |
| 854493 | N/A | N/A | 12522 | 12537 | CCGCCCCGGC | 78 | 1747 |
| | | | | | TTGGTT | | |
| 854499 | N/A | N/A | 12529 | 12544 | CCACGGGCCG | 93 | 1748 |
| | | | | | CCCCGG | | |
| 854505 | N/A | N/A | 12537 | 12552 | CTTGCTCCCC | 79 | 1749 |
| | | | | | ACGGGC | | |
| 854511 | N/A | N/A | 12563 | 12578 | TGGCCTCAAC | 67 | 1750 |
| | | | | | ACAAGC | | |
| 854517 | N/A | N/A | 15700 | 15715 | GACATGGGTC | 72 | 1751 |
| | | | | | AGGACT | | |
| 854523 | N/A | N/A | 15747 | 15762 | AAGCTGCACA | 100 | 1752 |
| | | | | | GAGTTC | | |
| 854529 | N/A | N/A | 17294 | 17309 | TCCACGGTTG | 57 | 1753 |
| | | | | | TCCCCA | | |
| 854535 | N/A | N/A | 17303 | 17318 | TTCCTAGTAT | 45 | 1754 |
| | | | | | CCACGG | | |
| 854541 | N/A | N/A | 17309 | 17324 | AAGGACTTCC | 85 | 1755 |
| | | | | | TAGTAT | | |
| 854547 | N/A | N/A | 17531 | 17546 | CGAGTATTCA | 30 | 1756 |
| | | | | | TAGACT | | |
| 854553 | N/A | N/A | 17539 | 17554 | AATGTGCTCG | 65 | 1757 |
| | | | | | AGTATT | | |
| 854559 | N/A | N/A | 18097 | 18112 | CTTACTCCTT | 53 | 1758 |
| | | | | | GACTCA | | |
| 854565 | N/A | N/A | 18115 | 18130 | TATGAGTTGG | 77 | 1759 |
| | | | | | TCCTGT | | |
| 854571 | N/A | N/A | 18122 | 18137 | AGGGTCCTAT | 71 | 1760 |
| | | | | | GAGTTG | | |
| 854577 | N/A | N/A | 18133 | 18148 | TGGTCTCTGA | 47 | 1761 |
| | | | | | CAGGGT | | |
| 854583 | N/A | N/A | 18435 | 18450 | TCATCCAGGC | 72 | 1762 |
| | | | | | CGCTGC | | |
| 854589 | N/A | N/A | 18496 | 18511 | TCCACCCTGC | 45 | 1763 |
| | | | | | CGCTGC | | |
| 854595 | N/A | N/A | 18537 | 18552 | TTCATTGGCA | 83 | 1764 |
| | | | | | GCCACC | | |
| 854601 | N/A | N/A | 18544 | 18559 | TCCCGGCTTC | 75 | 1765 |
| | | | | | ATTGGC | | |
| 854607 | N/A | N/A | 18550 | 18565 | GGCCAGTCCC | 73 | 1766 |
| | | | | | GGCTTC | | |
| 854613 | N/A | N/A | 20209 | 20224 | TCACTGCGCA | 79 | 1767 |
| | | | | | GACACT | | |

TABLE 24
Reduction of SPDEF RNA by 4 μM 3-10-3 cEt
gapmers with a phosphorothioate backbone

| | SEQ | SEQ | SEQ | SEQ | | | |
|---|---|---|---|---|---|---|---|
| | ID | ID | ID | ID | | | |
| | NO: | NO: | NO: | NO: | | SPD | |
| Com- | 1 | 1 | 2 | 2 | Sequence | EF | SEQ |
| pound | Start | Stop | Start | Stop | (5′ to | (% | ID |
| Number | Site | Site | Site | Site | 3′) | UTC) | NO |
| 652636 | N/A | N/A | 8821 | 8836 | GCCACATG | 65 | 1768 |
| | | | | | TCTGGATT | | |
| 801690 | 82 | 97 | 1749 | 1764 | CAGCAGGC | 81 | 174 |
| | | | | | TTGGAGGA | | |
| 802055 | N/A | N/A | 12531 | 12546 | CCCCACGG | 73 | 387 |
| | | | | | GCCGCCCC | | |
| 854165 | N/A | N/A | 2091 | 2106 | ATGCCAGG | 84 | 1769 |
| | | | | | GTACCCCC | | |
| 854171 | N/A | N/A | 2130 | 2145 | CTGCAGTC | 69 | 1770 |
| | | | | | GCCCACCC | | |
| 854177 | N/A | N/A | 2148 | 2163 | GCCCCCTC | 71 | 1771 |
| | | | | | CAAGTCCT | | |
| 854183 | N/A | N/A | 2154 | 2169 | AAGTGCGC | 46 | 1772 |
| | | | | | CCCCTCCA | | |
| 854189 | N/A | N/A | 2354 | 2369 | GGCGGCCT | 69 | 1773 |
| | | | | | CCCCTCAG | | |
| 854195 | N/A | N/A | 2363 | 2378 | CATCTGCA | 73 | 1774 |
| | | | | | CGGCGGCC | | |
| 854201 | N/A | N/A | 3363 | 3378 | CCCCACCA | 78 | 1775 |
| | | | | | TTTGTCTG | | |
| 854207 | N/A | N/A | 3381 | 3396 | GGGAGCTC | 99 | 1776 |
| | | | | | CGAGAATG | | |
| 854213 | N/A | N/A | 3684 | 3699 | GTTTTTGC | 55 | 1777 |
| | | | | | ACTTCCTG | | |
| 854219 | N/A | N/A | 3690 | 3705 | CTCCCGGT | 80 | 1778 |
| | | | | | TTTTGCAC | | |
| 854225 | N/A | N/A | 3696 | 3711 | TGGGTTCT | 95 | 1779 |
| | | | | | CCCGGTTT | | |
| 854231 | N/A | N/A | 3704 | 3719 | GTAAAAAG | 76 | 1780 |
| | | | | | GTGGTTCT | | |
| 854237 | N/A | N/A | 3721 | 3736 | TCACAACG | 68 | 1781 |
| | | | | | ACCTCAGT | | |
| 854243 | N/A | N/A | 5758 | 5773 | TACCTCCC | 77 | 1782 |
| | | | | | AGCTTGCC | | |
| 854249 | N/A | N/A | 5765 | 5780 | TACGCCGT | 69 | 1783 |
| | | | | | ACCTCCCA | | |
| 854255 | N/A | N/A | 5774 | 5789 | AGCCCCGC | 36 | 1784 |
| | | | | | ATACGCCG | | |
| 854261 | N/A | N/A | 5809 | 5824 | GGCCACAG | 69 | 1785 |
| | | | | | TACCTTCC | | |
| 854267 | N/A | N/A | 6299 | 6314 | GGAGTTGA | 109 | 1786 |
| | | | | | TGTCTGGA | | |
| 854273 | N/A | N/A | 6305 | 6320 | GTCCCTGG | 91 | 1787 |
| | | | | | AGTTGATG | | |
| 854279 | N/A | N/A | 7404 | 7419 | TCCGGGCT | 75 | 1788 |
| | | | | | TTCCCCAC | | |
| 854285 | N/A | N/A | 7412 | 7427 | TCTAGATC | 55 | 1789 |
| | | | | | TCCGGGCT | | |
| 854291 | N/A | N/A | 7426 | 7441 | CGAGGTGG | 74 | 1790 |
| | | | | | GTTTCTTC | | |
| 854297 | N/A | N/A | 7436 | 7451 | CATGAGTA | 76 | 1791 |
| | | | | | GACGAGGT | | |
| 854303 | N/A | N/A | 8813 | 8828 | TCTGGATT | 49 | 1792 |
| | | | | | AAGGCTCA | | |
| 854314 | N/A | N/A | 8832 | 8847 | TTCTGGAT | 53 | 1793 |
| | | | | | TAAGCCAC | | |
| 854320 | N/A | N/A | 8847 | 8862 | CCTCGAGT | 56 | 1794 |
| | | | | | TGATCTCT | | |
| 854326 | N/A | N/A | 8854 | 8869 | TCCCAAAC | 81 | 1795 |
| | | | | | CTCGAGTT | | |
| 854332 | N/A | N/A | 9120 | 9135 | ATCAGTGA | 71 | 1796 |
| | | | | | TGGTCCAC | | |
| 854338 | N/A | N/A | 9151 | 9166 | GTGCCATG | 47 | 1797 |
| | | | | | CAATTCAG | | |
| 854344 | N/A | N/A | 9848 | 9863 | ACCCGCTT | 63 | 1798 |
| | | | | | TCCTACCC | | |
| 854350 | N/A | N/A | 9854 | 9869 | CACATCAC | 86 | 1799 |
| | | | | | CCGCTTTC | | |
| 854356 | N/A | N/A | 9867 | 9882 | CCAGGCTT | 66 | 1800 |
| | | | | | TCACCCAC | | |
| 854362 | N/A | N/A | 9876 | 9891 | GTGCCACG | 86 | 1801 |
| | | | | | CCCAGGCT | | |
| 854368 | N/A | N/A | 9887 | 9902 | AGTGAGCA | 89 | 1802 |
| | | | | | CCAGTGCC | | |
| 854374 | N/A | N/A | 9913 | 9928 | AGAGAACG | 72 | 1803 |
| | | | | | GCACTGTG | | |
| 854380 | N/A | N/A | 9970 | 9985 | TGCCCAAC | 97 | 1804 |
| | | | | | CTGCAACT | | |
| 854386 | N/A | N/A | 9980 | 9995 | AGGGCTGG | 92 | 1805 |
| | | | | | TGTGCCCA | | |
| 854392 | N/A | N/A | 10003 | 10018 | CTTGGCTT | 70 | 1806 |
| | | | | | ACTGGTCA | | |
| 854398 | N/A | N/A | 10144 | 10159 | CGGTCCTA | 48 | 1807 |
| | | | | | GCTCCAAC | | |
| 854404 | N/A | N/A | 10150 | 10165 | AAGCCTCG | 64 | 1808 |
| | | | | | GTCCTAGC | | |
| 854410 | N/A | N/A | 10166 | 10181 | GAATCTAC | 74 | 1809 |
| | | | | | TCCCCACC | | |
| 854416 | N/A | N/A | 10173 | 10188 | TGCCAAGG | 64 | 1810 |
| | | | | | AATCTACT | | |
| 854422 | N/A | N/A | 10188 | 10203 | TGCCCTAT | 88 | 1811 |
| | | | | | ACCTAAAT | | |
| 854428 | N/A | N/A | 11238 | 11253 | GCTCCTTT | 87 | 1812 |
| | | | | | AAGTGACA | | |
| 854434 | N/A | N/A | 11251 | 11266 | CTCCCATT | 82 | 1813 |
| | | | | | CAAGGGCT | | |
| 854440 | N/A | N/A | 11278 | 11293 | GGAAGGGT | 109 | 1814 |
| | | | | | GTTCCCTT | | |
| 854446 | N/A | N/A | 11601 | 11616 | GGGCTCCC | 80 | 1815 |
| | | | | | TGATCCAT | | |
| 854452 | N/A | N/A | 11633 | 11648 | TGGTGCCC | 45 | 1816 |
| | | | | | TACTGGGA | | |
| 854458 | N/A | N/A | 11639 | 11654 | CATTTATG | 49 | 1817 |
| | | | | | GTGCCCTA | | |
| 854464 | N/A | N/A | 11655 | 11670 | TCCTCTCT | 98 | 1818 |
| | | | | | AACAGTGA | | |
| 854470 | N/A | N/A | 12003 | 12018 | AGATATAC | 67 | 1819 |
| | | | | | GCTCCTAA | | |
| 854476 | N/A | N/A | 12017 | 12032 | AGAAGATT | 56 | 1820 |
| | | | | | ACCAACAG | | |
| 854482 | N/A | N/A | 12370 | 12385 | TACATCAA | 53 | 1821 |
| | | | | | GACAGGCT | | |
| 854488 | N/A | N/A | 12517 | 12532 | CCGGCTTG | 78 | 1822 |
| | | | | | GTTTTGCC | | |
| 854494 | N/A | N/A | 12523 | 12538 | GCCGCCCC | 91 | 1823 |
| | | | | | GGCTTGGT | | |
| 854500 | N/A | N/A | 12530 | 12545 | CCCACGGG | 64 | 1824 |
| | | | | | CCGCCCCG | | |
| 854506 | N/A | N/A | 12538 | 12553 | CCTTGCTC | 71 | 1825 |
| | | | | | CCCACGGG | | |
| 854512 | N/A | N/A | 12564 | 12579 | CTGGCCTC | 67 | 1826 |
| | | | | | AACACAAG | | |
| 854518 | N/A | N/A | 15732 | 15747 | CAGTGCTG | 106 | 1827 |
| | | | | | CAATGCCA | | |
| 854524 | N/A | N/A | 17265 | 17280 | GCATCCTC | 53 | 1828 |
| | | | | | ACAGTCTG | | |
| 854530 | N/A | N/A | 17296 | 17311 | TATCCACG | 60 | 1829 |
| | | | | | GTTGTCCC | | |
| 854536 | N/A | N/A | 17304 | 17319 | CTTCCTAG | 56 | 1830 |
| | | | | | TATCCACG | | |
| 854542 | N/A | N/A | 17490 | 17505 | TTGTAACA | 55 | 1831 |
| | | | | | GTGGTTCC | | |
| 854548 | N/A | N/A | 17532 | 17547 | TCGAGTAT | 56 | 1832 |
| | | | | | TCATAGAC | | |
| 854554 | N/A | N/A | 17540 | 17555 | AAATGTGC | 72 | 1833 |
| | | | | | TCGAGTAT | | |
| 854560 | N/A | N/A | 18098 | 18113 | TCTTACTC | 72 | 1834 |
| | | | | | CTTGACTC | | |
| 854566 | N/A | N/A | 18116 | 18131 | CTATGAGT | 87 | 1835 |
| | | | | | TGGTCCTG | | |
| 854572 | N/A | N/A | 18123 | 18138 | CAGGGTCC | 68 | 1836 |
| | | | | | TATGAGTT | | |
| 854578 | N/A | N/A | 18134 | 18149 | CTGGTCTC | 71 | 1837 |
| | | | | | TGACAGGG | | |
| 854584 | N/A | N/A | 18473 | 18488 | ACCACAGT | 110 | 1838 |
| | | | | | AGTGAATC | | |
| 854590 | N/A | N/A | 18498 | 18513 | CCTCCACC | 92 | 1839 |
| | | | | | CTGCCGCT | | |
| 854596 | N/A | N/A | 18539 | 18554 | GCTTCATT | 104 | 1840 |
| | | | | | GGCAGCCA | | |
| 854602 | N/A | N/A | 18545 | 18560 | GTCCCGGC | 103 | 1841 |
| | | | | | TTCATTGG | | |
| 854608 | N/A | N/A | 20185 | 20200 | GTCAGTTC | 45 | 1842 |
| | | | | | TCTAGTAT | | |
| 854614 | N/A | N/A | 20210 | 20225 | TTCACTGC | 73 | 1843 |
| | | | | | GCAGACAC | | |

TABLE 25
Reduction of SPDEF RNA by 4 μM 3-10-3 cEt
gapmers with a phosphorothioate backbone

| | SEQ | SEQ | SEQ | SEQ | | | |
|---|---|---|---|---|---|---|---|
| | ID | ID | ID | ID | | | |
| | NO: | NO: | NO: | NO: | | | |
| Com- | 1 | 1 | 2 | 2 | Sequence | SPDEF | SEQ |
| pound | Start | Stop | Start | Stop | (5′ to | (% | ID |
| Number | Site | Site | Site | Site | 3′) | UTC) | NO |
| 801690 | 82 | 97 | 1749 | 1764 | CAGCAGGC | 87 | 174 |
| | | | | | TTGGAGGA | | |
| 802055 | N/A | N/A | 12531 | 12546 | CCCCACGG | 66 | 387 |
| | | | | | GCCGCCCC | | |
| 854166 | N/A | N/A | 2092 | 2107 | CATGCCAG | 103 | 1844 |
| | | | | | GGTACCCC | | |
| 854172 | N/A | N/A | 2131 | 2146 | CCTGCAGT | 66 | 1845 |
| | | | | | CGCCCACC | | |
| 854178 | N/A | N/A | 2149 | 2164 | CGCCCCCT | 75 | 1846 |
| | | | | | CCAAGTCC | | |
| 854184 | N/A | N/A | 2155 | 2170 | AAAGTGCG | 61 | 1847 |
| | | | | | CCCCCTCC | | |
| 854190 | N/A | N/A | 2356 | 2371 | ACGGCGGC | 62 | 1848 |
| | | | | | CTCCCCTC | | |
| 854196 | N/A | N/A | 2364 | 2379 | GCATCTGC | 44 | 1849 |
| | | | | | ACGGCGGC | | |
| 854202 | N/A | N/A | 3372 | 3387 | GAGAATGC | 73 | 1850 |
| | | | | | CCCCCACC | | |
| 854208 | N/A | N/A | 3382 | 3397 | TGGGAGCT | 106 | 1851 |
| | | | | | CCGAGAAT | | |
| 854214 | N/A | N/A | 3685 | 3700 | GGTTTTTG | 51 | 1852 |
| | | | | | CACTTCCT | | |
| 854220 | N/A | N/A | 3691 | 3706 | TCTCCCGG | 80 | 1853 |
| | | | | | TTTTTGCA | | |
| 854226 | N/A | N/A | 3698 | 3713 | AGTGGGTT | 40 | 1854 |
| | | | | | CTCCCGGT | | |
| 854232 | N/A | N/A | 3715 | 3730 | CGACCTCA | 66 | 1855 |
| | | | | | GTGGTAAA | | |
| 854238 | N/A | N/A | 3723 | 3738 | ACTCACAA | 80 | 1856 |
| | | | | | CGACCTCA | | |
| 854244 | N/A | N/A | 5759 | 5774 | GTACCTCC | 94 | 1857 |
| | | | | | CAGCTTGC | | |
| 854250 | N/A | N/A | 5766 | 5781 | ATACGCCG | 65 | 1858 |
| | | | | | TACCTCCC | | |
| 854256 | N/A | N/A | 5775 | 5790 | CAGCCCCG | 63 | 1859 |
| | | | | | CATACGCC | | |
| 854262 | N/A | N/A | 6294 | 6309 | TGATGTCT | 66 | 1860 |
| | | | | | GGAGGCTC | | |
| 854268 | N/A | N/A | 6300 | 6315 | TGGAGTTG | 82 | 1861 |
| | | | | | ATGTCTGG | | |
| 854274 | N/A | N/A | 6306 | 6321 | TGTCCCTG | 110 | 1862 |
| | | | | | GAGTTGAT | | |
| 854280 | N/A | N/A | 7405 | 7420 | CTCCGGGC | 57 | 1863 |
| | | | | | TTTCCCCA | | |
| 854286 | N/A | N/A | 7413 | 7428 | TTCTAGAT | 83 | 1864 |
| | | | | | CTCCGGGC | | |
| 854292 | N/A | N/A | 7427 | 7442 | ACGAGGTG | 102 | 1865 |
| | | | | | GGTTTCTT | | |
| 854298 | N/A | N/A | 7438 | 7453 | AGCATGAG | 65 | 1866 |
| | | | | | TAGACGAG | | |
| 854304 | N/A | N/A | 8814 | 8829 | GTCTGGAT | 62 | 1867 |
| | | | | | TAAGGCTC | | |
| 854309 | N/A | N/A | 8822 | 8837 | AGCCACAT | 95 | 1868 |
| | | | | | GTCTGGAT | | |
| 854315 | N/A | N/A | 8840 | 8855 | TTGATCTC | 78 | 1869 |
| | | | | | TTCTGGAT | | |
| 854321 | N/A | N/A | 8848 | 8863 | ACCTCGAG | 62 | 1870 |
| | | | | | TTGATCTC | | |
| 854327 | N/A | N/A | 9113 | 9128 | ATGGTCCA | 99 | 1871 |
| | | | | | CCCATGGG | | |
| 854333 | N/A | N/A | 9121 | 9136 | CATCAGTG | 85 | 1872 |
| | | | | | ATGGTCCA | | |
| 854339 | N/A | N/A | 9152 | 9167 | TGTGCCAT | 56 | 1873 |
| | | | | | GCAATTCA | | |
| 854345 | N/A | N/A | 9849 | 9864 | CACCCGCT | 97 | 1874 |
| | | | | | TTCCTACC | | |
| 854351 | N/A | N/A | 9856 | 9871 | CCCACATC | 83 | 1875 |
| | | | | | ACCCGCTT | | |
| 854357 | N/A | N/A | 9869 | 9884 | GCCCAGGC | 107 | 1876 |
| | | | | | TTTCACCC | | |
| 854363 | N/A | N/A | 9880 | 9895 | ACCAGTGC | 56 | 1877 |
| | | | | | CACGCCCA | | |
| 854369 | N/A | N/A | 9888 | 9903 | CAGTGAGC | 75 | 1878 |
| | | | | | ACCAGTGC | | |
| 854375 | N/A | N/A | 9947 | 9962 | TCAAGGTT | 85 | 1879 |
| | | | | | CTGGGCTG | | |
| 854381 | N/A | N/A | 9975 | 9990 | TGGTGTGC | 109 | 1880 |
| | | | | | CCAACCTG | | |
| 854387 | N/A | N/A | 9997 | 10012 | TTACTGGT | 71 | 1881 |
| | | | | | CAGGCAGC | | |
| 854393 | N/A | N/A | 10004 | 10019 | GCTTGGCT | 51 | 1882 |
| | | | | | TACTGGTC | | |
| 854399 | N/A | N/A | 10145 | 10160 | TCGGTCCT | 67 | 1883 |
| | | | | | AGCTCCAA | | |
| 854405 | N/A | N/A | 10151 | 10166 | CAAGCCTC | 64 | 1884 |
| | | | | | GGTCCTAG | | |
| 854411 | N/A | N/A | 10167 | 10182 | GGAATCTA | 78 | 1885 |
| | | | | | CTCCCCAC | | |
| 854417 | N/A | N/A | 10181 | 10196 | TACCTAAA | 81 | 1886 |
| | | | | | TGCCAAGG | | |
| 854423 | N/A | N/A | 10189 | 10204 | TTGCCCTA | 82 | 1887 |
| | | | | | TACCTAAA | | |
| 854429 | N/A | N/A | 11239 | 11254 | GGCTCCTT | 120 | 1888 |
| | | | | | TAAGTGAC | | |
| 854435 | N/A | N/A | 11252 | 11267 | TCTCCCAT | 108 | 1889 |
| | | | | | TCAAGGGC | | |
| 854441 | N/A | N/A | 11593 | 11608 | TGATCCAT | 101 | 1890 |
| | | | | | CTCCAGTT | | |
| 854447 | N/A | N/A | 11627 | 11642 | CCTACTGG | 63 | 1891 |
| | | | | | GACAGCAG | | |
| 854453 | N/A | N/A | 11634 | 11649 | ATGGTGCC | 47 | 1892 |
| | | | | | CTACTGGG | | |
| 854459 | N/A | N/A | 11641 | 11656 | GACATTTA | 34 | 1893 |
| | | | | | TGGTGCCC | | |
| 854465 | N/A | N/A | 11997 | 12012 | ACGCTCCT | 91 | 1894 |
| | | | | | AATAATAC | | |
| 854471 | N/A | N/A | 12004 | 12019 | CAGATATA | 50 | 1895 |
| | | | | | CGCTCCTA | | |
| 854477 | N/A | N/A | 12018 | 12033 | CAGAAGAT | 69 | 1896 |
| | | | | | TACCAACA | | |
| 854483 | N/A | N/A | 12388 | 12403 | GGGCCTGA | 109 | 1897 |
| | | | | | GACTTAAG | | |
| 854489 | N/A | N/A | 12518 | 12533 | CCCGGCTT | 90 | 1898 |
| | | | | | GGTTTTGC | | |
| 854495 | N/A | N/A | 12525 | 12540 | GGGCCGCC | 103 | 1899 |
| | | | | | CCGGCTTG | | |
| 854501 | N/A | N/A | 12532 | 12547 | TCCCCACG | 60 | 1900 |
| | | | | | GGCCGCCC | | |
| 854507 | N/A | N/A | 12539 | 12554 | GCCTTGCT | 108 | 1901 |
| | | | | | CCCCACGG | | |
| 854513 | N/A | N/A | 12567 | 12582 | CATCTGGC | 109 | 1902 |
| | | | | | CTCAACAC | | |
| 854519 | N/A | N/A | 15733 | 15748 | TCAGTGCT | 53 | 1903 |
| | | | | | GCAATGCC | | |
| 854525 | N/A | N/A | 17275 | 17290 | CTGACATC | 94 | 1904 |
| | | | | | CTGCATCC | | |
| 854531 | N/A | N/A | 17298 | 17313 | AGTATCCA | 58 | 1905 |
| | | | | | CGGTTGTC | | |
| 854537 | N/A | N/A | 17305 | 17320 | ACTTCCTA | 55 | 1906 |
| | | | | | GTATCCAC | | |
| 854543 | N/A | N/A | 17491 | 17506 | CTTGTAAC | 61 | 1907 |
| | | | | | AGTGGTTC | | |
| 854549 | N/A | N/A | 17534 | 17549 | GCTCGAGT | 68 | 1908 |
| | | | | | ATTCATAG | | |
| 854555 | N/A | N/A | 17541 | 17556 | TAAATGTG | 79 | 1909 |
| | | | | | CTCGAGTA | | |
| 854561 | N/A | N/A | 18099 | 18114 | TTCTTACT | 78 | 1910 |
| | | | | | CCTTGACT | | |
| 854567 | N/A | N/A | 18117 | 18132 | CCTATGAG | 74 | 1911 |
| | | | | | TTGGTCCT | | |
| 854573 | N/A | N/A | 18124 | 18139 | ACAGGGTC | 81 | 1912 |
| | | | | | CTATGAGT | | |
| 854579 | N/A | N/A | 18135 | 18150 | ACTGGTCT | 72 | 1913 |
| | | | | | CTGACAGG | | |
| 854585 | N/A | N/A | 18474 | 18489 | CACCACAG | 110 | 1914 |
| | | | | | TAGTGAAT | | |
| 854591 | N/A | N/A | 18513 | 18528 | CCACCCGA | 72 | 1915 |
| | | | | | GCCCCCGC | | |
| 854597 | N/A | N/A | 18540 | 18555 | GGCTTCAT | 102 | 1916 |
| | | | | | TGGCAGCC | | |
| 854603 | N/A | N/A | 18546 | 18561 | AGTCCCGG | 109 | 1917 |
| | | | | | CTTCATTG | | |
| 854609 | N/A | N/A | 20186 | 20201 | TGTCAGTT | 82 | 1918 |
| | | | | | CTCTAGTA | | |
| 854615 | N/A | N/A | 20212 | 20227 | CATTCACT | 83 | 1919 |
| | | | | | GCGCAGAC | | |

TABLE 26
Reduction of SPDEF RNA by 4 μM 3-10-3 cEt
gapmers with a phosphorothioate backbone

| | SEQ | SEQ | SEQ | SEQ | | | |
|---|---|---|---|---|---|---|---|
| | ID | ID | ID | ID | | | |
| | NO: | NO: | NO: | NO: | | | |
| Com- | 1 | 1 | 2 | 2 | Sequence | SPDEF | SEQ |
| pound | Start | Stop | Start | Stop | (5′ to | (% | ID |
| Number | Site | Site | Site | Site | 3′) | UTC) | NO |
| 801690 | 82 | 97 | 1749 | 1764 | CAGCAGGC | 95 | 174 |
| | | | | | TTGGAGGA | | |
| 802055 | N/A | N/A | 12531 | 12546 | CCCCACGG | 63 | 387 |
| | | | | | GCCGCCCC | | |
| 854167 | N/A | N/A | 2093 | 2108 | CCATGCCA | 81 | 1920 |
| | | | | | GGGTACCC | | |
| 854173 | N/A | N/A | 2132 | 2147 | GCCTGCAG | 78 | 1921 |
| | | | | | TCGCCCAC | | |
| 854179 | N/A | N/A | 2150 | 2165 | GCGCCCCC | 100 | 1922 |
| | | | | | TCCAAGTC | | |
| 854185 | N/A | N/A | 2156 | 2171 | CAAAGTGC | 78 | 1923 |
| | | | | | GCCCCCTC | | |
| 854191 | N/A | N/A | 2357 | 2372 | CACGGCGG | 62 | 1924 |
| | | | | | CCTCCCCT | | |
| 854197 | N/A | N/A | 2367 | 2382 | TCTGCATC | 69 | 1925 |
| | | | | | TGCACGGC | | |
| 854203 | N/A | N/A | 3377 | 3392 | GCTCCGAG | 94 | 1926 |
| | | | | | AATGCCCC | | |
| 854209 | N/A | N/A | 3383 | 3398 | CTGGGAGC | 92 | 1927 |
| | | | | | TCCGAGAA | | |
| 854215 | N/A | N/A | 3686 | 3701 | CGGTTTTT | 32 | 1928 |
| | | | | | GCACTTCC | | |
| 854221 | N/A | N/A | 3692 | 3707 | TTCTCCCG | 90 | 1929 |
| | | | | | GTTTTTGC | | |
| 854227 | N/A | N/A | 3699 | 3714 | AAGTGGGT | 51 | 1930 |
| | | | | | TCTCCCGG | | |
| 854233 | N/A | N/A | 3716 | 3731 | ACGACCTC | 56 | 1931 |
| | | | | | AGTGGTAA | | |
| 854239 | N/A | N/A | 3724 | 3739 | TACTCACA | 69 | 1932 |
| | | | | | ACGACCTC | | |
| 854245 | N/A | N/A | 5760 | 5775 | CGTACCTC | 90 | 1933 |
| | | | | | CCAGCTTG | | |
| 854251 | N/A | N/A | 5767 | 5782 | CATACGCC | 91 | 1934 |
| | | | | | GTACCTCC | | |
| 854257 | N/A | N/A | 5776 | 5791 | CCAGCCCC | 75 | 1935 |
| | | | | | GCATACGC | | |
| 854263 | N/A | N/A | 6295 | 6310 | TTGATGTC | 79 | 1936 |
| | | | | | TGGAGGCT | | |
| 854269 | N/A | N/A | 6301 | 6316 | CTGGAGTT | 88 | 1937 |
| | | | | | GATGTCTG | | |
| 854275 | N/A | N/A | 7372 | 7387 | ACTGTCCA | 68 | 1938 |
| | | | | | GGCCAACT | | |
| 854281 | N/A | N/A | 7407 | 7422 | ATCTCCGG | 75 | 1939 |
| | | | | | GCTTTCCC | | |
| 854287 | N/A | N/A | 7414 | 7429 | CTTCTAGA | 87 | 1940 |
| | | | | | TCTCCGGG | | |
| 854293 | N/A | N/A | 7429 | 7444 | AGACGAGG | 104 | 1941 |
| | | | | | TGGGTTTC | | |
| 854299 | N/A | N/A | 7440 | 7455 | TCAGCATG | 90 | 1942 |
| | | | | | AGTAGACG | | |
| 854305 | N/A | N/A | 8815 | 8830 | TGTCTGGA | 89 | 1943 |
| | | | | | TTAAGGCT | | |
| 854310 | N/A | N/A | 8827 | 8842 | GATTAAGC | 77 | 1944 |
| | | | | | CACATGTC | | |
| 854316 | N/A | N/A | 8841 | 8856 | GTTGATCT | 59 | 1945 |
| | | | | | CTTCTGGA | | |
| 854322 | N/A | N/A | 8849 | 8864 | AACCTCGA | 99 | 1946 |
| | | | | | GTTGATCT | | |
| 854328 | N/A | N/A | 9114 | 9129 | GATGGTCC | 84 | 1947 |
| | | | | | ACCCATGG | | |
| 854334 | N/A | N/A | 9122 | 9137 | CCATCAGT | 78 | 1948 |
| | | | | | GATGGTCC | | |
| 854340 | N/A | N/A | 9153 | 9168 | CTGTGCCA | 53 | 1949 |
| | | | | | TGCAATTC | | |
| 854346 | N/A | N/A | 9850 | 9865 | TCACCCGC | 80 | 1950 |
| | | | | | TTTCCTAC | | |
| 854352 | N/A | N/A | 9857 | 9872 | ACCCACAT | 84 | 1951 |
| | | | | | CACCCGCT | | |
| 854358 | N/A | N/A | 9870 | 9885 | CGCCCAGG | 81 | 1952 |
| | | | | | CTTTCACC | | |
| 854364 | N/A | N/A | 9882 | 9897 | GCACCAGT | 95 | 1953 |
| | | | | | GCCACGCC | | |
| 854370 | N/A | N/A | 9908 | 9923 | ACGGCACT | 95 | 1954 |
| | | | | | GTGAGCCC | | |
| 854376 | N/A | N/A | 9965 | 9980 | AACCTGCA | 50 | 1955 |
| | | | | | ACTAGGCG | | |
| 854382 | N/A | N/A | 9976 | 9991 | CTGGTGTG | 96 | 1956 |
| | | | | | CCCAACCT | | |
| 854388 | N/A | N/A | 9998 | 10013 | CTTACTGG | 77 | 1957 |
| | | | | | TCAGGCAG | | |
| 854394 | N/A | N/A | 10005 | 10020 | GGCTTGGC | 67 | 1958 |
| | | | | | TTACTGGT | | |
| 854400 | N/A | N/A | 10146 | 10161 | CTCGGTCC | 69 | 1959 |
| | | | | | TAGCTCCA | | |
| 854406 | N/A | N/A | 10152 | 10167 | CCAAGCCT | 69 | 1960 |
| | | | | | CGGTCCTA | | |
| 854412 | N/A | N/A | 10168 | 10183 | AGGAATCT | 84 | 1961 |
| | | | | | ACTCCCCA | | |
| 854418 | N/A | N/A | 10182 | 10197 | ATACCTAA | 90 | 1962 |
| | | | | | ATGCCAAG | | |
| 854424 | N/A | N/A | 10190 | 10205 | CTTGCCCT | 84 | 1963 |
| | | | | | ATACCTAA | | |
| 854430 | N/A | N/A | 11240 | 11255 | GGGCTCCT | 96 | 1964 |
| | | | | | TTAAGTGA | | |
| 854436 | N/A | N/A | 11274 | 11289 | GGGTGTTC | 86 | 1965 |
| | | | | | CCTTTGAT | | |
| 854442 | N/A | N/A | 11594 | 11609 | CTGATCCA | 102 | 1966 |
| | | | | | TCTCCAGT | | |
| 854448 | N/A | N/A | 11629 | 11644 | GCCCTACT | 77 | 1967 |
| | | | | | GGGACAGC | | |
| 854454 | N/A | N/A | 11635 | 11650 | TATGGTGC | 67 | 1968 |
| | | | | | CCTACTGG | | |
| 854460 | N/A | N/A | 11643 | 11658 | GTGACATT | 65 | 1969 |
| | | | | | TATGGTGC | | |
| 854466 | N/A | N/A | 11999 | 12014 | ATACGCTC | 102 | 1970 |
| | | | | | CTAATAAT | | |
| 854472 | N/A | N/A | 12006 | 12021 | AACAGATA | 47 | 1971 |
| | | | | | TACGCTCC | | |
| 854478 | N/A | N/A | 12020 | 12035 | GGCAGAAG | 72 | 1972 |
| | | | | | ATTACCAA | | |
| 854484 | N/A | N/A | 12500 | 12515 | AGGCCCTT | 98 | 1973 |
| | | | | | TTCCCTGA | | |
| 854490 | N/A | N/A | 12519 | 12534 | CCCCGGCT | 87 | 1974 |
| | | | | | TGGTTTTG | | |
| 854496 | N/A | N/A | 12526 | 12541 | CGGGCCGC | 83 | 1975 |
| | | | | | CCCGGCTT | | |
| 854502 | N/A | N/A | 12534 | 12549 | GCTCCCCA | 65 | 1976 |
| | | | | | CGGGCCGC | | |
| 854508 | N/A | N/A | 12548 | 12563 | CAGTCAGA | 86 | 1977 |
| | | | | | GGCCTTGC | | |
| 854514 | N/A | N/A | 15697 | 15712 | ATGGGTCA | 84 | 1978 |
| | | | | | GGACTGCC | | |
| 854520 | N/A | N/A | 15734 | 15749 | TTCAGTGC | 71 | 1979 |
| | | | | | TGCAATGC | | |
| 854526 | N/A | N/A | 17289 | 17304 | GGTTGTCC | 50 | 1980 |
| | | | | | CCAGCTCT | | |
| 854532 | N/A | N/A | 17299 | 17314 | TAGTATCC | 84 | 1981 |
| | | | | | ACGGTTGT | | |
| 854538 | N/A | N/A | 17306 | 17321 | GACTTCCT | 45 | 1982 |
| | | | | | AGTATCCA | | |
| 854544 | N/A | N/A | 17492 | 17507 | ACTTGTAA | 46 | 1983 |
| | | | | | CAGTGGTT | | |
| 854550 | N/A | N/A | 17536 | 17551 | GTGCTCGA | 59 | 1984 |
| | | | | | GTATTCAT | | |
| 854556 | N/A | N/A | 17543 | 17558 | TGTAAATG | 65 | 1985 |
| | | | | | TGCTCGAG | | |
| 854562 | N/A | N/A | 18112 | 18127 | GAGTTGGT | 88 | 1986 |
| | | | | | CCTGTTTC | | |
| 854568 | N/A | N/A | 18119 | 18134 | GTCCTATG | 93 | 1987 |
| | | | | | AGTTGGTC | | |
| 854574 | N/A | N/A | 18125 | 18140 | GACAGGGT | 85 | 1988 |
| | | | | | CCTATGAG | | |
| 854580 | N/A | N/A | 18136 | 18151 | CACTGGTC | 79 | 1989 |
| | | | | | TCTGACAG | | |
| 854586 | N/A | N/A | 18475 | 18490 | TCACCACA | 121 | 1990 |
| | | | | | GTAGTGAA | | |
| 854592 | N/A | N/A | 18534 | 18549 | ATTGGCAG | 106 | 1991 |
| | | | | | CCACCCCT | | |
| 854598 | N/A | N/A | 18541 | 18556 | CGGCTTCA | 105 | 1992 |
| | | | | | TTGGCAGC | | |
| 854604 | N/A | N/A | 18547 | 18562 | CAGTCCCG | 115 | 1993 |
| | | | | | GCTTCATT | | |
| 854610 | N/A | N/A | 20206 | 20221 | CTGCGCAG | 80 | 1994 |
| | | | | | ACACTGGG | | |
| 854616 | N/A | N/A | 20213 | 20228 | CCATTCAC | 69 | 1995 |
| | | | | | TGCGCAGA | | |

TABLE 27
Reduction of SPDEF RNA by 4 μM 3-10-3 cEt
gapmers with a phosphorothioate backbone

| | SEQ | SEQ | SEQ | SEQ | | | |
|---|---|---|---|---|---|---|---|
| | ID | ID | ID | ID | | | |
| | NO: | NO: | NO: | NO: | | | |
| Com- | 1 | 1 | 2 | 2 | Sequence | SPDEF | SEQ |
| pound | Start | Stop | Start | Stop | (5′ to | (% | ID |
| Number | Site | Site | Site | Site | 3′) | UTC) | NO |
| 801690 | 82 | 97 | 1749 | 1764 | CAGCAGGC | 80 | 174 |
| | | | | | TTGGAGGA | | |
| 802055 | N/A | N/A | 12531 | 12546 | CCCCACGG | 104 | 387 |
| | | | | | GCCGCCCC | | |
| 854168 | N/A | N/A | 2101 | 2116 | GAAAGACC | 108 | 1996 |
| | | | | | CCATGCCA | | |
| 854174 | N/A | N/A | 2133 | 2148 | TGCCTGCA | 107 | 1997 |
| | | | | | GTCGCCCA | | |
| 854180 | N/A | N/A | 2151 | 2166 | TGCGCCCC | 80 | 1998 |
| | | | | | CTCCAAGT | | |
| 854186 | N/A | N/A | 2157 | 2172 | GCAAAGTG | 68 | 1999 |
| | | | | | CGCCCCCT | | |
| 854192 | N/A | N/A | 2360 | 2375 | CTGCACGG | 80 | 2000 |
| | | | | | CGGCCTCC | | |
| 854198 | N/A | N/A | 2368 | 2383 | CTCTGCAT | 67 | 2001 |
| | | | | | CTGCACGG | | |
| 854204 | N/A | N/A | 3378 | 3393 | AGCTCCGA | 65 | 2002 |
| | | | | | GAATGCCC | | |
| 854210 | N/A | N/A | 3384 | 3399 | CCTGGGAG | 83 | 2003 |
| | | | | | CTCCGAGA | | |
| 854216 | N/A | N/A | 3687 | 3702 | CCGGTTTT | 52 | 2004 |
| | | | | | TGCACTTC | | |
| 854222 | N/A | N/A | 3693 | 3708 | GTTCTCCC | 116 | 2005 |
| | | | | | GGTTTTTG | | |
| 854228 | N/A | N/A | 3700 | 3715 | AAAGTGGG | 71 | 2006 |
| | | | | | TTCTCCCG | | |
| 854234 | N/A | N/A | 3717 | 3732 | AACGACCT | 60 | 2007 |
| | | | | | CAGTGGTA | | |
| 854240 | N/A | N/A | 3725 | 3740 | ATACTCAC | 67 | 2008 |
| | | | | | AACGACCT | | |
| 854246 | N/A | N/A | 5761 | 5776 | CCGTACCT | 89 | 2009 |
| | | | | | CCCAGCTT | | |
| 854252 | N/A | N/A | 5769 | 5784 | CGCATACG | 63 | 2010 |
| | | | | | CCGTACCT | | |
| 854258 | N/A | N/A | 5777 | 5792 | TCCAGCCC | 86 | 2011 |
| | | | | | CGCATACG | | |
| 854264 | N/A | N/A | 6296 | 6311 | GTTGATGT | 76 | 2012 |
| | | | | | CTGGAGGC | | |
| 854270 | N/A | N/A | 6302 | 6317 | CCTGGAGT | 89 | 2013 |
| | | | | | TGATGTCT | | |
| 854276 | N/A | N/A | 7373 | 7388 | AACTGTCC | 81 | 2014 |
| | | | | | AGGCCAAC | | |
| 854282 | N/A | N/A | 7409 | 7424 | AGATCTCC | 68 | 2015 |
| | | | | | GGGCTTTC | | |
| 854288 | N/A | N/A | 7415 | 7430 | TCTTCTAG | 57 | 2016 |
| | | | | | ATCTCCGG | | |
| 854294 | N/A | N/A | 7430 | 7445 | TAGACGAG | 104 | 2017 |
| | | | | | GTGGGTTT | | |
| 854300 | N/A | N/A | 7441 | 7456 | CTCAGCAT | 86 | 2018 |
| | | | | | GAGTAGAC | | |
| 854306 | N/A | N/A | 8816 | 8831 | ATGTCTGG | 80 | 2019 |
| | | | | | ATTAAGGC | | |
| 854311 | N/A | N/A | 8829 | 8844 | TGGATTAA | 88 | 2020 |
| | | | | | GCCACATG | | |
| 854317 | N/A | N/A | 8843 | 8858 | GAGTTGAT | 83 | 2021 |
| | | | | | CTCTTCTG | | |
| 854323 | N/A | N/A | 8850 | 8865 | AAACCTCG | 101 | 2022 |
| | | | | | AGTTGATC | | |
| 854329 | N/A | N/A | 9115 | 9130 | TGATGGTC | 90 | 2023 |
| | | | | | CACCCATG | | |
| 854335 | N/A | N/A | 9138 | 9153 | CAGCCCAG | 83 | 2024 |
| | | | | | GATTAAAT | | |
| 854341 | N/A | N/A | 9154 | 9169 | TCTGTGCC | 74 | 2025 |
| | | | | | ATGCAATT | | |
| 854347 | N/A | N/A | 9851 | 9866 | ATCACCCG | 79 | 2026 |
| | | | | | CTTTCCTA | | |
| 854353 | N/A | N/A | 9864 | 9879 | GGCTTTCA | 66 | 2027 |
| | | | | | CCCACATC | | |
| 854359 | N/A | N/A | 9871 | 9886 | ACGCCCAG | 76 | 2028 |
| | | | | | GCTTTCAC | | |
| 854365 | N/A | N/A | 9883 | 9898 | AGCACCAG | 79 | 2029 |
| | | | | | TGCCACGC | | |
| 854371 | N/A | N/A | 9909 | 9924 | AACGGCAC | 93 | 2030 |
| | | | | | TGTGAGCC | | |
| 854377 | N/A | N/A | 9966 | 9981 | CAACCTGC | 95 | 2031 |
| | | | | | AACTAGGC | | |
| 854383 | N/A | N/A | 9977 | 9992 | GCTGGTGT | 70 | 2032 |
| | | | | | GCCCAACC | | |
| 854389 | N/A | N/A | 9999 | 10014 | GCTTACTG | 92 | 2033 |
| | | | | | GTCAGGCA | | |
| 854395 | N/A | N/A | 10006 | 10021 | GGGCTTGG | 90 | 2034 |
| | | | | | CTTACTGG | | |
| 854401 | N/A | N/A | 10147 | 10162 | CCTCGGTC | 76 | 2035 |
| | | | | | CTAGCTCC | | |
| 854407 | N/A | N/A | 10153 | 10168 | ACCAAGCC | 89 | 2036 |
| | | | | | TCGGTCCT | | |
| 854413 | N/A | N/A | 10169 | 10184 | AAGGAATC | 67 | 2037 |
| | | | | | TACTCCCC | | |
| 854419 | N/A | N/A | 10183 | 10198 | TATACCTA | 94 | 2038 |
| | | | | | AATGCCAA | | |
| 854425 | N/A | N/A | 10191 | 10206 | CCTTGCCC | 81 | 2039 |
| | | | | | TATACCTA | | |
| 854431 | N/A | N/A | 11241 | 11256 | AGGGCTCC | 71 | 2040 |
| | | | | | TTTAAGTG | | |
| 854437 | N/A | N/A | 11275 | 11290 | AGGGTGTT | 86 | 2041 |
| | | | | | CCCTTTGA | | |
| 854443 | N/A | N/A | 11595 | 11610 | CCTGATCC | 82 | 2042 |
| | | | | | ATCTCCAG | | |
| 854449 | N/A | N/A | 11630 | 11645 | TGCCCTAC | 102 | 2043 |
| | | | | | TGGGACAG | | |
| 854455 | N/A | N/A | 11636 | 11651 | TTATGGTG | 88 | 2044 |
| | | | | | CCCTACTG | | |
| 854461 | N/A | N/A | 11651 | 11666 | CTCTAACA | 99 | 2045 |
| | | | | | GTGACATT | | |
| 854467 | N/A | N/A | 12000 | 12015 | TATACGCT | 84 | 2046 |
| | | | | | CCTAATAA | | |
| 854473 | N/A | N/A | 12007 | 12022 | CAACAGAT | 94 | 2047 |
| | | | | | ATACGCTC | | |
| 854479 | N/A | N/A | 12021 | 12036 | AGGCAGAA | 87 | 2048 |
| | | | | | GATTACCA | | |
| 854485 | N/A | N/A | 12514 | 12529 | GCTTGGTT | 86 | 2049 |
| | | | | | TTGCCCAG | | |
| 854491 | N/A | N/A | 12520 | 12535 | GCCCCGGC | 92 | 2050 |
| | | | | | TTGGTTTT | | |
| 854497 | N/A | N/A | 12527 | 12542 | ACGGGCCG | 96 | 2051 |
| | | | | | CCCCGGCT | | |
| 854503 | N/A | N/A | 12535 | 12550 | TGCTCCCC | 102 | 2052 |
| | | | | | ACGGGCCG | | |
| 854509 | N/A | N/A | 12559 | 12574 | CTCAACAC | 106 | 2053 |
| | | | | | AAGCAGTC | | |
| 854515 | N/A | N/A | 15698 | 15713 | CATGGGTC | 92 | 2054 |
| | | | | | AGGACTGC | | |
| 854521 | N/A | N/A | 15745 | 15760 | GCTGCACA | 86 | 2055 |
| | | | | | GAGTTCAG | | |
| 854527 | N/A | N/A | 17291 | 17306 | ACGGTTGT | 57 | 2056 |
| | | | | | CCCCAGCT | | |
| 854533 | N/A | N/A | 17300 | 17315 | CTAGTATC | 75 | 2057 |
| | | | | | CACGGTTG | | |
| 854539 | N/A | N/A | 17307 | 17322 | GGACTTCC | 83 | 2058 |
| | | | | | TAGTATCC | | |
| 854545 | N/A | N/A | 17493 | 17508 | AACTTGTA | 43 | 2059 |
| | | | | | ACAGTGGT | | |
| 854551 | N/A | N/A | 17537 | 17552 | TGTGCTCG | 72 | 2060 |
| | | | | | AGTATTCA | | |
| 854557 | N/A | N/A | 17544 | 17559 | ATGTAAAT | 72 | 2061 |
| | | | | | GTGCTCGA | | |
| 854563 | N/A | N/A | 18113 | 18128 | TGAGTTGG | 88 | 2062 |
| | | | | | TCCTGTTT | | |
| 854569 | N/A | N/A | 18120 | 18135 | GGTCCTAT | 71 | 2063 |
| | | | | | GAGTTGGT | | |
| 854575 | N/A | N/A | 18129 | 18144 | CTCTGACA | 62 | 2064 |
| | | | | | GGGTCCTA | | |
| 854581 | N/A | N/A | 18433 | 18448 | ATCCAGGC | 114 | 2065 |
| | | | | | CGCTGCAG | | |
| 854587 | N/A | N/A | 18476 | 18491 | CTCACCAC | 88 | 2066 |
| | | | | | AGTAGTGA | | |
| 854593 | N/A | N/A | 18535 | 18550 | CATTGGCA | 97 | 2067 |
| | | | | | GCCACCCC | | |
| 854599 | N/A | N/A | 18542 | 18557 | CCGGCTTC | 81 | 2068 |
| | | | | | ATTGGCAG | | |
| 854605 | N/A | N/A | 18548 | 18563 | CCAGTCCC | 99 | 2069 |
| | | | | | GGCTTCAT | | |
| 854611 | N/A | N/A | 20207 | 20222 | ACTGCGCA | 92 | 2070 |
| | | | | | GACACTGG | | |
| 854617 | N/A | N/A | 20214 | 20229 | GCCATTCA | 101 | 2071 |
| | | | | | CTGCGCAG | | |

TABLE 28
Reduction of SPDEF RNA by 4 μM 3-10-3 cEt
gapmers with a phosphorothioate backbone

| | SEQ | SEQ | SEQ | SEQ | | | |
|---|---|---|---|---|---|---|---|
| | ID | ID | ID | ID | | | |
| | NO: | NO: | NO: | NO: | | | |
| Com- | 1 | 1 | 2 | 2 | Sequence | SPDEF | SEQ |
| pound | Start | Stop | Start | Stop | (5′ to | (% | ID |
| Number | Site | Site | Site | Site | 3′) | UTC) | NO |
| 801690 | 82 | 97 | 1749 | 1764 | CAGCAGGC | 113 | 174 |
| | | | | | TTGGAGGA | | |
| 802055 | N/A | N/A | 12531 | 12546 | CCCCACGG | 77 | 387 |
| | | | | | GCCGCCCC | | |
| 854169 | N/A | N/A | 2119 | 2134 | CACCCCCC | 91 | 2072 |
| | | | | | AGCTGGCA | | |
| 854175 | N/A | N/A | 2134 | 2149 | CTGCCTGC | 107 | 2073 |
| | | | | | AGTCGCCC | | |
| 854181 | N/A | N/A | 2152 | 2167 | GTGCGCCC | 90 | 2074 |
| | | | | | CCTCCAAG | | |
| 854187 | N/A | N/A | 2159 | 2174 | TGGCAAAG | 108 | 2075 |
| | | | | | TGCGCCCC | | |
| 854193 | N/A | N/A | 2361 | 2376 | TCTGCACG | 73 | 2076 |
| | | | | | GCGGCCTC | | |
| 854199 | N/A | N/A | 3361 | 3376 | CCACCATT | 96 | 2077 |
| | | | | | TGTCTGTG | | |
| 854205 | N/A | N/A | 3379 | 3394 | GAGCTCCG | 88 | 2078 |
| | | | | | AGAATGCC | | |
| 854211 | N/A | N/A | 3385 | 3400 | GCCTGGGA | 65 | 2079 |
| | | | | | GCTCCGAG | | |
| 854217 | N/A | N/A | 3688 | 3703 | CCCGGTTT | 100 | 2080 |
| | | | | | TTGCACTT | | |
| 854223 | N/A | N/A | 3694 | 3709 | GGTTCTCC | 81 | 2081 |
| | | | | | CGGTTTTT | | |
| 854229 | N/A | N/A | 3701 | 3716 | AAAAGTGG | 84 | 2082 |
| | | | | | GTTCTCCC | | |
| 854235 | N/A | N/A | 3719 | 3734 | ACAACGAC | 55 | 2083 |
| | | | | | CTCAGTGG | | |
| 854241 | N/A | N/A | 3727 | 3742 | TTATACTC | 78 | 2084 |
| | | | | | ACAACGAC | | |
| 854247 | N/A | N/A | 5762 | 5777 | GCCGTACC | 112 | 2085 |
| | | | | | TCCCAGCT | | |
| 854253 | N/A | N/A | 5770 | 5785 | CCGCATAC | 70 | 2086 |
| | | | | | GCCGTACC | | |
| 854259 | N/A | N/A | 5804 | 5819 | CAGTACCT | 100 | 2087 |
| | | | | | TCCCTCTT | | |
| 854265 | N/A | N/A | 6297 | 6312 | AGTTGATG | 89 | 2088 |
| | | | | | TCTGGAGG | | |
| 854271 | N/A | N/A | 6303 | 6318 | CCCTGGAG | 120 | 2089 |
| | | | | | TTGATGTC | | |
| 854277 | N/A | N/A | 7374 | 7389 | AAACTGTC | 78 | 2090 |
| | | | | | CAGGCCAA | | |
| 854283 | N/A | N/A | 7410 | 7425 | TAGATCTC | 71 | 2091 |
| | | | | | CGGGCTTT | | |
| 854289 | N/A | N/A | 7416 | 7431 | TTCTTCTA | 80 | 2092 |
| | | | | | GATCTCCG | | |
| 854295 | N/A | N/A | 7434 | 7449 | TGAGTAGA | 100 | 2093 |
| | | | | | CGAGGTGG | | |
| 854301 | N/A | N/A | 7442 | 7457 | ACTCAGCA | 82 | 2094 |
| | | | | | TGAGTAGA | | |
| 854307 | N/A | N/A | 8819 | 8834 | CACATGTC | 90 | 2095 |
| | | | | | TGGATTAA | | |
| 854312 | N/A | N/A | 8830 | 8845 | CTGGATTA | 83 | 2096 |
| | | | | | AGCCACAT | | |
| 854318 | N/A | N/A | 8844 | 8859 | CGAGTTGA | 99 | 2097 |
| | | | | | TCTCTTCT | | |
| 854324 | N/A | N/A | 8851 | 8866 | CAAACCTC | 82 | 2098 |
| | | | | | GAGTTGAT | | |
| 854330 | N/A | N/A | 9116 | 9131 | GTGATGGT | 84 | 2099 |
| | | | | | CCACCCAT | | |
| 854336 | N/A | N/A | 9139 | 9154 | TCAGCCCA | 80 | 2100 |
| | | | | | GGATTAAA | | |
| 854342 | N/A | N/A | 9846 | 9861 | CCGCTTTC | 83 | 2101 |
| | | | | | CTACCCAC | | |
| 854348 | N/A | N/A | 9852 | 9867 | CATCACCC | 123 | 2102 |
| | | | | | GCTTTCCT | | |
| 854354 | N/A | N/A | 9865 | 9880 | AGGCTTTC | 99 | 2103 |
| | | | | | ACCCACAT | | |
| 854360 | N/A | N/A | 9872 | 9887 | CACGCCCA | 64 | 2104 |
| | | | | | GGCTTTCA | | |
| 854366 | N/A | N/A | 9884 | 9899 | GAGCACCA | 81 | 2105 |
| | | | | | GTGCCACG | | |
| 854372 | N/A | N/A | 9910 | 9925 | GAACGGCA | 112 | 2106 |
| | | | | | CTGTGAGC | | |
| 854378 | N/A | N/A | 9967 | 9982 | CCAACCTG | 101 | 2107 |
| | | | | | CAACTAGG | | |
| 854384 | N/A | N/A | 9978 | 9993 | GGCTGGTG | 87 | 2108 |
| | | | | | TGCCCAAC | | |
| 854390 | N/A | N/A | 10001 | 10016 | TGGCTTAC | 68 | 2109 |
| | | | | | TGGTCAGG | | |
| 854396 | N/A | N/A | 10142 | 10157 | GTCCTAGC | 81 | 2110 |
| | | | | | TCCAACAC | | |
| 854402 | N/A | N/A | 10148 | 10163 | GCCTCGGT | 78 | 2111 |
| | | | | | CCTAGCTC | | |
| 854408 | N/A | N/A | 10155 | 10170 | CCACCAAG | 83 | 2112 |
| | | | | | CCTCGGTC | | |
| 854414 | N/A | N/A | 10170 | 10185 | CAAGGAAT | 86 | 2113 |
| | | | | | CTACTCCC | | |
| 854420 | N/A | N/A | 10185 | 10200 | CCTATACC | 92 | 2114 |
| | | | | | TAAATGCC | | |
| 854426 | N/A | N/A | 10192 | 10207 | ACCTTGCC | 91 | 2115 |
| | | | | | CTATACCT | | |
| 854432 | N/A | N/A | 11248 | 11263 | CCATTCAA | 99 | 2116 |
| | | | | | GGGCTCCT | | |
| 854438 | N/A | N/A | 11276 | 11291 | AAGGGTGT | 108 | 2117 |
| | | | | | TCCCTTTG | | |
| 854444 | N/A | N/A | 11599 | 11614 | GCTCCCTG | 76 | 2118 |
| | | | | | ATCCATCT | | |
| 854450 | N/A | N/A | 11631 | 11646 | GTGCCCTA | 104 | 2119 |
| | | | | | CTGGGACA | | |
| 854456 | N/A | N/A | 11637 | 11652 | TTTATGGT | 74 | 2120 |
| | | | | | GCCCTACT | | |
| 854462 | N/A | N/A | 11652 | 11667 | TCTCTAAC | 85 | 2121 |
| | | | | | AGTGACAT | | |
| 854468 | N/A | N/A | 12001 | 12016 | ATATACGC | 107 | 2122 |
| | | | | | TCCTAATA | | |
| 854474 | N/A | N/A | 12008 | 12023 | CCAACAGA | 75 | 2123 |
| | | | | | TATACGCT | | |
| 854480 | N/A | N/A | 12368 | 12383 | CATCAAGA | 95 | 2124 |
| | | | | | CAGGCTCA | | |
| 854486 | N/A | N/A | 12515 | 12530 | GGCTTGGT | 56 | 2125 |
| | | | | | TTTGCCCA | | |
| 854492 | N/A | N/A | 12521 | 12536 | CGCCCCGG | 70 | 2126 |
| | | | | | CTTGGTTT | | |
| 854498 | N/A | N/A | 12528 | 12543 | CACGGGCC | 77 | 2127 |
| | | | | | GCCCCGGC | | |
| 854504 | N/A | N/A | 12536 | 12551 | TTGCTCCC | 80 | 2128 |
| | | | | | CACGGGCC | | |
| 854510 | N/A | N/A | 12561 | 12576 | GCCTCAAC | 115 | 2129 |
| | | | | | ACAAGCAG | | |
| 854516 | N/A | N/A | 15699 | 15714 | ACATGGGT | 102 | 2130 |
| | | | | | CAGGACTG | | |
| 854522 | N/A | N/A | 15746 | 15761 | AGCTGCAC | 96 | 2131 |
| | | | | | AGAGTTCA | | |
| 854528 | N/A | N/A | 17293 | 17308 | CCACGGTT | 100 | 2132 |
| | | | | | GTCCCCAG | | |
| 854534 | N/A | N/A | 17301 | 17316 | CCTAGTAT | 89 | 2133 |
| | | | | | CCACGGTT | | |
| 854540 | N/A | N/A | 17308 | 17323 | AGGACTTC | 92 | 2134 |
| | | | | | CTAGTATC | | |
| 854546 | N/A | N/A | 17523 | 17538 | CATAGACT | 83 | 2135 |
| | | | | | TTCCCTGG | | |
| 854552 | N/A | N/A | 17538 | 17553 | ATGTGCTC | 89 | 2136 |
| | | | | | GAGTATTC | | |
| 854558 | N/A | N/A | 18096 | 18111 | TTACTCCT | 100 | 2137 |
| | | | | | TGACTCAG | | |
| 854564 | N/A | N/A | 18114 | 18129 | ATGAGTTG | 96 | 2138 |
| | | | | | GTCCTGTT | | |
| 854570 | N/A | N/A | 18121 | 18136 | GGGTCCTA | 105 | 2139 |
| | | | | | TGAGTTGG | | |
| 854576 | N/A | N/A | 18130 | 18145 | TCTCTGAC | 71 | 2140 |
| | | | | | AGGGTCCT | | |
| 854582 | N/A | N/A | 18434 | 18449 | CATCCAGG | 87 | 2141 |
| | | | | | CCGCTGCA | | |
| 854588 | N/A | N/A | 18478 | 18493 | GGCTCACC | 98 | 2142 |
| | | | | | ACAGTAGT | | |
| 854594 | N/A | N/A | 18536 | 18551 | TCATTGGC | 72 | 2143 |
| | | | | | AGCCACCC | | |
| 854600 | N/A | N/A | 18543 | 18558 | CCCGGCTT | 95 | 2144 |
| | | | | | CATTGGCA | | |
| 854606 | N/A | N/A | 18549 | 18564 | GCCAGTCC | 105 | 2145 |
| | | | | | CGGCTTCA | | |
| 854612 | N/A | N/A | 20208 | 20223 | CACTGCGC | 96 | 2146 |
| | | | | | AGACACTG | | |
| 854618 | N/A | N/A | 20216 | 20231 | GTGCCATT | 117 | 2147 |
| | | | | | CACTGCGC | | |

TABLE 29
Reduction of SPDEF RNA by 4 μM 3-10-3 cEt
gapmers with a phosphorothioate backbone

| | SEQ | SEQ | SEQ | SEQ | SEQ | SEQ | | | |
|---|---|---|---|---|---|---|---|---|---|
| | ID | ID | ID | ID | ID | ID | | | |
| | NO: | NO: | NO: | NO: | NO: | NO: | Se- | SPD | |
| Com- | 3 | 3 | 4 | 4 | 5 | 5 | quence | EF | SEQ |
| pound | Start | Stop | Start | Stop | Start | Stop | (5′ to | (% | ID |
| Number | Site | Site | Site | Site | Site | Site | 3′) | UTC) | NO |
| 801921 | 1055 | 1070 | N/A | N/A | N/A | N/A | GGCTGA | 98 | 2148 |
| | | | | | | | CTTCCA | | |
| | | | | | | | GATG | | |
| 801922 | 1060 | 1075 | N/A | N/A | N/A | N/A | GTCGAG | 86 | 2149 |
| | | | | | | | GCTGAC | | |
| | | | | | | | TTCC | | |
| 801923 | 1065 | 1080 | N/A | N/A | N/A | N/A | CACTGG | 101 | 2150 |
| | | | | | | | TCGAGG | | |
| | | | | | | | CTGA | | |
| 833205 | 1059 | 1074 | N/A | N/A | N/A | N/A | TCGAGG | 107 | 2151 |
| | | | | | | | CTGACT | | |
| | | | | | | | TCCA | | |
| 833206 | 1061 | 1076 | N/A | N/A | N/A | N/A | GGTCGA | 155 | 2152 |
| | | | | | | | GGCTGA | | |
| | | | | | | | CTTC | | |
| 833207 | 1062 | 1077 | N/A | N/A | N/A | N/A | TGGTCG | 124 | 2153 |
| | | | | | | | AGGCTG | | |
| | | | | | | | ACTT | | |
| 833208 | 1063 | 1078 | N/A | N/A | N/A | N/A | CTGGTC | 111 | 2154 |
| | | | | | | | GAGGCT | | |
| | | | | | | | GACT | | |
| 833209 | 1064 | 1079 | N/A | N/A | N/A | N/A | ACTGGT | 105 | 2155 |
| | | | | | | | CGAGGC | | |
| | | | | | | | TGAC | | |
| 833439 | N/A | N/A | 835 | 850 | N/A | N/A | TCTCCC | 60 | 2156 |
| | | | | | | | AGCTTG | | |
| | | | | | | | CCAC | | |
| 833440 | N/A | N/A | 836 | 851 | N/A | N/A | GTCTCC | 81 | 2157 |
| | | | | | | | CAGCTT | | |
| | | | | | | | GCCA | | |
| 833441 | N/A | N/A | 837 | 852 | N/A | N/A | TGTCTC | 87 | 2158 |
| | | | | | | | CCAGCT | | |
| | | | | | | | TGCC | | |
| 833442 | N/A | N/A | 845 | 860 | N/A | N/A | GGCGGC | 99 | 2159 |
| | | | | | | | TGTGTC | | |
| | | | | | | | TCCC | | |
| 833443 | N/A | N/A | 846 | 861 | N/A | N/A | TGGCGG | 143 | 2160 |
| | | | | | | | CTGTGT | | |
| | | | | | | | CTCC | | |
| 833444 | N/A | N/A | 847 | 862 | N/A | N/A | CTGGCG | 127 | 2161 |
| | | | | | | | GCTGTG | | |
| | | | | | | | TCTC | | |
| 833514 | N/A | N/A | N/A | N/A | 30 | 45 | GTCTGT | 107 | 2162 |
| | | | | | | | GAAGTG | | |
| | | | | | | | TCAG | | |
| 833515 | N/A | N/A | N/A | N/A | 31 | 46 | TGTCTG | 152 | 2163 |
| | | | | | | | TGAAGT | | |
| | | | | | | | GTCA | | |
| 833516 | N/A | N/A | N/A | N/A | 32 | 47 | GTGTCT | 100 | 2164 |
| | | | | | | | GTGAAG | | |
| | | | | | | | TGTC | | |
| 833517 | N/A | N/A | N/A | N/A | 39 | 54 | GGCGGC | 89 | 2165 |
| | | | | | | | TGTGTC | | |
| | | | | | | | TGTG | | |

### Example 2: Effect of Modified Oligonucleotides on Human SPDEF RNA In Vitro, Single Dose

Additional oligonucleotides with further chemistry modifications were designed to target an SPDEF nucleic acid and were tested for their effect on SPDEF RNA levels in vitro. The chemistry notation column in the tables below specifies the specific chemistry notation for modified oligonucleotides; wherein subscript ‘d’ represents a 2′-β-D-deoxyribosyl sugar moiety, subscript ‘e’ represents a 2′-MOE sugar moiety, subscript ‘y’ represents a 2′-O-methyl sugar moiety, subscript ‘k’ represents a cEt modified sugar moiety, subscript ‘s’ represents a phosphorothioate internucleoside linkage, and superscript ‘m’ before the cytosine residue represents a 5-methyl cytosine.

“Start site” indicates the 5′-most nucleoside to which the gapmer is targeted in the human gene sequence. “Stop site” indicates the 3′-most nucleoside to which the gapmer is targeted in the human gene sequence. Modified oligonucleotide listed in the tables below are targeted to either SEQ ID NO: 1 or SEQ ID NO: 2 (described herein above). ‘N/A’ indicates that the modified oligonucleotide does not target that particular gene sequence with 100% complementarity.

The modified oligonucleotides were tested in a series of experiments that had similar culture conditions. The results for each experiment are presented in separate tables shown below. Cultured VCaP cells at a density of 20,000 cells per well were transfected using electroporation with 4 μM of modified oligonucleotide. After a treatment period of approximately 24 hours, RNA was isolated from the cells and SPDEF RNA levels were measured by quantitative real-time RTPCR. Human primer probe set RTS35007 was used to measure RNA levels. SPDEF RNA levels were adjusted according to total RNA content, as measured by RIBOGREEN®. Reduction of SPDEF RNA is presented in the tables as percent SPDEF RNA levels relative to untreated control (UTC) cells (% UTC). Each table represents results from an individual assay plate. The compounds marked with an asterisk (*) indicate that the modified oligonucleotide is complementary to the amplicon region of the primer probe set. Additional assays may be used to measure the potency and efficacy of the modified oligonucleotides complementary to the amplicon region.

TABLE 30
Reduction of SPDEF RNA by 4 μM modified oligonucleotides

| | SEQ | SEQ | | | | |
|---|---|---|---|---|---|---|
| | ID | ID | | | | |
| Com- | NO: 2 | NO: 2 | | | SPDEF | SEQ |
| pound | Start | Stop | Sequence | Chemistry Notation | (% | ID |
| Number | Site | Site | (5′ to 3′) | (5′ to 3′) | UTC) | NO |
| 833814 | 12009 | 12024 | ACCAACAGATA | Aₖₛ^{m}Cₖₛ^{m}CₖₛA_{ds}A_{ds}^{m}C_{ds}A_{ds}G_{ds}A | 46 | 993 |
| | | | TACGC | _{ds}T_{ds}A_{ds}T_{ds}A_{ds}^{m}CₖₛGₖₛ^{m}Cₖ | | |
| 854302 | 8811 | 8826 | TGGATTAAGGC | TₖₛGₖₛGₖₛA_{ds}T_{ds}T_{ds}A_{ds}A_{ds}G_{ds}G_{ds} | 36 | 1715 |
| | | | TCAGC | ^{m}C_{ds}T_{ds}^{m}C_{ds}AₖₛGₖₛ^{m}Cₖ | | |
| 936288 | 3521 | 3536 | GTCTGGTAGTTT | GₖₛTₖₛ^{m}CₖₛT_{ds}G_{ds}G_{ds}T_{ds}A_{ds}G_{ds}T | 46 | 2166 |
| | | | TCAG | _{ds}T_{ds}T_{ds}T_{ds}^{m}CₖₛAₖₛGₖ | | |
| 936290 | 3523 | 3538 | AAGTCTGGTAG | AₖₛAₖₛGₖₛT_{ds}^{m}C_{ds}T_{ds}G_{ds}G_{ds}T_{ds}A | 47 | 2167 |
| | | | TTTTC | _{ds}G_{ds}T_{ds}T_{ds}TₖₛTₖₛ^{m}Cₖ | | |
| 936291 | 3524 | 3539 | CAAGTCTGGTA | ^{m}CₖₛAₖₛAₖₛG_{ds}T_{ds}^{m}C_{ds}T_{ds}G_{ds}G_{ds} | 48 | 2168 |
| | | | GTTTT | T_{ds}A_{ds}G_{ds}T_{ds}TₖₛTₖₛTₖ | | |
| 936292 | 3525 | 3540 | GCAAGTCTGGT | Gₖₛ^{m}CₖₛAₖₛA_{ds}G_{ds}T_{ds}^{m}C_{ds}T_{ds}G_{ds} | 38 | 2169 |
| | | | AGTTT | G_{ds}T_{ds}A_{ds}G_{ds}TₖₛTₖₛTₖ | | |
| 936293 | 3527 | 3542 | AAGCAAGTCTG | AₖₛAₖₛGₖₛ^{m}C_{ds}A_{ds}A_{ds}G_{ds}T_{ds}^{m}C_{ds} | 73 | 2170 |
| | | | GTAGT | T_{ds}G_{ds}G_{ds}T_{ds}AₖₛGₖₛTₖ | | |
| 936294 | 3528 | 3543 | TAAGCAAGTCT | TₖₛAₖₛAₖₛG_{ds}^{m}C_{ds}A_{ds}A_{ds}G_{ds}T_{ds}^{m} | 54 | 2171 |
| | | | GGTAG | C_{ds}T_{ds}G_{ds}G_{ds}TₖₛAₖₛGₖ | | |
| 936297 | 3535 | 3550 | TGTGCAATAAG | TₖₛGₖₛTₖₛG_{ds}^{m}C_{ds}A_{ds}A_{ds}T_{ds}A_{ds}A | 54 | 2172 |
| | | | CAAGT | _{ds}G_{ds}^{m}C_{ds}A_{ds}AₖₛGₖₛTₖ | | |
| 936298 | 3536 | 3551 | GTGTGCAATAA | GₖₛTₖₛGₖₛT_{ds}G_{ds}^{m}C_{ds}A_{ds}A_{ds}T_{ds}A | 45 | 2173 |
| | | | GCAAG | _{ds}A_{ds}G_{ds}^{m}C_{ds}AₖₛAₖₛGₖ | | |
| 936299 | 3537 | 3552 | AGTGTGCAATA | AₖₛGₖₛTₖₛG_{ds}T_{ds}G_{ds}^{m}C_{ds}A_{ds}A_{ds}T | 32 | 2174 |
| | | | AGCAA | _{ds}A_{ds}A_{ds}G_{ds}^{m}CₖₛAₖₛAₖ | | |
| 936300 | 3538 | 3553 | CAGTGTGCAAT | ^{m}CₖₛAₖₛGₖₛT_{ds}G_{ds}T_{ds}G_{ds}^{m}C_{ds}A_{ds} | 44 | 2175 |
| | | | AAGCA | A_{ds}T_{ds}A_{ds}A_{ds}Gₖₛ^{m}CₖₛAₖ | | |
| 936301 | 3539 | 3554 | ACAGTGTGCAA | Aₖₛ^{m}CₖₛAₖₛG_{ds}T_{ds}G_{ds}T_{ds}G_{ds}^{m}C_{ds} | 38 | 2176 |
| | | | TAAGC | A_{ds}A_{ds}T_{ds}A_{ds}AₖₛGₖₛ^{m}Cₖ | | |
| 936310 | 3785 | 3800 | CAGGCTGCAAC | ^{m}CₖₛAₖₛGₖₛG_{ds}^{m}C_{ds}T_{ds}G_{ds}^{m}C_{ds}A | 30 | 2177 |
| | | | AAGTC | _{ds}A_{ds}^{m}C_{ds}A_{ds}A_{ds}GₖₛTₖₛ^{m}Cₖ | | |
| 936311 | 3786 | 3801 | TCAGGCTGCAA | Tₖₛ^{m}CₖₛAₖₛG_{ds}G_{ds}^{m}C_{ds}T_{ds}G_{ds}^{m}C_{d} | 45 | 2178 |
| | | | CAAGT | sA_{ds}A_{ds}^{m}C_{ds}A_{ds}AₖₛGₖₛTₖ | | |
| 936312 | 3790 | 3805 | ATACTCAGGCT | AₖₛTₖₛAₖₛ^{m}C_{ds}T_{ds}^{m}C_{ds}A_{ds}G_{ds}G_{ds} | 60 | 2179 |
| | | | GCAAC | ^{m}C_{ds}T_{ds}G_{ds}^{m}C_{ds}AₖₛAₖₛ^{m}Cₖ | | |
| 936313 | 3791 | 3806 | TATACTCAGGC | TₖₛAₖₛTₖₛA_{ds}^{m}C_{ds}T_{ds}^{m}C_{ds}A_{ds}G_{ds} | 48 | 2180 |
| | | | TGCAA | G_{ds}^{m}C_{ds}T_{ds}G_{ds}^{m}CₖₛAₖₛAₖ | | |
| 936314 | 3792 | 3807 | TTATACTCAGG | TₖₛTₖₛAₖₛT_{ds}A_{ds}^{m}C_{ds}T_{ds}^{m}C_{ds}A_{ds} | 57 | 2181 |
| | | | CTGCA | G_{ds}G_{ds}^{m}C_{ds}T_{ds}Gₖₛ^{m}CₖₛAₖ | | |
| 936315 | 3794 | 3809 | GGTTATACTCA | GₖₛGₖₛTₖₛT_{ds}A_{ds}T_{ds}A_{ds}^{m}C_{ds}T_{ds}^{m} | 43 | 2182 |
| | | | GGCTG | C_{ds}A_{ds}G_{ds}G_{ds}^{m}CₖₛTₖₛGₖ | | |
| 936316 | 3796 | 3811 | CGGGTTATACT | ^{m}CₖₛGₖₛGₖₛG_{ds}T_{ds}T_{ds}A_{ds}T_{ds}A_{ds}^{m} | 35 | 2183 |
| | | | CAGGC | C_{ds}T_{ds}^{m}C_{ds}A_{ds}GₖₛGₖₛ^{m}Cₖ | | |
| 936317 | 3797 | 3812 | CCGGGTTATAC | ^{m}Cₖₛ^{m}CₖₛGₖₛG_{ds}G_{ds}T_{ds}T_{ds}A_{ds}T_{ds} | 43 | 2184 |
| | | | TCAGG | A_{ds}^{m}C_{ds}T_{ds}^{m}C_{ds}AₖₛGₖₛGₖ | | |
| 936318 | 3798 | 3813 | CCCGGGTTATA | ^{m}Cₖₛ^{m}Cₖₛ^{m}CₖₛG_{ds}G_{ds}G_{ds}T_{ds}T_{ds}A_{d} | 63 | 2185 |
| | | | CTCAG | ₛT_{ds}A_{ds}^{m}C_{ds}T_{ds}^{m}CₖₛAₖₛGₖ | | |
| 936325 | 3806 | 3821 | TTAACTTCCCCG | TₖₛTₖₛAₖₛA_{ds}^{m}C_{ds}T_{ds}T_{ds}^{m}C_{ds}^{m}C_{ds} | 52 | 2186 |
| | | | GGTT | ^{m}C_{ds}^{m}C_{ds}G_{ds}G_{ds}GₖₛTₖₛTₖ | | |
| 936326 | 3808 | 3823 | AATTAACTTCC | AₖₛAₖₛTₖₛT_{ds}A_{ds}A_{ds}^{m}C_{ds}T_{ds}T_{ds}^{m} | 84 | 2187 |
| | | | CCGGG | C_{ds}^{m}C_{ds}^{m}C_{ds}^{m}C_{ds}GₖₛGₖₛGₖ | | |
| 936327 | 3809 | 3824 | AAATTAACTTC | AₖₛAₖₛAₖₛT_{ds}T_{ds}A_{ds}A_{ds}^{m}C_{ds}T_{ds}T | 88 | 2188 |
| | | | CCCGG | _{ds}^{m}C_{ds}^{m}C_{ds}^{m}C_{ds}^{m}CₖₛGₖₛGₖ | | |
| 936329 | 6063 | 6078 | ATTCCGCTCAA | AₖₛTₖₛTₖₛ^{m}C_{ds}^{m}C_{ds}G_{ds}^{m}C_{ds}T_{ds}^{m}C | 64 | 2189 |
| | | | CCTTC | _{ds}A_{ds}A_{ds}^{m}C_{ds}^{m}C_{ds}TₖₛTₖₛ^{m}Cₖ | | |
| 936330 | 6064 | 6079 | AATTCCGCTCA | AₖₛAₖₛTₖₛT_{ds}^{m}C_{ds}^{m}C_{ds}G_{ds}^{m}C_{ds}T_{d} | 80 | 2190 |
| | | | ACCTT | ₛ^{m}C_{ds}A_{ds}A_{ds}^{m}C_{ds}^{m}CₖₛTₖₛTₖ | | |
| 936331 | 6065 | 6080 | CAATTCCGCTC | ^{m}CₖₛAₖₛAₖₛT_{ds}T_{ds}^{m}C_{ds}^{m}C_{ds}G_{ds}^{m} | 66 | 2191 |
| | | | AACCT | C_{ds}T_{ds}^{m}C_{ds}A_{ds}A_{ds}^{m}Cₖₛ^{m}CₖₛTₖ | | |
| 936332 | 6066 | 6081 | ACAATTCCGCT | Aₖₛ^{m}CₖₛAₖₛA_{ds}T_{ds}T_{ds}^{m}C_{ds}^{m}C_{ds}G_{d} | 71 | 2192 |
| | | | CAACC | ₛ^{m}C_{ds}T_{ds}^{m}C_{ds}A_{ds}Aₖₛ^{m}Cₖₛ^{m}Cₖ | | |
| 936333 | 6068 | 6083 | CTACAATTCCG | ^{m}CₖₛTₖₛAₖₛ^{m}C_{ds}A_{ds}A_{ds}T_{ds}T_{ds}^{m}C_{d} | 83 | 2193 |
| | | | CTCAA | ₛ^{m}C_{ds}G_{ds}^{m}C_{ds}T_{ds}^{m}CₖₛAₖₛAₖ | | |
| 936334 | 6070 | 6085 | AACTACAATTC | AₖₛAₖₛ^{m}CₖₛT_{ds}A_{ds}^{m}C_{ds}A_{ds}A_{ds}T_{ds} | 69 | 2194 |
| | | | CGCTC | T_{ds}^{m}C_{ds}^{m}C_{ds}G_{ds}^{m}CₖₛTₖₛ^{m}Cₖ | | |
| 936335 | 6071 | 6086 | CAACTACAATT | ^{m}CₖₛAₖₛAₖₛ^{m}C_{ds}T_{ds}A_{ds}^{m}C_{ds}A_{ds}A | 63 | 2195 |
| | | | CCGCT | _{ds}T_{ds}T_{ds}^{m}C_{ds}^{m}C_{ds}Gₖₛ^{m}CₖₛTₖ | | |
| 936336 | 6073 | 6088 | AGCAACTACAA | AₖₛGₖₛ^{m}CₖₛA_{ds}A_{ds}^{m}C_{ds}T_{ds}A_{ds}^{m}C | 27 | 2196 |
| | | | TTCCG | _{ds}A_{ds}A_{ds}T_{ds}T_{ds}^{m}Cₖₛ^{m}CₖₛGₖ | | |
| 936341 | 6081 | 6096 | CCCACCATAGC | ^{m}Cₖₛ^{m}Cₖₛ^{m}CₖₛA_{ds}^{m}C_{ds}^{m}C_{ds}A_{ds}T_{ds} | 50 | 2197 |
| | | | AACTA | A_{ds}G_{ds}^{m}C_{ds}A_{ds}A_{ds}^{m}CₖₛTₖₛAₖ | | |
| 936347 | 6356 | 6371 | AGGCATACTCC | AₖₛGₖₛGₖₛ^{m}C_{ds}A_{ds}T_{ds}A_{ds}^{m}C_{ds}T_{ds} | 55 | 2198 |
| | | | ATTTA | ^{m}C_{ds}^{m}C_{ds}A_{ds}T_{ds}TₖₛTₖₛAₖ | | |
| 936348 | 6357 | 6372 | AAGGCATACTC | AₖₛAₖₛGₖₛG_{ds}^{m}C_{ds}A_{ds}T_{ds}A_{ds}^{m}C_{ds} | 62 | 2199 |
| | | | CATTT | T_{ds}^{m}C_{ds}^{m}C_{ds}A_{ds}TₖₛTₖₛTₖ | | |
| 936349 | 6358 | 6373 | AAAGGCATACT | AₖₛAₖₛAₖₛG_{ds}G_{ds}^{m}C_{ds}A_{ds}T_{ds}A_{ds}^{m} | 53 | 2200 |
| | | | CCATT | C_{ds}T_{ds}^{m}C_{ds}^{m}C_{ds}AₖₛTₖₛTₖ | | |
| 936351 | 6370 | 6385 | TCCCTTGTAAG | Tₖₛ^{m}Cₖₛ^{m}Cₖₛ^{m}C_{ds}T_{ds}T_{ds}G_{ds}T_{ds}A_{ds} | 82 | 2201 |
| | | | CAAAG | A_{ds}G_{ds}^{m}C_{ds}A_{ds}AₖₛAₖₛGₖ | | |
| 936358 | 7446 | 7461 | TTGGACTCAGC | TₖₛTₖₛGₖₛG_{ds}A_{ds}^{m}C_{ds}T_{ds}^{m}C_{ds}A_{ds} | 63 | 2202 |
| | | | ATGAG | G_{ds}^{m}C_{ds}A_{ds}T_{ds}GₖₛAₖₛGₖ | | |
| 936359 | 7447 | 7462 | CTTGGACTCAG | ^{m}CₖₛTₖₛTₖₛG_{ds}G_{ds}A_{ds}^{m}C_{ds}T_{ds}^{m}C_{d} | 65 | 2203 |
| | | | CATGA | ₛA_{ds}G_{ds}^{m}C_{ds}A_{ds}TₖₛGₖₛAₖ | | |
| 936365 | 8290 | 8305 | TATCCTCACCCC | TₖₛAₖₛTₖₛ^{m}C_{ds}^{m}C_{ds}T_{ds}^{m}C_{ds}A_{ds}^{m}C | 68 | 2204 |
| | | | TACC | _{ds}^{m}C_{ds}^{m}C_{ds}^{m}C_{ds}T_{ds}Aₖₛ^{m}Cₖₛ^{m}Cₖ | | |
| 936368 | 8297 | 8312 | GTAAATGTATC | GₖₛTₖₛAₖₛA_{ds}A_{ds}T_{ds}G_{ds}T_{ds}A_{ds}T_{ds} | 77 | 2205 |
| | | | CTCAC | ^{m}C_{ds}^{m}C_{ds}T_{ds}^{m}CₖₛAₖₛ^{m}Cₖ | | |
| 936370 | 8579 | 8594 | TTAGTGCAGCT | TₖₛTₖₛAₖₛG_{ds}T_{ds}G_{ds}^{m}C_{ds}A_{ds}G_{ds}^{m} | 53 | 2206 |
| | | | TTTCC | C_{ds}T_{ds}T_{ds}T_{ds}Tₖₛ^{m}Cₖₛ^{m}Cₖ | | |
| 936376 | 8586 | 8601 | GAGAGATTTAG | GₖₛAₖₛGₖₛA_{ds}G_{ds}A_{ds}T_{ds}T_{ds}T_{ds}A_{ds} | 65 | 2207 |
| | | | TGCAG | G_{ds}T_{ds}G_{ds}^{m}CₖₛAₖₛGₖ | | |
| 936377 | 8588 | 8603 | AGGAGAGATTT | AₖₛGₖₛGₖₛA_{ds}G_{ds}A_{ds}G_{ds}A_{ds}T_{ds}T_{d} | 78 | 2208 |
| | | | AGTGC | ₛT_{ds}A_{ds}G_{ds}TₖₛGₖₛ^{m}Cₖ | | |
| 936378 | 9295 | 9310 | ATACCTGCCCC | AₖₛTₖₛAₖₛ^{m}C_{ds}^{m}C_{ds}T_{ds}G_{ds}^{m}C_{ds}^{m} | 62 | 2209 |
| | | | TGTGC | C_{ds}^{m}C_{ds}^{m}C_{ds}T_{ds}G_{ds}TₖₛGₖₛ^{m}Cₖ | | |
| 936379 | 9296 | 9311 | CATACCTGCCC | ^{m}CₖₛAₖₛTₖₛA_{ds}^{m}C_{ds}^{m}C_{ds}T_{ds}G_{ds}^{m} | 69 | 2210 |
| | | | CTGTG | C_{ds}^{m}C_{ds}^{m}C_{ds}^{m}C_{ds}T_{ds}GₖₛTₖₛGₖ | | |
| 936380 | 9297 | 9312 | TCATACCTGCC | Tₖₛ^{m}CₖₛAₖₛT_{ds}A_{ds}^{m}C_{ds}^{m}C_{ds}T_{ds}G_{d} | 70 | 2211 |
| | | | CCTGT | ₛ^{m}C_{ds}^{m}C_{ds}^{m}C_{ds}^{m}C_{ds}TₖₛGₖₛTₖ | | |
| 936381 | 9298 | 9313 | TTCATACCTGCC | TₖₛTₖₛ^{m}CₖₛA_{ds}T_{ds}A_{ds}^{m}C_{ds}^{m}C_{ds}T_{ds} | 43 | 2212 |
| | | | CCTG | G_{ds}^{m}C_{ds}^{m}C_{ds}^{m}C_{ds}^{m}CₖₛTₖₛGₖ | | |
| 936382 | 9300 | 9315 | ATTTCATACCTG | AₖₛTₖₛTₖₛT_{ds}^{m}C_{ds}A_{ds}T_{ds}A_{ds}^{m}C_{ds}^{m} | 46 | 2213 |
| | | | CCCC | C_{ds}T_{ds}G_{ds}^{m}C_{ds}^{m}Cₖₛ^{m}Cₖₛ^{m}Cₖ | | |
| 936383 | 9305 | 9320 | ATGGCATTTCA | AₖₛTₖₛGₖₛG_{ds}^{m}C_{ds}A_{ds}T_{ds}T_{ds}T_{ds}^{m} | 58 | 2214 |
| | | | TACCT | C_{ds}A_{ds}T_{ds}A_{ds}^{m}Cₖₛ^{m}CₖₛTₖ | | |
| 936389 | 9367 | 9382 | AGCAGGGTCCG | AₖₛGₖₛ^{m}CₖₛA_{ds}G_{ds}G_{ds}G_{ds}T_{ds}^{m}C_{ds} | 111 | 2215 |
| | | | GACCA | ^{m}C_{ds}G_{ds}G_{ds}A_{ds}^{m}Cₖ^{m}CₖₛAₖ | | |
| 936390 | 9369 | 9384 | GCAGCAGGGTC | Gₖₛ^{m}CₖₛAₖₛG_{ds}^{m}C_{ds}A_{ds}G_{ds}G_{ds}G_{ds} | 64 | 2216 |
| | | | CGGAC | T_{ds}^{m}C_{ds}^{m}C_{ds}G_{ds}GₖₛAₖₛ^{m}Cₖ | | |
| 936391 | 9370 | 9385 | AGCAGCAGGGT | AₖₛGₖₛ^{m}CₖₛA_{ds}G_{ds}^{m}C_{ds}A_{ds}G_{ds}G_{ds} | 62 | 2217 |
| | | | CCGGA | G_{ds}T_{ds}^{m}C_{ds}^{m}C_{ds}GₖₛGₖₛAₖ | | |
| 936392 | 9371 | 9386 | TAGCAGCAGGG | TₖₛAₖₛGₖₛ^{m}C_{ds}A_{ds}G_{ds}^{m}C_{ds}A_{ds}G_{ds} | 79 | 2218 |
| | | | TCCGG | G_{ds}G_{ds}T_{ds}^{m}C_{ds}^{m}CₖₛGₖₛGₖ | | |
| 936393 | 9372 | 9387 | TTAGCAGCAGG | TₖₛTₖₛAₖₛG_{ds}^{m}C_{ds}A_{ds}G_{ds}^{m}C_{ds}A_{ds} | 59 | 2219 |
| | | | GTCCG | G_{ds}G_{ds}G_{ds}T_{ds}^{m}Cₖₛ^{m}CₖₛGₖ | | |
| 936394 | 9373 | 9388 | ATTAGCAGCAG | AₖₛTₖₛTₖₛA_{ds}G_{ds}^{m}C_{ds}A_{ds}G_{ds}^{m}C_{ds} | 64 | 2220 |
| | | | GGTCC | A_{ds}G_{ds}G_{ds}G_{ds}Tₖₛ^{m}Cₖₛ^{m}Cₖ | | |
| 936396 | 9376 | 9391 | CTTATTAGCAG | ^{m}CₖₛTₖₛTₖₛA_{ds}T_{ds}T_{ds}A_{ds}G_{ds}^{m}C_{ds} | 38 | 2221 |
| | | | CAGGG | A_{ds}G_{ds}^{m}C_{ds}A_{ds}GₖₛGₖₛGₖ | | |
| 936397 | 9378 | 9393 | TGCTTATTAGC | TₖₛGₖₛ^{m}CₖₛT_{ds}T_{ds}A_{ds}T_{ds}T_{ds}A_{ds}G_{d} | 67 | 2222 |
| | | | AGCAG | ₛ^{m}C_{ds}A_{ds}G_{ds}^{m}CₖₛAₖₛGₖ | | |
| 936402 | 9791 | 9806 | TGCGGACAGTG | TₖₛGₖₛ^{m}CₖₛG_{ds}G_{ds}A_{ds}^{m}C_{ds}A_{ds}G_{ds} | 85 | 2223 |
| | | | AGGCT | T_{ds}G_{ds}A_{ds}G_{ds}Gₖₛ^{m}CₖₛTₖ | | |
| 936403 | 9792 | 9807 | ATGCGGACAGT | AₖₛTₖₛGₖₛ^{m}C_{ds}G_{ds}G_{ds}A_{ds}^{m}C_{ds}A_{ds} | 66 | 2224 |
| | | | GAGGC | G_{ds}T_{ds}G_{ds}A_{ds}GₖₛGₖₛ^{m}Cₖ | | |
| 936404 | 9793 | 9808 | GATGCGGACAG | GₖₛAₖₛTₖₛG_{ds}^{m}C_{ds}G_{ds}G_{ds}A_{ds}^{m}C_{ds} | 65 | 2225 |
| | | | TGAGG | A_{ds}G_{ds}T_{ds}G_{ds}AₖₛGₖₛGₖ | | |
| 936406 | 9795 | 9810 | TAGATGCGGAC | TₖₛAₖₛGₖₛA_{ds}T_{ds}G_{ds}^{m}C_{ds}G_{ds}G_{ds}A | 57 | 2226 |
| | | | AGTGA | _{ds}^{m}C_{ds}A_{ds}G_{ds}TₖₛGₖₛAₖ | | |
| 936407 | 9797 | 9812 | TATAGATGCGG | TₖₛAₖₛTₖₛA_{ds}G_{ds}A_{ds}T_{ds}G_{ds}^{m}C_{ds}G | 70 | 2227 |
| | | | ACAGT | _{ds}G_{ds}A_{ds}^{m}C_{ds}AₖₛGₖₛTₖ | | |
| 936408 | 9798 | 9813 | TTATAGATGCG | TₖₛTₖₛAₖₛT_{ds}A_{ds}G_{ds}A_{ds}T_{ds}G_{ds}^{m}C | 73 | 2228 |
| | | | GACAG | _{ds}G_{ds}G_{ds}A_{ds}^{m}CₖₛAₖₛGₖ | | |
| 936409 | 9800 | 9815 | CTTTATAGATG | ^{m}CₖₛTₖₛTₖₛT_{ds}A_{ds}T_{ds}A_{ds}G_{ds}A_{ds}T_{d} | 45 | 2229 |
| | | | CGGAC | ₛG_{ds}^{m}C_{ds}G_{ds}GₖₛAₖₛ^{m}Cₖ | | |
| 936410 | 9802 | 9817 | TGCTTTATAGAT | TₖₛGₖₛ^{m}CₖₛT_{ds}T_{ds}T_{ds}A_{ds}T_{ds}A_{ds}G_{d} | 48 | 2230 |
| | | | GCGG | ₛA_{ds}T_{ds}G_{ds}^{m}CₖₛGₖₛGₖ | | |
| 936412 | 9808 | 9823 | GAGCCCTGCTT | GₖₛAₖₛGₖₛ^{m}C_{ds}^{m}C_{ds}^{m}C_{ds}T_{ds}G_{ds}^{m} | 76 | 2231 |
| | | | TATAG | C_{ds}T_{ds}T_{ds}T_{ds}A_{ds}TₖₛAₖₛGₖ | | |
| 936413 | 10269 | 10284 | GAGGTAAATCC | GₖₛAₖₛGₖₛG_{ds}T_{ds}A_{ds}A_{ds}A_{ds}T_{ds}^{m}C | 44 | 2232 |
| | | | CCAAA | _{ds}^{m}C_{ds}^{m}C_{ds}^{m}C_{ds}AₖₛAₖₛAₖ | | |
| 936415 | 10271 | 10286 | GAGAGGTAAAT | GₖₛAₖₛGₖₛA_{ds}G_{ds}G_{ds}T_{ds}A_{ds}A_{ds}A_{d} | 33 | 2233 |
| | | | CCCCA | ₛT_{ds}^{m}C_{ds}^{m}C_{ds}^{m}Cₖₛ^{m}CₖₛAₖ | | |
| 936416 | 10273 | 10288 | CAGAGAGGTAA | ^{m}CₖₛAₖₛGₖₛA_{ds}G_{ds}A_{ds}G_{ds}G_{ds}T_{ds} | 28 | 2234 |
| | | | ATCCC | A_{ds}A_{ds}A_{ds}T_{ds}^{m}Cₖₛ^{m}Cₖ | | |
| 936419 | 10689 | 10704 | GACACATCCTT | GₖₛAₖₛ^{m}CₖₛA_{ds}^{m}C_{ds}A_{ds}T_{ds}^{m}C_{ds}^{m} | 45 | 2235 |
| | | | GACAC | C_{ds}T_{ds}T_{ds}G_{ds}A_{ds}^{m}CₖₛAₖₛ^{m}Cₖ | | |
| 936420 | 10691 | 10706 | ATGACACATCC | AₖₛTₖₛGₖₛA_{ds}^{m}C_{ds}A_{ds}^{m}C_{ds}A_{ds}T_{ds} | 61 | 2236 |
| | | | TTGAC | ^{m}C_{ds}^{m}C_{ds}T_{ds}T_{ds}GₖₛAₖₛ^{m}Cₖ | | |
| 936421 | 10693 | 10708 | TAATGACACAT | TₖₛAₖₛAₖₛT_{ds}G_{ds}A_{ds}^{m}C_{ds}A_{ds}^{m}C_{ds} | 38 | 2237 |
| | | | CCTTG | A_{ds}T_{ds}^{m}C_{ds}^{m}C_{ds}TₖₛTₖₛGₖ | | |
| 936422 | 10695 | 10710 | TATAATGACAC | TₖₛAₖₛTₖₛA_{ds}A_{ds}T_{ds}G_{ds}A_{ds}^{m}C_{ds}A | 47 | 2238 |
| | | | ATCCT | _{ds}^{m}C_{ds}A_{ds}T_{ds}^{m}Cₖₛ^{m}CₖₛTₖ | | |
| 936425 | 11995 | 12010 | GCTCCTAATAA | Gₖₛ^{m}CₖₛTₖₛ^{m}C_{ds}^{m}C_{ds}T_{ds}A_{ds}A_{ds}T_{d} | 68 | 2239 |
| | | | TACAG | sA_{ds}A_{ds}T_{ds}A_{ds}^{m}CₖₛAₖₛGₖ | | |
| 936426 | 11998 | 12013 | TACGCTCCTAA | TₖₛAₖₛ^{m}CₖₛG_{ds}^{m}C_{ds}T_{ds}^{m}C_{ds}^{m}C_{ds}T | 110 | 2240 |
| | | | TAATA | _{ds}A_{ds}A_{ds}T_{ds}A_{ds}AₖₛTₖₛAₖ | | |
| 936429 | 14110 | 14125 | TGTAGAAGTGC | TₖₛGₖₛTₖₛA_{ds}G_{ds}A_{ds}A_{ds}G_{ds}T_{ds}G_{ds} | 43 | 2241 |
| | | | CAGCA | ^{m}C_{ds}^{m}C_{ds}A_{ds}Gₖₛ^{m}CₖₛAₖ | | |

TABLE 31
Reduction of SPDEF RNA by 4 μM modified oligonucleotides

| | SEQ ID | SEQ ID | | | | |
|---|---|---|---|---|---|---|
| | NO: 2 | NO: 2 | | | SPDEF | |
| Compound | Start | Stop | Sequence | Chemistry Notation | (% | SEQ |
| Number | Site | Site | (5′ to 3′) | (5′ to 3′) | UTC) | ID NO |
| 833814 | 12009 | 12024 | ACCAACAGATA | Aₖₛ^{m}CₖₛmCₖₛA_{ds}A_{ds}^{m}C_{ds}A_{ds}G_{ds} | 39 | 993 |
| | | | TACGC | A_{ds}T_{ds}A_{ds}T_{ds}A_{ds}^{m}CₖₛGₖₛ^{m}Cₖ | | |
| 854302 | 8811 | 8826 | TGGATTAAGGC | TₖₛGₖₛGₖₛA_{ds}T_{ds}T_{ds}A_{ds}A_{ds}G_{ds} | 27 | 1715 |
| | | | TCAGC | G_{ds}^{m}C_{ds}T_{ds}mC_{ds}AₖₛGₖₛ^{m}Cₖ | | |
| 936068 | 3531 | 3546 | CAATAAGCAAG | ^{m}CₖₛAₖₛA_{ds}T_{ds}A_{ds}A_{ds}G_{ds}^{m}C_{ds} | 31 | 1129 |
| | | | TCTGG | A_{ds}A_{ds}G_{ds}Tₑₛ^{m}CₑₛTₑₛGₖₛGₖ | | |
| 936069 | 3685 | 3700 | GGTTTTTGCAC | GₖₛGₖₛT_{ds}T_{ds}T_{ds}T_{ds}T_{ds}G_{ds}^{m}C_{ds} | 29 | 1852 |
| | | | TTCCT | A_{ds}^{m}C_{ds}TₑₛTₑₛ^{m}Cₑₛ^{m}CₖₛTₖ | | |
| 936070 | 3795 | 3810 | GGGTTATACTC | GₖₛGₖₛG_{ds}T_{ds}T_{ds}A_{ds}T_{ds}A_{ds}^{m}C_{ds} | 20 | 1358 |
| | | | AGGCT | T_{ds}mC_{ds}AₑₛGₑₛGₑₛ^{m}CₖₛTₖ | | |
| 936071 | 4903 | 4918 | GCCGTCATAAT | Gₖₛ^{m}Cₖₛ^{m}C_{ds}G_{ds}T_{ds}^{m}C_{ds}A_{ds}T_{ds} | 51 | 1353 |
| | | | CCTGG | A_{ds}A_{ds}T_{ds}^{m}Cₑₛ^{m}CₑₛTₑₛGₖₛGₖ | | |
| 936072 | 4906 | 4921 | TGCGCCGTCAT | TₖₛGₖₛ^{m}C_{ds}G_{ds}^{m}C_{ds}^{m}C_{ds}G_{ds}T_{ds} | 77 | 1581 |
| | | | AATCC | ^{m}C_{ds}A_{ds}T_{ds}AₑₛAₑₛTₑₛ^{m}Cₖₛ^{m}Cₖ | | |
| 936073 | 4908 | 4923 | GGTGCGCCGTC | GₖₛGₖₛT_{ds}G_{ds}^{m}C_{ds}G_{ds}^{m}C_{ds}^{m}C_{ds} | 53 | 1658 |
| | | | ATAAT | G_{ds}T_{ds}^{m}C_{ds}AₑₛTₑₛAₑₛAₖₛTₖ | | |
| 936074 | 4910 | 4925 | GTGGTGCGCCG | GₖₛTₖₛG_{ds}G_{ds}T_{ds}G_{ds}^{m}C_{ds}G_{ds}^{m} | 53 | 593 |
| | | | TCATA | C_{ds}^{m}C_{ds}G_{ds}Tₑₛ^{m}CₑₛAₑₛTₖₛAₖ | | |
| 936075 | 5053 | 5068 | CAACTTGCTAC | ^{m}CₖₛAₖₛA_{ds}^{m}C_{ds}T_{ds}T_{ds}G_{ds}^{m}C_{ds} | 57 | 1109 |
| | | | CCCAG | T_{ds}A_{ds}^{m}C_{ds}^{m}Cₑₛ^{m}Cₑₛ^{m}CₑₛAₖₛGₖ | | |
| 936076 | 5772 | 5787 | CCCCGCATACG | ^{m}Cₖₛ^{m}Cₖₛ^{m}C_{ds}^{m}C_{ds}G_{ds}^{m}C_{ds}A_{ds}T_{ds} | 70 | 1707 |
| | | | CCGTA | A_{ds}^{m}C_{ds}G_{ds}^{m}Cₑₛ^{m}CₑₛGₑsTₖₛAₖ | | |
| 936079 | 6361 | 6376 | AGCAAAGGCAT | AₖₛGₖₛ^{m}C_{ds}A_{ds}A_{ds}A_{ds}G_{ds}G_{ds}^{m} | 47 | 678 |
| | | | ACTCC | C_{ds}A_{ds}T_{ds}Aₑₛ^{m}CₑₛTₑₛ^{m}Cₖₛ^{m}Cₖ | | |
| 936080 | 7439 | 7454 | CAGCATGAGTA | ^{m}CₖₛAₖₛG_{ds}^{m}C_{ds}A_{ds}T_{ds}G_{ds}A_{ds} | 59 | 1517 |
| | | | GACGA | G_{ds}T_{ds}A_{ds}GₑₛAₑₛ^{m}CₑₛGₖₛAₖ | | |
| 936081 | 8810 | 8825 | GGATTAAGGCT | GₖₛGₖₛA_{ds}T_{ds}T_{ds}A_{ds}A_{ds}G_{ds}G_{ds}^{m} | 27 | 683 |
| | | | CAGCG | C_{ds}T_{ds}^{m}CₑₛAₑₛGₑₛ^{m}CₖₛGₖ | | |
| 936082 | 8811 | 8826 | TGGATTAAGGC | TₖₛGₖₛG_{ds}A_{ds}T_{ds}T_{ds}A_{ds}A_{ds}G_{ds} | 30 | 1715 |
| | | | TCAGC | G_{ds}^{m}C_{ds}Tₑₛ^{m}CₑₛAₑₛGₖₛ^{m}Cₖ | | |
| 936083 | 9377 | 9392 | GCTTATTAGCA | Gₖₛ^{m}CₖₛT_{ds}T_{ds}A_{ds}T_{ds}T_{ds}A_{ds}G_{ds} | 43 | 1444 |
| | | | GCAGG | ^{m}C_{ds}A_{ds}Gₑₛ^{m}CₑₛAₑₛGₖₛGₖ | | |
| 936084 | 9801 | 9816 | GCTTTATAGAT | Gₖₛ^{m}CₖₛT_{ds}T_{ds}T_{ds}A_{ds}T_{ds}A_{ds}G_{ds} | 40 | 761 |
| | | | GCGGA | A_{ds}T_{ds}Gₑₛ^{m}CₑₛGₑₛGₖₛAₖ | | |
| 936085 | 10157 | 10172 | CCCCACCAAGC | ^{m}Cₖₛ^{m}Cₖₛ^{m}C_{ds}^{m}C_{ds}A_{ds}^{m}C_{ds}^{m}C_{ds} | 74 | 383 |
| | | | CTCGG | A_{ds}A_{ds}G_{ds}^{m}C_{ds}^{m}CₑₛTₑₛ^{m}CₑₛGₖₛ | | |
| | | | | Gₖ | | |
| 936086 | 10172 | 10187 | GCCAAGGAATC | Gₖₛ^{m}Cₖₛ^{m}C_{ds}A_{ds}A_{ds}G_{ds}G_{ds}A_{ds} | 52 | 610 |
| | | | TACTC | A_{ds}T_{ds}^{m}C_{ds}TₑₛAₑₛ^{m}CₑₛTₖₛ^{m}Cₖ | | |
| 936087 | 10272 | 10287 | AGAGAGGTAA | AₖₛGₖₛA_{ds}G_{ds}A_{ds}G_{ds}G_{ds}T_{ds}A_{ds} | 48 | 990 |
| | | | ATCCCC | A_{ds}A_{ds}Tₑₛ^{m}Cₑₛ^{m}Cₑₛ^{m}Cₖₛ^{m}Cₖ | | |
| 936088 | 11641 | 11656 | GACATTTATGG | GₖₛAₖₛ^{m}C_{ds}A_{ds}T_{ds}T_{ds}T_{ds}A_{ds}T_{ds} | 36 | 1893 |
| | | | TGCCC | G_{ds}G_{ds}TₑₛGₑₛ^{m}Cₑₛ^{m}Cₖₛ^{m}Cₖ | | |
| 936108 | 3530 | 3545 | AATAAGCAAGT | AₖₛAₖₛT_{ds}A_{ds}A_{ds}G_{ds}^{m}C_{ds}A_{ds}A_{ds} | 47 | 2242 |
| | | | CTGGT | G_{ds}T_{ds}^{m}CₑₛTₑₛGₑₛGₖₛTₖ | | |
| 936109 | 3684 | 3699 | GTTTTTGCACTT | GₖₛTₖₛT_{ds}T_{ds}T_{ds}T_{ds}G_{ds}^{m}C_{ds}A_{ds} | 54 | 1777 |
| | | | CCTG | ^{m}C_{ds}T_{ds}Tₑₛ^{m}Cₑₛ^{m}CₑₛTₖₛGₖ | | |
| 936110 | 3794 | 3809 | GGTTATACTCA | GₖₛGₖₛT_{ds}T_{ds}A_{ds}T_{ds}A_{ds}^{m}C_{ds}T_{ds} | 25 | 2182 |
| | | | GGCTG | ^{m}C_{ds}A_{ds}GₑₛGₑₛ^{m}CₑₛTₖₛGₖ | | |
| 936111 | 4902 | 4917 | CCGTCATAATC | ^{m}Cₖₛ^{m}CₖₛG_{ds}T_{ds}^{m}C_{ds}A_{ds}T_{ds}A_{ds} | 35 | 1277 |
| | | | CTGGG | A_{ds}T_{ds}^{m}C_{ds}^{m}CₑₛTₑₛGₑₛGₖₛGₖ | | |
| 936112 | 4905 | 4920 | GCGCCGTCATA | Gₖₛ^{m}CₖₛG_{ds}^{m}C_{ds}^{m}C_{ds}G_{ds}T_{ds}^{m}C_{ds} | 46 | 1505 |
| | | | ATCCT | A_{ds}T_{ds}A_{ds}AₑₛTₑₛ^{m}Cₑₛ^{m}CₖₛTₖ | | |
| 936113 | 4907 | 4922 | GTGCGCCGTCA | GₖₛTₖₛG_{ds}^{m}C_{ds}G_{ds}^{m}C_{ds}^{m}C_{ds}G_{ds} | 80 | 2243 |
| | | | TAATC | T_{ds}^{m}C_{ds}A_{ds}TₑₛAₑₛAₑₛTₖₛ^{m}Cₖ | | |
| 936114 | 4909 | 4924 | TGGTGCGCCGT | TₖₛGₖₛG_{ds}T_{ds}G_{ds}^{m}C_{ds}G_{ds}^{m}C_{ds}^{m} | 51 | 517 |
| | | | CATAA | C_{ds}G_{ds}T_{ds}^{m}CₑₛAₑₛTₑₛAₖₛAₖ | | |
| 936115 | 5052 | 5067 | AACTTGCTACC | AₖₛAₖₛ^{m}C_{ds}T_{ds}T_{ds}G_{ds}^{m}C_{ds}T_{ds} | 43 | 1033 |
| | | | CCAGG | A_{ds}^{m}C_{ds}^{m}C_{ds}^{m}Cₑₛ^{m}CₑₛAₑₛGₖₛGₖ | | |
| 936116 | 5771 | 5786 | CCCGCATACGC | ^{m}Cₖₛ^{m}Cₖₛ^{m}C_{ds}G_{ds}^{m}C_{ds}A_{ds}T_{ds}A_{ds} | 59 | 1513 |
| | | | CGTAC | ^{m}C_{ds}G_{ds}^{m}C_{ds}^{m}CₑₛGₑₛTₑₛAₖₛ^{m}Cₖ | | |
| 936117 | 5773 | 5788 | GCCCCGCATAC | Gₖₛ^{m}Cₖₛ^{m}C_{ds}^{m}C_{ds}^{m}C_{ds}G_{ds}^{m}C_{ds} | 47 | 450 |
| | | | GCCGT | A_{ds}T_{ds}A_{ds}^{m}C_{ds}Gₑₛ^{m}Cₑₛ^{m}CₑₛGₖₛ | | |
| | | | | Tₖ | | |
| 936119 | 6360 | 6375 | GCAAAGGCATA | Gₖₛ^{m}CₖₛA_{ds}A_{ds}A_{ds}G_{ds}G_{ds}^{m}C_{ds} | 41 | 2244 |
| | | | CTCCA | A_{ds}T_{ds}A_{ds}^{m}CₑₛTₑₛ^{m}Cₑₛ^{m}CₖₛAₖ | | |
| 936120 | 7438 | 7453 | AGCATGAGTAG | AₖₛGₖₛ^{m}C_{ds}A_{ds}T_{ds}G_{ds}A_{ds}G_{ds}T_{ds} | 68 | 1866 |
| | | | ACGAG | A_{ds}G_{ds}Aₑₛ^{m}CₑₛGₑₛAₖₛGₖ | | |
| 936121 | 8809 | 8824 | GATTAAGGCTC | GₖₛAₖₛT_{ds}T_{ds}A_{ds}A_{ds}G_{ds}G_{ds}^{m}C_{ds} | 39 | 2245 |
| | | | AGCGT | T_{ds}^{m}C_{ds}AₑₛGₑₛ^{m}CₑₛGₖₛTₖ | | |
| 936122 | 9376 | 9391 | CTTATTAGCAG | ^{m}CₖₛTₖₛT_{ds}A_{ds}T_{ds}T_{ds}A_{ds}G_{ds}^{m}C_{ds} | 46 | 2221 |
| | | | CAGGG | A_{ds}G_{ds}^{m}CₑₛAₑₛGₑₛGₖₛGₖ | | |
| 936123 | 9800 | 9815 | CTTTATAGATG | ^{m}CₖₛTₖₛT_{ds}T_{ds}A_{ds}T_{ds}A_{ds}G_{ds}A_{ds} | 45 | 2229 |
| | | | CGGAC | T_{ds}G_{ds}^{m}CₑₛGₑₛGₑₛAₖₛ^{m}Cₖ | | |
| 936124 | 10156 | 10171 | CCCACCAAGCC | ^{m}Cₖₛ^{m}Cₖₛ^{m}C_{ds}A_{ds}^{m}C_{ds}^{m}C_{ds}Adₛ | 47 | 2246 |
| | | | TCGGT | A_{ds}G_{ds}^{m}C_{ds}^{m}C_{ds}Tₑₛ^{m}CₑₛGₑₛGₖₛ | | |
| | | | | Tₖ | | |
| 936125 | 10171 | 10186 | CCAAGGAATCT | ^{m}Cₖₛ^{m}CₖₛA_{ds}A_{ds}G_{ds}G_{ds}A_{ds}A_{ds} | 59 | 1734 |
| | | | ACTCC | T_{ds}^{m}C_{ds}T_{ds}Aₑₛ^{m}CₑₛTₑₛ^{m}Cₖₛ^{m}Cₖ | | |
| 936126 | 10271 | 10286 | GAGAGGTAAAT | GₖₛAₖₛG_{ds}A_{ds}G_{ds}G_{ds}T_{ds}A_{ds}A_{ds} | 55 | 2233 |
| | | | CCCCA | A_{ds}T_{ds}^{m}Cₑₛ^{m}Cₑₛ^{m}Cₑₛ^{m}CₖₛAₖ | | |
| 936127 | 11640 | 11655 | ACATTTATGGT | Aₖₛ^{m}CₖₛA_{ds}T_{ds}T_{ds}T_{ds}A_{ds}T_{ds} | 43 | 992 |
| | | | GCCCT | G_{ds}G_{ds}T_{ds}Gₑₛ^{m}Cₑₛ^{m}Cₑₛ^{m}CₖₛTₖ | | |
| 936146 | 3532 | 3547 | GCAATAAGCAA | Gₖₛ^{m}CₖₛA_{ds}A_{ds}T_{ds}A_{ds}A_{ds}G_{ds}^{m} | 42 | 2247 |
| | | | GTCTG | C_{ds}A_{ds}A_{ds}GₑₛTₑₛ^{m}CₑₛTₖₛGₖ | | |
| 936147 | 3686 | 3701 | CGGTTTTTGCA | ^{m}CₖₛGₖₛG_{ds}T_{ds}T_{ds}T_{ds}T_{ds}T_{ds}G_{ds} | 27 | 1928 |
| | | | CTTCC | ^{m}C_{ds}A_{ds}^{m}CₑₛTₑₛTₑₛ^{m}Cₖₛ^{m}Cₖ | | |
| 936148 | 3796 | 3811 | CGGGTTATACT | ^{m}CₖₛGₖₛG_{ds}G_{ds}T_{ds}T_{ds}A_{ds}T_{ds}A_{ds} | 10 | 2183 |
| | | | CAGGC | ^{m}C_{ds}T_{ds}^{m}CₑₛAₑₛGₑₛGₖₛ^{m}Cₖ | | |
| 936149 | 4904 | 4919 | CGCCGTCATAA | ^{m}CₖₛGₖₛ^{m}C_{ds}^{m}C_{ds}G_{ds}T_{ds}^{m}C_{ds}A_{ds} | 71 | 1429 |
| | | | TCCTG | T_{ds}A_{ds}A_{ds}Tₑₛ^{m}Cₑₛ^{m}CₑₛTₖₛGₖ | | |
| 936150 | 4911 | 4926 | GGTGGTGCGCC | GₖₛGₖₛT_{ds}G_{ds}G_{ds}T_{ds}G_{ds}^{m}C_{ds}G_{ds} | 40 | 669 |
| | | | GTCAT | ^{m}C_{ds}^{m}C_{ds}GₑₛTₑₛ^{m}CₑₛAₖₛTk | | |
| 936151 | 5054 | 5069 | GCAACTTGCTA | Gₖₛ^{m}CₖₛA_{ds}A_{ds}^{m}C_{ds}T_{ds}T_{ds}G_{ds}^{m} | 42 | 1186 |
| | | | CCCCA | C_{ds}T_{ds}A_{ds}^{m}Cₑₛ^{m}Cₑₛ^{m}Cₑₛ^{m}CₖₛAₖ | | |
| 936154 | 6362 | 6377 | AAGCAAAGGC | AₖₛAₖₛG_{ds}^{m}C_{ds}A_{ds}A_{ds}A_{ds}G_{ds}G_{ds} | 44 | 2248 |
| | | | ATACTC | ^{m}C_{ds}A_{ds}TₑₛAₑₛ^{m}CₑₛTₖₛ^{m}Cₖ | | |
| 936155 | 7440 | 7455 | TCAGCATGAGT | Tₖₛ^{m}CₖₛA_{ds}G_{ds}^{m}C_{ds}A_{ds}T_{ds}G_{ds}A_{ds} | 63 | 1942 |
| | | | AGACG | G_{ds}T_{ds}AₑₛGₑₛAₑₛ^{m}CₖₛGₖ | | |
| 936156 | 8812 | 8827 | CTGGATTAAGG | ^{m}CₖₛTₖₛG_{ds}G_{ds}A_{ds}T_{ds}T_{ds}A_{ds}A_{ds} | 53 | 759 |
| | | | CTCAG | G_{ds}G_{ds}^{m}CₑₛTₑₛ^{m}CₑₛAₖₛGₖ | | |
| 936157 | 9378 | 9393 | TGCTTATTAGC | TₖₛGₖₛ^{m}C_{ds}T_{ds}T_{ds}A_{ds}T_{ds}T_{ds}A_{ds} | 59 | 2222 |
| | | | AGCAG | G_{ds}^{m}C_{ds}AₑₛGₑₛ^{m}CₑₛAₖₛGₖ | | |
| 936158 | 9802 | 9817 | TGCTTTATAGA | TₖₛGₖₛ^{m}C_{ds}T_{ds}T_{ds}T_{ds}A_{ds}T_{ds}A_{ds} | 28 | 2230 |
| | | | TGCGG | G_{ds}A_{ds}TₑₛGₑₛ^{m}CₑₛGₖₛGₖ | | |
| 936159 | 10158 | 10173 | TCCCCACCAAG | Tₖₛ^{m}Cₖₛ^{m}C_{ds}^{m}C_{ds}^{m}C_{ds}A_{ds}^{m}C_{ds}^{m} | 63 | 2249 |
| | | | CCTCG | C_{ds}A_{ds}A_{ds}G_{ds}^{m}Cₑₛ^{m}CₑₛTₑₛ^{m}Cₖₛ | | |
| | | | | Gₖ | | |
| 936160 | 10173 | 10188 | TGCCAAGGAAT | TₖₛGₖₛ^{m}C_{ds}^{m}C_{ds}A_{ds}A_{ds}G_{ds}G_{ds}A_{ds} | 50 | 1810 |
| | | | CTACT | A_{ds}T_{ds}^{m}CₑₛTₑₛAₑₛ^{m}CₖₛTₖ | | |
| 936161 | 10273 | 10288 | CAGAGAGGTAA | ^{m}CₖₛAₖₛG_{ds}A_{ds}G_{ds}A_{ds}G_{ds}G_{ds}T_{ds} | 65 | 2234 |
| | | | ATCCC | A_{ds}A_{ds}AₑₛTₑₛ^{m}Cₑₛ^{m}Cₖₛ^{m}Cₖ | | |
| 936431 | 14113 | 14128 | GTGTGTAGAAG | GₖₛTₖₛGₖₛT_{ds}G_{ds}T_{ds}A_{ds}G_{ds}A_{ds} | 46 | 2250 |
| | | | TGCCA | A_{ds}G_{ds}T_{ds}G_{ds}^{m}Cₖₛ^{m}CₖₛAₖ | | |
| 936432 | 14114 | 14129 | TGTGTGTAGAA | TₖₛGₖₛTₖₛG_{ds}T_{ds}G_{ds}T_{ds}A_{ds}G_{ds} | 61 | 2251 |
| | | | GTGCC | A_{ds}A_{ds}G_{ds}T_{ds}Gₖₛ^{m}Cₖₛ^{m}Cₖ | | |
| 936434 | 14116 | 14131 | ACTGTGTGTAG | Aₖₛ^{m}CₖₛTₖₛG_{ds}T_{ds}G_{ds}T_{ds}G_{ds}T_{ds} | 113 | 2252 |
| | | | AAGTG | A_{ds}G_{ds}A_{ds}A_{ds}GₖₛTₖₛGₖ | | |
| 936435 | 14117 | 14132 | GACTGTGTGTA | GₖₛAₖₛ^{m}CₖₛT_{ds}G_{ds}T_{ds}G_{ds}T_{ds}G_{ds} | 81 | 2253 |
| | | | GAAGT | T_{ds}A_{ds}G_{ds}A_{ds}AₖₛGₖₛTₖ | | |
| 936441 | 14209 | 14224 | ATATCATCCAG | AₖₛTₖₛAₖₛT_{ds}^{m}C_{ds}A_{ds}T_{ds}^{m}C_{ds}^{m} | 81 | 2254 |
| | | | CACCT | C_{ds}A_{ds}G_{ds}^{m}C_{ds}A_{ds}^{m}Cₖₛ^{m}CₖₛTₖ | | |
| 936442 | 14214 | 14229 | AATTCATATCA | AₖₛAₖₛTₖₛT_{ds}^{m}C_{ds}A_{ds}T_{ds}A_{ds}T_{ds} | 36 | 2255 |
| | | | TCCAG | ^{m}C_{ds}A_{ds}T_{ds}^{m}C_{ds}^{m}CₖₛAₖₛGₖ | | |
| 936444 | 14221 | 14236 | GAGCTTGAATT | GₖₛAₖₛGₖₛ^{m}C_{ds}T_{ds}T_{ds}G_{ds}A_{ds}A_{ds} | 73 | 2256 |
| | | | CATAT | T_{ds}T_{ds}^{m}C_{ds}A_{ds}TₖₛAₖₛTₖ | | |
| 936446 | 14675 | 14690 | GATGGATTGAT | GₖₛAₖₛTₖₛG_{ds}G_{ds}A_{ds}T_{ds}T_{ds}G_{ds} | 55 | 2257 |
| | | | GAGCA | A_{ds}T_{ds}G_{ds}A_{ds}Gₖₛ^{m}CₖₛAₖ | | |
| 936447 | 14676 | 14691 | GGATGGATTGA | GₖₛGₖₛAₖₛT_{ds}G_{ds}G_{ds}A_{ds}T_{ds}T_{ds} | 57 | 2258 |
| | | | TGAGC | G_{ds}A_{ds}T_{ds}G_{ds}AₖₛGₖₛ^{m}Cₖ | | |
| 936449 | 15382 | 15397 | TTCGGCCCAGA | TₖₛTₖₛmCₖₛG_{ds}G_{ds}mC_{ds}mC_{ds}mC_{ds} | 73 | 2259 |
| | | | GAGGT | A_{ds}G_{ds}A_{ds}G_{ds}A_{ds}GₖₛGₖₛTₖ | | |
| 936450 | 15383 | 15398 | TTTCGGCCCAG | TₖₛTₖₛTₖₛ^{m}C_{ds}G_{ds}G_{ds}^{m}C_{ds}^{m}C_{ds}^{m} | 77 | 2260 |
| | | | AGAGG | C_{ds}A_{ds}G_{ds}A_{ds}G_{ds}AₖₛGₖₛGₖ | | |
| 936451 | 15384 | 15399 | TTTTCGGCCCA | TₖₛTₖₛTₖₛT_{ds}^{m}C_{ds}G_{ds}G_{ds}^{m}C_{ds}^{m} | 78 | 2261 |
| | | | GAGAG | C_{ds}^{m}C_{ds}A_{ds}G_{ds}A_{ds}GₖₛAₖₛGₖ | | |
| 936452 | 15385 | 15400 | CTTTTCGGCCC | ^{m}CₖₛTₖₛTₖₛT_{ds}T_{ds}^{m}C_{ds}G_{ds}G_{ds}^{m} | 51 | 2262 |
| | | | AGAGA | C_{ds}^{m}C_{ds}^{m}C_{ds}A_{ds}G_{ds}AₖₛGₖₛAₖ | | |
| 936453 | 15386 | 15401 | GCTTTTCGGCC | Gₖₛ^{m}CₖₛTₖₛT_{ds}T_{ds}T_{ds}mC_{ds}G_{ds} | 36 | 2263 |
| | | | CAGAG | G_{ds}^{m}C_{ds}^{m}C_{ds}^{m}C_{ds}A_{ds}GₖₛAₖₛGₖ | | |
| 936454 | 15388 | 15403 | CTGCTTTTCGG | ^{m}CₖₛTₖₛGₖₛ^{m}C_{ds}T_{ds}T_{ds}T_{ds}T_{ds}^{m} | 41 | 2264 |
| | | | CCCAG | C_{ds}G_{ds}G_{ds}^{m}C_{ds}^{m}C_{ds}^{m}CₖₛAₖₛGₖ | | |
| 936455 | 15389 | 15404 | ACTGCTTTTCG | AₖₛmCₖₛTₖₛG_{ds}^{m}C_{ds}T_{ds}T_{ds}T_{ds}T_{ds} | 43 | 2265 |
| | | | GCCCA | ^{m}C_{ds}G_{ds}G_{ds}^{m}C_{ds}^{m}Cₖₛ^{m}CₖₛAₖ | | |
| 936456 | 15390 | 15405 | AACTGCTTTTC | AₖₛAₖₛ^{m}CₖₛT_{ds}G_{ds}^{m}C_{ds}T_{ds}T_{ds} | 36 | 2266 |
| | | | GGCCC | T_{ds}T_{ds}^{m}C_{ds}G_{ds}G_{ds}^{m}Cₖₛ^{m}Cₖₛ^{m}Cₖ | | |
| 936457 | 15391 | 15406 | GAACTGCTTTT | GₖₛAₖₛAₖₛ^{m}C_{ds}T_{ds}G_{ds}^{m}C_{ds}T_{ds} | 46 | 2267 |
| | | | CGGCC | T_{ds}T_{ds}T_{ds}^{m}C_{ds}G_{ds}Gₖₛ^{m}Cₖₛ^{m}Cₖ | | |
| 936458 | 15392 | 15407 | GGAACTGCTTT | GₖₛGₖₛAₖₛA_{ds}^{m}C_{ds}T_{ds}G_{ds}^{m}C_{ds} | 28 | 2268 |
| | | | TCGGC | T_{ds}T_{ds}T_{ds}T_{ds}^{m}C_{ds}GₖₛGₖₛ^{m}Cₖ | | |
| 936480 | 17621 | 17636 | TACTGTGGTGT | TₖₛAₖₛ^{m}CₖₛT_{ds}G_{ds}T_{ds}G_{ds}G_{ds}T_{ds} | 72 | 2269 |
| | | | GCAGA | G_{ds}T_{ds}G_{ds}^{m}C_{ds}AₖₛGₖₛAₖ | | |
| 936481 | 17622 | 17637 | ATACTGTGGTG | AₖₛTₖₛAₖₛ^{m}C_{ds}T_{ds}G_{ds}T_{ds}G_{ds}G_{ds} | 68 | 2270 |
| | | | TGCAG | T_{ds}G_{ds}T_{ds}G_{ds}^{m}CₖₛAₖₛGₖ | | |
| 936482 | 17623 | 17638 | CATACTGTGGT | ^{m}CₖₛAₖₛTₖₛA_{ds}^{m}C_{ds}T_{ds}G_{ds}T_{ds}G_{ds} | 43 | 2271 |
| | | | GTGCA | G_{ds}T_{ds}G_{ds}T_{ds}Gₖₛ^{m}CₖₛAₖ | | |
| 936485 | 17627 | 17642 | AAGACATACTG | AₖₛAₖₛGₖₛA_{ds}^{m}C_{ds}A_{ds}T_{ds}A_{ds}^{m} | 45 | 2272 |
| | | | TGGTG | C_{ds}T_{ds}G_{ds}T_{ds}G_{ds}GₖₛTₖₛGₖ | | |
| 936488 | 17637 | 17652 | GGCAATACCCA | GₖₛGₖₛ^{m}CₖₛA_{ds}A_{ds}T_{ds}A_{ds}^{m}C_{ds}^{m} | 45 | 2273 |
| | | | AGACA | C_{ds}^{m}C_{ds}A_{ds}A_{ds}G_{ds}Aₖₛ^{m}CₖₛAₖ | | |

TABLE 32
Reduction of SPDEF RNA by 4 μM modified oligonucleotides

| | SEQ | SEQ | SEQ | SEQ | | | | |
|---|---|---|---|---|---|---|---|---|
| | ID | ID | ID | ID | | | | |
| | NO: 1 | NO: 1 | NO: 2 | NO: 2 | | | | SEQ |
| Compound | Start | Stop | Start | Stop | Sequence | Chemistry Notation | (% | ID |
| Number | Site | Site | Site | Site | (5′ to 3′) | (5′ to 3′) | UTC) | NO |
| 833814 | N/A | N/A | 12009 | 12024 | ACCAACA | Aₖₛ^{m}Cₖₛ^{m}CₖₛA_{ds}A_{ds}^{m}C_{ds}A_{ds}G_{ds} | 42 | 993 |
| | | | | | GATATAC | A_{ds}T_{ds}A_{ds}T_{ds}A_{ds}^{m}CₖₛGₖₛ^{m}Cₖ | | |
| | | | | | GC | | | |
| 854302 | N/A | N/A | 8811 | 8826 | TGGATTA | TₖₛGₖₛGₖₛA_{ds}T_{ds}T_{ds}A_{ds}A_{ds}G_{ds} | 34 | 1715 |
| | | | | | AGGCTCA | G_{ds}^{m}C_{ds}T_{ds}^{m}C_{ds}AₖₛGₖₛ^{m}Cₖ | | |
| | | | | | GC | | | |
| 936089 | N/A | N/A | 12004 | 12019 | CAGATAT | ^{m}CₖₛAₖₛG_{ds}A_{ds}T_{ds}A_{ds}T_{ds}A_{ds}^{m} | | |
| | | | | | ACGCTCCT | C_{ds}G_{ds}^{m}C_{ds}Tₑₛ^{m}Cₑₛ^{m}CₑₛTₖₛAₖ | 40 | 1895 |
| | | | | | A | | | |
| 936090 | N/A | N/A | 12006 | 12021 | AACAGAT | AₖₛAₖₛ^{m}C_{ds}A_{ds}G_{ds}A_{ds}T_{ds}A_{ds}T_{ds} | | |
| | | | | | ATACGCTC | A_{ds}^{m}C_{ds}Gₑₛ^{m}CₑₛTₑₛ^{m}Cₖₛ^{m}Cₖ | 60 | 1971 |
| | | | | | C | | | |
| 936091 | 492 | 507 | 13603 | 13618 | GCGACAC | Gₖₛ^{m}CₖₛG_{ds}A_{ds}^{m}C_{ds}A_{ds}^{m}C_{ds}^{m}C_{ds} | 58 | 255 |
| | | | | | CGTGTCG | G_{ds}T_{ds}G_{ds}Tₑₛ^{m}CₑₛGₑₛGₖₛGₖ | | |
| | | | | | GG | | | |
| 936092 | 498 | 513 | 13609 | 13624 | CTGTCCGC | ^{m}CₖₛTₖₛG_{ds}T_{ds}^{m}C_{ds}^{m}C_{ds}G_{ds}^{m}C_{ds} | 63 | 1089 |
| | | | | | GACACCG | G_{ds}A_{ds}^{m}C_{ds}Aₑₛ^{m}Cₑₛ^{m}CₑₛGₖₛTₖ | | |
| | | | | | T | | | |
| 936093 | 810 | 825 | 13921 | 13936 | GCACTTCG | Gₖₛ^{m}CₖₛA_{ds}^{m}C_{ds}T_{ds}T_{ds}^{m}C_{ds}G_{ds} | 57 | 563 |
| | | | | | CCCACCA | ^{m}C_{ds}^{m}C_{ds}^{m}C_{ds}Aₑₛ^{m}Cₑₛ^{m}CₑₛAₖₛ^{m} | | |
| | | | | | C | Cₖ | | |
| 936094 | 829 | 844 | 13940 | 13955 | GCCGTCTC | Gₖₛ^{m}Cₖₛ^{m}C_{ds}G_{ds}T_{ds}^{m}C_{ds}T_{ds}^{m}C_{ds} | 64 | 1552 |
| | | | | | GATGTCCT | G_{ds}A_{ds}T_{ds}GₑₛTₑₛ^{m}Cₑₛ^{m}CₖₛTₖ | | |
| 936096 | N/A | N/A | 14213 | 14228 | ATTCATAT | AₖₛTₖₛT_{ds}^{m}C_{ds}A_{ds}T_{ds}A_{ds}T_{ds}^{m}C_{ds} | 33 | 1606 |
| | | | | | CATCCAG | A_{ds}T_{ds}MCₑₛ^{m}CₑₛAₑₛGₖₛ^{m}Cₖ | | |
| | | | | | C | | | |
| 936097 | N/A | N/A | 14215 | 14230 | GAATTCAT | GₖₛAₖₛA_{ds}T_{ds}T_{ds}^{m}C_{ds}A_{ds}T_{ds}A_{ds} | 39 | 1682 |
| | | | | | ATCATCCA | T_{ds}^{m}C_{ds}AₑₛTₑₛ^{m}Cₑₛ^{m}CₖₛAₖ | | |
| 936098 | N/A | N/A | 15387 | 15402 | TGCTTTTC | TₖₛGₖₛ^{m}C_{ds}T_{ds}T_{ds}T_{ds}T_{ds}^{m}C_{ds}G_{ds} | 47 | 999 |
| | | | | | GGCCCAG | G_{ds}^{m}C_{ds}^{m}Cₑₛ^{m}CₑₛAₑₛGₖₛAₖ | | |
| | | | | | A | | | |
| 936099 | N/A | N/A | 16598 | 16613 | TGAACTTG | TₖₛGₖₛA_{ds}A_{ds}^{m}C_{ds}T_{ds}T_{ds}G_{ds}G_{ds} | 60 | 1686 |
| | | | | | GTTCAGG | T_{ds}T_{ds}^{m}CₑₛAₑₛGₑₛGₖₛGₖ | | |
| | | | | | G | | | |
| 936100 | N/A | N/A | 17291 | 17306 | ACGGTTGT | Aₖₛ^{m}CₖₛG_{ds}G_{ds}T_{ds}T_{ds}G_{ds}T_{ds}^{m}C_{ds} | 33 | 2056 |
| | | | | | CCCCAGCT | ^{m}C_{ds}^{m}C_{ds}^{m}CₑₛAₑₛGₑₛ^{m}CₖₛTₖ | | |
| 936101 | N/A | N/A | 17292 | 17307 | CACGGTT | ^{m}CₖₛAₖₛ^{m}C_{ds}G_{ds}G_{ds}T_{ds}T_{ds}G_{ds}T_{ds} | 54 | 163 |
| | | | | | GTCCCCA | ^{m}C_{ds}^{m}C_{ds}^{m}Cₑₛ^{m}CₑₛAₑₛGₖₛ^{m}Cₖ | | |
| | | | | | GC | | | |
| 936102 | N/A | N/A | 17303 | 17318 | TTCCTAGT | TₖₛTₖₛ^{m}C_{ds}^{m}C_{ds}T_{ds}A_{ds}G_{ds}T_{ds}A_{ds} | 32 | 1754 |
| | | | | | ATCCACG | T_{ds}^{m}C_{ds}^{m}CₑₛAₑₛ^{m}CₑₛGₖₛGₖ | | |
| | | | | | G | | | |
| 936103 | N/A | N/A | 17305 | 17320 | ACTTCCTA | Aₖₛ^{m}CₖₛT_{ds}T_{ds}^{m}C_{ds}^{m}C_{ds}T_{ds}A_{ds} | 57 | 1906 |
| | | | | | GTATCCAC | G_{ds}T_{ds}A_{ds}Tₑₛ^{m}Cₑₛ^{m}CₑₛAₖₛ^{m}Cₖ | | |
| 936104 | N/A | N/A | 17493 | 17508 | AACTTGTA | AₖₛAₖₛ^{m}C_{ds}T_{ds}T_{ds}G_{ds}T_{ds}A_{ds}A_{ds} | 26 | 2059 |
| | | | | | ACAGTGG | ^{m}C_{ds}A_{ds}GₑₛTₑₛGₑₛGₖₛTₖ | | |
| | | | | | T | | | |
| 936105 | N/A | N/A | 17525 | 17540 | TTCATAGA | TₖₛTₖₛ^{m}C_{ds}A_{ds}T_{ds}A_{ds}G_{ds}A_{ds}^{m}C_{ds} | 71 | 240 |
| | | | | | CTTTCCCT | T_{ds}T_{ds}Tₑₛ^{m}Cₑₛ^{m}Cₑₛ^{m}CₖₛTₖ | | |
| 936106 | 1747 | 1762 | 20032 | 20047 | TGTCGAGT | TₖₛGₖₛT_{ds}^{m}C_{ds}G_{ds}A_{ds}G_{ds}T_{ds}^{m}C_{ds} | 37 | 727 |
| | | | | | CACTGCCC | A_{ds}^{m}C_{ds}TₑₛGₑₛ^{m}Cₑₛ^{m}Cₖₛ^{m}Cₖ | | |
| 936107 | 1894 | 1909 | 20179 | 20194 | TCTCTAGT | Tₖₛ^{m}CₖₛT_{ds}^{m}C_{ds}T_{ds}A_{ds}G_{ds}T_{ds}A_{ds} | 65 | 364 |
| | | | | | ATCTTTAT | T_{ds}^{m}C_{ds}TₑₛTₑₛTₑₛAₖₛTₖ | | |
| 936128 | N/A | N/A | 12003 | 12018 | AGATATA | AₖₛGₖₛA_{ds}T_{ds}A_{ds}T_{ds}A_{ds}^{m}C_{ds}G_{ds} | 41 | 1819 |
| | | | | | CGCTCCTA | ^{m}C_{ds}T_{ds}^{m}Cₑₛ^{m}CₑₛTₑₛAₖₛAₖ | | |
| | | | | | A | | | |
| 936129 | N/A | N/A | 12005 | 12020 | ACAGATA | AₖₛGₖₛA_{ds}T_{ds}A_{ds}T_{ds}A_{ds}^{m}C_{ds}G_{ds} | | |
| | | | | | TACGCTCC | ^{m}C_{ds}G_{ds}^{m}CₑₛTₑₛ^{m}Cₑₛ^{m}CₖₛTₖ | 55 | 917 |
| | | | | | T | | | |
| 936130 | 491 | 506 | 13602 | 13617 | CGACACC | ^{m}CₖₛGₖₛA_{ds}^{m}C_{ds}A_{ds}^{m}C_{ds}^{m}C_{ds}G_{ds} | 70 | 2274 |
| | | | | | GTGTCGG | T_{ds}G_{ds}T_{ds}^{m}CₑₛGₑₛGₑₛGₖₛGₖ | | |
| | | | | | GG | | | |
| 936131 | 497 | 512 | 13608 | 13623 | TGTCCGCG | TₖₛGₖₛT_{ds}^{m}C_{ds}^{m}C_{ds}G_{ds}^{m}C_{ds}G_{ds} | 74 | 1014 |
| | | | | | ACACCGT | A_{ds}^{m}C_{ds}A_{ds}^{m}Cₑₛ^{m}CₑₛGₑₛTₖₛGₖ | | |
| | | | | | G | | | |
| 936132 | 809 | 824 | 13920 | 13935 | CACTTCGC | ^{m}CₖₛAₖₛ^{m}C_{ds}T_{ds}T_{ds}^{m}C_{ds}G_{ds}^{m}C_{ds} | 59 | 487 |
| | | | | | CCACCAC | ^{m}C_{ds}^{m}C_{ds}A_{ds}^{m}Cₑₛ^{m}CₑₛAₑₛ^{m}Cₖₛ | | |
| | | | | | C | ^{m}Cₖ | | |
| 936133 | 828 | 843 | 13939 | 13954 | CCGTCTCG | ^{m}Cₖₛ^{m}CₖₛG_{ds}T_{ds}^{m}C_{ds}T_{ds}^{m}C_{ds}G_{ds} | 57 | 37 |
| | | | | | ATGTCCTT | A_{ds}T_{ds}G_{ds}Tₑₛ^{m}Cₑₛ^{m}CₑₛTₖₛTₖ | | |
| 936135 | N/A | N/A | 14212 | 14227 | TTCATATC | TₖₛTₖₛ^{m}C_{ds}A_{ds}T_{ds}A_{ds}T_{ds}^{m}C_{ds}G_{ds} | 24 | 2275 |
| | | | | | ATCCAGC | T_{ds}^{m}C_{ds}^{m}CₑₛAₑₛGₑₛ^{m}CₖₛAₖ | | |
| | | | | | A | | | |
| 936136 | N/A | N/A | 14214 | 14229 | AATTCATA | AₖₛAₖₛT_{ds}T_{ds}^{m}C_{ds}A_{ds}T_{ds}A_{ds}T_{ds} | 53 | 2255 |
| | | | | | TCATCCAG | ^{m}C_{ds}A_{ds}Tₑₛ^{m}Cₑₛ^{m}CₑₛAₖₛGₖ | | |
| 936137 | N/A | N/A | 15386 | 15401 | GCTTTTCG | Gₖₛ^{m}CₖₛT_{ds}T_{ds}T_{ds}T_{ds}^{m}C_{ds}G_{ds}G_{ds} | 31 | 2263 |
| | | | | | GCCCAGA | ^{m}C_{ds}^{m}C_{ds}^{m}CₑₛAₑₛGₑₛAₖₛGₖ | | |
| | | | | | G | | | |
| 936138 | N/A | N/A | 16597 | 16612 | GAACTTG | GₖₛAₖₛA_{ds}^{m}C_{ds}T_{ds}T_{ds}G_{ds}G_{ds} | 46 | 2276 |
| | | | | | GTTCAGG | T_{ds}T_{ds}^{m}C_{ds}AₑₛGₑₛGₑₛGₖₛ^{m}Cₖ | | |
| | | | | | GC | | | |
| 936139 | N/A | N/A | 17290 | 17305 | CGGTTGTC | ^{m}CₖₛGₖₛG_{ds}T_{ds}T_{ds}G_{ds}T_{ds}^{m}C_{ds}^{m} | 32 | 2277 |
| | | | | | CCCAGCTC | C_{ds}^{m}C_{ds}^{m}C_{ds}AₑₛGₑₛ^{m}CₑₛTₖₛ^{m}Cₖ | | |
| 936140 | N/A | N/A | 17302 | 17317 | TCCTAGTA | Tₖₛ^{m}Cₖₛ^{m}C_{ds}T_{ds}A_{ds}G_{ds}T_{ds}A_{ds}T_{ds} | 47 | 1230 |
| | | | | | TCCACGGT | ^{m}C_{ds}^{m}C_{ds}Aₑₛ^{m}CₑₛGₑₛGₖₛTₖ | | |
| 936141 | N/A | N/A | 17304 | 17319 | CTTCCTAG | ^{m}CₖₛTₖₛT_{ds}^{m}C_{ds}^{m}C_{ds}T_{ds}A_{ds}G_{ds} | 38 | 1830 |
| | | | | | TATCCACG | T_{ds}A_{ds}T_{ds}^{m}Cₑₛ^{m}CₑₛAₑₛ^{m}CₖₛGₖ | | |
| 936142 | N/A | N/A | 17492 | 17507 | ACTTGTAA | Aₖₛ^{m}CₖₛT_{ds}T_{ds}G_{ds}T_{ds}A_{ds}A_{ds}^{m}C_{ds} | 21 | 1983 |
| | | | | | CAGTGGTT | A_{ds}G_{ds}TₑₛGₑₛGₑₛTₖₛTₖ | | |
| 936143 | N/A | N/A | 17524 | 17539 | TCATAGA | Tₖₛ^{m}CₖₛA_{ds}T_{ds}A_{ds}G_{ds}Ad^{m}C_{ds}T_{ds} | 47 | 2278 |
| | | | | | CTTTCCCT | T_{ds}T_{ds}^{m}Cₑₛ^{m}Cₑₛ^{m}CₑₛTₖₛGₖ | | |
| | | | | | G | | | |
| 936144 | 1746 | 1761 | 20031 | 20046 | GTCGAGT | GₖₛTₖₛ^{m}C_{ds}G_{ds}A_{ds}G_{ds}T_{ds}^{m}C_{ds} | 43 | 58 |
| | | | | | CACTGCCC | A_{ds}^{m}C_{ds}T_{ds}Gₑₛ^{m}Cₑₛ^{m}Cₑₛ^{m}Cₑₛ^{m}Cₖₛ | | |
| | | | | | T | Tₖ | | |
| 936145 | 1893 | 1908 | 20178 | 20193 | CTCTAGTA | ^{m}CₖₛTₖₛ^{m}C_{ds}T_{ds}A_{ds}G_{ds}T_{ds}A_{ds}T_{ds} | 61 | 2279 |
| | | | | | TCTTTATT | ^{m}C_{ds}T_{ds}TₑₛTₑₛAₑₛTₖₛTₖ | | |
| 936162 | N/A | N/A | 11642 | 11657 | TGACATTT | TₖₛkGₖₛA_{ds}^{m}C_{ds}A_{ds}T_{ds}T_{ds}T_{ds}A_{ds} | 51 | 1068 |
| | | | | | ATGGTGC | T_{ds}G_{ds}GₑₛTₑₛGₑₛ^{m}Cₖₛ^{m}Cₖ | | |
| | | | | | C | | | |
| 936163 | N/A | N/A | 12007 | 12022 | CAACAGA | ^{m}CₖₛAₖₛA_{ds}^{m}C_{ds}A_{ds}G_{ds}T_{ds}T_{ds} | 43 | 2047 |
| | | | | | TATACGCT | A_{ds}T_{ds}A_{ds}^{m}CₑₛGₑₛ^{m}CₑₛTₖₛ^{m}Cₖ | | |
| | | | | | C | | | |
| 936164 | 493 | 508 | 13604 | 13619 | CGCGACA | ^{m}CₖₛGₖₛ^{m}C_{ds}G_{ds}A_{ds}^{m}C_{ds}A_{ds}^{m}C_{ds} | 77 | 861 |
| | | | | | CCGTGTCG | ^{m}C_{ds}G_{ds}T_{ds}GₑₛTₑₛ^{m}CₑₛGₖₛGₖ | | |
| | | | | | G | | | |
| 936165 | 499 | 514 | 13610 | 13625 | CCTGTCCG | ^{m}Cₖₛ^{m}CₖₛT_{ds}G_{ds}T_{ds}^{m}C_{ds}^{m}C_{ds}G_{ds} | 43 | 1165 |
| | | | | | CGACACC | ^{m}C_{ds}G_{ds}A_{ds}^{m}CₑₛAₑₛ^{m}Cₑₛ^{m}CₖₛGₖ | | |
| | | | | | G | | | |
| 936166 | 811 | 826 | 13922 | 13937 | AGCACTTC | AₖₛGₖₛ^{m}C_{ds}A_{ds}^{m}C_{ds}T_{ds}T_{ds}^{m}C_{ds} | 46 | 640 |
| | | | | | GCCCACC | G_{ds}^{m}C_{ds}^{m}C_{ds}^{m}CₑₛAₑₛ^{m}Cₑₛ^{m}Cₖₛ | | |
| | | | | | A | Aₖ | | |
| 936167 | 830 | 845 | 13941 | 13956 | GGCCGTCT | GₖₛGₖₛ^{m}C_{ds}^{m}C_{ds}G_{ds}T_{ds}^{m}C_{ds}T_{ds} | 75 | 114 |
| | | | | | CGATGTCC | ^{m}C_{ds}G_{ds}A_{ds}TₑₛGₑₛTₑₛ^{m}Cₖₛ^{m}Cₖ | | |
| 936169 | N/A | N/A | 14216 | 14231 | TGAATTCA | TₖₛGₖₛA_{ds}A_{ds}T_{ds}T_{ds}^{m}C_{ds}A_{ds}T_{ds} | 37 | 2280 |
| | | | | | TATCATCC | A_{ds}T_{ds}^{m}CₑₛAₑₛTₑₛ^{m}Cₖₛ^{m}Cₖ | | |
| 936170 | N/A | N/A | 15388 | 15403 | CTGCTTTT | ^{m}CₖₛTₖₛG_{ds}^{m}C_{ds}T_{ds}T_{ds}T_{ds}T_{ds}^{m} | 56 | 2264 |
| | | | | | CGGCCCA | C_{ds}G_{ds}G_{ds}^{m}Cₑₛ^{m}Cₑₛ^{m}CₑₛAₖₛGₖ | | |
| | | | | | G | | | |
| 936171 | N/A | N/A | 16599 | 16614 | ATGAACTT | AₖₛTₖₛG_{ds}A_{ds}A_{ds}^{m}C_{ds}T_{ds}T_{ds}G_{ds} | 86 | 2281 |
| | | | | | GGTTCAG | G_{ds}T_{ds}Tₑₛ^{m}CₑₛAₑₛGₖₛGₖ | | |
| | | | | | G | | | |
| 936172 | N/A | N/A | 17293 | 17308 | CCACGGTT | ^{m}Cₖₛ^{m}CₖₛA_{ds}^{m}C_{ds}G_{ds}G_{ds}T_{ds}T_{ds} | 47 | 2132 |
| | | | | | GTCCCCA | G_{ds}T_{ds}^{m}C_{ds}^{m}Cₑₛ^{m}Cₑₛ^{m}CₑₛmAₖₛGₖ | | |
| | | | | | G | | | |
| 936173 | N/A | N/A | 17306 | 17321 | GACTTCCT | GₖₛAₖₛ^{m}C_{ds}T_{ds}Ts^{m}C_{ds}^{m}C_{ds}T_{ds} | 64 | 1982 |
| | | | | | AGTATCC | A_{ds}G_{ds}T_{ds}AₑₛTₑₛ^{m}Cₑₛ^{m}CₖₛAₖ | | |
| | | | | | A | | | |
| 936174 | N/A | N/A | 17494 | 17509 | AAACTTGT | AₖₛAₖₛA_{ds}^{m}C_{ds}T_{ds}T_{ds}G_{ds}T_{ds}A_{ds} | 30 | 2282 |
| | | | | | AACAGTG | A_{ds}^{m}C_{ds}AₑₛGₑₛTₑₛGₖₛGₖ | | |
| | | | | | G | | | |
| 936175 | N/A | N/A | 17526 | 17541 | ATTCATAG | AₖₛTₖₛT_{ds}^{m}C_{ds}A_{ds}T_{ds}A_{ds}G_{ds}A_{ds} | 45 | 2283 |
| | | | | | ACTTTCCC | ^{m}C_{ds}T_{ds}TₑₛTₑₛ^{m}Cₑₛ^{m}Cₖₛ^{m}Cₖ | | |
| 936176 | 1748 | 1763 | 20033 | 20048 | TTGTCGAG | TₖₛTₖₛG_{ds}T_{ds}^{m}C_{ds}G_{ds}A_{ds}G_{ds}T_{ds} | 47 | 803 |
| | | | | | TCACTGCC | ^{m}C_{ds}A_{ds}^{m}CₑₛTₑₛGₑₛ^{m}Cₖₛ^{m}Cₖ | | |
| 936177 | 1895 | 1910 | 20180 | 20195 | TTCTCTAG | TₖₛTₖₛ^{m}C_{ds}T_{ds}^{m}C_{ds}T_{ds}A_{ds}G_{ds}T_{ds} | 64 | 2284 |
| | | | | | TATCTTTA | A_{ds}T_{ds}^{m}CₑₛTₑₛTₑₛTₖₛAₖ | | |
| 936178 | N/A | N/A | 3531 | 3546 | CAATAAG | ^{m}CₖₛAₖₛA_{ds}T_{ds}A_{ds}A_{ds}G_{ds}^{m}C_{ds} | 41 | 1129 |
| | | | | | CAAGTCT | A_{ds}A_{ds}G_{ds}Tₑₛ^{m}CₖₛTₑₛGₖₛGₑ | | |
| | | | | | GG | | | |
| 936179 | N/A | N/A | 3685 | 3700 | GGTTTTTG | GₖₛGₖₛT_{ds}T_{ds}T_{ds}T_{ds}T_{ds}G_{ds}^{m}C_{ds} | 31 | 1852 |
| | | | | | CACTTCCT | A_{ds}^{m}C_{ds}TₑₛTₖₛ^{m}Cₑₛ^{m}CₖₛTₑ | | |
| 936180 | N/A | N/A | 3795 | 3810 | GGGTTAT | GₖₛGₖₛG_{ds}T_{ds}T_{ds}A_{ds}T_{ds}A_{ds}^{m}C_{ds} | 30 | 1358 |
| | | | | | ACTCAGG | T_{ds}^{m}C_{ds}AₑₛGₖₛGₑₛ^{m}CₖₛTₑ | | |
| | | | | | CT | | | |
| 936181 | N/A | N/A | 4903 | 4918 | GCCGTCAT | Gₖₛ^{m}Cₖₛ^{m}C_{ds}G_{ds}T_{ds}^{m}C_{ds}A_{ds}T_{ds} | 46 | 1353 |
| | | | | | AATCCTG | A_{ds}A_{ds}T_{ds}^{m}Cₑₛ^{m}CₖₛTₑₛGₖₛGₑ | | |
| | | | | | G | | | |
| 936182 | N/A | N/A | 4906 | 4921 | TGCGCCGT | TₖₛGₖₛ^{m}C_{ds}G_{ds}^{m}C_{ds}^{m}C_{ds}G_{ds}T_{ds} | 81 | 1581 |
| | | | | | CATAATCC | ^{m}C_{ds}A_{ds}T_{ds}AₑₛAₖₛTₑₛ^{m}Cₖₛ^{m}Cₑ | | |
| 936183 | N/A | N/A | 4908 | 4923 | GGTGCGC | GₖₛGₖₛT_{ds}G_{ds}^{m}C_{ds}G_{ds}^{m}C_{ds}^{m}C_{ds} | 50 | 1658 |
| | | | | | CGTCATAG | _{ds}T_{ds}^{m}C_{ds}AₑₛTₖₛAₑₛAₖₛTₑ | | |
| | | | | | AT | | | |
| 936184 | N/A | N/A | 4910 | 4925 | GTGGTGC | GₖₛTₖₛG_{ds}G_{ds}T_{ds}G_{ds}^{m}C_{ds}G_{ds}^{m} | 39 | 593 |
| | | | | | GCCGTCAT | C_{ds}^{m}C_{ds}G_{ds}Tₑₛ^{m}CₖₛAₑₛTₖₛAₑ | | |
| | | | | | A | | | |
| 936185 | N/A | N/A | 5053 | 5068 | CAACTTGC | ^{m}CₖₛAₖₛA_{ds}^{m}C_{ds}T_{ds}T_{ds}G_{ds}^{m}C_{ds} | 49 | 1109 |
| | | | | | TACCCCA | T_{ds}A_{ds}^{m}C_{ds}^{m}Cₑₛ^{m}Cₖₛ^{m}CₑₛAₖₛGₑ | | |
| | | | | | G | | | |
| 936186 | N/A | N/A | 5772 | 5787 | CCCCGCAT | ^{m}Cₖₛ^{m}Cₖₛ^{m}C_{ds}^{m}C_{ds}G_{ds}^{m}C_{ds}A_{ds} | | |
| | | | | | ACGCCGT | T_{ds}A_{ds}^{m}C_{ds}G_{ds}^{m}Cₑₛ^{m}CₖₛGₑₛTₖₛ | 42 | 1707 |
| | | | | | A | Aₑ | | |
| 936218 | N/A | N/A | 3530 | 3545 | AATAAGC | AₖₛAₖₛT_{ds}A_{ds}A_{ds}G_{ds}^{m}C_{ds}A_{ds}A_{ds} | 37 | 2242 |
| | | | | | AAGTCTG | G_{ds}T_{ds}^{m}CₑₛTₖₛGₑₛGₖₛTₑ | | |
| | | | | | GT | | | |
| 936219 | N/A | N/A | 3684 | 3699 | GTTTTTGC | GₖₛTₖₛT_{ds}T_{ds}T_{ds}T_{ds}G_{ds}^{m}C_{ds}A_{ds} | 54 | 1777 |
| | | | | | ACTTCCTG | ^{m}C_{ds}T_{ds}Tₑₛ^{m}Cₖₛ^{m}CₑₛTₖₛGₑ | | |
| 936220 | N/A | N/A | 3794 | 3809 | GGTTATAC | GₖₛGₖₛT_{ds}T_{ds}A_{ds}T_{ds}A_{ds}^{m}C_{ds}T_{ds} | 50 | 2182 |
| | | | | | TCAGGCT | ^{m}C_{ds}A_{ds}GₑₛGₖₛ^{m}CₑₛTₖₛGₑ | | |
| | | | | | G | | | |
| 936221 | N/A | N/A | 4902 | 4917 | CCGTCATA | ^{m}Cₖₛ^{m}CₖₛG_{ds}T_{ds}^{m}C_{ds}A_{ds}T_{ds}A_{ds} | 39 | 1277 |
| | | | | | ATCCTGG | A_{ds}T_{ds}^{m}C_{ds}^{m}CₑₛTₖₛGₑₛGₖₛGₑ | | |
| | | | | | G | | | |
| 936222 | N/A | N/A | 4905 | 4920 | GCGCCGT | Gₖₛ^{m}CₖₛG_{ds}^{m}C_{ds}^{m}C_{ds}G_{ds}T_{ds}^{m}C_{ds} | 45 | 1505 |
| | | | | | CATAATCC | A_{ds}T_{ds}A_{ds}AₑₛTₖₛ^{m}Cₑₛ^{m}CₖₛTₑ | | |
| | | | | | T | | | |
| 936223 | N/A | N/A | 4907 | 4922 | GTGCGCC | GₖₛTₖₛG_{ds}^{m}C_{ds}G_{ds}^{m}C_{ds}^{m}C_{ds}G_{ds} | 60 | 2243 |
| | | | | | GTCATAAT | T_{ds}^{m}C_{ds}A_{ds}TₑₛAₖₛAₑₛTₖₛ^{m}Cₑ | | |
| | | | | | C | | | |
| 936224 | N/A | N/A | 4909 | 4924 | TGGTGCG | TₖₛGₖₛG_{ds}T_{ds}G_{ds}^{m}C_{ds}G_{ds}^{m}C_{ds}^{m} | 42 | 517 |
| | | | | | CCGTCATA | C_{ds}G_{ds}T_{ds}^{m}CₑₛAₖₛTₑₛAₖₛAₑ | | |
| | | | | | A | | | |
| 936225 | N/A | N/A | 5052 | 5067 | AACTTGCT | AₖₛAₖₛ^{m}C_{ds}T_{ds}T_{ds}G_{ds}^{m}C_{ds}T_{ds}A_{ds} | 55 | 1033 |
| | | | | | ACCCCAG | ^{m}C_{ds}^{m}C_{ds}^{m}Cₑₛ^{m}CₖₛAₑₛGₖₛGₑ | | |
| | | | | | G | | | |
| 936226 | N/A | N/A | 5771 | 5786 | CCCGCAT | ^{m}Cₖₛ^{m}Cₖₛ^{m}C_{ds}G_{ds}^{m}C_{ds}A_{ds}T_{ds}A_{ds} | 63 | 1513 |
| | | | | | ACGCCGT | ^{m}C_{ds}G_{ds}^{m}C_{ds}^{m}CₑₛGₖₛTₑₛAₖₛm | | |
| | | | | | AC | Cₑ | | |
| 936227 | N/A | N/A | 5773 | 5788 | GCCCCGC | Gₖₛ^{m}Cₖₛ^{m}C_{ds}^{m}C_{ds}^{m}C_{ds}G_{ds}^{m}C_{ds} | 60 | 450 |
| | | | | | ATACGCC | A_{ds}T_{ds}A_{ds}^{m}C_{ds}Gₑₛ^{m}Cₖₛ^{m}CₑₛGₖₛ | | |
| | | | | | GT | Tₑ | | |
| 936229 | N/A | N/A | 6360 | 6375 | GCAAAGG | Gₖₛ^{m}CₖₛA_{ds}A_{ds}A_{ds}G_{ds}G_{ds}^{m}C_{ds} | 41 | 2244 |
| | | | | | CATACTCC | A_{ds}T_{ds}A_{ds}^{m}CₑₛTₖₛ^{m}Cns^{m}CₖₛAₑ | | |
| | | | | | A | | | |
| 936256 | N/A | N/A | 3532 | 3547 | GCAATAA | Gₖₛ^{m}CₖₛA_{ds}A_{ds}T_{ds}A_{ds}A_{ds}G_{ds}^{m} | 29 | 2247 |
| | | | | | GCAAGTC | C_{ds}A_{ds}A_{ds}GₑₛTₖₛ^{m}CₑₛTₖₛGₑ | | |
| | | | | | TG | | | |
| 936257 | N/A | N/A | 3686 | 3701 | CGGTTTTT | ^{m}CₖₛGₖₛG_{ds}T_{ds}T_{ds}T_{ds}T_{ds}T_{ds}G_{ds} | 36 | 1928 |
| | | | | | GCACTTCC | ^{m}C_{ds}A_{ds}^{m}CₑₛTₖₛTₑₛ^{m}Cₖₛ^{m}Cₑ | | |
| 936258 | N/A | N/A | 3796 | 3811 | CGGGTTAT | ^{m}CₖₛGₖₛG_{ds}G_{ds}T_{ds}T_{ds}A_{ds}T_{ds}A_{ds} | 25 | 2183 |
| | | | | | ACTCAGG | ^{m}C_{ds}T_{ds}^{m}CnsAₖₛGₑₛGₖₛ^{m}Cₑ | | |
| | | | | | C | | | |
| 936259 | N/A | N/A | 4904 | 4919 | CGCCGTC | ^{m}CₖₛGₖₛ^{m}C_{ds}^{m}C_{ds}G_{ds}T_{ds}^{m}C_{ds}A_{ds} | 46 | 1429 |
| | | | | | ATAATCCT | T_{ds}A_{ds}A_{ds}Tₑₛ^{m}Cₖₛ^{m}CₑₛTₖₛGₑ | | |
| | | | | | G | | | |
| 936260 | N/A | N/A | 4911 | 4926 | GGTGGTG | GₖₛGₖₛT_{ds}G_{ds}G_{ds}T_{ds}G_{ds}^{m}C_{ds}G_{ds} | 45 | 669 |
| | | | | | CGCCGTC | ^{m}C_{ds}^{m}C_{ds}GₑₛTₖₛ^{m}CnsAₖₛTₑ | | |
| | | | | | AT | | | |
| 936261 | N/A | N/A | 5054 | 5069 | GCAACTT | Gₖₛ^{m}CₖₛA_{ds}A_{ds}^{m}C_{ds}T_{ds}T_{ds}G_{ds}^{m} | 46 | 1186 |
| | | | | | GCTACCCC | C_{ds}T_{ds}A_{ds}^{m}Cₑₛ^{m}Cₖₛ^{m}Cₑₛ^{m}CₖₛAₑ | | |
| | | | | | A | | | |

TABLE 33
Reduction of SPDEF RNA by 4 μM modified oligonucleotides

| | SEQ | SEQ | SEQ | SEQ | | | | |
|---|---|---|---|---|---|---|---|---|
| | ID | ID | ID | ID | | | | |
| | NO: 1 | NO: 1 | NO: 2 | NO: 2 | | | | SEQ |
| Compound | Start | Stop | Start | Stop | Sequence | Chemistry Notation | (% | ID |
| Number | Site | Site | Site | Site | (5′ to 3′) | (5′ to 3′) | UTC) | NO |
| 833814 | N/A | N/A | 12009 | 12024 | ACCAACA | Aₖₛ^{m}Cₖₛ^{m}CₖₛA_{ds}A_{ds}^{m}C_{ds}A_{ds}G_{ds} | 51 | 993 |
| | | | | | GATATAC | A_{ds}T_{ds}A_{ds}T_{ds}A_{ds}^{m}CₖₛGₖₛ^{m}C^{k} | | |
| | | | | | GC | | | |
| 854302 | N/A | N/A | 8811 | 8826 | TGGATTA | TₖₛGₖₛGₖₛA_{ds}T_{ds}T_{ds}A_{ds}A_{ds}G_{ds} | 27 | 1715 |
| | | | | | AGGCTCA | G_{ds}^{m}C_{ds}T_{ds}^{m}C_{ds}AₖₛGₖₛ^{m}C^{k} | | |
| | | | | | GC | | | |
| 936189 | N/A | N/A | 6361 | 6376 | AGCAAAG | AₖₛGₖₛ^{m}C_{ds}A_{ds}A_{ds}A_{ds}G_{ds}G_{ds}^{m} | 51 | 678 |
| | | | | | GCATACT | C_{ds}A_{ds}T_{ds}Aₑₛ^{m}CₖₛTₑₛ^{m}Cₖₛ^{m}Cₑ | | |
| | | | | | CC | | | |
| 936190 | N/A | N/A | 7439 | 7454 | CAGCATG | ^{m}CₖₛAₖₛG_{ds}^{m}C_{ds}A_{ds}T_{ds}G_{ds}A_{ds} | 65 | 1517 |
| | | | | | AGTAGAC | G_{ds}T_{ds}A_{ds}GₑₛAₖₛ^{m}CₑₛGₖₛAₑ | | |
| | | | | | GA | | | |
| 936191 | N/A | N/A | 8810 | 8825 | GGATTAA | GₖₛGₖₛA_{ds}T_{ds}T_{ds}A_{ds}A_{ds}G_{ds}G_{ds} | 28 | 683 |
| | | | | | GGCTCAG | ^{m}C_{ds}T_{ds}^{m}CₑₛAₖₛGₑₛ^{m}CₖₛGₑ | | |
| | | | | | CG | | | |
| 936192 | N/A | N/A | 8811 | 8826 | TGGATTA | TₖₛGₖₛG_{ds}A_{ds}T_{ds}T_{ds}A_{ds}A_{ds}G_{ds} | 33 | 1715 |
| | | | | | AGGCTCA | G_{ds}^{m}C_{ds}Tₑₛ^{m}CₖₛAₑₛGₖₛ^{m}Cₑ | | |
| | | | | | GC | | | |
| 936193 | N/A | N/A | 9377 | 9392 | GCTTATT | Gₖₛ^{m}CₖₛT_{ds}T_{ds}A_{ds}T_{ds}T_{ds}A_{ds}G_{ds} | 47 | 1444 |
| | | | | | AGCAGCA | ^{m}C_{ds}A_{ds}Gₑₛ^{m}CₖₛAₑₛGₖₛGₑ | | |
| | | | | | GG | | | |
| 936194 | N/A | N/A | 9801 | 9816 | GCTTTAT | Gₖₛ^{m}CₖₛT_{ds}T_{ds}T_{ds}AₖₛT_{ds}A_{ds}G_{ds} | 39 | 761 |
| | | | | | AGATGCG | A_{ds}T_{ds}Gₑₛ^{m}CₖₛGₑₛGₖₛAₑ | | |
| | | | | | GA | | | |
| 936195 | N/A | N/A | 10157 | 10172 | CCCCACC | ^{m}Cₖₛ^{m}Cₖₛ^{m}C_{ds}^{m}C_{ds}A_{ds}^{m}C_{ds}^{m}C_{ds} | 63 | 383 |
| | | | | | AAGCCTC | A_{ds}A_{ds}G_{ds}^{m}C_{ds}^{m}CₑₛTₖₛ^{m}CₑₛGₖₛ | | |
| | | | | | GG | Gₑ | | |
| 936196 | N/A | N/A | 10172 | 10187 | GCCAAGG | Gₖₛ^{m}Cₖₛ^{m}C_{ds}A_{ds}A_{ds}G_{ds}G_{ds}A_{ds} | 64 | 610 |
| | | | | | AATCTAC | A_{ds}T_{ds}^{m}C_{ds}TₑₛAₖₛ^{m}CₑₛTₖₛ^{m}Cₑ | | |
| | | | | | TC | | | |
| 936197 | N/A | N/A | 10272 | 10287 | AGAGAG | AₖₛGₖₛA_{ds}G_{ds}A_{ds}G_{ds}G_{ds}T_{ds}A_{ds} | 51 | 990 |
| | | | | | GTAAATC | A_{ds}A_{ds}Tₑₛ^{m}Cₖₛ^{m}Cₑₛ^{m}Cₖₛ^{m}Cₑ | | |
| | | | | | CCC | | | |
| 936198 | N/A | N/A | 11641 | 11656 | GACATTT | GₖₛAₖₛ^{m}C_{ds}A_{ds}T_{ds}T_{ds}T_{ds}A_{ds}T_{ds} | 38 | 1893 |
| | | | | | ATGGTGC | G_{ds}G_{ds}TₑₛGₖₛ^{m}Cₑₛ^{m}Cₖₛ^{m}Cₑ | | |
| | | | | | CC | | | |
| 936199 | N/A | N/A | 12004 | 12019 | CAGATAT | ^{m}CₖₛAₖₛG_{ds}A_{ds}T_{ds}A_{ds}T_{ds}A_{ds}^{m} | 35 | 1895 |
| | | | | | ACGCTCC | C_{ds}G_{ds}^{m}C_{ds}Tₑₛ^{m}Cₖₛ^{m}CₑₛTₖₛAₑ | | |
| | | | | | TA | | | |
| 936200 | N/A | N/A | 12006 | 12021 | AACAGAT | AₖₛAₖₛ^{m}C_{ds}A_{ds}G_{ds}A_{ds}T_{ds}A_{ds}T_{ds} | 47 | 1971 |
| | | | | | ATACGCT | A_{ds}^{m}C_{ds}Gₑₛ^{m}CₖₛTₑₛ^{m}Cₖₛ^{m}Cₑ | | |
| | | | | | CC | | | |
| 936201 | 492 | 507 | 13603 | 13618 | GCGACAC | Gₖₛ^{m}CₖₛG_{ds}A_{ds}^{m}C_{ds}A_{ds}^{m}C_{ds}^{m} | 68 | 255 |
| | | | | | CGTGTCG | C_{ds}G_{ds}T_{ds}G_{ds}Tₑₛ^{m}CₖₛGₑₛGₖₛGₑ | | |
| | | | | | GG | | | |
| 936202 | 498 | 513 | 13609 | 13624 | CTGTCCG | ^{m}CₖₛTₖₛG_{ds}T_{ds}^{m}C_{ds}^{m}C_{ds}G_{ds}^{m}C_{ds} | 57 | 1089 |
| | | | | | CGACACC | G_{ds}A_{ds}^{m}C_{ds}Aₑₛ^{m}Cₖₛ^{m}CₑₛGₖₛTₑ | | |
| | | | | | GT | | | |
| 936203 | 810 | 825 | 13921 | 13936 | GCACTTC | Gₖₛ^{m}CₖₛA_{ds}^{m}C_{ds}T_{ds}T_{ds}^{m}C_{ds}G_{ds} | 67 | 563 |
| | | | | | GCCCACC | ^{m}C_{ds}^{m}C_{ds}^{m}C_{ds}Aₑₛ^{m}Cₖₛ^{m}CₑₛAₖₛ | | |
| | | | | | AC | ^{m}Cₑ | | |
| 936204 | 829 | 844 | 13940 | 13955 | GCCGTCT | Gₖₛ^{m}Cₖₛ^{m}C_{ds}G_{ds}T_{ds}^{m}C_{ds}T_{ds}^{m}C_{ds} | 67 | 1552 |
| | | | | | CGATGTC | G_{ds}A_{ds}T_{ds}GₑₛTₖₛ^{m}Cₑₛ^{m}CₖₛTₑ | | |
| | | | | | CT | | | |
| 936206 | N/A | N/A | 14213 | 14228 | ATTCATA | AₖₛTₖₛT_{ds}^{m}C_{ds}A_{ds}T_{ds}A_{ds}T_{ds}^{m}C_{ds} | 40 | 1606 |
| | | | | | TCATCCA | A_{ds}T_{ds}^{m}Cₑₛ^{m}CₖₛAₑₛGₖₛ^{m}Cₑ | | |
| | | | | | GC | | | |
| 936207 | N/A | N/A | 14215 | 14230 | GAATTCA | GₖₛAₖₛA_{ds}T_{ds}T_{ds}^{m}C_{ds}A_{ds}T_{ds}A_{ds} | 40 | 1682 |
| | | | | | TATCATC | T_{ds}^{m}C_{ds}AₑₛTₖₛ^{m}Cₑₛ^{m}CₖₛAₑ | | |
| | | | | | CA | | | |
| 936208 | N/A | N/A | 15387 | 15402 | TGCTTTT | TₖₛGₖₛ^{m}C_{ds}T_{ds}T_{ds}T_{ds}T_{ds}^{m}C_{ds}G_{ds} | 39 | 999 |
| | | | | | CGGCCC | G_{ds}^{m}C_{ds}^{m}Cₑₛ^{m}CₖₛAₑₛGₖₛAₑ | | |
| | | | | | GA | | | |
| 936209 | N/A | N/A | 16598 | 16613 | TGAACTT | TₖₛGₖₛA_{ds}A_{ds}^{m}C_{ds}T_{ds}T_{ds}G_{ds}G_{ds} | 40 | 1686 |
| | | | | | GGTTCAG | T_{ds}T_{ds}^{m}CₑₛAₖₛGₑₛGₖₛGₑ | | |
| | | | | | GG | | | |
| 936210 | N/A | N/A | 17291 | 17306 | ACGGTTG | Aₖₛ^{m}CₖₛG_{ds}G_{ds}T_{ds}T_{ds}G_{ds}T_{ds}^{m} | 39 | 2056 |
| | | | | | TCCCCAG | C_{ds}^{m}C_{ds}^{m}C_{ds}^{m}CₑₛAₖₛGₑₛ^{m}CₖₛTₑ | | |
| | | | | | CT | | | |
| 936211 | N/A | N/A | 17292 | 17307 | CACGGTT | ^{m}CₖₛAₖₛ^{m}C_{ds}G_{ds}G_{ds}T_{ds}T_{ds}G_{ds} | 41 | 163 |
| | | | | | GTCCCCA | T_{ds}^{m}C_{ds}^{m}C_{ds}^{m}Cₑₛ^{m}CₖₛAₑₛGₖₛ^{m} | | |
| | | | | | GC | Cₑ | | |
| 936212 | N/A | N/A | 17303 | 17318 | TTCCTAG | TₖₛTₖₛ^{m}C_{ds}^{m}C_{ds}T_{ds}A_{ds}G_{ds}T_{ds} | 31 | 1754 |
| | | | | | TATCCAC | A_{ds}T_{ds}^{m}C_{ds}^{m}CₑₛAₖₛ^{m}CₑₛGₖₛGₑ | | |
| | | | | | GG | | | |
| 936213 | N/A | N/A | 17305 | 17320 | ACTTCCT | Aₖₛ^{m}CₖₛT_{ds}T_{ds}^{m}C_{ds}^{m}C_{ds}T_{ds}A_{ds} | 45 | 1906 |
| | | | | | AGTATCC | G_{ds}T_{ds}A_{ds}Tₑₛ^{m}Cₖₛ^{m}CₑₛAₖₛ^{m}Cₑ | | |
| | | | | | AC | | | |
| 936214 | N/A | N/A | 17493 | 17508 | AACTTGT | AₖₛAₖₛ^{m}C_{ds}T_{ds}T_{ds}G_{ds}T_{ds}A_{ds}A_{ds} | 21 | 2059 |
| | | | | | AACAGTG | ^{m}C_{ds}A_{ds}GₑₛTₖₛGₑₛGₖₛTₑ | | |
| | | | | | GT | | | |
| 936215 | N/A | N/A | 17525 | 17540 | TTCATAG | TₖₛTₖₛ^{m}C_{ds}A_{ds}T_{ds}A_{ds}G_{ds}A_{ds} | 65 | 240 |
| | | | | | ACTTTCC | C_{ds}T_{ds}T_{ds}Tₑₛ^{m}Cₖₛ^{m}Cₑₛ^{m}CₖₛTₑ | | |
| | | | | | CT | | | |
| 936216 | 1747 | 1762 | 20032 | 20047 | TGTCGAG | TₖₛGₖₛT_{ds}^{m}C_{ds}G_{ds}A_{ds}G_{ds}T_{ds}m | 41 | 727 |
| | | | | | TCACTGC | C_{ds}A_{ds}^{m}C_{ds}TₑₛGₖₛ^{m}Cₑₛ^{m}Cₖₛ^{m}Cₑ | | |
| | | | | | CC | | | |
| 936217 | 1894 | 1909 | 20179 | 20194 | TCTCTAG | Tₖₛ^{m}CₖₛT_{ds}^{m}C_{ds}T_{ds}A_{ds}G_{ds}T_{ds}A_{ds} | 53 | 364 |
| | | | | | TATCTTT | T_{ds}^{m}C_{ds}TₑₛTₖₛTₑₛAₖₛTₑ | | |
| | | | | | AT | | | |
| 936230 | N/A | N/A | 7438 | 7453 | AGCATGA | AₖₛGₖₛ^{m}C_{ds}A_{ds}T_{ds}G_{ds}A_{ds}G_{ds}T_{ds} | 49 | 1866 |
| | | | | | GTAGACG | A_{ds}G_{ds}Aₑₛ^{m}CₖₛGₑₛAₖₛGₑ | | |
| | | | | | AG | | | |
| 936231 | N/A | N/A | 8809 | 8824 | GATTAAG | GₖₛAₖₛT_{ds}T_{ds}A_{ds}A_{ds}G_{ds}G_{ds}^{m}C_{ds} | 42 | 2245 |
| | | | | | GCTCAGC | T_{ds}^{m}C_{ds}AₑₛGₖₛ^{m}CₑₛGₖₛTₑ | | |
| | | | | | GT | | | |
| 936232 | N/A | N/A | 9376 | 9391 | CTTATTA | ^{m}CₖₛTₖₛT_{ds}A_{ds}T_{ds}T_{ds}A_{ds}G_{ds}^{m}C_{ds} | 41 | 2221 |
| | | | | | GCAGCAG | A_{ds}G_{ds}^{m}CₑₛAₖₛGₑₛGₖₛGₑ | | |
| | | | | | GG | | | |
| 936233 | N/A | N/A | 9800 | 9815 | CTTTATA | ^{m}CₖₛTₖₛT_{ds}T_{ds}A_{ds}T_{ds}A_{ds}G_{ds}A_{ds} | 49 | 2229 |
| | | | | | GATGCGG | T_{ds}G_{ds}^{m}CₑₛGₖₛGₑₛAₖₛ^{m}Cₑ | | |
| | | | | | AC | | | |
| 936234 | N/A | N/A | 10156 | 10171 | CCCACCA | ^{m}Cₖₛ^{m}Cₖₛ^{m}C_{ds}A_{ds}^{m}C_{ds}^{m}C_{ds}A_{ds} | 49 | 2246 |
| | | | | | AGCCTCG | A_{ds}G_{ds}^{m}C_{ds}^{m}C_{ds}Tₑₛ^{m}CₖₛGₑₛGₖₛ | | |
| | | | | | GT | Tₑ | | |
| 936235 | N/A | N/A | 10171 | 10186 | CCAAGGA | ^{m}Cₖₛ^{m}CₖₛA_{ds}A_{ds}G_{ds}G_{ds}A_{ds}A_{ds} | 67 | 1734 |
| | | | | | ATCTACT | T_{ds}^{m}C_{ds}T_{ds}Aₑₛ^{m}CₖₛTₑₛ^{m}Cₖₛ^{m}Cₑ | | |
| | | | | | CC | | | |
| 936236 | N/A | N/A | 10271 | 10286 | GAGAGGT | GₖₛAₖₛG_{ds}A_{ds}G_{ds}G_{ds}T_{ds}A_{ds}A_{ds} | 47 | 2233 |
| | | | | | AAATCCC | A_{ds}T_{ds}^{m}Cₑₛ^{m}Cₖₛ^{m}Cₑₛ^{m}CₖₛAₑ | | |
| | | | | | CA | | | |
| 936237 | N/A | N/A | 11640 | 11655 | ACATTTA | Aₖₛ^{m}CₖₛA_{ds}T_{ds}T_{ds}T_{ds}A_{ds}T_{ds}G_{ds} | 45 | 992 |
| | | | | | TGGTGCC | G_{ds}T_{ds}Gₑₛ^{m}Cₖₛ^{m}Cₑₛ^{m}CₖₛTₑ | | |
| | | | | | CT | | | |
| 936238 | N/A | N/A | 12003 | 12018 | AGATATA | AₖₛGₖₛA_{ds}T_{ds}A_{ds}T_{ds}A_{ds}^{m}C_{ds}G_{ds} | 48 | 1819 |
| | | | | | CGCTCCT | ^{m}C_{ds}T_{ds}^{m}Cₑₛ^{m}CₖₛTₑₛAₖₛAₑ | | |
| | | | | | AA | | | |
| 936239 | N/A | N/A | 12005 | 12020 | ACAGATA | Aₖₛ^{m}CₖₛA_{ds}G_{ds}A_{ds}T_{ds}A_{ds}T_{ds}A_{ds} | 58 | 917 |
| | | | | | TACGCTC | ^{m}C_{ds}G_{ds}^{m}CₑₛTₖₛ^{m}Cₑₛ^{m}CₖₛTₑ | | |
| | | | | | CT | | | |
| 936240 | 491 | 506 | 13602 | 13617 | CGACACC | ^{m}CₖₛGₖₛA_{ds}^{m}Ck_{ds}A_{ds}^{m}C_{ds}^{m}C_{ds} | 69 | 2274 |
| | | | | | GTGTCGG | G_{ds}T_{ds}G_{ds}T_{ds}^{m}CₑₛGₖₛGₑₛGₖₛGₑ | | |
| | | | | | GG | | | |
| 936241 | 497 | 512 | 13608 | 13623 | TGTCCGC | TₖₛGₖₛT_{ds}^{m}C_{ds}^{m}C_{ds}G_{ds}^{m}C_{ds}G_{ds} | 56 | 1014 |
| | | | | | GACACCG | A_{ds}^{m}C_{ds}A_{ds}^{m}Cₑₛ^{m}CₖₛGₑₛTₖₛGₑ | | |
| | | | | | TG | | | |
| 936242 | 809 | 824 | 13920 | 13935 | CACTTCG | ^{m}CₖₛAₖₛ^{m}C_{ds}T_{ds}T_{ds}^{m}C_{ds}G_{ds}^{m}C_{ds} | 52 | 487 |
| | | | | | CCCACCA | ^{m}C_{ds}^{m}C_{ds}A_{ds}^{m}Cₑₛ^{m}CₖₛAₑₛ^{m}Cₖₛ | | |
| | | | | | CC | ^{m}Cₑ | | |
| 936243 | 828 | 843 | 13939 | 13954 | CCGTCTC | ^{m}Cₖₛ^{m}CₖₛG_{ds}T_{ds}^{m}C_{ds}T_{ds}^{m}C_{ds}G_{ds} | 51 | 37 |
| | | | | | GATGTCC | A_{ds}T_{ds}G_{ds}Tₑₛ^{m}Cₖₛ^{m}CₑₛTₖₛTₑ | | |
| | | | | | TT | | | |
| 936245 | N/A | N/A | 14212 | 14227 | TTCATAT | TₖₛTₖₛ^{m}C_{ds}A_{ds}T_{ds}A_{ds}T_{ds}^{m}C_{ds}A_{ds} | 41 | 2275 |
| | | | | | CATCCAG | T_{ds}^{m}C_{ds}^{m}CₑₛAₖₛGₑₛ^{m}CₖₛAₑ | | |
| | | | | | CA | | | |
| 936246 | N/A | N/A | 14214 | 14229 | AATTCAT | AₖₛAₖₛT_{ds}T_{ds}^{m}C_{ds}A_{ds}T_{ds}A_{ds}T_{ds} | 50 | 2255 |
| | | | | | ATCATCC | ^{m}C_{ds}A_{ds}Tₑₛ^{m}Cₖₛ^{m}CₑₛAₖₛGₑ | | |
| | | | | | AG | | | |
| 936247 | N/A | N/A | 15386 | 15401 | GCTTTTC | Gₖₛ^{m}CₖₛT_{ds}T_{ds}T_{ds}T_{ds}^{m}C_{ds}G_{ds}G_{ds} | 32 | 2263 |
| | | | | | GGCCCAG | ^{m}C_{ds}^{m}C_{ds}^{m}CₑₛAₖₛGₑₛAₖₛGₑ | | |
| | | | | | AG | | | |
| 936248 | N/A | N/A | 16597 | 16612 | GAACTTG | GₖₛAₖₛA_{ds}^{m}C_{ds}T_{ds}T_{ds}G_{ds}G_{ds}T_{ds} | 60 | 2276 |
| | | | | | GTTCAGG | T_{ds}^{m}C_{ds}AₑₛGₖₛGₑₛGₖₛ^{m}Cₑ | | |
| | | | | | GC | | | |
| 936249 | N/A | N/A | 17290 | 17305 | CGGTTGT | ^{m}CₖₛGₖₛG_{ds}T_{ds}T_{ds}G_{ds}T_{ds}^{m}C_{ds}^{m} | 28 | 2277 |
| | | | | | CCCCAGC | C_{ds}^{m}C_{ds}^{m}C_{ds}AₑₛGₖₛ^{m}CₑₛTₖₛ^{m}Cₑ | | |
| | | | | | TC | | | |
| 936250 | N/A | N/A | 17302 | 17317 | TCCTAGT | Tₖₛ^{m}Cₖₛ^{m}C_{ds}T_{dsds}G_{ds}T_{ds}A_{ds}T_{ds} | 43 | 1230 |
| | | | | | ATCCACG | ^{m}C_{ds}^{m}C_{ds}Aₑₛ^{m}CₖₛGₑₛGₖₛTₑ | | |
| | | | | | GT | | | |
| 936251 | N/A | N/A | 17304 | 17319 | CTTCCTA | ^{m}CₖₛTₖₛT_{ds}^{m}C_{ds}^{m}C_{ds}T_{ds}A_{ds}G_{ds} | 35 | 1830 |
| | | | | | GTATCCA | T_{ds}A_{ds}T_{ds}^{m}Cₑₛ^{m}CₖₛAₑₛ^{m}CₖₛGₑ | | |
| | | | | | CG | | | |
| 936252 | N/A | N/A | 17492 | 17507 | ACTTGTA | Aₖₛ^{m}CₖₛT_{ds}T_{ds}G_{ds}T_{ds}A_{ds}A_{ds}^{m} | 38 | 1983 |
| | | | | | ACAGTGG | C_{ds}A_{ds}G_{ds}TₑₛGₖₛGₑₛTₖₛTₑ | | |
| | | | | | TT | | | |
| 936253 | N/A | N/A | 17524 | 17539 | TCATAGA | Tₖₛ^{m}CₖₛA_{ds}T_{ds}A_{ds}G_{ds}A_{ds}^{m}C_{ds} | 50 | 2278 |
| | | | | | CTTTCCC | T_{ds}T_{ds}T_{ds}^{m}Cₑₛ^{m}Cₖₛ^{m}CₑₛTₖₛGₑ | | |
| | | | | | TG | | | |
| 936254 | 1746 | 1761 | 20031 | 20046 | GTCGAGT | GₖₛTₖₛ^{m}C_{ds}G_{ds}A_{ds}G_{ds}T_{ds}^{m}C_{ds} | 55 | 58 |
| | | | | | CACTGCC | A_{ds}^{m}C_{ds}T_{ds}Gₑₛ^{m}Cₖₛ^{m}Cₑₛ^{m}CₖₛTₑ | | |
| | | | | | CT | | | |
| 936255 | 1893 | 1908 | 20178 | 20193 | CTCTAGT | ^{m}CₖₛTₖₛ^{m}C_{ds}T_{ds}A_{ds}G_{ds}T_{ds}A_{ds}T_{ds} | 56 | 2279 |
| | | | | | ATCTTTA | ^{m}C_{ds}T_{ds}TₑₛTₖₛAₑₛTₖₛTₑ | | |
| | | | | | TT | | | |
| 936264 | N/A | N/A | 6362 | 6377 | AAGCAAA | AₖₛAₖₛG_{ds}^{m}C_{ds}A_{ds}A_{ds}A_{ds}G_{ds}G_{ds} | 46 | 2248 |
| | | | | | GGCATAC | ^{m}C_{ds}A_{ds}TₑₛAₖₛ^{m}CₑₛTₖₛ^{m}Cₑ | | |
| | | | | | TC | | | |
| 936265 | N/A | N/A | 7440 | 7455 | TCAGCAT | Tₖₛ^{m}CₖₛA_{ds}G_{ds}^{m}C_{ds}A_{ds}T_{ds}G_{ds} | 55 | 1942 |
| | | | | | GAGTAGA | A_{ds}G_{ds}T_{ds}AₑₛGₖₛAₑₛ^{m}CₖₛGₑ | | |
| | | | | | CG | | | |
| 936266 | N/A | N/A | 8812 | 8827 | CTGGATT | ^{m}CₖₛTₖₛG_{ds}G_{ds}A_{ds}T_{ds}T_{ds}A_{ds}A_{ds} | 56 | 759 |
| | | | | | AAGGCTC | G_{ds}G_{ds}^{m}CₑₛTₖₛ^{m}CₑₛAₖₛGₑ | | |
| | | | | | AG | | | |
| 936267 | N/A | N/A | 9378 | 9393 | TGCTTAT | TₖₛGₖₛ^{m}C_{ds}T_{ds}T_{ds}A_{ds}T_{ds}T_{ds}A_{ds} | 73 | 2222 |
| | | | | | TAGCAGC | G_{ds}^{m}C_{ds}AₑₛGₖₛ^{m}CₑₛAₖₛGₑ | | |
| | | | | | AG | | | |
| 936268 | N/A | N/A | 9802 | 9817 | TGCTTTA | TₖₛGₖₛ^{m}C_{ds}T_{ds}T_{ds}T_{ds}A_{ds}T_{ds}A_{ds} | 38 | 2230 |
| | | | | | TAGATGC | G_{ds}A_{ds}TₑₛGₖₛ^{m}CₑₛGₖₛGₑ | | |
| | | | | | GG | | | |
| 936269 | N/A | N/A | 10158 | 10173 | TCCCCAC | Tₖₛ^{m}Cₖₛ^{m}C_{ds}^{m}C_{ds}^{m}C_{ds}A_{ds}^{m}C_{ds} | 69 | 2249 |
| | | | | | CAAGCCT | ^{m}C_{ds}A_{ds}A_{ds}G_{ds}^{m}Cₑₛ^{m}CₖₛTₑₛ^{m}Cₖₛ | | |
| | | | | | CG | Gₑ | | |
| 936270 | N/A | N/A | 10173 | 10188 | TGCCAAG | TₖₛGₖₛ^{m}C_{ds}^{m}C_{ds}A_{ds}A_{ds}G_{ds}G_{ds} | 63 | 1810 |
| | | | | | GAATCTA | A_{ds}A_{ds}T_{ds}^{m}CₑₛTₖₛAₑₛ^{m}CₖₛTₑ | | |
| | | | | | CT | | | |
| 936271 | N/A | N/A | 10273 | 10288 | CAGAGAG | ^{m}CₖₛAₖₛG_{ds}A_{ds}G_{ds}G_{ds}A_{ds}G_{ds}G_{ds} | 69 | 2234 |
| | | | | | GTAAATC | T_{ds}A_{ds}A_{ds}AₑₛTₖₛ^{m}Cₑₛ^{m}Cₖₛ^{m}Cₑ | | |
| | | | | | CC | | | |
| 936272 | N/A | N/A | 11642 | 11657 | TGACATT | TₖₛGₖₛA_{ds}^{m}C_{ds}A_{ds}T_{ds}T_{ds}T_{ds}A_{ds} | 66 | 1068 |
| | | | | | TATGGTG | T_{ds}G_{ds}GₑₛTₖₛGₑₛ^{m}Cₖₛ^{m}Cₑ | | |
| | | | | | CC | | | |
| 936273 | N/A | N/A | 12007 | 12022 | CAACAGA | ^{m}CₖₛAₖₛA_{ds}^{m}C_{ds}A_{ds}G_{ds}A_{ds}T_{ds} | 34 | 2047 |
| | | | | | TATACGC | A_{ds}T_{ds}A_{ds}^{m}CₑₛGₖₛ^{m}CₑₛTₖₛ^{m}Cₑ | | |
| | | | | | TC | | | |
| 936274 | 493 | 508 | 13604 | 13619 | CGCGACA | ^{m}CₖₛGₖₛ^{m}C_{ds}G_{ds}A_{ds}^{m}C_{ds}A_{ds}^{m} | 64 | 861 |
| | | | | | CCGTGTC | C_{ds}^{m}C_{ds}G_{ds}T_{ds}GₑₛTₖₛ^{m}CₑₛGₖₛ | | |
| | | | | | GG | Gₑ | | |
| 936275 | 499 | 514 | 13610 | 13625 | CCTGTCC | ^{m}Cₖₛ^{m}CₖₛT_{ds}G_{ds}T_{ds}^{m}C_{ds}^{m}C_{ds}G_{ds} | 48 | 1165 |
| | | | | | GCGACAC | ^{m}C_{ds}G_{ds}A_{ds}^{m}CₑₛAₖₛ^{m}Cₑₛ^{m}Cₖₛ | | |
| | | | | | CG | Gₑ | | |
| 936276 | 811 | 826 | 13922 | 13937 | AGCACTT | AₖₛGₖₛ^{m}C_{ds}A_{ds}^{m}C_{ds}T_{ds}T_{ds}^{m}C_{ds} | 57 | 640 |
| | | | | | CGCCCAC | G_{ds}^{m}C_{ds}^{m}C_{ds}^{m}CₑₛAₖₛ^{m}Cₑₛ^{m}Cₖₛ | | |
| | | | | | CA | Aₑ | | |
| 936277 | 830 | 845 | 13941 | 13956 | GGCCGTC | GₖₛGₖₛ^{m}C_{ds}^{m}C_{ds}G_{ds}T_{ds}^{m}C_{ds}T_{ds} | 66 | 114 |
| | | | | | TCGATGT | ^{m}C_{ds}G_{ds}A_{ds}TₑₛGₖₛTₑₛ^{m}Cₖₛ^{m}Cₑ | | |
| | | | | | CC | | | |
| 936279 | N/A | N/A | 14216 | 14231 | TGAATTC | TₖₛGₖₛA_{ds}A_{ds}T_{ds}T_{ds}^{m}C_{ds}A_{ds}T_{ds} | 34 | 2280 |
| | | | | | ATATCAT | A_{ds}T_{ds}^{m}CₑₛAₖₛTₑₛ^{m}Cₖₛ^{m}Cₑ | | |
| | | | | | CC | | | |
| 936280 | N/A | N/A | 15388 | 15403 | CTGCTTT | ^{m}CₖₛTₖₛG_{ds}^{m}C_{ds}T_{ds}T_{ds}T_{ds}T_{ds}^{m} | 50 | 2264 |
| | | | | | TCGGCCC | C_{ds}G_{ds}G_{ds}^{m}Cₑₛ^{m}Cₖₛ^{m}CₑₛAₖₛGₑ | | |
| | | | | | AG | | | |
| 936281 | N/A | N/A | 16599 | 16614 | ATGAACT | AₖₛTₖₛG_{ds}A_{ds}A_{ds}^{m}C_{ds}T_{ds}T_{ds}G_{ds} | 85 | 2281 |
| | | | | | TGGTTCA | G_{ds}T_{ds}Tₑₛ^{m}CₖₛAₑₛGₖₛGₑ | | |
| | | | | | GG | | | |
| 936282 | N/A | N/A | 17293 | 17308 | CCACGGT | ^{m}Cₖₛ^{m}CₖₛA_{ds}^{m}C_{ds}G_{ds}G_{ds}T_{ds}T_{ds} | 73 | 2132 |
| | | | | | TGTCCCC | G_{ds}T_{ds}^{m}C_{ds}^{m}Cₑₛ^{m}Cₖₛ^{m}CₑₛAₖₛGₑ | | |
| | | | | | AG | | | |
| 936283 | N/A | N/A | 17306 | 17321 | GACTTCC | GₖₛAₖₛ^{m}C_{ds}T_{ds}T_{ds}^{m}C_{ds}^{m}C_{ds}T_{ds} | 59 | 1892 |
| | | | | | TAGTATC | A_{ds}G_{ds}T_{ds}AₑₛTₖₛ^{m}Cₑₛ^{m}CₖₛAₑ | | |
| | | | | | CA | | | |
| 936284 | N/A | N/A | 17494 | 17509 | AAACTTG | AₖₛAₖₛA_{ds}^{m}C_{ds}T_{ds}T_{ds}G_{ds}T_{ds}A_{ds} | 33 | 2282 |
| | | | | | TAACAGT | A_{ds}^{m}C_{ds}AₑₛGₖₛTₑₛGₖₛGₑ | | |
| | | | | | GG | | | |
| 936285 | N/A | N/A | 17526 | 17541 | ATTCATA | AₖₛTₖₛT_{ds}^{m}C_{ds}A_{ds}T_{ds}A_{ds}G_{ds}A_{ds} | 45 | 2283 |
| | | | | | GACTTTC | ^{m}C_{ds}T_{ds}TₑₛTₖₛ^{m}Cₑₛ^{m}Cₖₛ^{m}Cₑ | | |
| | | | | | CC | | | |
| 936286 | 1748 | 1763 | 20033 | 20048 | TTGTCGA | TₖₛTₖₛG_{ds}T_{ds}^{m}C_{ds}G_{ds}A_{ds}G_{ds}T_{ds} | 38 | 803 |
| | | | | | GTCACTG | ^{m}C_{ds}A_{ds}^{m}CₑₛTₖₛGₑₛ^{m}Cₖₛ^{m}Cₑ | | |
| | | | | | CC | | | |
| 936287 | 1895 | 1910 | 20180 | 20195 | TTCTCTA | TₖₛTₖₛ^{m}C_{ds}T_{ds}^{m}C_{ds}T_{ds}A_{ds}G_{ds} | 43 | 2284 |
| | | | | | GTATCTT | T_{ds}A_{ds}T_{ds}^{m}CₑₛTₖₛTₑₛTₖₛAₑ | | |
| | | | | | TA | | | |

### Example 3: Effect of Modified Oligonucleotides on Human SPDEF RNA In Vitro, Multiple Doses

Modified oligonucleotides selected from the examples above were tested at various doses in VCaP cells. Cultured VCaP cells at a density of 20,000 cells per well were treated with modified oligonucleotide at various doses by electroporation, as specified in the tables below. After a treatment period of approximately 24 hours, total RNA was isolated from the cells and SPDEF RNA levels were measured by quantitative real-time RTPCR. Human SPDEF primer probe set RTS35007 was used to measure RNA levels as described above. SPDEF RNA levels were normalized to total RNA content, as measured by RIBOGREEN®. Results are presented in the tables below as percent reduction of the amount of SPDEF RNA, relative to untreated control (UTC). The half maximal inhibitory concentration (IC₅₀) of each modified oligonucleotide is also presented. IC₅₀ was calculated using a linear regression on a log/linear plot of the data in Excel.

TABLE 34
Dose-dependent percent reduction of human
SPDEF RNA by modified oligonucleotides

| Compound | SPDEF (% UTC) | | | | |
|---|---|---|---|---|---|
| Number | 444 nM | 1333 nM | 4000 nM | 12000 nM | IC₅₀ μM |
| 652522 | 98 | 79 | 54 | 31 | 4.9 |
| 801690 | 86 | 109 | 83 | 53 | >12 |
| 801727 | 86 | 74 | 57 | 33 | 5.0 |
| 801803 | 108 | 93 | 72 | 46 | 11.0 |
| 801850 | 95 | 89 | 60 | 35 | 6.5 |
| 801894 | 84 | 77 | 67 | 35 | 6.9 |
| 801919 | 83 | 66 | 53 | 30 | 3.8 |
| 801930 | 93 | 115 | 92 | 57 | >12 |
| 801946 | 76 | 55 | 41 | 21 | 2.1 |
| 801965 | 84 | 66 | 58 | 23 | 3.6 |
| 801972 | 98 | 73 | 65 | 35 | 6.2 |
| 801974 | 96 | 82 | 64 | 37 | 6.9 |
| 802029 | 93 | 84 | 63 | 45 | 9.5 |
| 802032 | 83 | 74 | 59 | 29 | 4.6 |
| 802055 | 101 | 89 | 76 | 63 | >12 |
| 802075 | 109 | 109 | 88 | 59 | >12 |
| 802094 | 74 | 50 | 43 | 26 | 2.1 |
| 802095 | 85 | 69 | 51 | 14 | 2.9 |
| 802103 | 90 | 83 | 81 | 68 | >12 |

TABLE 35
Dose-dependent percent reduction of human
SPDEF RNA by modified oligonucleotides

| Compound | SPDEF (% UTC) | | | | |
|---|---|---|---|---|---|
| Number | 234 nM | 938 nM | 3750 nM | 15000 nM | IC₅₀ μM |
| 801766 | 82 | 64 | 37 | 22 | 2.1 |
| 802094 | 82 | 45 | 23 | 13 | 1.1 |
| 832871 | 111 | 88 | 60 | 37 | 7.1 |
| 832904 | 100 | 70 | 37 | 35 | 3.5 |
| 832905 | 88 | 73 | 43 | 26 | 3.1 |
| 832967 | 113 | 83 | 46 | 36 | 5.1 |
| 833000 | 95 | 68 | 46 | 21 | 2.9 |
| 833201 | 65 | 93 | 66 | 28 | 6.3 |
| 833202 | 103 | 67 | 39 | 39 | 4.0 |
| 833266 | 92 | 89 | 69 | 32 | 7.6 |
| 833489 | 97 | 56 | 52 | 47 | 6.0 |
| 833490 | 93 | 58 | 50 | 19 | 2.6 |
| 833601 | 107 | 76 | 51 | 46 | 7.1 |
| 833635 | 83 | 61 | 40 | 17 | 1.9 |
| 833683 | 108 | 100 | 73 | 40 | 11.6 |
| 833715 | 108 | 69 | 39 | 28 | 3.3 |
| 833762 | 89 | 71 | 45 | 25 | 3.0 |
| 833825 | 129 | 111 | 74 | 51 | >15 |
| 833904 | 91 | 68 | 45 | 38 | 4.1 |

TABLE 36
Dose-dependent percent reduction of human
SPDEF RNA by modified oligonucleotides

| Compound | SPDEF (% UTC) | | | | |
|---|---|---|---|---|---|
| Number | 234 nM | 938 nM | 3750 nM | 15000 nM | IC₅₀ μM |
| 802094 | 89 | 46 | 31 | 10 | 1.4 |
| 832988 | 89 | 82 | 48 | 32 | 4.4 |
| 833123 | 70 | 64 | 38 | 27 | 1.8 |
| 833188 | 79 | 72 | 38 | 36 | 3.2 |
| 833204 | 138 | 86 | 66 | 69 | >15 |
| 833270 | 112 | 73 | 47 | 32 | 4.3 |
| 833366 | 92 | 94 | 52 | 36 | 6.3 |
| 833413 | 98 | 81 | 46 | 27 | 3.9 |
| 833491 | 83 | 67 | 34 | 31 | 2.5 |
| 833572 | 89 | 56 | 31 | 25 | 1.9 |
| 833699 | 93 | 68 | 51 | 31 | 3.9 |
| 833733 | 95 | 71 | 60 | 26 | 4.3 |
| 833748 | 126 | 89 | 30 | 16 | 3.2 |
| 833780 | 89 | 70 | 51 | 22 | 3.1 |
| 833813 | 123 | 91 | 30 | 23 | 3.6 |
| 833907 | 101 | 81 | 50 | 32 | 4.8 |
| 833923 | 112 | 106 | 86 | 59 | >15 |
| 833939 | 86 | 97 | 73 | 64 | >15 |
| 833973 | 113 | 87 | 54 | 21 | 4.4 |

TABLE 37
Dose-dependent percent reduction of human
SPDEF RNA by modified oligonucleotides

| Compound | SPDEF (% UTC) | | | | |
|---|---|---|---|---|---|
| Number | 234 nM | 938 nM | 3750 nM | 15000 nM | IC₅₀ μM |
| 802094 | 83 | 51 | 37 | 14 | 1.5 |
| 832911 | 73 | 70 | 49 | 28 | 3.0 |
| 832989 | 91 | 80 | 56 | 19 | 3.6 |
| 833240 | 91 | 78 | 50 | 39 | 5.8 |
| 833241 | 90 | 72 | 48 | 25 | 3.2 |
| 833336 | 81 | 51 | 30 | 13 | 1.3 |
| 833350 | 117 | 91 | 62 | 50 | 11.0 |
| 833401 | 86 | 68 | 55 | 32 | 4.2 |
| 833416 | 85 | 80 | 56 | 32 | 5.1 |
| 833561 | 84 | 64 | 39 | 25 | 2.3 |
| 833575 | 82 | 56 | 29 | 16 | 1.5 |
| 833609 | 87 | 89 | 57 | 35 | 6.7 |
| 833767 | 79 | 61 | 41 | 16 | 1.8 |
| 833799 | 102 | 78 | 50 | 38 | 5.5 |
| 833814 | 102 | 76 | 47 | 24 | 3.6 |
| 833816 | 101 | 81 | 48 | 36 | 5.1 |
| 833849 | 102 | 88 | 69 | 56 | >15 |
| 833910 | 81 | 60 | 34 | 19 | 1.7 |
| 833911 | 77 | 51 | 30 | 23 | 1.3 |

TABLE 38
Dose-dependent percent reduction of human
SPDEF RNA by modified oligonucleotides

| Compound | SPDEF (% UTC) | | | | |
|---|---|---|---|---|---|
| Number | 234 nM | 938 nM | 3750 nM | 15000 nM | IC₅₀ μM |
| 802094 | 88 | 55 | 38 | 21 | 1.9 |
| 832881 | 102 | 95 | 94 | 94 | >15 |
| 833041 | 117 | 103 | 100 | 98 | >15 |
| 833211 | 94 | 74 | 46 | 31 | 3.9 |
| 833242 | 46 | 61 | 35 | 14 | 1.7 |
| 833243 | 100 | 69 | 39 | 35 | 3.6 |
| 833276 | 91 | 75 | 50 | 21 | 3.3 |
| 833338 | 107 | 83 | 52 | 29 | 4.7 |
| 833340 | 101 | 86 | 58 | 37 | 6.9 |
| 833419 | 86 | 88 | 66 | 37 | 9.1 |
| 833484 | 92 | 70 | 51 | 22 | 3.2 |
| 833579 | 78 | 61 | 45 | 14 | 1.9 |
| 833580 | 75 | 54 | 36 | 20 | 1.4 |
| 833581 | 99 | 70 | 53 | 30 | 4.2 |
| 833738 | 91 | 96 | 76 | 46 | 13.0 |
| 833756 | 99 | 76 | 48 | 30 | 4.2 |
| 833773 | 94 | 68 | 50 | 32 | 3.9 |
| 833882 | 86 | 75 | 47 | 26 | 3.3 |
| 833962 | 99 | 92 | 68 | 42 | 11.0 |

TABLE 39
Dose-dependent percent reduction of human
SPDEF RNA by modified oligonucleotides

| Compound | SPDEF (% UTC) | | | | |
|---|---|---|---|---|---|
| Number | 234 nM | 938 nM | 3750 nM | 15000 nM | IC₅₀ μM |
| 802094 | 86 | 50 | 26 | 13 | 1.3 |
| 833013 | 83 | 70 | 43 | 20 | 2.4 |
| 833117 | 76 | 81 | 76 | 77 | >15 |
| 833198 | 84 | 77 | 47 | 34 | 4.3 |
| 833277 | 88 | 73 | 56 | 27 | 4.1 |
| 833343 | 102 | 76 | 51 | 28 | 4.2 |
| 833486 | 99 | 85 | 54 | 24 | 4.3 |
| 833487 | 80 | 64 | 42 | 29 | 2.6 |
| 833567 | 75 | 59 | 38 | 17 | 1.5 |
| 833631 | 97 | 75 | 52 | 19 | 3.4 |
| 833678 | 76 | 72 | 46 | 28 | 2.9 |
| 833741 | 84 | 62 | 37 | 21 | 2.0 |
| 833758 | 99 | 77 | 63 | 35 | 6.5 |
| 833868 | 110 | 93 | 79 | 48 | >15 |
| 833886 | 101 | 62 | 47 | 22 | 2.9 |
| 833901 | 98 | 69 | 66 | 24 | 4.6 |
| 833932 | 84 | 70 | 45 | 31 | 3.2 |
| 833965 | 85 | 74 | 48 | 34 | 4.2 |
| 833980 | 76 | 83 | 63 | 44 | 13.0 |

TABLE 40
Dose-dependent percent reduction of human
SPDEF RNA by modified oligonucleotides

| Compound | SPDEF (% UTC) | | | | |
|---|---|---|---|---|---|
| Number | 234 nM | 938 nM | 3750 nM | 15000 nM | IC₅₀ μM |
| 802094 | 93 | 67 | 26 | 15 | 1.9 |
| 833014 | 88 | 94 | 99 | 77 | >15 |
| 833488 | 73 | 57 | 40 | 25 | 1.7 |
| 833536 | 95 | 79 | 67 | 58 | >15 |
| 833711 | 89 | 76 | 58 | 25 | 4.1 |
| 833887 | 71 | 60 | 39 | 22 | 1.6 |
| 833951 | 77 | 70 | 51 | 29 | 3.3 |
| 854182 | 97 | 81 | 55 | 34 | 5.6 |
| 854254 | 77 | 69 | 46 | 14 | 2.1 |
| 854255 | 96 | 76 | 50 | 21 | 3.4 |
| 854302 | 66 | 52 | 35 | 12 | 0.9 |
| 854337 | 75 | 50 | 50 | 16 | 1.6 |
| 854355 | 87 | 74 | 45 | 33 | 3.7 |
| 854452 | 93 | 67 | 47 | 31 | 3.5 |
| 854535 | 79 | 67 | 42 | 12 | 1.9 |
| 854547 | 104 | 92 | 57 | 32 | 6.2 |
| 854577 | 83 | 70 | 46 | 25 | 2.8 |
| 854589 | 78 | 68 | 45 | 22 | 2.3 |
| 854608 | 101 | 74 | 65 | 43 | 9.1 |

TABLE 41
Dose-dependent percent reduction of human
SPDEF RNA by modified oligonucleotides

| Compound | SPDEF (% UTC) | | | | |
|---|---|---|---|---|---|
| Number | 234 nM | 938 nM | 3750 nM | 15000 nM | IC₅₀ μM |
| 802094 | 92 | 59 | 32 | 19 | 1.9 |
| 854183 | 86 | 95 | 68 | 33 | 7.4 |
| 854196 | 100 | 70 | 41 | 24 | 3.0 |
| 854214 | 80 | 69 | 42 | 21 | 2.3 |
| 854215 | 81 | 59 | 46 | 18 | 2.0 |
| 854226 | 89 | 68 | 29 | 14 | 1.9 |
| 854303 | 95 | 78 | 54 | 27 | 4.3 |
| 854338 | 85 | 76 | 52 | 17 | 2.9 |
| 854393 | 77 | 64 | 43 | 26 | 2.3 |
| 854398 | 96 | 75 | 64 | 23 | 4.5 |
| 854453 | 103 | 77 | 49 | 33 | 4.6 |
| 854458 | 88 | 90 | 79 | 50 | >15 |
| 854459 | 80 | 52 | 32 | 17 | 1.4 |
| 854471 | 78 | 68 | 44 | 12 | 1.9 |
| 854472 | 85 | 63 | 38 | 24 | 2.2 |
| 854519 | 91 | 77 | 53 | 23 | 3.6 |
| 854537 | 83 | 63 | 51 | 27 | 3.0 |
| 854538 | 73 | 70 | 37 | 35 | 2.7 |
| 854544 | 97 | 75 | 47 | 35 | 4.5 |

TABLE 42
Dose-dependent percent reduction of human
SPDEF RNA by modified oligonucleotides

| Compound | SPDEF (% UTC) | | | | |
|---|---|---|---|---|---|
| Number | 234 nM | 938 nM | 3750 nM | 15000 nM | IC₅₀ μM |
| 802094 | 84 | 66 | 37 | 25 | 2.3 |
| 854204 | 94 | 85 | 60 | 40 | 8.3 |
| 854211 | 115 | 106 | 93 | 59 | >15 |
| 854216 | 79 | 87 | 52 | 36 | 5.8 |
| 854227 | 87 | 62 | 39 | 17 | 2.0 |
| 854234 | 123 | 108 | 89 | 49 | >15 |
| 854235 | 89 | 85 | 49 | 37 | 5.4 |
| 854252 | 90 | 82 | 59 | 43 | 9.0 |
| 854288 | 108 | 92 | 63 | 41 | 8.8 |
| 854340 | 77 | 72 | 44 | 31 | 3.1 |
| 854353 | 93 | 84 | 64 | 56 | >15 |
| 854360 | 101 | 95 | 84 | 50 | >15 |
| 854376 | 89 | 88 | 50 | 19 | 3.5 |
| 854390 | 90 | 78 | 48 | 36 | 4.9 |
| 854486 | 99 | 97 | 78 | 66 | >15 |
| 854526 | 68 | 60 | 41 | 17 | 1.4 |
| 854527 | 95 | 64 | 38 | 12 | 2.1 |
| 854545 | 84 | 85 | 40 | 11 | 2.5 |
| 854575 | 95 | 91 | 63 | 28 | 6.1 |

TABLE 43
Dose-dependent percent reduction of human SPDEF
RNA by modified oligonucleotides

| Compound | SPDEF (% UTC) | | | | | IC₅₀ |
|---|---|---|---|---|---|---|
| Number | 148 nM | 444 nM | 1333 nM | 4000 nM | 12000 nM | μM |
| 801683 | 87 | 75 | 73 | 71 | 44 | >12 |
| 801766 | 101 | 100 | 73 | 48 | 33 | 4.6 |
| 801808 | 104 | 87 | 69 | 42 | 30 | 3.4 |
| 801907 | 125 | 110 | 85 | 51 | 20 | 4.3 |
| 801909 | 86 | 67 | 50 | 17 | 17 | 1.1 |
| 801950 | 128 | 94 | 50 | 31 | 18 | 2.3 |
| 801958 | 96 | 88 | 67 | 44 | 21 | 2.8 |
| 801983 | 112 | 98 | 81 | 68 | 39 | 8.6 |
| 802030 | 50 | 96 | 78 | 53 | 28 | 4.5 |
| 802043 | 104 | 86 | 49 | 30 | 14 | 1.8 |
| 802048 | 107 | 101 | 70 | 54 | 42 | 6.1 |
| 802053 | 120 | 110 | 89 | 61 | 35 | 7.0 |
| 802094 | 81 | 59 | 52 | 25 | 23 | 1.2 |
| 802098 | 103 | 80 | 47 | 24 | 12 | 1.5 |

TABLE 44
Dose-dependent percent reduction of human
SPDEF RNA by modified oligonucleotides

| Compound | SPDEF (% UTC) | | | | |
|---|---|---|---|---|---|
| Number | 556 nM | 1667 nM | 5000 nM | 15000 nM | IC₅₀ μM |
| 833814 | 88 | 56 | 41 | 18 | 3.0 |
| 854302 | 71 | 51 | 22 | 15 | 1.6 |
| 936070 | 60 | 29 | 23 | 10 | 0.7 |
| 936110 | 82 | 58 | 29 | 33 | 3.0 |
| 936148 | 51 | 39 | 29 | 19 | 0.6 |
| 936292 | 69 | 61 | 52 | 30 | 3.8 |
| 936299 | 75 | 61 | 38 | 19 | 2.6 |
| 936301 | 72 | 73 | 38 | 33 | 3.9 |
| 936310 | 71 | 49 | 36 | 20 | 2.0 |
| 936315 | 71 | 61 | 46 | 21 | 2.9 |
| 936316 | 66 | 57 | 36 | 21 | 2.0 |
| 936317 | 86 | 70 | 50 | 36 | 5.7 |
| 936336 | 99 | 77 | 83 | 25 | 8.3 |
| 936381 | 66 | 55 | 31 | 21 | 1.8 |
| 936396 | 73 | 43 | 38 | 27 | 2.0 |
| 936415 | 69 | 56 | 48 | 14 | 2.4 |
| 936416 | 74 | 40 | 54 | 24 | 2.5 |
| 936421 | 87 | 88 | 78 | 38 | 14.4 |
| 936429 | 79 | 65 | 40 | 32 | 3.8 |

TABLE 45
Dose-dependent percent reduction of human
SPDEF RNA by modified oligonucleotides

| Compound | SPDEF (% UTC) | | | | |
|---|---|---|---|---|---|
| Number | 556 nM | 1667 nM | 5000 nM | 15000 nM | IC₅₀ μM |
| 833814 | 74 | 59 | 37 | 24 | 2.6 |
| 854302 | 68 | 39 | 20 | 17 | 1.2 |
| 936068 | 78 | 65 | 48 | 37 | 5.1 |
| 936069 | 78 | 62 | 47 | 17 | 3.1 |
| 936081 | 66 | 42 | 27 | 14 | 1.3 |
| 936082 | 67 | 51 | 35 | 27 | 1.9 |
| 936088 | 73 | 60 | 29 | 20 | 2.2 |
| 936111 | 100 | 87 | 66 | 37 | 9.4 |
| 936121 | 78 | 59 | 43 | 24 | 3.1 |
| 936135 | 93 | 62 | 38 | 38 | 4.5 |
| 936142 | 67 | 47 | 27 | 13 | 1.4 |
| 936147 | 71 | 54 | 35 | 20 | 2.1 |
| 936150 | 72 | 57 | 43 | 24 | 2.7 |
| 936158 | 68 | 56 | 31 | 28 | 2.1 |
| 936258 | 67 | 34 | 21 | 10 | 1.0 |
| 936442 | 64 | 74 | 48 | 21 | 3.2 |
| 936453 | 79 | 53 | 47 | 25 | 3.0 |
| 936456 | 77 | 57 | 49 | 32 | 3.9 |
| 936458 | 68 | 51 | 32 | 17 | 1.7 |

TABLE 46
Dose-dependent percent reduction of human
SPDEF RNA by modified oligonucleotides

| Compound | SPDEF (% UTC) | | | | |
|---|---|---|---|---|---|
| Number | 556 nM | 1667 nM | 5000 nM | 15000 nM | IC₅₀ μM |
| 833814 | 89 | 62 | 47 | 34 | 4.7 |
| 854302 | 64 | 34 | 33 | 20 | 1.1 |
| 936096 | 81 | 61 | 43 | 15 | 2.9 |
| 936097 | 90 | 77 | 44 | 37 | 5.7 |
| 936100 | 83 | 72 | 47 | 27 | 4.4 |
| 936102 | 95 | 83 | 61 | 33 | 7.5 |
| 936104 | 73 | 58 | 32 | 17 | 2.1 |
| 936106 | 69 | 61 | 45 | 34 | 3.6 |
| 936137 | 89 | 93 | 54 | 50 | 13.0 |
| 936139 | 104 | 84 | 53 | 62 | >15.0 |
| 936141 | 84 | 73 | 49 | 31 | 5.0 |
| 936169 | 65 | 49 | 30 | 33 | 1.7 |
| 936174 | 70 | 56 | 48 | 40 | 4.3 |
| 936179 | 97 | 86 | 53 | 32 | 6.6 |
| 936180 | 75 | 66 | 51 | 19 | 3.4 |
| 936184 | 74 | 63 | 44 | 25 | 3.2 |
| 936218 | 72 | 58 | 44 | 22 | 2.7 |
| 936256 | 90 | 76 | 55 | 36 | 6.8 |
| 936257 | 71 | 56 | 39 | 24 | 2.4 |

TABLE 47
Dose-dependent percent reduction of human
SPDEF RNA by modified oligonucleotides

| Compound | SPDEF (% UTC) | | | | |
|---|---|---|---|---|---|
| Number | 556 nM | 1667 nM | 5000 nM | 15000 nM | IC₅₀ μM |
| 833814 | 89 | 67 | 52 | 28 | 4.7 |
| 854302 | 55 | 35 | 21 | 18 | 0.6 |
| 936191 | 79 | 69 | 46 | 17 | 3.3 |
| 936192 | 77 | 62 | 38 | 45 | 4.7 |
| 936194 | 104 | 82 | 56 | 34 | 6.9 |
| 936198 | 66 | 60 | 41 | 28 | 2.6 |
| 936199 | 84 | 27 | 52 | 25 | 2.3 |
| 936208 | 75 | 64 | 44 | 33 | 3.9 |
| 936212 | 87 | 70 | 48 | 46 | 7.7 |
| 936214 | 68 | 36 | 40 | 21 | 1.4 |
| 936247 | 92 | 73 | 53 | 26 | 5.1 |
| 936249 | 84 | 62 | 56 | 35 | 5.5 |
| 936251 | 83 | 54 | 35 | 21 | 2.6 |
| 936252 | 61 | 38 | 30 | 22 | 1.0 |
| 936268 | 66 | 40 | 52 | 21 | 1.8 |
| 936273 | 107 | 82 | 58 | 32 | 6.9 |
| 936279 | 77 | 62 | 34 | 19 | 2.6 |
| 936284 | 74 | 82 | 47 | 30 | 4.9 |
| 936286 | 78 | 65 | 55 | 48 | 10.3 |

### Example 4: Tolerability of Modified Oligonucleotides Targeting Human SPDEF in CD-1 Mice

CD-1 mice are a multipurpose mouse model frequently utilized for safety and efficacy testing. The mice were treated with modified oligonucleotides selected from studies described above and evaluated for changes in the levels of various plasma chemistry markers.

### Study 1

Groups of four 6-to-8-week-old male CD-1 mice were injected subcutaneously once a week for six weeks (for a total of 6 treatments) with 50 mg/kg of modified oligonucleotides. One group of four male CD-1 mice was injected with saline. Mice were euthanized 72 hours following the final administration.

To evaluate the effect of modified oligonucleotides on liver and kidney function, plasma levels of aspartate aminotransferase (AST), alanine aminotransferase (ALT), total bilirubin (TBIL), blood urea nitrogen (BUN), creatinine (CRT) and albumin were measured using an automated clinical chemistry analyzer (Hitachi Olympus AU400c, Melville, N.Y.). The results are presented in the table below. Assays include four animals in a group, except where an asterisk (*) indicates that 3 animals or less was used for a specific assay. Modified oligonucleotides that caused changes in the levels of any of the liver or kidney function markers outside the expected range for modified oligonucleotides were excluded in further studies.

TABLE 49
Plasma chemistry markers in male CD-1 mice

| Compound | AST | ALT | TBIL | BUN | CRT | Albumin |
|---|---|---|---|---|---|---|
| No. | (IU/L) | (IU/L) | (mg/dL) | (mg/dL) | (mg/dL) | (mg/dL) |
| saline | 42 | 32 | 0.2 | 11* | 0.08 | 2.6 |
| 652522 | 1247 | 1720 | 0.2 | 14* | 0.09 | 2.6 |
| 801727 | 89 | 157 | 0.1 | 28 | 0.06 | 2.7 |
| 801919 | 103 | 102 | 0.2 | 26 | 0.10 | 2.6 |
| 801946 | 726 | 992 | 0.3 | 26 | 0.10 | 3.0 |
| 801965 | 238 | 376 | 0.2 | 24 | 0.06 | 2.7 |
| 802032 | 74 | 95 | 0.2 | 27 | 0.08 | 2.7 |
| 802094 | 738 | 999 | 0.2 | 24 | 0.08 | 2.6 |
| 802095 | 266 | 325 | 0.1 | 27 | 0.07 | 2.5 |

Body weights of CD-1 mice were measured at days 1 and 39, and the average body weight for each group is presented in the table below. Liver, kidney and spleen weights were measured at the end of the study and are presented in the table below. Modified oligonucleotides that caused any changes in organ weights outside the expected range for modified oligonucleotides were excluded from further studies.

TABLE 50
Body and organ weights (in grams)

| Compound | Body Weight (g) | | Liver | Kidney | Spleen |
|---|---|---|---|---|---|
| No. | Day 1 | Day 39 | (g) | (g) | (g) |
| saline | 32 | 39 | 2.0 | 0.5 | 0.1 |
| 652522 | 32 | 38 | 2.9 | 0.6 | 0.2 |
| 801727 | 33 | 39 | 2.6 | 0.6 | 0.1 |
| 801919 | 35 | 40 | 2.4 | 0.6 | 0.2 |
| 801946 | 33 | 35 | 1.7 | 0.6 | 0.1 |
| 801965 | 34 | 40 | 2.4 | 0.8 | 0.1 |
| 802032 | 34 | 38 | 2.2 | 0.6 | 0.1 |
| 802094 | 35 | 39 | 2.5 | 0.7 | 0.2 |
| 802095 | 35 | 42 | 2.9 | 0.7 | 0.2 |

### Study 2

Groups of 6-to-8-week-old male CD-1 mice were injected subcutaneously once a week for six weeks (for a total of 6 treatments) with 50 mg/kg of modified oligonucleotides. One group of male CD-1 mice was injected with PBS. Mice were euthanized 48 hours following the final administration.

To evaluate the effect of modified oligonucleotides on liver and kidney function, plasma levels of aspartate aminotransferase (AST), alanine aminotransferase (ALT), total bilirubin (TBIL), blood urea nitrogen (BUN), creatinine (CRT) and albumin were measured using an automated clinical chemistry analyzer (Hitachi Olympus AU400c, Melville, N.Y.). The results are presented in the table below. Modified oligonucleotides that caused changes in the levels of any of the liver or kidney function markers outside the expected range for modified oligonucleotides were excluded in further studies.

TABLE 51
Plasma chemistry markers in male CD-1 mice

| Compound | AST | ALT | TBIL | BUN | CRT | Albumin |
|---|---|---|---|---|---|---|
| No. | (IU/L) | (IU/L) | (mg/dL) | (mg/dL) | (mg/dL) | (mg/dL) |
| saline | 43 | 32 | 0.2 | 26 | 0.08 | 2.9 |
| 832911 | 174 | 412 | 0.1 | 36 | 0.11 | 3.1 |
| 833000 | 156 | 163 | 0.2 | 23 | 0.06 | 2.8 |
| 833013 | 649 | 1103 | 0.2 | 23 | 0.08 | 2.8 |
| 833188 | 2108 | 2158 | 0.3 | 22 | 0.09 | 3.3 |
| 833211 | 794 | 1629 | 1.1 | 22 | 0.08 | 2.8 |
| 833241 | 88 | 87 | 0.1 | 27 | 0.06 | 2.8 |
| 833243 | 95 | 57 | 0.1 | 24 | 0.06 | 2.7 |
| 833270 | 202 | 150 | 0.3 | 27 | 0.06 | 2.6 |
| 833343 | 539 | 527 | 0.4 | 23 | 0.05 | 2.7 |
| 833401 | 153 | 194 | 0.2 | 26 | 0.09 | 3.3 |
| 833413 | 235 | 357 | 0.2 | 21 | 0.04 | 2.5 |
| 833484 | 337 | 739 | 0.1 | 23 | 0.07 | 2.8 |
| 833486 | 87 | 111 | 0.1 | 21 | 0.04 | 2.6 |
| 833487 | 143 | 569 | 0.1 | 26 | 0.08 | 2.8 |
| 833488 | 198 | 353 | 0.1 | 18 | 0.05 | 2.9 |
| 833490 | 606 | 1042 | 0.2 | 18 | 0.10 | 3.6 |
| 833561 | 75 | 60 | 0.1 | 24 | 0.05 | 2.8 |
| 833580 | 89 | 125 | 0.1 | 21 | 0.09 | 2.9 |
| 833581 | 67 | 42 | 0.1 | 25 | 0.05 | 2.6 |
| 833631 | 108 | 77 | 0.1 | 21 | 0.04 | 2.7 |
| 833635 | 52 | 42 | 0.1 | 27 | 0.06 | 2.7 |
| 833678 | 445 | 392 | 0.4 | 19 | 0.04 | 2.6 |
| 833699 | 54 | 35 | 0.2 | 21 | 0.04 | 2.6 |
| 833711 | 98 | 117 | 0.1 | 22 | 0.03 | 2.5 |
| 833715 | 288 | 420 | 0.1 | 23 | 0.09 | 3.0 |
| 833733 | 514 | 545 | 0.1 | 19 | 0.04 | 2.6 |
| 833741 | 91 | 50 | 0.2 | 25 | 0.06 | 2.9 |

Body weights of CD-1 mice were measured at days 1 and 37, and the average body weight for each group is presented in the table below. Liver, kidney and spleen weights were measured at the end of the study and are presented in the table below. Modified oligonucleotides that caused any changes in organ weights outside the expected range for modified oligonucleotides were excluded from further studies.

TABLE 52
Body and organ weights (in grams)

| Compound | Body Weight (g) | | Liver | Kidney | Spleen |
|---|---|---|---|---|---|
| No. | Day 1 | Day 37 | (g) | (g) | (g) |
| saline | 34 | 43 | 2.3 | 0.6 | 0.1 |
| 832911 | 33 | 40 | 3.4 | 0.5 | 0.1 |
| 833000 | 34 | 41 | 2.8 | 0.6 | 0.1 |
| 833013 | 34 | 37 | 2.6 | 0.6 | 0.2 |
| 833188 | 33 | 40 | 4.1 | 0.7 | 0.3 |
| 833211 | 35 | 39 | 4.2 | 0.6 | 0.3 |
| 833241 | 33 | 38 | 2.2 | 0.5 | 0.1 |
| 833243 | 35 | 43 | 2.7 | 0.6 | 0.1 |
| 833270 | 34 | 43 | 3.3 | 0.6 | 0.5 |
| 833343 | 33 | 37 | 2.5 | 0.6 | 0.1 |
| 833401 | 35 | 43 | 2.7 | 0.6 | 0.1 |
| 833413 | 35 | 43 | 2.5 | 0.7 | 0.2 |
| 833484 | 33 | 42 | 3.3 | 0.6 | 0.2 |
| 833486 | 33 | 44 | 2.9 | 0.6 | 0.1 |
| 833487 | 34 | 43 | 3.2 | 0.6 | 0.1 |
| 833488 | 34 | 40 | 2.6 | 0.6 | 0.1 |
| 833490 | 33 | 39 | 3.3 | 0.6 | 0.2 |
| 833561 | 33 | 41 | 2.4 | 0.6 | 0.1 |
| 833580 | 35 | 41 | 2.7 | 0.6 | 0.1 |
| 833581 | 34 | 42 | 2.5 | 0.6 | 0.1 |
| 833631 | 32 | 41 | 2.5 | 0.6 | 0.2 |
| 833635 | 35 | 41 | 2.4 | 0.5 | 0.1 |
| 833678 | 33 | 39 | 2.6 | 0.6 | 0.2 |
| 833699 | 35 | 44 | 2.4 | 0.6 | 0.1 |
| 833711 | 35 | 41 | 2.7 | 0.6 | 0.2 |
| 833715 | 32 | 39 | 3.0 | 0.6 | 0.1 |
| 833733 | 34 | 40 | 2.9 | 0.6 | 0.2 |
| 833741 | 34 | 41 | 2.4 | 0.6 | 0.1 |

### Study 3

Groups of 6-to-8-week-old male CD-1 mice were injected subcutaneously once a week for six weeks (for a total of 6 treatments) with 50 mg/kg of modified oligonucleotides. One group of male CD-1 mice was injected with PBS. Mice were euthanized 48 hours following the final administration.

To evaluate the effect of modified oligonucleotides on liver and kidney function, plasma levels of aspartate aminotransferase (AST), alanine aminotransferase (ALT), total bilirubin (TBIL), blood urea nitrogen (BUN), creatinine (CRT) and albumin were measured using an automated clinical chemistry analyzer (Hitachi Olympus AU400c, Melville, N.Y.). The results are presented in the table below. Assays include four animals in a group, except where an asterisk (*) indicates that 3 animals or less was used for a specific assay. Modified oligonucleotides that caused changes in the levels of any of the liver or kidney function markers outside the expected range for modified oligonucleotides were excluded in further studies.

TABLE 53
Plasma chemistry markers in male CD-1 mice

| Compound | AST | ALT | TBIL | BUN | CRT | Albumin |
|---|---|---|---|---|---|---|
| No. | (IU/L) | (IU/L) | (mg/dL) | (mg/dL) | (mg/dL) | (mg/dL) |
| saline | 44 | 27 | 0.2 | 20 | 0.08 | 2.5 |
| 833748 | 94 | 64 | 0.2 | 20 | 0.08 | 2.4 |
| 833756 | 803 | 1338 | 0.3 | 19 | 0.08 | 2.9 |
| 833762 | 119 | 128 | 0.2 | 17 | 0.07 | 2.5 |
| 833767 | 95 | 129 | 0.2 | 22 | 0.07 | 2.5 |
| 833773 | 148 | 228 | 0.3 | 19 | 0.09 | 2.5 |
| 833813 | 71 | 43 | 0.2 | 19 | 0.07 | 2.4 |
| 833814 | 942 | 1598 | 0.2 | 17 | 0.08 | 2.4 |
| 833882 | 75 | 39 | 0.2 | 25 | 0.07 | 2.3 |
| 833886 | 167 | 142 | 0.2 | 21 | 0.08 | 2.5 |
| 833887 | 66 | 47 | 0.2 | 19 | 0.07 | 2.4 |
| 833904 | 110 | 94 | 0.2 | 18 | 0.07 | 2.5 |
| 833910 | 241 | 265 | 0.2 | 23 | 0.08 | 2.6 |
| 833951 | 252 | 425 | 0.2 | 13 | 0.05 | 2.3 |
| 833965 | 92 | 50 | 0.2 | 20 | 0.09 | 2.5 |
| 833973 | 129 | 94 | 0.2 | 18 | 0.07 | 2.4 |
| 854214 | 948 | 933 | 0.5 | 35 | 0.10 | 2.3 |
| 854254 | 296 | 471 | 0.2 | 19 | 0.07 | 2.6 |
| 854255 | 73 | 56 | 0.2 | 20 | 0.06 | 2.6 |
| 854302 | 255 | 283 | 0.2 | 23 | 0.11 | 2.5 |
| 854376 | 1485 | 2361 | 0.4 | 20 | 0.07 | 2.4 |
| 854459 | 100 | 104 | 0.2 | 21 | 0.06 | 2.4 |
| 854471* | 107 | 41 | 0.2 | 18 | 0.07 | 2.6 |
| 854527 | 1503 | 1976 | 0.4 | 17 | 0.07 | 2.5 |
| 854535 | 359 | 391 | 0.2 | 20 | 0.06 | 2 |
| 854537 | 358 | 475 | 0.2 | 20 | 0.09 | 2.5 |
| 854545 | 79 | 83 | 0.1 | 19 | 0.06 | 2.4 |

Body weights of CD-1 mice were measured at days 1 and 37, and the average body weight for each group is presented in the table below. Liver, kidney and spleen weights were measured at the end of the study and are presented in the table below. Modified oligonucleotides that caused any changes in organ weights outside the expected range for modified oligonucleotides were excluded from further studies.

TABLE 54
Body and organ weights (in grams)

| Compound | Body Weight (g) | | Liver | Kidney | Spleen |
|---|---|---|---|---|---|
| No. | Day 1 | Day 37 | (g) | (g) | (g) |
| saline | 32 | 39 | 1.9 | 0.5 | 0.1 |
| 833748 | 32 | 40 | 2.2 | 0.6 | 0.2 |
| 833756 | 31 | 37 | 2.7 | 0.5 | 0.1 |
| 833762 | 32 | 40 | 2.1 | 0.6 | 0.3 |
| 833767 | 31 | 40 | 2.3 | 0.6 | 0.2 |
| 833773 | 32 | 38 | 2.2 | 0.6 | 0.1 |
| 833813 | 32 | 38 | 2.0 | 0.6 | 0.2 |
| 833814 | 31 | 39 | 2.9 | 0.5 | 0.2 |
| 833882 | 32 | 40 | 2.1 | 0.6 | 0.2 |
| 833886 | 32 | 38 | 1.9 | 0.6 | 0.1 |
| 833887 | 32 | 38 | 2.0 | 0.6 | 0.1 |
| 833904 | 34 | 41 | 2.5 | 0.6 | 0.2 |
| 833910 | 32 | 38 | 2.0 | 0.6 | 0.1 |
| 833951 | 34 | 41 | 2.5 | 0.6 | 0.2 |
| 833965 | 32 | 40 | 2.2 | 0.6 | 0.1 |
| 833973 | 34 | 41 | 2.3 | 0.6 | 0.2 |
| 854214 | 33 | 38 | 1.5 | 0.5 | 0.1 |
| 854254 | 33 | 40 | 2.4 | 0.6 | 0.1 |
| 854255 | 32 | 39 | 2.2 | 0.6 | 0.1 |
| 854302 | 34 | 38 | 2.7 | 0.5 | 0.2 |
| 854376 | 33 | 35 | 2.4 | 0.5 | 0.1 |
| 854459 | 32 | 40 | 2.2 | 0.6 | 0.1 |
| 854471 | 35 | 42 | 2.2 | 0.6 | 0.2 |
| 854527 | 31 | 35 | 2.1 | 0.5 | 0.2 |
| 854535 | 33 | 40 | 2.7 | 0.6 | 0.3 |
| 854537 | 34 | 39 | 2.6 | 0.5 | 0.1 |
| 854545 | 33 | 40 | 2.1 | 0.6 | 0.2 |

### Study 4

Groups of 6-to-8-week-old male CD-1 mice were injected subcutaneously once a week for six weeks (for a total of 6 treatments) with 50 mg/kg of modified oligonucleotides. One group of male CD-1 mice was injected with PBS. Mice were euthanized 48 hours following the final administration.

To evaluate the effect of modified oligonucleotides on liver and kidney function, plasma levels of aspartate aminotransferase (AST), alanine aminotransferase (ALT), total bilirubin (TBIL), blood urea nitrogen (BUN), creatinine (CRT) and albumin were measured using an automated clinical chemistry analyzer (Hitachi Olympus AU400c, Melville, N.Y.). The results are presented in the table below. Assays include four animals in a group, except where an asterisk (*) indicates that 3 animals or less was used for a specific assay. Modified oligonucleotides that caused changes in the levels of any of the liver or kidney function markers outside the expected range for modified oligonucleotides were excluded in further studies.

TABLE 55
Plasma chemistry markers in male CD-1 mice

| Compound | AST | ALT | TBIL | BUN | CRT | Albumin |
|---|---|---|---|---|---|---|
| No. | (IU/L) | (IU/L) | (mg/dL) | (mg/dL) | (mg/dL) | (mg/dL) |
| saline | 48 | 32 | 0.2 | 22 | 0.08 | 2.6 |
| 854302 | 179 | 206 | 0.2 | 27 | 0.13 | 2.5 |
| 936069 | 90 | 77 | 0.2 | 24 | 0.10 | 2.8 |
| 936088 | 53 | 35 | 0.1 | 25 | 0.10 | 2.4 |
| 936096 | 61 | 48 | 0.2 | 23 | 0.09 | 2.6 |
| 936100 | 83 | 81 | 0.1 | 20 | 0.07 | 2.5 |
| 936104 | 51 | 44 | 0.2 | 19 | 0.10 | 2.8 |
| 936110 | 123 | 93 | 0.2 | 19 | 0.06 | 2.3 |
| 936142 | 64 | 45 | 0.2 | 23 | 0.08 | 2.5 |
| 936147 | 294 | 429 | 0.2 | 20 | 0.06 | 2.5 |
| 936158 | 96 | 90 | 0.2 | 21 | 0.06 | 2.4 |
| 936169* | 53 | 30 | 0.2 | 21 | 0.08 | 2.6 |
| 936198 | 67 | 46 | 0.1 | 21 | 0.06 | 2.5 |
| 936199 | 51 | 31 | 0.1 | 23 | 0.08 | 2.6 |
| 936208 | 125 | 106 | 0.2 | 27 | 0.08 | 2.5 |
| 936214 | 48 | 55 | 0.1 | 19 | 0.06 | 2.6 |
| 936218 | 43 | 38 | 0.2 | 18 | 0.07 | 2.7 |
| 936251 | 373 | 551 | 0.2 | 17 | 0.09 | 2.7 |
| 936268 | 85 | 60 | 0.1 | 18 | 0.06 | 2.5 |
| 936279 | 66 | 43 | 0.1 | 23 | 0.06 | 2.5 |
| 936315 | 314 | 457 | 0.1 | 18 | 0.03 | 2.5 |
| 936415 | 98 | 132 | 0.1 | 19 | 0.09 | 2.8 |

Body weights of CD-1 mice were measured at days 1 and 37, and the average body weight for each group is presented in the table below. Liver, kidney and spleen weights were measured at the end of the study and are presented in the table below. Modified oligonucleotides that caused any changes in organ weights outside the expected range for modified oligonucleotides were excluded from further studies.

TABLE 56
Body and organ weights (in grams)

| Compound | Body Weight (g) | | Liver | Kidney | Spleen |
|---|---|---|---|---|---|
| No. | Day 1 | Day 37 | (g) | (g) | (g) |
| saline | 29 | 35 | 1.9 | 0.5 | 0.1 |
| 854302 | 29 | 33 | 2.3 | 0.5 | 0.2 |
| 936069 | 29 | 35 | 2.2 | 0.5 | 0.1 |
| 936088 | 31 | 38 | 2.3 | 0.5 | 0.1 |
| 936096 | 31 | 37 | 2.1 | 0.6 | 0.1 |
| 936100 | 29 | 33 | 1.9 | 0.5 | 0.2 |
| 936104 | 29 | 38 | 2.0 | 0.6 | 0.1 |
| 936110 | 30 | 36 | 2.3 | 0.5 | 0.2 |
| 936142 | 29 | 35 | 1.9 | 0.5 | 0.1 |
| 936147 | 29 | 35 | 2.2 | 0.5 | 0.1 |
| 936158 | 29 | 35 | 2.0 | 0.5 | 0.2 |
| 936169* | 30 | 37 | 1.9 | 0.7 | 0.2 |
| 936198 | 29 | 37 | 2.0 | 0.6 | 0.1 |
| 936199 | 29 | 35 | 2.1 | 0.5 | 0.2 |
| 936208 | 29 | 35 | 2.2 | 0.5 | 0.1 |
| 936214 | 31 | 37 | 2.2 | 0.6 | 0.1 |
| 936218 | 29 | 36 | 2.0 | 0.6 | 0.2 |
| 936251 | 29 | 33 | 1.9 | 0.5 | 0.2 |
| 936268 | 31 | 39 | 2.2 | 0.6 | 0.2 |
| 936279 | 30 | 36 | 2.0 | 0.6 | 0.1 |
| 936315 | 29 | 37 | 2.6 | 0.5 | 0.2 |
| 936415 | 30 | 37 | 2.5 | 0.5 | 0.1 |

### Example 5: Local Tolerability of Modified Oligonucleotides Targeting Human SPDEF in CD-1 Mice

CD-1 mice are a multipurpose mouse model frequently utilized for safety and efficacy testing. The mice were treated with modified oligonucleotides selected from studies described above and evaluated for changes in the levels of various plasma chemistry markers.

### Study 1

Groups of 7-to-8-week-old male CD-1 mice were dosed orotracheally once a week for six weeks (for a total of 6 treatments) with 20 mg/kg of modified oligonucleotides. One group of male CD-1 mice was treated with saline. Mice were euthanized 48 hours following the final administration.

Body weights of CD-1 mice were measured at days 1 and 36, and the average body weight for each group is presented in the table below. Modified oligonucleotides that caused any changes in organ weights outside the expected range for modified oligonucleotides were excluded from further studies.

TABLE 57
Body weights (in grams)

| | |
|---|---|
| ION | Body Weight (g) |

| No. | Day 1 | Day 36 |
|---|---|---|
| Saline | 31 | 38 |
| 801727 | 33 | 41 |
| 801919 | 32 | 40 |
| 802032 | 33 | 40 |
| 833000 | 31 | 39 |
| 833241 | 32 | 38 |
| 833243 | 33 | 42 |
| 833401 | 30 | 36 |
| 833486 | 32 | 40 |
| 833561 | 31 | 37 |
| 833580 | 30 | 37 |
| 833581 | 31 | 39 |
| 833631 | 32 | 39 |
| 833635 | 32 | 40 |
| 833699 | 32 | 39 |
| 833711 | 31 | 40 |
| 833741 | 32 | 39 |

Bronchoalveolar Lavage (BAL) cellular profile

To evaluate the effect of modified oligonucleotides on lung function, levels of macrophages (MAC), neutrophils (NEU), lymphocytes (LYM), and eosinophils (EOS) in the bronchoalveolar lavage fluid (BAL) were measured. Mouse lungs were lavaged two times with 0.5 ml of PBS containing 1% BSA (Sigma-Aldrich). BAL fluid samples were centrifuged to generate a cell pellet and a cell-free supernatant. The recovered airway cells were resuspended in PBS with 1% BSA, and a cytospin was performed. Cells were stained with Diff-Quik stain (VWR). Data are presented as the percent of cells present in the total recovered BAL cell population.

Modified oligonucleotides that caused changes in the levels of any of the BAL markers outside the expected range for modified oligonucleotides were excluded in further studies. In some cases, where less than 4 samples were available in a group, the values are marked with an asterisk (*)

TABLE 58
Cellular profile in BAL

| Compound No. | MAC (%) | LYM (%) | EOS (%) | NEU (%) |
|---|---|---|---|---|
| saline | 97 | 2.3 | 0.0 | 0.8 |
| 801727 | 72 | 18.3 | 0.0 | 10.0 |
| 801919 | 87 | 4.8 | 0.3 | 8.3 |
| 802032 | 67* | 16.3* | 1.0* | 15.3* |
| 833000 | 71 | 14.8 | 0.8 | 16.5 |
| 833241 | 72* | 17.3* | 2.3* | 8.3* |
| 833243 | 76* | 10.3* | 3.3* | 10.3* |
| 833401 | 74 | 16.8 | 0.0 | 9.8 |
| 833486 | 64 | 17.0 | 0.0 | 19.0 |
| 833561 | 88* | 4.0* | 0.0* | 8.0* |
| 833580 | 94 | 3.5 | 0.0 | 2.8 |
| 833581 | 87 | 4.3 | 0.0 | 9.3 |
| 833631 | 95 | 5.8 | 0.0 | 4.8 |
| 833635 | 84 | 6.0 | 0.0 | 8.5 |
| 833699 | 85 | 9.5 | 1.5 | 2.5 |
| 833711 | 82 | 19.0 | 1.3 | 5.8 |
| 833741 | 88 | 7.5 | 1.0 | 3.3 |

### Bronchoalveolar Lavage (BAL) Cytokine Profile

To evaluate the effect of modified oligonucleotides on lung function, levels of Interleukin-10 (IL-10), Interleukin-6 (IL-6), monocyte chemotactic protein (MCP)-1/CCL2 and macrophage inflammatory protein (MIP)1β/CCL4 in the bronchoalveolar lavage fluid (BAL) were measured. BAL fluid was analyzed with MULTI_SPOT 96-well 4 spot prototype mouse 4-plex from MSD #N45ZA-1.

The results are presented in the table below. Modified oligonucleotides that caused changes in the levels of any of the BAL cytokines outside the expected range for modified oligonucleotides were excluded in further studies.

TABLE 60
Body weights (in grams)

| Compound | Body Weight (g) | |
|---|---|---|
| No. | Day 1 | Day 37 |
| saline | 31 | 39 |
| 833748 | 33 | 40 |
| 833762 | 33 | 39 |
| 833767 | 32 | 38 |
| 833813 | 31 | 37 |
| 833882 | 33 | 41 |
| 833886 | 31 | 36 |
| 833887 | 33 | 40 |
| 833904 | 33 | 38 |
| 833965 | 33 | 40 |
| 833973 | 31 | 37 |
| 854255 | 31 | 38 |
| 854459 | 32 | 38 |
| 854471 | 31 | 37 |
| 854545 | 32 | 39 |

### Bronchoalveolar Lavage (BAL) Cellular Profile

To evaluate the effect of modified oligonucleotides on lung function, levels of macrophages (MAC), neutrophils (NEU), lymphocytes (LYM), and eosinophils (EOS) in the bronchoalveolar lavage fluid (BAL) were measured. Mouse lungs were lavaged two times with 0.5 ml of PBS containing 1% BSA (Sigma-Aldrich). BAL fluid samples were centrifuged to generate a cell pellet and a cell-free supernatant. The recovered airway cells were resuspended in PBS with 1% BSA, and a cytospin was performed. Cells were stained with Diff-Quik stain (VWR). Data are presented as the percent of cells present in the total recovered BAL cell population.

Modified oligonucleotides that caused changes in the levels of any of the BAL markers outside the expected range for modified oligonucleotides were excluded in further studies. In some cases, where less than 4 samples were available in a group, the values are marked with an asterisk (*).

TABLE 61
Cellular profile in BAL

| Compound No. | MAC (%) | LYM (%) | EOS (%) | NEU (%) |
|---|---|---|---|---|
| Saline | 99 | 1.0 | 0.0 | 0.0 |
| 833748 | 93 | 2.3 | 0.5 | 3.8 |
| 833762 | 57 | 13.5 | 3.0 | 26.5 |
| 833767 | 62 | 21.3 | 0.0 | 17.0 |
| 833813 | 68 | 14.8 | 1.3 | 16.0 |
| 833882 | 70 | 24.3 | 0.5 | 5.5 |
| 833886 | 90 | 9.0 | 0.0 | 1.3 |
| 833887 | 87 | 8.8 | 0.0 | 4.3 |
| 833904 | 81 | 15.5 | 0.8 | 3.3 |
| 833965 | 88 | 8.3 | 0.8 | 3.0 |
| 833973 | 70 | 24.5 | 0.0 | 5.5 |
| 854255 | 77 | 15.5 | 0.0 | 7.8 |
| 854459 | 86 | 6.5 | 0.0 | 8.0 |
| 854471 | 72 | 16.3 | 1.0 | 10.8 |
| 854545 | 86* | 7.7* | 2.0* | 4.7* |

### Bronchoalveolar Lavage (BAL) Cytokine Profile

To evaluate the effect of modified oligonucleotides on lung function, levels of Interleukin-10 (IL-10), Interleukin-6 (IL-6), monocyte chemotactic protein (MCP)-1/CCL2 and macrophage inflammatory protein (MIP)1β/CCL4 in the bronchoalveolar lavage fluid (BAL) were measured. BAL fluid was analyzed with MULTI_SPOT 96-well 4 spot prototype mouse 4-plex from MSD.

The results are presented in the table below. Modified oligonucleotides that caused changes in the levels of any of the BAL cytokines outside the expected range for modified oligonucleotides were excluded in further studies.

TABLE 59
Cytokine profile in BAL

| Compound | IL-10 | IL-6 | CCL2 | CCL4 |
|---|---|---|---|---|
| Number | (pg/ml) | (pg/ml) | (pg/ml) | (pg/ml) |
| saline | 0.5 | 4.8 | 0 | 2 |
| 801727 | 0.5 | 10.3 | 104 | 387 |
| 801919 | 0.4 | 4.2 | 11 | 58 |
| 802032 | 0.4 | 4.7 | 106 | 245 |
| 833000 | 3.3 | 11.3 | 33 | 178 |
| 833241 | 1.6 | 53.8 | 439 | 1703 |
| 833243 | 0.9 | 6.0 | 33 | 159 |
| 833401 | 0.6 | 3.0 | 26 | 69 |
| 833486 | 1.1 | 5.9 | 363 | 2330 |
| 833561 | 0.4 | 4.9 | 43 | 107 |
| 833580 | 0.6 | 6.0 | 82 | 366 |
| 833581 | 0.2 | 3.2 | 4 | 36 |
| 833631 | 0.6 | 3.4 | 22 | 70 |
| 833635 | 0.5 | 2.6 | 10 | 69 |
| 833699 | 1.0 | 25.7 | 130 | 631 |
| 833711 | 1.7 | 2.9 | 7 | 25 |
| 833741 | 0.6 | 3.5 | 6 | 45 |

### Study 2

Groups of 7-to-8-week-old male CD-1 mice were dosed orotracheally once a week for six weeks (for a total of 6 treatments) with 20 mg/kg of modified oligonucleotides. One group of male CD-1 mice was treated with saline. Mice were euthanized 48 hours following the final administration.

Body weights of CD-1 mice were measured at days 1 and 37, and the average body weight for each group is presented in the table below. Modified oligonucleotides that caused any changes in organ weights outside the expected range for modified oligonucleotides were excluded from further studies.

TABLE 62
Cytokine profile in BAL

| Compound | IL-10 | IL-6 | CCL2 | CCL4 |
|---|---|---|---|---|
| No. | (pg/ml) | (pg/ml) | (pg/ml) | (pg/ml) |
| Saline | 0.5 | 0.9 | 1 | 20 |
| 833748 | 0.2 | 2.2 | 2 | 32 |
| 833762 | 2.0 | 26.4 | 3390 | 1195 |
| 833767 | 0.1 | 3.3 | 11 | 90 |
| 833813 | 5.6 | 31.8 | 760 | 2057 |
| 833882 | 0.7 | 9.8 | 111 | 297 |
| 833886 | 0.7 | 22.4 | 363 | 200 |
| 833887 | 0.5 | 4.8 | 141 | 270 |
| 833904 | 1.2 | 13.7 | 829 | 1919 |
| 833965 | 0.4 | 4.8 | 44 | 299 |
| 833973 | 3.5 | 17.3 | 195 | 1034 |
| 854255 | 0.9 | 9.5 | 397 | 426 |
| 854459 | 0.9 | 14.5 | 153 | 505 |
| 854471 | 4.3 | 53.1 | 987 | 3421 |
| 854545 | 0.2 | 2.9 | 59 | 108 |

### Study 3

Groups of 7-to-8-week-old male CD-1 mice were dosed orotracheally once a week for six weeks (for a total of 6 treatments) with 20 mg/kg of modified oligonucleotides. One group of male CD-1 mice was treated with saline. Mice were euthanized 72 hours following the final administration.

Body weights of CD-1 mice were measured at days 1 and 36, and the average body weight for each group is presented in the table below. Modified oligonucleotides that caused any changes in organ weights outside the expected range for modified oligonucleotides were excluded from further studies.

TABLE 63
Body weights (in grams)

| | |
|---|---|
| Compound | Body Weight (g) |

| No. | Day 1 | Day 36 |
|---|---|---|
| Saline | 28 | 35 |
| 854302 | 28 | 33 |
| 936069 | 27 | 34 |
| 936088 | 28 | 35 |
| 936096 | 28 | 33 |
| 936100 | 29 | 34 |
| 936104 | 28 | 35 |
| 936110 | 28 | 34 |
| 936142 | 28 | 36 |
| 936158 | 30 | 36 |

### Bronchoalveolar Lavage (BAL) Cellular Profile

To evaluate the effect of modified oligonucleotides on lung function, levels of macrophages (MAC), neutrophils (NEU), lymphocytes (LYM), and eosinophils (EOS) in the bronchoalveolar lavage fluid (BAL) were measured. Mouse lungs were lavaged two times with 0.5 ml of PBS containing 1% BSA (Sigma-Aldrich). BAL fluid samples were centrifuged to generate a cell pellet and a cell-free supernatant. The recovered airway cells were resuspended in PBS with 1% BSA, and a cytospin was performed. Cells were stained with Diff-Quik stain (VWR). Data are presented as the percent of cells present in the total recovered BAL cell population.

Modified oligonucleotides that caused changes in the levels of any of the BAL markers outside the expected range for modified oligonucleotides were excluded in further studies. In some cases, where less than 4 samples were available in a group, the values are marked with an asterisk (*).

TABLE 64
Cellular profile in BAL

| Compound No. | MAC (%) | LYM (%) | EOS (%) | NEU (%) |
|---|---|---|---|---|
| saline | 97 | 2.5 | 0.0 | 0.5 |
| 854302 | 97 | 2.0 | 0.0 | 1.3 |
| 936069 | 90 | 5.3 | 0.0 | 5.3 |
| 936088 | 79 | 2.8 | 0.0 | 18.0 |
| 936096 | 96 | 4.3 | 0.0 | 0.3 |
| 936100 | 69 | 16.0 | 0.0 | 15.5 |
| 936104 | 91 | 5.5 | 0.0 | 3.5 |
| 936110 | 85 | 12.8 | 0.0 | 2.5 |
| 936142 | 89 | 7.8 | 0.0 | 3.5 |
| 936158 | 96 | 2.0 | 0.0 | 2.0 |

### Bronchoalveolar Lavage (BAL) Cytokine Profile

To evaluate the effect of modified oligonucleotides on lung function, levels of Interleukin-10 (IL-10), Interleukin-6 (IL-6), monocyte chemotactic protein (MCP)-1/CCL2 and macrophage inflammatory protein (MIP)1β/CCL4 in the bronchoalveolar lavage fluid (BAL) were measured. BAL fluid was analyzed with MULTI_SPOT 96-well 4 spot prototype mouse 4-plex from MSD.

The results are presented in the table below. Modified oligonucleotides that caused changes in the levels of any of the BAL cytokines outside the expected range for modified oligonucleotides were excluded in further studies.

TABLE 65
Cytokine profile in BAL

| Compound | IL-10 | IL-6 | CCL2 | CCL4 |
|---|---|---|---|---|
| Number | (pg/ml) | (pg/ml) | (pg/ml) | (pg/ml) |
| Saline | N.D. | 0.4* | 1 | 21* |
| 854302 | 1.1* | 2.0 | 15 | 147 |
| 936069 | 0.6 | 0.5* | 12 | 79 |
| 936088 | 0.4 | 1.9 | 30 | 379 |
| 936096 | 1.2* | 1.2* | 28* | 71* |
| 936100 | 0.9 | 6.5 | 544 | 1078 |
| 936104 | 1.8 | 1.9* | 81 | 504 |
| 936110 | 0.5 | 1.2* | 15* | 75* |
| 936142 | 1.0* | 0.5* | 13* | 126* |
| 936158 | 0.4* | 0.9 | 3 | 72* |

### Study 4

Groups of 7-to-8-week-old male CD-1 mice were dosed orotracheally once a week for six weeks (for a total of 6 treatments) with 20 mg/kg of modified oligonucleotides. One group of male CD-1 mice was treated with saline. Mice were euthanized 72 hours following the final administration.

Body weights of CD-1 mice were measured at days 1 and 36, and the average body weight for each group is presented in the table below. Modified oligonucleotides that caused any changes in organ weights outside the expected range for modified oligonucleotides were excluded from further studies.

TABLE 66
Body weights (in grams)

| | |
|---|---|
| Compound | Body Weight (g) |

| No. | Day 1 | Day 36 |
|---|---|---|
| Saline | 32 | 39 |
| 936169 | 33 | 40 |
| 936198 | 32 | 41 |
| 936199 | 33 | 42 |
| 936208 | 33 | 42 |
| 936214 | 33 | 39 |
| 936218 | 34 | 40 |
| 936268 | 32 | 41 |
| 936279 | 32 | 39 |
| 936415 | 32 | 39 |

### Bronchoalveolar Lavage (BAL) Cellular Profile

To evaluate the effect of modified oligonucleotides on lung function, levels of macrophages (MAC), neutrophils (NEU), lymphocytes (LYM), and eosinophils (EOS) in the bronchoalveolar lavage fluid (BAL) were measured. Mouse lungs were lavaged two times with 0.5 ml of PBS containing 1% BSA (Sigma-Aldrich). BAL fluid samples were centrifuged to generate a cell pellet and a cell-free supernatant. The recovered airway cells were resuspended in PBS with 1% BSA, and a cytospin was performed. Cells were stained with Diff-Quik stain (VWR). Data are presented as the percent of cells present in the total recovered BAL cell population.

Modified oligonucleotides that caused changes in the levels of any of the BAL markers outside the expected range for modified oligonucleotides were excluded in further studies. In some cases, where less than 4 samples were available in a group, the values are marked with an asterisk (*).

TABLE 67
Cellular profile in BAL

| | |
|---|---|
| ION | BAL |

| No. | MAC (%) | LYM (%) | EOS (%) | NEU (%) |
|---|---|---|---|---|
| saline | 99 | 1.0 | 0.0 | 0.0 |
| 936169 | 89 | 6.3 | 0.0 | 5.5 |
| 936198 | 78 | 7.0 | 0.0 | 15.5 |
| 936199 | 59 | 25.3 | 0.0 | 16.3 |
| 936208 | 70 | 7.5 | 0.0 | 23.0 |
| 936214 | 71 | 3.3 | 0.0 | 26.3 |
| 936218 | 90 | 3.0 | 0.0 | 7.3 |
| 936268 | 91 | 4.8 | 0.0 | 4.5 |
| 936279 | 81 | 15.0 | 0.0 | 3.8 |
| 936415 | 43 | 9.5 | 0.8 | 46.5 |

### Bronchoalveolar Lavage (BAL) Cytokine Profile

To evaluate the effect of modified oligonucleotides on lung function, levels of Interleukin-10 (IL-10), Interleukin-6 (IL-6), monocyte chemotactic protein (MCP)-1/CCL2 and macrophage inflammatory protein (MIP)1β/CCL4 in the bronchoalveolar lavage fluid (BAL) were measured. BAL fluid was analyzed with MULTI_SPOT 96-well 4 spot prototype mouse 4-plex from MSD.

The results are presented in the table below. Modified oligonucleotides that caused changes in the levels of any of the BAL cytokines outside the expected range for modified oligonucleotides were excluded in further studies.

TABLE 68
Cytokine profile in BAL

Cytokines

| ION | IL-10 | IL-6 | CCL2 | CCL4 |
|---|---|---|---|---|
| No. | (pg/ml) | (pg/ml) | (pg/ml) | (pg/ml) |
| saline | 0.6* | 0.3* | 1* | 8* |
| 936169 | 0.5* | 1.4 | 20 | 141 |
| 936198 | 0.1* | 0.9 | 38 | 367 |
| 936199 | 14.8 | 21.6 | 660 | 2362 |
| 936208 | 0.8* | 0.8 | 25 | 212 |
| 936214 | 0.4* | 1.2 | 20 | 170 |
| 936218 | 0.7* | 9.8 | 33 | 170 |
| 936268 | 0.4* | 1.1* | 12 | 141 |
| 936279 | 1.0* | 3.3* | 38 | 203 |
| 936415 | 0.7 | 21.3 | 53 | 842 |

### Example 6: Activity of Modified Oligonucleotides Targeting Human SPDEF in Human Primary Bronchial Epithelial Cells (HBEs)

HBEs were obtained from Epithelix (Cat #EP61SA) and grown per manufacturer instructions.

### Study 1

HBEs were plated at 80,000 cells/well in a 96-well transwell plate, and an air-liquid interface (ALI) was established by differentiation for 5 weeks. Post establishment of ALI, the cells were treated with modified oligonucleotide at the concentrations indicated in the table below by free uptake at the basolateral surface. 72 hours post treatment, total RNA was isolated from the cells and SPDEF RNA levels were measured by quantitative real-time RTPCR. Human SPDEF primer probe set RTS35575 (forward sequence AAGTGCTCAAGGACATCGAG, designated herein as SEQ ID NO: 9; reverse sequence CGGTATTGGTGCTCTGTCC, designated herein as SEQ ID NO: 10; probe sequence TCCATGGGATCTGCGGTGATGTT, designated herein as SEQ ID NO: 11) was used to measure RNA levels as described above. SPDEF RNA levels were normalized to levels of cyclophilin A, measured by human primer probe set HTS3936 (forward sequence GCCATGGAGCGCTTTGG, designated herein as SEQ ID NO: 12; reverse sequence TCCACAGTCAGCAATGGTGATC, designated herein as SEQ ID NO: 13; probe sequence TCCAGGAATGGCAAGACCAGCAAGA, designated herein as SEQ ID NO: 14). Results are presented in the tables below as percent reduction of the amount of SPDEF RNA, relative to untreated control (UTC). The half maximal inhibitory concentration (IC₅₀) of each modified oligonucleotide is also presented. IC₅₀ was calculated using the log (inhibitor) vs response (three parameters) function in GraphPad Prism 7.01.

TABLE 69
Dose-dependent percent reduction of human SPDEF
RNA in HBEs by modified oligonucleotides

| Compound | SPDEF (% UTC) | | | |
|---|---|---|---|---|
| Number | 0.2 μM | 1 μM | 10 μM | IC₅₀ μM |
| 833561 | 21 | 10 | 0 | 0.06 |
| 833581 | 91 | 44 | 17 | 1.05 |
| 833631 | 40 | 13 | 3 | 0.14 |
| 833748 | 38 | 10 | 3 | 0.12 |
| 833886 | 27 | 6 | 1 | 0.07 |
| 936069 | 32 | 16 | 2 | 0.11 |
| 936096 | 17 | 4 | 1 | 0.04 |
| 936110 | 30 | 18 | 1 | 0.10 |
| 936218 | 53 | 8 | 1 | 0.19 |

### Study 2

HBEs were plated at 150,000 cells/well in a 24-well transwell plate, and an air-liquid interface (ALI) was established by differentiation for 5 weeks. Post establishment of ALI, the cells were treated with modified oligonucleotide at the concentrations indicated in the table below by free uptake at the basolateral surface. 72 hours post treatment, total RNA was isolated from the cells and SPDEF RNA levels were measured by quantitative real-time RTPCR. Human SPDEF primer probe set RTS35575 was used to measure RNA levels as described above. SPDEF RNA levels were normalized to cyclophilin A, as measured by human primer probe set HTS3936. Results are presented in the tables below as percent reduction of the amount of SPDEF RNA, relative to untreated control (UTC). The half maximal inhibitory concentration (IC₅₀) of each modified oligonucleotide is also presented. IC₅₀ was calculated using the log (inhibitor) vs response (three parameters) function in GraphPad Prism 7.01.

TABLE 70
Dose-dependent percent reduction of human SPDEF
RNA in HBEs by modified oligonucleotides

| Compound | SPDEF (% UTC) | | | | |
|---|---|---|---|---|---|
| Number | 0.01 μM | 0.1 μM | 1 μM | 10 μM | IC₅₀ μM |
| 833561 | 112 | 67 | 18 | 7 | 0.23 |
| 833741 | 102 | 78 | 31 | 12 | 0.44 |
| 833748 | 118 | 82 | 36 | 11 | 0.58 |
| 936110 | 151 | 110 | 26 | 10 | 0.68 |
| 936142 | 94 | 54 | 19 | 4 | 0.14 |
| 936158 | 89 | 78 | 39 | 13 | 0.58 |

In addition, RNA levels of airway secretory mucins MUC5AC and MUC5B were measured in the samples. SPDEF (sterile a-motif pointed domain epithelial specific transcription factor) is a known regulator of MUC5AC and MUC5B expression. Human MUC5AC primer probe set (ThermoFisher Scientific 4453320) and human MUC5B primer probe set (ThermoFisher Scientific 4448892) were used to measure MUC5A and MUC5B RNA levels as described above. RNA levels were normalized to cyclophilin A, as measured by human primer probe set HTS3936.

Knockdown of SPDEF led to a significant knockdown of MUC5AC as well as MUC5B RNA.

TABLE 71
Dose-dependent percent reduction of MUC5A/B RNA
in HBEs by modified oligonucleotides

| | MUC5AC | | | | | MUC5B | | | | |
|---|---|---|---|---|---|---|---|---|---|---|
| | (% UTC) | | | | | (% UTC) | | | | |
| Compound | 0.01 | 0.1 | 1 | 10 | IC₅₀ | 0.01 | 0.1 | 1 | 10 | IC₅₀ |
| Number | μM | μM | μM | μM | μM | μM | μM | μM | μM | μM |
| 833561 | 98 | 30 | 11 | 5 | 0.06 | 92 | 134 | 144 | 43 | 13.32 |
| 833741 | 56 | 42 | 17 | 4 | 0.18 | 85 | 87 | 51 | 54 | 4.50 |
| 833748 | 87 | 90 | 22 | 8 | 0.31 | 65 | 105 | 115 | 59 | 17.59 |
| 936110 | 119 | 60 | 20 | 6 | 0.15 | 68 | 93 | 94 | 60 | 14.60 |
| 936142 | 76 | 33 | 8 | 5 | 0.06 | 46 | 153 | 126 | 58 | 18.87 |
| 936158 | 58 | 47 | 21 | 6 | 0.06 | 99 | 98 | 99 | 77 | 33.54 |

### Study 3

HBEs were plated at 150,000 cells/well in a 24-well transwell plate, and an air-liquid interface (ALI) was established by differentiation for 5 weeks. Post establishment of ALI, the cells were treated with modified oligonucleotide at the concentrations indicated in the table below by free uptake at the basolateral surface. 72 hours post treatment, total RNA was isolated from the cells and SPDEF RNA levels were measured by quantitative real-time RTPCR. Human SPDEF primer probe set RTS35575 was used to measure RNA levels as described above. SPDEF RNA levels were normalized to cyclophilin A levels, as measured by human primer probe set HTS3936. Results are presented in the tables below as percent reduction of the amount of SPDEF RNA, relative to untreated control (UTC). The half maximal inhibitory concentration (IC₅₀) of each modified oligonucleotide is also presented. IC₅₀ was calculated using the log (inhibitor) vs response (three parameters) function in GraphPad Prism 7.01.

TABLE 72
Dose-dependent percent reduction of human SPDEF
RNA in HBEs by modified oligonucleotides

| Compound | SPDEF (% UTC) | | | |
|---|---|---|---|---|
| Number | 1 μM | 3 μM | 10 μM | IC₅₀ μM |
| 833741 | 55 | 24 | 17 | 1.20 |
| 833748 | 29 | 18 | 5 | 0.46 |
| 833965 | 58 | 42 | 27 | 1.99 |
| 854302 | 24 | 12 | 7 | 0.34 |
| 854459 | 55 | 33 | 16 | 1.38 |
| 854545 | 30 | 15 | 7 | 0.46 |
| 936142 | 37 | 21 | 8 | 0.66 |
| 936158 | 56 | 25 | 11 | 1.17 |

### Study 4

HBEs were plated at 500,000 cells/well in a 6 well transwell plate, and an air-liquid interface (ALI) was established by differentiation for 5 weeks. Post establishment of ALI, the cells were treated with modified oligonucleotide at the concentrations indicated in the table below by free uptake at the basolateral surface. 72 hours post treatment, total RNA was isolated from the cells and SPDEF RNA levels were measured by quantitative real-time RTPCR. Human SPDEF primer probe set RTS35575 was used to measure RNA levels as described above. SPDEF RNA levels were normalized to cyclophilin A levels, as measured by human primer probe set HTS3936. Results are presented in the tables below as percent reduction of the amount of SPDEF RNA, relative to untreated control (UTC).

In addition, RNA levels of airway secretory mucins MUCSAC and MUCSB were measured in the samples. Human MUCSAC primer probe set (ThermoFisher Scientific 4453320) and human MUCSB primer probe set (ThermoFisher Scientific 4448892) were used to measure MUCSAC and MUCSB RNA levels as described above. RNA levels were normalized to cyclophilin A, as measured by human primer probe set HTS3936. Knockdown of SPDEF led to significant knockdown of MUCSAC, as well as of MUCB RNA.

TABLE 73
Reduction of human SPDEF, MUC5AC, and MUC5B RNA
in HBEs by modified oligonucleotides

| | | |
|---|---|---|
| SPDEF | MUC5AC | MUC5B |
| (% UTC) | (% UTC) | (% UTC) |

| Compound | 2 | 10 | 2 | 10 | 2 | 10 |
|---|---|---|---|---|---|---|
| Number | μM | μM | μM | μM | μM | μM |
| 854302 | 4 | 0 | 31 | 13 | 45 | 37 |
| 936158 | 30 | 12 | 82 | 35 | 79 | 51 |

### Example 7: Tolerability of Modified Oligonucleotides Targeting Human SPDEF in Sprague-Dawley Rats

Sprague-Dawley rats are a multipurpose model used for safety and efficacy evaluations. The rats were treated with Ionis modified oligonucleotides from the studies described in the Examples above and evaluated for changes in the levels of various plasma chemistry markers.

### Study 1

Male Sprague-Dawley rats were maintained on a 12-hour light/dark cycle and fed ad libitum with Purina normal rat chow. Groups of 4 Sprague-Dawley rats each were weekly injected subcutaneously with 50 mg/kg of Ionis oligonucleotide for 6 weeks (total 6 doses). The rats were euthanized; and organs, urine and plasma were harvested for further analysis 3 days after the last dose.

### Plasma Chemistry Markers

To evaluate the effect of Ionis oligonucleotides on hepatic function, plasma levels of transaminases were measured using an automated clinical chemistry analyzer (Hitachi Olympus AU400c, Melville, N.Y.). Plasma levels of ALT (alanine transaminase) and AST (aspartate transaminase) were measured and the results are presented in the table below expressed in IU/L. Plasma levels of total bilirubin (TBIL), creatinine (CREA), albumin (ALB), and Blood Urea Nitrogen (BUN) were also measured using the same clinical chemistry analyzer and the results are also presented in the table below. Ionis modified oligonucleotides that caused changes in the levels of any markers of liver function outside the expected range for modified oligonucleotides were excluded in further studies. In some cases, where less than 4 samples were available in a group, the compounds are marked with an asterisk (*).

TABLE 74
Plasma chemistry markers in Sprague-Dawley rats

| Compound | ALT | AST | BUN | CREA | ALB | TBIL |
|---|---|---|---|---|---|---|
| No. | (IU/L) | (IU/L) | (mg/dL) | (mg/dL) | (g/dL) | (mg/dL) |
| Saline | 43 | 71 | 19 | 0.2 | 3.1 | 0.2 |
| 801919 | 52 | 83 | 36 | 0.3 | 2.0 | 0.2 |
| 833401 | 216 | 311 | 24 | 0.4 | 4.0 | 0.8 |
| 833561 | 43 | 70 | 22 | 0.3 | 3.6 | 0.2 |
| 833581 | 66 | 67 | 23 | 0.3 | 3.1 | 0.2 |
| 833631 | 76 | 133 | 23 | 0.3 | 2.6 | 0.3 |
| 833741* | 65 | 85 | 25 | 0.3 | 3.5 | 0.2 |
| 833748 | 37 | 55 | 20 | 0.3 | 3.3 | 0.2 |
| 833767 | 40 | 64 | 22 | 0.3 | 3.7 | 0.1 |
| 833886 | 41 | 81 | 34 | 0.5 | 3.2 | 0.1 |
| 833887 | 31 | 54 | 44 | 0.5 | 2.1 | 0.1 |
| 833965 | 44 | 79 | 20 | 0.3 | 3.3 | 0.2 |
| 854459 | 84 | 118 | 22 | 0.4 | 3.0 | 0.2 |
| 854545 | 34 | 76 | 18 | 0.3 | 3.2 | 0.2 |

Blood obtained from rat groups at week 6 were sent to IDEXX BioResearch for measurement of blood cell counts. Counts taken include red blood cell (RBC) count, white blood cell (WBC) count, hemoglobin (HGB), hematocrit (HCT), Mean corpuscular volume (MCV), mean corpuscular hemoglobin (MCH), mean corpuscular hemoglobin concentration (MCHC), and individual white blood cell counts, such as that of monocytes (MON), neutrophils (NEU), lymphocytes (LYM), and platelets (PLT). The results are presented in the tables below. Ionis oligonucleotides that caused changes in the blood cell count outside the expected range for modified oligonucleotides were excluded in further studies.

TABLE 75
Blood Cell Count in Sprague-Dawley Rats

| Compound | WBC | RBC | HGB | HCT | MCV | MCH | MCHC | NEU | LYM | MON | PLT |
|---|---|---|---|---|---|---|---|---|---|---|---|
| No. | (/nL) | (/pL) | (g/dL) | (%) | (fL) | (pg) | (%) | (%) | (%) | (%) | (/nL) |
| Saline | 10 | 8 | 15 | 43 | 53 | 18 | 35 | 12 | 81 | 5 | 892 |
| 801919 | 31 | 6 | 11 | 33 | 55 | 18 | 33 | 7 | 85 | 8 | 901 |
| 833401 | 14 | 8 | 13 | 37 | 47 | 17 | 36 | 17 | 74 | 8 | 1477 |
| 833561 | 9 | 9 | 15 | 43 | 49 | 18 | 36 | 10 | 84 | 6 | 853 |
| 833581 | 12 | 8 | 15 | 41 | 51 | 18 | 35 | 16 | 77 | 6 | 678 |
| 833631 | 14 | 8 | 14 | 40 | 50 | 17 | 35 | 12 | 78 | 10 | 969 |
| 833741* | 15 | 8 | 15 | 43 | 52 | 18 | 35 | 13 | 82 | 5 | 855 |
| 833748 | 11 | 8 | 15 | 42 | 50 | 18 | 35 | 8 | 88 | 4 | 869 |
| 833767 | 16 | 8 | 15 | 42 | 52 | 18 | 35 | 11 | 84 | 5 | 1043 |
| 833886 | 12 | 8 | 14 | 39 | 49 | 17 | 35 | 18 | 74 | 7 | 848 |
| 833887 | 18 | 8 | 13 | 39 | 51 | 17 | 34 | 9 | 87 | 4 | 787 |
| 833965 | 15 | 7 | 13 | 38 | 51 | 17 | 34 | 12 | 80 | 7 | 699 |
| 854459 | 10 | 8 | 13 | 37 | 48 | 17 | 36 | 4 | 88 | 7 | 594 |
| 854545 | 14 | 8 | 15 | 43 | 51 | 18 | 35 | 9 | 85 | 5 | 739 |

To evaluate the effect of Ionis oligonucleotides on kidney function, urinary levels of micro total protein (MTP) and creatinine were measured using an automated clinical chemistry analyzer (Hitachi Olympus AU400c, Melville, N.Y.). The ratios of MTP to creatinine (MTP/C ratio) are presented in the table below. Ionis oligonucleotides that caused changes in the levels of the ratio outside the expected range for modified oligonucleotides were excluded in further studies.

TABLE 76
MTP to creatinine ratio in Sprague-Dawley rats

| Compound No. | MTP/C Ratio |
|---|---|
| Saline | 1.8 |
| 801919 | 9.2 |
| 833401 | 7.1 |
| 833561 | 3.8 |
| 833581 | 5.0 |
| 833631 | 4.2 |
| 833741* | 4.9 |
| 833748 | 5.0 |
| 833767 | 3.4 |
| 833886 | 5.8 |
| 833887 | 11.1 |
| 833965 | 4.2 |
| 854459 | 1.5 |
| 854545 | 3.5 |

Body weights of rats were measured at days 1 and 40, and the average body weight for each group is presented in the table below. Liver, spleen and kidney weights were measured at the end of the study, and are presented in the table below. Ionis oligonucleotides that caused any changes in organ weights outside the expected range for modified oligonucleotides were excluded from further studies.

TABLE 77
Body and organ weights (g)

| Compound | Body Weight (g) | | Liver | Kidney | Spleen |
|---|---|---|---|---|---|
| No. | Day 1 | Day 40 | Weight (g) | Weight (g) | Weight (g) |
| Saline | 267 | 460 | 17 | 3.7 | 0.9 |
| 801919 | 248 | 310 | 16 | 3.9 | 2.3 |
| 833401 | 256 | 370 | 21 | 3.4 | 1.5 |
| 833561 | 256 | 360 | 13 | 3.1 | 1.1 |
| 833581 | 251 | 399 | 15 | 3.4 | 1.1 |
| 833631 | 253 | 397 | 16 | 3.4 | 1.3 |
| 833741 | 254 | 376 | 15 | 3.4 | 1.3 |
| 833748 | 261 | 412 | 16 | 3.2 | 1.2 |
| 833767 | 260 | 418 | 18 | 3.3 | 1.0 |
| 833886 | 256 | 371 | 20 | 3.3 | 1.8 |
| 833887 | 250 | 334 | 14 | 3.8 | 1.0 |
| 833965 | 253 | 427 | 17 | 3.3 | 1.3 |
| 854459 | 247 | 370 | 14 | 3.5 | 1.9 |
| 854545 | 254 | 394 | 14 | 3.2 | 1.1 |

### Study 2

Male Sprague-Dawley rats were maintained on a 12-hour light/dark cycle and fed ad libitum with Purina normal rat chow. Groups of 4 Sprague-Dawley rats each were weekly injected subcutaneously with 50 mg/kg of Ionis oligonucleotide for 6 weeks (total 6 doses). The rats were euthanized; and organs, urine and plasma were harvested for further analysis 3 days after the last dose.

### Plasma Chemistry Markers

To evaluate the effect of Ionis oligonucleotides on hepatic function, plasma levels of transaminases were measured using an automated clinical chemistry analyzer (Hitachi Olympus AU400c, Melville, N.Y.). Plasma levels of ALT (alanine transaminase) and AST (aspartate transaminase) were measured and the results are presented in the Table below expressed in IU/L. Plasma levels of total bilirubin (TBIL), creatinine (CREA), albumin (ALB), and Blood Urea Nitrogen (BUN) were also measured using the same clinical chemistry analyzer and the results are also presented in the Table below. Ionis modified oligonucleotides that caused changes in the levels of any markers of liver function outside the expected range for modified oligonucleotides were excluded in further studies.

TABLE 78
Plasma chemistry markers in Sprague-Dawley rats

| Compound | ALT | AST | BUN | ALB | CREA | TBIL |
|---|---|---|---|---|---|---|
| No. | (IU/L) | (IU/L) | (mg/dL) | (g/dL) | (mg/dL) | (mg/dL) |
| Saline | 39 | 59 | 17 | 3.2 | 0.2 | 0.2 |
| 854302 | 196 | 293 | 24 | 3.0 | 0.4 | 0.7 |
| 936069 | 1259 | 702 | 21 | 2.9 | 0.4 | 1.6 |
| 936096 | 38 | 70 | 22 | 3.2 | 0.4 | 0.2 |
| 936110 | 82 | 171 | 21 | 2.8 | 0.4 | 0.3 |
| 936142 | 36 | 69 | 19 | 3.2 | 0.3 | 0.1 |
| 936158 | 35 | 85 | 19 | 3.4 | 0.3 | 0.1 |
| 936169 | 42 | 70 | 19 | 3.1 | 0.3 | 0.1 |
| 936218 | 71 | 87 | 17 | 3.1 | 0.3 | 0.2 |
| 936268 | 34 | 67 | 17 | 3.4 | 0.3 | 0.1 |

Blood obtained from rat groups at week 6 were sent to IDEXX BioResearch for measurement of blood cell counts. Counts taken include red blood cell (RBC) count, white blood cell (WBC) count, hemoglobin (HGB), hematocrit (HCT), Mean corpuscular volume (MCV), mean corpuscular hemoglobin (MCH), mean corpuscular hemoglobin concentration (MCHC), and individual white blood cell counts, such as that of monocytes (MON), neutrophils (NEU), lymphocytes (LYM), and platelets (PLT). The results are presented in the tables below. Ionis oligonucleotides that caused changes in the blood cell count outside the expected range for modified oligonucleotides were excluded in further studies.

TABLE 79
Blood Cell Count in Sprague-Dawley Rats

| Compound | WBC | RBC | HGB | HCT | MCV | MCH | MCHC | NEU | LYM | MON | PLT |
|---|---|---|---|---|---|---|---|---|---|---|---|
| No. | (/nL) | (/pL) | (g/dL) | (%) | (fL) | (pg) | (%) | (%) | (%) | (%) | (/nL) |
| Saline | 9 | 8 | 15 | 45 | 55 | 19 | 34 | 13 | 85 | 2 | 908 |
| 854302 | 10 | 9 | 16 | 47 | 51 | 18 | 35 | 16 | 77 | 6 | 717 |
| 936069 | 14 | 9 | 15 | 46 | 53 | 18 | 33 | 12 | 81 | 6 | 872 |
| 936096 | 12 | 8 | 15 | 45 | 54 | 18 | 33 | 11 | 85 | 4 | 703 |
| 936110 | 13 | 7 | 13 | 40 | 54 | 18 | 34 | 9 | 87 | 3 | 606 |
| 936142 | 7 | 9 | 15 | 47 | 54 | 18 | 33 | 11 | 85 | 3 | 906 |
| 936158 | 7 | 8 | 15 | 44 | 54 | 18 | 34 | 11 | 85 | 3 | 763 |
| 936169 | 13 | 8 | 15 | 43 | 51 | 17 | 35 | 12 | 83 | 5 | 744 |
| 936218 | 10 | 8 | 15 | 45 | 53 | 18 | 34 | 10 | 87 | 3 | 787 |
| 936268 | 9 | 8 | 14 | 44 | 54 | 18 | 33 | 12 | 85 | 2 | 902 |

To evaluate the effect of Ionis oligonucleotides on kidney function, urinary levels of micro total protein (MTP) and creatinine were measured using an automated clinical chemistry analyzer (Hitachi Olympus AU400c, Melville, N.Y.). The ratios of MTP to creatinine (MTP/C ratio) are presented in the table below. Ionis oligonucleotides that caused changes in the levels of the ratio outside the expected range for modified oligonucleotides were excluded in further studies.

TABLE 80
MTP to creatinine ratio in Sprague-Dawley rats

| Compound | | | | | | | | | | |
|---|---|---|---|---|---|---|---|---|---|---|
| No. | saline | 854302 | 936069 | 936096 | 936110 | 936142 | 936158 | 936169 | 936218 | 936268 |
| MTP/C | 0.9 | 2.9 | 4.4 | 5.9 | 4.9 | 4.7 | 3.9 | 4.6 | 4.5 | 3.3 |
| Ratio | | | | | | | | | | |

Body weights of rats were measured at days 1 and 38, and the average body weight for each group is presented in the table below. Liver, spleen and kidney weights were measured at the end of the study, and are presented in the table below. Ionis oligonucleotides that caused any changes in organ weights outside the expected range for modified oligonucleotides were excluded from further studies.

TABLE 81
Body and organ weights (g)

| Compound | Body Weight (g) | | Liver | Kidney | Spleen |
|---|---|---|---|---|---|
| No. | Day 1 | Day 38 | Weight (g) | Weight (g) | Weight (g) |
| Saline | 301 | 458 | 16 | 3.7 | 0.7 |
| 854302 | 294 | 310 | 12 | 2.8 | 1.1 |
| 936069 | 292 | 321 | 14 | 3.5 | 1.3 |
| 936096 | 294 | 396 | 16 | 3.2 | 1.5 |
| 936110 | 295 | 376 | 14 | 3.5 | 1.9 |
| 936142 | 291 | 389 | 15 | 3.5 | 0.9 |
| 936158 | 291 | 424 | 17 | 3.6 | 1.1 |
| 936169 | 300 | 395 | 14 | 3.5 | 1.3 |
| 936218 | 285 | 328 | 12 | 3.0 | 1.0 |
| 936268 | 346 | 464 | 18 | 4.1 | 1.1 |

### Example 8: Effect of Modified Oligonucleotides Targeting Human SPDEF in Cynomolgus Monkeys, Inhalation Study

Cynomolgus monkeys were treated with Ionis modified oligonucleotides selected from studies described in the Examples above. Modified oligonucleotide tolerability was evaluated.

Prior to the study, the monkeys were housed according to Ionis-Specific NHP Socialization and Enrichment Guidelines (Laboratory Animal Science (Life Science) Work Instruction LAS 001). Six groups of 2 male and 2 female (a total of 4 animals) randomly assigned cynomolgus monkeys each were treated with aerosolized Ionis oligonucleotide or aerosolized saline by inhalation via face mask for 20-27 minutes. Following loading doses of 12 mg/kg on days 1, 3, 5, and 7, the monkeys were dosed once per week (on days 14, 21, 28, 35, and 42) with 12 mg/kg of Ionis oligonucleotide. Saline treated animals served as the control group.

During the study period, the monkeys were observed twice daily for signs of illness or distress. Any animal in poor health or in a possible moribund condition was identified for further monitoring and possible euthanasia. Animals were fasted overnight prior to necropsy. Scheduled euthanasia of the animals was conducted on day 43 approximately 24 hours after the last dose by exsanguination while under deep anesthesia. The protocols described in the Example were approved by the Institutional Animal Care and Use Committee (IACUC). The study complied with all applicable sections of the Final Rules of the Animal Welfare Act regulations (9 CFR Parts 1, 2, and 3) and the Guide for the Care and Use of Laboratory Animals National Research Council, National Academy Press Washington, D.C. Copyright 2011.

To evaluate the effect of Ionis oligonucleotides on the overall health of the animals, body and organ weights were measured. Terminal body weight was measured prior to necropsy. Organ weights were measured as well, and all weight measurements are presented in the table below. The results indicate that effect of treatment with modified oligonucleotides on body and organ weights was within the expected range for modified oligonucleotides.

TABLE 82
Body and Organ weights

| | Body | | | | | | | |
|---|---|---|---|---|---|---|---|---|
| | Weight | | | | | | | |
| | (kg) | | | | | | | |
| Compound | Day | Heart | Kidney | Liver | Lung | Spleen | Thymus | Brain |
| No. | 43 | (g) | (g) | (g) | (g) | (g) | (g) | (g) |
| Saline | 3.1 | 9 | 13 | 66 | 22 | 4 | 3 | 68 |
| 833561 | 3.0 | 8 | 12 | 56 | 19 | 2 | 3 | 66 |
| 833741 | 3.1 | 9 | 13 | 65 | 21 | 3 | 3 | 68 |
| 833748 | 2.9 | 9 | 12 | 55 | 20 | 3 | 2 | 67 |
| 936142 | 3.0 | 9 | 13 | 60 | 21 | 3 | 4 | 65 |
| 936158 | 3.0 | 9 | 14 | 69 | 23 | 4 | 2 | 69 |

To evaluate the effect of Ionis oligonucleotides on hepatic and kidney function, blood samples were collected from all the study groups on day 43. Whole blood was mixed with clot activator to allow clot formation for at least 30 minutes at room temperature. Serum was separated by centrifugation within 2 hours of collection. Levels of various liver function markers were measured using a Roche Cobas c501 Clinical Chemistry System (Roche Diagnostics, Indianapolis, Ind.). Blood urea nitrogen (BUN), creatinine (CREA), total protein (TP), albumin (ALB), alanine aminotransferase (ALT), aspartate aminotransferase (AST), total bilirubin (TBIL) were measured and the results are presented in the table below. The results indicate that modified oligonucleotides had no effect on liver and kidney function outside the expected range for modified oligonucleotides. Specifically, treatment with ION 833561 was well tolerated in terms of the liver and kidney function in monkeys.

TABLE 83
Liver and kidney function markers in cynomolgus monkey plasma

| Compound | BUN | CREA | TP | ALB | ALT | AST | TBIL |
|---|---|---|---|---|---|---|---|
| No. | (mg/dL) | (mg/dL) | (g/dL) | (g/dL) | (IU/L) | (IU/L) | (mg/dL) |
| Saline | 15 | 0.7 | 6.3 | 3.9 | 52 | 67 | 0.16 |
| 833561 | 13 | 0.9 | 6.7 | 4.1 | 46 | 78 | 0.16 |
| 833741 | 12 | 0.8 | 6.7 | 4.2 | 72 | 62 | 0.16 |
| 833748 | 12 | 1.0 | 6.8 | 4.3 | 35 | 58 | 0.16 |
| 936142 | 16 | 0.9 | 6.3 | 3.9 | 63 | 120 | 0.19 |
| 936158 | 14 | 0.9 | 6.6 | 4.1 | 54 | 141 | 0.18 |

To evaluate any inflammatory effect of Ionis oligonucleotides in cynomolgus monkeys, blood samples were taken for analysis. On day 42 (pre-dose) and day 43, approximately 1.0 mL of blood was collected from each animal and put into tubes with K₃EDTA for serum separation. The samples were centrifuged at 2,000 g for 10 min within an hour of collection. Complement C3 and Activated Factor B (Bb) were measured using a Beckman Image 800 analyzer and a Quidel Bb Plus ELISA kit respectively. The results indicate that treatment with ION 835561 did not cause any inflammation in monkeys. Another marker of inflammation, C-Reactive Protein (CRP) was tested together with the clinical chemistry parameters tested for liver function above.

TABLE 84
Pro-inflammatory protein analysis in cynomolgus monkeys

| | Complement | | Activated Factor | | |
|---|---|---|---|---|---|
| | C3 (mg/dL) | | B (Bb) (mg/dL) | | |
| | day 42 | day 43 | day 42 | day 43 | CRP |
| Compound | (pre- | (24 hr post- | (pre- | (24 hr post- | day 43 |
| No. | dose) | dose) | dose) | dose) | (mg/dL) |
| Saline | 92 | 84 | 1.1 | 1.4 | 11 |
| 833561 | 92 | 90 | 1.1 | 1.6 | 10 |
| 833741 | 83 | 85 | 1.3 | 2.0 | 4 |
| 833748 | 101 | 96 | 1.2 | 1.9 | 8 |
| 936142 | 95 | 85 | 1.2 | 2.6 | 15 |
| 936158 | 85 | 75 | 1.3 | 1.8 | 10 |

To evaluate any effect of Ionis oligonucleotides in cynomolgus monkeys on hematologic parameters, blood samples of approximately 0.5 mL of blood was collected from each of the available study animals on day 43. The samples were collected in tubes containing K₃EDTA. Samples were analyzed for red blood cell (RBC) count, Hemoglobin (HGB), Hematocrit (HCT), Mean corpuscular volume (MCV), mean corpuscular hemoglobin (MCH), mean corpuscular hemoglobin concentration (MCHC), platelet count (PLT), white blood cells (WBC) count, monocyte count (MON), neutrophil count (NEU), lymphocyte count (LYM), eosinophil count (EOS), and basophil count (BAS) using an ADVIA2120 hematology analyzer (Siemens, USA).

The data indicate the oligonucleotides did not cause any changes in hematologic parameters outside the expected range for modified oligonucleotides at this dose. Specifically, treatment with ION 833561 was well tolerated in terms of the hematologic parameters of the monkeys.

TABLE 85
Hematology analysis in cynomolgus monkeys

| Compound | RBC | HGB | HCT | MCV | MCH | MCHC |
|---|---|---|---|---|---|---|
| No. | (\hat{ }6/μL) | (g/dL) | (%) | (fL) | (pg) | (g/dL) |
| Saline | 5 | 13 | 42 | 78 | 24 | 31 |
| 833561 | 5 | 13 | 42 | 78 | 24 | 31 |
| 833741 | 6 | 14 | 45 | 80 | 24 | 30 |
| 833748 | 6 | 13 | 44 | 80 | 24 | 30 |
| 936142 | 5 | 13 | 41 | 81 | 25 | 31 |
| 936158 | 5 | 13 | 41 | 80 | 24 | 30 |

TABLE 86
Hematology analysis in cynomolgus monkeys

| Compound | WBC | NEU | LYM | MON | EOS | BAS | PLT |
|---|---|---|---|---|---|---|---|
| No. | (\hat{ }3/μL) | (\hat{ }3/μL) | (\hat{ }3/μL) | (\hat{ }3/μL) | (\hat{ }3/μL) | (\hat{ }3/μL) | (\hat{ }3/μL) |
| Saline | 11 | 5 | 5 | 0.51 | 0.08 | 0.04 | 450 |
| 833561 | 10 | 4 | 5 | 0.51 | 0.07 | 0.04 | 442 |
| 833741 | 10 | 4 | 5 | 0.35 | 0.03 | 0.03 | 521 |
| 833748 | 10 | 4 | 6 | 0.59 | 0.07 | 0.04 | 367 |
| 936142 | 14 | 7 | 6 | 0.82 | 0.08 | 0.05 | 419 |
| 936158 | 12 | 6 | 5 | 0.55 | 0.05 | 0.05 | 369 |

### Pharmacokinetic Analysis

Accumulation of modified oligonucleotides in various organs were measured in tissues collected at necropsy. Mean plasma concentrations at 24 hours post the last dose for all SPDEF modified oligonucleotides was evaluated. 833561 showed tissue and plasma accumulation profiles that were typical for this class of compound.

TABLE 87
Mean SPDEF modified oligonucleotide
Tissue Concentration (μg/g)

| | | Mean Concentration |
|---|---|---|
| Compound No. | Organ | (μg/g) |
| 833561 | Kidney | 196 |
| | Liver | 32 |
| | Lung | 186 |
| | Tracheal bronchial Lymph Node | 101 |
| | Prostate | ^{¶}8 |
| 833741 | Kidney | 180 |
| | Liver | 38 |
| | Lung | 412 |
| | Tracheal bronchial Lymph Node | 237 |
| | Prostate | ^{†}1 |
| 833748 | Kidney | 195 |
| | Liver | 38 |
| | Lung | 340 |
| | Tracheal bronchial Lymph Node | 213 |
| | Prostate | ^{¶}1 |
| 936142 | Kidney | 221 |
| | Liver | 42 |
| | Lung | 349 |
| | Tracheal bronchial Lymph Node | 248 |
| | Prostate | ^{¶}2 |
| 936158 | Kidney | 144 |
| | Liver | 25 |
| | Lung | 340 |
| | Tracheal bronchial Lymph Node | 171 |
| | Prostate | ^{†}1 |

^{¶}refers to groups with only 2 samples available
^{†}refers to groups with only 1 sample available

TABLE 88
Mean SPDEF modified oligonucleotide Plasma Concentration

| Compound No. | Mean Plasma Concentration (μg/ml) |
|---|---|
| 833561 | 0.1 |
| 833741 | 0.2 |
| 833748 | 0.1 |
| 936142 | 0.1 |
| 936158 | 0.1 |

### Bronchoalveolar Lavage (BAL) Cellular Analysis

Lung lavage was performed after collection of whole lung weight. The two washes were pooled and centrifuged at 300×g for 10 minutes. The pellet was resuspended in PBS in 1% BSA and a cytospin was performed. The slides were fixed and stained with modified Wright's stain (Siemens) with a Hematek 3000 instrument. The slides were used to obtain a cell differential using a Nikon E400 microscope. Cell counts taken include macrophages (MAC), neutrophils (NEU), eosinophils (EOS), and lymphocytes (LYM).

TABLE 89
Cellular profile in BAL

| Compound No. | MAC (%) | LYM (%) | EOS (%) | NEU (%) |
|---|---|---|---|---|
| Saline | 85 | 10 | 0 | 5 |
| 833561 | 89 | 7 | 0 | 4 |
| 833741 | 92 | 4 | 0 | 4 |
| 833748 | 85 | 12 | 0 | 3 |
| 936142 | 82 | 17 | 0 | 2 |
| 936158 | 86* | 14* | 0* | 1* |

*Samples available from only 2 animals

### Bronchoalveolar Lavage (BAL) Cytokine Profile

To evaluate the effect of modified oligonucleotides on lung function, levels of Interleukin-10 (IL-10), Interleukin-6 (IL-6), monocyte chemotactic protein (MCP)-1/CCL2, macrophage inflammatory protein (MIP)1β/CCL4, MIP-la, MCP-4, MDC and IP-10 in the bronchoalveolar lavage fluid (BAL) were measured. Cytokines were measured with 2 NHP kits from Meso Scale Diagnostics, LLC: U-PLEX Chemokine combo 1 K15055K-1 and U-PLEX TH17 Combo 1 K15079K-1.

The results are presented in the table below. Modified oligonucleotides that caused changes in the levels of any of the BAL cytokines outside the expected range for modified oligonucleotides were excluded in further studies. In some cases, the level of cytokine was too low to be measured accurately and is annotated as N/A.

TABLE 90
Cytokine profile in BAL

| Compound | IL-10 | IL-6 | MCP-1 | MIP-1β | MIP-1α | MCP-4 | MDC | IP-10 |
|---|---|---|---|---|---|---|---|---|
| No. | (pg/ml) | (pg/ml) | (pg/ml) | (pg/ml) | (pg/ml) | (pg/ml) | (pg/ml) | (pg/ml) |
| Saline | 0.01 | 0.5 | 370 | 4 | 20 | 6 | 114 | 73 |
| 833561 | N/A | 1.0 | 1314 | 7 | 28 | 8 | 193 | 872 |
| 833741 | N/A | 1.0 | 1475 | 6 | 33 | 7 | 307 | 137 |
| 833748 | 0.01 | 0.9 | 972 | 9 | 33 | 5 | 194 | 189 |
| 936142 | N/A | 0.8 | 1217 | 24 | 79 | 5 | 308 | 153 |
| 936158 | N/A | 0.5 | 2376 | 8 | 32 | 8 | 549 | 605 |

### Example 9: Effect of Modified Oligonucleotides on Cynomolgus Monkey SPDEF RNA In Vitro, Multiple Doses

Modified oligonucleotides selected from the examples above were tested at various doses in 4MBr-5 cells. Cultured 4MBr-5 cells at a density of 30,000 cells per well were treated with modified oligonucleotide at various doses by electroporation, as specified in the tables below. The electroporated cells were plated into culture media containing 50 ng/mL of human IL-13 protein (R&D systems #213-ILB-005). After an incubation of approximately 24 hours, total RNA was isolated from the cells and SPDEF RNA levels were measured by quantitative real-time RTPCR. Cynomolgus SPDEF primer probe set Mf02917915_ml was used to measure RNA levels as described above. SPDEF RNA levels were normalized to total RNA content, as measured by RIBOGREEN®. Results are presented in the tables below as percent reduction of the amount of SPDEF RNA, relative to untreated control (UTC). The half maximal inhibitory concentration (IC₅₀) of each modified oligonucleotide is also presented. IC₅₀ was calculated using a linear regression on a log/linear plot of the data in Excel.

TABLE 91
Dose-dependent percent reduction of cynomolgus monkey
SPDEF RNA by modified oligonucleotides

| | Number of | | | | | | |
|---|---|---|---|---|---|---|---|
| | Mismatches | SPDEF (% UTC) | | | | | |
| Compound | to Cyno | 20000 | 5000 | 1300 | 300 | 100 | IC₅₀ |
| Number | RNA | nM | nM | nM | nM | nM | μM |
| 833561 | 0 | 14 | 8 | 18 | 44 | 60 | 0.1 |
| 833741 | 1 | 6 | 21 | 50 | 58 | 77 | 0.7 |
| 833748 | 1 | 8 | 41 | 64 | 67 | 89 | 1.6 |
| 936158 | 2 | 25 | 56 | 72 | 74 | 75 | 4.3 |
| 936142 | 1 | 54 | 69 | 75 | 55 | 73 | >20 |

### Example 10: Effect of a Modified Oligonucleotide Complementary to SPDEF in a Bleomycin Induced Pulmonary Fibrosis Model

A group of twelve 12-week old male C57BL/6 mice (Jackson Laboratory) were treated with Compound No. 652553, and a group of twenty 12-week old male C57BL/6 mice (Jackson Laboratory) were similarly treated with control Compound No. 549148.

Both Compound Nos. 652553 and 549148 are 3-10-3 cEt gapmers, wherein they have a central gap segment of ten 2′-β-D-deoxynucleosides, wherein the 5′ and 3′ wing segments each consist of three cEt modified nucleosides, wherein the internucleoside linkages throughout the modified oligonucleotides are phosphorothioate (P═S) linkages, and wherein all cytosine nucleobases throughout the modified oligonucleotides are 5-methylcytosines. Compound No. 652553 has a sequence (from 5′ to 3′) of GCTCATGTGTATCCCT (SEQ ID NO: 2285), and is designed to be complementary to the mouse SPDEF target sequence, designated herein as SEQ ID NO: 2286 (GENBANK Accession No. NM_013891.4) at Start site 1540 and Stop site 1555, wherein “Start site” indicates the 5′-most nucleoside of the target sequence to which the modified oligonucleotide is complementary, and “Stop site” indicates the 3′-most nucleoside of the target sequence to which the modified oligonucleotide is complementary. Compound No. 549148 is a control oligonucleotide with a sequence (from 5′ to 3′) of GGCTACTACGCCGTCA (SEQ ID NO: 2287), and is designed to not target mouse SPDEF or any known gene.

Following a total of 3 loading doses of 10 mg/kg of modified oligonucleotide administered orotracheally twice per week prior to Day 0, the mice were dosed orotracheally twice per week with 10 mg/kg/dose of modified oligonucleotide for a total of 6 doses. Mice were sacrificed on Day 18 (48 hours post final dose of modified oligonucleotide). Following the loading dose, the mice were also treated with 2.5 u/kg of Bleomycin (Savmart, catalog #NDC-0783-3154-01) on Day 0 and 1.5u/kg of Bleomycin on Day 14. As a control, one group of twenty-four 12-week old male C57BL/6 mice (Jackson Laboratory) were treated with 2.5u/kg of Bleomycin on Day 0 and 1.5u/kg of Bleomycin on Day 14, without any treatment with modified oligonucleotide. The treatment groups were compared to a group of eight 12-week old male C57BL/6 mice (Jackson Laboratory) that were naïve and were not treated with either modified oligonucleotide or Bleomycin.

### Body Weights and Survivals

Body weights of C57BL/6 mice were measured, and the average body weight for each group om Day 0 and Day 18 are presented in the table below. In addition, the number of animals at the Days 0 and 18 were counted and are presented in the table below.

TABLE 92
Body weights (in grams) and survivals

| | Body weight (g) | | number of animals | |
|---|---|---|---|---|
| Treatment | Day 0 | Day 18 | Day 0 | Day 18 |
| naïve | 29 | 29 | 8 | 8 |
| Bleomycin alone | 28 | 26 | 24 | 21 |
| Belomycin + 549148 | 29 | 28 | 20 | 18 |
| Bleomycin + 652553 | 28 | 26 | 12 | 12 |

### Lung Function

Lung function was measured on Day 17 using the Penh score obtained through unrestrained plethysmography. A higher Penh score indicates more lung constriction. The results, shown in the table below, indicate that pre-treatment with a modified oligonucleotide complementary to SPDEF prevented the decrease in lung function (or the increase in Penh score) observed in the bleomycin induced pulmonary fibrosis mouse model.

TABLE 93
Penh Scores

| Treatment | Penh Score |
|---|---|
| naïve | 0.8 |
| Bleomycin alone | 5.4 |
| Bleomycin + 549148 | 3.1 |
| Belomycin + 652553 | 1.0 |

### RNA Analysis

On Day 18, RNA was extracted from the lungs of the mice for quantitative real-time RTPCR analysis of SPDEF RNA expression. In addition to SPDEF RNA levels, the RNA expression levels of various mouse lung fibrosis genes, including MUC5b, MUC5ac, COL1A1, ACTA2, TIMP1, and OPN, was tested using quantitative real-time RTPCR. The primer-probe sets used to measure levels of RNA of mouse SPDEF, MUC5b, MUC5ac, COL1A, ACTA2, TIMP1, and OPN are listed in the table below.

TABLE 94
List of mouse primer-probe sets used for RNA analysis

| | primer- | | SEQ | | SEQ | | SEQ |
|---|---|---|---|---|---|---|---|
| Target | probe set | Forward | ID | | ID | | ID |
| RNA | name | primer | NO. | Reverse Primer | NO. | Probe | NO. |
| SPDEF | RTS4444 | GCGAGGTC | 2288 | GCCACTTCTG | 2289 | CTTCTGAACAT | 2290 |
| | | CTGAAAGA | | CACGTTACCA | | CACAGCAGAC | |
| | | TATTGAG | | | | CCTGGG | |
| MUC5b | RTS3745 | TGACTCCA | 2291 | AGGTGTAAGG | 2292 | CACCTTCATCC | 2293 |
| | | TATCCTCA | | CGCTCATGCT | | CACCTATCACT | |
| | | TCCACAAG | | | | GTCTTCCC | |
| MUC5ac | RTS942 | TCACGTGC | 2294 | TGCTATCATC | 2295 | CCAGCCTTGTG | 2296 |
| | | CCTGATAA | | CCTGTAGCAG | | GCCCATCC | |
| | | CCAA | | TAGTG | | | |
| COL1A1 | mcolla1 | TGGATTCC | 2297 | TCAGCTGGAT | 2298 | AAGCGAGGGC | 2299 |
| | | CGTTCGAG | | AGCGACATC | | TCCGACCCGA | |
| | | TACG | | | | | |
| ACTA2 | mActa2_LTS00192 | TGCCTCTA | 2300 | GCAGGAATG | 2301 | CGTTTTGTGGA | 2302 |
| | | GCACACAA | | ATTTGGAAAG | | TCAGCGCCTCC | |
| | | CTGTGA | | GAA | | A | |
| TIMP1 | LTS00190 | TCATGGAA | 2303 | GCGGCCCGTG | 2304 | CCCACAAGTC | 2305 |
| | | AGCCTCTG | | ATGAGA | | CCAGAACCGC | |
| | | TGGAT | | | | AGTG | |
| OPN | RTS3534 | TGGTGCCT | | GTTTCTTGCTT | 2307 | AAGCAGAATC | 2308 |
| | | GACCCATC | 2306 | AAAGTCATCC | | TCCTTGCGCCA | |
| | | TCA | | TTTTCTT | | CAGAA | |

The levels of SPDEF RNA expression are presented as percent SPDEF RNA, relative to naïve control (% control). The levels of MUC5b RNA expression are presented as percent MUC5b RNA relative to naïve control (% control). The levels of MUC5ac RNA expression are presented as percent MUC5ac RNA relative to naïve control (% control). The levels of COL1A1 RNA expression are presented as percent COL1A1 RNA relative to naïve control (% control). The levels of ACTA2 RNA expression are presented as percent ACTA2 RNA relative to naïve control (% control). The levels of TIMP1 RNA expression are presented as percent TIMP1 RNA relative to naïve control (% control). The levels of OPN RNA expression are presented as percent OPN RNA relative to naïve control (% control).

As presented in the table below, treatment with SPDEF modified oligonucleotide resulted in reduction of SPDEF RNA in comparison to the naïve control. In addition, treatment with SPDEF modified oligonucleotide resulted in significant reduction of RNA expression of fibrosis markers compared to animals treated with bleomycin alone and compared to Bleomycin+549148.

TABLE 95
Modified oligonucleotide mediated inhibition of SPDEF
RNA expression and fibrosis gene RNA expression

| | | Bleomycin | Belomycin + | Bleomycin + |
|---|---|---|---|---|
| Gene | Naïve | alone | 549148 | 652553 |
| SPDEF | 100 | 160 | 158 | 90 |
| MUC5b | 100 | 159 | 173 | 79 |
| MUC5ac | 100 | 215 | 127 | 98 |
| COL1A1 | 100 | 2279 | 1398 | 918 |
| ACTA2 | 100 | 387 | 304 | 277 |
| TIMP1 | 100 | 6409 | 5102 | 2136 |
| OPN | 100 | 4279 | 4281 | 1341 |

### Example 11: Effect of a Modified Oligonucleotide Complementary to SPDEF in a Bleomycin Induced Pulmonary Fibrosis Model

Groups of twelve 12-week old male C57BL/6 mice (Jackson Laboratory) were treated with Compound No. 652553 (described herein above).

Following a total of 3 loading doses of 10 mg/kg/dose of Compound No. 652553 administered orotracheally twice per week prior to Day 0 (DO), the mice were dosed orotracheally twice per week with 10 mg/kg of modified oligonucleotide for a total of 9 doses. Following the loading dose, the mice were also treated with 2.5u/kg of Bleomycin on Day 0 and 2.5u/kg of Bleomycin on Day 7. One group of control mice were treated in a similar manner with saline instead of modified oligonucleotide. Mice were sacrificed on Day 21 (24 hours post final dose of modified oligonucleotide). The treatment groups were compared to a control group of twelve 12-week old male C57BL/6 mice (Jackson Laboratory) that were naïve and were not treated with either modified oligonucleotide or Bleomycin.

### Body Weights and Survivals

Body weights of CD-1 mice were measured at days 0 and 20, and the average body weight for each group is presented in the table below. In addition, the number of animals at the Days 0 and 20 were counted and are presented in the table below.

TABLE 96
Body weights (in grams) and survivals

| | Body weight (g) | | number of animals | |
|---|---|---|---|---|
| Treatment | Day 0 | Day 20 | Day 0 | Day 20 |
| naïve | 32 | 33 | 12 | 12 |
| Bleomycin + saline | 29 | 27 | 12 | 11 |
| Belomycin + 652553 | 29 | 29 | 12 | 12 |

### Lung Function

Lung function was measured on day 8 using the Penh score obtained through unrestrained plethysmography. A higher Penh score indicates more lung constriction. The results, shown in the table below, indicate that pre-treatment with a modified oligonucleotide complementary to SPDEF prevented the decrease in lung function (or increase in Penh score) observed in the bleomycin induced pulmonary fibrosis mouse model.

TABLE 97
Penh Scores

| Treatment | Penh Score |
|---|---|
| naïve | 0.9 |
| Belomycin + saline | 3.9 |
| Bleomycin + 652553 | 1.4 |

### RNA Analysis

On Day 21, RNA was extracted from the lungs of the mice for quantitative real-time RTPCR analysis of SPDEF RNA expression. In addition to SPDEF RNA levels, the RNA expression levels of various mouse lung fibrosis genes, including SPDEF, MUC5b, MUC5ac, COL1A1, ACTA2, TIMP1, CTGF, CHOP, GOB5, BiP and OPN was tested using quantitative real-time RTPCR. The primer-probe sets used to measure levels of RNA of mouse SPDEF, MUC5b, MUC5ac, COL1A1, ACTA2, TIMP1, CTGF, CHOP, GOB5 and OPN are listed in the table below. Additionally, IDT Technologies mouse primer probe set Mm.PT.58-6648074 was used to amplify FOXA3 RNA, IDT Technologies mouse primer probe set Mm.PT.58-43572495 was used to amplify AGR2 RNA, IDT technologies mouse primer probe set Mm.PT.58.6115287.g. was used to amplify BiP RNA, and IDT technologies mouse primer probe set 206445781 was used to amplify ATF4 RNA.

TABLE 98
List of mouse primer-probe sets used for RNA analysis

| | primer- | | SEQ | | SEQ | | SEQ |
|---|---|---|---|---|---|---|---|
| Target | probe set | Forward | ID | | ID | | ID |
| RNA | name | primer | NO. | Reverse Primer | NO. | Probe | NO. |
| SPDEF | RTS4444 | GCGAGGTC | 2288 | GCCACTTCTG | 2289 | CTTCTGAACATCAC | 2290 |
| | | CTGAAAGA | | CACGTTACCA | | AGCAGACCCTGGG | |
| | | TATTGAG | | | | | |
| MUC5b | RTS3745 | TGACTCCAT | 2291 | AGGTGTAAGG | 2292 | CACCTTCATCCCAC | 2293 |
| | | ATCCTCATC | | CGCTCATGCT | | CTATCACTGTCTTC | |
| | | CACAAG | | | | CC | |
| MUC5ac | RTS942 | TCACGTGC | 2294 | TGCTATCATC | 2295 | CCAGCCTTGTGGCC | 2296 |
| | | CCTGATAA | | CCTGTAGCAG | | CATCC | |
| | | CCAA | | TAGTG | | | |
| COL1Al | mcolla1 | TGGATTCCC | 2297 | TCAGCTGGAT | 2298 | AAGCGAGGGCTCC | 2299 |
| | | GTTCGAGT | | AGCGACATC | | GACCCGA | |
| | | ACG | | | | | |
| ACTA2 | mActa2_LTS00192 | TGCCTCTA | 2300 | GCAGGAATG | 2301 | CGTTTTGTGGATCA | 2302 |
| | | GCACACAA | | ATTTGGAAAG | | GCGCCTCCA | |
| | | CTGTGA | | GAA | | | |
| TIMP1 | LTS00190 | TCATGGAA | 2303 | GCGGCCCGTG | 2304 | CCCACAAGTCCCA | 2305 |
| | | AGCCTCTGT | | ATGAGA | | GAACCGCAGTG | |
| | | GGAT | | | | | |
| OPN | RTS3534 | TGGTGCCT | 2306 | GTTTCTTGCTT | 2307 | AAGCAGAATCTCC | 2308 |
| | | GACCCATC | | AAAGTCATCC | | TTGCGCCACAGAA | |
| | | TCA | | TTTTCTT | | | |
| CTGF | RTS352 | GCTCAGGG | 2309 | GCCCCCCACC | 2310 | TCATAATCAAAGA | 2311 |
| | | TAAGGTCC | | CCAAA | | AGCAGCAAGCACT | |
| | | GATTC | | | | TCCTG | |
| CHOP | mDDIT3_LTS00982 | TGAGCCTA | 2312 | TCTGGAACAC | 2313 | CAGCGACAGAGCC | 2314 |
| | | ACACGTCG | | TCTCTCCTCA | | AGAATAACAGCCG | |
| | | ATTATATCA | | GGTT | | | |
| Gob5 | RTS1845 | CACTAAGG | 2315 | AGCTCGCTTG | 2316 | CCCAGGCACGGCT | 2317 |
| | | TGGCCTAC | | AATGCTGTAT | | AAGGTTGGC | |
| | | CTCCAA | | TTC | | | |

The levels of SPDEF RNA expression are presented as percent SPDEF RNA, relative to bleomycin+saline treated animals, normalized to cyclophilin A (% control). Mouse cyclophilin A was amplified using primer probe set m_cyclo24 (forward sequence TCGCCGCTTGCTGCA, designated herein as SEQ ID NO: 2318; reverse sequence ATCGGCCGTGATGTCGA, designated herein as SEQ ID NO: 2319; probe sequence CCATGGTCAACCCCACCGTGTTC, designated herein as SEQ ID NO: 2320). The levels of MUC5b RNA expression are presented as percent MUC5b RNA relative to bleomycin+saline treated animals, normalized to cyclophilin A (% control). The levels of MUC5ac RNA expression are presented as percent MUC5ac RNA relative to bleomycin+saline treated animals, normalized to cyclophilin A (% control). The levels of COL1A1 RNA expression are presented as percent COL1A1 RNA relative to bleomycin+saline treated animals, normalized to cyclophilin A (% control). The levels of ACTA2 RNA expression are presented as percent ACTA2 RNA relative to bleomycin+saline treated animals, normalized to cyclophilin A (% control). The levels of TIMP1 RNA expression are presented as percent TIMP1 RNA relative to bleomycin+saline treated animals, normalized to cyclophilin A (% control). The levels of OPN RNA expression are presented as percent OPN RNA relative to bleomycin+saline treated animals, normalized to cyclophilin A (% control). The levels of CTGF RNA expression are presented as percent CTGF RNA relative to bleomycin+saline treated animals, normalized to cyclophilin A (% control). The levels of CHOP RNA expression are presented as percent CHOP RNA relative to bleomycin+saline treated animals, normalized to cyclophilin A (% control). The levels of BiP RNA expression are presented as percent BiP RNA relative to bleomycin+saline treated animals, normalized to cyclophilin A (% control). The levels of ATF4 RNA expression are presented as percent ATF4 RNA relative to bleomycin+saline treated animals, normalized to cyclophilin A (% control). The levels of Foxa3 RNA expression are presented as percent Foxa3 RNA relative to bleomycin+saline treated animals, normalized to cyclophilin A (% control). The levels of AGR2 RNA expression are presented as percent AGR2 RNA relative to bleomycin+saline treated animals, normalized to cyclophilin A (% control). The levels of GOB5 RNA expression are presented as percent GOB5 RNA relative to bleomycin+saline treated animals, normalized to cyclophilin A (% control).

As presented in the table below, treatment with SPDEF modified oligonucleotide resulted in reduction of SPDEF RNA in comparison to the naive and bleomycin+saline treated controls. In addition, treatment with SPDEF modified oligonucleotide resulted in significant reduction of RNA expression of mucous and fibrosis markers compared to animals treated with Bleomycin+saline.

TABLE 99
Modified oligonucleotide mediated inhibition of SPDEF
RNA expression and fibrosis gene RNA expression

| | | Bleomycin + | Belomycin + |
|---|---|---|---|
| Gene | Naïve | saline | 652553 |
| SPDEF (% control) | 198 | 100 | 60 |
| MUC5b (% control) | 244 | 100 | 41 |
| MUC5ac (% control) | 178 | 100 | 45 |
| COL1A1 (% control) | 37 | 100 | 50 |
| ACTA2 (% control) | 176 | 100 | 70 |
| TIMP1 (% control) | 13 | 100 | 45 |
| OPN (% control) | 11 | 100 | 26 |
| CTGF (% control) | 71 | 100 | 58 |
| CHOP (% control) | 157 | 100 | 93 |
| BiP (% control) | 154 | 100 | 86 |
| ATF4 (% control) | 152 | 100 | 76 |
| Foxa3 (% control) | 90 | 100 | 89 |
| Agr2 (% control) | 65 | 100 | 111 |
| Gob5 (% control) | 33 | 100 | 15 |

### Bronchoalveolar Lavage (BAL) Cellular Profile

To evaluate the effect of modified oligonucleotides on lung function, levels of macrophages (MAC), neutrophils (NEU), lymphocytes (LYM), and eosinophils (EOS) in the bronchoalveolar lavage fluid (BAL) were measured. Mouse lungs were lavaged two times with 0.5 ml of PBS containing 1% BSA (Sigma-Aldrich). BAL fluid samples were centrifuged at low speed to generate a cell pellet and a cell-free supernatant. The recovered airway cells were resuspended in PBS with 1% BSA, and a cytospin was performed. Cells were stained with Diff-Quik stain (VWR). Data are presented as the percent of cells present in the total recovered BAL cell population.

The results, shown in the table below, indicate that pre-treatment with a modified oligonucleotide complementary to SPDEF prevented the recruitment of inflammatory cells to the lungs.

TABLE 100
Cellular profile in BAL

| Treatment | MAC (%) | LYM (%) | EOS (%) | NEU (%) |
|---|---|---|---|---|
| naïve | 98 | 2 | 0 | 0 |
| Bleomycin + saline | 45 | 41 | 0 | 14 |
| Belomycin + 652553 | 77 | 11 | 0 | 12 |

### Example 12: Design of RNAi Compounds with Antisense RNAi Oligonucleotides Complementary to a Human SPDEF Nucleic Acid

RNAi compounds comprising antisense RNAi oligonucleotides complementary to a human SPDEF nucleic acid and sense RNAi oligonucleotides complementary to the antisense RNAi oligonucleotides were designed as follows.

The RNAi compounds in the tables below consist of an antisense RNAi oligonucleotide and a sense RNAi oligonucleotide, wherein, in each case the antisense RNAi oligonucleotides is 23 nucleosides in length; has a sugar motif (from 5′ to 3′) of: yfyfyfyfyfyfyfyfyfyfyyy; wherein “y” represents a 2′-O-methylribosyl sugar, and the “f” represents a 2′-fluororibosyl sugar; and a linkage motif (from 5′ to 3′) of: ssooooooooooooooooooss; wherein ‘o’ represents a phosphodiester internucleoside linkage and 's′ represents a phosphorothioate internucleoside linkage. The sense RNAi oligonucleotides in each case is 21 nucleosides in length; has a sugar motif (from 5′ to 3′) of: fyf yfyfyfyffyf, wherein “y” represents a 2′-O-methylribosyl sugar, and the “f” represents a 2′-fluororibosyl sugar; and a linkage motif (from 5′ to 3′) of: ssooooooooooooooooss; wherein ‘o’ represents a phosphodiester internucleoside linkage and 's′ represents a phosphorothioate internucleoside linkage. Each antisense RNAi oligonucleotide is complementary to the target nucleic acid (SPDEF), and each sense RNAi oligonucleotides is complementary to the first of the 21 nucleosides of the antisense RNAi oligonucleotide (from 5′ to 3′) wherein the last two 3′-nucleosides of the antisense RNAi oligonucleotides are not paired with the sense RNAi oligonucleotide (are overhanging nucleosides).

“Start site” indicates the 5′-most nucleoside to which the antisense RNAi oligonucleotides is complementary in the human gene sequence. “Stop site” indicates the 3′-most nucleoside to which the antisense RNAi oligonucleotide is complementary in the human gene sequence. Each modified antisense RNAi oligonucleoside listed in the tables below is 100% complementary to SEQ ID NO: 1 (described herein above).

TABLE 101
RNAi compounds targeting human SPDEF SEQ ID NO: 1

| | | | | SEQ ID | SEQ ID | | | |
|---|---|---|---|---|---|---|---|---|
| | | Antisense | SEQ | NO: 1 | NO: 1 | | | SEQ |
| Compound | Antisense | Sequence | ID | Antisense | Antisense | | Sense Sequence | ID |
| Number | ID | (5' to 3') | NO | Start Site | Stop Site | Sense ID | (5′ to 3′) | NO |
| 1527452 | 1527466 | GCAGGAAUGUGCU | 2324 | 25 | 47 | 1527461 | UUCCUCCCAGCA | 2511 |
| | | GGGAGGAAGU | | | | | CAUUCCUGC | |
| 1527453 | 1527469 | AGGGAGCUGGCAG | 2325 | 85 | 107 | 1527459 | CAAGCCUGCUGC | 2512 |
| | | CAGGCUUGGA | | | | | CAGCUCCCU | |
| 1527454 | 1527464 | CAGUGUGGACACG | 2326 | 45 | 67 | 1527458 | CACUCUGCCGUG | 2513 |
| | | GCAGAGUGCA | | | | | UCCACACUG | |
| 1527455 | 1527467 | AGUCAGACAGCCG | 2327 | 5 | 27 | 1527463 | UCAUCUCGCGGC | 2514 |
| | | CGAGAUGAAG | | | | | UGUCUGACU | |
| 1527456 | 1527468 | GGAGGACUGGGUC | 2328 | 65 | 87 | 1527460 | GCCCCACAGACC | 2515 |
| | | UGUGGGGCAG | | | | | CAGUCCUCC | |
| 1527457 | 1527465 | GCCCAACCUGAGG | 2329 | 105 | 127 | 1527462 | UGCAAGCCCCUC | 2516 |
| | | GGCUUGCAGG | | | | | AGGUUGGGC | |
| 1527470 | 1527486 | CCUGCUGGCACCG | 2330 | 125 | 147 | 1527479 | CCUUGCCACGGU | 2517 |
| | | UGGCAAGGCC | | | | | GCCAGCAGG | |
| 1527471 | 1527483 | CCCUCUGAGGUCU | 2331 | 185 | 207 | 1527476 | CAGCCCUGAGAC | 2518 |
| | | CAGGGCUGCG | | | | | CUCAGAGGG | |
| 1527473 | 1527487 | GCGUGCCUGUAGG | 2332 | 165 | 187 | 1527480 | GGGGACUCCCUA | 2519 |
| | | GAGUCCCCUA | | | | | CAGGCACGC | |
| 1527474 | 1527485 | CAGGUUGGCCACU | 2333 | 225 | 247 | 1527481 | AGGCCCCCAGUG | 2520 |
| | | GGGGGCCUGG | | | | | GCCAACCUG | |
| 1527475 | 1527482 | CUACCCCCAGCCC | 2334 | 145 | 167 | 1527478 | GCAGCCCUGGGC | 2521 |
| | | AGGGCUGCCU | | | | | UGGGGGUAG | |
| 1527488 | 1527502 | CUGGUGGCAGAGG | 2335 | 245 | 267 | 1527496 | GAGUGCUGCCUC | 2522 |
| | | CAGCACUCAG | | | | | UGCCACCAG | |
| 1527489 | 1527500 | GUGCCAACUUCAG | 2336 | 345 | 367 | 1527494 | CCCGGCCCCUGA | 2523 |
| | | GGGCCGGGAA | | | | | AGUUGGCAC | |
| 1527490 | 1527504 | GAAGAGGUGUGG | 2337 | 325 | 347 | 1527497 | CUCAGCUGCCCA | 2524 |
| | | GCAGCUGAGGC | | | | | CACCUCUUC | |
| 1527491 | 1527501 | CAGGCAUCUGGGG | 2338 | 285 | 307 | 1527495 | CGCUGGCCCCCC | 2525 |
| | | GGCCAGCGGA | | | | | AGAUGCCUG | |
| 1527492 | 1527505 | GGCCACUGGCGUG | 2339 | 305 | 327 | 1527499 | GGCUGAGACACG | 2526 |
| | | UCUCAGCCAG | | | | | CCAGUGGCC | |
| 1527493 | 1527503 | GGAACCAGGGGCC | 2340 | 265 | 287 | 1527498 | GCCCUGCUGGCC | 2527 |
| | | AGCAGGGCUG | | | | | CCUGGUUCC | |
| 1527506 | 1527518 | CCAGGGAGCUGUC | 2341 | 365 | 387 | 1527515 | CUGCAGCAGACA | 2528 |
| | | UGCUGCAGUG | | | | | GCUCCCUGG | |
| 1527507 | 1527522 | AGCAGGAGGUGGC | 2342 | 465 | 487 | 1527512 | AUCCCCCAGCCA | 2529 |
| | | UGGGGGAUAC | | | | | CCUCCUGCU | |
| 1527508 | 1527520 | UACGCUGCUCAGA | 2343 | 445 | 467 | 1527513 | AGCCCGGGUCUG | 2530 |
| | | CCCGGGCUGG | | | | | AGCAGCGUA | |
| 1527509 | 1527519 | GUCUGUUAGCUGC | 2344 | 385 | 407 | 1527514 | GGCACCAGGCAG | 2531 |
| | | CUGGUGCCCA | | | | | CUAACAGAC | |
| 1527510 | 1527523 | CUGUUUGGGCUGG | 2345 | 405 | 427 | 1527517 | CACAGCCGCCAG | 2532 |
| | | CGGCUGUGUC | | | | | CCCAAACAG | |
| 1527511 | 1527521 | UGGCGCUGCCCAU | 2346 | 425 | 447 | 1527516 | GCAGCGGCAUGG | 2533 |
| | | GCCGCUGCUG | | | | | GCAGCGCCA | |
| 1527524 | 1527537 | GCGACACCGUGUC | 2347 | 485 | 507 | 1527530 | UGCCCCCCGACA | 2534 |
| | | GGGGGGCAGC | | | | | CGGUGUCGC | |
| 1527525 | 1527536 | AAGGCGGACAGGC | 2348 | 585 | 607 | 1527532 | CGAGCAGGGCCU | 2535 |
| | | CCUGCUCGGG | | | | | GUCCGCCUU | |
| 1527526 | 1527539 | GGGCGUGGCGGGU | 2349 | 565 | 587 | 1527531 | CCCAGUCCACCC | 2536 |
| | | GGACUGGGAC | | | | | GCCACGCCC | |
| 1527527 | 1527538 | AGACCCACUGCCC | 2350 | 525 | 547 | 1527533 | GGCAGCGGGGGC | 2537 |
| | | CCGCUGCCGC | | | | | AGUGGGUCU | |
| 1527528 | 1527540 | GACUCCAGUCCCG | 2351 | 545 | 567 | 1527535 | UCGAGAGACGGG | 2538 |
| | | UCUCUCGAGA | | | | | ACUGGAGUC | |
| 1527529 | 1527541 | CGCCUUCUCCAAG | 2352 | 505 | 527 | 1527534 | CGGACAGGCUUG | 2539 |
| | | CCUGUCCGCG | | | | | GAGAAGGCG | |
| 1527542 | 1527556 | UGUCAAAGUAGG | 2353 | 605 | 627 | 1527549 | UCUACCUCUCCU | 2540 |
| | | AGAGGUAGAAG | | | | | ACUUUGACA | |
| 1527543 | 1527559 | CUGUCAAUGACCG | 2354 | 705 | 727 | 1527551 | GCAGUGCCCGGU | 2541 |
| | | GGCACUGCUC | | | | | CAUUGACAG | |
| 1527544 | 1527558 | CUCAGGCUCCUCA | 2355 | 685 | 707 | 1527550 | GAGCCACCUGAG | 2542 |
| | | GGUGGCUCCU | | | | | GAGCCUGAG | |
| 1527545 | 1527554 | CCUCCCGACUGCU | 2356 | 665 | 687 | 1527548 | CUGGGGCCAGCA | 2543 |
| | | GGCCCCAGGG | | | | | GUCGGGAGG | |
| 1527546 | 1527557 | GGGGCCUUGGCUG | 2357 | 645 | 667 | 1527552 | CAGCUGGGCAGC | 2544 |
| | | CCCAGCUGCU | | | | | CAAGGCCCC | |
| 1527547 | 1527555 | GCUGUCCUCAGGG | 2358 | 625 | 647 | 1527553 | AUGCUGUACCCU | 2545 |
| | | UACAGCAUGU | | | | | GAGGACAGC | |
| 1527560 | 1527572 | CCCGCCGGGCACC | 2359 | 745 | 767 | 1527567 | CUGGACUUGGUG | 2546 |
| | | AAGUCCAGGC | | | | | CCCGGCGGG | |
| 1527561 | 1527573 | GAGCACUUCGCCC | 2360 | 805 | 827 | 1527566 | AUGGUGGUGGGC | 2547 |
| | | ACCACCAUGG | | | | | GAAGUGCUC | |
| 1527562 | 1527574 | UGGACUGCACCUG | 2361 | 785 | 807 | 1527568 | CGCUGGAGCAGG | 2548 |
| | | CUCCAGCGAG | | | | | UGCAGUCCA | |
| 1527563 | 1527577 | GAGUGCUCCUCCA | 2362 | 765 | 787 | 1527571 | GCUGACCUUGGA | 2549 |
| | | AGGUCAGCCC | | | | | GGAGCACUC | |
| 1527564 | 1527575 | GGCUGCCCGCUGG | 2363 | 725 | 747 | 1527569 | GCCAAGCCCCAG | 2550 |
| | | GGCUUGGCUG | | | | | CGGGCAGCC | |
| 1527565 | 1527576 | CAGGCCGUCUCGA | 2364 | 825 | 847 | 1527570 | CAAGGACAUCGA | 2551 |
| | | UGUCCUUGAG | | | | | GACGGCCUG | |
| 1527578 | 1527590 | CGGUGAUGUUGA | 2365 | 845 | 867 | 1527587 | GCAAGCUGCUCA | 2552 |
| | | GCAGCUUGCAG | | | | | ACAUCACCG | |
| 1527579 | 1527591 | AGCUCCUGGAAGG | 2366 | 945 | 967 | 1527584 | GGGCAAGGCCUU | 2553 |
| | | CCUUGCCCAU | | | | | CCAGGAGCU | |
| 1527580 | 1527594 | CACUUCUGCACAU | 2367 | 885 | 907 | 1527585 | CCCCAGCAAUGU | 2554 |
| | | UGCUGGGGCU | | | | | GCAGAAGUG | |
| 1527581 | 1527595 | CAUGGGGGGCAGC | 2368 | 925 | 947 | 1527588 | CAAUACCGGCUG | 2555 |
| | | CGGUAUUGGU | | | | | CCCCCCAUG | |
| 1527582 | 1527592 | GGUGCUCUGUCCA | 2369 | 905 | 927 | 1527589 | GGCUCCUGUGGA | 2556 |
| | | CAGGAGCCAC | | | | | CAGAGCACC | |
| 1527583 | 1527593 | GCUCCAGUCCAUG | 2370 | 865 | 887 | 1527586 | GCAGAUCCCAUG | 2557 |
| | | GGAUCUGCGG | | | | | GACUGGAGC | |
| 1527596 | 1527598 | CGCACAGCUCCUU | 2371 | 965 | 987 | 1527597 | UGGCGGGCAAGG | 2558 |
| | | GCCCGCCAGC | | | | | AGCUGUGCG | |
| 1527599 | 1527610 | GAACUGCUCCUCC | 2372 | 985 | 1007 | 1527605 | GCCAUGUCGGAG | 2559 |
| | | GACAUGGCGC | | | | | GAGCAGUUC | |
| 1527600 | 1527609 | CCCAGGGGCGAGC | 2373 | 1005 | 1027 | 1527604 | CCGCCAGCGCUC | 2560 |
| | | GCUGGCGGAA | | | | | GCCCCUGGG | |
| 1527601 | 1527611 | UGACUUCCAGAUG | 2374 | 1045 | 1067 | 1527606 | CACCUGGACAUC | 2561 |
| | | UCCAGGUGGG | | | | | UGGAAGUCA | |
| 1527602 | 1527612 | GGGCGUGCAGCAC | 2375 | 1025 | 1047 | 1527607 | GUGGGGAUGUGC | 2562 |
| | | AUCCCCACCC | | | | | UGCACGCCC | |
| 1527603 | 1527613 | CGCUCUUUCAUCC | 2376 | 1065 | 1087 | 1527608 | AGCGGCCUGGAU | 2563 |
| | | AGGCCGCUGA | | | | | GAAAGAGCG | |
| 1527614 | 1527630 | CUGUCGGUCCAGC | 2377 | 1125 | 1147 | 1527625 | UGAGGAGAGCUG | 2564 |
| | | UCUCCUCACU | | | | | GACCGACAG | |
| 1527615 | 1527631 | ACUGGUCGAGGCA | 2378 | 1105 | 1127 | 1527622 | CACUACUGUGCC | 2565 |
| | | CAGUAGUGAA | | | | | UCGACCAGU | |
| 1527616 | 1527626 | AGCAUGAUGAGUC | 2379 | 1145 | 1167 | 1527621 | GCGAGGUGGACU | 2566 |
| | | CACCUCGCUG | | | | | CAUCAUGCU | |
| 1527617 | 1527627 | GAAUCGCCCCAGG | 2380 | 1085 | 1107 | 1527623 | GGACUUCACCUG | 2567 |
| | | UGAAGUCCGC | | | | | GGGCGAUUC | |
| 1527618 | 1527629 | CAGGUGGAUGGGC | 2381 | 1165 | 1187 | 1527620 | UCCGGGCAGCCC | 2568 |
| | | UGCCCGGAGC | | | | | AUCCACCUG | |
| 1527619 | 1527628 | AGCUGUGGGGCUU | 2382 | 1205 | 1227 | 1527624 | UGCUACUCAAGC | 2569 |
| | | GAGUAGCAAC | | | | | CCCACAGCU | |
| 1527632 | 1527649 | AUGCCCUUCUCCU | 2383 | 1245 | 1267 | 1527641 | GCUCAACAAGGA | 2570 |
| | | UGUUGAGCCA | | | | | GAAGGGCAU | |
| 1527633 | 1527647 | GGACGGUUCUUGC | 2384 | 1305 | 1327 | 1527638 | GGGCAUCCGCAA | 2571 |
| | | GGAUGCCCCA | | | | | GAACCGUCC | |
| 1527634 | 1527646 | CCACAGCCGGGCC | 2385 | 1285 | 1307 | 1527639 | GCCCAGGUGGCC | 2572 |
| | | ACCUGGGCUG | | | | | CGGCUGUGG | |
| 1527635 | 1527644 | CUGAGUCCUCAAU | 2386 | 1265 | 1287 | 1527642 | UCUUCAAAAUUG | 2573 |
| | | UUUGAAGAUG | | | | | AGGACUCAG | |
| 1527636 | 1527645 | AACUCCUUGAGGA | 2387 | 1185 | 1207 | 1527640 | GUGGCAGUUCCU | 2574 |
| | | ACUGCCACAG | | | | | CAAGGAGUU | |
| 1527637 | 1527648 | CCACCUAAUGAAG | 2388 | 1225 | 1247 | 1527643 | UAUGGCCGCUUC | 2575 |
| | | CGGCCAUAGC | | | | | AUUAGGUGG | |
| 1527650 | 1527663 | CUGGCGGAUGGAG | 2389 | 1345 | 1367 | 1527658 | CUGAGCCGCUCC | 2576 |
| | | CGGCUCAGCU | | | | | AUCCGCCAG | |
| 1527651 | 1527662 | AUGAUGCCCUUCU | 2390 | 1365 | 1387 | 1527659 | GUAUUACAAGAA | 2577 |
| | | UGUAAUACUG | | | | | GGGCAUCAU | |
| 1527652 | 1527665 | GGAGAGAGGCCCC | 2391 | 1465 | 1487 | 1527657 | CGCCCUCAGGGG | 2578 |
| | | UGAGGGCGGG | | | | | CCUCUCUCC | |
| 1527653 | 1527667 | GAACUGGUAGACG | 2392 | 1405 | 1427 | 1527656 | CAGCGCCUCGUC | 2579 |
| | | AGGCGCUGGG | | | | | UACCAGUUC | |
| 1527654 | 1527664 | GGGAGAUGUCUG | 2393 | 1385 | 1407 | 1527661 | UCCGGAAGCCAG | 2580 |
| | | GCUUCCGGAUG | | | | | ACAUCUCCC | |
| 1527655 | 1527666 | GCUUGUCGUAGUU | 2394 | 1325 | 1347 | 1527660 | CCGCCAUGAACU | 2581 |
| | | CAUGGCGGGA | | | | | ACGACAAGC | |
| 1527668 | 1527683 | GGGUUUCAGGCCC | 2395 | 1445 | 1467 | 1527679 | CUGGCCCAGGGC | 2582 |
| | | UGGGCCAGGC | | | | | CUGAAACCC | |
| 1527669 | 1527685 | UUGGGCUCUGGAA | 2396 | 1545 | 1567 | 1527676 | CUCUGACCUUCC | 2583 |
| | | GGUCAGAGCA | | | | | AGAGCCCAA | |
| 1527670 | 1527684 | GCAGCAGAGCAGA | 2397 | 1525 | 1547 | 1527678 | ACGGGCAGUCUG | 2584 |
| | | CUGCCCGUUU | | | | | CUCUGCUGC | |
| 1527671 | 1527682 | UGGCUGAGGCAGG | 2398 | 1485 | 1507 | 1527677 | CUGCCUGCCCUG | 2585 |
| | | GCAGGCAGGA | | | | | CCUCAGCCA | |
| 1527672 | 1527680 | UUUUCCCCCAUCU | 2399 | 1505 | 1527 | 1527674 | AGGCCCUGAGAU | 2586 |
| | | CAGGGCCUGG | | | | | GGGGGAAAA | |
| 1527673 | 1527681 | GGCACUCAGAUGG | 2400 | 1425 | 1447 | 1527675 | CGUGCACCCCAU | 2587 |
| | | GGUGCACGAA | | | | | CUGAGUGCC | |
| 1527686 | 1527699 | UUGGUUGCCCCUC | 2401 | 1565 | 1587 | 1527696 | AGGUCAGGGAGG | 2588 |
| | | CCUGACCUUG | | | | | GGCAACCAA | |
| 1527687 | 1527700 | GUCUCCCUCUGUC | 2402 | 1665 | 1687 | 1527694 | CCCUGGAGGACA | 2589 |
| | | CUCCAGGGGA | | | | | GAGGGAGAC | |
| 1527688 | 1527703 | GGAGCAGCUGGGC | 2403 | 1645 | 1667 | 1527693 | CUCCUCAGGCCC | 2590 |
| | | CUGAGGAGGA | | | | | AGCUGCUCC | |
| 1527689 | 1527702 | GGAAGCACCCCUG | 2404 | 1625 | 1647 | 1527695 | CCCUGGGGCAGG | 2591 |
| | | CCCCAGGGUC | | | | | GGUGCUUCC | |
| 1527690 | 1527701 | CCCAUAUCCCCCU | 2405 | 1585 | 1607 | 1527697 | ACUGCCCCAGGG | 2592 |
| | | GGGGCAGUUG | | | | | GGAUAUGGG | |
| 1527691 | 1527698 | GUCCCGAAGGCCC | 2406 | 1605 | 1627 | 1527692 | GUCCUCUGGGGC | 2593 |
| | | CAGAGGACCC | | | | | CUUCGGGAC | |
| 1527704 | 1527718 | AGGUGUUGGGGA | 2407 | 1685 | 1707 | 1527714 | CAGGGCUGCUCC | 2594 |
| | | GCAGCCCUGUC | | | | | CCAACACCU | |
| 1527705 | 1527721 | GGCAGGGGGAUG | 2408 | 1785 | 1807 | 1527710 | UCUCUGCUCCAU | 2595 |
| | | GAGCAGAGAGA | | | | | CCCCCUGCC | |
| 1527706 | 1527719 | GCCUUUGUCGAGU | 2409 | 1745 | 1767 | 1527712 | GGGCAGUGACUC | 2596 |
| | | CACUGCCCUU | | | | | GACAAAGGC | |
| 1527707 | 1527720 | AGAGGCCUGGACU | 2410 | 1765 | 1787 | 1527715 | CCACAGGCAGUC | 2597 |
| | | GCCUGUGGCC | | | | | CAGGCCUCU | |
| 1527708 | 1527716 | CUUCUGUAGGCUC | 2411 | 1725 | 1747 | 1527711 | CCAGAGCAGAGC | 2598 |
| | | UGCUCUGGAA | | | | | CUACAGAAG | |
| 1527709 | 1527717 | GAAAUGCUGGGG | 2412 | 1705 | 1727 | 1527713 | UGCCUCUGACCC | 2599 |
| | | UCAGAGGCAGG | | | | | CAGCAUUUC | |
| 1527722 | 1527734 | AUGUCUCCCUGCA | 2413 | 1825 | 1847 | 1527728 | UGGCAUGGUGCA | 2600 |
| | | CCAUGCCAGG | | | | | GGGAGACAU | |
| 1527723 | 1527736 | UCUAGUAUCUUUA | 2414 | 1885 | 1907 | 1527730 | AUGGAUAAUAA | 2601 |
| | | UUAUCCAUUC | | | | | AGAUACUAGA | |
| 1527724 | 1527732 | UGCCCAACUCAGG | 2415 | 1845 | 1867 | 1527731 | UCUGCACCCCUG | 2602 |
| | | GGUGCAGAUG | | | | | AGUUGGGCA | |
| 1527725 | 1527733 | UUCCCGGGGGCAC | 2416 | 1865 | 1887 | 1527727 | AGCCAGGAGUGC | 2603 |
| | | UCCUGGCUGC | | | | | CCCCGGGAA | |
| 1527726 | 1527735 | AGGUGUGGUGCA | 2417 | 1805 | 1827 | 1527729 | CUCCCAUUCUGC | 2604 |
| | | GAAUGGGAGGC | | | | | ACCACACCU | |
| 1528231 | 1528242 | GCCCUUCUUGUAA | 2418 | 1360 | 1382 | 1528234 | CGCCAGUAUUAC | 2605 |
| | | UACUGGCGGA | | | | | AAGAAGGGC | |
| 1528323 | 1528334 | UCCAGGCCGCUGA | 2419 | 1055 | 1077 | 1528331 | UCUGGAAGUCAG | 2606 |
| | | CUUCCAGAUG | | | | | CGGCCUGGA | |
| 1528361 | 1528371 | AAGCGGCCAUAGC | 2420 | 1215 | 1237 | 1528364 | GCCCCACAGCUA | 2607 |
| | | UGUGGGGCUU | | | | | UGGCCGCUU | |
| 1528397 | 1528406 | UCUUGUAAUACUG | 2421 | 1355 | 1377 | 1528400 | CCAUCCGCCAGU | 2608 |
| | | GCGGAUGGAG | | | | | AUUACAAGA | |
| 1537130 | 1537145 | GCUGGGAGGAAG | 2422 | 15 | 37 | 1537139 | GCUGUCUGACUU | 2609 |
| | | UCAGACAGCCG | | | | | CCUCCCAGC | |
| 1537131 | 1537141 | AGGGGCUUGCAGG | 2423 | 95 | 117 | 1537135 | GCCAGCUCCCUG | 2610 |
| | | GAGCUGGCAG | | | | | CAAGCCCCU | |
| 1537132 | 1537142 | GUCUGUGGGGCAG | 2424 | 55 | 77 | 1537136 | UGUCCACACUGC | 2611 |
| | | UGUGGACACG | | | | | CCCACAGAC | |
| 1537133 | 1537146 | CAGCAGGCUUGGA | 2425 | 75 | 97 | 1537137 | CCCAGUCCUCCA | 2612 |
| | | GGACUGGGUC | | | | | AGCCUGCUG | |
| 1537134 | 1537144 | CCGUGGCAAGGCC | 2426 | 115 | 137 | 1537138 | UCAGGUUGGGCC | 2613 |
| | | CAACCUGAGG | | | | | UUGCCACGG | |
| 1537140 | 1537147 | ACGGCAGAGUGCA | 2427 | 35 | 57 | 1537143 | CACAUUCCUGCA | 2614 |
| | | GGAAUGUGCU | | | | | CUCUGCCGU | |
| 1537148 | 1537160 | CCCAGGGCUGCCU | 2428 | 135 | 157 | 1537154 | GUGCCAGCAGGC | 2615 |
| | | GCUGGCACCG | | | | | AGCCCUGGG | |
| 1537149 | 1537161 | AGGCAGCACUCAG | 2429 | 235 | 257 | 1537155 | UGGCCAACCUGA | 2616 |
| | | GUUGGCCACU | | | | | GUGCUGCCU | |
| 1537151 | 1537162 | CAAGGGGUGGCCC | 2430 | 195 | 217 | 1537156 | ACCUCAGAGGGC | 2617 |
| | | UCUGAGGUCU | | | | | CACCCCUUG | |
| 1537152 | 1537163 | UCUCAGGGCUGCG | 2431 | 175 | 197 | 1537157 | UACAGGCACGCA | 2618 |
| | | UGCCUGUAGG | | | | | GCCCUGAGA | |
| 1537153 | 1537164 | AGGGAGUCCCCUA | 2432 | 155 | 177 | 1537159 | GCUGGGGGUAGG | 2619 |
| | | CCCCCAGCCC | | | | | GGACUCCCU | |
| 1537166 | 1537181 | GCCAGCAGGGCUG | 2433 | 255 | 277 | 1537174 | UCUGCCACCAGC | 2620 |
| | | GUGGCAGAGG | | | | | CCUGCUGGC | |
| 1537167 | 1537178 | GUCUGCUGCAGUG | 2434 | 355 | 377 | 1537173 | GAAGUUGGCACU | 2621 |
| | | CCAACUUCAG | | | | | GCAGCAGAC | |
| 1537168 | 1537183 | GGGCAGCUGAGGC | 2435 | 315 | 337 | 1537172 | CGCCAGUGGCCU | 2622 |
| | | CACUGGCGUG | | | | | CAGCUGCCC | |
| 1537169 | 1537179 | GUGUCUCAGCCAG | 2436 | 295 | 317 | 1537175 | CCAGAUGCCUGG | 2623 |
| | | GCAUCUGGGG | | | | | CUGAGACAC | |
| 1537170 | 1537182 | GGGGGCCAGCGGA | 2437 | 275 | 297 | 1537176 | CCCCUGGUUCCG | 2624 |
| | | ACCAGGGGCC | | | | | CUGGCCCCC | |
| 1537171 | 1537180 | CAGGGGCCGGGAA | 2438 | 335 | 357 | 1537177 | CACACCUCUUCC | 2625 |
| | | GAGGUGUGGG | | | | | CGGCCCCUG | |
| 1537184 | 1537199 | UGCCUGGUGCCCA | 2439 | 375 | 397 | 1537193 | CAGCUCCCUGGG | 2626 |
| | | GGGAGCUGUC | | | | | CACCAGGCA | |
| 1537185 | 1537197 | GGCUGGGGGAUAC | 2440 | 455 | 477 | 1537191 | UGAGCAGCGUAU | 2627 |
| | | GCUGCUCAGA | | | | | CCCCCAGCC | |
| 1537186 | 1537196 | AGACCCGGGCUGG | 2441 | 435 | 457 | 1537190 | GGGCAGCGCCAG | 2628 |
| | | CGCUGCCCAU | | | | | CCCGGGUCU | |
| 1537187 | 1537198 | CAUGCCGCUGCUG | 2442 | 415 | 437 | 1537192 | AGCCCAAACAGC | 2629 |
| | | UUUGGGCUGG | | | | | AGCGGCAUG | |
| 1537188 | 1537200 | UGGCGGCUGUGUC | 2443 | 395 | 417 | 1537194 | AGCUAACAGACA | 2630 |
| | | UGUUAGCUGC | | | | | CAGCCGCCA | |
| 1537189 | 1537201 | GUCGGGGGGCAGC | 2444 | 475 | 497 | 1537195 | CACCUCCUGCUG | 2631 |
| | | AGGAGGUGGC | | | | | CCCCCCGAC | |
| 1537202 | 1537216 | AAGCCUGUCCGCG | 2445 | 495 | 517 | 1537210 | CACGGUGUCGCG | 2632 |
| | | ACACCGUGUC | | | | | GACAGGCUU | |
| 1537204 | 1537218 | GGCCCUGCUCGGG | 2446 | 575 | 597 | 1537212 | CCGCCACGCCCG | 2633 |
| | | CGUGGCGGGU | | | | | AGCAGGGCC | |
| 1537205 | 1537219 | GGUGGACUGGGAC | 2447 | 555 | 577 | 1537211 | GGACUGGAGUCC | 2634 |
| | | UCCAGUCCCG | | | | | CAGUCCACC | |
| 1537206 | 1537217 | CCGUCUCUCGAGA | 2448 | 535 | 557 | 1537213 | GCAGUGGGUCUC | 2635 |
| | | CCCACUGCCC | | | | | GAGAGACGG | |
| 1537207 | 1537214 | CCCCCGCUGCCGC | 2449 | 515 | 537 | 1537208 | UGGAGAAGGCGG | 2636 |
| | | CUUCUCCAAG | | | | | CAGCGGGGG | |
| 1537220 | 1537233 | CUGCCCAGCUGCU | 2450 | 635 | 657 | 1537230 | CUGAGGACAGCA | 2637 |
| | | GUCCUCAGGG | | | | | GCUGGGCAG | |
| 1537221 | 1537235 | CCGGGCACUGCUC | 2451 | 695 | 717 | 1537227 | AGGAGCCUGAGC | 2638 |
| | | AGGCUCCUCA | | | | | AGUGCCCGG | |
| 1537222 | 1537237 | UGGGGCUUGGCUG | 2452 | 715 | 737 | 1537231 | GUCAUUGACAGC | 2639 |
| | | UCAAUGACCG | | | | | CAAGCCCCA | |
| 1537223 | 1537232 | UCAGGUGGCUCCU | 2453 | 675 | 697 | 1537228 | CAGUCGGGAGGA | 2640 |
| | | CCCGACUGCU | | | | | GCCACCUGA | |
| 1537224 | 1537236 | GCUGGCCCCAGGG | 2454 | 655 | 677 | 1537229 | GCCAAGGCCCCU | 2641 |
| | | GCCUUGGCUG | | | | | GGGGCCAGC | |
| 1537225 | 1537234 | GGGUACAGCAUGU | 2455 | 615 | 637 | 1537226 | CUACUUUGACAU | 2642 |
| | | CAAAGUAGGA | | | | | GCUGUACCC | |
| 1537238 | 1537250 | CCAAGGUCAGCCC | 2456 | 755 | 777 | 1537244 | UGCCCGGCGGGC | 2643 |
| | | GCCGGGCACC | | | | | UGACCUUGG | |
| 1537239 | 1537252 | CUGCUCCAGCGAG | 2457 | 775 | 797 | 1537248 | GAGGAGCACUCG | 2644 |
| | | UGCUCCUCCA | | | | | CUGGAGCAG | |
| 1537240 | 1537254 | ACCAAGUCCAGGC | 2458 | 735 | 757 | 1537249 | AGCGGGCAGCCU | 2645 |
| | | UGCCCGCUGG | | | | | GGACUUGGU | |
| 1537241 | 1537251 | CGAUGUCCUUGAG | 2459 | 815 | 837 | 1537245 | GCGAAGUGCUCA | 2646 |
| | | CACUUCGCCC | | | | | AGGACAUCG | |
| 1537242 | 1537255 | GAGCAGCUUGCAG | 2460 | 835 | 857 | 1537247 | GAGACGGCCUGC | 2647 |
| | | GCCGUCUCGA | | | | | AAGCUGCUC | |
| 1537243 | 1537253 | CCCACCACCAUGG | 2461 | 795 | 817 | 1537246 | GGUGCAGUCCAU | 2648 |
| | | ACUGCACCUG | | | | | GGUGGUGGG | |
| 1537256 | 1537268 | AUGGGAUCUGCGG | 2462 | 855 | 877 | 1537262 | CAACAUCACCGC | 2649 |
| | | UGAUGUUGAG | | | | | AGAUCCCAU | |
| 1537257 | 1537272 | CCACAGGAGCCAC | 2463 | 895 | 917 | 1537264 | GUGCAGAAGUGG | 2650 |
| | | UUCUGCACAU | | | | | CUCCUGUGG | |
| 1537258 | 1537269 | CAUUGCUGGGGCU | 2464 | 875 | 897 | 1537263 | UGGACUGGAGCC | 2651 |
| | | CCAGUCCAUG | | | | | CCAGCAAUG | |
| 1537259 | 1537270 | CUUGCCCGCCAGC | 2465 | 955 | 977 | 1537267 | UUCCAGGAGCUG | 2652 |
| | | UCCUGGAAGG | | | | | GCGGGCAAG | |
| 1537260 | 1537271 | AGGCCUUGCCCAU | 2466 | 935 | 957 | 1537265 | UGCCCCCCAUGG | 2653 |
| | | GGGGGGCAGC | | | | | GCAAGGCCU | |
| 1537261 | 1537273 | AGCCGGUAUUGGU | 2467 | 915 | 937 | 1537266 | GACAGAGCACCA | 2654 |
| | | GCUCUGUCCA | | | | | AUACCGGCU | |
| 1537274 | 1537285 | CACAUCCCCACCC | 2468 | 1015 | 1037 | 1537280 | UCGCCCCUGGGU | 2655 |
| | | AGGGGCGAGC | | | | | GGGGAUGUG | |
| 1537275 | 1537282 | AUGUCCAGGUGGG | 2469 | 1035 | 1057 | 1537278 | GCUGCACGCCCA | 2656 |
| | | CGUGCAGCAC | | | | | CCUGGACAU | |
| 1537276 | 1537284 | AGCGCUGGCGGAA | 2470 | 995 | 1017 | 1537279 | AGGAGCAGUUCC | 2657 |
| | | CUGCUCCUCC | | | | | GCCAGCGCU | |
| 1537277 | 1537283 | UCCGACAUGGCGC | 2471 | 975 | 997 | 1537281 | GGAGCUGUGCGC | 2658 |
| | | ACAGCUCCUU | | | | | CAUGUCGGA | |
| 1537287 | 1537291 | AGGUGAAGUCCGC | 2472 | 1075 | 1097 | 1537289 | AUGAAAGAGCGG | 2659 |
| | | UCUUUCAUCC | | | | | ACUUCACCU | |
| 1537292 | 1537304 | GCACAGUAGUGAA | 2473 | 1095 | 1117 | 1537299 | UGGGGCGAUUCA | 2660 |
| | | UCGCCCCAGG | | | | | CUACUGUGC | |
| 1537293 | 1537306 | GGAACUGCCACAG | 2474 | 1175 | 1197 | 1537300 | CCAUCCACCUGU | 2661 |
| | | GUGGAUGGGC | | | | | GGCAGUUCC | |
| 1537294 | 1537305 | CUUGAGUAGCAAC | 2475 | 1195 | 1217 | 1537298 | CUCAAGGAGUUG | 2662 |
| | | UCCUUGAGGA | | | | | CUACUCAAG | |
| 1537295 | 1537308 | GUCCACCUCGCUG | 2476 | 1135 | 1157 | 1537301 | UGGACCGACAGC | 2663 |
| | | UCGGUCCAGC | | | | | GAGGUGGAC | |
| 1537296 | 1537307 | AGCUCUCCUCACU | 2477 | 1115 | 1137 | 1537303 | CCUCGACCAGUG | 2664 |
| | | GGUCGAGGCA | | | | | AGGAGAGCU | |
| 1537297 | 1537309 | GGCUGCCCGGAGC | 2478 | 1155 | 1177 | 1537302 | CUCAUCAUGCUC | 2665 |
| | | AUGAUGAGUC | | | | | CGGGCAGCC | |
| 1537311 | 1537325 | UGCGGAUGCCCCA | 2479 | 1295 | 1317 | 1537316 | CCCGGCUGUGGG | 2666 |
| | | CAGCCGGGCC | | | | | GCAUCCGCA | |
| 1537312 | 1537324 | GUUCAUGGCGGGA | 2480 | 1315 | 1337 | 1537319 | AAGAACCGUCCC | 2667 |
| | | CGGUUCUUGC | | | | | GCCAUGAAC | |
| 1537313 | 1537327 | AAUUUUGAAGAU | 2481 | 1255 | 1277 | 1537317 | GAGAAGGGCAUC | 2668 |
| | | GCCCUUCUCCU | | | | | UUCAAAAUU | |
| 1537314 | 1537322 | GCCACCUGGGCUG | 2482 | 1275 | 1297 | 1537320 | UGAGGACUCAGC | 2669 |
| | | AGUCCUCAAU | | | | | CCAGGUGGC | |
| 1537315 | 1537326 | CCUUGUUGAGCCA | 2483 | 1235 | 1257 | 1537321 | UCAUUAGGUGGC | 2670 |
| | | CCUAAUGAAG | | | | | UCAACAAGG | |
| 1537329 | 1537340 | UGGGGUGCACGAA | 2484 | 1415 | 1437 | 1537334 | UCUACCAGUUCG | 2671 |
| | | CUGGUAGACG | | | | | UGCACCCCA | |
| 1537330 | 1537342 | ACGAGGCGCUGGG | 2485 | 1395 | 1417 | 1537336 | AGACAUCUCCCA | 2672 |
| | | AGAUGUCUGG | | | | | GCGCCUCGU | |
| 1537331 | 1537343 | UGGCUUCCGGAUG | 2486 | 1375 | 1397 | 1537337 | AAGGGCAUCAUC | 2673 |
| | | AUGCCCUUCU | | | | | CGGAAGCCA | |
| 1537332 | 1537345 | GAGCGGCUCAGCU | 2487 | 1335 | 1357 | 1537338 | CUACGACAAGCU | 2674 |
| | | UGUCGUAGUU | | | | | GAGCCGCUC | |
| 1537333 | 1537344 | CCCUGGGCCAGGC | 2488 | 1435 | 1457 | 1537339 | AUCUGAGUGCCU | 2675 |
| | | ACUCAGAUGG | | | | | GGCCCAGGG | |
| 1537346 | 1537359 | CCCUGAGGGCGGG | 2489 | 1455 | 1477 | 1537354 | GCCUGAAACCCG | 2676 |
| | | UUUCAGGCCC | | | | | CCCUCAGGG | |
| 1537347 | 1537358 | CUCCCUGACCUUG | 2490 | 1555 | 1577 | 1537352 | CCAGAGCCCAAG | 2677 |
| | | GGCUCUGGAA | | | | | GUCAGGGAG | |
| 1537348 | 1537361 | GAAGGUCAGAGCA | 2491 | 1535 | 1557 | 1537355 | UGCUCUGCUGCU | 2678 |
| | | GCAGAGCAGA | | | | | CUGACCUUC | |
| 1537349 | 1537363 | AGACUGCCCGUUU | 2492 | 1515 | 1537 | 1537357 | AUGGGGGAAAAC | 2679 |
| | | UCCCCCAUCU | | | | | GGGCAGUCU | |
| 1537350 | 1537360 | UCUCAGGGCCUGG | 2493 | 1495 | 1517 | 1537353 | UGCCUCAGCCAG | 2680 |
| | | CUGAGGCAGG | | | | | GCCCUGAGA | |
| 1537351 | 1537362 | AGGGCAGGCAGGA | 2494 | 1475 | 1497 | 1537356 | GGCCUCUCUCCU | 2681 |
| | | GAGAGGCCCC | | | | | GCCUGCCCU | |
| 1537364 | 1537378 | GGCCUGAGGAGGA | 2495 | 1635 | 1657 | 1537374 | GGGGUGCUUCCU | 2682 |
| | | AGCACCCCUG | | | | | CCUCAGGCC | |
| 1537365 | 1537376 | CCCCAGAGGACCC | 2496 | 1595 | 1617 | 1537371 | GGGGAUAUGGG | 2683 |
| | | AUAUCCCCCU | | | | | UCCUCUGGGG | |
| 1537366 | 1537377 | GAGCAGCCCUGUC | 2497 | 1675 | 1697 | 1537372 | CAGAGGGAGACA | 2684 |
| | | UCCCUCUGUC | | | | | GGGCUGCUC | |
| 1537367 | 1537381 | GUCCUCCAGGGGA | 2498 | 1655 | 1677 | 1537370 | CCAGCUGCUCCC | 2685 |
| | | GCAGCUGGGC | | | | | CUGGAGGAC | |
| 1537368 | 1537379 | CCUGGGGCAGUUG | 2499 | 1575 | 1597 | 1537375 | GGGGCAACCAAC | 2686 |
| | | GUUGCCCCUC | | | | | UGCCCCAGG | |
| 1537369 | 1537380 | CUGCCCCAGGGUC | 2500 | 1615 | 1637 | 1537373 | GCCUUCGGGACC | 2687 |
| | | CCGAAGGCCC | | | | | CUGGGGCAG | |
| 1537382 | 1537397 | GGUCAGAGGCAGG | 2501 | 1695 | 1717 | 1537389 | CCCCAACACCUG | 2688 |
| | | UGUUGGGGAG | | | | | CCUCUGACC | |
| 1537383 | 1537394 | CAGAAUGGGAGGC | 2502 | 1795 | 1817 | 1537393 | AUCCCCCUGCCU | 2689 |
| | | AGGGGGAUGG | | | | | CCCAUUCUG | |
| 1537384 | 1537398 | UGGAGCAGAGAG | 2503 | 1775 | 1797 | 1537390 | UCCAGGCCUCUC | 2690 |
| | | AGGCCUGGACU | | | | | UCUGCUCCA | |
| 1537385 | 1537396 | ACUGCCUGUGGCC | 2504 | 1755 | 1777 | 1537388 | UCGACAAAGGCC | 2691 |
| | | UUUGUCGAGU | | | | | ACAGGCAGU | |
| 1537386 | 1537399 | AGUCACUGCCCUU | 2505 | 1735 | 1757 | 1537391 | GCCUACAGAAGG | 2692 |
| | | CUGUAGGCUC | | | | | GCAGUGACU | |
| 1537387 | 1537395 | CUCUGCUCUGGAA | 2506 | 1715 | 1737 | 1537392 | CCCAGCAUUUCC | 2693 |
| | | AUGCUGGGGU | | | | | AGAGCAGAG | |
| 1537400 | 1537408 | AGGGGUGCAGAU | 2507 | 1835 | 1857 | 1537404 | CAGGGAGACAUC | 2694 |
| | | GUCUCCCUGCA | | | | | UGCACCCCU | |
| 1537401 | 1537411 | GCACCAUGCCAGG | 2508 | 1815 | 1837 | 1537407 | GCACCACACCUG | 2695 |
| | | UGUGGUGCAG | | | | | GCAUGGUGC | |
| 1537402 | 1537409 | CACUCCUGGCUGC | 2509 | 1855 | 1877 | 1537406 | UGAGUUGGGCAG | 2696 |
| | | CCAACUCAGG | | | | | CCAGGAGUG | |
| 1537403 | 1537410 | UUAUUAUCCAUUC | 2510 | 1875 | 1897 | 1537405 | GCCCCCGGGAAU | 2697 |
| | | CCGGGGGCAC | | | | | GGAUAAUAA | |

### Example 13: Effect of RNAi Compounds on Human SPDEF RNA In Vitro, Single Dose

Double-stranded RNAi compounds described above were tested in a series of experiments under the same culture conditions. The results for each experiment are presented in separate tables below.

Cultured VCaP cells at a density of 25000 cells per well were transfected using Lipofectamine 2000 with 500 nM of double-stranded RNAi. After a treatment period of approximately 24 hours, RNA was isolated from the cells and SPDEF RNA levels were measured by quantitative real-time RTPCR. Human primer probe set RTS35007 (described herein above) was used to measure RNA levels. Data was confirmed using a second human primer probe set, RTS35006 (forward sequence CACCTGGACATCTGGAAGTC, designated herein as SEQ ID NO: 2321; reverse sequence CCTTGAGGAACTGCCACAG, designated herein as SEQ ID NO: 2322; probe sequence AGTGAGGAGAGCTGGACCGACA, designated herein as SEQ ID NO: 2323). SPDEF RNA levels were normalized to total RNA content, as measured by RIBOGREEN®. Results are presented as percent change of SPDEF RNA, relative to PBS control (% control). The symbol “f” indicates that the modified oligonucleotide is complementary to the target transcript within the amplicon region of the primer probe set and so the associated data is not reliable. In such instances, additional assays using alternative primer probes must be performed to accurately assess the potency and efficacy of such modified oligonucleotides.

TABLE 102
Reduction of SPDEF RNA by RNAi

| Compound | SPDEF (% control) | SPDEF (% control) |
|---|---|---|
| ID | @500 nM RTS35006 | @ 500 nM RTS35007 |
| 1527452 | 100 | 109 |
| 1527453 | 93 | 127 |
| 1527454 | 122 | 118 |
| 1527455 | 96 | 119 |
| 1527456 | 99 | 112 |
| 1527457 | 99 | 119 |
| 1527471 | 74 | 85 |
| 1527474 | 111 | 125 |
| 1527475 | 94 | 109 |
| 1527488 | 96 | 122 |
| 1527489 | 93 | 103 |
| 1527490 | 54 | 54 |
| 1527491 | 113 | 122 |
| 1527492 | 90 | 113 |
| 1527493 | 76 | 98 |
| 1527506 | 121 | 117 |
| 1527508 | 63 | 83 |
| 1527509 | 91 | 109 |
| 1527511 | 88 | 108 |
| 1527524 | 87 | 88 |
| 1527525 | 84 | 96 |
| 1527526 | 78 | 97 |
| 1527527 | 98 | 123 |
| 1527528 | 82 | 110 |
| 1527529 | 56 | 65 |
| 1527542 | 32 | 41 |
| 1527545 | 91 | 105 |
| 1527546 | 79 | 100 |
| 1527547 | 83 | 97 |
| 1527560 | 63 | 49 |
| 1527561 | 89 | 93 |
| 1527562 | 87 | 111 |
| 1527563 | 72 | 97 |
| 1527564 | 83 | 105 |
| 1527565 | 81 | 97 |
| 1527578 | 73 | 95 |
| 1527579 | 62 | 71 |
| 1527582 | 66 | 77 |
| 1527583 | 78 | 85 |
| 1527596 | 113 | 109 |
| 1527599 | 78 | 85 |
| 1527600 | 95 | 96 |
| 1527601 | 20‡ | 43 |
| 1527602 | 85‡ | 97 |
| 1527603 | 60‡ | 78 |
| 1527614 | 25‡ | 71 |
| 1527616 | 25‡ | 57 |
| 1527617 | 65‡ | 117 |
| 1527618 | 41‡ | 71 |
| 1527619 | 89 | 135 |
| 1527632 | 48 | 35‡ |
| 1527633 | 66 | 65‡ |
| 1527634 | 118 | 97‡ |
| 1527635 | 34 | 43‡ |
| 1527637 | 86 | 98‡ |
| 1527650 | 80 | 108‡ |
| 1527652 | 72 | 69 |
| 1527653 | 65 | 87 |
| 1527654 | 55 | 66 |
| 1527655 | 31 | 22‡ |
| 1527668 | 72 | 80 |
| 1527669 | 76 | 112 |
| 1527670 | 83 | 93 |
| 1527672 | 106 | 126 |
| 1527686 | 86 | 111 |
| 1527687 | 83 | 116 |
| 1527688 | 75 | 102 |
| 1527689 | 78 | 113 |
| 1527690 | 76 | 108 |
| 1527691 | 77 | 105 |
| 1527705 | 72 | 97 |
| 1527706 | 60 | 82 |
| 1527707 | 68 | 94 |
| 1527708 | 71 | 97 |
| 1527722 | 81 | 109 |
| 1527723 | 31 | 43 |
| 1527724 | 60 | 86 |
| 1527725 | 76 | 103 |
| 1527726 | 73 | 102 |

TABLE 103
Reduction of SPDEF RNA by RNAi

| Compound | SPDEF (% control) | SPDEF (% control) |
|---|---|---|
| ID | @500 nM RTS35006 | @ 500 nM RTS35007 |
| 1528323 | 30‡ | 94 |
| 1528361 | 33 | 67‡ |
| 1528397 | 29 | 87 |
| 1537130 | 88 | 143 |
| 1537131 | 114 | 110 |
| 1537132 | 97 | 122 |
| 1537133 | 84 | 100 |
| 1537134 | 81 | 102 |
| 1537140 | 83 | 108 |
| 1537148 | 90 | 75 |
| 1537149 | 91 | 112 |
| 1537151 | 71 | 98 |
| 1537152 | 88 | 105 |
| 1537166 | 88 | 103 |
| 1537167 | 68 | 82 |
| 1537168 | 90 | 93 |
| 1537169 | 110 | 106 |
| 1537170 | 43 | 37 |
| 1537171 | 126 | 120 |
| 1537184 | 86 | 98 |
| 1537185 | 75 | 101 |
| 1537186 | 77 | 111 |
| 1537187 | 74 | 89 |
| 1537202 | 78 | 97 |
| 1537204 | 47 | 41 |
| 1537205 | 67 | 87 |
| 1537206 | 34 | 49 |
| 1537207 | 102 | 95 |
| 1537220 | 86 | 87 |
| 1537221 | 88 | 100 |
| 1537223 | 69 | 84 |
| 1537225 | 49 | 59 |
| 1537238 | 103 | 112 |
| 1537239 | 99 | 103 |
| 1537240 | 60 | 78 |
| 1537241 | 43 | 45 |
| 1537242 | 90 | 104 |
| 1537243 | 70 | 61 |
| 1537256 | 34 | 42 |
| 1537258 | 76 | 74 |
| 1537259 | 90 | 95 |
| 1537260 | 96 | 115 |
| 1537274 | 112 | 96 |
| 1537275 | 84‡ | 92 |
| 1537276 | 124 | 97 |
| 1537277 | 100 | 77 |
| 1537287 | 58‡ | 88 |
| 1537292 | 17‡ | 47 |
| 1537293 | 15‡ | 88 |
| 1537294 | 28‡ | 29 |
| 1537295 | 13‡ | 93 |
| 1537296 | 39‡ | 100 |
| 1537311 | 82 | 65‡ |
| 1537312 | 41 | 1‡ |
| 1537313 | 32 | 30‡ |
| 1537315 | 29 | 7‡ |
| 1537329 | 70 | 99 |
| 1537330 | 70 | 97 |
| 1537331 | 72 | 90 |
| 1537332 | 28 | 2‡ |
| 1537333 | 72 | 84 |
| 1537347 | 69 | 94 |
| 1537348 | 85 | 89 |
| 1537349 | 103 | 115 |
| 1537351 | 76 | 90 |
| 1537364 | 59 | 81 |
| 1537365 | 54 | 58 |
| 1537366 | 68 | 100 |
| 1537368 | 70 | 98 |
| 1537382 | 72 | 92 |
| 1537383 | 97 | 108 |
| 1537384 | 58 | 71 |
| 1537385 | 53 | 77 |
| 1537386 | 65 | 82 |
| 1537387 | 80 | 90 |
| 1537400 | 80 | 110 |
| 1537401 | 83 | 99 |
| 1537402 | 71 | 91 |
| 1537403 | 29 | 30 |

TABLE 104
Reduction of SPDEF RNA by RNAi

| Compound | SPDEF (% control) | SPDEF (% control) |
|---|---|---|
| ID | @500 nM RTS35006 | @ 500 nM RTS35007 |
| 1527470 | 88 | 97 |
| 1527473 | 98 | 99 |
| 1527507 | 108 | 104 |
| 1527510 | 91 | 100 |
| 1527543 | 69 | 73 |
| 1527544 | 87 | 91 |
| 1527580 | 64 | 57 |
| 1527581 | 105 | 101 |
| 1527615 | 40‡ | 53 |
| 1527636 | 74‡ | 84 |
| 1527651 | 27 | 33 |
| 1527671 | 94 | 97 |
| 1527673 | 76 | 80 |
| 1527704 | 98 | 101 |
| 1527709 | 81 | 79 |
| 1528231 | 67 | 79 |
| 1537153 | 94 | 107 |
| 1537188 | 88 | 91 |
| 1537189 | 78 | 85 |
| 1537222 | 89 | 100 |
| 1537224 | 97 | 103 |
| 1537257 | 73 | 80 |
| 1537261 | 61 | 61 |
| 1537297 | 10‡ | 76 |
| 1537314 | 99 | 83‡ |
| 1537346 | 92 | 89 |
| 1537350 | 98 | 100 |
| 1537367 | 88 | 92 |
| 1537369 | 84 | 88 |

1. An oligomeric compound comprising a modified oligonucleotide consisting of 16 linked nucleosides having a nucleobase sequence of SEQ ID NO: 1129, wherein the modified oligonucleotide has a sugar motif comprising:
a 5′-region consisting of 3 linked 5′-region nucleosides;
a central region consisting of 10 linked central region nucleosides; and
a 3′-region consisting of 3 linked 3′-region nucleosides,
wherein each of the 5′-region nucleosides and each of the 3′-region nucleosides comprises a cEt sugar moiety; each of the central region nucleosides comprises an unmodified 2′-deoxyribosyl sugar moiety; and each internucleoside linkage is a phosphorothioate linkage.

2. The oligomeric compound of claim 1, comprising a conjugate group.

3. The oligomeric compound of claim 1, wherein the oligomeric compound is single-stranded.

4. A pharmaceutical composition comprising the oligomeric compound of claim 1, and a pharmaceutically acceptable carrier or diluent.

5. The pharmaceutical composition of claim 4, wherein the pharmaceutically acceptable diluent comprises phosphate buffered saline (PBS).

6. The pharmaceutical composition of claim 5, consisting essentially of the oligomeric compound and PBS.

7. A method comprising administering to a subject the oligomeric compound of claim 1.

8. A method of treating a pulmonary condition comprising administering to a subject having or at risk for developing the pulmonary condition a therapeutically effective amount of the oligomeric compound of claim 1, thereby treating the pulmonary condition.

9. The method of claim 8, wherein the pulmonary condition is selected from bronchitis, asthma, chronic obstructive pulmonary disease, pulmonary fibrosis, idiopathic pulmonary fibrosis (IPF), pneumonia, emphysema, rhinitis, sinusitis, nasal polyposis, sinus polyposis, bronchiectasis, and sarcoidosis.

10. The method of claim 8, wherein the pulmonary condition is bronchitis.

11. The method of claim 8, wherein the pulmonary condition is asthma.

12. The method of claim 8, wherein administering comprises inhaling the oligomeric compound.

13. A chirally enriched population of the oligomeric compound of claim 1, wherein the population is enriched for oligomeric compounds having a modified oligonucleotide comprising at least one particular phosphorothioate internucleoside linkage having a particular stereochemical configuration.

14. A modified oligonucleotide according to the following chemical structure: [chemical structure]
or a salt thereof.

15. The modified oligonucleotide of claim 14, which is the sodium salt or the potassium salt.

16. A pharmaceutical composition comprising the modified oligonucleotide of claim 14, and a pharmaceutically acceptable carrier or diluent.

17. The pharmaceutical composition of claim 16, wherein the pharmaceutically acceptable diluent comprises PBS.

18. The pharmaceutical composition of claim 17, consisting essentially of the modified oligonucleotide and PBS.

19. A method comprising administering to a subject the modified oligonucleotide of claim 14.

20. A method of treating a pulmonary condition comprising administering to a subject having or at risk for developing the pulmonary condition a therapeutically effective amount of the modified oligonucleotide of claim 14, thereby treating the pulmonary condition.

21. The method of claim 20, wherein the pulmonary condition is selected from bronchitis, asthma, chronic obstructive pulmonary disease, pulmonary fibrosis, idiopathic pulmonary fibrosis (IPF), pneumonia, emphysema, rhinitis, sinusitis, nasal polyposis, sinus polyposis, bronchiectasis, and sarcoidosis.

22. The method of claim 20, wherein the pulmonary condition is bronchitis.

23. The method of claim 20, wherein the pulmonary condition is asthma.

24. The method of claim 20, wherein administering comprises inhaling the oligomeric compound.

25. A modified oligonucleotide according to the following chemical structure:

[chemical structure]

26. A pharmaceutical composition comprising the modified oligonucleotide of claim 25, and a pharmaceutically acceptable carrier or diluent.

27. The pharmaceutical composition of claim 26, wherein the pharmaceutically acceptable diluent comprises PBS.

28. The pharmaceutical composition of claim 27, consisting essentially of the modified oligonucleotide and PBS.

29. A method comprising administering to a subject the modified oligonucleotide of claim 25.

30. The method of claim 29, wherein administering comprises inhaling the oligomeric compound.
